(19) United States
(12) Patent Application Publication
(10) Pub. No.: US 2026/0158024 A1
(43) Pub. Date: Jun. 11, 2026

(54) KRAS INHIBITORS

(71) Applicant: Incyte Corporation, Wilmington, DE (US)

(72) Inventors: Alexander Sokolsky, Wilmington, DE (US); Rocco Policarpo, III, Wilmington, DE (US); Xiaozhao Wang, Wilmington, DE (US); Wenhao Zhang, Wilmington, DE (US); Wenyu Zhu, Wilmington, DE (US); Suman Chakrabarty, Wilmington, DE (US); Chengtsung Lai, Wilmington, DE (US); Haolin Yin, Wilmington, DE (US); Nathaniel Greenwood, Wilmington, DE (US)

(21) Appl. No.: 19/386,467

(22) Filed: Nov. 12, 2025

Related U.S. Application Data
(60) Provisional application No. 63/719,839, filed on Nov. 13, 2024.
(60) Provisional application No. 63/757,695, filed on Feb. 12, 2025.
(60) Provisional application No. 63/830,831, filed on Jun. 26, 2025.

(51) Int. Cl. A61K 31/4745; A61K 31/497; A61K 31/4995; A61K 31/501; A61P 35/00; C07B 59/00; C07D 471/06; C07D 519/00

(52) U.S. Cl. CPC A61K 31/4745; A61K 31/497; A61K 31/4995; A61K 31/501; A61P 35/00; C07B 59/002; C07D 471/06; C07D 519/00

(57) ABSTRACT

Disclosed are compounds of Formula (I), methods of using the compounds for inhibiting KRAS activity and pharmaceutical compositions comprising such compounds. The compounds are useful in treating, preventing or ameliorating diseases or disorders associated with KRAS activity such as cancer.

### RELATED APPLICATIONS

This application claims priority to U.S. Provisional Application No. 63/719,839, filed Nov. 13, 2024, U.S. Provisional Application No. 63/757,695, filed Feb. 12, 2025, and U.S. Provisional Application No. 63/830,831, filed Jun. 26, 2025, the content of each of which is incorporated in its entirety.

### FIELD OF THE INVENTION

This disclosure provides compounds as well as their compositions and methods of use. The compounds modulate KRAS activity and are useful in the treatment of various diseases including cancer.

### BACKGROUND OF THE INVENTION

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al., Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al., Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

### SUMMARY

The present disclosure provides, inter alia, a compound of Formula (I):

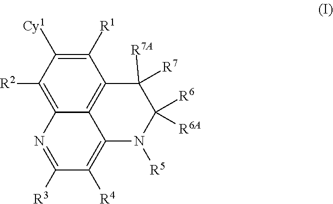

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure further provides methods of inhibiting KRAS activity, which comprises administering to an individual a compound of the disclosure, or a pharmaceutically acceptable salt thereof. The present disclosure also provides uses of the compounds described herein in the manufacture of a medicament for use in therapy. The present disclosure also provides the compounds described herein for use in therapy.

The present disclosure further provides methods of treating a disease or disorder in a patient comprising administering to the patient a therapeutically effective amount of a compound of the disclosure, or a pharmaceutically acceptable salt thereof.

The details of one or more embodiments are set forth in the description below. Other features, objects, and advantages will be apparent from the description and from the claims.

### DETAILED DESCRIPTION

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

### I. Compounds

In an aspect, provided herein is a compound having Formula (I):

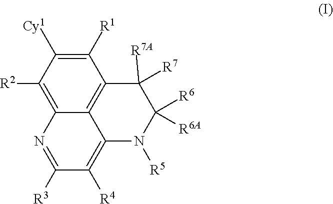

or a pharmaceutically acceptable salt thereof, wherein:
R¹ is selected from C₁₋₃ alkyl, C₁₋₃ haloalkyl, C₃₋₆ cycloalkyl, and halo;
Cy¹ is C₆₋₁₀ aryl or 6-10 membered heteroaryl, each optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{dCy1} is independently selected from H, C₁₋₃ alkyl, and C₁₋₃ haloalkyl;
R² is selected from C₁₋₃ alkyl and halo;
R³ is selected from H, C₁₋₃ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, halo, D, CN, OR^{a3}, NR^{b3}C(O)R^{a3}, C(O)NR^{b3}R^{c3}, N═S(O)R^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl, C₂₋₆ alkenyl, and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B}; or
R^{b3} and R^{c3} together with the nitrogen atom to which they are attached form a 4-10 membered heterocycloalkyl, optionally substituted with 1, 2, 3, or 4 substituents independently selected from R^{3A};
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, CN, OR^{d3}, C₃₋₁₀ cycloalkyl, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy, and 4-10 membered heterocycloalkyl forming R^{3B} is optionally substituted by 1, 2, 3 or 4 substituents independently selected from R^{3C};
each R^{3C} is independently selected from C₁₋₆ alkyl, C₂₋₆ alkenyl, C₂₋₆ alkynyl, halo, and C₁₋₆ haloalkyl, wherein C₁₋₆ alkyl, C₂₋₆ alkenyl, and C₂₋₆ alkynyl are each optionally substituted by 1, 2, or 3 halo;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy^{5A} is optionally substituted by Cy^{5A} or (C₃₋₆ alkylene)Cy^{5A} and also optionally substituted by 1, 2, 3, or 4 substituents each independently selected from R^{5C};
each Cy^{5A} is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy^{5A} is optionally substituted by 1, 2, 3, or 4 substituents each independently selected from R^{5C};
R^{5A} is selected from H, D, C₁₋₃ alkyl, C₁₋₃ haloalkyl, and C₃₋₆ cycloalkyl;
R^{5B} is selected from H, D, C₁₋₃ alkyl, and C₁₋₃ haloalkyl;
each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, OR^{a5}, SR^{a5}, S(O)R^{a5}, S(O)₂R^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a5}, R^{b5}, R^{c5}, and R^{d5} is independently selected from H and C₁₋₆ alkyl, wherein each C₁₋₆ alkyl forming each R^{a5}, R^{b5}, R^{c5}, and R^{d5} is optionally substituted by 1, 2, 3, or 4 substituents each independently selected from D and halo;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{e5} is independently selected from H, C₁₋₆ alkyl, OH and C₁₋₆ alkoxy, wherein each C₁₋₆ alkyl and C₁₋₆ alkoxy forming R^{e5} is optionally substituted by 1, 2, 3, or 4 substituents each independently selected from D and halo;
R⁶ is selected from H, D, C₁₋₃ alkyl and C₁₋₃ haloalkyl;
R^{6A} is selected from H, D, C₁₋₃ alkyl and C₁₋₃ haloalkyl;
R⁷ is selected from H, D, C₁₋₃ alkyl, C₁₋₃ haloalkyl, and halo; and
R^{7A} is selected from H, D, C₁₋₃ alkyl, C₁₋₃ haloalkyl, and halo; or
R⁷ and R^{7A} together with the carbon atom to which they are attached form a spiro fused C₃₋₇ cycloalkane ring;
wherein the ring-forming atoms of each heteroaryl and heterocycloalkyl consist of at least one ring-forming carbon atom and 1, 2, 3, or 4 heteroatoms independently selected from N, O, and S; wherein a ring-forming carbon atom of each heteroaryl and heterocycloalkyl is optionally substituted by oxo to form a carbonyl group.

In an embodiment,
R³ is selected from H, C₁₋₃ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, halo, D, CN, OR^{a3}, NR^{b3}C(O)R^{a3}, C(O)NR^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl, C₂₋₆ alkenyl, and C₂₋₆ alkynyl forming R³ is optionally substituted by 1, 2, 3, or 4 substituents each independently selected from R^{3B}; and
each R^{3B} is independently selected from halo, D, CN, OH, C₁₋₃ alkoxy, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy, and 4-10 membered heterocycloalkyl forming R^{3B} is optionally substituted by 1, 2, 3 or 4 substituents independently selected from R^{3C}.

In an embodiment,
R³ is selected from C₁₋₃ alkyl, C₃₋₁₀ cycloalkyl, 4-10 membered heterocycloalkyl, C₆₋₁₀ aryl, 5-10 membered heteroaryl, halo, OR^{a3}, C(O)NR^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl forming R³ is optionally substituted by 1, 2, 3, or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B}; or
R^{b3} and R^{c3} together with the nitrogen atom to which they are attached form a 4-10 membered heterocycloalkyl, optionally substituted with 1, 2, 3, or 4 substituents independently selected from R^{3A};
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, CN, OH, C₁₋₃ alkoxy, 4-10 membered heterocycloalkyl, amino, C₁₋₃ alkylamino and di(C₁₋₃ alkyl)amino, wherein the 4-10 membered heterocycloalkyl forming R^{3B} is optionally substituted by 1, 2, 3 or 4 substituents independently selected from R^{3C}; and
each R^{3C} is independently selected from C₁₋₆ alkyl, halo and C₁₋₆ haloalkyl.

In an embodiment,
R¹ is selected from C₁₋₃ alkyl and halo;
R³ is selected from H, C₁₋₃ alkyl, C₂₋₆ alkynyl, C₃₋₁₀ cycloalkyl, 4-10 membered heterocycloalkyl, C₆₋₁₀ aryl, 5-10 membered heteroaryl, D, CN, OR^{a3}, NR^{b3}C(O)R^{a3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3 or 4 substituents each independently selected from R^{3B};
each R^{3B} is independently selected from halo, D, CN, C₁₋₃ alkoxy, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy and 4-10 membered heterocycloalkyl forming R^{3B} is optionally substituted by 1, 2, 3 or 4 substituents independently selected from R^{3C};
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy⁵ is optionally substituted by 1, 2, 3, or 4 substituents each independently selected from R^{5C};
R^{5A} is selected from H, D, and C₁₋₃ alkyl;
R^{5B} is selected from H, D, and C₁₋₃ alkyl;
each R^{5C} is independently selected from C₁₋₃ alkyl and R^{5D}, wherein each C₁₋₃ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, C₁₋₃ alkoxy, amino, C₁₋₃ alkylamino, and di(C₁₋₃ alkyl)amino;
R⁷ is selected from H, D, C₁₋₃ alkyl and C₁₋₃ haloalkyl; and
R^{7A} is selected from H, D, C₁₋₃ alkyl, and C₁₋₃ haloalkyl; or
R⁷ and R^{7A} together with the carbon atom to which they are attached form a spiro fused C₃₋₇ cycloalkane ring.

In an embodiment,
R³ is selected from C₁₋₃ alkyl, C₃₋₁₀ cycloalkyl, 4-10 membered heterocycloalkyl, C₆₋₁₀ aryl, 5-10 membered heteroaryl, OR^{a3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl forming R³ is optionally substituted by 1, 2, 3 or 4 substituents each independently selected from R^{3B}; and
each R^{3B} is independently selected from halo, D, CN, C₁₋₃ alkoxy, 4-10 membered heterocycloalkyl, amino, C₁₋₃ alkylamino and di(C₁₋₃ alkyl)amino, wherein the 4-10 membered heterocycloalkyl forming R^{3B} is optionally substituted by 1, 2, 3 or 4 substituents independently selected from R^{3C}.

In some preferred embodiments:
R¹ is selected from C₁₋₃ alkyl and halo;
Cy¹ is C₆₋₁₀ aryl or 6-10 membered heteroaryl, each optionally substituted with 1, 2, or 3 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{dCy1} is independently selected from H, C₁₋₃ alkyl, and C₁₋₃ haloalkyl;
R² is selected from C₁₋₃ alkyl and halo;
R³ is selected from C₁₋₆ alkyl, C₃₋₆ cycloalkyl, 4-6 membered heterocycloalkyl, C₆₋₁₀ aryl, 5-10 membered heteroaryl, halo, D, and CN;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy⁵ is optionally substituted by 1, 2, 3, or 4 substituents each independently selected from R^{5C};
R^{5A} is selected from H, D, and C₁₋₃ alkyl;
R^{5B} is selected from H, D, and C₁₋₃ alkyl;
each R^{5C} is independently selected from C₁₋₃ alkyl and R^{5D}, wherein each C₁₋₃ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, C₁₋₃ alkoxy, amino, C₁₋₃ alkylamino, and di(C₁₋₃ alkyl)amino;
R⁶ is selected from H, D, and C₁₋₃ alkyl;
R^{6A} is selected from H, D, and C₁₋₃ alkyl;
R⁷ is selected from H, D, and C₁₋₃ alkyl; and
R^{7A} is selected from H, D, and C₁₋₃ alkyl; or
R⁷ and R^{7A} together with the carbon atom to which they are attached form a spiro fused C₃₋₇ cycloalkane ring.

In some preferred embodiments:
R¹ is selected from C₁₋₃ alkyl, C₁₋₃ haloalkyl, C₃₋₆ cycloalkyl, and halo;
Cy¹ is C₆₋₁₀ aryl or 6-10 membered heteroaryl, each optionally substituted with 1, 2, 3, or 4 substituents independently selected from R^{Cy1};
each R^{Cy1} is independently selected from C₁₋₃ alkyl, C₁₋₃ haloalkyl, C₂₋₃ alkynyl, halo, CN, and NR^{cCy1}R^{dCy1};
each R^{cCy1} and R^{dCy1} is independently selected from H and C₁₋₃ alkyl;
R² is selected from C₁₋₃ alkyl and halo;
R³ is selected from H, C₁₋₃ alkyl, C₂₋₆ alkynyl, C₃₋₁₀ cycloalkyl, 4-10 membered heterocycloalkyl, C₆₋₁₀ aryl, 5-10 membered heteroaryl, halo, D, CN, OR^{a3}, NR^{b3}C(O)R^{a3}, C(O)NR^{b3}R^{c3}, N═S(O)R^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B};
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, OH, OR^{d3}, C₃₋₁₀ cycloalkyl, S(O)(═NH)C₁₋₆ alkyl, 4-10 membered heterocycloalkyl, amino, C₁₋₃ alkylamino and di(C₁₋₃ alkyl)amino, wherein the 4-10 membered heterocycloalkyl forming R^{3B} is optionally substituted by 1, 2, 3 or 4 substituents independently selected from R^{3C};
each R^{3C} is independently selected from C₁₋₆ alkyl, C₂₋₆ alkenyl, halo and C₁₋₆ haloalkyl wherein C₂₋₆ alkenyl is optionally substituted by 1, 2, or 3 halo;
R⁴ is selected from H, D, and halo;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy⁵ is optionally substituted by Cy^{5A} and also optionally substituted by 1, 2, 3, or 4 substituents each independently selected from R^{5C};
each Cy^{5A} is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy^{5A} is optionally substituted by 1, 2, 3, or 4 substituents each independently selected from R^{5C};
R^{5A} is selected from H, D, C₁₋₃ alkyl, and C₃₋₆ cycloalkyl;
R^{5B} is selected from H, D, C₁₋₃ alkyl, and C₁₋₃ haloalkyl;
each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, OR^{a5}, C(O)R^{b5}, C(O)NR^{c5}R^{d5}, C(O)OR^{a5}, OC(O)R^{b5}, NR^{c5}R^{d5}, and NR^{c5}C(O)R^{b5};
each R^{a5}, R^{b5}, R^{c5}, and R^{d5} is independently selected from H and C₁₋₆ alkyl, wherein each C₁₋₆ alkyl forming each R^{a5}, R^{b5}, R^{c5}, and R^{d5} is optionally substituted by 1, 2, 3, or 4 substituents each independently selected from D and halo;
R⁶ is selected from H, D, and C₁₋₃ alkyl;
R^{6A} is H or D;
R⁷ is selected from H, D, C₁₋₃ alkyl, and halo; and
R^{7A} is selected from H, C₁₋₃ alkyl, C₁₋₃ haloalkyl, and halo.

In an embodiment,
R³ is selected from H, C₁₋₃ alkyl, C₂₋₆ alkynyl, C₃₋₁₀ cycloalkyl, 4-10 membered heterocycloalkyl, C₆₋₁₀ aryl, 5-10 membered heteroaryl, halo, D, CN, OR^{a3}, NR^{b3}C(O)R^{a3}, C(O)NR^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B};
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, OH, C₁₋₃ alkoxy, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy and 4-10 membered heterocycloalkyl forming R^{3B} is optionally substituted by 1, 2, 3 or 4 substituents independently selected from R^{3C}; and
each R^{3C} is independently selected from C₁₋₆ alkyl, C₂₋₆ alkenyl, halo and C₁₋₆ haloalkyl wherein C₂₋₆ alkenyl is optionally substituted by 1, 2, or 3 halo.

In some preferred embodiments:
R¹ is selected from C₁₋₃ alkyl and halo;
Cy¹ is C₆₋₁₀ aryl or 6-10 membered heteroaryl, each optionally substituted with 1, 2, or 3 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{dCy1} is independently selected from H, C₁₋₃ alkyl, and C₁₋₃ haloalkyl;
R² is selected from C₁₋₃ alkyl and halo;
R³ is selected from H, C₁₋₆ alkyl, C₂₋₆ alkynyl, C₃₋₆ cycloalkyl, 4-6 membered heterocycloalkyl, C₆₋₁₀ aryl, 5-10 membered heteroaryl, halo, D, CN, NR^{b3}C(O)R^{a3}, N═S(O)R^{b3}R^{c3}, and C(O)NR^{b3}R^{c3}; wherein the 4-10 membered heterocycloalkyl forming R³ is optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3} and R^{c3} is independently selected from H and C₁₋₆ alkyl;
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B};
each R^{3B} is independently di(C₁₋₃ alkyl)amino, S(O)(═NH)C₁₋₆ alkyl, OR^{d3}, or C₃₋₁₀ cycloalkyl;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C};
each Cy^{5A} is independently selected from C₃₋₁₀ cycloalkyl and C₆₋₁₀ aryl;
R^{5A} is selected from H, D, C₁₋₃ alkyl, and C₃₋₆ cycloalkyl;
R^{5B} is selected from H, D, and C₁₋₃ alkyl;
each R^{5C} is independently selected from C₁₋₃ alkyl and R^{5D}, wherein each C₁₋₃ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, C₁₋₃ alkoxy, C(O)R^{b5}, and NR^{c5}R^{d5};
each R^{b5}, R^{c5}, and R^{d5} is independently selected from H and C₁₋₆ alkyl;
R⁶ is selected from H, D, and C₁₋₃ alkyl;
R^{6A} is selected from H, D, and C₁₋₃ alkyl;
R⁷ is selected from H, D, halo, and C₁₋₃ alkyl; and
R^{7A} is selected from H, D, halo, and C₁₋₃ alkyl; or
R⁷ and R^{7A} together with the carbon atom to which they are attached form a spiro fused C₃₋₇ cycloalkane ring.

In an embodiment,
R³ is selected from H, C₁₋₆ alkyl, C₂₋₆ alkynyl, C₃₋₆ cycloalkyl, 4-6 membered heterocycloalkyl, C₆₋₁₀ aryl, 5-10 membered heteroaryl, halo, D, CN, NR^{b3}C(O)R^{a3}, and C(O)NR^{b3}R^{c3}; wherein the 4-10 membered heterocycloalkyl forming R³ is optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{3B} is independently di(C₁₋₃ alkyl)amino, S(O)(═NH)C₁₋₆ alkyl, or C₁₋₃ alkoxy optionally substituted by 1, 2, 3 or 4 substituents independently selected from R^{3C}; and
each R^{3C} is independently selected from C₁₋₆ alkyl, C₂₋₆ alkenyl, halo, and C₁₋₆ haloalkyl, wherein C₂₋₆ alkenyl is optionally substituted by 1, 2, or 3 halo.

In an embodiment,
R³ is selected from C₁₋₆ alkyl, C₃₋₆ cycloalkyl, 4-6 membered heterocycloalkyl, C₆₋₁₀ aryl, 5-10 membered heteroaryl, halo, D, CN, and C(O)NR^{b3}R^{c3}; wherein the 4-10 membered heterocycloalkyl forming R³ is optionally substituted with 1, 2, 3, or 4 substituents independently selected from R^{3A}; and
each R^{b3} and R^{c3} is independently selected from H and C₁₋₆ alkyl;
each R^{3A} is independently selected from C₁₋₆ alkyl and R^{3B};
each R^{3B} is independently di(C₁₋₃ alkyl)amino.

In some preferred embodiments:
R¹ is halo;
Cy¹ is C₆₋₁₀ aryl or 6-10 membered heteroaryl, each optionally substituted with 1, 2, or 3 substituents independently selected from R^{Cy1};
each R^{Cy1} is independently selected from C₁₋₃ alkyl, C₁₋₃ haloalkyl, C₂₋₃ alkynyl, halo, CN, and NH₂,
R² is halo;
R³ is C₁₋₆ alkyl;
R⁴ is selected from H, D, C₁₋₃ alkyl, and halo;
R⁵ is CR^{5A}R^{5B}Cy⁵;
each Cy⁵ is independently selected from C₆₋₁₀ aryl and 5-10 membered heteroaryl, wherein the C₆₋₁₀ aryl and 5-10 membered heteroaryl forming Cy⁵ is optionally substituted by 1, 2, or 3 substituents each independently selected from R^{5C};
R^{5A} is selected from H, D, and C₁₋₃ alkyl;
R^{5B} is selected from H, D, and C₁₋₃ alkyl;
each R^{5C} is independently selected from C₁₋₃ alkyl, halo, D, CN, OH, C₁₋₃ alkoxy, amino, C₁₋₃ alkylamino, and di(C₁₋₃ alkyl)amino;
R⁶ is H;
R^{6A} is H;
R⁷ is H; and
R^{7A} is H.

In some preferred embodiments,
R¹ is halo;
Cy¹ is 6-10 membered heteroaryl optionally substituted with 1, 2, 3, or 4 substituents independently selected from R^{Cy1};
each R^{Cy1} is independently selected from C₁₋₃ alkyl, halo, CN, and NR^{cCy1}R^{dCy1};
each R^{cCy1} and R^{dCy1} is H;
R² is halo;
R³ is selected from C₁₋₃ alkyl, 4-10 membered heterocycloalkyl, and C(O)NR^{b3}R^{c3}; wherein the 4-10 membered heterocycloalkyl forming R³ is optionally substituted with 1, 2, 3, or 4 substituents independently selected from R^{3A};
each R^{b3} and R^{c3} is independently selected from H and C₁₋₆ alkyl;
each R^{3A} is independently selected from C₁₋₆ alkyl and R^{3B};
each R^{3B} is independently di(C₁₋₃ alkyl)amino;
R⁴ is H;
R⁵ is selected from Cy⁵ and CR^{5A}R^{5B}Cy⁵;
each Cy⁵ is independently selected from 4-10 membered heterocycloalkyl and 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C};
each Cy^{5A} is independently selected from C₃₋₁₀ cycloalkyl and 06-10 aryl;
R^{5A} is selected from H, D, C₁₋₃ alkyl, and C₃₋₆ cycloalkyl;
R^{5B} is selected from H, D, and C₁₋₃ alkyl;
each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D};
each R^{5D} is independently selected from halo, D, C(O)R^{b5}, and NR^{c5}R^{d5};
each R^{b5}, R^{c5}, and R^{d5} is independently selected from H and C₁₋₆ alkyl;
R⁶ is H;
R^{6A} is H;
R⁷ is selected from H and halo; and
R^{7A} is selected from H and halo.

In some preferred embodiments:
R¹ is halo;
Cy¹ is 6-10 membered heteroaryl optionally substituted with 1, 2, 3, or 4 substituents independently selected from R^{Cy1};
each R^{Cy1} is independently selected from halo, CN, and NR^{cCy1}R^{dCy1};
R^{cCy1} and R^{dCy1} is H;
R² is halo;
R³ is selected from C₁₋₃ alkyl, 4-10 membered heterocycloalkyl, and C(O)NR^{b3}R^{c3}; wherein the 4-10 membered heterocycloalkyl forming R³ is optionally substituted with 1, 2, 3, or 4 substituents independently selected from R^{3A};
each R^{a3}, R^{b3}, and R^{c3} is independently selected from H and C₁₋₆ alkyl;
each R^{3A} is independently selected from C₁₋₆ alkyl and R^{3B};
each R^{3B} is independently di(C₁₋₃ alkyl)amino;
R⁴ is H;
R⁵ is selected from Cy⁵ and CR^{5A}R^{5B}Cy⁵;
each Cy⁵ is independently selected from 4-10 membered heterocycloalkyl and 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C};
each Cy^{5A} is independently selected from C₃₋₁₀ cycloalkyl and C₆₋₁₀ aryl;
R^{5A} is selected from H, D, C₁₋₃ alkyl, and C₃₋₆ cycloalkyl;
R^{5B} is selected from H, D, and C₁₋₃ alkyl;
each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D};
each R^{5D} is independently selected from halo, D, C(O)R^{b5}, and NR^{c5}R^{d5};
each R^{a5}, R^{b5}, R^{c5}, and R^{d5} is independently selected from H and C₁₋₆ alkyl;
R⁶ is H;
R^{6A} is H;
R⁷ is selected from H and halo; and
R^{7A} is selected from H and halo.

In some preferred embodiments, the compound of Formula I is a compound of any one of Formulae I-A to I-H:

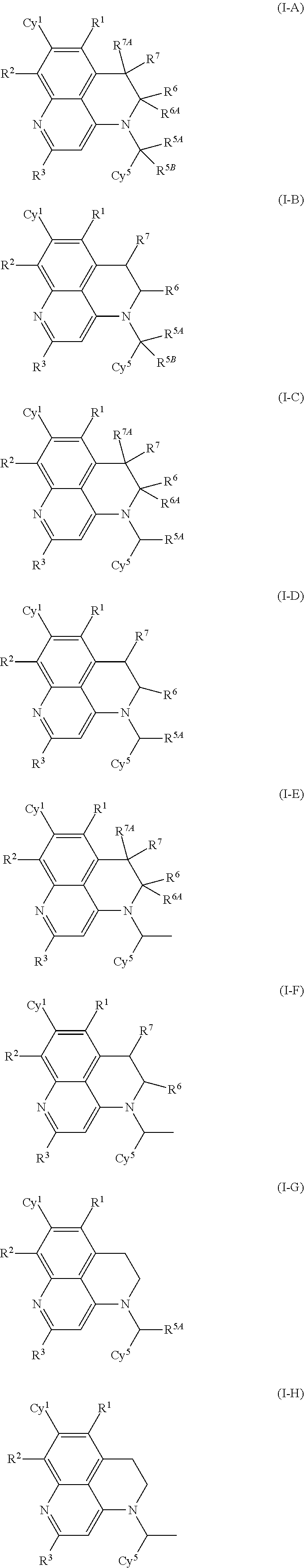

or a pharmaceutically acceptable salt thereof.

In some preferred embodiment, the compound of Formula I is a compound of Formula I-G, wherein:
R³ is C₁₋₃ alkyl optionally substituted by 1 or 2 substituents each independently selected from R^{3B}
Cy⁵ is pyridinyl optionally substituted with amino; and
R^{5A} is C₁₋₃ alkyl.

In some embodiments: the compound of Formula I is a compound of Formula (I-I) or (I-J):

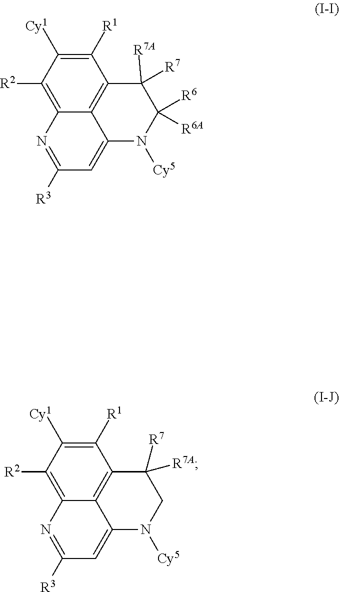

or a pharmaceutically acceptable salt thereof.

In some embodiments, the compound of Formula I is a compound of Formula (I-J) or Formula (I-K):

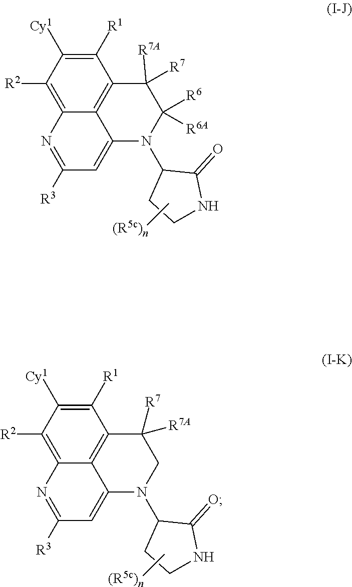

or a pharmaceutically acceptable salt thereof;
wherein n is 0, 1, or 2.

In some embodiments, R¹ is halo (e.g., F or Cl).

In some embodiments, R¹ is Cl.

In some embodiments, Cy¹ is phenyl, napthyl, or 6-10 membered heteroaryl, each optionally substituted with 1, 2, 3, or 4 substituents (or 1, 2, or 3 substituents, or 1 or 2 substituents, or 1 substituent) independently selected from R^{Cy1}.

In some embodiments, Cy¹ is C₆₋₁₀ aryl, benzothiophenyl, benzothiazolyl, or indazolyl,
each optionally substituted with 1, 2, 3 or 4 substituents (or 1, 2, or 3 substituents, or 1 or 2 substituents, or 1 substituent) independently selected from R^{Cy1}.

In some embodiments, Cy¹ is C₆₋₁₀ aryl or 6-10 membered heteroaryl, each optionally substituted with 1, 2, 3, or 4 substituents (or 1, 2, or 3 substituents, or 1 or 2 substituents, or 1 substituent) independently selected from C₁₋₃ alkyl, C₂₋₆ alkynyl, halo, OH, and NH₂.

In some embodiments, Cy¹ is selected from phenyl, naphthyl (such as 1-naphthyl and 2-naphthyl), benzothiazolyl (such as benzothiazol-2-yl, -4-yl, -5-yl, -6-yl, or -7-yl), benzothiophenyl (such as benzothiophen-2-yl, -4-yl, -5-yl, -6-yl, or -7-yl), and indazolyl (such as indazo-2-yl, -3-yl, -4-yl, -5-yl, or -6-yl) each optionally substituted with 1, 2, or 3 substituents independently selected from R^{Cy1}.

In some embodiments, each R^{Cy1} is independently selected from C₁₋₃ alkyl, C₂₋₃ alkynyl, C₁₋₃ haloalkyl, halo, CN, and NR^{cCy1}R^{dCy1}.

In some embodiments, each R^{Cy1} is independently selected from C₁₋₃ alkyl, halo, CN, and NR^{cCy1}R^{dCy1}.

In some embodiments, each R^{Cy1} is independently selected from C₁₋₃ alkyl, C₂₋₃ alkynyl, halo, CN, and NH₂.

In some embodiments, Cy¹ is selected from 2-amino-7-fluorobenzo[d]thiazol-4-yl, 2-amino-3-cyano-7-fluorobenzo[d]thiophen-4-yl, 1-cyanonaphth-8-yl, and 5,6-dimethyl-1H-indazol-4-yl

In some embodiments, R² is halo (e.g., F or Cl).

In some embodiments, R² is F.

In some embodiments,
R³ is selected from H, C₁₋₃ alkyl, C₂₋₆ alkynyl, 4-10 membered heterocycloalkyl, halo, CN, OR^{a3}, NR^{b3}C(O)R^{a3}, and C(O)NR^{b3}R^{c3}; wherein the 4-10 membered heterocycloalkyl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, 4-10 membered heterocycloalkyl, and 5-10 membered heteroaryl; wherein the 4-10 membered heterocycloalkyl and 5-10 membered heteroaryl forming R^{a3}, R^{b3}, and R^{c3} are each optionally substituted with 1, 2, or 3 substituents independently selected from R^{3A} and wherein the 1-6 alkyl forming R^{a3}, R^{b3} and R^{c3} is optionally substituted with 1, 2, or 3 substituents independently selected from R^{3B};
each R^{3A} is independently selected from C₁₋₆ alkyl and R^{3B}, wherein the C₁₋₆ alkyl forming R^{3A} is optionally substituted by 1, 2, or 3, substituents each independently selected from R^{3B};
each R^{3B} is independently selected from halo, OH, C₁₋₃ alkoxy, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy and 4-10 membered heterocycloalkyl forming R^{3B} is optionally substituted by 1, 2, 3 or 4 substituents independently selected from R^{3C}; and
each R^{3C} is independently selected from C₁₋₆ alkyl, C₂₋₆ alkenyl, and halo, wherein C₂₋₆ alkenyl is optionally substituted by 1, 2, or 3 halo.

In some embodiments,
R³ is selected from C₁₋₆ alkyl, C₃₋₆ cycloalkyl, 4-10 membered heterocycloalkyl, C₆₋₁₀ aryl, 5-10 membered heteroaryl, halo, D, CN, N═S(O)R^{b3}R^{c3}; wherein the C₃₋₆ cycloalkyl, 4-10 membered heterocycloalkyl, C₆₋₁₀ aryl, and 5-10 membered heteroaryl forming R³ are each optionally substituted with 1, 2, or 3 substituents independently selected from R^{3A}; and wherein the C₁₋₆ alkyl forming R³ is optionally substituted by 1, 2, or 3 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, or 3 substituents independently selected from R^{3B};
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, or 3 substituents independently selected from D, halo, CN, S(O₂)C₁₋₆ alkyl;
each R^{3A} is independently selected from C₁₋₆ alkyl and R^{3B}, wherein the C₁₋₆ alkyl forming R^{3A} is optionally substituted by 1, 2, or 3 substituents each independently selected from R^{3B};
each R^{3B} is independently selected from halo, D, CN, OR^{d3}, C₃₋₁₀ cycloalkyl, 4-10 membered heterocycloalkyl, S(O)(═NH)C₁₋₆ alkyl, amino, C₁₋₃ alkylamino and di(C₁₋₃ alkyl)amino, wherein 4-10 membered heterocycloalkyl forming R^{3B} is optionally substituted by 1, 2, or 3 substituents independently selected from R^{3C}; and
each R^{3C} is independently selected from C₁₋₆ alkyl, C₂₋₆ alkenyl, C₂₋₆ alkynyl, halo and C₁₋₆ haloalkyl, wherein C₂₋₆ alkenyl, and C₂₋₆ alkynyl are each optionally substituted by 1, 2, or 3 halo.

In some embodiments, R³ is selected from C₁₋₃ alkyl, 4-10 membered heterocycloalkyl, and C(O)NR^{b3}R^{c3}; wherein the 4-10 membered heterocycloalkyl forming R³ is optionally substituted with 1, 2, 3, or 4 substituents independently selected from R^{3A};

In some embodiments, R³ is 4-10 membered heterocycloalkyl forming R³ is optionally substituted with 1, 2, 3, or 4 substituents independently selected from R^{3A}. In some embodiments, R³ is 4-10 membered heterocycloalkyl forming R³ is optionally substituted with 1, 2, 3, or 4 substituents independently selected from R^{3A};

In some embodiments, R³ is H or C₁₋₃ alkyl. In some embodiments, R³ is selected from H, C₂₋₆ alkynyl, D, CN, and NR^{b3}C(O)R^{a3}, wherein the C₂₋₆ alkynyl forming R³ is optionally substituted by 1, 2, 3, or 4 (e.g., 1, 2, or 3, 1 or 2, or 1) substituents each independently selected from R^{3B}.

In some embodiments, each R^{b3} and R^{c3} is independently selected from H and C₁₋₆ alkyl.

In some embodiments, R³ is C₁₋₃ alkyl or halo.

In some embodiments, R³ is C₁₋₃ alkyl (e.g., methyl or ethyl).

In some embodiments, R³ is methyl.

In some embodiments, R³ is OR^{a3}.

In some embodiments, R^{a3} is C₁₋₆ alkyl (e.g., methyl) optionally substituted by 1, 2, 3, or 4 substituents independently selected from R^{3B}.

In some embodiments, R^{a3} is C₁₋₆ alkyl (e.g., methyl) optionally substituted by 1 substituent selected from R^{3B}.

In some embodiments, R^{a3} is methyl optionally substituted by 1 substituent selected from R^{3B}.

In some embodiments, at least one R^{3B} is 4-10 membered heterocycloalkyl optionally substituted by 1, 2, 3 or 4 (e.g., 1, 2, or 3, 1 or 2, or 1) R^{3C}.

In some embodiments, at least one R^{3B} is 5-6 membered heterocycloalkyl, wherein the at least one 5-6 membered heterocycloalkyl forming R^{3B} is optionally substituted by 1, 2, 3 or 4 (e.g., 1, 2, or 3, 1 or 2, or 1) R^{3C}.

In some embodiments, each R^{3B} is independently di(C₁₋₃ alkyl)amino.

In some embodiments, at least one R^{3C} is C₁₋₆ alkyl (e.g., methyl). In some such embodiments, each R^{3C} is C₁₋₆ alkyl (e.g., methyl).

In some embodiments, at least one R^{3C} is halo (e.g., fluoro). In some such embodiments, each R^{3C} is halo (e.g., fluoro).

In some embodiments, at least one R^{3C} is C₁₋₆ haloalkyl (e.g., C₁₋₆ fluoroalkyl or mono-, di-, or tri-halomethyl, such as fluoromethyl, difluoromethyl or trifluormethyl). In some such embodiments, each R^{3C} is C₁₋₆ haloalkyl (e.g., C₁₋₆ fluoroalkyl or mono-, di-, or tri-halomethyl, such as fluoromethyl, difluoromethyl or trifluormethyl).

In some embodiments, R³ is 4-10 membered heterocycloalkyl optionally substituted with 1, 2, 3, or 4 (e.g., 1, 2, or 3, 1 or 2, or 1) substituents independently selected from R^{3A}.

In some embodiments, R³ is 8-10 membered heterocycloalkyl optionally substituted with 1, 2, 3, or 4 (e.g., 1, 2, or 3, 1 or 2, or 1) substituents independently selected from R^{3A}.

In some embodiments, R³ is 8-10 membered fused bicyclic heterocycloalkyl optionally substituted with 1, 2, 3, or 4 (e.g., 1, 2, or 3, 1 or 2, or 1) substituents independently selected from R^{3A}.

In some embodiments, each R^{3A} is C₁₋₆ alkyl (e.g., methyl).

In some embodiments, each R^{3A} is independently selected from C₁₋₆ alkyl and R^{3B}.

In some embodiments, R⁴ is H or D.

In some embodiments, R⁴ is H.

In some embodiments, R⁵ is Cy⁵.

In some embodiments, R⁵ is CR^{5A}R^{5B}Cy⁵.

In some embodiments, R^{5A} is C₁₋₃ alkyl (e.g., methyl or ethyl).

In some embodiments, R^{5A} is C₁₋₃ haloalkyl (e.g., difluoromethyl or trifluoromethyl).

In some embodiments, R^{5B} is H.

In some embodiments, each Cy⁵ is independently selected from 4-10 membered heterocycloalkyl and 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C}.

In some embodiments, Cy⁵ is 5-10 membered heteroaryl optionally substituted by 1, 2, or 3 substituents (or 1 or 2 substituents, or 1 substituent) each independently selected from R^{5C}.

In some embodiments, Cy⁵ is pyridinyl (such as pyridin-2-yl, -3-yl, -4-yl, -5-yl, or -6-yl) optionally substituted with 1 or 2 substituents independently selected from R^{5C}.

In some embodiments, each Cy^{5A} is independently selected from C₃₋₁₀ cycloalkyl and C₆₋₁₀ aryl.

In some embodiments, R^{5A} is selected from H, D, C₁₋₃ alkyl, and C₃₋₆ cycloalkyl.

In some embodiments, R^{5B} is selected from H, D, and C₁₋₃ alkyl.

In some embodiments, each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D}.

In some embodiments, each R^{5C} is independently selected from amino, C₁₋₃ alkylamino, and di(C₁₋₃ alkyl)amino.

In some embodiments, each R^{5C} is amino.

In some embodiments, each R^{5D} is independently selected from halo, D, C(O)R^{b5}, and NR^{c5}R^{d5}.

In some embodiments, each R^{b5}, R^{c5}, and R^{d5} is independently selected from H and C₁₋₆ alkyl.

In some embodiments, Cy⁵ is 2-aminopyridin-3-yl.

In some embodiments, R⁶ is H.

In some embodiments, R^{6a} is H.

In some embodiments, R⁷ is H.

In some embodiments, R^{7a} is H.

In some embodiments, R⁷ is halo.

In some embodiments, R^{7a} is halo.

In other embodiments, the compound of Formula (I) is selected from the compounds in Table 1, and pharmaceutically acceptable salts thereof.

TABLE 1

| Entry | Compound Name |
|---|---|
| 1 | 2-amino-4-(4-(1-(2-aminopyridin-3-yl)ethyl)-7-chloro-9-fluoro-2-methyl-5,6-dihydro- |
| | 4H-benzo[de][1,6]naphthyridin-8-yl)-7-fluorobenzo[b]thiophene-3-carbonitrile |
| 2 | 4-(4-(1-(2-aminopyridin-3-yl)ethyl)-7-chloro-9-fluoro-2-methyl-5,6-dihydro-4H- |
| | benzo[de][1,6]naphthyridin-8-yl)-7-fluorobenzo[d]thiazol-2-amine |
| 3 | 8-(4-(1-(2-aminopyridin-3-yl)ethyl)-7-chloro-9-fluoro-2-methyl-5,6-dihydro-4H- |
| | benzo[de][1,6]naphthyridin-8-yl)-1-naphthonitrile |
| 4 | 3-(1-(7-chloro-8-(5,6-dimethyl-1H-indazol-4-yl)-9-fluoro-2-methyl-5,6-dihydro-4H- |
| | benzo[de][1,6]naphthyridin-4-yl)ethyl)pyridin-2-amine. |
| 5 | 2-amino-4-(7-chloro-9-fluoro-2-methyl-4-(1-(pyridin-3-yl)ethyl)-5,6-dihydro-4H- |
| | benzo[de][1,6]naphthyridin-8-yl)-7-fluorobenzo[b]thiophene-3-carbonitrile |
| 6 | 2-amino-4-(4-(1-(3-aminopyrazin-2-yl)ethyl)-7-chloro-9-fluoro-2-methyl-5,6-dihydro- |
| | 4H-benzo[de][1,6]naphthyridin-8-yl)-7-fluorobenzo[b]thiophene-3-carbonitrile |
| 7 | 2-amino-4-(4-(1-(3-aminopyridazin-4-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 8 | 2-amino-4-(4-(1-(5-aminopyridin-3-yl)ethyl)-7-chloro-9-fluoro-2-methyl-5,6-dihydro- |
| | 4H-benzo[de][1,6]naphthyridin-8-yl)-7-fluorobenzo[b]thiophene-3-carbonitrile |
| 9 | 2-amino-4-(7-chloro-9-fluoro-2-methyl-4-(1-(1-methyl-1H-pyrazol-5-yl)ethyl)-5,6- |
| | dihydro-4H-benzo[de][1,6]naphthyridin-8-yl)-7-fluorobenzo[b]thiophene-3- |
| | carbonitrile |
| 10 | 2-amino-4-(4-(1-(2-aminopyridin-3-yl)ethyl)-7-chloro-9-fluoro-2-methyl-5,6-dihydro- |
| | 4H-benzo[de][1,6]naphthyridin-8-yl)-7-fluorothieno[3,2-c]pyridine-3-carbonitrile |
| 11 | 2-amino-4-(4-(1-(2-aminopyridin-3-yl)ethyl)-7-chloro-9-fluoro-2,6-dimethyl-5,6- |
| | dihydro-4H-benzo[de][1,6]naphthyridin-8-yl)-7-fluorobenzo[b]thiophene-3- |
| | carbonitrile |
| 12 | 2-amino-4-(4-(1-(2-aminopyridin-3-yl)ethyl-1-d)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 13 | 2-amino-4-(4-(1-(2-amino-5-fluoropyridin-3-yl)ethyl-1-d)-7-chloro-9-fluoro-2-methyl- |
| | 5,6-dihydro-4H-benzo[de][1,6]naphthyridin-8-yl)-7-fluorobenzo[b]thiophene- |
| | 3-carbonitrile |
| 14 | 2-amino-4-(4-((2-aminopyridin-3-yl)(cyclopropyl)methyl-d)-7-chloro-9- |
| | fluoro-2-methyl-5,6-dihydro-4H-benzo[de][1,6]naphthyridin-8-yl)-7- |
| | fluorobenzo[b]thiophene-3-carbonitrile |
| 15 | 2-amino-4-(7-chloro-9-fluoro-2-methyl-4-(1-methyl-2-oxopyrrolidin-3-yl)-5,6- |
| | dihydro-4H-benzo[de][1,6]naphthyridin-8-yl)-7-fluorobenzo[b]thiophene-3- |
| | carbonitrile |
| 16 | 2-amino-4-(7-chloro-4-(1-cyclopropyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3- |
| | carbonitrile |
| 17 | 2-amino-4-(7-chloro-9-fluoro-2-methyl-4-(2-oxo-1-(2,2,2-trifluoroethyl) pyrrolidin-3- |
| | yl)-5,6-dihydro-4H-benzo[de][1,6]naphthyridin-8-yl)-7-fluorobenzo |
| | [b]thiophene-3-carbonitrile |
| 18 | 2-amino-4-(7-chloro-9-fluoro-2-methyl-4-(2-oxo-1-phenylpyrrolidin-3-yl)-5,6- |
| | dihydro-4H-benzo[de][1,6]naphthyridin-8-yl)-7-fluorobenzo[b]thiophene-3- |
| | carbonitrile |
| 19 | 2-amino-4-(7-chloro-9-fluoro-2-methyl-4-(1-methyl-2-oxopiperidin-4-yl)-5,6-dihydro- |
| | 4H-benzo[de][1,6]naphthyridin-8-yl)-7-fluorobenzo[b]thiophene-3-carbonitrile |
| 20 | 2-amino-4-(7-chloro-9-fluoro-2-methyl-4-(3-oxooctahydroindolizin-2-yl)-5,6-dihydro- |
| | 4H-benzo[de][1,6]naphthyridin-8-yl)-7-fluorobenzo[b]thiophene-3-carbonitrile |
| 21 | 2-amino-4-(7-chloro-9-fluoro-2-methyl-4-(1-methyl-2-oxopiperidin-3-yl)-5,6-dihydro- |
| | 4H-benzo[de][1,6]naphthyridin-8-yl)-7-fluorobenzo[b]thiophene-3-carbonitrile |
| 22 | 2-amino-4-(7-chloro-9-fluoro-2-methyl-4-(1-(1-methyl-1H-1,2,4-triazol-5-yl)ethyl)- |
| | 5,6-dihydro-4H-benzo[de][1,6]naphthyridin-8-yl)-7-fluorobenzo[b]thiophene- |
| | 3-carbonitrile |
| 23 | 2-amino-4-(7-chloro-9-fluoro-2-methyl-4-(1-(methyl-d₃)-2-oxopyrrolidin-3-yl)-5,6- |
| | dihydro-4H-benzo[de][1,6]naphthyridin-8-yl)-7-fluorobenzo[b]thiophene-3- |
| | carbonitrile |
| 24 | 4-(4-(1-acetylpyrrolidin-3-yl)-7-chloro-9-fluoro-2-methyl-5,6-dihydro-4H- |
| | benzo[de][1,6]naphthyridin-8-yl)-2-amino-7-fluorobenzo[b]thiophene-3-carbonitrile |
| 25 | 2-amino-4-(7-chloro-9-fluoro-2-methyl-4-(1-methyl-2- |
| | oxooctahydrocyclopenta[b]pyrrol-3-yl)-5,6-dihydro-4H-benzo[de][1,6] |
| | naphthyridin-8-yl)-7-fluorobenzo[b]thiophene-3-carbonitrile |
| 26 | 2-amino-4-(7-chloro-9-fluoro-2-methyl-4-(2-methyl-3-oxo-2-azabicyclo[3.1.0]hexan- |
| | 4-yl)-5,6-dihydro-4H-benzo[de][1,6]naphthyridin-8-yl)-7-fluorobenzo |
| | [b]thiophene-3-carbonitrile |
| 27 | 2-amino-4-(7-chloro-9-fluoro-2-methyl-4-(2-methyl-3-oxo-2- |
| | azabicyclo[3.2.0]heptan-4-yl)-5,6-dihydro-4H-benzo[de][1,6]naphthyridin-8- |
| | yl)-7-fluorobenzo[b]thiophene-3-carbonitrile |
| 28 | 2-amino-4-(7-chloro-2-(3-(dimethylamino)-3-methylazetidin-1-yl)-9-fluoro-4-(1- |
| | methyl-2-oxopyrrolidin-3-yl)-5,6-dihydro-4H-benzo[de][1,6]naphthyridin-8- |
| | yl)-7-fluorobenzo[b]thiophene-3-carbonitrile |
| 29 | 8-(2-amino-3-cyano-7-fluorobenzo[b]thiophen-4-yl)-4-(1-(2-aminopyridin-3- |
| | yl)ethyl)-7-chloro-9-fluoro-N-methyl-5,6-dihydro-4H-benzo[de][1,6] |
| | naphthyridine-2-carboxamide |
| 30 | 2-amino-4-(4-(1-(2-aminopyridin-3-yl)ethyl)-7-chloro-6,6,9-trifluoro-2-methyl-5,6- |
| | dihydro-4H-benzo[de][1,6]naphthyridin-8-yl)-7-fluorobenzo[b]thiophene-3- |
| | carbonitrile |
| 31 | 3-(8-(2-amino-7-fluorobenzo[d]thiazol-4-yl)-7-chloro-9-fluoro-2-methyl-5,6-dihydro- |
| | 4H-benzo[de][1,6]naphthyridin-4-yl)-1-methylpyrrolidin-2-one |
| 32 | 8-(2-amino-3-cyano-7-fluorobenzo[b]thiophen-4-yl)-7-chloro-9-fluoro-N-methyl-4- |
| | (1-methyl-2-oxopyrrolidin-3-yl)-5,6-dihydro-4H-benzo[de][1,6]naphthyridine-2- |
| | carboxamide |
| 33 | 2-amino-4-(7-chloro-9-fluoro-2-(1-hydroxyethyl)-4-(1-methyl-2-oxopyrrolidin-3-yl)- |
| | 5,6-dihydro-4H-benzo[de][1,6]naphthyridin-8-yl)-7-fluorobenzo[b]thiophene- |
| | 3-carbonitrile |
| 34 | 2-amino-4-(7-chloro-9-fluoro-2-methoxy-4-(1-methyl-2-oxopyrrolidin-3-yl)-5,6- |
| | dihydro-4H-benzo[de][1,6]naphthyridin-8-yl)-7-fluorobenzo [b]thiophene-3- |
| | carbonitrile |
| 35 | 8-(2-amino-3-cyano-7-fluorobenzo[b]thiophen-4-yl)-7-chloro-9-fluoro-4-(1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 36 | 8-(2-amino-3-cyano-7-fluorobenzo[b]thiophen-4-yl)-7-chloro-9-fluoro-N-(2-hydroxy- |
| | 2-methylpropyl)-4-(1-methyl-2-oxopyrrolidin-3-yl)-5,6-dihydro-4H- |
| | benzo[de][1,6]naphthyridine-2-carboxamide |
| 37 | N-(8-(2-amino-3-cyano-7-fluorobenzo[b]thiophen-4-yl)-7-chloro-9-fluoro-4-(1- |
| | methyl-2-oxopyrrolidin-3-yl)-5,6-dihydro-4H-benzo[de][1,6]naphthyridin-2- |
| | yl) acetamide |
| 38 | 2-amino-4-(7-chloro-2-((difluoromethoxy)methyl)-9-fluoro-4-(1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 39 | 2-amino-4-(7-chloro-9-fluoro-4-(4-hydroxy-1-(methyl-d₃)-2-oxopyrrolidin-3-yl)-2- |
| | methyl-5,6-dihydro-4H-benzo[de][1,6]naphthyridin-8-yl)-7-fluorobenzo[b]thiophene- |
| | 3-carbonitrile |
| 40 | 2-amino-4-(7-chloro-4-(4-(difluoromethoxy)-1-(methyl-d₃)-2-oxopyrrolidin-3-yl)-9- |
| | fluoro-2-methyl-5,6-dihydro-4H-benzo[de][1,6]naphthyridin-8-yl)-7- |
| | fluorobenzo[b]thiophene-3-carbonitrile |
| 41 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 42 | 2-amino-4-(7-chloro-4-(5-(difluoromethyl)-1-(methyl-d₃)-2-oxopyrrolidin-3-yl)-9- |
| | fluoro-2-methyl-5,6-dihydro-4H-benzo[de][1,6]naphthyridin-8-yl)-7- |
| | fluorobenzo[b]thiophene-3-carbonitrile |
| 43 | 2-amino-4-(7-chloro-9-fluoro-2-methyl-4-(4-(methyl-d₃)-5-oxo-4- |
| | azaspiro[2.4]heptan-6-yl)-5,6-dihydro-4H-benzo[de][1,6]naphthyridin-8-yl)- |
| | 7-fluorobenzo[b]thiophene-3-carbonitrile |
| 44 | 2-amino-4-(7-chloro-9-fluoro-4-(5-((methoxy-d3)methyl)-1-(methyl-d₃)-2- |
| | oxopyrrolidin-3-yl)-2-methyl-5,6-dihydro-4H-benzo[de][1,6]naphthyridin-8- |
| | yl)-7-fluorobenzo[b]thiophene-3-carbonitrile |
| 45 | 4-(8-(2-amino-3-cyano-7-fluorobenzo[b]thiophen-4-yl)-7-chloro-9-fluoro-2-methyl- |
| | 5,6-dihydro-4H-benzo[de][1,6]naphthyridin-4-yl)-N,N-dimethyl-1-(methyl- |
| | d₃)-5-oxopyrrolidine-2-carboxamide |
| 46 | 2-amino-4-(7-chloro-9-fluoro-2-methyl-4-(2-methyl-3-oxoisoxazolidin-4-yl)-5,6- |
| | dihydro-4H-benzo[de][1,6]naphthyridin-8-yl)-7-fluorobenzo[b]thiophene-3- |
| | carbonitrile |
| 47 | 2-amino-4-(7-chloro-9-fluoro-4-(1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 48 | 2-amino-4-(7-chloro-2-(3-(dimethylamino)azetidin-1-yl)-9-fluoro-4-(1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 49 | 2-amino-4-(7-chloro-2-(3-(dimethylamino)-3-methylazetidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 50 | 2-amino-4-(7-chloro-9-fluoro-4-(1-(methyl-d₃)-2-oxo-5-(trifluoromethyl)pyrrolidin-3- |
| | yl)-2-(3-(S-methylsulfonimidoyl) azetidin-1-yl)-5,6-dihydro-4H- |
| | benzo[de][1,6]naphthyridin-8-yl)-7-fluorobenzo[b]thiophene-3-carbonitrile |
| 51 | 2-amino-4-(7-chloro-9-fluoro-2-(3-methyl-3-(pyrrolidin-1-yl)azetidin-1-yl)-4-(1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 52 | 2-amino-4-(7-chloro-9-fluoro-2-((2-fluorotetrahydro-1H-pyrrolizin-7a(5H)- |
| | yl)methoxy)-4-(1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 53 | 2-amino-4-(7-chloro-9-fluoro-4-((1-(methyl-d₃)-2-oxo-5-(trifluoromethyl)pyrrolidin-3- |
| | yl)-2-(1-(1-methylpyrrolidin-2-yl)ethoxy)-5,6-dihydro-4H-benzo[de][1,6]naphthyridin- |
| | 8-yl)-7-fluorobenzo[b]thiophene-3-carbonitrile |
| 54 | 2-amino-4-(7-chloro-2-((2-(difluoromethylene)tetrahydro-1H-pyrrolizin-7a(5H)- |
| | yl)methoxy)-9-fluoro-4-(1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 55 | 2-amino-4-(7-chloro-9-fluoro-4-(1-(methyl-d₃)-2-oxo-5-(trifluoromethyl)pyrrolidin-3- |
| | yl)-2-((1-methylpyrrolidin-2-yl)methoxy)-5,6-dihydro-4H-benzo[de][1,6]naphthyridin- |
| | 8-yl)-7-fluorobenzo[b]thiophene-3-carbonitrile |
| 56 | 2-amino-4-(7-chloro-9-fluoro-4-(1-(methyl-d₃)-2-oxo-5-(trifluoromethyl)pyrrolidin-3- |
| | yl)-2-(1-(1-methylpyrrolidin-2-yl)ethoxy)-5,6-dihydro-4H-benzo[de][1,6]naphthyridin- |
| | 8-yl)-7-fluorobenzo[b]thiophene-3-carbonitrile |
| 57 | 2-amino-4-(7-chloro-9-fluoro-4-(1-(methyl-d₃)-2-oxo-5-(trifluoromethyl)pyrrolidin-3- |
| | yl)-2-(1H-1,2,4-triazol-1-yl)-5,6-dihydro-4H-benzo[de][1,6]naphthyridin-8-yl)-7- |
| | fluorobenzo[b]thiophene-3-carbonitrile |
| 58 | 8-(2-amino-3-cyano-7-fluorobenzo[b]thiophen-4-yl)-7-chloro-9-fluoro-4-(1-(methyl- |
| | d₃)-2-oxo-5-(trifluoromethyl)pyrrolidin-3-yl)-N-(tetrahydro-2H-pyran-4-yl)-5,6- |
| | dihydro-4H-benzo[de][1,6]naphthyridine-2-carboxamide |
| 59 | 8-(2-amino-3-cyano-7-fluorobenzo[b]thiophen-4-yl)-7-chloro-9-fluoro-4-(1-(methyl- |
| | d₃)-2-oxo-5-(trifluoromethyl)pyrrolidin-3-yl)-5,6-dihydro-4H- |
| | benzo[de][1,6]naphthyridine-2-carbonitrile |
| 60 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 61 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 62 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 63 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 64 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 65 | 2-amino-7-fluoro-4-(9-fluoro-2-methyl-4-(1-(methyl-d₃)-2-oxo-5- |
| | (trifluoromethyl) pyrrolidin-3-yl)-7-(trifluoromethyl)-5,6-dihydro-4H-benzo[de] |
| | [1,6]naphthyridin-8-yl)benzo[b]thiophene-3-carbonitrile |
| 66 | 2-amino-4-(7-(difluoromethyl)-9-fluoro-2-methyl-4-(1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 67 | 2-amino-4-(7-chloro-3,9-difluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 68 | 2-amino-7-fluoro-4-(9-fluoro-2,7-dimethyl-4-(1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 69 | 2-amino-4-(2-(difluoromethyl)-9-fluoro-7-methyl-4-(1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 70 | 2-amino-4-(2-(3-(dimethylamino)-3-methylazetidin-1-yl)-9-fluoro-7-methyl-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 71 | 2-amino-4-(2-(3-(dimethylamino)azetidin-1-yl)-9-fluoro-7-methyl-4-(1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 72 | 2-amino-4-(7-chloro-9-fluoro-4-(5-fluoro-1-methyl-2-oxopiperidin-3-yl)-2-methyl- |
| | 5,6-dihydro-4H-benzo[de][1,6]naphthyridin-8-yl)-7-fluorobenzo[b]thiophene-3- |
| | carbonitrile |
| 73 | 2-amino-4-(7-chloro-9-fluoro-2-(3-hydroxy-3-methylbut-1-yn-1-yl)-4-(1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 74 | 2-amino-4-(7-chloro-2-(3-(dimethylamino)prop-1-yn-1-yl)-9-fluoro-4-(1-(methyl-d₃)- |
| | 2-oxo-5-(trifluoromethyl)pyrrolidin-3-yl)-5,6-dihydro-4H-benzo[de] [1,6]naphthyridin- |
| | 8-yl)-7-fluorobenzo[b]thiophene-3-carbonitrile |
| 75 | 2-amino-4-(7-chloro-9-fluoro-2-methyl-4-(5-oxooctahydroindolizin-6-yl)-5,6-dihydro- |
| | 4H-benzo[de][1,6]naphthyridin-8-yl)-7-fluorobenzo[b]thiophene-3-carbonitrile |
| 76 | 2-amino-4-(7-chloro-9-fluoro-4-(1-(methyl-d₃)-2-oxo-5-(trifluoromethyl)pyrrolidin-3- |
| | yl)-2-(1-methyl-1,6-diazaspiro[3.3]heptan-6-yl)-5,6-dihydro-4H- |
| | benzo[de][1,6]naphthyridin-8-yl)-7-fluorobenzo[b]thiophene-3-carbonitrile |
| 77 | 2-amino-4-(7-chloro-2-(3-(dimethylamino)-4-fluoropyrrolidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 78 | 2-amino-4-(7-chloro-9-fluoro-4-(1-(methyl-d₃)-2-oxo-5-(trifluoromethyl)pyrrolidin-3- |
| | yl)-2-(5-methyl-2,5-diazabicyclo[2.2.1]heptan-2-yl)-5,6-dihydro-4H- |
| | benzo[de][1,6]naphthyridin-8-yl)-7-fluorobenzo[b]thiophene-3-carbonitrile |
| 79 | 2-amino-4-(7-chloro-9-fluoro-2-methyl-4-(1-(methyl-d₃)-2-oxo-5-phenylpyrrolidin-3- |
| | yl)-5,6-dihydro-4H-benzo[de][1,6]naphthyridin-8-yl)-7-fluorobenzo[b]thiophene-3- |
| | carbonitrile |
| 80 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 81 | 2-amino-4-(7-chloro-2-((dimethyl(oxo)-λ⁶-sulfaneylidene)amino)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 82 | 2-amino-4-(7-chloro-2-(3-(dimethylamino)pyrrolidin-1-yl)-9-fluoro-4-(1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 83 | 2-amino-4-(7-chloro-2-(3-(dimethylamino)-3-methylpyrrolidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 84 | 2-amino-4-(7-chloro-9-fluoro-4-(1-(methyl-d₃)-2-oxo-5-(trifluoromethyl)pyrrolidin-3- |
| | yl)-2-(1-methylhexahydropyrrolo[3,4-b]pyrrol-5(1H)-yl)-5,6-dihydro-4H- |
| | benzo[de][1,6]naphthyridin-8-yl)-7-fluorobenzo[b]thiophene-3-carbonitrile |
| 85 | 2-amino-4-(7-chloro-2-(3-cyclopropyl-3-hydroxyazetidin-1-yl)-9-fluoro-4-(1-(methyl- |
| | d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 86 | 2-amino-4-(7-chloro-2-(3-(dimethylamino)-3-ethylpyrrolidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 87 | 2-amino-4-(7-chloro-2-(3-cyclopropyl-3-(dimethylamino)pyrrolidin-1-yl)-9-fluoro-4- |
| | (1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 88 | 2-amino-4-(7-chloro-9-fluoro-2-methyl-4-(1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 89 | 8-(2-amino-3-cyano-7-fluorobenzo[b]thiophen-4-yl)-7-chloro-9-fluoro-2-methyl-4-(1- |
| | (methyl-d₃)-2-oxo-5-(trifluoromethyl)pyrrolidin-3-yl)-5,6-dihydro-4H- |
| | benzo[de][1,6]naphthyridine-3-carbonitrile |
| 90 | 8-(2-amino-3-cyano-7-fluorobenzo[b]thiophen-4-yl)-9-fluoro-2,7-dimethyl-4-(1- |
| | (methyl-d₃)-2-oxo-5-(trifluoromethyl)pyrrolidin-3-yl)-5,6-dihydro-4H- |
| | benzo[de][1,6]naphthyridine-3-carbonitrile |
| 91 | 2-amino-4-(2-(azetidin-1-ylmethyl)-7-chloro-9-fluoro-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 92 | 2-amino-4-(7-chloro-9-fluoro-4-(1-(methyl-d₃)-2-oxo-5-(trifluoromethyl)pyrrolidin-3- |
| | yl)-2-(trifluoromethyl)-5,6-dihydro-4H-benzo[de][1,6]naphthyridin-8-yl)-7- |
| | fluorobenzo[b]thiophene-3-carbonitrile |
| 93 | 2-amino-4-(7-chloro-9-fluoro-2-methyl-4-(1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 94 | 2-amino-4-(7-chloro-2-(3-(dimethylamino)-4-methoxypyrrolidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 95 | 2-amino-4-(7-chloro-2-(((1, 1-dioxidothietan-3-yl)oxy)methyl)-9-fluoro-4-(1-(methyl- |
| | d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 96 | 2-amino-4-(7-chloro-9-fluoro-4-(1-(methyl-d₃)-2-oxo-5-(trifluoromethyl)pyrrolidin-3- |
| | yl)-2-((2-(methylsulfonyl)ethoxy)methyl)-5,6-dihydro-4H- |
| | benzo[de][1,6]naphthyridin-8-yl)-7-fluorobenzo[b]thiophene-3-carbonitrile |
| 97 | 2-amino-4-(7-chloro-9-fluoro-2-((methoxy-d3)methyl)-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 98 | 2-amino-4-(7-chloro-2-((difluoromethoxy)methyl)-9-fluoro-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 99 | 2-amino-4-(7-chloro-2-((2-cyanoethoxy)methyl)-9-fluoro-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 100 | 2-amino-4-(7-chloro-9-fluoro-2-(hydroxymethyl)-4-(1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 101 | 2-amino-4-(7-chloro-2-(3-(dimethylamino)cyclobutyl)-9-fluoro-4-(1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 102 | 2-amino-4-(7-chloro-2-(3-(dimethylamino)-4-methoxypyrrolidin-1-yl)-9-fluoro-4-(1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 103 | 2-amino-4-(7-chloro-2-(4-(dimethylamino)-2-azabicyclo[2.1.1]hexan-2-yl)-9-fluoro- |
| | 4-(1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 104 | 2-amino-4-(7-chloro-4-(1-ethyl-2-oxopyrrolidin-3-yl)-9-fluoro-2-methyl-5,6-dihydro- |
| | 4H-benzo[de][1,6]naphthyridin-8-yl)-7-fluorobenzo[b]thiophene-3-carbonitrile |
| 105 | 2-amino-4-(7-chloro-9-fluoro-4-(1-(isothiazol-4-yl)ethyl)-2-methyl-5,6-dihydro-4H- |
| | benzo[de][1,6]naphthyridin-8-yl)-7-fluorobenzo[b]thiophene-3-carbonitrile |
| 106 | 2-amino-4-(7-chloro-9-fluoro-2-methyl-4-(5-methyl-1-(methyl-d₃)-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 107 | 2-amino-4-(7-chloro-4-(1,4-dimethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3- |
| | carbonitrile |
| 108 | 2-amino-4-(7-chloro-4-(1,5-dimethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3- |
| | carbonitrile |
| 109 | 2-amino-4-(7-chloro-9-fluoro-2-methyl-4-(2-oxo-1-(trifluoromethyl)pyrrolidin-3-yl)- |
| | 5,6-dihydro-4H-benzo[de][1,6]naphthyridin-8-yl)-7-fluorobenzo[b]thiophene-3- |
| | carbonitrile |

In other embodiments, the compound of Formula (I) is selected from the compounds in Table 2, and pharmaceutically acceptable salts thereof.

TABLE 2

| Entry | Compound Name |
|---|---|
| 1 | 2-amino-4-(4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 2 | 2-amino-4-(4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 3 | 2-amino-4-((Rₐ)-4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 4 | 2-amino-4-((Sₐ)-4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 5 | 2-amino-4-((Sₐ)-4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 6 | 2-amino-4-((Rₐ)-4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 7 | 4-(4-((R)-1-(2-aminopyridin-3-yl)ethyl)-7-chloro-9-fluoro-2-methyl-5,6-dihydro-4H- |
| | benzo[de][1,6]naphthyridin-8-yl)-7-fluorobenzo[d]thiazol-2-amine |
| 8 | 4-(4-((S)-1-(2-aminopyridin-3-yl)ethyl)-7-chloro-9-fluoro-2-methyl-5,6-dihydro-4H- |
| | benzo[de][1,6]naphthyridin-8-yl)-7-fluorobenzo[d]thiazol-2-amine |
| 9 | 4-((Rₐ)-4-((R)-1-(2-aminopyridin-3-yl)ethyl)-7-chloro-9-fluoro-2-methyl-5,6-dihydro- |
| | 4H-benzo[de][1,6]naphthyridin-8-yl)-7-fluorobenzo[d]thiazol-2-amine |
| 10 | 4-((Sₐ)-4-((S)-1-(2-aminopyridin-3-yl)ethyl)-7-chloro-9-fluoro-2-methyl-5,6-dihydro- |
| | 4H-benzo[de][1,6]naphthyridin-8-yl)-7-fluorobenzo[d]thiazol-2-amine |
| 11 | 4-((Rₐ)-4-((S)-1-(2-aminopyridin-3-yl)ethyl)-7-chloro-9-fluoro-2-methyl-5,6-dihydro- |
| | 4H-benzo[de][1,6]naphthyridin-8-yl)-7-fluorobenzo[d]thiazol-2-amine |
| 12 | 4-((Sₐ)-4-((R)-1-(2-aminopyridin-3-yl)ethyl)-7-chloro-9-fluoro-2-methyl-5,6-dihydro- |
| | 4H-benzo[de][1,6]naphthyridin-8-yl)-7-fluorobenzo[d]thiazol-2-amine |
| 13 | 8-(4-((R)-1-(2-aminopyridin-3-yl)ethyl)-7-chloro-9-fluoro-2-methyl-5,6-dihydro-4H- |
| | benzo[de][1,6]naphthyridin-8-yl)-1-naphthonitrile |
| 14 | 8-(4-((S)-1-(2-aminopyridin-3-yl)ethyl)-7-chloro-9-fluoro-2-methyl-5,6-dihydro-4H- |
| | benzo[de][1,6]naphthyridin-8-yl)-1-naphthonitrile |
| 15 | 8-((Rₐ)-4-((R)-1-(2-aminopyridin-3-yl)ethyl)-7-chloro-9-fluoro-2-methyl-5,6-dihydro- |
| | 4H-benzo[de][1,6]naphthyridin-8-yl)-1-naphthonitrile |
| 16 | 8-((Sₐ)-4-((S)-1-(2-aminopyridin-3-yl)ethyl)-7-chloro-9-fluoro-2-methyl-5,6-dihydro- |
| | 4H-benzo[de][1,6]naphthyridin-8-yl)-1-naphthonitrile |
| 17 | 8-((Rₐ)-4-((S)-1-(2-aminopyridin-3-yl)ethyl)-7-chloro-9-fluoro-2-methyl-5,6-dihydro- |
| | 4H-benzo[de][1,6]naphthyridin-8-yl)-1-naphthonitrile |
| 18 | 8-((Sₐ)-4-((R)-1-(2-aminopyridin-3-yl)ethyl)-7-chloro-9-fluoro-2-methyl-5,6-dihydro- |
| | 4H-benzo[de][1,6]naphthyridin-8-yl)-1-naphthonitrile |
| 19 | 3-(1-((R)-7-chloro-8-(5,6-dimethyl-1H-indazol-4-yl)-9-fluoro-2-methyl-5,6-dihydro- |
| | 4H-benzo[de][1,6]naphthyridin-4-yl)ethyl)pyridin-2-amine |
| 20 | 3-(1-((S)-7-chloro-8-(5,6-dimethyl-1H-indazol-4-yl)-9-fluoro-2-methyl-5,6-dihydro- |
| | 4H-benzo[de][1,6]naphthyridin-4-yl)ethyl)pyridin-2-amine |
| 21 | 3-((Rₐ)-1-((R)-7-chloro-8-(5,6-dimethyl-1H-indazol-4-yl)-9-fluoro-2-methyl-5,6- |
| | dihydro-4H-benzo[de][1,6]naphthyridin-4-yl)ethyl)pyridin-2-amine |
| 22 | 3-((Sₐ)-1-((S)-7-chloro-8-(5,6-dimethyl-1H-indazol-4-yl)-9-fluoro-2-methyl-5,6- |
| | dihydro-4H-benzo[de][1,6]naphthyridin-4-yl)ethyl)pyridin-2-amine |
| 23 | 3-((Rₐ)-1-((S)-7-chloro-8-(5,6-dimethyl-1H-indazol-4-yl)-9-fluoro-2-methyl-5,6- |
| | dihydro-4H-benzo[de][1,6]naphthyridin-4-yl)ethyl)pyridin-2-amine |
| 24 | 3-((Sₐ)-1-((R)-7-chloro-8-(5,6-dimethyl-1H-indazol-4-yl)-9-fluoro-2-methyl-5,6- |
| | dihydro-4H-benzo[de][1,6]naphthyridin-4-yl)ethyl)pyridin-2-amine. |
| 25 | 2-amino-4-(7-chloro-9-fluoro-2-methyl-4-((R)-1-(pyridin-3-yl)ethyl)-5,6-dihydro-4H- |
| | benzo[de][1,6]naphthyridin-8-yl)-7-fluorobenzo[b]thiophene-3-carbonitrile |
| 26 | 2-amino-4-(7-chloro-9-fluoro-2-methyl-4-((R)-1-(pyridin-3-yl)ethyl)-5,6-dihydro-4H- |
| | benzo[de][1,6]naphthyridin-8-yl)-7-fluorobenzo[b]thiophene-3-carbonitrile |
| 27 | 2-amino-(Rₐ)-4-(7-chloro-9-fluoro-2-methyl-4-((R)-1-(pyridin-3-yl)ethyl)-5,6-dihydro- |
| | 4H-benzo[de][1,6]naphthyridin-8-yl)-7-fluorobenzo[b]thiophene-3-carbonitrile |
| 28 | 2-amino-(Sₐ)-4-(7-chloro-9-fluoro-2-methyl-4-((R)-1-(pyridin-3-yl)ethyl)-5,6-dihydro- |
| | 4H-benzo[de][1,6]naphthyridin-8-yl)-7-fluorobenzo[b]thiophene-3-carbonitrile |
| 29 | 2-amino-(Rₐ)-4-(7-chloro-9-fluoro-2-methyl-4-((S)-1-(pyridin-3-yl)ethyl)-5,6-dihydro- |
| | 4H-benzo[de][1,6]naphthyridin-8-yl)-7-fluorobenzo[b]thiophene-3-carbonitrile |
| 30 | 2-amino-(Sₐ)-4-(7-chloro-9-fluoro-2-methyl-4-((S)-1-(pyridin-3-yl)ethyl)-5,6-dihydro- |
| | 4H-benzo[de][1,6]naphthyridin-8-yl)-7-fluorobenzo[b]thiophene-3-carbonitrile |
| 31 | 2-amino-4-(4-((R)-1-(3-aminopyrazin-2-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 32 | 2-amino-4-(4-((R)-1-(3-aminopyrazin-2-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 33 | 2-amino-(Rₐ)-4-(4-((R)-1-(3-aminopyrazin-2-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 34 | 2-amino-(Sₐ)-4-(4-((R)-1-(3-aminopyrazin-2-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 35 | 2-amino-(Rₐ)-4-(4-((S)-1-(3-aminopyrazin-2-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 36 | 2-amino-(Sₐ)-4-(4-((S)-1-(3-aminopyrazin-2-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 37 | 2-amino-4-(4-((R)-1-(3-aminopyridazin-4-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 38 | 2-amino-4-(4-((R)-1-(3-aminopyridazin-4-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 39 | 2-amino-4-((Rₐ)-4-((R)-1-(3-aminopyridazin-4-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 40 | 2-amino-4-((Sₐ)-4-((S)-1-(3-aminopyridazin-4-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 41 | 2-amino-4-((Sₐ)-4-((R)-1-(3-aminopyridazin-4-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 42 | 2-amino-4-((Rₐ)-4-((S)-1-(3-aminopyridazin-4-yl)ethyl)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 43 | 2-amino-4-(4-((R)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 44 | 2-amino-4-(4-((S)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 45 | 2-amino-4-((Rₐ)-4-((R)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 46 | 2-amino-4-((Sₐ)-4-((S)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 47 | 2-amino-4-((Sₐ)-4-((R)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 48 | 2-amino-4-((Rₐ)-4-((S)-1-(5-aminopyridin-3-yl)ethyl)-7-chloro-9-fluoro-2-methyl-5,6- |
| | dihydro-4H-benzo[de][1,6]naphthyridin-8-yl)-7-fluorobenzo[b]thiophene-3- |
| | carbonitrile |
| 49 | 2-amino-4-(7-chloro-9-fluoro-2-methyl-4-((R)-1-(1-methyl-1H-pyrazol-5-yl)ethyl)- |
| | 5,6-dihydro-4H-benzo[de][1,6]naphthyridin-8-yl)-7-fluorobenzo[b]thiophene-3- |
| | carbonitrile |
| 50 | 2-amino-4-(7-chloro-9-fluoro-2-methyl-4-((S)-1-(1-methyl-1H-pyrazol-5-yl)ethyl)- |
| | 5,6-dihydro-4H-benzo[de][1,6]naphthyridin-8-yl)-7-fluorobenzo[b]thiophene-3- |
| | carbonitrile |
| 51 | 2-amino-4-((Rₐ)-7-chloro-9-fluoro-2-methyl-4-((R)-1-(1-methyl-1H-pyrazol-5- |
| | yl)ethyl)-5,6-dihydro-4H-benzo[de][1,6]naphthyridin-8-yl)-7- |
| | fluorobenzo[b]thiophene-3-carbonitrile |
| 52 | 2-amino-4-((Sₐ)-7-chloro-9-fluoro-2-methyl-4-((S)-1-(1-methyl-1H-pyrazol-5- |
| | yl)ethyl)-5,6-dihydro-4H-benzo[de][1,6]naphthyridin-8-yl)-7- |
| | fluorobenzo[b]thiophene-3-carbonitrile |
| 53 | 2-amino-4-((Sₐ)-7-chloro-9-fluoro-2-methyl-4-((R)-1-(1-methyl-1H-pyrazol-5- |
| | yl)ethyl)-5,6-dihydro-4H-benzo[de][1,6]naphthyridin-8-yl)-7- |
| | fluorobenzo[b]thiophene-3-carbonitrile |
| 54 | 2-amino-4-((Rₐ)-7-chloro-9-fluoro-2-methyl-4-((S)-1-(1-methyl-1H-pyrazol-5- |
| | yl)ethyl)-5,6-dihydro-4H-benzo[de][1,6]naphthyridin-8-yl)-7- |
| | fluorobenzo[b]thiophene-3-carbonitrile |
| 55 | 2-amino-4-(4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 56 | 2-amino-4-(4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 57 | 2-amino-4-((Rₐ)-4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 58 | 2-amino-4-((Sₐ)-4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 59 | 2-amino-4-((Sₐ)-4-((R)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 60 | 2-amino-4-((Rₐ)-4-((S)-1-(2-aminopyridin-3-yl)ethyl)-7-chloro-9-fluoro-2-methyl-5,6- |
| | dihydro-4H-benzo[de][1,6]naphthyridin-8-yl)-7-fluorothieno[3,2-c]pyridine-3- |
| | carbonitrile |
| 61 | 2-amino-4-((S)-4-((R)-1-(2-aminopyridin-3-yl)ethyl)-7-chloro-9-fluoro-2,6-dimethyl- |
| | 5,6-dihydro-4H-benzo[de][1,6]naphthyridin-8-yl)-7-fluorobenzo[b]thiophene-3- |
| | carbonitrile |
| 62 | 2-amino-4-((R)-4-((R)-1-(2-aminopyridin-3-yl)ethyl)-7-chloro-9-fluoro-2,6-dimethyl- |
| | 5,6-dihydro-4H-benzo[de][1,6]naphthyridin-8-yl)-7-fluorobenzo[b]thiophene-3- |
| | carbonitrile |
| 63 | 2-amino-(Rₐ)-4-((S)-4-((R)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 64 | 2-amino-(Sₐ)-4-((S)-4-((R)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 65 | 2-amino-(Rₐ)-4-((R)-4-((R)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 66 | 2-amino-(Sₐ)-4-((R)-4-((R)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 67 | 2-amino-4-((S)-4-((S)-1-(2-aminopyridin-3-yl)ethyl)-7-chloro-9-fluoro-2,6-dimethyl- |
| | 5,6-dihydro-4H-benzo[de][1,6]naphthyridin-8-yl)-7-fluorobenzo[b]thiophene-3- |
| | carbonitrile |
| 68 | 2-amino-4-((R)-4-((S)-1-(2-aminopyridin-3-yl)ethyl)-7-chloro-9-fluoro-2,6-dimethyl- |
| | 5,6-dihydro-4H-benzo[de][1,6]naphthyridin-8-yl)-7-fluorobenzo[b]thiophene-3- |
| | carbonitrile |
| 69 | 2-amino-(Rₐ)-4-((S)-4-((S)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 70 | 2-amino-(Sₐ)-4-((S)-4-((S)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 71 | 2-amino-(Rₐ)-4-((R)-4-((S)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 72 | 2-amino-(Sₐ)-4-((R)-4-((S)-1-(2-aminopyridin-3-yl)ethyl)-7-chloro-9-fluoro-2,6- |
| | dimethyl-5,6-dihydro-4H-benzo[de][1,6]naphthyridin-8-yl)-7- |
| | fluorobenzo[b]thiophene-3-carbonitrile |
| 73 | 2-amino-4-((Rₐ)-4-((R)-1-(2-aminopyridin-3-yl)ethyl-1-d)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 74 | 2-amino-4-((Sₐ)-4-((S)-1-(2-aminopyridin-3-yl)ethyl-1-d)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 75 | 2-amino-4-((Rₐ)-4-((S)-1-(2-aminopyridin-3-yl)ethyl-1-d)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 76 | 2-amino-4-((Sₐ)-4-((R)-1-(2-aminopyridin-3-yl)ethyl-1-d)-7-chloro-9-fluoro-2-methyl- |
| | 5,6-dihydro-4H-benzo[de][1,6]naphthyridin-8-yl)-7-fluorobenzo[b]thiophene-3- |
| | carbonitrile |
| 77 | 2-amino-4-((Rₐ)-4-((R)-1-(2-amino-5-fluoropyridin-3-yl)ethyl-1-d)-7-chloro-9-fluoro- |
| | 2-methyl-5,6-dihydro-4H-benzo[de][1,6]naphthyridin-8-yl)-7- |
| | fluorobenzo[b]thiophene-3-carbonitrile |
| 78 | 2-amino-4-((Sₐ)-4-((S)-1-(2-amino-5-fluoropyridin-3-yl)ethyl-1-d)-7-chloro-9-fluoro- |
| | 2-methyl-5,6-dihydro-4H-benzo[de][1,6]naphthyridin-8-yl)-7- |
| | fluorobenzo[b]thiophene-3-carbonitrile |
| 79 | 2-amino-4-((Rₐ)-4-((S)-1-(2-amino-5-fluoropyridin-3-yl)ethyl-1-d)-7-chloro-9-fluoro- |
| | 2-methyl-5,6-dihydro-4H-benzo[de][1,6]naphthyridin-8-yl)-7- |
| | fluorobenzo[b]thiophene-3-carbonitrile |
| 80 | 2-amino-4-((Sₐ)-4-((R)-1-(2-amino-5-fluoropyridin-3-yl)ethyl-1-d)-7-chloro-9-fluoro- |
| | 2-methyl-5,6-dihydro-4H-benzo[de][1,6]naphthyridin-8-yl)-7- |
| | fluorobenzo[b]thiophene-3-carbonitrile |
| 81 | 2-amino-4-((Rₐ)-4-((R)-(2-aminopyridin-3-yl)(cyclopropyl)methyl-d)-7-chloro-9- |
| | fluoro-2-methyl-5,6-dihydro-4H-benzo[de][1,6]naphthyridin-8-yl)-7- |
| | fluorobenzo[b]thiophene-3-carbonitrile |
| 82 | 2-amino-4-((Sₐ)-4-((S)-(2-aminopyridin-3-yl)(cyclopropyl)methyl-d)-7-chloro-9- |
| | fluoro-2-methyl-5,6-dihydro-4H-benzo[de][1,6]naphthyridin-8-yl)-7- |
| | fluorobenzo[b]thiophene-3-carbonitrile |
| 83 | 2-amino-4-((Rₐ)-4-((S)-(2-aminopyridin-3-yl)(cyclopropyl)methyl-d)-7-chloro-9- |
| | fluoro-2-methyl-5,6-dihydro-4H-benzo[de][1,6]naphthyridin-8-yl)-7- |
| | fluorobenzo[b]thiophene-3-carbonitrile |
| 84 | 2-amino-4-((Sₐ)-4-((R)-(2-aminopyridin-3-yl)(cyclopropyl)methyl-d)-7-chloro-9- |
| | fluoro-2-methyl-5,6-dihydro-4H-benzo[de][1,6]naphthyridin-8-yl)-7- |
| | fluorobenzo[b]thiophene-3-carbonitrile |
| 85 | 2-amino-4-(Rₐ)-(7-chloro-9-fluoro-2-methyl-4-((R)-1-methyl-2-oxopyrrolidin-3-yl)- |
| | 5,6-dihydro-4H-benzo[de][1,6]naphthyridin-8-yl)-7-fluorobenzo[b]thiophene-3- |
| | carbonitrile |
| 86 | 2-amino-4-(Sₐ)-(7-chloro-9-fluoro-2-methyl-4-((S)-1-methyl-2-oxopyrrolidin-3-yl)- |
| | 5,6-dihydro-4H-benzo[de][1,6]naphthyridin-8-yl)-7-fluorobenzo[b]thiophene-3- |
| | carbonitrile |
| 87 | 2-amino-4-(Rₐ)-(7-chloro-9-fluoro-2-methyl-4-((S)-1-methyl-2-oxopyrrolidin-3-yl)- |
| | 5,6-dihydro-4H-benzo[de][1,6]naphthyridin-8-yl)-7-fluorobenzo[b]thiophene-3- |
| | carbonitrile |
| 88 | 2-amino-4-(Sₐ)-(7-chloro-9-fluoro-2-methyl-4-((R)-1-methyl-2-oxopyrrolidin-3-yl)- |
| | 5,6-dihydro-4H-benzo[de][1,6]naphthyridin-8-yl)-7-fluorobenzo[b]thiophene-3- |
| | carbonitrile |
| 89 | 2-amino-4-(Rₐ)-(7-chloro-4-(1-cyclopropyl-2-oxopyrrolidin-3-yl)-9-fluoro-2-methyl- |
| | 5,6-dihydro-4H-benzo[de][1,6]naphthyridin-8-yl)-7-fluorobenzo[b]thiophene-3- |
| | carbonitrile |
| 90 | 2-amino-4-(Sₐ)-(7-chloro-4-(1-cyclopropyl-2-oxopyrrolidin-3-yl)-9-fluoro-2-methyl- |
| | 5,6-dihydro-4H-benzo[de][1,6]naphthyridin-8-yl)-7-fluorobenzo[b]thiophene-3- |
| | carbonitrile |
| 91 | 2-amino-4-(Rₐ)-(7-chloro-4-((R)-1-cyclopropyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 92 | 2-amino-4-(Sₐ)-(7-chloro-4-((S)-1-cyclopropyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 93 | 2-amino-4-(Rₐ)-(7-chloro-4-((S)-1-cyclopropyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 94 | 2-amino-4-(Sₐ)-(7-chloro-4-((R)-1-cyclopropyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 95 | 2-amino-4-(Rₐ)-(7-chloro-9-fluoro-2-methyl-4-(2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 96 | 2-amino-4-(Sₐ)-(7-chloro-9-fluoro-2-methyl-4-(2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 97 | 2-amino-4-(Rₐ)-(7-chloro-9-fluoro-2-methyl-4-((R)-2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 98 | 2-amino-4-(Sₐ)-(7-chloro-9-fluoro-2-methyl-4-((S)-2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 99 | 2-amino-4-(Rₐ)-(7-chloro-9-fluoro-2-methyl-4-((S)-2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 100 | 2-amino-4-(Sₐ)-(7-chloro-9-fluoro-2-methyl-4-((R)-2-oxo-1-(2,2,2-trifluoroethyl) |
| | pyrrolidin-3-yl)-5,6-dihydro-4H-benzo[de][1,6]naphthyridin-8-yl)-7- |
| | fluorobenzo[b]thiophene-3-carbonitrile |
| 101 | 2-amino-4-(Rₐ)-(7-chloro-9-fluoro-2-methyl-4-(2-oxo-1-phenylpyrrolidin-3-yl)-5,6- |
| | dihydro-4H-benzo[de][1,6]naphthyridin-8-yl)-7-fluorobenzo[b]thiophene-3- |
| | carbonitrile |
| 102 | 2-amino-4-(Sₐ)-(7-chloro-9-fluoro-2-methyl-4-(2-oxo-1-phenylpyrrolidin-3-yl)-5,6- |
| | dihydro-4H-benzo[de][1,6]naphthyridin-8-yl)-7-fluorobenzo[b]thiophene-3- |
| | carbonitrile |
| 103 | 2-amino-4-(Rₐ)-(7-chloro-9-fluoro-2-methyl-4-((R)-2-oxo-1-phenylpyrrolidin-3-yl)- |
| | 5,6-dihydro-4H-benzo[de][1,6]naphthyridin-8-yl)-7-fluorobenzo[b]thiophene-3- |
| | carbonitrile |
| 104 | 2-amino-4-(Sₐ)-(7-chloro-9-fluoro-2-methyl-4-((S)-2-oxo-1-phenylpyrrolidin-3-yl)- |
| | 5,6-dihydro-4H-benzo[de][1,6]naphthyridin-8-yl)-7-fluorobenzo[b]thiophene-3- |
| | carbonitrile |
| 105 | 2-amino-4-(Rₐ)-(7-chloro-9-fluoro-2-methyl-4-((S)-2-oxo-1-phenylpyrrolidin-3-yl)- |
| | 5,6-dihydro-4H-benzo[de][1,6]naphthyridin-8-yl)-7-fluorobenzo[b]thiophene-3- |
| | carbonitrile |
| 106 | 2-amino-4-(Sₐ)-(7-chloro-9-fluoro-2-methyl-4-((R)-2-oxo-1-phenylpyrrolidin-3-yl)- |
| | 5,6-dihydro-4H-benzo[de][1,6]naphthyridin-8-yl)-7-fluorobenzo[b]thiophene-3- |
| | carbonitrile |
| 107 | 2-amino-4-(Rₐ)-(7-chloro-9-fluoro-2-methyl-4-(1-methyl-2-oxopiperidin-4-yl)-5,6- |
| | dihydro-4H-benzo[de][1,6]naphthyridin-8-yl)-7-fluorobenzo[b]thiophene-3- |
| | carbonitrile |
| 108 | 2-amino-4-(Sₐ)-(7-chloro-9-fluoro-2-methyl-4-(1-methyl-2-oxopiperidin-4-yl)-5,6- |
| | dihydro-4H-benzo[de][1,6]naphthyridin-8-yl)-7-fluorobenzo[b]thiophene-3- |
| | carbonitrile |
| 109 | 2-amino-4-(Rₐ)-(7-chloro-9-fluoro-2-methyl-4-((R)-1-methyl-2-oxopiperidin-4-yl)- |
| | 5,6-dihydro-4H-benzo[de][1,6]naphthyridin-8-yl)-7-fluorobenzo[b]thiophene-3- |
| | carbonitrile |
| 110 | 2-amino-4-(Sₐ)-(7-chloro-9-fluoro-2-methyl-4-((S)-1-methyl-2-oxopiperidin-4-yl)- |
| | 5,6-dihydro-4H-benzo[de][1,6]naphthyridin-8-yl)-7-fluorobenzo[b]thiophene-3- |
| | carbonitrile |
| 111 | 2-amino-4-(Rₐ)-(7-chloro-9-fluoro-2-methyl-4-((S)-1-methyl-2-oxopiperidin-4-yl)- |
| | 5,6-dihydro-4H-benzo[de][1,6]naphthyridin-8-yl)-7-fluorobenzo[b]thiophene-3- |
| | carbonitrile |
| 112 | 2-amino-4-(Sₐ)-(7-chloro-9-fluoro-2-methyl-4-((R)-1-methyl-2-oxopiperidin-4-yl)- |
| | 5,6-dihydro-4H-benzo[de][1,6]naphthyridin-8-yl)-7-fluorobenzo[b]thiophene-3- |
| | carbonitrile |
| 113 | 2-amino-4-(Rₐ)-(7-chloro-9-fluoro-2-methyl-4-((2R,8aR)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 114 | 2-amino-4-(Sₐ)-(7-chloro-9-fluoro-2-methyl-4-((2S,8aS)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 115 | 2-amino-4-(Rₐ)-(7-chloro-9-fluoro-2-methyl-4-((2S,8aR)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 116 | 2-amino-4-(Sₐ)-(7-chloro-9-fluoro-2-methyl-4-((2R,8aS)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 117 | 2-amino-4-(Rₐ)-(7-chloro-9-fluoro-2-methyl-4-((2R,8aS)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 118 | 2-amino-4-(Sₐ)-(7-chloro-9-fluoro-2-methyl-4-((2S,8aR)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 119 | 2-amino-4-(Rₐ)-(7-chloro-9-fluoro-2-methyl-4-((2S,8aS)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 120 | 2-amino-4-(Sₐ)-(7-chloro-9-fluoro-2-methyl-4-((2R,8aR)-3-oxooctahydroindolizin-2- |
| | yl)-5,6-dihydro-4H-benzo[de][1,6]naphthyridin-8-yl)-7-fluorobenzo[b]thiophene-3- |
| | carbonitrile |
| 121 | 2-amino-4-(Rₐ)-(7-chloro-9-fluoro-2-methyl-4-((R)-1-methyl-2-oxopiperidin-3-yl)- |
| | 5,6-dihydro-4H-benzo[de][1,6]naphthyridin-8-yl)-7-fluorobenzo[b]thiophene-3- |
| | carbonitrile |
| 122 | 2-amino-4-(Sₐ)-(7-chloro-9-fluoro-2-methyl-4-((S)-1-methyl-2-oxopiperidin-3-yl)- |
| | 5,6-dihydro-4H-benzo[de][1,6]naphthyridin-8-yl)-7-fluorobenzo[b]thiophene-3- |
| | carbonitrile |
| 123 | 2-amino-4-(Rₐ)-(7-chloro-9-fluoro-2-methyl-4-((S)-1-methyl-2-oxopiperidin-3-yl)- |
| | 5,6-dihydro-4H-benzo[de][1,6]naphthyridin-8-yl)-7-fluorobenzo[b]thiophene-3- |
| | carbonitrile |
| 124 | 2-amino-4-(Sₐ)-(7-chloro-9-fluoro-2-methyl-4-((R)-1-methyl-2-oxopiperidin-3-yl)- |
| | 5,6-dihydro-4H-benzo[de][1,6]naphthyridin-8-yl)-7-fluorobenzo[b]thiophene-3- |
| | carbonitrile |
| 125 | 2-amino-4-(Rₐ)-(7-chloro-9-fluoro-2-methyl-4-((R)-1-(1-methyl-1H-1,2,4-triazol-5- |
| | yl)ethyl)-5,6-dihydro-4H-benzo[de][1,6]naphthyridin-8-yl)-7- |
| | fluorobenzo[b]thiophene-3-carbonitrile |
| 126 | 2-amino-4-(Sₐ)-(7-chloro-9-fluoro-2-methyl-4-((S)-1-(1-methyl-1H-1,2,4-triazol-5- |
| | yl)ethyl)-5,6-dihydro-4H-benzo[de][1,6]naphthyridin-8-yl)-7- |
| | fluorobenzo[b]thiophene-3-carbonitrile |
| 127 | 2-amino-4-(Rₐ)-(7-chloro-9-fluoro-2-methyl-4-((S)-1-(1-methyl-1H-1,2,4-triazol-5- |
| | yl)ethyl)-5,6-dihydro-4H-benzo[de][1,6]naphthyridin-8-yl)-7- |
| | fluorobenzo[b]thiophene-3-carbonitrile |
| 128 | 2-amino-4-(Sₐ)-(7-chloro-9-fluoro-2-methyl-4-((R)-1-(1-methyl-1H-1,2,4-triazol-5- |
| | yl)ethyl)-5,6-dihydro-4H-benzo[de][1,6]naphthyridin-8-yl)-7- |
| | fluorobenzo[b]thiophene-3-carbonitrile |
| 129 | 2-amino-4-(Rₐ)-(7-chloro-9-fluoro-2-methyl-4-((R)-1-(methyl-d₃)-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 130 | 2-amino-4-(Sₐ)-(7-chloro-9-fluoro-2-methyl-4-((S)-1-(methyl-d₃)-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 131 | 2-amino-4-(Rₐ)-(7-chloro-9-fluoro-2-methyl-4-((S)-1-(methyl-d₃)-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 132 | 2-amino-4-(Sₐ)-(7-chloro-9-fluoro-2-methyl-4-((R)-1-(methyl-d₃)-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 133 | 4-(Rₐ)-(4-((R)-1-acetylpyrrolidin-3-yl)-7-chloro-9-fluoro-2-methyl-5,6-dihydro-4H- |
| | benzo[de][1,6]naphthyridin-8-yl)-2-amino-7-fluorobenzo[b]thiophene-3-carbonitrile |
| 134 | 4-(Sₐ)-(4-((S)-1-acetylpyrrolidin-3-yl)-7-chloro-9-fluoro-2-methyl-5,6-dihydro-4H- |
| | benzo[de][1,6]naphthyridin-8-yl)-2-amino-7-fluorobenzo[b]thiophene-3-carbonitrile |
| 135 | 4-(Rₐ)-(4-((S)-1-acetylpyrrolidin-3-yl)-7-chloro-9-fluoro-2-methyl-5,6-dihydro-4H- |
| | benzo[de][1,6]naphthyridin-8-yl)-2-amino-7-fluorobenzo[b]thiophene-3-carbonitrile |
| 136 | 4-(Sₐ)-(4-((R)-1-acetylpyrrolidin-3-yl)-7-chloro-9-fluoro-2-methyl-5,6-dihydro-4H- |
| | benzo[de][1,6]naphthyridin-8-yl)-2-amino-7-fluorobenzo[b]thiophene-3-carbonitrile |
| 137 | 2-amino-4-(Rₐ)-(7-chloro-9-fluoro-2-methyl-4-((3R,3aR,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 138 | 2-amino-4-(Sₐ)-(7-chloro-9-fluoro-2-methyl-4-((3S,3aS,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 139 | 2-amino-4-(Rₐ)-(7-chloro-9-fluoro-2-methyl-4-((3S,3aR,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 140 | 2-amino-4-(Sₐ)-(7-chloro-9-fluoro-2-methyl-4-((3R,3aS,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 141 | 2-amino-4-(Rₐ)-(7-chloro-9-fluoro-2-methyl-4-((3R,3aS,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 142 | 2-amino-4-(Sₐ)-(7-chloro-9-fluoro-2-methyl-4-((3S,3aR,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 143 | 2-amino-4-(Rₐ)-(7-chloro-9-fluoro-2-methyl-4-((3S,3aS,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 144 | 2-amino-4-(Sₐ)-(7-chloro-9-fluoro-2-methyl-4-((3R,3aR,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 145 | 2-amino-4-(Rₐ)-(7-chloro-9-fluoro-2-methyl-4-((3R,3aR,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 146 | 2-amino-4-(Sₐ)-(7-chloro-9-fluoro-2-methyl-4-((3S,3aS,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 147 | 2-amino-4-(Rₐ)-(7-chloro-9-fluoro-2-methyl-4-((3S,3aR,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 148 | 2-amino-4-(Sₐ)-(7-chloro-9-fluoro-2-methyl-4-((3R,3aS,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 149 | 2-amino-4-(Rₐ)-(7-chloro-9-fluoro-2-methyl-4-((3R,3aR,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 150 | 2-amino-4-(Sₐ)-(7-chloro-9-fluoro-2-methyl-4-((3S,3aS,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 151 | 2-amino-4-(Rₐ)-(7-chloro-9-fluoro-2-methyl-4-((3S,3aR,6aS)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 152 | 2-amino-4-(Sₐ)-(7-chloro-9-fluoro-2-methyl-4-((3R,3aS,6aR)-1-methyl-2- |
| | oxooctahydrocyclopenta[b]pyrrol-3-yl)-5,6-dihydro-4H-benzo[de][1,6]naphthyridin- |
| | 8-yl)-7-fluorobenzo[b]thiophene-3-carbonitrile |
| 153 | 2-amino-4-(Rₐ)-(7-chloro-9-fluoro-2-methyl-4-(2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 154 | 2-amino-4-(Sₐ)-(7-chloro-9-fluoro-2-methyl-4-(2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 155 | 2-amino-4-(Rₐ)-(7-chloro-9-fluoro-2-methyl-4-((1S,4R,5R)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 156 | 2-amino-4-(Sₐ)-(7-chloro-9-fluoro-2-methyl-4-((1R,4S,5S)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 157 | 2-amino-4-(Rₐ)-(7-chloro-9-fluoro-2-methyl-4-((1R,4R,5S)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 158 | 2-amino-4-(Sₐ)-(7-chloro-9-fluoro-2-methyl-4-((1S,4S,5R)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 159 | 2-amino-4-(Rₐ)-(7-chloro-9-fluoro-2-methyl-4-((1S,4S,5R)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 160 | 2-amino-4-(Sₐ)-(7-chloro-9-fluoro-2-methyl-4-((1R,4R,5S)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 161 | 2-amino-4-(Rₐ)-(7-chloro-9-fluoro-2-methyl-4-((1R,4S,5S)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 162 | 2-amino-4-(Sₐ)-(7-chloro-9-fluoro-2-methyl-4-((1S,4R,5R)-2-methyl-3-oxo-2- |
| | azabicyclo[3.1.0]hexan-4-yl)-5,6-dihydro-4H-benzo[de][1,6]naphthyridin-8-yl)-7- |
| | fluorobenzo[b]thiophene-3-carbonitrile |
| 163 | 2-amino-4-(Rₐ)-(7-chloro-9-fluoro-2-methyl-4-(2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 164 | 2-amino-4-(Sₐ)-(7-chloro-9-fluoro-2-methyl-4-(2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 165 | 2-amino-4-(Rₐ)-(7-chloro-9-fluoro-2-methyl-4-((1S,4R,5R)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 166 | 2-amino-4-(Sₐ)-(7-chloro-9-fluoro-2-methyl-4-((1R,4S,5S)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 167 | 2-amino-4-(Rₐ)-(7-chloro-9-fluoro-2-methyl-4-((1R,4R,5S)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 168 | 2-amino-4-(Sₐ)-(7-chloro-9-fluoro-2-methyl-4-((1S,4R,5R)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 169 | 2-amino-4-(Rₐ)-(7-chloro-9-fluoro-2-methyl-4-((1S,4S,5R)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 170 | 2-amino-4-(Sₐ)-(7-chloro-9-fluoro-2-methyl-4-((1R,4R,5S)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 171 | 2-amino-4-(Rₐ)-(7-chloro-9-fluoro-2-methyl-4-((1R,4S,5S)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 172 | 2-amino-4-(Sₐ)-(7-chloro-9-fluoro-2-methyl-4-((1S,4S,5S)-2-methyl-3-oxo-2- |
| | azabicyclo[3.2.0]heptan-4-yl)-5,6-dihydro-4H-benzo[de][1,6]naphthyridin-8-yl)-7- |
| | fluorobenzo[b]thiophene-3-carbonitrile |
| 173 | 2-amino-4-(Rₐ)-(7-chloro-2-(3-(dimethylamino)-3-methylazetidin-1-yl)-9-fluoro-4- |
| | ((R)-1-methyl-2-oxopyrrolidin-3-yl)-5,6-dihydro-4H-benzo[de][1,6]naphthyridin-8- |
| | yl)-7-fluorobenzo[b]thiophene-3-carbonitrile |
| 174 | 2-amino-4-(Sₐ)-(7-chloro-2-(3-(dimethylamino)-3-methylazetidin-1-yl)-9-fluoro-4- |
| | ((S)-1-methyl-2-oxopyrrolidin-3-yl)-5,6-dihydro-4H-benzo[de][1,6]naphthyridin-8-yl)- |
| | 7-fluorobenzo[b]thiophene-3-carbonitrile |
| 175 | 2-amino-4-(Rₐ)-(7-chloro-2-(3-(dimethylamino)-3-methylazetidin-1-yl)-9-fluoro-4- |
| | ((S)-1-methyl-2-oxopyrrolidin-3-yl)-5,6-dihydro-4H-benzo[de][1,6]naphthyridin-8-yl)- |
| | 7-fluorobenzo[b]thiophene-3-carbonitrile |
| 176 | 2-amino-4-(Sₐ)-(7-chloro-2-(3-(dimethylamino)-3-methylazetidin-1-yl)-9-fluoro-4- |
| | ((R)-1-methyl-2-oxopyrrolidin-3-yl)-5,6-dihydro-4H-benzo[de][1,6]naphthyridin-8- |
| | yl)-7-fluorobenzo[b]thiophene-3-carbonitrile |
| 177 | 8-(2-amino-3-cyano-7-fluorobenzo[b]thiophen-4-yl)-4-((R)-1-(2-aminopyridin-3- |
| | yl)ethyl)-7-chloro-9-fluoro-N-methyl-5,6-dihydro-4H-(Rₐ)- |
| | benzo[de][1,6]naphthyridine-2-carboxamide |
| 178 | 8-(2-amino-3-cyano-7-fluorobenzo[b]thiophen-4-yl)-4-((S)-1-(2-aminopyridin-3- |
| | yl)ethyl)-7-chloro-9-fluoro-N-methyl-5,6-dihydro-4H-(Sₐ)- |
| | benzo[de][1,6]naphthyridine-2-carboxamide |
| 179 | 8-(2-amino-3-cyano-7-fluorobenzo[b]thiophen-4-yl)-4-((S)-1-(2-aminopyridin-3- |
| | yl)ethyl)-7-chloro-9-fluoro-N-methyl-5,6-dihydro-4H-(Rₐ)- |
| | benzo[de][1,6]naphthyridine-2-carboxamide |
| 180 | 8-(2-amino-3-cyano-7-fluorobenzo[b]thiophen-4-yl)-4-((R)-1-(2-aminopyridin-3- |
| | yl)ethyl)-7-chloro-9-fluoro-N-methyl-5,6-dihydro-4H-(Sₐ)- |
| | benzo[de][1,6]naphthyridine-2-carboxamide |
| 181 | 2-amino-4-(Rₐ)-(4-((R)-1-(2-aminopyridin-3-yl)ethyl)-7-chloro-6,6,9-trifluoro-2- |
| | methyl-5,6-dihydro-4H-benzo[de][1,6]naphthyridin-8-yl)-7-fluorobenzo[b]thiophene- |
| | 3-carbonitrile |
| 182 | 2-amino-4-(Sₐ)-(4-((S)-1-(2-aminopyridin-3-yl)ethyl)-7-chloro-6,6,9-trifluoro-2- |
| | methyl-5,6-dihydro-4H-benzo[de][1,6]naphthyridin-8-yl)-7-fluorobenzo[b]thiophene- |
| | 3-carbonitrile |
| 183 | 2-amino-4-(Rₐ)-(4-((S)-1-(2-aminopyridin-3-yl)ethyl)-7-chloro-6,6,9-trifluoro-2- |
| | methyl-5,6-dihydro-4H-benzo[de][1,6]naphthyridin-8-yl)-7-fluorobenzo[b]thiophene- |
| | 3-carbonitrile |
| 184 | 2-amino-4-(Sₐ)-(4-((R)-1-(2-aminopyridin-3-yl)ethyl)-7-chloro-6,6,9-trifluoro-2- |
| | methyl-5,6-dihydro-4H-benzo[de][1,6]naphthyridin-8-yl)-7-fluorobenzo[b]thiophene- |
| | 3-carbonitrile |
| 185 | (3R)-3-(8-(2-amino-7-fluorobenzo[d]thiazol-4-yl)-7-chloro-9-fluoro-2-methyl-5,6- |
| | dihydro-4H-benzo[de][1,6]naphthyridin-4-yl)-1-methylpyrrolidin-2-one |
| 186 | (3S)-3-(8-(2-amino-7-fluorobenzo[d]thiazol-4-yl)-7-chloro-9-fluoro-2-methyl-5,6- |
| | dihydro-4H-benzo[de][1,6]naphthyridin-4-yl)-1-methylpyrrolidin-2-one |
| 187 | (3R)-3-(8-(Rₐ)-(2-amino-7-fluorobenzo[d]thiazol-4-yl)-7-chloro-9-fluoro-2-methyl- |
| | 5,6-dihydro-4H-benzo[de][1,6]naphthyridin-4-yl)-1-methylpyrrolidin-2-one |
| 188 | (3S)-3-(8-(Rₐ)-(2-amino-7-fluorobenzo[d]thiazol-4-yl)-7-chloro-9-fluoro-2-methyl- |
| | 5,6-dihydro-4H-benzo[de][1,6]naphthyridin-4-yl)-1-methylpyrrolidin-2-one |
| 189 | (3R)-3-(8-(Sₐ)-(2-amino-7-fluorobenzo[d]thiazol-4-yl)-7-chloro-9-fluoro-2-methyl- |
| | 5,6-dihydro-4H-benzo[de][1,6]naphthyridin-4-yl)-1-methylpyrrolidin-2-one |
| 190 | (3S)-3-(8-(Sₐ)-(2-amino-7-fluorobenzo[d]thiazol-4-yl)-7-chloro-9-fluoro-2-methyl- |
| | 5,6-dihydro-4H-benzo[de][1,6]naphthyridin-4-yl)-1-methylpyrrolidin-2-one |
| 191 | 8-(2-amino-3-cyano-7-fluorobenzo[b]thiophen-4-yl)-7-chloro-9-fluoro-N-methyl-4- |
| | ((R)-1-methyl-2-oxopyrrolidin-3-yl)-5,6-dihydro-4H-benzo[de][1,6]naphthyridine-2- |
| | carboxamide |
| 192 | 8-(2-amino-3-cyano-7-fluorobenzo[b]thiophen-4-yl)-7-chloro-9-fluoro-N-methyl-4- |
| | ((S)-1-methyl-2-oxopyrrolidin-3-yl)-5,6-dihydro-4H-benzo[de][1,6]naphthyridine-2- |
| | carboxamide |
| 193 | 8-(Rₐ)-(2-amino-3-cyano-7-fluorobenzo[b]thiophen-4-yl)-7-chloro-9-fluoro-N- |
| | methyl-4-((R)-1-methyl-2-oxopyrrolidin-3-yl)-5,6-dihydro-4H- |
| | benzo[de][1,6]naphthyridine-2-carboxamide |
| 194 | 8-(Rₐ)-(2-amino-3-cyano-7-fluorobenzo[b]thiophen-4-yl)-7-chloro-9-fluoro-N- |
| | methyl-4-((S)-1-methyl-2-oxopyrrolidin-3-yl)-5,6-dihydro-4H- |
| | benzo[de][1,6]naphthyridine-2-carboxamide |
| 195 | 8-(Sₐ)-(2-amino-3-cyano-7-fluorobenzo[b]thiophen-4-yl)-7-chloro-9-fluoro-N- |
| | methyl-4-((R)-1-methyl-2-oxopyrrolidin-3-yl)-5,6-dihydro-4H- |
| | benzo[de][1,6]naphthyridine-2-carboxamide |
| 196 | 8-(Sₐ)-(2-amino-3-cyano-7-fluorobenzo[b]thiophen-4-yl)-7-chloro-9-fluoro-N- |
| | methyl-4-((S)-1-methyl-2-oxopyrrolidin-3-yl)-5,6-dihydro-4H- |
| | benzo[de][1,6]naphthyridine-2-carboxamide |
| 197 | 2-amino-4-(7-chloro-9-fluoro-2-(1-hydroxyethyl)-4-((R)-1-methyl-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 198 | 2-amino-4-(7-chloro-9-fluoro-2-(1-hydroxyethyl)-4-((S)-1-methyl-2-oxopyrrolidin-3- |
| | yl)-5,6-dihydro-4H-benzo[de][1,6]naphthyridin-8-yl)-7-fluorobenzo[b]thiophene-3- |
| | carbonitrile |
| 199 | (Rₐ)-2-amino-4-(7-chloro-9-fluoro-2-((R)-1-hydroxyethyl)-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 200 | (Sₐ)-2-amino-4-(7-chloro-9-fluoro-2-((R)-1-hydroxyethyl)-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 201 | (Rₐ)-2-amino-4-(7-chloro-9-fluoro-2-((S)-1-hydroxyethyl)-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 202 | (Sₐ)-2-amino-4-(7-chloro-9-fluoro-2-((S)-1-hydroxyethyl)-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 203 | (Rₐ)-2-amino-4-(7-chloro-9-fluoro-2-((S)-1-hydroxyethyl)-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 204 | (Sₐ)-2-amino-4-(7-chloro-9-fluoro-2-((S)-1-hydroxyethyl)-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 205 | (Rₐ)-2-amino-4-(7-chloro-9-fluoro-2-((R)-1-hydroxyethyl)-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 206 | (Sₐ)-2-amino-4-(7-chloro-9-fluoro-2-((R)-1-hydroxyethyl)-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 207 | 8-(2-amino-3-cyano-7-fluorobenzo[b]thiophen-4-yl)-7-chloro-9-fluoro-4-((R)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 208 | 8-(2-amino-3-cyano-7-fluorobenzo[b]thiophen-4-yl)-7-chloro-9-fluoro-4-((S)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 209 | 8-(Rₐ)-(2-amino-3-cyano-7-fluorobenzo[b]thiophen-4-yl)-7-chloro-9-fluoro-4-((R)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 210 | 8-(Rₐ)-(2-amino-3-cyano-7-fluorobenzo[b]thiophen-4-yl)-7-chloro-9-fluoro-4-((S)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 211 | 8-(Sₐ)-(2-amino-3-cyano-7-fluorobenzo[b]thiophen-4-yl)-7-chloro-9-fluoro-4-((R)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 212 | 8-(Sₐ)-(2-amino-3-cyano-7-fluorobenzo[b]thiophen-4-yl)-7-chloro-9-fluoro-4-((S)-1- |
| | methyl-2-oxopyrrolidin-3-yl)-N-(1-methylazetidin-3-yl)-5,6-dihydro-4H- |
| | benzo[de][1,6]naphthyridine-2-carboxamide |
| 213 | 8-(2-amino-3-cyano-7-fluorobenzo[b]thiophen-4-yl)-7-chloro-9-fluoro-N-(2-hydroxy- |
| | 2-methylpropyl)-4-((R)-1-methyl-2-oxopyrrolidin-3-yl)-5,6-dihydro-4H- |
| | benzo[de][1,6]naphthyridine-2-carboxamide |
| 214 | 8-(2-amino-3-cyano-7-fluorobenzo[b]thiophen-4-yl)-7-chloro-9-fluoro-N-(2-hydroxy- |
| | 2-methylpropyl)-4-((S)-1-methyl-2-oxopyrrolidin-3-yl)-5,6-dihydro-4H- |
| | benzo[de][1,6]naphthyridine-2-carboxamide |
| 215 | 8-(Rₐ)-(2-amino-3-cyano-7-fluorobenzo[b]thiophen-4-yl)-7-chloro-9-fluoro-N-(2- |
| | hydroxy-2-methylpropyl)-4-((R)-1-methyl-2-oxopyrrolidin-3-yl)-5,6-dihydro-4H- |
| | benzo[de][1,6]naphthyridine-2-carboxamide |
| 216 | 8-(Rₐ)-(2-amino-3-cyano-7-fluorobenzo[b]thiophen-4-yl)-7-chloro-9-fluoro-N-(2- |
| | hydroxy-2-methylpropyl)-4-((S)-1-methyl-2-oxopyrrolidin-3-yl)-5,6-dihydro-4H- |
| | benzo[de][1,6]naphthyridine-2-carboxamide |
| 217 | 8-(Sₐ)-(2-amino-3-cyano-7-fluorobenzo[b]thiophen-4-yl)-7-chloro-9-fluoro-N-(2- |
| | hydroxy-2-methylpropyl)-4-((R)-1-methyl-2-oxopyrrolidin-3-yl)-5,6-dihydro-4H- |
| | benzo[de][1,6]naphthyridine-2-carboxamide |
| 218 | 8-(Sₐ)-(2-amino-3-cyano-7-fluorobenzo[b]thiophen-4-yl)-7-chloro-9-fluoro-N-(2- |
| | hydroxy-2-methylpropyl)-4-((S)-1-methyl-2-oxopyrrolidin-3-yl)-5,6-dihydro-4H- |
| | benzo[de][1,6]naphthyridine-2-carboxamide |
| 219 | N-(8-(2-amino-3-cyano-7-fluorobenzo[b]thiophen-4-yl)-7-chloro-9-fluoro-4-((R)-1- |
| | methyl-2-oxopyrrolidin-3-yl)-5,6-dihydro-4H-benzo[de][1,6]naphthyridin-2- |
| | yl)acetamide |
| 220 | N-(8-(2-amino-3-cyano-7-fluorobenzo[b]thiophen-4-yl)-7-chloro-9-fluoro-4-((S)-1- |
| | methyl-2-oxopyrrolidin-3-yl)-5,6-dihydro-4H-benzo[de][1,6]naphthyridin-2- |
| | yl)acetamide |
| 221 | N-(8-(Rₐ)-(2-amino-3-cyano-7-fluorobenzo[b]thiophen-4-yl)-7-chloro-9-fluoro-4- |
| | ((R)-1-methyl-2-oxopyrrolidin-3-yl)-5,6-dihydro-4H-benzo[de][1,6]naphthyridin-2- |
| | yl)acetamide |
| 222 | N-(8-(Rₐ)-(2-amino-3-cyano-7-fluorobenzo[b]thiophen-4-yl)-7-chloro-9-fluoro-4- |
| | ((S)-1-methyl-2-oxopyrrolidin-3-yl)-5,6-dihydro-4H-benzo[de][1,6]naphthyridin-2- |
| | yl)acetamide |
| 223 | N-(8-(Sₐ)-(2-amino-3-cyano-7-fluorobenzo[b]thiophen-4-yl)-7-chloro-9-fluoro-4- |
| | ((R)-1-methyl-2-oxopyrrolidin-3-yl)-5,6-dihydro-4H-benzo[de][1,6]naphthyridin-2- |
| | yl)acetamide |
| 224 | N-(8-(Sₐ)-(2-amino-3-cyano-7-fluorobenzo[b]thiophen-4-yl)-7-chloro-9-fluoro-4- |
| | ((S)-1-methyl-2-oxopyrrolidin-3-yl)-5,6-dihydro-4H-benzo[de][1,6]naphthyridin-2- |
| | yl)acetamide |
| 225 | 2-amino-4-(7-chloro-2-((difluoromethoxy)methyl)-9-fluoro-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 226 | 2-amino-4-(7-chloro-2-((difluoromethoxy)methyl)-9-fluoro-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 227 | (Rₐ)-2-amino-4-(7-chloro-2-((difluoromethoxy)methyl)-9-fluoro-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 228 | (Rₐ)-2-amino-4-(7-chloro-2-((difluoromethoxy)methyl)-9-fluoro-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 229 | (Sₐ)-2-amino-4-(7-chloro-2-((difluoromethoxy)methyl)-9-fluoro-4-((R)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 230 | (Sₐ)-2-amino-4-(7-chloro-2-((difluoromethoxy)methyl)-9-fluoro-4-((S)-1-methyl-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 231 | 2-amino-4-(7-chloro-9-fluoro-4-((3R,4S)-4-hydroxy-1-(methyl-d₃)-2-oxopyrrolidin-3- |
| | yl)-2-methyl-5,6-dihydro-4H-benzo[de][1,6]naphthyridin-8-yl)-7- |
| | fluorobenzo[b]thiophene-3-carbonitrile |
| 232 | 2-amino-4-(7-chloro-9-fluoro-4-((3S,4R)-4-hydroxy-1-(methyl-d₃)-2-oxopyrrolidin-3- |
| | yl)-2-methyl-5,6-dihydro-4H-benzo[de][1,6]naphthyridin-8-yl)-7- |
| | fluorobenzo[b]thiophene-3-carbonitrile |
| 233 | 2-amino-4-(7-chloro-9-fluoro-4-((3R,4R)-4-hydroxy-1-(methyl-d₃)-2-oxopyrrolidin-3- |
| | yl)-2-methyl-5,6-dihydro-4H-benzo[de][1,6]naphthyridin-8-yl)-7- |
| | fluorobenzo[b]thiophene-3-carbonitrile |
| 234 | 2-amino-4-(7-chloro-9-fluoro-4-((3S,4S)-4-hydroxy-1-(methyl-d₃)-2-oxopyrrolidin-3- |
| | yl)-2-methyl-5,6-dihydro-4H-benzo[de][1,6]naphthyridin-8-yl)-7- |
| | fluorobenzo[b]thiophene-3-carbonitrile |
| 235 | (Rₐ)-2-amino-4-(7-chloro-9-fluoro-4-((3R,4S)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 236 | (Rₐ)-2-amino-4-(7-chloro-9-fluoro-4-((3S,4R)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 237 | (Rₐ)-2-amino-4-(7-chloro-9-fluoro-4-((3R,4R)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 238 | (Rₐ)-2-amino-4-(7-chloro-9-fluoro-4-((3S,4S)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 239 | (Sₐ)-2-amino-4-(7-chloro-9-fluoro-4-((3R,4S)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 240 | (Sₐ)-2-amino-4-(7-chloro-9-fluoro-4-((3S,4R)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 241 | (Sₐ)-2-amino-4-(7-chloro-9-fluoro-4-((3R,4R)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 242 | (Sₐ)-2-amino-4-(7-chloro-9-fluoro-4-((3,4S)-4-hydroxy-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 243 | 2-amino-4-(7-chloro-4-((3R,4S)-4-(difluoromethoxy)-1-(methyl-d₃)-2-oxopyrrolidin- |
| | 3-yl)-9-fluoro-2-methyl-5,6-dihydro-4H-benzo[de][1,6]naphthyridin-8-yl)-7- |
| | fluorobenzo[b]thiophene-3-carbonitrile |
| 244 | 2-amino-4-(7-chloro-4-((3S,4R)-4-(difluoromethoxy)-1-(methyl-d₃)-2-oxopyrrolidin- |
| | 3-yl)-9-fluoro-2-methyl-5,6-dihydro-4H-benzo[de][1,6]naphthyridin-8-yl)-7- |
| | fluorobenzo[b]thiophene-3-carbonitrile |
| 245 | 2-amino-4-(7-chloro-4-((3R,4R)-4-(difluoromethoxy)-1-(methyl-d₃)-2-oxopyrrolidin- |
| | 3-yl)-9-fluoro-2-methyl-5,6-dihydro-4H-benzo[de][1,6]naphthyridin-8-yl)-7- |
| | fluorobenzo[b]thiophene-3-carbonitrile |
| 246 | 2-amino-4-(7-chloro-4-((3S,4S)-4-(difluoromethoxy)-1-(methyl-d₃)-2-oxopyrrolidin- |
| | 3-yl)-9-fluoro-2-methyl-5,6-dihydro-4H-benzo[de][1,6]naphthyridin-8-yl)-7- |
| | fluorobenzo[b]thiophene-3-carbonitrile |
| 247 | (Rₐ)-2-amino-4-(7-chloro-4-((3R,4S)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 248 | (Rₐ)-2-amino-4-(7-chloro-4-((3S,4R)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 249 | (Rₐ)-2-amino-4-(7-chloro-4-((3R,4R)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 250 | (Rₐ)-2-amino-4-(7-chloro-4-((3S,4S)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 251 | (Sₐ)-2-amino-4-(7-chloro-4-((3R,4S)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 252 | (Sₐ)-2-amino-4-(7-chloro-4-((3S,4R)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 253 | (Sₐ)-2-amino-4-(7-chloro-4-((3R,4R)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 254 | (Sₐ)-2-amino-4-(7-chloro-4-((3S,4S)-4-(difluoromethoxy)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 255 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 256 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 257 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 258 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 259 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 260 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 261 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 262 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 263 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 264 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 265 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 266 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 267 | 2-amino-4-(7-chloro-4-((3R,5S)-5-(difluoromethyl)-1-(methyl-d₃)-2-oxopyrrolidin-3- |
| | yl)-9-fluoro-2-methyl-5,6-dihydro-4H-benzo[de][1,6]naphthyridin-8-yl)-7- |
| | fluorobenzo[b]thiophene-3-carbonitrile |
| 268 | 2-amino-4-(7-chloro-4-((3S,5R)-5-(difluoromethyl)-1-(methyl-d₃)-2-oxopyrrolidin-3- |
| | yl)-9-fluoro-2-methyl-5,6-dihydro-4H-benzo[de][1,6]naphthyridin-8-yl)-7- |
| | fluorobenzo[b]thiophene-3-carbonitrile |
| 269 | 2-amino-4-(7-chloro-4-((3R,5R)-5-(difluoromethyl)-1-(methyl-d₃)-2-oxopyrrolidin-3- |
| | yl)-9-fluoro-2-methyl-5,6-dihydro-4H-benzo[de][1,6]naphthyridin-8-yl)-7- |
| | fluorobenzo[b]thiophene-3-carbonitrile |
| 270 | 2-amino-4-(7-chloro-4-((3S,5S)-5-(difluoromethyl)-1-(methyl-d₃)-2-oxopyrrolidin-3- |
| | yl)-9-fluoro-2-methyl-5,6-dihydro-4H-benzo[de][1,6]naphthyridin-8-yl)-7- |
| | fluorobenzo[b]thiophene-3-carbonitrile |
| 271 | (Rₐ)-2-amino-4-(7-chloro-4-((3R,5S)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 272 | (Rₐ)-2-amino-4-(7-chloro-4-((3S,5R)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 273 | (Rₐ)-2-amino-4-(7-chloro-4-((3R,5R)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 274 | (Rₐ)-2-amino-4-(7-chloro-4-((3S,5S)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 275 | (Sₐ)-2-amino-4-(7-chloro-4-((3R,5S)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 276 | (Sₐ)-2-amino-4-(7-chloro-4-((3S,5R)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 277 | (Sₐ)-2-amino-4-(7-chloro-4-((3R,5R)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 278 | (Sₐ)-2-amino-4-(7-chloro-4-((3S,5S)-5-(difluoromethyl)-1-(methyl-d₃)-2- |
| | oxopyrrolidin-3-yl)-9-fluoro-2-methyl-5,6-dihydro-4H-benzo[de][1,6]naphthyridin-8- |
| | yl)-7-fluorobenzo[b]thiophene-3-carbonitrile |
| 279 | 2-amino-4-(7-chloro-9-fluoro-2-methyl-4-((R)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 280 | 2-amino-4-(7-chloro-9-fluoro-2-methyl-4-((S)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 281 | (Rₐ)-2-amino-4-(7-chloro-9-fluoro-2-methyl-4-((R)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 282 | (Rₐ)-2-amino-4-(7-chloro-9-fluoro-2-methyl-4-((S)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 283 | (Sₐ)-2-amino-4-(7-chloro-9-fluoro-2-methyl-4-((R)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 284 | (Sₐ)-2-amino-4-(7-chloro-9-fluoro-2-methyl-4-((S)-4-(methyl-d₃)-5-oxo-4- |
| | azaspiro[2.4]heptan-6-yl)-5,6-dihydro-4H-benzo[de][1,6]naphthyridin-8-yl)-7- |
| | fluorobenzo[b]thiophene-3-carbonitrile |
| 285 | 2-amino-4-(7-chloro-9-fluoro-4-((3R,5S)-5-((methoxy-d3)methyl)-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 286 | 2-amino-4-(7-chloro-9-fluoro-4-((3S,5R)-5-((methoxy-d3)methyl)-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 287 | 2-amino-4-(7-chloro-9-fluoro-4-((3R,5R)-5-((methoxy-d3)methyl)-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 288 | 2-amino-4-(7-chloro-9-fluoro-4-((3S,5S)-5-((methoxy-d3)methyl)-1-(methyl-d₃)-2- |
| | oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 289 | (Rₐ)-2-amino-4-(7-chloro-9-fluoro-4-((3R,5S)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 290 | (Rₐ)-2-amino-4-(7-chloro-9-fluoro-4-((3S,5R)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 291 | (Rₐ)-2-amino-4-(7-chloro-9-fluoro-4-((3R,5R)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 292 | (Rₐ)-2-amino-4-(7-chloro-9-fluoro-4-((3S,5S)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 293 | (Sₐ)-2-amino-4-(7-chloro-9-fluoro-4-((3R,5S)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 294 | (Sₐ)-2-amino-4-(7-chloro-9-fluoro-4-((3S,5R)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 295 | (Sₐ)-2-amino-4-(7-chloro-9-fluoro-4-((3R,5R)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 296 | (Sₐ)-2-amino-4-(7-chloro-9-fluoro-4-((3S,5S)-5-((methoxy-d₃)methyl)-1-(methyl-d₃)- |
| | 2-oxopyrrolidin-3-yl)-2-methyl-5,6-dihydro-4H-benzo[de][1,6]naphthyridin-8-yl)-7- |
| | fluorobenzo[b]thiophene-3-carbonitrile |
| 297 | (2S,4R)-4-(8-(2-amino-3-cyano-7-fluorobenzo[b]thiophen-4-yl)-7-chloro-9-fluoro-2- |
| | methyl-5,6-dihydro-4H-benzo[de][1,6]naphthyridin-4-yl)-N,N-dimethyl-1-(methyl- |
| | d₃)-5-oxopyrrolidine-2-carboxamide |
| 298 | (2R,4S)-4-(8-(2-amino-3-cyano-7-fluorobenzo[b]thiophen-4-yl)-7-chloro-9-fluoro-2- |
| | methyl-5,6-dihydro-4H-benzo[de][1,6]naphthyridin-4-yl)-N,N-dimethyl-1-(methyl- |
| | d₃)-5-oxopyrrolidine-2-carboxamide |
| 299 | (2R,4R)-4-(8-(2-amino-3-cyano-7-fluorobenzo[b]thiophen-4-yl)-7-chloro-9-fluoro-2- |
| | methyl-5,6-dihydro-4H-benzo[de][1,6]naphthyridin-4-yl)-N,N-dimethyl-1-(methyl- |
| | d₃)-5-oxopyrrolidine-2-carboxamide |
| 300 | (2S,4S)-4-(8-(2-amino-3-cyano-7-fluorobenzo[b]thiophen-4-yl)-7-chloro-9-fluoro-2- |
| | methyl-5,6-dihydro-4H-benzo[de][1,6]naphthyridin-4-yl)-N,N-dimethyl-1-(methyl- |
| | d₃)-5-oxopyrrolidine-2-carboxamide |
| 301 | (2S,4R)-4-(8-(Rₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 302 | (2R,4S)-4-(8-(Rₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 303 | (2R,4R)-4-(8-(Rₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 304 | (2S,4S)-4-(8-(Rₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 305 | (2S,4R)-4-(8-(Sₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 306 | (2R,4S)-4-(8-(Sₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 307 | (2R,4R)-4-(8-(Sₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 308 | (2S,4S)-4-(8-(Sₐ)-(2-amino-3-cyano-7-fluorobenzo[b]thiophen-4-yl)-7-chloro-9- |
| | fluoro-2-methyl-5,6-dihydro-4H-benzo[de][1,6]naphthyridin-4-yl)-N,N-dimethyl-1- |
| | (methyl-d₃)-5-oxopyrrolidine-2-carboxamide |
| 309 | 2-amino-4-(7-chloro-9-fluoro-2-methyl-4-((R)-2-methyl-3-oxoisoxazolidin-4-yl)-5,6- |
| | dihydro-4H-benzo[de][1,6]naphthyridin-8-yl)-7-fluorobenzo[b]thiophene-3- |
| | carbonitrile |
| 310 | 2-amino-4-(7-chloro-9-fluoro-2-methyl-4-((S)-2-methyl-3-oxoisoxazolidin-4-yl)-5,6- |
| | dihydro-4H-benzo[de][1,6]naphthyridin-8-yl)-7-fluorobenzo[b]thiophene-3- |
| | carbonitrile |
| 311 | (Rₐ)-2-amino-4-(7-chloro-9-fluoro-2-methyl-4-((R)-2-methyl-3-oxoisoxazolidin-4-yl)- |
| | 5,6-dihydro-4H-benzo[de][1,6]naphthyridin-8-yl)-7-fluorobenzo[b]thiophene-3- |
| | carbonitrile |
| 312 | (Rₐ)-2-amino-4-(7-chloro-9-fluoro-2-methyl-4-((S)-2-methyl-3-oxoisoxazolidin-4-yl)- |
| | 5,6-dihydro-4H-benzo[de][1,6]naphthyridin-8-yl)-7-fluorobenzo[b]thiophene-3- |
| | carbonitrile |
| 313 | (Sₐ)-2-amino-4-(7-chloro-9-fluoro-2-methyl-4-((R)-2-methyl-3-oxoisoxazolidin-4-yl)- |
| | 5,6-dihydro-4H-benzo[de][1,6]naphthyridin-8-yl)-7-fluorobenzo[b]thiophene-3- |
| | carbonitrile |
| 314 | (Sₐ)-2-amino-4-(7-chloro-9-fluoro-2-methyl-4-((S)-2-methyl-3-oxoisoxazolidin-4-yl)- |
| | 5,6-dihydro-4H-benzo[de][1,6]naphthyridin-8-yl)-7-fluorobenzo[b]thiophene-3- |
| | carbonitrile |
| 315 | 2-amino-4-(7-chloro-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 316 | 2-amino-4-(7-chloro-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 317 | 2-amino-4-(7-chloro-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 318 | 2-amino-4-(7-chloro-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 319 | (Rₐ)-2-amino-4-(7-chloro-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 320 | (Rₐ)-2-amino-4-(7-chloro-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 321 | (Rₐ)-2-amino-4-(7-chloro-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 322 | (Rₐ)-2-amino-4-(7-chloro-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 323 | (Sₐ)-2-amino-4-(7-chloro-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 324 | (Sₐ)-2-amino-4-(7-chloro-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 325 | (Sₐ)-2-amino-4-(7-chloro-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 326 | (Sₐ)-2-amino-4-(7-chloro-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 327 | 2-amino-4-(7-chloro-2-(3-(dimethylamino)azetidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 328 | 2-amino-4-(7-chloro-2-(3-(dimethylamino)azetidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 329 | 2-amino-4-(7-chloro-2-(3-(dimethylamino)azetidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 330 | 2-amino-4-(7-chloro-2-(3-(dimethylamino)azetidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 331 | (Rₐ)-2-amino-4-(7-chloro-2-(3-(dimethylamino)azetidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 332 | (Rₐ)-2-amino-4-(7-chloro-2-(3-(dimethylamino)azetidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 333 | (Rₐ)-2-amino-4-(7-chloro-2-(3-(dimethylamino)azetidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 334 | (Rₐ)-2-amino-4-(7-chloro-2-(3-(dimethylamino)azetidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 335 | (Sₐ)-2-amino-4-(7-chloro-2-(3-(dimethylamino)azetidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 336 | (Sₐ)-2-amino-4-(7-chloro-2-(3-(dimethylamino)azetidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 337 | (Sₐ)-2-amino-4-(7-chloro-2-(3-(dimethylamino)azetidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 338 | (Sₐ)-2-amino-4-(7-chloro-2-(3-(dimethylamino)azetidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 339 | 2-amino-4-(7-chloro-2-(3-(dimethylamino)-3-methylazetidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 340 | 2-amino-4-(7-chloro-2-(3-(dimethylamino)-3-methylazetidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 341 | 2-amino-4-(7-chloro-2-(3-(dimethylamino)-3-methylazetidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 342 | 2-amino-4-(7-chloro-2-(3-(dimethylamino)-3-methylazetidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 343 | (Rₐ)-2-amino-4-(7-chloro-2-(3-(dimethylamino)-3-methylazetidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 344 | (Rₐ)-2-amino-4-(7-chloro-2-(3-(dimethylamino)-3-methylazetidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 345 | (Rₐ)-2-amino-4-(7-chloro-2-(3-(dimethylamino)-3-methylazetidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 346 | (Rₐ)-2-amino-4-(7-chloro-2-(3-(dimethylamino)-3-methylazetidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 347 | (Sₐ)-2-amino-4-(7-chloro-2-(3-(dimethylamino)-3-methylazetidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 348 | (Sₐ)-2-amino-4-(7-chloro-2-(3-(dimethylamino)-3-methylazetidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 349 | (Sₐ)-2-amino-4-(7-chloro-2-(3-(dimethylamino)-3-methylazetidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 350 | (Sₐ)-2-amino-4-(7-chloro-2-(3-(dimethylamino)-3-methylazetidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 351 | 2-amino-4-(7-chloro-9-fluoro-4-((3R,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 352 | 2-amino-4-(7-chloro-9-fluoro-4-((3S,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 353 | 2-amino-4-(7-chloro-9-fluoro-4-((3R,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 354 | 2-amino-4-(7-chloro-9-fluoro-4-((3S,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 355 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 356 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 357 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 358 | (Rₐ)-2-amino-4-(7-chloro-9-fluoro-4-((3,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 359 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 360 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 361 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 362 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(3-(S-methylsulfonimidoyl)azetidin-1-yl)-5,6- |
| | dihydro-4H-benzo[de][1,6]naphthyridin-8-yl)-7-fluorobenzo[b]thiophene-3- |
| | carbonitrile |
| 363 | 2-amino-4-(7-chloro-9-fluoro-2-(3-methyl-3-(pyrrolidin-1-yl)azetidin-1-yl)-4- |
| | ((3R,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 364 | 2-amino-4-(7-chloro-9-fluoro-2-(3-methyl-3-(pyrrolidin-1-yl)azetidin-1-yl)-4- |
| | ((3S,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 365 | 2-amino-4-(7-chloro-9-fluoro-2-(3-methyl-3-(pyrrolidin-1-yl)azetidin-1-yl)-4- |
| | ((3R,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 366 | 2-amino-4-(7-chloro-9-fluoro-2-(3-methyl-3-(pyrrolidin-1-yl)azetidin-1-yl)-4- |
| | ((3S,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 367 | (Rₐ)-2-amino-4-(7-chloro-9-fluoro-2-(3-methyl-3-(pyrrolidin-1-yl)azetidin-1-yl)-4- |
| | ((3R,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 368 | (Rₐ)-2-amino-4-(7-chloro-9-fluoro-2-(3-methyl-3-(pyrrolidin-1-yl)azetidin-1-yl)-4- |
| | ((3S,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 369 | (Rₐ)-2-amino-4-(7-chloro-9-fluoro-2-(3-methyl-3-(pyrrolidin-1-yl)azetidin-1-yl)-4- |
| | ((3R,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 370 | (Rₐ)-2-amino-4-(7-chloro-9-fluoro-2-(3-methyl-3-(pyrrolidin-1-yl)azetidin-1-yl)-4- |
| | ((3S,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 371 | (Sₐ)-2-amino-4-(7-chloro-9-fluoro-2-(3-methyl-3-(pyrrolidin-1-yl)azetidin-1-yl)-4- |
| | ((3R,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 372 | (Sₐ)-2-amino-4-(7-chloro-9-fluoro-2-(3-methyl-3-(pyrrolidin-1-yl)azetidin-1-yl)-4- |
| | ((3S,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 373 | (Sₐ)-2-amino-4-(7-chloro-9-fluoro-2-(3-methyl-3-(pyrrolidin-1-yl)azetidin-1-yl)-4- |
| | ((3R,5R)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 374 | (Sₐ)-2-amino-4-(7-chloro-9-fluoro-2-(3-methyl-3-(pyrrolidin-1-yl)azetidin-1-yl)-4- |
| | ((3S,5S)-1-(methyl-d₃)-2-oxo-5-(trifluoromethyl)pyrrolidin-3-yl)-5,6-dihydro-4H- |
| | benzo[de] [1,6]naphthyridin-8-yl)-7-fluorobenzo[b]thiophene-3-carbonitrile |
| 375 | 2-amino-4-(7-chloro-9-fluoro-2-(((2R,7aS)-2-fluorotetrahydro-1H-pyrrolizin-7a(5H)- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 376 | 2-amino-4-(7-chloro-9-fluoro-2-(((2R,7aS)-2-fluorotetrahydro-1H-pyrrolizin-7a(5H)- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 377 | 2-amino-4-(7-chloro-9-fluoro-2-(((2R,7aS)-2-fluorotetrahydro-1H-pyrrolizin-7a(5H)- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 378 | 2-amino-4-(7-chloro-9-fluoro-2-(((2R,7aS)-2-fluorotetrahydro-1H-pyrrolizin-7a(5H)- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 379 | (Rₐ)-2-amino-4-(7-chloro-9-fluoro-2-(((2R,7aS)-2-fluorotetrahydro-1H-pyrrolizin- |
| | 7a(5H)-yl)methoxy)-4-((3R,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 380 | (Rₐ)-2-amino-4-(7-chloro-9-fluoro-2-(((2R,7aS)-2-fluorotetrahydro-1H-pyrrolizin- |
| | 7a(5H)-yl)methoxy)-4-((3S,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 381 | (Rₐ)-2-amino-4-(7-chloro-9-fluoro-2-(((2R,7aS)-2-fluorotetrahydro-1H-pyrrolizin- |
| | 7a(5H)-yl)methoxy)-4-((3R,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 382 | (Rₐ)-2-amino-4-(7-chloro-9-fluoro-2-(((2R,7aS)-2-fluorotetrahydro-1H-pyrrolizin- |
| | 7a(5H)-yl)methoxy)-4-((3S,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 383 | (Sₐ)-2-amino-4-(7-chloro-9-fluoro-2-(((2R,7aS)-2-fluorotetrahydro-1H-pyrrolizin- |
| | 7a(5H)-yl)methoxy)-4-((3R,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 384 | (Sₐ)-2-amino-4-(7-chloro-9-fluoro-2-(((2R,7aS)-2-fluorotetrahydro-1H-pyrrolizin- |
| | 7a(5H)-yl)methoxy)-4-((3S,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 385 | (Sₐ)-2-amino-4-(7-chloro-9-fluoro-2-(((2R,7aS)-2-fluorotetrahydro-1H-pyrrolizin- |
| | 7a(5H)-yl)methoxy)-4-((3R,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 386 | (Sₐ)-2-amino-4-(7-chloro-9-fluoro-2-(((2R,7aS)-2-fluorotetrahydro-1H-pyrrolizin- |
| | 7a(5H)-yl)methoxy)-4-((3S,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 387 | (Rₐ)-2-amino-4-(7-chloro-9-fluoro-2-(((2S,7aR)-2-fluorotetrahydro-1H-pyrrolizin- |
| | 7a(5H)-yl)methoxy)-4-((3R,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 388 | (Sₐ)-2-amino-4-(7-chloro-9-fluoro-2-(((2S,7aR)-2-fluorotetrahydro-1H-pyrrolizin- |
| | 7a(5H)-yl)methoxy)-4-((3R,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 389 | (Rₐ)-2-amino-4-(7-chloro-9-fluoro-2-(((2S,7aR)-2-fluorotetrahydro-1H-pyrrolizin- |
| | 7a(5H)-yl)methoxy)-4-((3S,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 390 | (Sₐ)-2-amino-4-(7-chloro-9-fluoro-2-(((2S,7aR)-2-fluorotetrahydro-1H-pyrrolizin- |
| | 7a(5H)-yl)methoxy)-4-((3S,5R)-1-(methyl-d₃)-2-oxo-5-(trifluoromethyl)pyrrolidin-3- |
| | yl)-5,6-dihydro-4H-benzo[de][1,6]naphthyridine-8-yl)-7-fluorobenzo[b]thiophene-3- |
| | carbonitrile |
| 391 | (Rₐ)-2-amino-4-(7-chloro-9-fluoro-2-(((2S,7aR)-2-fluorotetrahydro-1H-pyrrolizin- |
| | 7a(5H)-yl)methoxy)-4-((3R,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 392 | (Sₐ)-2-amino-4-(7-chloro-9-fluoro-2-(((2S,7aR)-2-fluorotetrahydro-1H-pyrrolizin- |
| | 7a(5H)-yl)methoxy)-4-((3R,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 393 | (Rₐ)-2-amino-4-(7-chloro-9-fluoro-2-(((2S,7aR)-2-fluorotetrahydro-1H-pyrrolizin- |
| | 7a(5H)-yl)methoxy)-4-((3S,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 394 | (Sₐ)-2-amino-4-(7-chloro-9-fluoro-2-(((2S,7aR)-2-fluorotetrahydro-1H-pyrrolizin- |
| | 7a(5H)-yl)methoxy)-4-((3S,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 395 | 2-amino-4-(7-chloro-9-fluoro-4-((3R,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 396 | 2-amino-4-(7-chloro-9-fluoro-4-((3S,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 397 | 2-amino-4-(7-chloro-9-fluoro-4-((3R,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 398 | 2-amino-4-(7-chloro-9-fluoro-4-((3S,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 399 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 400 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 401 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 402 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 403 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 404 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 405 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 406 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-((R)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 407 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 408 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 409 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 410 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 411 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 412 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 413 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 414 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-((S)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 415 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 416 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 417 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 418 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 419 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 420 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 421 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-((S)-1-((S)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 422 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 423 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 424 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 425 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 426 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 427 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 428 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 429 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-((R)-1-((R)-1-methylpyrrolidin-2-yl)ethoxy)-5,6- |
| | dihydro-4H-benzo[de][1,6]naphthyridin-8-yl)-7-fluorobenzo[b]thiophene-3- |
| | carbonitrile |
| 430 | 2-amino-4-(7-chloro-2-(((S)-2-(difluoromethylene)tetrahydro-1H-pyrrolizin-7a(5H)- |
| | yl)methoxy)-9-fluoro-4-((3R,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 431 | 2-amino-4-(7-chloro-2-(((S)-2-(difluoromethylene)tetrahydro-1H-pyrrolizin-7a(5H)- |
| | yl)methoxy)-9-fluoro-4-((3S,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 432 | 2-amino-4-(7-chloro-2-(((S)-2-(difluoromethylene)tetrahydro-1H-pyrrolizin-7a(5H)- |
| | yl)methoxy)-9-fluoro-4-((3R,5R)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 433 | 2-amino-4-(7-chloro-2-(((S)-2-(difluoromethylene)tetrahydro-1H-pyrrolizin-7a(5H)- |
| | yl)methoxy)-9-fluoro-4-((3S,5S)-1-(methyl-d₃)-2-oxo-5-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 434 | (Rₐ)-2-amino-4-(7-chloro-2-(((S)-2-(difluoromethylene)tetrahydro-1H-pyrrolizin- |
| | 7a(5H)-yl)methoxy)-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 435 | (Rₐ)-2-amino-4-(7-chloro-2-(((S)-2-(difluoromethylene)tetrahydro-1H-pyrrolizin- |
| | 7a(5H)-yl)methoxy)-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 436 | (Rₐ)-2-amino-4-(7-chloro-2-(((S)-2-(difluoromethylene)tetrahydro-1H-pyrrolizin- |
| | 7a(5H)-yl)methoxy)-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 437 | (Rₐ)-2-amino-4-(7-chloro-2-(((S)-2-(difluoromethylene)tetrahydro-1H-pyrrolizin- |
| | 7a(5H)-yl)methoxy)-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 438 | (Rₐ)-2-amino-4-(7-chloro-2-(((R)-2-(difluoromethylene)tetrahydro-1H-pyrrolizin- |
| | 7a(5H)-yl)methoxy)-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 439 | (Sₐ)-2-amino-4-(7-chloro-2-(((R)-2-(difluoromethylene)tetrahydro-1H-pyrrolizin- |
| | 7a(5H)-yl)methoxy)-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 440 | (Rₐ)-2-amino-4-(7-chloro-2-(((R)-2-(difluoromethylene)tetrahydro-1H-pyrrolizin- |
| | 7a(5H)-yl)methoxy)-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 441 | (Sₐ)-2-amino-4-(7-chloro-2-(((R)-2-(difluoromethylene)tetrahydro-1H-pyrrolizin- |
| | 7a(5H)-yl)methoxy)-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 442 | (Rₐ)-2-amino-4-(7-chloro-2-(((R)-2-(difluoromethylene)tetrahydro-1H-pyrrolizin- |
| | 7a(5H)-yl)methoxy)-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 443 | (Sₐ)-2-amino-4-(7-chloro-2-(((R)-2-(difluoromethylene)tetrahydro-1H-pyrrolizin- |
| | 7a(5H)-yl)methoxy)-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 444 | (Rₐ)-2-amino-4-(7-chloro-2-(((R)-2-(difluoromethylene)tetrahydro-1H-pyrrolizin- |
| | 7a(5H)-yl)methoxy)-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 445 | (Sₐ)-2-amino-4-(7-chloro-2-(((R)-2-(difluoromethylene)tetrahydro-1H-pyrrolizin- |
| | 7a(5H)-yl)methoxy)-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 446 | (Sₐ)-2-amino-4-(7-chloro-2-(((S)-2-(difluoromethylene)tetrahydro-1H-pyrrolizin- |
| | 7a(5H)-yl)methoxy)-9-fluoro-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)- |
| | 4057-fluorobenzo[b]thiophene-3-carbonitrile |
| 447 | (Sₐ)-2amino-4-(7-chloro-2-(((S)-2-(difluoromethylene)tetrahydro-1H-pyrrolizin- |
| | 7a(5H)-yl)methoxy)-9-fluoro-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 448 | (Sₐ)-2-amino-4-(7-chloro-2-(((S)-2-(difluoromethylene)tetrahydro-1H-pyrrolizin- |
| | 7a(5H)-yl)methoxy)-9-fluoro-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 449 | (Sₐ)-2-amino-4-(7-chloro-2-(((S)-2-(difluoromethylene)tetrahydro-1H-pyrrolizin- |
| | 7a(5H)-yl)methoxy)-9-fluoro-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 450 | 2-amino-4-(7-chloro-9-fluoro-4-((3R,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 451 | 2-amino-4-(7-chloro-9-fluoro-4-((3S,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 452 | 2-amino-4-(7-chloro-9-fluoro-4-((3R,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 453 | 2-amino-4-(7-chloro-9-fluoro-4-((3S,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 454 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 455 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 4564 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 457 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 458 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 459 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 460 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 461 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(((S)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 462 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 463 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 464 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 465 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 466 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 467 | Sₐ)-2-amino-4-(7-chloro-9-fluoro-4-((3R,5R)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 468 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 469 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(((R)-1-methylpyrrolidin-2-yl)methoxy)-5,6- |
| | dihydro-4H-benzo[de][1,6]naphthyridin-8-yl)-7-fluorobenzo[b]thiophene-3- |
| | carbonitrile |
| 470 | 2-amino-4-(7-chloro-9-fluoro-2-(((2S,4R)-4-fluoro-1-methylpyrrolidin-2-yl)methoxy)- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 471 | 2-amino-4-(7-chloro-9-fluoro-2-(((2R,4S)-4-fluoro-1-methylpyrrolidin-2-yl)methoxy)- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 472 | 2-amino-4-(7-chloro-9-fluoro-2-(((2R,4R)-4-fluoro-1-methylpyrrolidin-2-yl)methoxy)- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 473 | 2-amino-4-(7-chloro-9-fluoro-2-(((2S,4S)-4-fluoro-1-methylpyrrolidin-2-yl)methoxy)- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 474 | (Rₐ)-2-amino-4-(7-chloro-9-fluoro-2-(((2S,4R)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 475 | (Rₐ)-2-amino-4-(7-chloro-9-fluoro-2-(((2R,4S)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 476 | (Rₐ)-2-amino-4-(7-chloro-9-fluoro-2-(((2R,4R)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 477 | (Rₐ)-2-amino-4-(7-chloro-9-fluoro-2-(((2S,4S)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 478 | (Sₐ)-2-amino-4-(7-chloro-9-fluoro-2-(((2S,4R)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 479 | (Rₐ)-2-amino-4-(7-chloro-9-fluoro-2-(((2S,4R)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 480 | (Sₐ)-2-amino-4-(7-chloro-9-fluoro-2-(((2S,4R)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 481 | (Rₐ)-2-amino-4-(7-chloro-9-fluoro-2-(((2S,4R)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 482 | (Sₐ)-2-amino-4-(7-chloro-9-fluoro-2-(((2S,4R)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 483 | (Rₐ)-2-amino-4-(7-chloro-9-fluoro-2-(((2S,4R)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 484 | (Sₐ)-2-amino-4-(7-chloro-9-fluoro-2-(((2S,4R)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 485 | (Rₐ)-2-amino-4-(7-chloro-9-fluoro-2-(((2R,4R)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 486 | (Sₐ)-2-amino-4-(7-chloro-9-fluoro-2-(((2R,4R)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 487 | (Rₐ)-2-amino-4-(7-chloro-9-fluoro-2-(((2R,4R)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 488 | (Sₐ)-2-amino-4-(7-chloro-9-fluoro-2-(((2R,4R)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 489 | (Rₐ)-2-amino-4-(7-chloro-9-fluoro-2-(((2R,4R)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 490 | (Sₐ)-2-amino-4-(7-chloro-9-fluoro-2-(((2R,4R)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 491 | (Rₐ)-2-amino-4-(7-chloro-9-fluoro-2-(((2S,4S)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 492 | (Sₐ)-2-amino-4-(7-chloro-9-fluoro-2-(((2S,4S)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 493 | (Rₐ)-2-amino-4-(7-chloro-9-fluoro-2-(((2S,4S)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 494 | (Sₐ)-2-amino-4-(7-chloro-9-fluoro-2-(((2S,4S)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 495 | (Rₐ)-2-amino-4-(7-chloro-9-fluoro-2-(((2S,4S)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 496 | (Sₐ)-2-amino-4-(7-chloro-9-fluoro-2-(((2S,4S)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 497 | (Rₐ)-2-amino-4-(7-chloro-9-fluoro-2-(((2R,4S)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 498 | (Sₐ)-2-amino-4-(7-chloro-9-fluoro-2-(((2R,4S)-4-fluoro-1-methylpyrrolidin-2- |
| | yl)methoxy)-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 499 | (Rₐ)-2-amino-4-(7-chloro-9-fluoro-2-(((2R,4S)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 500 | (Sₐ)-2-amino-4-(7-chloro-9-fluoro-2-(((2R,4S)-4-fluoro-1-methylpyrrolidin-2- |
| | yl)methoxy)-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 501 | (Rₐ)-2-amino-4-(7-chloro-9-fluoro-2-(((2R,4S)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 502 | (Sₐ)-2-amino-4-(7-chloro-9-fluoro-2-(((2R,4S)-4-fluoro-1-methylpyrrolidin-2- |
| | yl)methoxy)-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 503 | (Sₐ)-2-amino-4-(7-chloro-9-fluoro-2-(((2R,4S)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 504 | (Sₐ)-2-amino-4-(7-chloro-9-fluoro-2-(((2R,4R)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 505 | (Sₐ)-2-amino-4-(7-chloro-9-fluoro-2-(((2S,4S)-4-fluoro-1-methylpyrrolidin-2- |
| | yl)methoxy)-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 506 | 2-amino-4-(7-chloro-9-fluoro-4-((3R,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 507 | 2-amino-4-(7-chloro-9-fluoro-4-((3S,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 508 | 2-amino-4-(7-chloro-9-fluoro-4-((3R,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 509 | 2-amino-4-(7-chloro-9-fluoro-4-((3S,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 510 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 511 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 512 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 513 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 514 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 515 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 516 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 517 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(1H-1,2,4-triazol-1-yl)-5,6-dihydro-4H- |
| | benzo[de][1,6]naphthyridin-8-yl)-7-fluorobenzo[b]thiophene-3-carbonitrile |
| 518 | 8-(2-amino-3-cyano-7-fluorobenzo[b]thiophen-4-yl)-7-chloro-9-fluoro-4-((3R,5S)-1- |
| | (methyl-d₃)-2-oxo-5-(trifluoromethyl)pyrrolidin-3-yl)-N-(tetrahydro-2H-pyran-4-yl)- |
| | 5,6-dihydro-4H-benzo[de][1,6]naphthyridine-2-carboxamide |
| 519 | 8-(2-amino-3-cyano-7-fluorobenzo[b]thiophen-4-yl)-7-chloro-9-fluoro-4-((3S,5R)-1- |
| | (methyl-d₃)-2-oxo-5-(trifluoromethyl)pyrrolidin-3-yl)-N-(tetrahydro-2H-pyran-4-yl)- |
| | 5,6-dihydro-4H-benzo[de][1,6]naphthyridine-2-carboxamide |
| 520 | 8-(2-amino-3-cyano-7-fluorobenzo[b]thiophen-4-yl)-7-chloro-9-fluoro-4-((3R,5R)-1- |
| | (methyl-d₃)-2-oxo-5-(trifluoromethyl)pyrrolidin-3-yl)-N-(tetrahydro-2H-pyran-4-yl)- |
| | 5,6-dihydro-4H-benzo[de][1,6]naphthyridine-2-carboxamide |
| 521 | 8-(2-amino-3-cyano-7-fluorobenzo[b]thiophen-4-yl)-7-chloro-9-fluoro-4-((3S,5S)-1- |
| | (methyl-d₃)-2-oxo-5-(trifluoromethyl)pyrrolidin-3-yl)-N-(tetrahydro-2H-pyran-4-yl)- |
| | 5,6-dihydro-4H-benzo[de][1,6]naphthyridine-2-carboxamide |
| 522 | 8-(Rₐ)-(2-amino-3-cyano-7-fluorobenzo[b]thiophen-4-yl)-7-chloro-9-fluoro-4- |
| | ((3R,5S)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 523 | 8-(Rₐ)-(2-amino-3-cyano-7-fluorobenzo[b]thiophen-4-yl)-7-chloro-9-fluoro-4- |
| | ((3S,5R)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 524 | 8-(Rₐ)-(2-amino-3-cyano-7-fluorobenzo[b]thiophen-4-yl)-7-chloro-9-fluoro-4- |
| | ((3R,5R)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 525 | 8-(Rₐ)-(2-amino-3-cyano-7-fluorobenzo[b]thiophen-4-yl)-7-chloro-9-fluoro-4- |
| | ((3S,5S)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 526 | 8-(Sₐ)-(2-amino-3-cyano-7-fluorobenzo[b]thiophen-4-yl)-7-chloro-9-fluoro-4- |
| | ((3R,5S)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 527 | 8-(Sₐ)-(2-amino-3-cyano-7-fluorobenzo[b]thiophen-4-yl)-7-chloro-9-fluoro-4- |
| | ((3S,5R)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 528 | 8-(Sₐ)-(2-amino-3-cyano-7-fluorobenzo[b]thiophen-4-yl)-7-chloro-9-fluoro-4- |
| | ((3R,5R)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 529 | 8-(Sₐ)-(2-amino-3-cyano-7-fluorobenzo[b]thiophen-4-yl)-7-chloro-9-fluoro-4- |
| | ((3S,5S)-1-(methyl-d₃)-2-oxo-5-(trifluoromethyl)pyrrolidin-3-yl)-N-(tetrahydro-2H- |
| | pyran-4-yl)-5,6-dihydro-4H-benzo[de][1,6]naphthyridine-2-carboxamide |
| 530 | 8-(2-amino-3-cyano-7-fluorobenzo[b]thiophen-4-yl)-7-chloro-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridine-2-carbonitrile |
| 531 | 8-(2-amino-3-cyano-7-fluorobenzo[b]thiophen-4-yl)-7-chloro-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridine-2-carbonitrile |
| 532 | 8-(2-amino-3-cyano-7-fluorobenzo[b]thiophen-4-yl)-7-chloro-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridine-2-carbonitrile |
| 533 | 8-(2-amino-3-cyano-7-fluorobenzo[b]thiophen-4-yl)-7-chloro-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridine-2-carbonitrile |
| 534 | 8-(Rₐ)-(2-amino-3-cyano-7-fluorobenzo[b]thiophen-4-yl)-7-chloro-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 535 | 8-(Rₐ)-(2-amino-3-cyano-7-fluorobenzo[b]thiophen-4-yl)-7-chloro-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 536 | 8-(Rₐ)-(2-amino-3-cyano-7-fluorobenzo[b]thiophen-4-yl)-7-chloro-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 537 | 8-(Rₐ)-(2-amino-3-cyano-7-fluorobenzo[b]thiophen-4-yl)-7-chloro-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 538 | 8-(Sₐ)-(2-amino-3-cyano-7-fluorobenzo[b]thiophen-4-yl)-7-chloro-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 539 | 8-(Sₐ)-(2-amino-3-cyano-7-fluorobenzo[b]thiophen-4-yl)-7-chloro-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 540 | 8-(Sₐ)-(2-amino-3-cyano-7-fluorobenzo[b]thiophen-4-yl)-7-chloro-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 541 | 8-(Sₐ)-(2-amino-3-cyano-7-fluorobenzo[b]thiophen-4-yl)-7-chloro-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-2-carbonitrile |
| 542 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 543 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 544 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 545 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 546 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 547 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 548 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 549 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 550 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 551 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 552 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 553 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 554 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 555 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 556 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 557 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 558 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 559 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 560 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 561 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 562 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 563 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 564 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 565 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-5- |
| | fluorobenzo[b]thiophene-3-carbonitrile |
| 566 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 567 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 568 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 569 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 570 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 571 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 572 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 573 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 574 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 575 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 576 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 577 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-6,7- |
| | difluorobenzo[b]thiophene-3-carbonitrile |
| 578 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 579 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 580 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 581 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 582 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 583 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 584 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 585 | (Rₐ)-2-amino-4-(7-chloro-9-fluoro-2-methyl-4-((3,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 586 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 587 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 588 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 589 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-5,7- |
| | difluorobenzo[b]thiophene-3-carbonitrile |
| 590 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 591 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 592 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 593 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 594 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 595 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 596 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 597 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 598 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 599 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 600 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 601 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorothieno[3,2-c]pyridine-3-carbonitrile |
| 602 | 2-amino-7-fluoro-4-(9-fluoro-2-methyl-4-((3R,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 603 | 2-amino-7-fluoro-4-(9-fluoro-2-methyl-4-((3S,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 604 | 2-amino-7-fluoro-4-(9-fluoro-2-methyl-4-((3R,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 605 | 2-amino-7-fluoro-4-(9-fluoro-2-methyl-4-((3S,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 606 | (Rₐ)-2-amino-7-fluoro-4-(9-fluoro-2-methyl-4-((3R,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 607 | (Rₐ)-2-amino-7-fluoro-4-(9-fluoro-2-methyl-4-((3S,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 608 | (Rₐ)-2-amino-7-fluoro-4-(9-fluoro-2-methyl-4-((3R,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 609 | (Rₐ)-2-amino-7-fluoro-4-(9-fluoro-2-methyl-4-((3S,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 610 | (Sₐ)-2-amino-7-fluoro-4-(9-fluoro-2-methyl-4-((3R,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 611 | (Sₐ)-2-amino-7-fluoro-4-(9-fluoro-2-methyl-4-((3S,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 612 | (Sₐ)-2-amino-7-fluoro-4-(9-fluoro-2-methyl-4-((3R,5R)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 613 | (Sₐ)-2-amino-7-fluoro-4-(9-fluoro-2-methyl-4-((3S,5S)-1-(methyl-d₃)-2-oxo-5- |
| | (trifluoromethyl)pyrrolidin-3-yl)-7-(trifluoromethyl)-5,6-dihydro-4H-benzo[de] |
| | [1,6]naphthyridin-8-yl)benzo[b]thiophene-3-carbonitrile |
| 614 | 2-amino-4-(7-(difluoromethyl)-9-fluoro-2-methyl-4-((3R,5S)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 615 | 2-amino-4-(7-(difluoromethyl)-9-fluoro-2-methyl-4-((3S,5R)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 616 | 2-amino-4-(7-(difluoromethyl)-9-fluoro-2-methyl-4-((3R,5R)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 617 | 2-amino-4-(7-(difluoromethyl)-9-fluoro-2-methyl-4-((3S,5S)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 618 | (Rₐ)-2-amino-4-(7-(difluoromethyl)-9-fluoro-2-methyl-4-((3R,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 619 | (Rₐ)-2-amino-4-(7-(difluoromethyl)-9-fluoro-2-methyl-4-((3S,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 620 | (Rₐ)-2-amino-4-(7-(difluoromethyl)-9-fluoro-2-methyl-4-((3R,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 621 | (Rₐ)-2-amino-4-(7-(difluoromethyl)-9-fluoro-2-methyl-4-((3S,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 622 | (Sₐ)-2-amino-4-(7-(difluoromethyl)-9-fluoro-2-methyl-4-((3R,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 623 | (Sₐ)-2-amino-4-(7-(difluoromethyl)-9-fluoro-2-methyl-4-((3S,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 624 | (Sₐ)-2-amino-4-(7-(difluoromethyl)-9-fluoro-2-methyl-4-((3R,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 625 | (Sₐ)-2-amino-4-(7-(difluoromethyl)-9-fluoro-2-methyl-4-((3S,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 626 | 2-amino-4-(7-chloro-3,9-difluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 627 | 2-amino-4-(7-chloro-3,9-difluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 628 | 2-amino-4-(7-chloro-3,9-difluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 629 | 2-amino-4-(7-chloro-3,9-difluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 630 | (Rₐ)-2-amino-4-(7-chloro-3,9-difluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 631 | (Rₐ)-2-amino-4-(7-chloro-3,9-difluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 632 | (Rₐ)-2-amino-4-(7-chloro-3,9-difluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 633 | (Rₐ)-2-amino-4-(7-chloro-3,9-difluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 634 | (Sₐ)-2-amino-4-(7-chloro-3,9-difluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 635 | (Sₐ)-2-amino-4-(7-chloro-3,9-difluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 636 | (Sₐ)-2-amino-4-(7-chloro-3,9-difluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 637 | (Sₐ)-2-amino-4-(7-chloro-3,9-difluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 638 | 2-amino-7-fluoro-4-(9-fluoro-2,7-dimethyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 639 | 2-amino-7-fluoro-4-(9-fluoro-2,7-dimethyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 640 | 2-amino-7-fluoro-4-(9-fluoro-2,7-dimethyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 641 | 2-amino-7-fluoro-4-(9-fluoro-2,7-dimethyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 642 | (Rₐ)-2-amino-7-fluoro-4-(9-fluoro-2,7-dimethyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 643 | (Rₐ)-2-amino-7-fluoro-4-(9-fluoro-2,7-dimethyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 644 | (Rₐ)-2-amino-7-fluoro-4-(9-fluoro-2,7-dimethyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 645 | (Rₐ)-2-amino-7-fluoro-4-(9-fluoro-2,7-dimethyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 646 | (Sₐ)-2-amino-7-fluoro-4-(9-fluoro-2,7-dimethyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 647 | (Sₐ)-2-amino-7-fluoro-4-(9-fluoro-2,7-dimethyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 648 | (Sₐ)-2-amino-7-fluoro-4-(9-fluoro-2,7-dimethyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 649 | (Sₐ)-2-amino-7-fluoro-4-(9-fluoro-2,7-dimethyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)benzo[b]thiophene-3-carbonitrile |
| 650 | 2-amino-4-(2-(difluoromethyl)-9-fluoro-7-methyl-4-((3R,5S)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 651 | 2-amino-4-(2-(difluoromethyl)-9-fluoro-7-methyl-4-((3S,5R)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 652 | 2-amino-4-(2-(difluoromethyl)-9-fluoro-7-methyl-4-((3R,5R)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 653 | 2-amino-4-(2-(difluoromethyl)-9-fluoro-7-methyl-4-((3S,5S)-1-(methyl-d₃)-2-oxo-5- |
| | (trifluoromethyl)pyrrolidin-3-yl)-5,6-dihydro-4H-benzo[de] [1,6]naphthyridin-8-yl)-7- |
| | fluorobenzo[b]thiophene-3-carbonitrile |
| 654 | (Rₐ)-2-amino-4-(2-(difluoromethyl)-9-fluoro-7-methyl-4-((3R,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 655 | (Rₐ)-2-amino-4-(2-(difluoromethyl)-9-fluoro-7-methyl-4-((3S,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 656 | (Rₐ)-2-amino-4-(2-(difluoromethyl)-9-fluoro-7-methyl-4-((3R,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 657 | (Rₐ)-2-amino-4-(2-(difluoromethyl)-9-fluoro-7-methyl-4-((3S,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 658 | (Sₐ)-2-amino-4-(2-(difluoromethyl)-9-fluoro-7-methyl-4-((3R,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 659 | (Sₐ)-2-amino-4-(2-(difluoromethyl)-9-fluoro-7-methyl-4-((3S,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 660 | (Sₐ)-2-amino-4-(2-(difluoromethyl)-9-fluoro-7-methyl-4-((3R,5R)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 661 | (Sₐ)-2-amino-4-(2-(difluoromethyl)-9-fluoro-7-methyl-4-((3S,5S)-1-(methyl-d₃)-2- |
| | oxo-5-(trifluoromethyl)pyrrolidin-3-yl)-5,6-dihydro-4H-benzo[de] [1,6]naphthyridin-8- |
| | yl)-7-fluorobenzo[b]thiophene-3-carbonitrile |
| 662 | 2-amino-4-(2-(3-(dimethylamino)-3-methylazetidin-1-yl)-9-fluoro-7-methyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 663 | 2-amino-4-(2-(3-(dimethylamino)-3-methylazetidin-1-yl)-9-fluoro-7-methyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 664 | 2-amino-4-(2-(3-(dimethylamino)-3-methylazetidin-1-yl)-9-fluoro-7-methyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 665 | 2-amino-4-(2-(3-(dimethylamino)-3-methylazetidin-1-yl)-9-fluoro-7-methyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 666 | (Rₐ)-2-amino-4-(2-(3-(dimethylamino)-3-methylazetidin-1-yl)-9-fluoro-7-methyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 667 | (Rₐ)-2-amino-4-(2-(3-(dimethylamino)-3-methylazetidin-1-yl)-9-fluoro-7-methyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 668 | (Rₐ)-2-amino-4-(2-(3-(dimethylamino)-3-methylazetidin-1-yl)-9-fluoro-7-methyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 669 | (Rₐ)-2-amino-4-(2-(3-(dimethylamino)-3-methylazetidin-1-yl)-9-fluoro-7-methyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 670 | (Sₐ)-2-amino-4-(2-(3-(dimethylamino)-3-methylazetidin-1-yl)-9-fluoro-7-methyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 671 | (Sₐ)-2-amino-4-(2-(3-(dimethylamino)-3-methylazetidin-1-yl)-9-fluoro-7-methyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 672 | (Sₐ)-2-amino-4-(2-(3-(dimethylamino)-3-methylazetidin-1-yl)-9-fluoro-7-methyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 673 | (Sₐ)-2-amino-4-(2-(3-(dimethylamino)-3-methylazetidin-1-yl)-9-fluoro-7-methyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 674 | 2-amino-4-(2-(3-(dimethylamino)azetidin-1-yl)-9-fluoro-7-methyl-4-((3R,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 675 | 2-amino-4-(2-(3-(dimethylamino)azetidin-1-yl)-9-fluoro-7-methyl-4-((3S,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 676 | 2-amino-4-(2-(3-(dimethylamino)azetidin-1-yl)-9-fluoro-7-methyl-4-((3R,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 677 | 2-amino-4-(2-(3-(dimethylamino)azetidin-1-yl)-9-fluoro-7-methyl-4-((3S,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 678 | (Rₐ)-2-amino-4-(2-(3-(dimethylamino)azetidin-1-yl)-9-fluoro-7-methyl-4-((3R,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 679 | (Rₐ)-2-amino-4-(2-(3-(dimethylamino)azetidin-1-yl)-9-fluoro-7-methyl-4-((3S,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 680 | (Rₐ)-2-amino-4-(2-(3-(dimethylamino)azetidin-1-yl)-9-fluoro-7-methyl-4-((3R,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 681 | (Rₐ)-2-amino-4-(2-(3-(dimethylamino)azetidin-1-yl)-9-fluoro-7-methyl-4-((3S,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 682 | (Sₐ)-2-amino-4-(2-(3-(dimethylamino)azetidin-1-yl)-9-fluoro-7-methyl-4-((3R,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 683 | (Sₐ)-2-amino-4-(2-(3-(dimethylamino)azetidin-1-yl)-9-fluoro-7-methyl-4-((3S,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 684 | (Sₐ)-2-amino-4-(2-(3-(dimethylamino)azetidin-1-yl)-9-fluoro-7-methyl-4-((3R,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 685 | (Sₐ)-2-amino-4-(2-(3-(dimethylamino)azetidin-1-yl)-9-fluoro-7-methyl-4-((3S,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 686 | 2-amino-4-(7-chloro-9-fluoro-4-((3S,5R)-5-fluoro-1-methyl-2-oxopiperidin-3-yl)-2- |
| | methyl-5,6-dihydro-4H-benzo[de][1,6]naphthyridin-8-yl)-7-fluorobenzo[b]thiophene- |
| | 3-carbonitrile |
| 687 | 2-amino-4-(7-chloro-9-fluoro-4-((3R,5S)-5-fluoro-1-methyl-2-oxopiperidin-3-yl)-2- |
| | methyl-5,6-dihydro-4H-benzo[de][1,6]naphthyridin-8-yl)-7-fluorobenzo[b]thiophene- |
| | 3-carbonitrile |
| 688 | 2-amino-4-(7-chloro-9-fluoro-4-((3R,5R)-5-fluoro-1-methyl-2-oxopiperidin-3-yl)-2- |
| | methyl-5,6-dihydro-4H-benzo[de][1,6]naphthyridin-8-yl)-7-fluorobenzo[b]thiophene- |
| | 3-carbonitrile |
| 689 | 2-amino-4-(7-chloro-9-fluoro-4-((3S,5S)-5-fluoro-1-methyl-2-oxopiperidin-3-yl)-2- |
| | methyl-5,6-dihydro-4H-benzo[de][1,6]naphthyridin-8-yl)-7-fluorobenzo[b]thiophene- |
| | 3-carbonitrile |
| 690 | (Rₐ)-2-amino-4-(7-chloro-9-fluoro-4-((3S,5R)-5-fluoro-1-methyl-2-oxopiperidin-3- |
| | yl)-2-methyl-5,6-dihydro-4H-benzo[de][1,6]naphthyridin-8-yl)-7- |
| | fluorobenzo[b]thiophene-3-carbonitrile |
| 691 | (Rₐ)-2-amino-4-(7-chloro-9-fluoro-4-((3R,5S)-5-fluoro-1-methyl-2-oxopiperidin-3- |
| | yl)-2-methyl-5,6-dihydro-4H-benzo[de][1,6]naphthyridin-8-yl)-7- |
| | fluorobenzo[b]thiophene-3-carbonitrile |
| 692 | (Rₐ)-2-amino-4-(7-chloro-9-fluoro-4-((3R,5R)-5-fluoro-1-methyl-2-oxopiperidin-3- |
| | yl)-2-methyl-5,6-dihydro-4H-benzo[de][1,6]naphthyridin-8-yl)-7- |
| | fluorobenzo[b]thiophene-3-carbonitrile |
| 693 | (Rₐ)-2-amino-4-(7-chloro-9-fluoro-4-((3S,5S)-5-fluoro-1-methyl-2-oxopiperidin-3-yl)- |
| | 2-methyl-5,6-dihydro-4H-benzo[de][1,6]naphthyridin-8-yl)-7- |
| | fluorobenzo[b]thiophene-3-carbonitrile |
| 694 | (Sₐ)-2-amino-4-(7-chloro-9-fluoro-4-((3S,5R)-5-fluoro-1-methyl-2-oxopiperidin-3-yl)- |
| | 2-methyl-5,6-dihydro-4H-benzo[de][1,6]naphthyridin-8-yl)-7- |
| | fluorobenzo[b]thiophene-3-carbonitrile |
| 695 | (Sₐ)-2-amino-4-(7-chloro-9-fluoro-4-((3R,5S)-5-fluoro-1-methyl-2-oxopiperidin-3-yl)- |
| | 2-methyl-5,6-dihydro-4H-benzo[de][1,6]naphthyridin-8-yl)-7- |
| | fluorobenzo[b]thiophene-3-carbonitrile |
| 696 | (Sₐ)-2-amino-4-(7-chloro-9-fluoro-4-((3R,5R)-5-fluoro-1-methyl-2-oxopiperidin-3- |
| | yl)-2-methyl-5,6-dihydro-4H-benzo[de][1,6]naphthyridin-8-yl)-7- |
| | fluorobenzo[b]thiophene-3-carbonitrile |
| 697 | (Sₐ)-2-amino-4-(7-chloro-9-fluoro-4-((3S,5S)-5-fluoro-1-methyl-2-oxopiperidin-3-yl)- |
| | 2-methyl-5,6-dihydro-4H-benzo[de][1,6]naphthyridin-8-yl)-7- |
| | fluorobenzo[b]thiophene-3-carbonitrile |
| 698 | 2-amino-4-(7-chloro-9-fluoro-2-(3-hydroxy-3-methylbut-1-yn-1-yl)-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 699 | 2-amino-4-(7-chloro-9-fluoro-2-(3-hydroxy-3-methylbut-1-yn-1-yl)-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 700 | 2-amino-4-(7-chloro-9-fluoro-2-(3-hydroxy-3-methylbut-1-yn-1-yl)-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 701 | 2-amino-4-(7-chloro-9-fluoro-2-(3-hydroxy-3-methylbut-1-yn-1-yl)-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 702 | (Rₐ)-2-amino-4-(7-chloro-9-fluoro-2-(3-hydroxy-3-methylbut-1-yn-1-yl)-4-((3R,5S)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 703 | (Rₐ)-2-amino-4-(7-chloro-9-fluoro-2-(3-hydroxy-3-methylbut-1-yn-1-yl)-4-((3S,5R)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 704 | (Rₐ)-2-amino-4-(7-chloro-9-fluoro-2-(3-hydroxy-3-methylbut-1-yn-1-yl)-4-((3R,5R)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 705 | (Rₐ)-2-amino-4-(7-chloro-9-fluoro-2-(3-hydroxy-3-methylbut-1-yn-1-yl)-4-((3S,5S)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 706 | (Sₐ)-2-amino-4-(7-chloro-9-fluoro-2-(3-hydroxy-3-methylbut-1-yn-1-yl)-4-((3R,5S)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 707 | (Sₐ)-2-amino-4-(7-chloro-9-fluoro-2-(3-hydroxy-3-methylbut-1-yn-1-yl)-4-((3S,5R)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 708 | (Sₐ)-2-amino-4-(7-chloro-9-fluoro-2-(3-hydroxy-3-methylbut-1-yn-1-yl)-4-((3R,5R)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 709 | (Sₐ)-2-amino-4-(7-chloro-9-fluoro-2-(3-hydroxy-3-methylbut-1-yn-1-yl)-4-((3S,5S)- |
| | 1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 710 | 2-amino-4-(7-chloro-2-(3-(dimethylamino)prop-1-yn-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 711 | 2-amino-4-(7-chloro-2-(3-(dimethylamino)prop-1-yn-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 712 | 2-amino-4-(7-chloro-2-(3-(dimethylamino)prop-1-yn-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 713 | 2-amino-4-(7-chloro-2-(3-(dimethylamino)prop-1-yn-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 714 | (Rₐ)-2-amino-4-(7-chloro-2-(3-(dimethylamino)prop-1-yn-1-yl)-9-fluoro-4-((3R,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 715 | (Rₐ)-2-amino-4-(7-chloro-2-(3-(dimethylamino)prop-1-yn-1-yl)-9-fluoro-4-((3S,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 716 | (Rₐ)-2-amino-4-(7-chloro-2-(3-(dimethylamino)prop-1-yn-1-yl)-9-fluoro-4-((3R,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 717 | (Rₐ)-2-amino-4-(7-chloro-2-(3-(dimethylamino)prop-1-yn-1-yl)-9-fluoro-4-((3S,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 718 | (Sₐ)-2-amino-4-(7-chloro-2-(3-(dimethylamino)prop-1-yn-1-yl)-9-fluoro-4-((3R,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 719 | (Sₐ)-2-amino-4-(7-chloro-2-(3-(dimethylamino)prop-1-yn-1-yl)-9-fluoro-4-((3S,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 720 | (Sₐ)-2-amino-4-(7-chloro-2-(3-(dimethylamino)prop-1-yn-1-yl)-9-fluoro-4-((3R,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 721 | (Sₐ)-2-amino-4-(7-chloro-2-(3-(dimethylamino)prop-1-yn-1-yl)-9-fluoro-4-((3S,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 722 | 2-amino-4-(7-chloro-2-(3-(dimethylamino)prop-1-yn-1-yl)-9-fluoro-4-((3R,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 723 | 2-amino-4-(7-chloro-2-(3-(dimethylamino)prop-1-yn-1-yl)-9-fluoro-4-((3S,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 724 | 2-amino-4-(7-chloro-2-(3-(dimethylamino)prop-1-yn-1-yl)-9-fluoro-4-((3R,5R)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 725 | 2-amino-4-(7-chloro-2-(3-(dimethylamino)prop-1-yn-1-yl)-9-fluoro-4-((3,5S)-1- |
| | (methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 726 | (Rₐ)-2-amino-4-(7-chloro-2-(3-(dimethylamino)prop-1-yn-1-yl)-9-fluoro-4-((3R,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 727 | (Rₐ)-2-amino-4-(7-chloro-2-(3-(dimethylamino)prop-1-yn-1-yl)-9-fluoro-4-((3S,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 728 | (Sₐ)-2-amino-4-(7-chloro-2-(3-(dimethylamino)prop-1-yn-1-yl)-9-fluoro-4-((3R,5S)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 729 | (Sₐ)-2-amino-4-(7-chloro-2-(3-(dimethylamino)prop-1-yn-1-yl)-9-fluoro-4-((3S,5R)- |
| | 1-(methyl-d₃)-2-oxo-5-(trifluoromethyl)pyrrolidin-3-yl)-5,6-dihydro-4H-benzo[de] |
| | [1,6]naphthyridin-8-yl)-7-fluorobenzo[b]thiophene-3-carbonitrile |
| 730 | 2-amino-4-(7-chloro-9-fluoro-2-methyl-4-((6S,8aR)-5-oxooctahydroindolizin-6-yl)- |
| | 5,6-dihydro-4H-benzo[de][1,6]naphthyridin-8-yl)-7-fluorobenzo[b]thiophene-3- |
| | carbonitrile |
| 731 | 2-amino-4-(7-chloro-9-fluoro-2-methyl-4-((6S,8aS)-5-oxooctahydroindolizin-6-yl)- |
| | 5,6-dihydro-4H-benzo[de][1,6]naphthyridin-8-yl)-7-fluorobenzo[b]thiophene-3- |
| | carbonitrile |
| 732 | 2-amino-4-(7-chloro-9-fluoro-2-methyl-4-((6R,8aR)-5-oxooctahydroindolizin-6-yl)- |
| | 5,6-dihydro-4H-benzo[de][1,6]naphthyridin-8-yl)-7-fluorobenzo[b]thiophene-3- |
| | carbonitrile |
| 733 | 2-amino-4-(7-chloro-9-fluoro-2-methyl-4-((6R,8aS)-5-oxooctahydroindolizin-6-yl)- |
| | 5,6-dihydro-4H-benzo[de][1,6]naphthyridin-8-yl)-7-fluorobenzo[b]thiophene-3- |
| | carbonitrile |
| 734 | (Rₐ)-2-amino-4-(7-chloro-9-fluoro-2-methyl-4-((6S,8aR)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 735 | (Sₐ)-2-amino-4-(7-chloro-9-fluoro-2-methyl-4-((6S,8aR)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 736 | (Rₐ)-2-amino-4-(7-chloro-9-fluoro-2-methyl-4-((6S,8aS)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 737 | (Sₐ)-2-amino-4-(7-chloro-9-fluoro-2-methyl-4-((6S,8aS)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 738 | (Rₐ)-2-amino-4-(7-chloro-9-fluoro-2-methyl-4-((6R,8aR)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 739 | (Sₐ)-2-amino-4-(7-chloro-9-fluoro-2-methyl-4-((6R,8aR)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 740 | (Rₐ)-2-amino-4-(7-chloro-9-fluoro-2-methyl-4-((6R,8aS)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 741 | (Sₐ)-2-amino-4-(7-chloro-9-fluoro-2-methyl-4-((6R,8aS)-5-oxooctahydroindolizin-6- |
| | yl)-5,6-dihydro-4H-benzo[de][1,6]naphthyridin-8-yl)-7-fluorobenzo[b]thiophene-3- |
| | carbonitrile |
| 742 | 2-amino-4-(7-chloro-9-fluoro-4-((3R,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 743 | 2-amino-4-(7-chloro-9-fluoro-4-((3R,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 744 | 2-amino-4-(7-chloro-9-fluoro-4-((3S,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 745 | 2-amino-4-(7-chloro-9-fluoro-4-((3S,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 746 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 747 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 748 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 749 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 750 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 751 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 752 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 753 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-(1-methyl-1,6-diazaspiro[3.3]heptan-6-yl)-5,6- |
| | dihydro-4H-benzo[de][1,6]naphthyridin-8-yl)-7-fluorobenzo[b]thiophene-3- |
| | carbonitrile |
| 754 | 2-amino-4-(7-chloro-2-(3-(dimethylamino)-4-fluoropyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 755 | 2-amino-4-(7-chloro-2-(3-(dimethylamino)-4-fluoropyrrolidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 756 | 2-amino-4-(7-chloro-2-(3-(dimethylamino)-4-fluoropyrrolidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 757 | 2-amino-4-(7-chloro-2-(3-(dimethylamino)-4-fluoropyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 758 | (Rₐ)-2-amino-4-(7-chloro-2-((3R,4S)-3-(dimethylamino)-4-fluoropyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 759 | (Sₐ)-2-amino-4-(7-chloro-2-((3R,4S)-3-(dimethylamino)-4-fluoropyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 760 | (Rₐ)-2-amino-4-(7-chloro-2-((3S,4R)-3-(dimethylamino)-4-fluoropyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 761 | (Sₐ)-2-amino-4-(7-chloro-2-((3S,4R)-3-(dimethylamino)-4-fluoropyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 762 | (Rₐ)-2-amino-4-(7-chloro-2-((3R,4S)-3-(dimethylamino)-4-fluoropyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 763 | (Sₐ)-2-amino-4-(7-chloro-2-((3R,4S)-3-(dimethylamino)-4-fluoropyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 764 | (Rₐ)-2-amino-4-(7-chloro-2-((3S,4R)-3-(dimethylamino)-4-fluoropyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 765 | (Sₐ)-2-amino-4-(7-chloro-2-((3S,4R)-3-(dimethylamino)-4-fluoropyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 766 | 2-amino-4-(7-chloro-9-fluoro-4-((3R,5S)-1-(methyl-d₃)-2-oxo-5- |
| | (trifluoromethyl)pyrrolidin-3-yl)-2-(5-methyl-2,5-diazabicyclo[2.2.1]heptan-2-yl)-5,6- |
| | dihydro-4H-benzo[de][1,6]naphthyridin-8-yl)-7-fluorobenzo[b]thiophene-3- |
| | carbonitrile |
| 767 | 2-amino-4-(7-chloro-9-fluoro-4-((3R,5R)-1-(methyl-d₃)-2-oxo-5- |
| | (trifluoromethyl)pyrrolidin-3-yl)-2-(5-methyl-2,5-diazabicyclo[2.2.1]heptan-2-yl)-5,6- |
| | dihydro-4H-benzo[de][1,6]naphthyridin-8-yl)-7-fluorobenzo[b]thiophene-3- |
| | carbonitrile |
| 768 | 2-amino-4-(7-chloro-9-fluoro-4-((3S,5R)-1-(methyl-d₃)-2-oxo-5- |
| | (trifluoromethyl)pyrrolidin-3-yl)-2-(5-methyl-2,5-diazabicyclo[2.2.1]heptan-2-yl)-5,6- |
| | dihydro-4H-benzo[de][1,6]naphthyridin-8-yl)-7-fluorobenzo[b]thiophene-3- |
| | carbonitrile |
| 769 | 2-amino-4-(7-chloro-9-fluoro-4-((3S,5S)-1-(methyl-d₃)-2-oxo-5- |
| | (trifluoromethyl)pyrrolidin-3-yl)-2-(5-methyl-2,5-diazabicyclo[2.2.1]heptan-2-yl)-5,6- |
| | dihydro-4H-benzo[de][1,6]naphthyridin-8-yl)-7-fluorobenzo[b]thiophene-3- |
| | carbonitrile |
| 770 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((1S,4S)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 771 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((1S,4S)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 772 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((1R,4R)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 773 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((1R,4R)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 774 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((1S,4S)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 775 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((1S,4S)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 776 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((1R,4R)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 777 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((1R,4R)-5-methyl-2,5-diazabicyclo[2.2.1]heptan- |
| | 2-yl)-5,6-dihydro-4H-benzo[de][1,6]naphthyridin-8-yl)-7-fluorobenzo[b]thiophene-3- |
| | carbonitrile |
| 778 | 2-amino-4-(7-chloro-9-fluoro-2-methyl-4-((3R,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 779 | 2-amino-4-(7-chloro-9-fluoro-2-methyl-4-((3R,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 780 | 2-amino-4-(7-chloro-9-fluoro-2-methyl-4-((3S,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 781 | 2-amino-4-(7-chloro-9-fluoro-2-methyl-4-((3S,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 782 | (Rₐ)-2-amino-4-(7-chloro-9-fluoro-2-methyl-4-((3R,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 783 | (Sₐ)-2-amino-4-(7-chloro-9-fluoro-2-methyl-4-((3R,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 784 | (Rₐ)-2-amino-4-(7-chloro-9-fluoro-2-methyl-4-((3S,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 785 | (Sₐ)-2-amino-4-(7-chloro-9-fluoro-2-methyl-4-((3S,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 786 | (Rₐ)-2-amino-4-(7-chloro-9-fluoro-2-methyl-4-((3R,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 787 | (Sₐ)-2-amino-4-(7-chloro-9-fluoro-2-methyl-4-((3R,5R)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 788 | (Rₐ)-2-amino-4-(7-chloro-9-fluoro-2-methyl-4-((3S,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 789 | (Sₐ)-2-amino-4-(7-chloro-9-fluoro-2-methyl-4-((3S,5S)-1-(methyl-d₃)-2-oxo-5- |
| | phenylpyrrolidin-3-yl)-5,6-dihydro-4H-benzo[de][1,6]naphthyridin-8-yl)-7- |
| | fluorobenzo[b]thiophene-3-carbonitrile |
| 790 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 791 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 792 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 793 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 794 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 795 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 796 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 797 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 798 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 799 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 800 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 801 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-b]pyridine-3-carbonitrile |
| 802 | 2-amino-4-(7-chloro-2-((dimethyl(oxo)-λ⁶-sulfaneylidene)amino)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 803 | 2-amino-4-(7-chloro-2-((dimethyl(oxo)-λ⁶-sulfaneylidene)amino)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 804 | 2-amino-4-(7-chloro-2-((dimethyl(oxo)-λ⁶-sulfaneylidene)amino)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 805 | 2-amino-4-(7-chloro-2-((dimethyl(oxo)-λ⁶-sulfaneylidene)amino)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 806 | (Rₐ)-2-amino-4-(7-chloro-2-((dimethyl(oxo)-λ⁶-sulfaneylidene)amino)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile; and |
| 807 | (Sₐ)-2-amino-4-(7-chloro-2-((dimethyl(oxo)-λ⁶-sulfaneylidene)amino)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 808 | (Rₐ)-2-amino-4-(7-chloro-2-((dimethyl(oxo)-λ⁶-sulfaneylidene)amino)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 809 | (Sₐ)-2-amino-4-(7-chloro-2-((dimethyl(oxo)-λ⁶-sulfaneylidene)amino)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 810 | (Rₐ)-2-amino-4-(7-chloro-2-((dimethyl(oxo)-λ⁶-sulfaneylidene)amino)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile; and |
| 811 | (Sₐ)-2-amino-4-(7-chloro-2-((dimethyl(oxo)-λ⁶-sulfaneylidene)amino)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 812 | (Rₐ)-2-amino-4-(7-chloro-2-((dimethyl(oxo)-λ⁶-sulfaneylidene)amino)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile; and |
| 813 | (Sₐ)-2-amino-4-(7-chloro-2-((dimethyl(oxo)-λ⁶-sulfaneylidene)amino)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 814 | 2-amino-4-(7-chloro-2-((S)-3-(dimethylamino)pyrrolidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 815 | 2-amino-4-(7-chloro-2-((R)-3-(dimethylamino)pyrrolidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 816 | 2-amino-4-(7-chloro-2-((S)-3-(dimethylamino)pyrrolidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 817 | 2-amino-4-(7-chloro-2-((R)-3-(dimethylamino)pyrrolidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 818 | 2-amino-4-(7-chloro-2-((S)-3-(dimethylamino)pyrrolidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 819 | 2-amino-4-(7-chloro-2-((R)-3-(dimethylamino)pyrrolidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 820 | 2-amino-4-(7-chloro-2-((S)-3-(dimethylamino)pyrrolidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 821 | 2-amino-4-(7-chloro-2-((R)-3-(dimethylamino)pyrrolidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 822 | (Rₐ)-2-amino-4-(7-chloro-2-((S)-3-(dimethylamino)pyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 823 | (Sₐ)-2-amino-4-(7-chloro-2-((S)-3-(dimethylamino)pyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 824 | (Rₐ)-2-amino-4-(7-chloro-2-((R)-3-(dimethylamino)pyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 825 | (Sₐ)-2-amino-4-(7-chloro-2-((R)-3-(dimethylamino)pyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 826 | (Rₐ)-2-amino-4-(7-chloro-2-((S)-3-(dimethylamino)pyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 827 | (Sₐ)-2-amino-4-(7-chloro-2-((S)-3-(dimethylamino)pyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 828 | (Rₐ)-2-amino-4-(7-chloro-2-((R)-3-(dimethylamino)pyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 829 | (Sₐ)-2-amino-4-(7-chloro-2-((R)-3-(dimethylamino)pyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 830 | 2-amino-4-(7-chloro-2-(3-(dimethylamino)-3-methylpyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 831 | 2-amino-4-(7-chloro-2-(3-(dimethylamino)-3-methylpyrrolidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 832 | 2-amino-4-(7-chloro-2-(3-(dimethylamino)-3-methylpyrrolidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 833 | 2-amino-4-(7-chloro-2-(3-(dimethylamino)-3-methylpyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 834 | (Rₐ)-2-amino-4-(7-chloro-2-((R)-3-(dimethylamino)-3-methylpyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 835 | (Sₐ)-2-amino-4-(7-chloro-2-((R)-3-(dimethylamino)-3-methylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 836 | (Rₐ)-2-amino-4-(7-chloro-2-((S)-3-(dimethylamino)-3-methylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 837 | (Sₐ)-2-amino-4-(7-chloro-2-((S)-3-(dimethylamino)-3-methylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 838 | (Rₐ)-2-amino-4-(7-chloro-2-((R)-3-(dimethylamino)-3-methylpyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 839 | (Sₐ)-2-amino-4-(7-chloro-2-((R)-3-(dimethylamino)-3-methylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 840 | (Rₐ)-2-amino-4-(7-chloro-2-((S)-3-(dimethylamino)-3-methylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 841 | (Sₐ)-2-amino-4-(7-chloro-2-((S)-3-(dimethylamino)-3-methylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 842 | 2-amino-4-(7-chloro-9-fluoro-4-((3R,5S)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 843 | 2-amino-4-(7-chloro-9-fluoro-4-((3S,5R)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 844 | 2-amino-4-(7-chloro-9-fluoro-4-((3R,5S)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 845 | 2-amino-4-(7-chloro-9-fluoro-4-((3R,5S)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 846 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 847 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 848 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 849 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 850 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 851 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((3aS,6aS)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 852 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 853 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((3aR,6aR)-1-methylhexahydropyrrolo[3,4-b]pyrrol- |
| | 5(1H)-yl)-5,6-dihydro-4H-benzo[de][1,6]naphthyridin-8-yl)-7- |
| | fluorobenzo[b]thiophene-3-carbonitrile |
| 854 | 2-amino-4-(7-chloro-2-(3-cyclopropyl-3-hydroxyazetidin-1-yl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 855 | 2-amino-4-(7-chloro-2-(3-cyclopropyl-3-hydroxyazetidin-1-yl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 856 | 2-amino-4-(7-chloro-2-(3-cyclopropyl-3-hydroxyazetidin-1-yl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 857 | 2-amino-4-(7-chloro-2-(3-cyclopropyl-3-hydroxyazetidin-1-yl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 858 | (Rₐ)-2-amino-4-(7-chloro-2-(3-cyclopropyl-3-hydroxyazetidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 859 | (Sₐ)-2-amino-4-(7-chloro-2-(3-cyclopropyl-3-hydroxyazetidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 860 | (Rₐ)-2-amino-4-(7-chloro-2-(3-cyclopropyl-3-hydroxyazetidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 861 | (Sₐ)-2-amino-4-(7-chloro-2-(3-cyclopropyl-3-hydroxyazetidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 862 | (Rₐ)-2-amino-4-(7-chloro-2-(3-cyclopropyl-3-hydroxyazetidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 863 | (Sₐ)-2-amino-4-(7-chloro-2-(3-cyclopropyl-3-hydroxyazetidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 864 | (Rₐ)-2-amino-4-(7-chloro-2-(3-cyclopropyl-3-hydroxyazetidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 865 | (Sₐ)-2-amino-4-(7-chloro-2-(3-cyclopropyl-3-hydroxyazetidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 867 | 2-amino-4-(7-chloro-2-(3-(dimethylamino)-3-ethylpyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 868 | 2-amino-4-(7-chloro-2-(3-(dimethylamino)-3-ethylpyrrolidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 869 | 2-amino-4-(7-chloro-2-(3-(dimethylamino)-3-ethylpyrrolidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 870 | 2-amino-4-(7-chloro-2-(3-(dimethylamino)-3-ethylpyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 871 | (Rₐ)-2-amino-4-(7-chloro-2-((R)-3-(dimethylamino)-3-ethylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 872 | (Sₐ)-2-amino-4-(7-chloro-2-((R)-3-(dimethylamino)-3-ethylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 873 | (Rₐ)-2-amino-4-(7-chloro-2-((S)-3-(dimethylamino)-3-ethylpyrrolidin-1-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 874 | (Sₐ)-2-amino-4-(7-chloro-2-((S)-3-(dimethylamino)-3-ethylpyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 875 | (Rₐ)-2-amino-4-(7-chloro-2-((R)-3-(dimethylamino)-3-ethylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 876 | (Sₐ)-2-amino-4-(7-chloro-2-((R)-3-(dimethylamino)-3-ethylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 878 | (Rₐ)-2-amino-4-(7-chloro-2-((S)-3-(dimethylamino)-3-ethylpyrrolidin-1-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 879 | (Sₐ)-2-amino-4-(7-chloro-2-((S)-3-(dimethylamino)-3-ethylpyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 880 | 2-amino-4-(7-chloro-2-(3-cyclopropyl-3-(dimethylamino)pyrrolidin-1-yl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 881 | 2-amino-4-(7-chloro-2-(3-cyclopropyl-3-(dimethylamino)pyrrolidin-1-yl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 882 | 2-amino-4-(7-chloro-2-(3-cyclopropyl-3-(dimethylamino)pyrrolidin-1-yl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 883 | 2-amino-4-(7-chloro-2-(3-cyclopropyl-3-(dimethylamino)pyrrolidin-1-yl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 884 | (Rₐ)-2-amino-4-(7-chloro-2-((S)-3-cyclopropyl-3-(dimethylamino)pyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 885 | (Sₐ)-2-amino-4-(7-chloro-2-((S)-3-cyclopropyl-3-(dimethylamino)pyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 886 | (Rₐ)-2-amino-4-(7-chloro-2-((R)-3-cyclopropyl-3-(dimethylamino)pyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 887 | (Sₐ)-2-amino-4-(7-chloro-2-((R)-3-cyclopropyl-3-(dimethylamino)pyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 888 | (Rₐ)-2-amino-4-(7-chloro-2-((S)-3-cyclopropyl-3-(dimethylamino)pyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 889 | (Sₐ)-2-amino-4-(7-chloro-2-((S)-3-cyclopropyl-3-(dimethylamino)pyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 890 | (Rₐ)-2-amino-4-(7-chloro-2-((R)-3-cyclopropyl-3-(dimethylamino)pyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 891 | (Sₐ)-2-amino-4-(7-chloro-2-((R)-3-cyclopropyl-3-(dimethylamino)pyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 892 | 2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 893 | 2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 894 | 2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 895 | 2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 896 | (Rₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 897 | (Sₐ)-2-amino-4-(7-chloro-9-fluoro-2-methyl-4-((3R,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 898 | (Rₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 899 | (Sₐ)-2-amino-4-(7-chloro-9-fluoro-2-methyl-4-((3S,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 900 | (Rₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 901 | (Sₐ)-2-amino-4-(7-chloro-9-fluoro-2-methyl-4-((3R,5R)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 902 | (Rₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 903 | (Sₐ)-2-amino-4-(7-chloro-9-fluoro-2-methyl-4-((3S,5S)-1-(methyl-d₃)-2-oxo-5- |
| | (trifluoromethyl)pyrrolidin-3-yl)-5,6-dihydro-4H-benzo[de][1,6]naphthyridin-8- |
| | yl)thieno[2,3-c]pyridine-3-carbonitrile |
| 904 | 8-(2-amino-3-cyano-7-fluorobenzo[b]thiophen-4-yl)-7-chloro-9-fluoro-2-methyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 905 | 8-(2-amino-3-cyano-7-fluorobenzo[b]thiophen-4-yl)-7-chloro-9-fluoro-2-methyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 906 | 8-(2-amino-3-cyano-7-fluorobenzo[b]thiophen-4-yl)-7-chloro-9-fluoro-2-methyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 907 | 8-(2-amino-3-cyano-7-fluorobenzo[b]thiophen-4-yl)-7-chloro-9-fluoro-2-methyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 908 | (Rₐ)-8-(2-amino-3-cyano-7-fluorobenzo[b]thiophen-4-yl)-7-chloro-9-fluoro-2-methyl- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 909 | (Sₐ)-8-(2-amino-3-cyano-7-fluorobenzo[b]thiophen-4-yl)-7-chloro-9-fluoro-2-methyl- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 910 | (Rₐ)-8-(2-amino-3-cyano-7-fluorobenzo[b]thiophen-4-yl)-7-chloro-9-fluoro-2-methyl- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 911 | (Sₐ)-8-(2-amino-3-cyano-7-fluorobenzo[b]thiophen-4-yl)-7-chloro-9-fluoro-2-methyl- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 912 | (Rₐ)-8-(2-amino-3-cyano-7-fluorobenzo[b]thiophen-4-yl)-7-chloro-9-fluoro-2-methyl- |
| | 4-((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 913 | (Sₐ)-8-(2-amino-3-cyano-7-fluorobenzo[b]thiophen-4-yl)-7-chloro-9-fluoro-2-methyl- |
| | 4-((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 914 | (Rₐ)-8-(2-amino-3-cyano-7-fluorobenzo[b]thiophen-4-yl)-7-chloro-9-fluoro-2-methyl- |
| | 4-((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 915 | (Sₐ)-8-(2-amino-3-cyano-7-fluorobenzo[b]thiophen-4-yl)-7-chloro-9-fluoro-2-methyl- |
| | 4-((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 916 | 8-(2-amino-3-cyano-7-fluorobenzo[b]thiophen-4-yl)-9-fluoro-2,7-dimethyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 917 | 8-(2-amino-3-cyano-7-fluorobenzo[b]thiophen-4-yl)-9-fluoro-2,7-dimethyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 918 | 8-(2-amino-3-cyano-7-fluorobenzo[b]thiophen-4-yl)-9-fluoro-2,7-dimethyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 919 | 8-(2-amino-3-cyano-7-fluorobenzo[b]thiophen-4-yl)-9-fluoro-2,7-dimethyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 920 | (Rₐ)-8-(2-amino-3-cyano-7-fluorobenzo[b]thiophen-4-yl)-9-fluoro-2,7-dimethyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 921 | (Sₐ)-8-(2-amino-3-cyano-7-fluorobenzo[b]thiophen-4-yl)-9-fluoro-2,7-dimethyl-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 922 | (Rₐ)-8-(2-amino-3-cyano-7-fluorobenzo[b]thiophen-4-yl)-9-fluoro-2,7-dimethyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 923 | (Sₐ)-8-(2-amino-3-cyano-7-fluorobenzo[b]thiophen-4-yl)-9-fluoro-2,7-dimethyl-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 924 | (Rₐ)-8-(2-amino-3-cyano-7-fluorobenzo[b]thiophen-4-yl)-9-fluoro-2,7-dimethyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 925 | (Sₐ)-8-(2-amino-3-cyano-7-fluorobenzo[b]thiophen-4-yl)-9-fluoro-2,7-dimethyl-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 926 | (Rₐ)-8-(2-amino-3-cyano-7-fluorobenzo[b]thiophen-4-yl)-9-fluoro-2,7-dimethyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 927 | (Sₐ)-8-(2-amino-3-cyano-7-fluorobenzo[b]thiophen-4-yl)-9-fluoro-2,7-dimethyl-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridine-3-carbonitrile |
| 928 | 2-amino-4-(2-(azetidin-1-ylmethyl)-7-chloro-9-fluoro-4-((3R,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 929 | 2-amino-4-(2-(azetidin-1-ylmethyl)-7-chloro-9-fluoro-4-((3R,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 930 | 2-amino-4-(2-(azetidin-1-ylmethyl)-7-chloro-9-fluoro-4-((3S,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 931 | 2-amino-4-(2-(azetidin-1-ylmethyl)-7-chloro-9-fluoro-4-((3S,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 932 | (Rₐ)-2-amino-4-(2-(azetidin-1-ylmethyl)-7-chloro-9-fluoro-4-((3R,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 933 | (Sₐ)-2-amino-4-(2-(azetidin-1-ylmethyl)-7-chloro-9-fluoro-4-((3R,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 934 | (Rₐ)-2-amino-4-(2-(azetidin-1-ylmethyl)-7-chloro-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 935 | (Sₐ)-2-amino-4-(2-(azetidin-1-ylmethyl)-7-chloro-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 936 | (Rₐ)-2-amino-4-(2-(azetidin-1-ylmethyl)-7-chloro-9-fluoro-4-((3R,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 937 | (Sₐ)-2-amino-4-(2-(azetidin-1-ylmethyl)-7-chloro-9-fluoro-4-((3R,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 938 | (Rₐ)-2-amino-4-(2-(azetidin-1-ylmethyl)-7-chloro-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 939 | (Sₐ)-2-amino-4-(2-(azetidin-1-ylmethyl)-7-chloro-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 940 | 2-amino-4-(7-chloro-9-fluoro-4-((3R,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 941 | 2-amino-4-(7-chloro-9-fluoro-4-((3R,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 942 | 2-amino-4-(7-chloro-9-fluoro-4-((3S,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 943 | 2-amino-4-(7-chloro-9-fluoro-4-((3S,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 944 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 945 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 946 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 947 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 948 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 949 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 950 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 951 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-(trifluoromethyl)-5,6-dihydro-4H- |
| | benzo[de][1,6]naphthyridin-8-yl)-7-fluorobenzo[b]thiophene-3-carbonitrile |
| 952 | 2-amino-4-(7-chloro-9-fluoro-2-methyl-4-((3R,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 953 | 2-amino-4-(7-chloro-9-fluoro-2-methyl-4-((3R,5S)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 954 | 2-amino-4-(7-chloro-9-fluoro-2-methyl-4-((3S,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 955 | 2-amino-4-(7-chloro-9-fluoro-2-methyl-4-((3S,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 956 | (Rₐ)-2-amino-4-(7-chloro-9-fluoro-2-methyl-4-((3R,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 957 | (Sₐ)-2-amino-4-(7-chloro-9-fluoro-2-methyl-4-((3R,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 958 | (Rₐ)-2-amino-4-(7-chloro-9-fluoro-2-methyl-4-((3S,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 959 | (Sₐ)-2-amino-4-(7-chloro-9-fluoro-2-methyl-4-((3S,5R)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 960 | (Rₐ)-2-amino-4-(7-chloro-9-fluoro-2-methyl-4-((3R,5S)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 961 | (Sₐ)-2-amino-4-(7-chloro-9-fluoro-2-methyl-4-((3R,5S)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 962 | (Rₐ)-2-amino-4-(7-chloro-9-fluoro-2-methyl-4-((3S,5S)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 963 | (Sₐ)-2-amino-4-(7-chloro-9-fluoro-2-methyl-4-((3S,5S)-1-methyl-2-oxo-5- |
| | (trifluoromethyl)piperidin-3-yl)-5,6-dihydro-4H-benzo[de][1,6]naphthyridin-8-yl)-7- |
| | fluorobenzo[b]thiophene-3-carbonitrile |
| 964 | 2-amino-4-(7-chloro-2-((3S,4R)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 965 | 2-amino-4-(7-chloro-2-((3R,4R)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 966 | 2-amino-4-(7-chloro-2-((3S,4S)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 967 | 2-amino-4-(7-chloro-2-((3R,4R)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 968 | (Rₐ)-2-amino-4-(7-chloro-2-((3S,4R)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 969 | (Sₐ)-2-amino-4-(7-chloro-2-((3S,4R)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 970 | (Rₐ)-2-amino-4-(7-chloro-2-((3R,4S)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 971 | (Sₐ)-2-amino-4-(7-chloro-2-((3R,4S)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 972 | (Rₐ)-2-amino-4-(7-chloro-2-((3S,4R)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 973 | (Sₐ)-2-amino-4-(7-chloro-2-((3S,4R)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 974 | (Rₐ)-2-amino-4-(7-chloro-2-((3R,4S)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 975 | (Sₐ)-2-amino-4-(7-chloro-2-((3R,4S)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3- |
| | carbonitrile |
| 976 | 2-amino-4-(7-chloro-2-(((1,1-dioxidothietan-3-yl)oxy)methyl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 977 | 2-amino-4-(7-chloro-2-(((1,1-dioxidothietan-3-yl)oxy)methyl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 978 | 2-amino-4-(7-chloro-2-(((1,1-dioxidothietan-3-yl)oxy)methyl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 979 | 2-amino-4-(7-chloro-2-(((1,1-dioxidothietan-3-yl)oxy)methyl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 980 | (Rₐ)-2-amino-4-(7-chloro-2-(((1,1-dioxidothietan-3-yl)oxy)methyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 981 | (Sₐ)-2-amino-4-(7-chloro-2-(((1,1-dioxidothietan-3-yl)oxy)methyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 982 | (Rₐ)-2-amino-4-(7-chloro-2-(((1,1-dioxidothietan-3-yl)oxy)methyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 983 | (Sₐ)-2-amino-4-(7-chloro-2-(((1,1-dioxidothietan-3-yl)oxy)methyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 984 | (Rₐ)-2-amino-4-(7-chloro-2-(((1,1-dioxidothietan-3-yl)oxy)methyl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 985 | (Sₐ)-2-amino-4-(7-chloro-2-(((1,1-dioxidothietan-3-yl)oxy)methyl)-9-fluoro-4- |
| | ((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 986 | (Rₐ)-2-amino-4-(7-chloro-2-(((1,1-dioxidothietan-3-yl)oxy)methyl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 987 | (Sₐ)-2-amino-4-(7-chloro-2-(((1,1-dioxidothietan-3-yl)oxy)methyl)-9-fluoro-4- |
| | ((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 988 | 2-amino-4-(7-chloro-9-fluoro-4-((3R,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 989 | 2-amino-4-(7-chloro-9-fluoro-4-((3R,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 990 | 2-amino-4-(7-chloro-9-fluoro-4-((3S,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 991 | 2-amino-4-(7-chloro-9-fluoro-4-((3S,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 992 | (Rₐ)-2-amino-4-(7-chloro-9-fluoro-4-((3R,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 993 | (Sₐ)-2-amino-4-(7-chloro-9-fluoro-4-((3R,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 994 | (Rₐ)-2-amino-4-(7-chloro-9-fluoro-4-((3S,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 995 | (Sₐ)-2-amino-4-(7-chloro-9-fluoro-4-((3S,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 996 | (Rₐ)-2-amino-4-(7-chloro-9-fluoro-4-((3R,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 997 | (Sₐ)-2-amino-4-(7-chloro-9-fluoro-4-((3R,5R)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 998 | (Rₐ)-2-amino-4-(7-chloro-9-fluoro-4-((3S,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 999 | (Sₐ)-2-amino-4-(7-chloro-9-fluoro-4-((3S,5S)-1-(methyl-d₃)-2-oxo-5- |
| | (trifluoromethyl)pyrrolidin-3-yl)-2-((2-(methylsulfonyl)ethoxy)methyl)-5,6-dihydro- |
| | 4H-benzo[de][1,6]naphthyridin-8-yl)-7-fluorobenzo[b]thiophene-3-carbonitrile |
| 1000 | 2-amino-4-(7-chloro-9-fluoro-2-((methoxy-d₃)methyl)-4-((3R,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1001 | 2-amino-4-(7-chloro-9-fluoro-2-((methoxy-d₃)methyl)-4-((3R,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1002 | 2-amino-4-(7-chloro-9-fluoro-2-((methoxy-d₃)methyl)-4-((3S,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1003 | 2-amino-4-(7-chloro-9-fluoro-2-((methoxy-d₃)methyl)-4-((3S,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1004 | (Rₐ)-2-amino-4-(7-chloro-9-fluoro-2-((methoxy-d₃)methyl)-4-((3R,5S)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1005 | (Sₐ)-2-amino-4-(7-chloro-9-fluoro-2-((methoxy-d₃)methyl)-4-((3R,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1006 | (Rₐ)-2-amino-4-(7-chloro-9-fluoro-2-((methoxy-d₃)methyl)-4-((3S,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1007 | (Sₐ)-2-amino-4-(7-chloro-9-fluoro-2-((methoxy-d₃)methyl)-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1008 | (Rₐ)-2-amino-4-(7-chloro-9-fluoro-2-((methoxy-d₃)methyl)-4-((3R,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1009 | (Sₐ)-2-amino-4-(7-chloro-9-fluoro-2-((methoxy-d₃)methyl)-4-((3R,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1010 | (Rₐ)-2-amino-4-(7-chloro-9-fluoro-2-((methoxy-d₃)methyl)-4-((3S,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1011 | (Sₐ)-2-amino-4-(7-chloro-9-fluoro-2-((methoxy-d₃)methyl)-4-((3S,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1012 | 2-amino-4-(7-chloro-2-((difluoromethoxy)methyl)-9-fluoro-4-((3R,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1013 | 2-amino-4-(7-chloro-2-((difluoromethoxy)methyl)-9-fluoro-4-((3R,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1014 | 2-amino-4-(7-chloro-2-((difluoromethoxy)methyl)-9-fluoro-4-((3S,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1015 | 2-amino-4-(7-chloro-2-((difluoromethoxy)methyl)-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1016 | (Rₐ)-2-amino-4-(7-chloro-2-((difluoromethoxy)methyl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1017 | (Sₐ)-2-amino-4-(7-chloro-2-((difluoromethoxy)methyl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1018 | (Rₐ)-2-amino-4-(7-chloro-2-((difluoromethoxy)methyl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1019 | (Sₐ)-2-amino-4-(7-chloro-2-((difluoromethoxy)methyl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1020 | (Rₐ)-2-amino-4-(7-chloro-2-((difluoromethoxy)methyl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1021 | (Sₐ)-2-amino-4-(7-chloro-2-((difluoromethoxy)methyl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1022 | (Rₐ)-2-amino-4-(7-chloro-2-((difluoromethoxy)methyl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1023 | (Sₐ)-2-amino-4-(7-chloro-2-((difluoromethoxy)methyl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1024 | 2-amino-4-(7-chloro-2-((2-cyanoethoxy)methyl)-9-fluoro-4-((3R,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1025 | 2-amino-4-(7-chloro-2-((2-cyanoethoxy)methyl)-9-fluoro-4-((3R,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1026 | 2-amino-4-(7-chloro-2-((2-cyanoethoxy)methyl)-9-fluoro-4-((3S,5S)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1027 | 2-amino-4-(7-chloro-2-((2-cyanoethoxy)methyl)-9-fluoro-4-((3S,5R)-1-(methyl-d₃)- |
| | 2-oxo-5-(trifluoromethyl)pyrrolidin-3-yl)-5,6-dihydro-4H-benzo[de][1,6]naphthyridin- |
| | 8-yl)-7-fluorobenzo[b]thiophene-3-carbonitrile |
| 1028 | (Rₐ)-2-amino-4-(7-chloro-2-((2-cyanoethoxy)methyl)-9-fluoro-4-((3R,5S)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1029 | (Sₐ)-2-amino-4-(7-chloro-2-((2-cyanoethoxy)methyl)-9-fluoro-4-((3R,5S)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1030 | (Rₐ)-2-amino-4-(7-chloro-2-((2-cyanoethoxy)methyl)-9-fluoro-4-((3S,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1031 | (Sₐ)-2-amino-4-(7-chloro-2-((2-cyanoethoxy)methyl)-9-fluoro-4-((3S,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1032 | (Rₐ)-2-amino-4-(7-chloro-2-((2-cyanoethoxy)methyl)-9-fluoro-4-((3R,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1033 | (Sₐ)-2-amino-4-(7-chloro-2-((2-cyanoethoxy)methyl)-9-fluoro-4-((3R,5R)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1034 | (Rₐ)-2-amino-4-(7-chloro-2-((2-cyanoethoxy)methyl)-9-fluoro-4-((3S,5S)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1035 | (Sₐ)-2-amino-4-(7-chloro-2-((2-cyanoethoxy)methyl)-9-fluoro-4-((3S,5S)-1-(methyl-d₃)- |
| | 2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1036 | 2-amino-4-(7-chloro-9-fluoro-2-(hydroxymethyl)-4-((3R,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1037 | 2-amino-4-(7-chloro-9-fluoro-2-(hydroxymethyl)-4-((3R,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1038 | 2-amino-4-(7-chloro-9-fluoro-2-(hydroxymethyl)-4-((3S,5S)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1039 | 2-amino-4-(7-chloro-9-fluoro-2-(hydroxymethyl)-4-((3S,5R)-1-(methyl-d₃)-2-oxo-5- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1040 | (Rₐ)-2-amino-4-(7-chloro-9-fluoro-2-(hydroxymethyl)-4-((3R,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1041 | (Sₐ)-2-amino-4-(7-chloro-9-fluoro-2-(hydroxymethyl)-4-((3R,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1042 | (Rₐ)-2-amino-4-(7-chloro-9-fluoro-2-(hydroxymethyl)-4-((3S,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1043 | (Sₐ)-2-amino-4-(7-chloro-9-fluoro-2-(hydroxymethyl)-4-((3S,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1044 | (Rₐ)-2-amino-4-(7-chloro-9-fluoro-2-(hydroxymethyl)-4-((3R,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1045 | (Sₐ)-2-amino-4-(7-chloro-9-fluoro-2-(hydroxymethyl)-4-((3R,5R)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1046 | (Rₐ)-2-amino-4-(7-chloro-9-fluoro-2-(hydroxymethyl)-4-((3S,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1047 | (Sₐ)-2-amino-4-(7-chloro-9-fluoro-2-(hydroxymethyl)-4-((3S,5S)-1-(methyl-d₃)-2- |
| | oxo-5-(trifluoromethyl)pyrrolidin-3-yl)-5,6-dihydro-4H-benzo[de][1,6]naphthyridin-8- |
| | yl)-7-fluorobenzo[b]thiophene-3-carbonitrile |
| 1048 | 2-amino-4-(7-chloro-2-((1r,3R)-3-(dimethylamino)cyclobutyl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1049 | 2-amino-4-(7-chloro-2-((1r,3R)-3-(dimethylamino)cyclobutyl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1050 | 2-amino-4-(7-chloro-2-((1r,3R)-3-(dimethylamino)cyclobutyl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1051 | 2-amino-4-(7-chloro-2-((1r,3R)-3-(dimethylamino)cyclobutyl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1052 | 2-amino-4-(7-chloro-2-((1s,3S)-3-(dimethylamino)cyclobutyl)-9-fluoro-4-((3R,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1053 | 2-amino-4-(7-chloro-2-((1s,3S)-3-(dimethylamino)cyclobutyl)-9-fluoro-4-((3R,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1054 | 2-amino-4-(7-chloro-2-((1s,3S)-3-(dimethylamino)cyclobutyl)-9-fluoro-4-((3S,5S)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1055 | 2-amino-4-(7-chloro-2-((1s,3S)-3-(dimethylamino)cyclobutyl)-9-fluoro-4-((3S,5R)-1- |
| | (methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1056 | (Rₐ)-2-amino-4-(7-chloro-2-((1r,3R)-3-(dimethylamino)cyclobutyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1057 | (Sₐ)-2-amino-4-(7-chloro-2-((1r,3R)-3-(dimethylamino)cyclobutyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1058 | (Rₐ)-2-amino-4-(7-chloro-2-((1r,3S)-3-(dimethylamino)cyclobutyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1059 | (Sₐ)-2-amino-4-(7-chloro-2-((1r,3S)-3-(dimethylamino)cyclobutyl)-9-fluoro-4- |
| | ((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1060 | (Rₐ)-2-amino-4-(7-chloro-2-((1r,3R)-3-(dimethylamino)cyclobutyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1061 | (Sₐ)-2-amino-4-(7-chloro-2-((1r,3R)-3-(dimethylamino)cyclobutyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1062 | (Rₐ)-2-amino-4-(7-chloro-2-((1r,3S)-3-(dimethylamino)cyclobutyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1063 | (Sₐ)-2-amino-4-(7-chloro-2-((1r,3S)-3-(dimethylamino)cyclobutyl)-9-fluoro-4- |
| | ((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1064 | 2-amino-4-(7-chloro-2-((3S,4S)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1065 | 2-amino-4-(7-chloro-2-((3S,4S)-3-(dimethylamino)-4-methoxypyrrolidin-1-yl)-9- |
| | fluoro-4-((3R,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1066 | 2-amino-4-(7-chloro-2-((3S,4S)-3-(dimethylamino)-4-methoxypyrrolidin-1-yl)-9- |
| | fluoro-4-((3S,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1067 | 2-amino-4-(7-chloro-2-((3S,4S)-3-(dimethylamino)-4-methoxypyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1068 | 2-amino-4-(7-chloro-2-((3R,4R)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1069 | 2-amino-4-(7-chloro-2-((3R,4R)-3-(dimethylamino)-4-methoxypyrrolidin-1-yl)-9- |
| | fluoro-4-((3R,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1070 | 2-amino-4-(7-chloro-2-((3R,4R)-3-(dimethylamino)-4-methoxypyrrolidin-1-yl)-9- |
| | fluoro-4-((3S,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1071 | 2-amino-4-(7-chloro-2-((3R,4R)-3-(dimethylamino)-4-methoxypyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1072 | (Rₐ)-2-amino-4-(7-chloro-2-((3S,4S)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1073 | (Sₐ)-2-amino-4-(7-chloro-2-((3S,4S)-3-(dimethylamino)-4-methoxypyrrolidin-1-yl)-9- |
| | fluoro-4-((3R,5S)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1074 | (Rₐ)-2-amino-4-(7-chloro-2-((3R,4R)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1075 | (Sₐ)-2-amino-4-(7-chloro-2-((3R,4R)-3-(dimethylamino)-4-methoxypyrrolidin-1-yl)- |
| | 9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1076 | (Rₐ)-2-amino-4-(7-chloro-2-((3S,4S)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1077 | (Sₐ)-2-amino-4-(7-chloro-2-((3S,4S)-3-(dimethylamino)-4-methoxypyrrolidin-1-yl)-9- |
| | fluoro-4-((3S,5R)-1-(methyl-d₃)-2-oxo-5-(trifluoromethyl)pyrrolidin-3-yl)-5,6-dihydro- |
| | 4H-benzo[de][1,6]naphthyridin-8-yl)-7-fluorobenzo[b]thiophene-3-carbonitrile |
| 1078 | (Rₐ)-2-amino-4-(7-chloro-2-((3R,4R)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1079 | (Sₐ)-2-amino-4-(7-chloro-2-((3R,4R)-3-(dimethylamino)-4-methoxypyrrolidin-1-yl)- |
| | 9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1080 | 2-amino-4-(7-chloro-2-(4-(dimethylamino)-2-azabicyclo[2.1.1]hexan-2-yl)-9-fluoro- |
| | 4-((3R,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1081 | 2-amino-4-(7-chloro-2-(4-(dimethylamino)-2-azabicyclo[2.1.1]hexan-2-yl)-9-fluoro- |
| | 4-((3R,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1082 | 2-amino-4-(7-chloro-2-(4-(dimethylamino)-2-azabicyclo[2.1.1]hexan-2-yl)-9-fluoro- |
| | 4-((3S,5S)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1083 | 2-amino-4-(7-chloro-2-(4-(dimethylamino)-2-azabicyclo[2.1.1]hexan-2-yl)-9-fluoro- |
| | 4-((3S,5R)-1-(methyl-d₃)-2-oxo-5-(trifluoromethyl)pyrrolidin-3-yl)-5,6-dihydro-4H- |
| | benzo[de][1,6]naphthyridin-8-yl)-7-fluorobenzo[b]thiophene-3-carbonitrile |
| 1084 | (Rₐ)-2-amino-4-(7-chloro-2-((1R,4R)-4-(dimethylamino)-2-azabicyclo[2.1.1]hexan- |
| | 2-yl)-9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1085 | (Sₐ)-2-amino-4-(7-chloro-2-((1R,4R)-4-(dimethylamino)-2-azabicyclo[2.1.1]hexan- |
| | 2-yl)-9-fluoro-4-((3R,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1086 | (Rₐ)-2-amino-4-(7-chloro-2-((1S,4S)-4-(dimethylamino)-2-azabicyclo[2.1.1]hexan- |
| | 2-yl)-9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1087 | (Sₐ)-2-amino-4-(7-chloro-2-((1S,4S)-4-(dimethylamino)-2-azabicyclo[2.1.1]hexan- |
| | 2-yl)-9-fluoro-4-((3S,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1088 | (Rₐ)-2-amino-4-(7-chloro-2-((1R,4R)-4-(dimethylamino)-2-azabicyclo[2.1.1]hexan- |
| | 2-yl)-9-fluoro-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1089 | (Sₐ)-2-amino-4-(7-chloro-2-((1R,4R)-4-(dimethylamino)-2-azabicyclo[2.1.1]hexan- |
| | 2-yl)-9-fluoro-4-((3S,5S)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1090 | (Rₐ)-2-amino-4-(7-chloro-2-((1R,4R)-4-(dimethylamino)-2-azabicyclo[2.1.1]hexan- |
| | 2-yl)-9-fluoro-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1091 | (Sₐ)-2-amino-4-(7-chloro-2-((1R,4R)-4-(dimethylamino)-2-azabicyclo[2.1.1]hexan- |
| | 2-yl)-9-fluoro-4-((3R,5R)-1-(methyl-d₃)-2-oxo-5-(trifluoromethyl)pyrrolidin-3-yl)-5,6- |
| | dihydro-4H-benzo[de][1,6]naphthyridin-8-yl)-7-fluorobenzo[b]thiophene-3-carbonitrile |
| 1092 | 2-amino-4-(7-chloro-4-((R)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1093 | 2-amino-4-(7-chloro-4-((S)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1094 | (Rₐ)-2-amino-4-(7-chloro-4-((R)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1095 | (Sₐ)-2-amino-4-(7-chloro-4-((R)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1096 | (Rₐ)-2-amino-4-(7-chloro-4-((S)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1097 | (Sₐ)-2-amino-4-(7-chloro-4-((S)-1-ethyl-2-oxopyrrolidin-3-yl)-9-fluoro-2-methyl-5,6- |
| | dihydro-4H-benzo[de][1,6]naphthyridin-8-yl)-7-fluorobenzo[b]thiophene-3-carbonitrile |
| 1098 | 2-amino-4-(7-chloro-9-fluoro-4-((R)-1-(isothiazol-4-yl)ethyl)-2-methyl-5,6-dihydro- |
| | 4H-benzo[de][1,6]naphthyridin-8-yl)-7-fluorobenzo[b]thiophene-3-carbonitrile |
| 1099 | 2-amino-4-(7-chloro-9-fluoro-4-((S)-1-(isothiazol-4-yl)ethyl)-2-methyl-5,6-dihydro- |
| | 4H-benzo[de][1,6]naphthyridin-8-yl)-7-fluorobenzo[b]thiophene-3-carbonitrile |
| 1100 | (Rₐ)-2-amino-4-(7-chloro-9-fluoro-4-((R)-1-(isothiazol-4-yl)ethyl)-2-methyl-5,6- |
| | dihydro-4H-benzo[de][1,6]naphthyridin-8-yl)-7-fluorobenzo[b]thiophene-3-carbonitrile |
| 1101 | (Sₐ)-2-amino-4-(7-chloro-9-fluoro-4-((R)-1-(isothiazol-4-yl)ethyl)-2-methyl-5,6- |
| | dihydro-4H-benzo[de][1,6]naphthyridin-8-yl)-7-fluorobenzo[b]thiophene-3-carbonitrile |
| 1102 | (Rₐ)-2-amino-4-(7-chloro-9-fluoro-4-((S)-1-(isothiazol-4-yl)ethyl)-2-methyl-5,6- |
| | dihydro-4H-benzo[de][1,6]naphthyridin-8-yl)-7-fluorobenzo[b]thiophene-3-carbonitrile |
| 1103 | (Sₐ)-2-amino-4-(7-chloro-9-fluoro-4-((S)-1-(isothiazol-4-yl)ethyl)-2-methyl-5,6- |
| | dihydro-4H-benzo[de][1,6]naphthyridin-8-yl)-7-fluorobenzo[b]thiophene-3-carbonitrile |
| 1104 | 2-amino-4-(7-chloro-9-fluoro-2-methyl-4-((3R,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1105 | 2-amino-4-(7-chloro-9-fluoro-2-methyl-4-((3R,5S)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1106 | 2-amino-4-(7-chloro-9-fluoro-2-methyl-4-((3S,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1107 | 2-amino-4-(7-chloro-9-fluoro-2-methyl-4-((3S,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1108 | (Rₐ)-2-amino-4-(7-chloro-9-fluoro-2-methyl-4-((3R,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1109 | (Sₐ)-2-amino-4-(7-chloro-9-fluoro-2-methyl-4-((3R,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1110 | (Rₐ)-2-amino-4-(7-chloro-9-fluoro-2-methyl-4-((3,5S)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1111 | (Sₐ)-2-amino-4-(7-chloro-9-fluoro-2-methyl-4-((3S,5S)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1113 | (Rₐ)-2-amino-4-(7-chloro-9-fluoro-2-methyl-4-((3R,5S)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1114 | (Sₐ)-2-amino-4-(7-chloro-9-fluoro-2-methyl-4-((3R,5S)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1115 | (Rₐ)-2-amino-4-(7-chloro-9-fluoro-2-methyl-4-((3S,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1116 | (Sₐ)-2-amino-4-(7-chloro-9-fluoro-2-methyl-4-((3S,5R)-5-methyl-1-(methyl-d₃)-2- |
| | oxopyrrolidin-3-yl)-5,6-dihydro-4H-benzo[de][1,6]naphthyridin-8-yl)-7- |
| | fluorobenzo[b]thiophene-3-carbonitrile |
| 1117 | 2-amino-4-(7-chloro-4-((3R,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1118 | 2-amino-4-(7-chloro-4-((3R,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1119 | 2-amino-4-(7-chloro-4-((3S,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1120 | 2-amino-4-(7-chloro-4-((3S,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1121 | (Rₐ)-2-amino-4-(7-chloro-4-((3R,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1122 | (Sₐ)-2-amino-4-(7-chloro-4-((3R,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1123 | (Rₐ)-2-amino-4-(7-chloro-4-((3S,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1124 | (Sₐ)-2-amino-4-(7-chloro-4-((3S,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1125 | (Rₐ)-2-amino-4-(7-chloro-4-((3R,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1126 | (Sₐ)-2-amino-4-(7-chloro-4-((3R,4R)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1127 | (Rₐ)-2-amino-4-(7-chloro-4-((3S,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1128 | (Sₐ)-2-amino-4-(7-chloro-4-((3S,4S)-1,4-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1129 | 2-amino-4-(7-chloro-4-((3R,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1130 | 2-amino-4-(7-chloro-4-((3S,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1131 | 2-amino-4-(7-chloro-4-((3R,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1132 | 2-amino-4-(7-chloro-4-((3S,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1133 | (Rₐ)-2-amino-4-(7-chloro-4-((3R,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1134 | (Sₐ)-2-amino-4-(7-chloro-4-((3R,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1135 | (Rₐ)-2-amino-4-(7-chloro-4-((3S,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1136 | (Sₐ)-2-amino-4-(7-chloro-4-((3S,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1137 | (Rₐ)-2-amino-4-(7-chloro-4-((3R,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1138 | (Sₐ)-2-amino-4-(7-chloro-4-((3R,5S)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1139 | (Rₐ)-2-amino-4-(7-chloro-4-((3S,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1140 | (Sₐ)-2-amino-4-(7-chloro-4-((3S,5R)-1,5-dimethyl-2-oxopyrrolidin-3-yl)-9-fluoro-2- |
| | methyl-5,6-dihydro-4H-benzo[de][1,6]naphthyridin-8-yl)-7-fluorobenzo[b]thiophene- |
| | 3-carbonitrile |
| 1141 | 2-amino-4-(7-chloro-9-fluoro-2-methyl-4-((R)-2-oxo-1-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 1142 | 2-amino-4-(7-chloro-9-fluoro-2-methyl-4-((S)-2-oxo-1-(trifluoromethyl)pyrrolidin-3- |
| | yl)-5,6-dihydro-4H-benzo[de][1,6]naphthyridin-8-yl)-7-fluorobenzo[b]thiophene-3- |
| | carbonitrile |
| 1143 | (Rₐ)-2-amino-4-(7-chloro-9-fluoro-2-methyl-4-((R)-2-oxo-1- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1144 | (Sₐ)-2-amino-4-(7-chloro-9-fluoro-2-methyl-4-((R)-2-oxo-1- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1445 | (Rₐ)-2-amino-4-(7-chloro-9-fluoro-2-methyl-4-((S)-2-oxo-1- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |
| 1446 | (Sₐ)-2-amino-4-(7-chloro-9-fluoro-2-methyl-4-((S)-2-oxo-1- |
| | (trifluoromethyl)pyrrolidin-3-yl)-5,6-dihydro-4H-benzo[de][1,6]naphthyridin-8-yl)-7- |
| | fluorobenzo[b]thiophene-3-carbonitrile |

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule.

The term “Cₙ₋ₘ” indicates a range which includes the endpoints, wherein n and m are integers and indicate the number of carbons present in a chemical moiety. The term is intended to include each and every member in the indicated range. Thus, Cₙ₋ₘ includes each member in the series Cₙ, Cₙ₊₁, . . . Cₘ₋₁, and Cₘ. Examples include C₁₋₄ (which includes C₁, C₂, C₃, and C₄), C₁₋₆ (which includes C₁, C₂, C₃, C₄, C₅, and C₆) and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkylenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “oxo” or “oxy” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “COₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl” or “cycloalkane” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” or “Cₙ₋ₘ cycloalkane” refers to a cycloalkyl or cycloalkane that has n to m ring member carbon atoms. Cycloalkyl groups can include mono- or polycyclic (e.g., having 2, 3 or 4 fused rings) groups and spirocycles. In the following, references to cycloalkyl groups apply equally to cycloalkane groups. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl. In some embodiments, the cycloalkyl group is tetrahydronaphthalenyl (e.g., 1,2,3,4-tetrahydronaphthalenyl).

The term “heterocycloalkyl” or “heterocycloalkane” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. The term “n-m-membered heterocycloalkyl” or “n-m-membered heterocycloalkane” where n and m are integers refer to a heterocycloalkyl or heterocycloalkane ring or ring system containing from n to m ring-forming atoms. An n-m-membered heterocycloalkyl or heterocycloalkane includes from 1 to m−1 carbon atoms and from 1 to m−1 heteroatoms. The term “n-membered heterocycloalkyl” or “n-membered heterocycloalkane” where n is an integer refers to a heterocycloalkyl ring or ring system containing from n to m ring-forming atoms. In the following, references to heterocycloalkyl groups, rings or ring systems apply equally to heterocycloalkane groups, rings or ring systems. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazobicyclo[2.2.1]heptanyl; pyrrolidinyl; hexahydropyrrolo[3,4-b]pyrrol-1 (2H)-yl; 1,6-dihydropyridinyl; morpholinyl; azetidinyl; piperazinyl; and 4,7-diazaspiro[2.5]octan-7-yl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereoisomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

When the compounds described herein contain a chiral center, unless otherwise indicated, the compounds can be any of the possible stereoisomers. In some embodiments, the compounds provided herein have the (R)-configuration. In other embodiments, the compounds have the (S)-configuration. In compounds with more than one chiral centers, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

Compounds provided herein can exist in the form of atropisomers (i.e., conformational diastereoisomers) that can be stable at ambient temperature and separable, e.g., by chromatography. For example, the compounds of Formula I can exist in the form of atropisomers that are interchangeable by rotation around the bond connecting Cy¹ (or any of the embodiments thereof) to the remainder of the molecule. Reference to the compounds described herein or any of the embodiments is understood to include all such atropisomeric forms of the compounds. Without being limited by any theory, it is understood that, for a given compound, one atropisomer may be more potent as an inhibitor of KRAS (including G12D mutated form of KRAS) than another atropisomer. For example, compounds of Formula I as described herein in which Cy¹ is 2,3-dichlorophenyl can exist in the form of atropisomers in which the conformation of the dichlorophenyl relative to the remainder of the molecule is as shown by the partial formulae Formula A or Formula B below. Atropisomeric forms may be isolable by methods such as chromatography. The stereochemistry of atropisomeric forms can be designated, e.g., as either Rₐ or Sₐ applying IUPAC rules of nomenclature for stereochemistry. G. P. Moss, Pure & Appl. Chem., 1996, 68(12), 2193-2222. Without being limited by any theory, it is understood that, for a given compound, the atropisomer represented by Formula (A) is generally more potent as an inhibitor of KRAS (including G12C, G12D or G12V mutated forms of KRAS) than the atropisomer represented by Formula (B). In some embodiments, an atropisomer can be least partially or substantially separated from the alternative atropisomer of the compound, for example containing about 40% or less, about 30% or less, about 20% or less, about 10% or less, about 5% or less, about 2% or less, or about 1% or less of the alternative atropisomer.

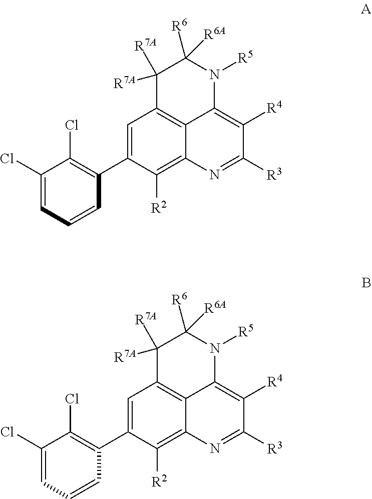

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. In the compounds provided herein, any atom not specifically designated as a particular isotope is meant to represent any stable isotope of that atom.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med. Chem. 2011, 54, 201-210; R. Xu et. al. J. Label Compd. Radiopharm. 2015, 58, 308-312). Unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium). In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “compound” is intended, unless otherwise specified, to include all stereoisomers, including without limitation, geometric isomers, configurational isomers, conformational isomers, rotational isomers, and atropisomers, of the structures depicted, including each of the embodiments thereof. The term is also intended to refer to compounds described herein regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds provided herein, or salts thereof, are substantially isolated. “Substantially isolated” means that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions “ambient temperature” and “room temperature” are understood in the art, and refer generally to a temperature, e.g., a reaction temperature, that is about the temperature of the room in which the reaction is carried out, e.g., a temperature from about 20° C. to about 30° C., or about 25° C.

The present disclosure also includes pharmaceutically acceptable salts of the compounds described herein, including any of the embodiments thereof. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011). In some embodiments, the compounds described herein include the N-oxide forms.

### II. Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high-performance liquid chromatography (H PLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

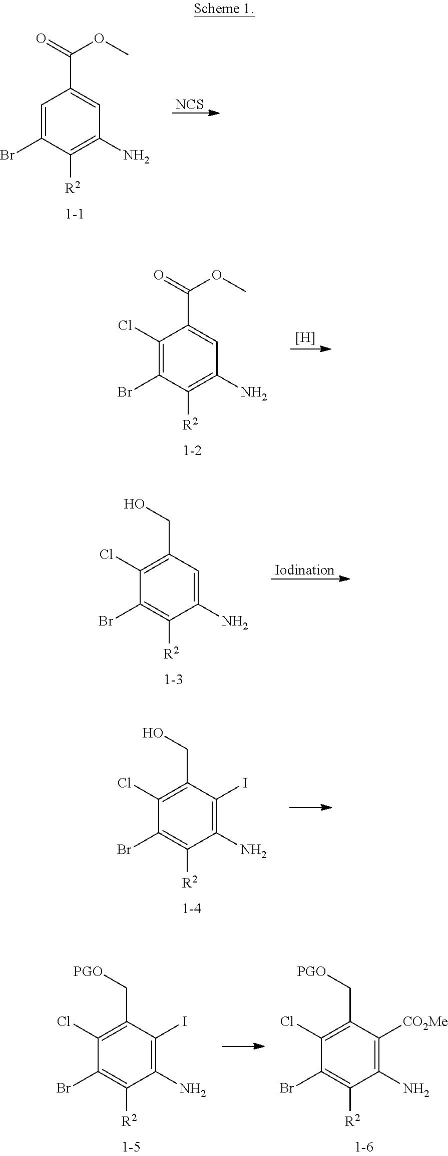

Compounds of the invention can be prepared via key intermediate 1-6, the synthetic route for which is outlined in Scheme 1. Chlorination of compound 1-1 with an appropriate reagent, such as N-chlorosuccinimide (NCS), affords intermediate 1-2. Compound 1-3 can be prepared by treating 1-2 with a reducing reagent such as LiBH₄. Iodination of compound 1-3 with an appropriate reagent, such as N-iodosuccinimide (NIS) or iodine/silver sulfate affords intermediate 1-4. Intermediate 1-4 can undergo a hydroxyl protection to deliver compound 1-5. Finally, formylation under CO gas and an appropriate palladium catalyst provides ester 1-6.

Staring from compound 1-6, key compounds can be prepared according to the route outlined in Scheme 2. Acetylation of the free amine proves compound 2-1, which can be converted to the corresponding methyl ketone 2-2 through a four step sequence involving ester reduction to the corresponding alcohol, oxidation to the corresponding aldehyde, methylation with a metal-methyl reagent such as methyl magnesium bromide, and final oxidation to the corresponding methyl ketone. Cyclization of the resulting intermediate can be achieved by treatment with the appropriate base, such as NaOtBu, with concominant deprotection of the protecting group, to provide 2-3. Double chlorination of 2-3 with an appropriate chlorinating agent, such as POCl₃, provides 2-4, which in turn can undergo displacement with a cyanide salt, such as NaCN, followed by reduction with an appropriate metal hydride, such as DIBAL-H, to provide aldehyde 2-5. Reductive amination conditions using an appropriate reducing agent, such as NaBH(OAc)₃, in the presence or absence of an appropriate acid, such as TFA, provides 2-6 after in situ cyclization.

Finally, cross coupling with an appropriate metallated Cy¹-M, such as a boronic acid, boronic ester or aminoheterocycle, under standard Suzuki Cross-Coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a pallidum catalyst), or standard Buchwald-Hartwig amination conditions (e.g., in the presence of a pallidum catalyst) provides the final products of general formula 2-7. The order of the above described chemical reactions can be rearranged as appropriate to suite the preparation of different analogues.

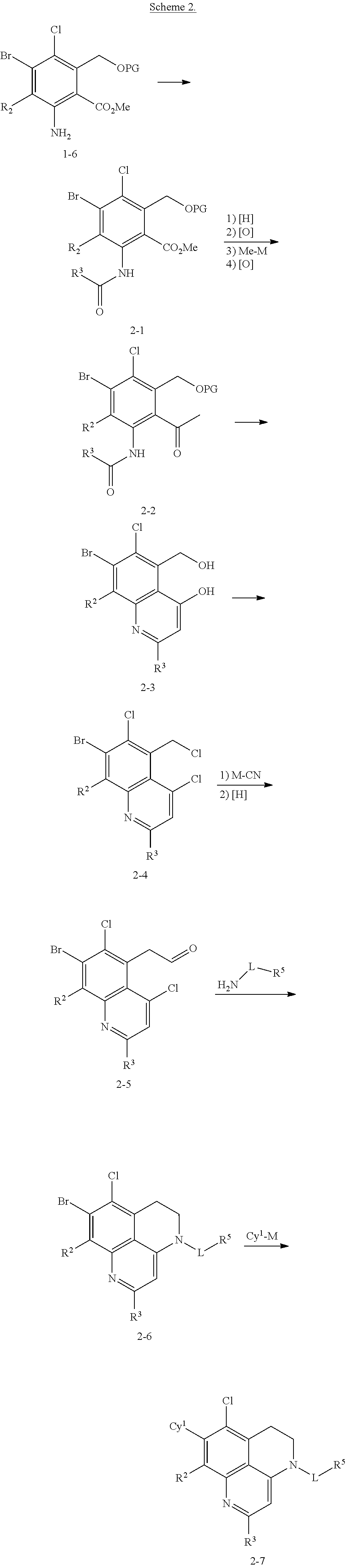

Alternatively, compounds described herein can be prepared according to the general route outlined in Scheme 3.

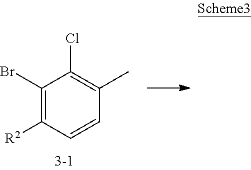

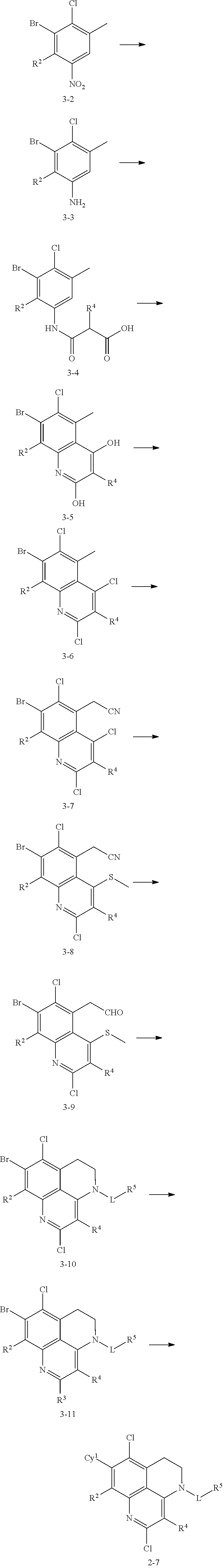

Starting material 3-1 can be regioselectively nitrated using a variety of nitrating reagents, such as tert-butyl nitrite, to provide intermediate 3-2. Subsequent reduction under a variety of conditions, such as with iron, provides aniline 3-3. Condensation with an appropriately substituted dimalonate followed by hydrolysis provides intermediate 3-4. Cyclization, mediated by e.g. Eaton's reagent, provides cyclic intermediate 3-5 Chlorination with a suitable chlorinating agent such as phosphorus oxychloride provides intermediate 3-6. Intermediate 3-6 undergoes smooth radical bromination at the benzylic position mediated by a radical initiator such as AIBN and an appropriate bromine source, such as NBS or elemental bromine, followed by nucleophilic displacement with an appropriate metal cyanide source under phase transfer conditions to provide 3-7. Displacement with sodium thiomethoxide then provides intermediate 3-8, which can be reduced by an appropriate reducing agent such as DIBAL-H to provide intermediate 3-9. Reductive amination between intermediate 3-9 and an appropriate amine under acidic conditions and an appropriate reducing agent such as NaBH₃CN proceeds with optional concominant cyclization to provide intermediate 3-10. The R³ substituent can then be installed via nucleophilic displacement mediated by an appropriate base or under a variety of metal-mediated conditions, including Suzuki cross coupling with an appropriate boronic acid or ester, Stille cross coupling with an appropriate stannane, Negishi coupling with an appropriate zincate and carbonylative coupling in the presence of carbon monoxide to provide intermediates of general structure 3-11. Installation of Cy¹ as described in Scheme 2 provides compounds of general formula 2-7.

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St. C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### III. Uses of the Compounds

Compounds of the present disclosure, including the compounds of Formula (I), or any of the embodiments thereof, are useful for therapy as described in further detail below. The present disclosure provides compounds of Formula (I), for use as a medicament, or for use in medicine. The present disclosure provides compounds of Formula (I), for use as a medicament, or for use in treating disease, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure are KRAS inhibitors and, thus, are useful in treating diseases and disorders associated with activity of KRAS. For the uses described herein, any of the compounds of Formula (I), including any of the embodiments thereof, may be used.

In particular, compounds of the invention are KRAS inhibitors having activity against one or more mutant forms of KRAS, and, thus, are useful in treating diseases and disorders associated with the presence or activity of mutant forms of KRAS, such as G12C, G12D, and/or the G12V mutant forms of KRAS.

The Ras family is comprised of three members: KRAS, NRAS and HRAS. RAS mutant cancers account for about 25% of human cancers. KRAS is the most frequently mutated isoform in human cancers: 85% of all RAS mutations are in KRAS, 12% in NRAS, and 3% in HRAS (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residues/codons 12, 13, and 61; Codon 12 mutations are most frequent in KRAS. The frequency of specific mutations varied between RAS genes and G12D mutations are most predominant in KRAS whereas Q61R and G12R mutations are most frequent in NRAS and HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas (nearly half of mutant KRAS is G12C), as well as 2-5% of pancreatic and colorectal adenocarcinomas, respectively (A. D. Cox, et al., Nat. Rev. Drug. Discov., 2014, 13(11), 828-51). Using shRNA knockdown thousands of genes across hundreds of cancer cell lines, genomic studies have demonstrated that cancer cells exhibiting KRAS mutations are highly dependent on KRAS function for cell growth (R. McDonald, et al., Cell, 2017, 170(3), 577-92).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Diseases that can be treated with the compounds of Formula (I) include cancers. The cancers can include adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. In some embodiments, the cancer can be adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, or Wilms' tumor.

The cancer types in which KRAS harboring G12C, G12V and G12D mutations are implicated and that can be treated using compounds of Formula (I), or any of the embodiments thereof, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (K. Knickelbein, et al., Genes Dis., 2015, 2(1), 4-12). KRAS mutations were found in immunological and inflammatory disorders (A. Fernandez-Medarde, et al., Genes Cancer, 2011, 2(3), 344-358) such as Ras-associated lymphoproliferative disorder (RALD) or juvenile myelomonocytic leukemia (JMML) caused by somatic mutations of KRAS or NRAS.

Compounds of the present disclosure, including any of the embodiments thereof, can inhibit the activity of the KRAS protein. For example, compounds of the present disclosure can be used to inhibit activity of KRAS in a cell or in an individual or patient in need of inhibition of the enzyme by administering an inhibiting amount of one or more compounds of the present disclosure to the cell, individual, or patient.

As KRAS inhibitors, the compounds of the present disclosure, or any of the embodiments thereof, are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, the method comprising contacting a compound of the instant disclosure with KRAS. In an embodiment, the contacting comprises administering the compound to a patient. In an embodiment, KRAS is characterized as having a somatic mutation of G12C. In another embodiment, KRAS is characterized as having a somatic mutation of G12D. In another embodiment, KRAS is characterized as having a somatic mutation of G12V.

In an aspect, provided herein is a method of inhibiting a KRAS protein harboring a G12C mutation, the method comprising contacting a compound of Formula (I), or any of the embodiments thereof, with KRAS.

In an aspect, provided herein is a method of inhibiting a KRAS protein harboring a G12D mutation, the method comprising contacting a compound of Formula (I), or any of the embodiments thereof, with KRAS harboring a G12D mutation.

In an aspect, provided herein is a method of inhibiting a KRAS protein harboring a G12V mutation, the method comprising contacting a compound of Formula (I), or any of the embodiments thereof, with KRAS harboring a G12V mutation.

In another aspect, provided herein is a method of treating a disease or disorder associated with inhibition of KRAS interaction, the method comprising administering to a patient in need thereof a therapeutically effective amount of a compound of Formula (I), or any of the embodiments thereof.

In an embodiment, the disease or disorder is an immunological or inflammatory disorder. In another embodiment, the immunological or inflammatory disorder is Ras-associated lymphoproliferative disorder or juvenile myelomonocytic leukemia caused by somatic mutations of KRAS. In an embodiment, the immunological or inflammatory disorder is caused by a somatic mutation of KRAS.

In another embodiment, the somatic mutation of KRAS is G12C. In another embodiment, the somatic mutation of KRAS is G12D. In another embodiment, the somatic mutation of KRAS is G12V.

In another embodiment, the immunological or inflammatory disorder is associated with activity of KRAS having a G12C mutation. In another embodiment, the immunological or inflammatory disorder is associated with activity of KRAS having a G12D mutation. In another embodiment, the immunological or inflammatory disorder is associated with activity of KRAS having a G12V mutation.

In yet another aspect, provided herein is a method of treating a disease or disorder associated with inhibiting a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with inhibiting a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with inhibiting a KRAS protein harboring a G12V mutation, the method comprising administering to a patient in need thereof a therapeutically effective amount of a compound of Formula (I), or any of the embodiments thereof.

In yet another aspect, provided herein is a method of treating a disease or disorder associated with activity of a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with activity of a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with activity of a KRAS protein harboring a G12V mutation, the method comprising administering to a patient in need thereof a therapeutically effective amount of a compound of Formula (I), or any of the embodiments thereof.

In another aspect, provided herein is also a method of treating cancer in a patient in need thereof comprising administering to the patient a therapeutically effective amount of a compound of Formula (I), or any of the embodiments thereof.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12C mutation.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12D mutation.

In another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12V mutation.

In yet another aspect, provided herein is a method for treating a cancer in a patient, the method comprising administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12C mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12D mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12V mutation, and administering to the patient a therapeutically effective amount of any one of the compounds disclosed herein, or pharmaceutically acceptable salt thereof.

In an embodiment, the cancer is selected from carcinomas, hematological cancers, sarcomas, and glioblastoma. In another embodiment, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet another embodiment, the carcinoma is selected from pancreatic, colorectal, lung, bladder, gastric, esophageal, breast, head and neck, cervical, skin, and thyroid. In another embodiment, the cancer is colorectal cancer, pancreatic cancer, or lung cancer. In yet another embodiment, the pancreatic cancer is pancreatic ductal adenocarcinoma (PDAC). In still another embodiment, the cancer is non-small cell lung cancer (NSCLC).

In an embodiment, the cancer is metastatic.

In an aspect, provided herein is a method for treating a disease or disorder associated with inhibition of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an aspect, provided herein is a method for treating a disease or disorder associated with activity of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In certain embodiments, the disclosure provides a method for treating a KRAS-mediated disorder in a patient in need thereof, comprising the step of administering to the patient a compound according to the invention, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastasis.

In addition to oncogenic neoplasms, the compounds of the invention are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

The term “8p11 myeloproliferative syndrome” refers to myeloid/lymphoid neoplasms associated with eosinophilia and abnormalities of FGFR1.

The term “cell” refers to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” KRAS with a compound described herein includes the administration of a compound described herein to an individual or patient, such as a human, having KRAS, as well as, for example, introducing a compound described herein into a sample containing a cellular or purified preparation containing KRAS.

The terms “individual,” “subject,” or “patient,” are used interchangeably, and refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” comprises the prevention of at least one symptom associated with or caused by the state, disease or disorder being prevented.

### III. Combination Therapies

Compounds of the present disclosure, including the compounds of Formula (I), or any of the embodiments thereof, may be useful in therapy when used in combination with one or more additional pharmaceutical agents, as described in further detail below.

a. Cancer Therapies

Compounds of the invention, including the compounds of Formula (I), or any of the embodiments thereof, may be useful in treatment of cancer when used in combination with one or more additional pharmaceutical agents, as described in further detail below.

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of KRAS-associated diseases, disorders or conditions. Other agents such as therapeutic antibodies can be used in combination with the compounds of the present disclosure for treatment of KRAS-associated diseases, disorders or conditions. The one or more additional pharmaceutical agents can be administered to a patient simultaneously or sequentially.

In some embodiments, the KRAS inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2; e.g., ruxolitinib or baricitinib; or JAK1; e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872, or INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., zilurgisertib); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™ e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-1 agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, reylimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Pat. No. 7,745,437.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the embodiments thereof as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the embodiments thereof as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, Chlamydia, rickettsial bacteria, mycobacteria, staphylococci, streptococci, pneumococci, meningococci and conococci, Klebsiella, Proteus, Serratia, Pseudomonas, Legionella, diphtheria, Salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

b. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217,149, or 10,308,644; U.S. Publ. Nos. 2017/1145025, 2017/11746711, 2017/1174679, 2017/0320875, 2017/0342060, 2017/0362253, 2018/0016260, 2018/0057486, 2018/1177784, 2018/1177870, 2018/1179179, 2018/11792011, 2018/1179202, 2018/0273519, 2019/0040082, 2019/0062345, 2019/0071439, 2019/1127467, 2019/1144439, 2019/0202824, 2019/0225601, 2019/0300524, or 2019/0345170; or POT Pub. Nos. WO 03042402, WO 2008156712, WO02010089411, WO 2010036959, WO 2011066342, WO 2011159877, WO 20111082400, or WO 20111161699, which are each incorporated herein by reference in their entirety. In some embodiments, the inhibitor of PD-L1 is INC1B086550.

In some embodiments, the PD-L1 inhibitor is selected from the compounds in Table 3, or a pharmaceutically acceptable salt thereof.

TABLE 3

| | US | |
|---|---|---|
| | Publica- | |
| Cmpd | tion | |
| No. | Appl. No. | Name and Structure |
| 1 | US 2018- | (R)-1-((7-cyano-2-(3′-(3-(((R)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #24 | yl)methyl)pyrrolidine-3-carboxylic acid |
| | | 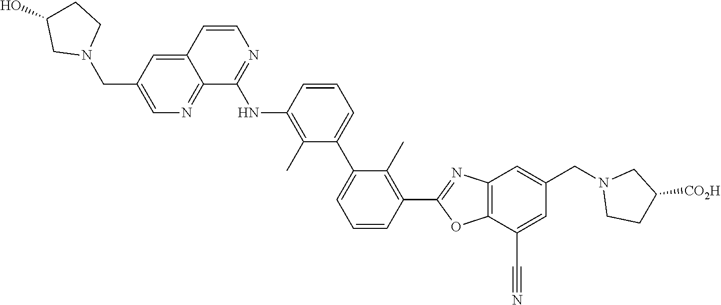 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 2 | US 2018- | N-(2-chloro-3′-(8-chloro-6-((2-hydroxyethylamino)methyl)-[1,2,4]triazolo[1,5- |
| | 0179201, | alpyridin-2-yl)-2′-methylbiphenyl-3-yl)-5-((2- |
| | Example #2 | hydroxyethylamino)methyl) picolinamide 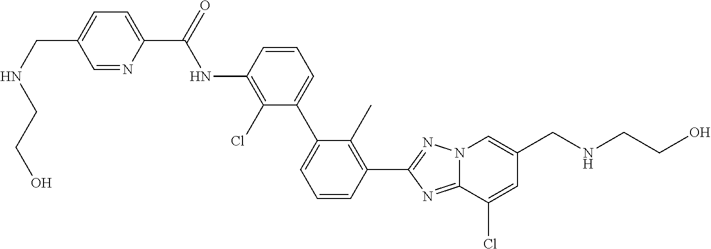 [SMILES: Cc1c(-c2nc3c(Cl)cc(CNCCO)cn3n2)cccc1-c1cccc(NC(=O)c2ccc(CNCCO)cn2)c1Cl] |
| 3 | US 2018- | (S)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #25 | yl)methyl)pyrrolidine-3-carboxylic acid 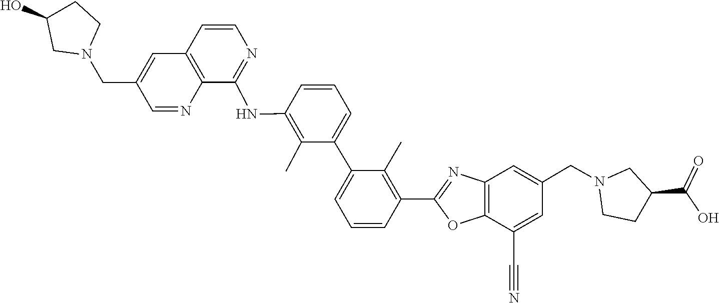 [SMILES: Cc1c(Nc2nccc3cc(CN4CC[C@H](O)C4)cnc23)cccc1-c1cccc(-c2nc3cc(CN4CC[C@H](C(=O)O)C4)cc(C#N)c3o2)c1C] |
| 4 | US 2018- | (R)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #26 | yl)methyl)pyrrolidine-3-carboxylic acid 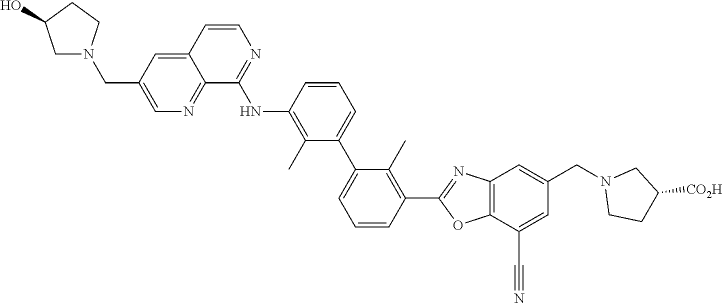 [SMILES: Cc1c(Nc2nccc3cc(CN4CC[C@H](O)C4)cnc23)cccc1-c1cccc(-c2nc3cc(CN4CC[C@@H](C(=O)O)C4)cc(C#N)c3o2)c1C] |
| 5 | US 2018- | (S)-1-((7-cyano-2-(3′-(3-(((R)-3-hydroxypyrrolidin-1-yl)methyl)-1,7- |
| | 0179197, | naphthyridin-8-ylamino)-2,2'-dimethylbiphenyl-3-yl)benzo[d]oxazol-5- |
| | Example #28 | yl)methyl)pyrrolidine-3-carboxylic acid 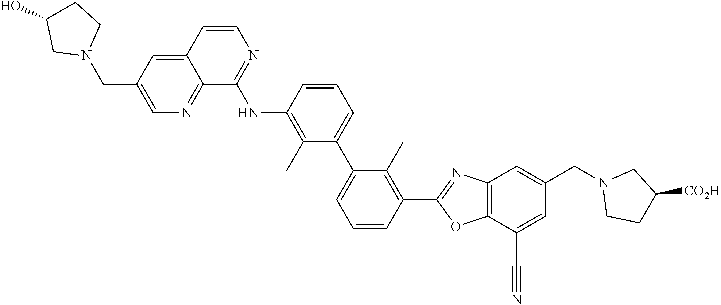 [SMILES: Cc1c(Nc2nccc3cc(CN4CC[C@@H](O)C4)cnc23)cccc1-c1cccc(-c2nc3cc(CN4CC[C@H](C(=O)O)C4)cc(C#N)c3o2)c1C] |
| 6 | US 2018- | 1-((7-cyano-2-(3′-(5-(2-(dimethylamino)acetyl)-5,6-dihydro-4H-pyrrolo[3,4- |
| | 0179197, | d]thiazol-2-yl)-2,2′-dimethylbiphenyl-3-yl)benzo[d]oxazol-5- |
| | Example #236 | yl)methyl)piperidine-4-carboxylic acid 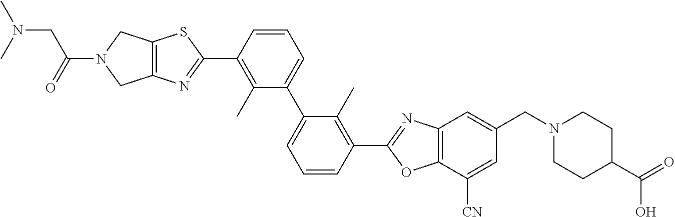 [SMILES: Cc1c(-c2nc3cc(CN4CCC(C(=O)O)CC4)cc(C#N)c3o2)cccc1-c1cccc(-c2nc3c(s2)CN(C(=O)CN(C)C)C3)c1C] |
| 7 | US 2018- | N,N-(2-chloro-2′-methylbiphenyl-3,3′-diyl)bis(5-((2-hydroxyethylamino) |
| | 0179179, Example #1 | methyl)picolinamide) 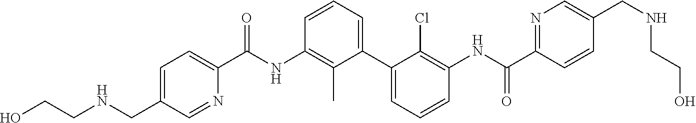 [SMILES: Cc1c(NC(=O)c2ccc(CNCCO)cn2)cccc1-c1cccc(NC(=O)c2ccc(CNCCO)cn2)c1Cl] |
| 8 | US 2018- | (R)-1-((6-(2′-chloro-3′-(5-((3-hydroxypyrrolidin-1-yl)methyl)picolinamido)-2- |
| | 0179179, Example #9 | methylbiphenyl-3-ylcarbamoyl)pyridin-3-yl)methyl)piperidine-4-carboxylic acid 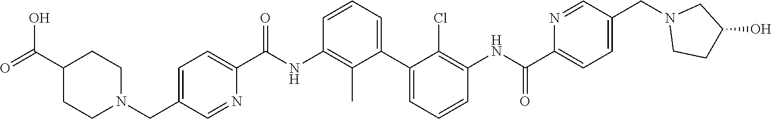 [SMILES: Cc1c(NC(=O)c2ccc(CN3CCC(C(=O)O)CC3)cn2)cccc1-c1cccc(NC(=O)c2ccc(CN3CC[C@@H](O)C3)cn2)c1Cl] |
| 9 | US 2018- | (S)-1-((6-((2′-chloro-2-methyl-3′-(5-(pyrrolidin-1-ylmethyl)picolinamido)-[1,1′- |
| | 0179179, | biphenyl]-3-yl)carbamoyl)-4-methylpyridin-3-yl)methyl)piperidine-2-carboxylic |
| | Example #12 | acid 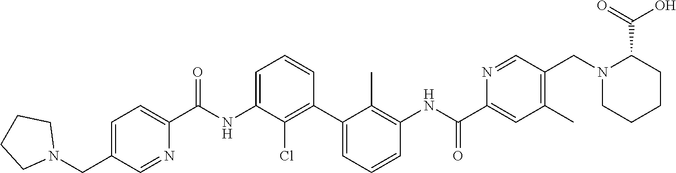 [SMILES: Cc1cc(C(=O)Nc2cccc(-c3cccc(NC(=O)c4ccc(CN5CCCC5)cn4)c3Cl)c2C)ncc1CN1CCCC[C@H]1C(=O)O] |
| 10 | US 2018- | trans 4-(2-(2-(2-chloro-3′-(3-(((R)-3-hydroxypyrrolidin-1-yl)methyl)-1,7- |
| | 0179202, | naphthyridin-8-ylamino)-2′-methylbiphenyl-3-ylcarbamoyl)-1-methyl-6,7- |
| | Example #52 | dihydro-1H-imidazo[4,5-c]pyridin-5(4H)-yl)ethyl)cyclohexanecarboxylic acid 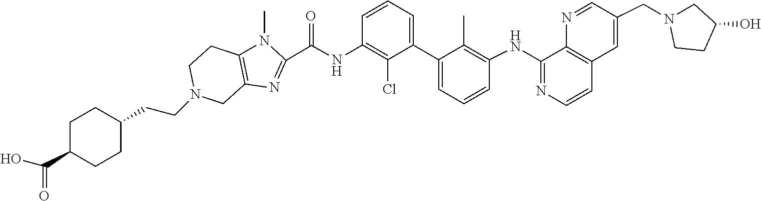 [SMILES: Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl] |
| 11 | US 2018- | cis-4-((2-(2-chloro-3′-(3-(((R)-3-hydroxy-3-methylpyrrolidin-1-yl)methyl)-1,7- |
| | 0179202, | naphthyridin-8-ylamino)-2′-methylbiphenyl-3-ylcarbamoyl)-1-methyl-6,7- |
| | Example #56 | dihydro-1H-imidazo[4,5-c]pyridin-5(4H)-yl)methyl)cyclohexanecarboxylic acid 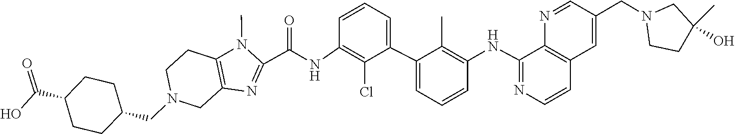 [SMILES: Cc1c(Nc2nccc3cc(CN4CC[C@@](C)(O)C4)cnc23)cccc1-c1cccc(NC(=O)c2nc3c(n2C)CCN(C[C@H]2CC[C@@H](C(=O)O)CC2)C3)c1Cl] |
| 12 | US 2018- | (R)-4-(2-(2-chloro-3′-(7-((3-hydroxypyrrolidin-1-yl)methyl)pyrido[3,2- |
| | 0179202, | d]pyrimidin-4-ylamino)-2′-methylbiphenyl-3-ylcarbamoyl)-1-methyl-6,7- |
| | Example | dihydro-1H-imidazo[4,5-c]pyridin-5(4H)-yl)-1-methylcyclohexanecarboxylic |
| | #68 | acid |
| | | 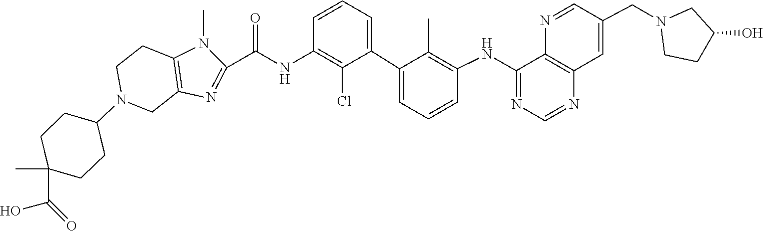 [SMILES: Cc1c(Nc2ncnc3cc(CN4CC[C@@H](O)C4)cnc23)cccc1-c1cccc(NC(=O)c2nc3c(n2C)CCN(C2CCC(C)(C(=O)O)CC2)C3)c1Cl] |
| 13 | US 2018- | (R)-1-((8-((2-chloro-3′-(5-(N-ethyl-N-methylglycyl)-5,6-dihydro-4H-pyrrolo[3,4- |
| | 0179202, | d]thiazol-2-yl)-2′-methyl-[1,1′-biphenyl]-3-yl)amino)-1,7-naphthyridin-3- |
| | Example #90 | yl)methyl)pyrrolidine-3-carboxylic acid 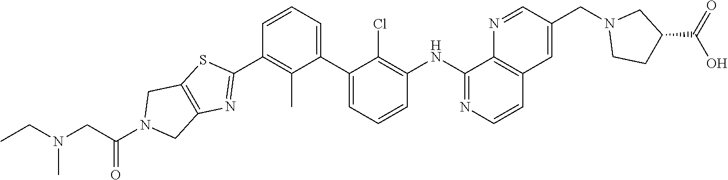 [SMILES: CCN(C)CC(=O)N1Cc2nc(-c3cccc(-c4cccc(Nc5nccc6cc(CN7CC[C@@H](C(=O)O)C7)cnc56)c4Cl)c3C)sc2C1] |
| 14 | US 2018- | (R)-2-(dimethylamino)-1-(2-(3′-(5-(2-(3-hydroxypyrrolidin-1-yl)acetyl)-5,6- |
| | 0177784, | dihydro-4H-pyrrolo[3,4-d]thiazol-2-yl)-2,2′-dimethylbiphenyl-3-yl)-4H- |
| | Example #35 | pyrrolo[3,4-d]thiazol-5(6H)-yl)ethanone 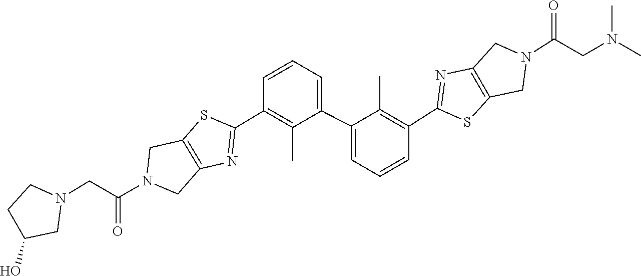 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C] |
| 15 | US 2018- | trans-4-((2-(2′-chloro-3′-(1,5-dimethyl-4,5,6,7-tetrahydro-1H-imidazo[4,5- |
| | 0177870, | c]pyridine-2-carboxamido)-2-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #37 | acid |
| | | 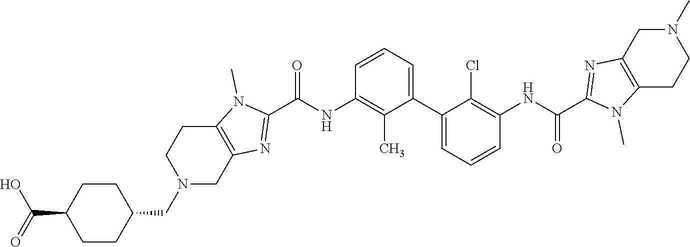 [SMILES: Cc1c(NC(=O)c2nc3c(n2C)CCN(C[C@H]2CC[C@H](C(=O)O)CC2)C3)cccc1-c1cccc(NC(=O)c2nc3c(n2C)CCN(C)C3)c1Cl] |
| 16 | US 2018- | trans-4-(2-(2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)ethyl)cyclohexane-1- |
| | #100 | carboxylic acid |
| | | 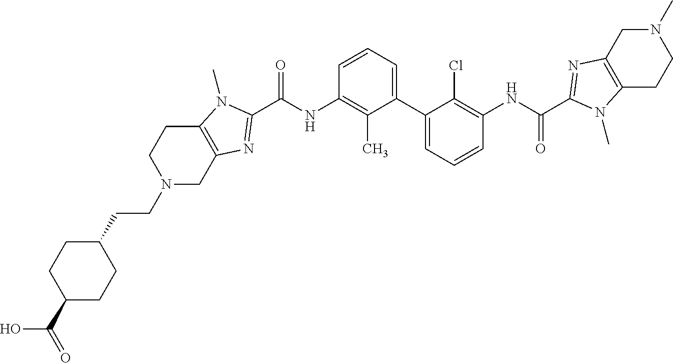 [SMILES: Cc1c(NC(=O)c2nc3c(n2C)CCN(CC[C@H]2CC[C@H](C(=O)O)CC2)C3)cccc1-c1cccc(NC(=O)c2nc3c(n2C)CCN(C)C3)c1Cl] |
| 17 | US 2018- | cis-4-((2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #114 | carboxylic acid |
| | | 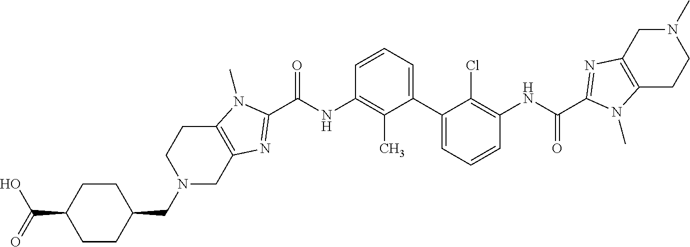 [SMILES: Cc1c(NC(=O)c2nc3c(n2C)CCN(C[C@H]2CC[C@@H](C(=O)O)CC2)C3)cccc1-c1cccc(NC(=O)c2nc3c(n2C)CCN(C)C3)c1Cl] |
| 18 | US 2018- | cis-4-((2-((2-chloro-3′-(1,5-dimethyl-4,5,6,7-tetrahydro-1H-imidazo[4,5- |
| | 0177870, | c]pyridine-2-carboxamido)-2′-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #135 | carboxylic acid |
| | | 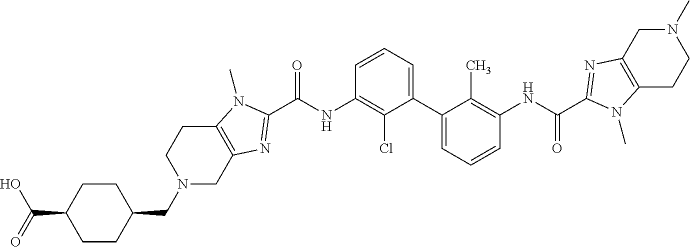 [SMILES: Cc1c(NC(=O)c2nc3c(n2C)CCN(C)C3)cccc1-c1cccc(NC(=O)c2nc3c(n2C)CCN(C[C@H]2CC[C@@H](C(=O)O)CC2)C3)c1Cl] |
| 19 | US 2018- | trans-4-(2-(2-((2′-chloro-2-cyano-3′-(1,5-dimethyl-4,5,6,7-tetrahydro-1H- |
| | 0177870, | imidazo[4,5-c]pyridine-2-carboxamido)-[1,1′-biphenyl]-3-yl)carbamoyl)-1- |
| | Example | methyl-1,4,6,7-tetrahydro-5H-imidazo[4,5-c]pyridin-5-yl)ethyl)cyclohexane-1- |
| | #148 | carboxylic acid |
| | | 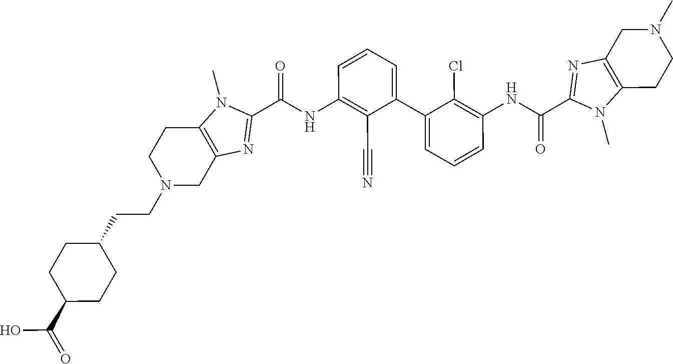 [SMILES: CN1CCc2c(nc(C(=O)Nc3cccc(-c4cccc(NC(=O)c5nc6c(n5C)CCN(CC[C@H]5CC[C@H](C(=O)O)CC5)C6)c4C#N)c3Cl)n2C)C1] |
| 20 | US 2018- | trans-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #159 | acid |
| | | 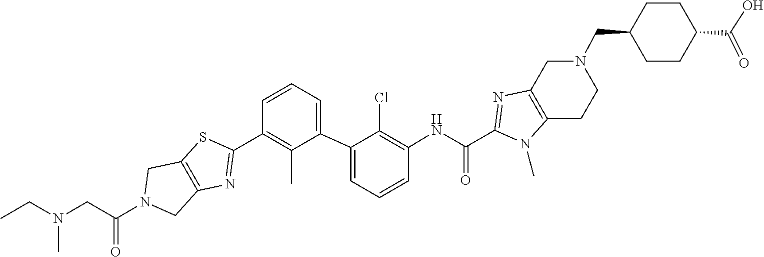 [SMILES: CCN(C)CC(=O)N1Cc2nc(-c3cccc(-c4cccc(NC(=O)c5nc6c(n5C)CCN(C[C@H]5CC[C@H](C(=O)O)CC5)C6)c4Cl)c3C)sc2C1] |
| 21 | US 2018- | cis-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #160 | acid |
| | | 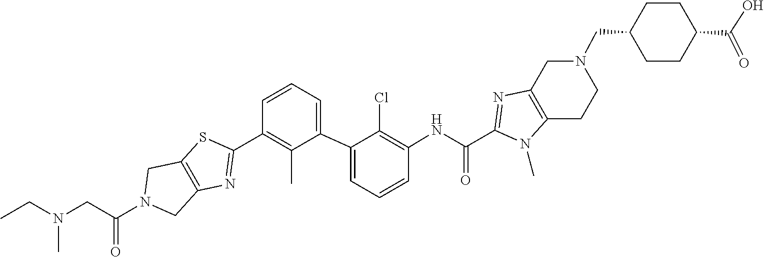 [SMILES: CCN(C)CC(=O)N1Cc2nc(-c3cccc(-c4cccc(NC(=O)c5nc6c(n5C)CCN(C[C@H]5CC[C@@H](C(=O)O)CC5)C6)c4Cl)c3C)sc2C1] |
| 22 | US 2018- | 4-(2-(2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example #161 | dihydro-1H-imidazo[4,5-c]pyridin-5(4H)-yl)ethyl)cyclohexane-1-carboxylic acid 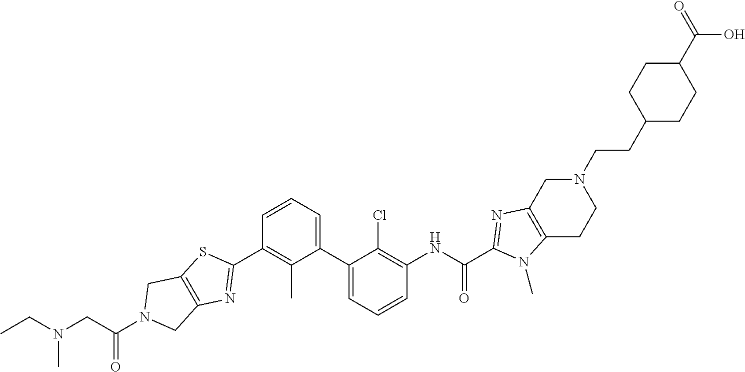 [SMILES: CCN(C)CC(=O)N1Cc2nc(-c3cccc(-c4cccc(NC(=O)c5nc6c(n5C)CCN(CCC5CCC(C(=O)O)CC5)C6)c4Cl)c3C)sc2C1] |
| 23 | US 2018- | 4-(2-(2-(2-chloro-3′-(5-(2-(isopropyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example #162 | dihydro-1H-imidazo[4,5-c]pyridin-5(4H)-yl)ethyl)cyclohexane-1-carboxylic acid 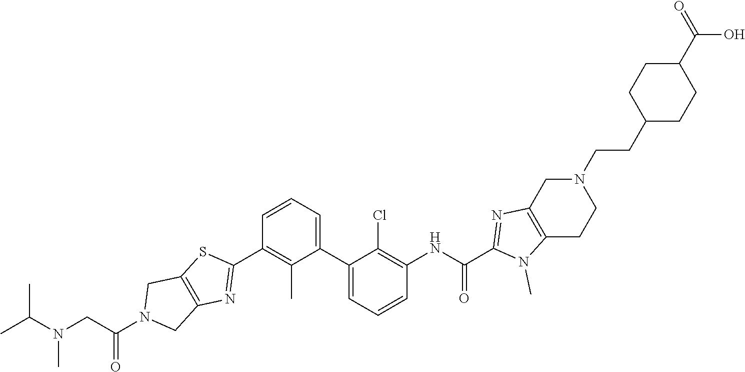 [SMILES: Cc1c(-c2nc3c(s2)CN(C(=O)CN(C)C(C)C)C3)cccc1-c1cccc(NC(=O)c2nc3c(n2C)CCN(CCC2CCC(C(=O)O)CC2)C3)c1Cl] |
| 24 | US 2019- | (R)-1-((7-cyano-2-(3′-(2-(difluoromethyl)-7-((3-hydroxypyrrolidin-1- |
| | 0300524, | yl)methyl)pyrido[3,2-d]pyrimidin-4-ylamino)-2,2′-dimethylbiphenyl-3- |
| | Example #16 | yl)benzo[d]oxazol-5-yl)methyl)piperidine-4-carboxylic acid 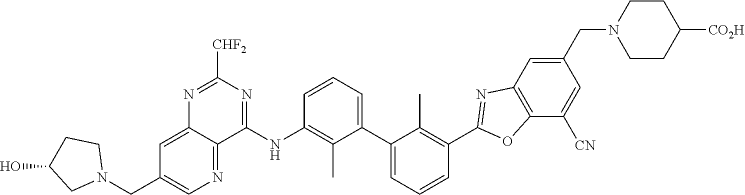 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C] |
| 25 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example #17 | yl)benzo[d]oxazol-5-yl)methyl)pyrrolidine-3-carboxylic acid 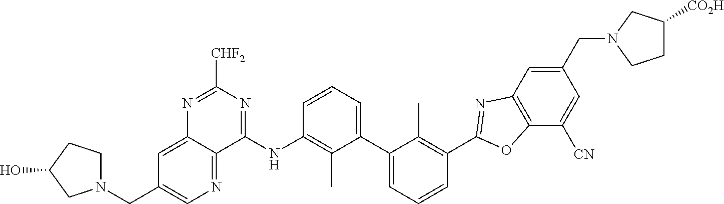 [SMILES: Cc1c(Nc2nc(C(F)F)nc3cc(CN4CC[C@@H](O)C4)cnc23)cccc1-c1cccc(-c2nc3cc(CN4CC[C@@H](C(=O)O)C4)cc(C#N)c3o2)c1C] |
| 26 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example #18 | yl)benzo[d]oxazol-5-yl)methyl)-3-methylpyrrolidine-3-carboxylic acid 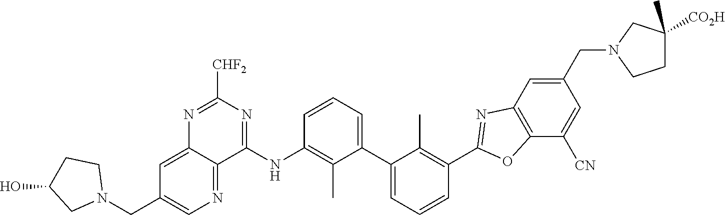 [SMILES: Cc1c(Nc2nc(C(F)F)nc3cc(CN4CC[C@@H](O)C4)cnc23)cccc1-c1cccc(-c2nc3cc(CN4CC[C@@](C)(C(=O)O)C4)cc(C#N)c3o2)c1C] |
| 27 | US 2019- | (R)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example #30 | yl)benzo[d]oxazol-5-yl)methyl)piperidine-4-carboxylic acid 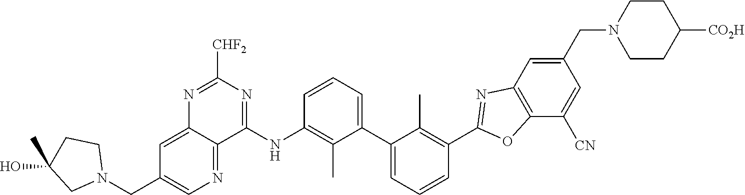 [SMILES: Cc1c(Nc2nc(C(F)F)nc3cc(CN4CC[C@@](C)(O)C4)cnc23)cccc1-c1cccc(-c2nc3cc(CN4CCC(C(=O)O)CC4)cc(C#N)c3o2)c1C] |
| 28 | US 2019- | (S)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example #31 | yl)benzo[d]oxazol-5-yl)methyl)piperidine-4-carboxylic acid 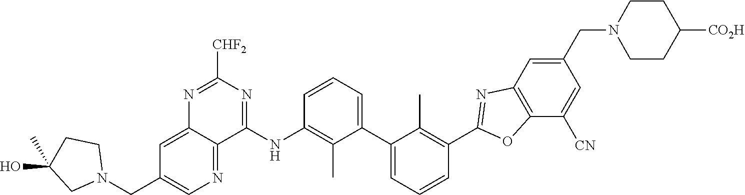 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C] |
| 29 | US 2019- | (R)-4-(2-(2-((2,2′-dichloro-3′-(5-(2-hydroxypropyl)-1-methyl-4,5,6,7-tetrahydro- |
| | 0345170, | 1H-imidazo[4,5-c]pyridine-2-carboxamido)-[1,1′-biphenyl]-3-yl)carbamoyl)-1- |
| | Example | methyl-1,4,6,7-tetrahydro-5H-imidazo[4,5-c]pyridin-5- |
| | #13 | yl)ethyl)bicyclo[2.2.1]heptane-1-carboxylic acid |
| | | 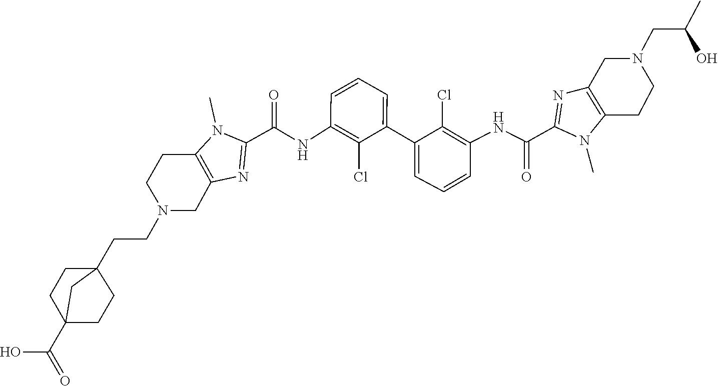 [SMILES: C[C@@H](O)CN1CCc2c(nc(C(=O)Nc3cccc(-c4cccc(NC(=O)c5nc6c(n5C)CCN(CCC57CCC(C(=O)O)(CC5)C7)C6)c4Cl)c3Cl)n2C)C1] |
| 30 | US 2019- | 4,4′-(((((2,2′-dichloro-[1,1′-biphenyl]-3,3′- |
| | 0345170, | diyl)bis(azanediyl)) bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #17 | 1-carboxylic acid) |
| | | 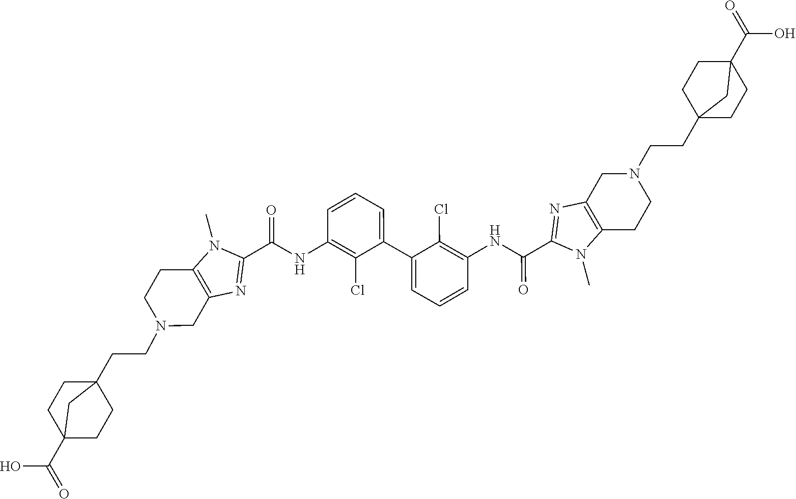 [SMILES: Cn1c(C(=O)Nc2cccc(-c3cccc(NC(=O)c4nc5c(n4C)CCN(CCC46CCC(C(=O)O)(CC4)C6)C5)c3Cl)c2Cl)nc2c1CCN(CCC13CCC(C(=O)O)(CC1)C3)C2] |
| 31 | US 2019- | 4-((2-((3′-(5-(2-(4-carboxybicyclo[2.2.1]heptan-1-yl)ethyl)-1-methyl-4,5,6,7- |
| | 0345170, | tetrahydro-1H-imidazo[4,5-c]pyridine-2-carboxamido)-2,2′-dichloro-[1,1′- |
| | Example | biphenyl]-3-yl)carbamoyl)-1-methyl-1,4,6,7-tetrahydro-5H-imidazo[4,5- |
| | #18 | c]pyridin-5-yl)methyl)bicyclo[2.2.1]heptane-1-carboxylic acid |
| | | 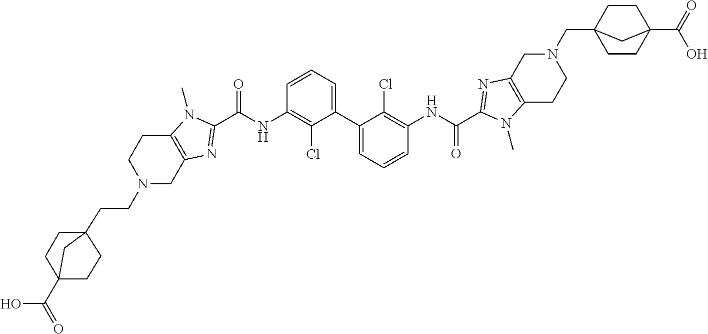 [SMILES: Cn1c(C(=O)Nc2cccc(-c3cccc(NC(=O)c4nc5c(n4C)CCN(CC46CCC(C(=O)O)(CC4)C6)C5)c3Cl)c2Cl)nc2c1CCN(CCC13CCC(C(=O)O)(CC1)C3)C2] |
| 32 | US 2019- | 4,4′-(((((2-chloro-2′-methyl-[1,1′-biphenyl]-3,3′- |
| | 0345170, | diyl)bis(azanediyl))bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #34 | 1-carboxylic acid) |
| | | 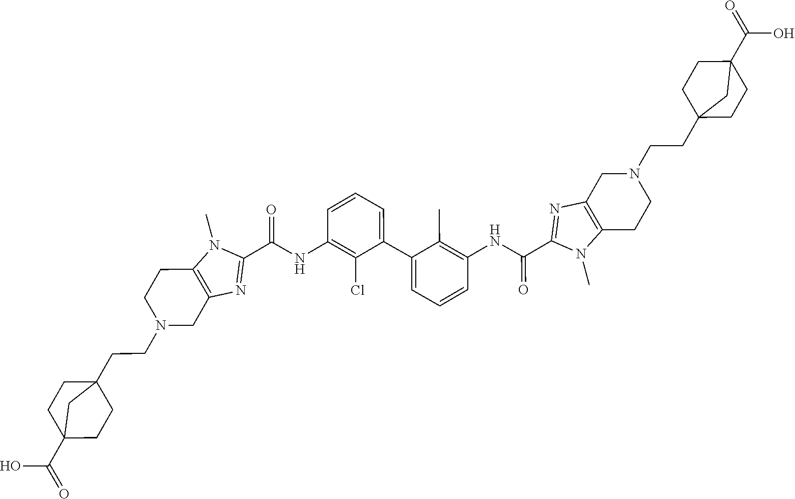 [SMILES: Cc1c(NC(=O)c2nc3c(n2C)CCN(CCC24CCC(C(=O)O)(CC2)C4)C3)cccc1-c1cccc(NC(=O)c2nc3c(n2C)CCN(CCC24CCC(C(=O)O)(CC2)C4)C3)c1Cl] |
| 33 | US 2019- | 4,4′-(((((2-chloro-2′-cyano-[1,1′-biphenyl]-3,3′- |
| | 0345170, | diyl)bis(azanediyl))bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #51 | 1-carboxylic acid) |
| | | 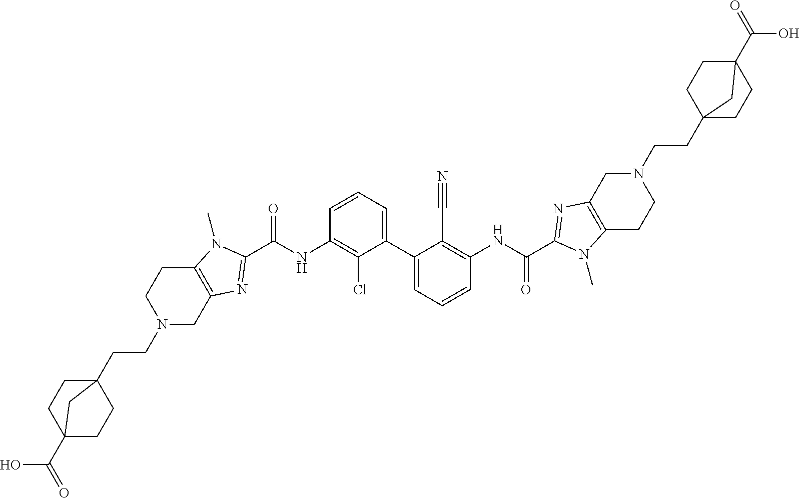 [SMILES: Cn1c(C(=O)Nc2cccc(-c3cccc(NC(=O)c4nc5c(n4C)CCN(CCC46CCC(C(=O)O)(CC4)C6)C5)c3C#N)c2Cl)nc2c1CCN(CCC13CCC(C(=O)O)(CC1)C3)C2] |
| 34 | US 2021- | (R)-4-(2-(2-((2-chloro-3′-((2-(difluoromethyl)-7-((3-hydroxypyrrolidin-1- |
| | 0094976, | yl)methyl)pyrido[3,2-d]pyrimidin-4-yl)amino)-2′-methyl-[1,1′-biphenyl]-3- |
| | Example | yl)carbamoyl)-1-methyl-1,4,6,7-tetrahydro-5H-imidazo[4,5-c]pyridin-5- |
| | #1 | yl)ethyl)bicyclo[2.2.1]heptane-1-carboxylic acid |
| | | 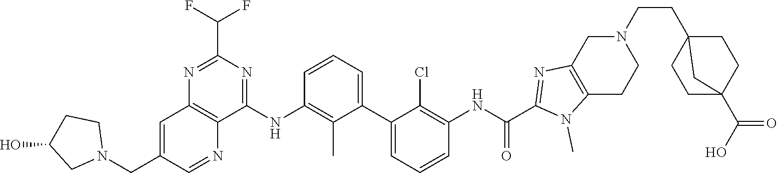 [SMILES: Cc1c(Nc2nc(C(F)F)nc3cc(CN4CC[C@@H](O)C4)cnc23)cccc1-c1cccc(NC(=O)c2nc3c(n2C)CCN(CCC24CCC(C(=O)O)(CC2)C4)C3)c1Cl] |

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1 BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, US 2019/0300524, and US 2019/0345170, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor 20 of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor 10 of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### IV. Formulation, Dosage Forms and Administration

When employed as pharmaceuticals, the compounds of the present disclosure can be administered in the form of pharmaceutical compositions. Thus, the present disclosure provides a composition comprising a compound of Formula (I), a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 g/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

Another aspect of the present invention relates to labeled compounds of the disclosure (radio-labeled, fluorescent-labeled, etc.) that would be useful not only in imaging techniques but also in assays, both in vitro and in vivo, for localizing and quantitating KRAS protein in tissue samples, including human, and for identifying KRAS ligands by inhibition binding of a labeled compound. Substitution of one or more of the atoms of the compounds of the present disclosure can also be useful in generating differentiated ADME (Adsorption, Distribution, Metabolism and Excretion). Accordingly, the present invention includes KRAS binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). A “radio-labelled” compound can refer to an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to other forms of hydrogen.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes, et al., J. Med. Chem. 2011, 54(1), 201-10; R. Xu et al., J. Label. Compd. Radiopharm. 2015, 58, 308-12). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the invention can be used in a screening assay to identify and/or evaluate compounds. For example, a newly synthesized or identified compound (i.e., test compound) which is labeled can be evaluated for its ability to bind a KRAS protein by monitoring its concentration variation when contacting with the KRAS, through tracking of the labeling. For example, a test compound (labeled) can be evaluated for its ability to reduce binding of another compound which is known to bind to a KRAS protein (i.e., standard compound). Accordingly, the ability of a test compound to compete with the standard compound for binding to the KRAS protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of KRAS, such as cancer or infections, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of KRAS according to at least one assay described herein.

### EXAMPLES

Experimental procedures for compounds of the invention are provided below. Preparatory LCMS purifications of some of the compounds prepared were performed on Waters mass directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical liquid chromatography mass spectrometry (LCMS) for purity check.

The compounds separated were typically subjected to analytical LCMS for purity check under the following conditions: Instrument; Agilent 1100 series, LC/MSD, Column: Waters SUNFIRE® C₁₈ 5 μm particle size, 2.1×5.0 mm, Buffers: mobile phase A: 0.025% TFA in water and mobile phase B: MeCN; gradient 2% to 80% of B in 3 min. with flow rate 2.0 mL/min.

Some of the compounds prepared were also separated on a preparative scale by RP-HPLC with MS detector or FCC (silica gel) as indicated in the Examples. Typical prep. RP-HPLC column conditions are as follows:
pH=2 purifications: Waters SUNFIRE® C₁₈ 5 μm particle size, 19×100 mm column, eluting with mobile phase A: 0.1% TFA in water and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with the 30×100 mm column was 60 mL/min.
pH=10 purifications: Waters XBRIDGE® C₁₈ 5 μm particle size, 19×100 mm column, eluting with mobile phase A: 0.15% NH₄OH in water and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with 30×100 mm column was 60 mL/min.

The following abbreviations may be used herein: AcOH (acetic acid); Ac₂O (acetic anhydride); aq. (aqueous); atm. (atmosphere(s));) (2R,3R)-BaryPhos (2-((2R,3R)-3-(tert-butyl)-4-(3,5-dicyclopentyl-2,6-dimethoxyphenyl)-2,3-dihydrobenzo[d][1,3]oxaphosphol-2-yl)propan-2-ol); Boc (t-butoxycarbonyl); Boc₂O (di-t-butyl dicarbonate); BOP ((benzotriazol-1-yloxy)tris(dimethylamino)phosphonium hexafluorophosphate); br (broad); cataCXium® A Pd G3 ([(di(1-adamantyl)-butylphosphine)-2-(2′-amino-1,1′-biphenyl)]palladium(II) methanesulfonate); Cbz (carboxybenzyl); calc. (calculated); CHCl₃ (chloroform); CO (carbon monoxide); CO₂ (carbon dioxide); Cs₂CO₃ (cesium carbonate); CsF (cesium fluoride); d (doublet); dd (doublet of doublets); DBU (1,8-diazabicyclo[5.4.0]undec-7-ene); DCE (1,2-dichloroethane); DCM (dichloromethane); DIAD (N,N′-diisopropyl azidodicarboxylate); DIPEA (N,N-diisopropylethylamine); DIBAL-H (diisobutylaluminium hydride); DMF (N,N-dimethylformamide); DMSO (dimethylsulfoxide); DPEphos (bis[(2-diphenylphosphino)phenyl]ether); Pd(dppf)Cl₂ ([1,1′-Bis(diphenylphosphino)ferrocene]dichloropalladium(II)); Pd(dppf)Cl₂*CH₂Cl₂ ([1,1′-Bis(diphenylphosphino)ferrocene]dichloropalladium(II) complex with dichloromethane); eq. (equivalent(s)); Et (ethyl); EtOH (ethanol); EtOAc (ethyl acetate); Ex. (Example); FCC (flash column chromatography); g (gram(s)); h (hour(s)); H₂ (hydrogen); HATU (N,N,N′,N′tetramethyl-O—(7-azabenzotriazol-1-yl)uronium hexafluorophosphate); HCl (hydrochloric acid or hydrogen chloride); HFIP (1.1.1.3.3.3-hexafluoropropan-2-ol); HPLC (high performance liquid chromatography); Hz (hertz); iPr (isopropyl); iPrMgCl·LiCl (isopropylmagnesium chloride-lithium chloride complex); J (coupling constant); KCN (potassium cyanide); K₂CO₃ (potassium carbonate); KOAc (potassium acetate); KOtBu (potassium tert-butoxide); K₃PO₄ (tribasic potassium phosphate); LCMS (liquid chromatography-mass spectrometry); LiBH₄ (lithium borohydride); LiCl (lithium chloride); LDA (lithium diisopropylamide); LHMDS (lithium bis(trimethylsilyl)amide); m (multiplet); M (molar); mCPBA (3-chloroperoxybenzoic acid); 2-MeTHF (2-methyltetrahydrofuran); MHz (megahertz); MS (Mass spectrometry); Me (methyl); MeCN (acetonitrile); MeOH (methanol); mg (milligram(s)); MgSO₄ (magnesium sulfate); min. (minute(s)); mL (milliliter(s)); mmol (millimole(s)); Ms (methanesulfonyl); N (normal); N₂ (nitrogen); NaBH₄ (sodium borohydride); NaBH₃CN (sodium cyanoborohydride); NaBH(OAc)₃ (sodium triacetoxyborohydride); NaCl (sodium chloride); NaCN (sodium cyanide); Na₂CO₃ (sodium carbonate); NADPH (nicotinamide adenine dinucleotide phosphate); NaH (sodium hydride); NaHCO₃ (sodium bicarbonate); NaHMDS (sodium bis(trimethylsilyl)amide); NaIO₄ (sodium metaperiodate); NaOH (sodium hydroxide); NaOtBu (sodium tert-butoxide); Na₂SO₄ (sodium sulfate); Na₂S₂O₃ (sodium thiosulfate); NBS (N-bromosuccinimide); NCS (N-chlorosuccinimide); NEt₃ (triethylamine); NH₄Cl (ammonium chloride); NH₄OH (ammonium hydroxide); NIS (N-iodosuccinimide); nM (nanomolar); NMP (N-methylpyrrolidinone); NMR (nuclear magnetic resonance spectroscopy); OTf (trifluoromethanesulfonate); Ph (phenyl); Pd₂(dba)₃ (tris(dibenzylideneacetone)dipalladium); Pd₂(dba)₃·CHCl₃ (tris(dibenzylideneacetone)dipalladium-chloroform adduct); Pd(PPh₃)₄ (tetrakis(triphenylphosphine) palladium(0)); pM (picomolar); PPT (precipitate); PPTS (pyridinium p-toluenesulfonate); prep. (preparative); RP-HPLC (reverse phase high performance liquid chromatography); r.t. (room temperature), s (singlet); sat. (saturated); SEM (2-(trimethylsilyl)ethoxymethyl); SEMCl (2-(trimethylsilyl)ethoxymethyl chloride); t (triplet or tertiary); TBDPS (tert-butyldiphenylsilyl); TBS (tert-butyldimethylsilyl); TBSCl (tert-butyldimethylsilyl chloride); tert (tertiary); TsOH (p-toluenesulfonic acid); tt (triplet of triplets); TFA (trifluoroacetic acid); THF (tetrahydrofuran); μg (microgram(s)); μL (microliter(s)); μM (micromolar); wt % (weight percent); xantphos (4,5-bis(diphenylphosphino)-9,9-dimethylxanthene); XPhos Pd G4 ((SP-4-3)-[dicyclohexyl[2′,4′,6′-tris(1-methylethyl)[1,1′-biphenyl]-2-yl]phosphine](methanesulfonato-κO)[2′-(methylamino-κN)[1,1′-biphenyl]-2-yl-KC]palladium); ZnCl₂ (zinc chloride). Brine is sat. aq. NaCl. In vacuo is under vacuum. Other abbreviations used in the literature may also be used.

### Intermediate 1. Methyl 2-amino-4-bromo-6-(((tert-butyldimethylsilyl)oxy)methyl)-5-chloro-3-fluorobenzoate

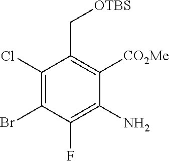 [SMILES: COC(=O)c1c(N)c(F)c(Br)c(Cl)c1CO[Si](C)(C)C(C)(C)C]

### Step 1: Methyl 5-amino-3-bromo-2-chloro-4-fluorobenzoate

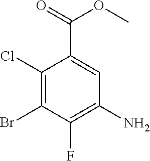

To a solution of methyl 3-amino-5-bromo-4-fluorobenzoate (50.0 g, 202 mmol) in DMF (403 mL) was added NCS (28.3 g, 212 mmol). The resulting mixture was stirred at r.t. overnight then diluted with EtOAc and water. The organic layer was washed with water, brine, dried over Na₂SO₄, filtered and concentrated. The crude material was triturated in hexanes/EtOAc (4:1) and the solid collected by filtration. The filtrate was concentrated to provide an additional batch, which was purified by FCC (eluting with a gradient of 0-30% EtOAc in hexanes) to give two components. The later eluting component from the FCC column is the title compound (total yield: 24.0 g, 42%). LCMS calc. for C₈H₇BrClFNO₂ (M+H)⁺ m/z=281.9, 283.9; found 281.9, 283.9.

### Step 2. (5-Amino-3-bromo-2-chloro-4-fluorophenyl)methanol

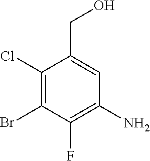 [SMILES: Nc1cc(CO)c(Cl)c(Br)c1F]

LiBH₄ (2.0 M in THF, 302 mL, 603 mmol) was added to a solution of methyl 5-amino-3-bromo-2-chloro-4-fluorobenzoate (42.6 g, 151 mmol) in THF (700 mL) at r.t., followed by MeOH (24.40 mL, 603 mmol). The mixture was stirred at r.t. for 2 h. Sat. aq. NH₄Cl was added and the resulting mixture was extracted with EtOAc. The combined organics were washed with brine, dried over Na₂SO₄ and concentrated. The crude product was used without further purification. LCMS calc. for C₇H₇BrClFNO (M+H)⁺ m/z=253.9, 255.9; found 254.0, 256.0.

### Step 3. (3-Amino-5-bromo-6-chloro-4-fluoro-2-iodophenyl)methanol

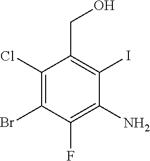 [SMILES: Nc1c(F)c(Br)c(Cl)c(CO)c1I]

To a solution of (5-amino-3-bromo-2-chloro-4-fluorophenyl)methanol (37.3 g, 147 mmol) in EtOH (977 mL) was added silver sulfate (50.3 g, 161 mmol) and iodine (40.9 g, 161 mmol) portionwise, and the resulting mixture was stirred at r.t. overnight. The mixture was poured onto ice-water (1000 mL) and extracted with EtOAc. The organic layer was washed with water (2×), brine, dried over Na₂SO₄, filtered and concentrated. The crude product was purified by FCC (0-30% EtOAc in hexanes) to give the title compound as brown oil which solidified on standing (50.5 g, 91%). LCMS calc. for C₇H₆BrClFINO (M+H)⁺ m/z=379.8, 381.8; found 379.8, 381.8.

### Step 4. 3-Bromo-5-(((tert-butyldimethylsilyl)oxy)methyl)-4-chloro-2-fluoro-6-iodoaniline

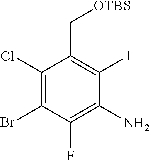 [SMILES: CC(C)(C)[Si](C)(C)OCc1c(Cl)c(Br)c(F)c(N)c1I]

To a solution of (3-amino-5-bromo-6-chloro-4-fluoro-2-iodophenyl)methanol (50.5 g, 133 mmol) and imidazole (11.75 g, 173 mmol) in DMF (266 mL) was added TBSCl (24.0 g, 159 mmol). The solution was stirred at r.t. for 2 h then diluted with sat. aq. NH₄Cl and extracted with EtOAc. The combined organic layers were washed with water and brine, then dried over MgSO₄. After removal of the solvent, the crude product was purified by FCC (eluting with 20% EtOAc in hexanes) to afford the title compound (63.5 g, 97%). LCMS calc. for C₁₃H₂₀BrClFINOSi (M+H)⁺ m/z=493.9, 495.9; found 493.9, 495.9.

### Step 5. Methyl 2-amino-4-bromo-6-(((tert-butyldimethylsilyl)oxy)methyl)-5-chloro-3-fluorobenzoate

A solution of 3-bromo-5-(((tert-butyldimethylsilyl)oxy)methyl)-4-chloro-2-fluoro-6-iodoaniline (63.0 g, 127 mmol), and Pd(dppf)Cl₂ (10.40 g, 12.74 mmol) in toluene (255 mL)/MeOH (255 mL) was deoxygenated for 10 min. with CO gas. Then NEt₃ (53.3 mL, 382 mmol) was added and the solution was stirred at 70° C. under a CO atmosphere for 72 h. The mixture was diluted with sat. aq. NH₄Cl and extracted with EtOAc. The combined organic layers were washed with 1 M HCl and brine, then dried over MgSO₄. After removal of the solvent the crude product was purified by FCC (eluting with 20% EtOAc in hexanes) to afford the title compound (35.7 g, 65.7%). LCMS calc. for C₁₅H₂₃BrClFNO₃Si (M+H)⁺ m/z=426.0, 428.0; found 293.9, 295.9 (M-OTBS)⁺.

### Intermediate 2. 2-(7-Bromo-4,6-dichloro-8-fluoro-2-methylquinolin-5-yl)acetaldehyde

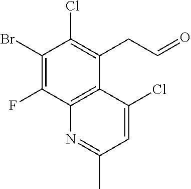 [SMILES: Cc1cc(Cl)c2c(CC=O)c(Cl)c(Br)c(F)c2n1]

### Step 1. Methyl 2-acetamido-4-bromo-6-(((tert-butyldimethylsilyl)oxy)methyl)-5-chloro-3-fluorobenzoate

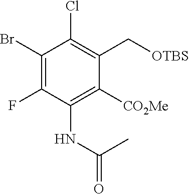 [SMILES: COC(=O)c1c(CO[Si](C)(C)C(C)(C)C)c(Cl)c(Br)c(F)c1NC(C)=O]

Acetyl chloride (4.14 mL, 58.2 mmol) in DCM (100 mL) was added dropwise to a solution of methyl 2-amino-4-bromo-6-(((tert-butyldimethylsilyl)oxy)methyl)-5-chloro-3-fluorobenzoate (Intermediate 1, 22.6 g, 53.0 mmol) and pyridine (5.57 mL, 68.8 mmol) in DCM (350 mL) at r.t. The resulting mixture was stirred at r.t. for 1 h until completion. The mixture was diluted with water and DCM. The organic layer was separated, dried over Na₂SO₄ and concentrated. The crude product was used in the next step without further purification. LCMS calc. for C₁₇H₂₅BrClFNO₄Si (M+H)⁺ m/z=468.0, 470.0; found 468.0, 470.0.

### Step 2. N-(3-Bromo-5-(((tert-butyldimethylsilyl)oxy)methyl)-4-chloro-2-fluoro-6-(hydroxymethyl) phenyl)acetamide

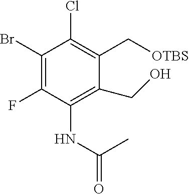 [SMILES: CC(=O)Nc1c(F)c(Br)c(Cl)c(CO[Si](C)(C)C(C)(C)C)c1CO]

LiBH₄ (2.0 M in THF, 79 mL, 159 mmol) was added to a mixture of methyl 2-acetamido-4-bromo-6-(((tert-butyldimethylsilyl)oxy)methyl)-5-chloro-3-fluorobenzoate (24.8 g, 52.9 mmol) in THF (300 mL) at r.t., followed by MeOH (6.42 mL, 159 mmol) dropwise. The mixture was stirred at r.t. for 2 h. Sat. aq. NH₄Cl was added slowly and the resulting mixture was extracted with EtOAc. The combined organics were washed with brine, dried over Na₂SO₄ and concentrated. The crude product was used in the next step without further purification (19.1 g, 82% over 2 steps). LCMS calc. for C₁₆H₂₅BrClFNO₃Si (M+H)⁺ m/z=440.0, 442.0; found 440.0, 442.0.

### Step 3. N-(3-Bromo-5-(((tert-butyldimethylsilyl)oxy)methyl)-4-chloro-2-fluoro-6-formylphenyl)acetamide

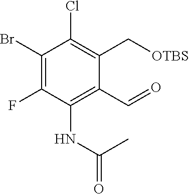 [SMILES: CC(=O)Nc1c(F)c(Br)c(Cl)c(CO[Si](C)(C)C(C)(C)C)c1C=O]

To a solution of N-(3-bromo-5-(((tert-butyldimethylsilyl)oxy)methyl)-4-chloro-2-fluoro-6-(hydroxymethyl)phenyl)acetamide (13.0 g, 29.5 mmol) in DCM (300 mL) was added Dess-Martin periodinane (13.13 g, 31.0 mmol). The resulting mixture was stirred for 15 min., then quenched with sat. aq. NaHCO₃ and stirred for 30 min. The organic layer was separated, dried over Na₂SO₄ and concentrated. The residue was purified by FCC (eluting with a gradient of 0-30% EtOAc in hexanes) to give the title compound (6.8 g, 53%). LCMS calc. for C₁₆H₂₃BrClFNO₃Si (M+H)⁺ m/z=438.0, 440.0; found 438.0, 440.0.

### Step 4. N-(3-Bromo-5-(((tert-butyldimethylsilyl)oxy)methyl)-4-chloro-2-fluoro-6-(1-hydroxyethyl)phenyl)acetamide

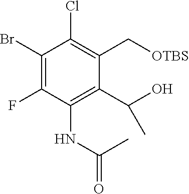 [SMILES: CC(=O)Nc1c(F)c(Br)c(Cl)c(CO[Si](C)(C)C(C)(C)C)c1C(C)O]

To solution of N-(3-bromo-5-(((tert-butyldimethylsilyl)oxy)methyl)-4-chloro-2-fluoro-6-formylphenyl)acetamide (1.52 g, 3.46 mmol) in THF (35 mL) was added methylmagnesium bromide (3.0 M in diethyl ether, 2.54 mL, 7.62 mmol) at 0° C. After stirring for 1 h at 0° C., the reaction mixture was diluted with water and EtOAc. The organic layer was separated, washed with brine, dried with Na₂SO₄ and concentrated. The crude product was used in the next step without further purification. LCMS calc. for C₁₇H₂₇BrClFNO₃Si (M+H)⁺ m/z=454.0, 456.0; found 454.0, 456.0.

### Step 5. N-(2-Acetyl-5-bromo-3-(((tert-butyldimethylsilyl)oxy)methyl)-4-chloro-6-fluorophenyl)acetamide

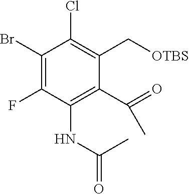 [SMILES: CC(=O)Nc1c(F)c(Br)c(Cl)c(CO[Si](C)(C)C(C)(C)C)c1C(C)=O]

To a solution of N-(2-acetyl-5-bromo-3-(((tert-butyldimethylsilyl)oxy)methyl)-4-chloro-6-fluorophenyl)acetamide (1.58 g, 3.49 mmol) in DCM (35 mL) was added Dess-Martin periodinane (1.55 g, 3.66 mmol). The resulting mixture was stirred for 1 h 15 min., then quenched with sat. aq. NaHCO₃ and stirred for 30 min. The organic layer was separated and dried over Na₂SO₄ and concentrated. The residue was triturated with EtOAc/hexanes (1:10) to give the title compound as white solid. An additional portion of product was obtained by purifying the mother liquor by FCC (eluting with a gradient of 0-30% EtOAc in hexanes) to give the title compound (combined yield 1.18 g, 75%). LCMS calc. for C₁₇H₂₅BrClFNO₃Si (M+H)⁺ m/z=452.0, 454.0; found 452.0, 454.0.

### Step 6. 7-Bromo-6-chloro-8-fluoro-5-(hydroxymethyl)-2-methylquinolin-4-ol

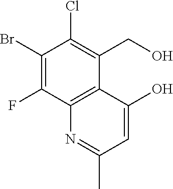 [SMILES: Cc1cc(O)c2c(CO)c(Cl)c(Br)c(F)c2n1]

N-(2-Acetyl-5-bromo-3-(((tert-butyldimethylsilyl)oxy)methyl)-4-chloro-6-fluorophenyl)acetamide (1.18 g, 2.61 mmol) was suspended in tert-butanol (26.1 mL). KOtBu (1.0 M in THF, 5.73 mL, 5.73 mmol) was added. The reaction mixture was stirred at 80° C. for 4 h. After cooling to r.t., the reaction was quenched by the addition of 3 M HCl to adjust the pH to about 5. The slurry was stirred for 10 min. at r.t. 10 mL of MeCN was added, followed by water (5 mL) then ether (20 mL). The mixture was stirred for another 30 min., and then the solids were collected by filtration and washed with ether and hexane. The wet cake was dried under vacuum to give the title compound (0.30 g, 36%). LCMS calc. for C₁₁H₉BrClFNO₂ (M+H)⁺ m/z=319.9, 321.9; found 319.9, 321.9.

### Step 7. 7-Bromo-4,6-dichloro-5-(chloromethyl)-8-fluoro-2-methylquinoline

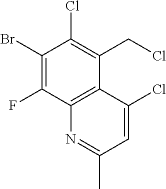 [SMILES: Cc1cc(Cl)c2c(CCl)c(Cl)c(Br)c(F)c2n1]

7-Bromo-6-chloro-8-fluoro-5-(hydroxymethyl)-2-methylquinolin-4-ol (288 mg, 0.898 mmol) was dissolved in POCl₃ (1675 μL, 17.97 mmol). The resulting mixture was stirred at 100° C. for 1.5 h.

POCl₃ was removed by azeotrope with toluene (3×), and the residue was diluted with DCM and sat. aq. NaHCO₃. The organic layer was separated and dried over Na₂SO₄ and concentrated. The crude product was purified with FCC (eluting with a gradient of 0-20% EtOAc in hexanes) to yield the title compound as a white solid (275 mg, 86%). LCMS calc. for C₁₁H₇BrCl₃FN (M+H)⁺ m/z=355.9, 357.9, 359.9; found 355.9, 357.9, 359.9.

### Step 8. 2-(7-Bromo-4,6-dichloro-8-fluoro-2-methylquinolin-5-yl)MeCN

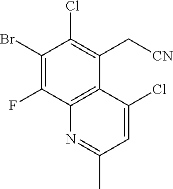 [SMILES: Cc1cc(Cl)c2c(CC#N)c(Cl)c(Br)c(F)c2n1]

To a solution of 7-bromo-4,6-dichloro-5-(chloromethyl)-8-fluoro-2-methylquinoline (507 mg, 1.418 mmol) in DMF (7 mL) was added NaCN (83 mg, 1.7 mmol). The reaction mixture was stirred at r.t. for 3 h. The reaction mixture was diluted with EtOAc and water. The organic layer was separated, washed with water, brine, dried over Na₂SO₄ and concentrated. The crude product was purified with FCC (eluting with a gradient of 0-30% EtOAc in hexanes) to give the title compound as a white solid (394 mg, 80%). LCMS calc. for C₁₂H₇BrCl₂FN₂ (M+H)⁺ m/z=346.9, 348.9; found 346.9, 348.9.

### Step 9. 2-(7-Bromo-4,6-dichloro-8-fluoro-2-methylquinolin-5-yl)acetaldehyde

To a solution of 2-(7-bromo-4,6-dichloro-8-fluoro-2-methylquinolin-5-yl)MeCN (394 mg, 1.132 mmol) in DCM (11 mL) at −78° C. was added DIBAL-H (1.0 M in DCM, 1.7 mL, 1.7 mmol). The resulting mixture was stirred at −78° C. for 40 min. then warmed to −10° C. for 1.5 h. The reaction was quenched with MeOH. Sat. aq. Rochelle salt was added to the solution at ≤10° C. The biphasic mixture was stirred vigorously for 1 h at r.t. and separated. The organic layer was washed with brine (×2), then dried over Na₂SO₄ and concentrated. The residue was purified by FCC (eluting with a gradient of 0-40% EtOAc in hexanes) to give the title compound (336 mg, 85%). LCMS calc. for C₁₂H₈BrCl₂FNO (M+H)⁺ m/z=349.9, 351.9; found 349.9, 351.9.

### Intermediate 3. 2-(7-bromo-2,4,6-trichloro-8-fluoroquinolin-5-yl)acetonitrile

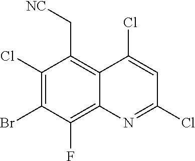 [SMILES: N#CCc1c(Cl)c(Br)c(F)c2nc(Cl)cc(Cl)c12]

### Step 1. 3-Bromo-2-chloro-4-fluoro-1-methyl-5-nitrobenzene

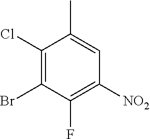

To a stirred solution of 2-bromo-3-fluoro-6-methyl-4-nitroaniline (24 g, 96 mmol) in MeCN (482 mL) was added copper(I) chloride (19.08 g, 193 mmol) followed by tert-butylnitrite (22.92 mL, 193 mmol) with stirring at r.t. The mixture was stirred at 70° C. for 1 h. The reaction mixture was concentrated, filtered over diatomaceous earth and the filter cake was washed with EtOAc. The filtrate was concentrated and used directly for the next step without further purification. LCMS calc. for C₇H₅BrClFNO₂ (M+H)⁺ m/z=267.9, 269.9; found 267.9, 269.9.

### Step 2. 3-Bromo-4-chloro-2-fluoro-5-methylaniline

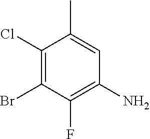

3-Bromo-2-chloro-4-fluoro-1-methyl-5-nitrobenzene (537 mg, 2 mmol), iron (1.1 g, 20.00 mmol), and NH₄Cl (1117 mg, 20.88 mmol) were dissolved in EtOH (3.3 mL)/THF (3.3 mL)/water (3.3 mL). The mixture was then heated and stirred for 1 h at 60° C. under an atmosphere of N₂. The mixture was diluted with EtOAc (10 mL) and water (20 mL), and then filtered through diatomaceous earth which was then washed with MeOH. Two layers formed. The mixture was transferred to a separatory funnel, diluted with brine (10 mL) and extracted with EtOAc (15 mL) (3×). The organic layers were washed with sat. aq. NaCl, dried over MgSO₄, and concentrated. The crude product was purified by FCC (0-20% heptane/EtOAc) to provide the title compound. LCMS calc. for C₇H₇BrClFN (M+H)⁺ m/z=237.9, 239.9; found 237.9, 239.9.

### Step 3. Ethyl 3-((3-bromo-4-chloro-2-fluoro-5-methylphenyl)amino)-3-oxopropanoate

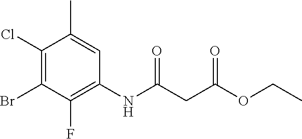 [SMILES: CCOC(=O)CC(=O)Nc1cc(C)c(Cl)c(Br)c1F]

3-Bromo-4-chloro-2-fluoro-5-methylaniline (5 g, 20.97 mmol) was dissolved in EtOAc (70 mL). Sat. aq. NaHCO₃ was added (70 mL) and the mixture was stirred rapidly. Ethyl malonyl chloride (3.36 mL, 26.2 mmol) was added dropwise and stirring at r.t. was continued for 20 min. at r.t. Upon completion the organic layer was separated and the aqueous layer was extracted with EtOAc (2×). The combined organic phases were washed with brine, dried over MgSO₄ and concentrated. The crude product was used in the next step without further purification. LCMS calc. for C₁₂H₁₃BrClFNO₃ (M+H)⁺ m/z=352.0, 354.0; found 352.0, 354.0.

### Step 4. 3-((3-Bromo-4-chloro-2-fluoro-5-methylphenyl)amino)-3-oxopropanoic acid

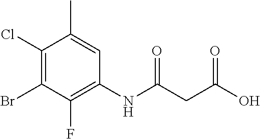 [SMILES: Cc1cc(NC(=O)CC(=O)O)c(F)c(Br)c1Cl]

Ethyl 3-((3-bromo-4-chloro-2-fluoro-5-methylphenyl)amino)-3-oxopropanoate (7.4 g, 21.02 mmol) was dissolved in THF (25 mL). EtOH (25 mL) was added to the reaction mixture followed by aq. KOH (25.2 mL, 2.5 M, 63.1 mmol) at r.t. The reaction mixture was stirred rapidly for 90 min. The mixture was poured into a mixture of 2 M HCl (100 mL) and ice (50 g), resulting in precipitation. The solid was collected by filtration and washed with water, then air dried. The filtrate was extracted with 1 M NaOH, then acidified with 1 M HCl to pH˜7. The mixture was then extracted with EtOAc, dried over Na₂SO₄ and concentrated to provide additional material. The crude product was used in the next step without further purification (6.3 g, 92%). LCMS calc. for C₁₀H₉BrClFNO₃ (M+H)⁺ m/z=323.9, 325.9; found 323.9, 325.9.

### Step 5. 7-Bromo-6-chloro-8-fluoro-5-methylquinoline-2,4-diol

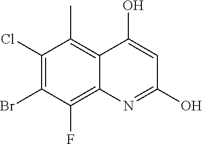 [SMILES: Cc1c(Cl)c(Br)c(F)c2nc(O)cc(O)c12]

3-((3-Bromo-4-chloro-2-fluoro-5-methylphenyl)amino)-3-oxopropanoic acid (5.65 g, 17.41 mmol) and phosphorus pentoxide (7.7 wt. %) in methanesulfonic acid (33.2 mL, 209 mmol) were mixed and stirred under an N₂ atmosphere. The mixture was stirred at 85° C. for 3 h. The mixture was then allowed to cool to r.t., then quenched into ice with rapid stirring. The resulting precipitate was collected by filtration, washed with cold water, then air dried to provide the title compound, which was used in the next step without further purification (4.07 g, 13.28 mmol, 76%). LCMS calc. for C₁₀H₇BrClFNO₂ (M+H)⁺ m/z=305.9, 307.9; found 305.9, 307.9.

### Step 6. 7-Bromo-2,4,6-trichloro-8-fluoro-5-methylquinoline

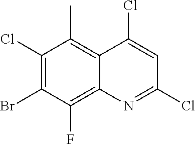 [SMILES: Cc1c(Cl)c(Br)c(F)c2nc(Cl)cc(Cl)c12]

7-Bromo-6-chloro-8-fluoro-5-methylquinoline-2,4 (1H,3H)-dione (3.07 g, 10.02 mmol), POCl₃ (9.34 mL, 100 mmol), and DIPEA (5.25 mL, 30.0 mmol) were dissolved in toluene (17 mL) under N₂. The reaction mixture was heated at 100° C. for 1 h, then concentrated. The remaining POCl₃ was slowly quenched with aq. NaHCO₃, then the mixture was extracted with DCM. The organic layer was dried over MgSO₄ and concentrated. The crude product was purified by FCC (0-30% EtOAc/heptane) to provide the title compound (2.67 g, 78%). LCMS calc. for C₁₀H₅BrCl₃FN (M+H)⁺ m/z=341.9, 343.9, 345.9; found 341.9, 343.9, 345.9.

### Step 7. 7-Bromo-5-(bromomethyl)-2,4,6-trichloro-8-fluoroquinoline

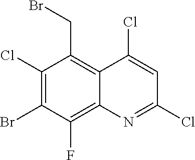 [SMILES: Fc1c(Br)c(Cl)c(CBr)c2c(Cl)cc(Cl)nc12]

7-Bromo-2,4,6-trichloro-8-fluoro-5-methylquinoline (23.4 mg, 0.068 mmol), NBS (20.21 mg, 0.112 mmol), and AIBN (3.36 mg, 0.020 mmol) were dissolved in trifluorotoluene (700 μL). The reaction mixture was placed under a N₂ atmosphere, then stirred at 80° C. under N₂ for 15 min. The mixture was diluted with DCM, washed with water and brine, dried over MgSO₄ and concentrated. The crude product was purified by FCC (0-30% EtOAc/heptane) to provide the title compound (19 mg, 66.0%) as a white solid. LCMS calc. for C₁₀H₄Br₂Cl₃FN (M+H)⁺ m/z=421.8, 423.8; found 421.8, 423.8.

### Step 8. 2-(7-Bromo-2,4,6-trichloro-8-fluoroquinolin-5-yl)acetonitrile

7-Bromo-5-(bromomethyl)-2,4,6-trichloro-8-fluoroquinoline (800 mg, 1.894 mmol) was dissolved in DCM (10 mL). The mixture was cooled to 0° C. with rapid stirring, then tetrabutylammonium cyanide (51 mg, 0.189 mmol) followed by potassium cyanide (370 mg, 5.68 mmol) were added to the reaction mixture as solids. The mixture was then allowed to warm to r.t. and stirred for 30 min. The mixture was then diluted with DCM and the layers were separated. The aqueous layer was extracted with DCM and the combined organic layers were washed with sat. aq. NaHCO₃ and brine, dried over MgSO₄ and concentrated. The crude product was purified by FCC (0-100 heptane/MTBE) to provide the title compound (563 mg, 81%) as a white solid. LCMS calc. for C₁₁H₄BrCl₃FN₂ (M+H)⁺ m/z=366.8, 368.8, 370.8; found 366.8, 368.8, 370.8.

### Intermediate 4. 2-(7-bromo-2,6-dichloro-8-fluoro-4-(methylthio)quinolin-5-yl)acetaldehyde

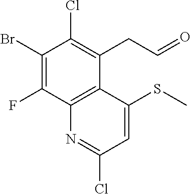 [SMILES: CSc1cc(Cl)nc2c(F)c(Br)c(Cl)c(CC=O)c12]

### Step 1. 2-(7-Bromo-2,6-dichloro-8-fluoro-4-(methylthio)quinolin-5-yl)acetonitrile

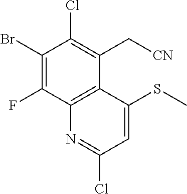 [SMILES: CSc1cc(Cl)nc2c(F)c(Br)c(Cl)c(CC#N)c12]

Intermediate 3 (4.95 g, 13.44 mmol) was dissolved in MeCN (134 mL) under N₂. NaSMe (6.48 mL, 20.15 mmol) was added to the reaction mixture as an aqueous solution. The mixture was stirred at r.t., with additional aq. NaSMe was added as necessary to push the reaction to full conversion. When the reaction was complete as judged by LCMS, the mixture was diluted with water and sat. aq. NH₄Cl and extracted with a mixture of EtOAc and DCM. The combined organics were dried over MgSO₄ and concentrated. The crude product was used in the next step without further purification. LCMS calc. for C₁₂H₇BrCl₂FN₂S (M+H)⁺: m/z=378.9, 380.9; found 378.9, 380.9.

### Step 2. 2-(7-Bromo-2,6-dichloro-8-fluoro-4-(methylthio)quinolin-5-yl)acetaldehyde

2-(7-Bromo-2,6-dichloro-8-fluoro-4-(methylthio)quinolin-5-yl)acetonitrile (5.0 g, 13.16 mmol) was dissolved in toluene (132 mL) under N₂. The mixture was stirred until dissolution was complete and then cooled to −78° C. DIBAL-H (1 M in DCM, 19.73 mL, 19.73 mmol) was added to the reaction mixture slowly. The mixture was stirred for 1 h at −78° C. At this time, ethyl formate (10.71 mL, 132 mmol) was dissolved in 1 mL of THF and the total solution was added slowly to the reaction mixture. Then, MeOH (5.32 mL, 132 mmol) was dissolved in THF (1 mL) and this solution was added to the reaction mixture. Rochelle salt was added and the mixture was diluted with DCM. The mixture was stirred rapidly and the layers were separated. The aqueous layer was extracted with DCM. The combined organics were dried over MgSO₄ and concentrated. The residue was triturated with MTBE to provide the title compound, which was of suitable quality to be used in the next step without further purification (4.1 g, 83%). LCMS calc. for C₁₂H₃BrCl₂FNOS (M+H)⁺: m/z=381.9, 383.9; found 381.9, 383.9.

### Intermediate 5. (E)-7-Bromo-4,6-dichloro-8-fluoro-2-methyl-5-(2-(4,4,5,5-tetramethyl-1,3,2-dioxaborolan-2-yl)vinyl)quinoline

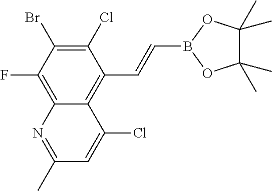 [SMILES: Cc1cc(Cl)c2c(/C=C/B3OC(C)(C)C(C)(C)O3)c(Cl)c(Br)c(F)c2n1]

### Step 1. 2-Amino-4-bromo-5-chloro-3-fluorobenzoic acid

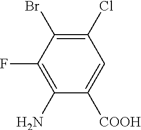 [SMILES: Nc1c(C(=O)O)cc(Cl)c(Br)c1F]

NCS (34.5 g, 258 mmol) was added to a stirred solution of 2-amino-4-bromo-3-fluorobenzoic acid (50.4 g, 215 mmol) in DMF (360 mL). The mixture was heated at 80° C. for 3 h. After cooling to ambient temperature, the mixture was poured into 400 mL water/ice mixture with stirring, resulting in precipitation. The solids were collected on a fritted filter, 10 washed with minimal EtOAc and hexanes and dried under vacuum to afford the title compound (46.8 g, 81%). LCMS calc. for C₇H₅BrClFNO₂ (M+H)⁺: m/z=267.9, 269.9; found 267.9, 269.9.

### Step 2. 7-Bromo-6-chloro-8-fluoro-2H-benzo[d][1,3]oxazine-2,4 (1H)-dione

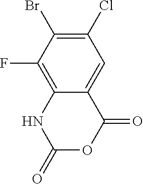 [SMILES: O=c1[nH]c2c(F)c(Br)c(Cl)cc2c(=O)o1]

To a stirred solution containing 2-amino-4-bromo-5-chloro-3-fluorobenzoic acid (46.8 g, 174 mmol) in THF (580 mL), triphosgene (34.7 g, 117 mmol) was added. The mixture was heated at 60° C. for 3 h. After cooling to r.t., the mixture was concentrated. The resulting solids were washed with hexanes and dried under reduced vacuum to afford the title compound (41.5 g, 81%). The title compound does not readily ionize under LCMS conditions.

### Step 3. Ethyl 7-bromo-6-chloro-8-fluoro-4-hydroxy-2-methylquinoline-3-carboxylate

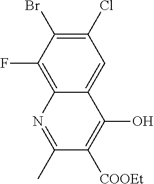 [SMILES: CCOC(=O)c1c(C)nc2c(F)c(Br)c(Cl)cc2c1O]

To a stirred solution containing 7-bromo-6-chloro-8-fluoro-2H-benzo[d][1,3]oxazine-2,4 (1H)-dione (27.3 g, 93 mmol) in DMSO (93 mL) was added ethyl acetoacetate sodium salt (15.5 g, 102 mmol). The mixture was heated at 80° C. for 1 h. After cooling to r.t., the mixture was poured into aq. HCl (1 M) with stirring resulting in precipitation. The solids were collected on a fritted filter, washed with minimal EtOAc and hexanes and dried under vacuum to afford the title compound (29.8 g, 89%). LCMS calc. for C₁₃H₁₁BrClFNO₃ (M+H)⁺: m/z=362.0, 364.0; found 362.0, 364.0.

### Step 4. 7-Bromo-6-chloro-8-fluoro-2-methylquinolin-4-ol

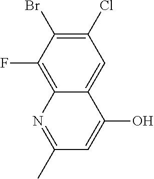

To a flask containing ethyl 7-bromo-6-chloro-8-fluoro-4-hydroxy-2-methylquinoline-3-carboxylate (29.8 g, 82 mmol) was added diphenyl ether (70 mL). The mixture was heated at 250° C. for 0.5 h. After cooling to r.t., hexanes was added to the mixture with stirring resulting in precipitation. The tan solids were collected on a fritted filter and dried under vacuum to afford the title compound (14.9 g, 63%). LCMS calc. for C₁₀H₇BrClFNO (M+H)⁺: m/z=289.9, 291.9; found 289.9, 291.9.

### Step 5. 7-Bromo-4,6-dichloro-8-fluoro-2-methyl-5-((triisopropylsilyl)ethynyl)quinoline

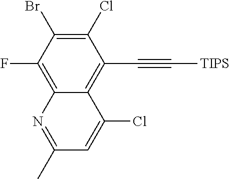

A solution containing 7-bromo-6-chloro-8-fluoro-2-methylquinolin-4-ol (10.0 g, 34.4 mmol), KOAc (6.76 g, 68.6 mmol), dichloro(p-cymene)ruthenium(II) dimer (5.27 g, 8.61 mmol) and (2-bromoethynyl)tris(propan-2-yl)silane (8.83 mL, 36.1 mmol) in 1,4-dioxane (86 mL) was heated at 110° C. under N₂ atmosphere for 2 h. After cooling to r.t., the reaction mixture was poured into aq. HCl (1 M, 100 mL) with stirring. The resulting mixture was extracted using EtOAc (3×). The organic layers were combined, dried over Na₂SO₄ and concentrated. POCl₃ (60 mL) was added and the mixture was heated at 100° C. for 2 h. After cooling to r.t., the volatiles were removed under reduced pressure. After azeotroping with toluene (3×), the residue was dissolved in DCM and treated with sat. aq. NaHCO₃. The mixture was extracted with DCM, dried over Na₂SO₄, filtered and concentrated. The crude product was purified by FCC (eluting with DCM) to provide the title compound (9.5 g, 56%). LCMS calc. for C₂₁H₂₆BrCl₂FNSi (M+H)⁺: m/z=488.0, 490.0; found 488.0, 490.0.

### Step 6. (E)-7-Bromo-4,6-dichloro-8-fluoro-2-methyl-5-(2-(4,4,5,5-tetramethyl-1,3,2-dioxaborolan-2-yl)vinyl)quinoline

To a solution containing 7-bromo-4,6-dichloro-8-fluoro-2-methyl-5-((triisopropylsilyl)ethynyl)quinoline (3.86 g, 7.89 mmol) in THF (40 mL) was added tetrabutylammonium fluoride (1 M in THF, 11.8 mL, 11.8 mmol). After stirring at r.t. for 0.5 h, the mixture was diluted with EtOAc. The organic extract was washed with brine and then filtered through silica padded on a fritted filter. The silica was washed with EtOAc. The combined filtrate was dried over Na₂SO₄, filtered and concentrated to afford a solid.

In a flask containing copper(I) chloride (0.252 g, 2.55 mmol), DPEphos (1.373 g, 2.55 mmol) and NaOtBu (0.490 g, 5.10 mmol) was added THF (8.5 mL). After stirring for 0.5 h under N₂ atmosphere, a THF (8.5 mL) solution containing bis(pinacolate)diboron (2.59 g, 10.2 mmol) was added to the mixture. The resulting mixture was stirred at r.t. for another 10 min. Then, the solid prepared as described in the preceding paragraph was added in one portion followed by MeOH (0.7 mL, 17 mmol). The resulting mixture was stirred at r.t. for 16 h. Upon completion, the mixture was diluted with EtOAc, washed with brine, dried over Na₂SO₄, filtered and concentrated under reduced pressure. The crude product was purified by FCC (0-40% EtOAc/hexanes) to provide the title compound (2.5 g, 64%). LCMS calc. for C₁₃H₁₉BBrCl₂FNO₂ (M+H)⁺: m/z=460.0, 462.0; found 460.0, 462.0.

### Intermediate 6. 2-(7-Bromo-6-chloro-8-fluoro-2-iodo-4-(methylthio)quinolin-5-yl)acetaldehyde

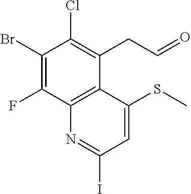 [SMILES: CSc1cc(I)nc2c(F)c(Br)c(Cl)c(CC=O)c12]

### Step 1. 2-(7-Bromo-6-chloro-8-fluoro-2-iodo-4-(methylthio)quinolin-5-yl)acetonitrile

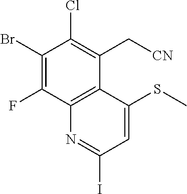 [SMILES: CSc1cc(I)nc2c(F)c(Br)c(Cl)c(CC#N)c12]

To a solution of 2-(7-bromo-2,6-dichloro-8-fluoro-4-(methylthio)quinolin-5-yl)acetonitrile (Intermediate 4, Step 1, 9.0 g, 23.7 mmol) in MeCN (500 mL) were added NaI (35.5 g, 237 mmol) and TMSCl (6.0 mL, 47.4 mmol). The mixture was stirred at 60° C. overnight, then poured into rapidly stirring cold water (1 L). The mixture was filtered, then extracted with DCM. The organic layer was dried over Na₂SO₄ and concentrated, then purified by FCC (0-100% DCM in heptane) to provide the title compound (10.5 g, 94%). LCMS calc. for C₁₂H₇BrClFIN₂S (M+H)⁺ m/z=470.8, 472.8; found 470.8, 472.8.

### Step 2. 2-(7-Bromo-6-chloro-8-fluoro-2-iodo-4-(methylthio)quinolin-5-yl)acetaldehyde

To a solution of 2-(7-bromo-6-chloro-8-fluoro-2-iodo-4-(methylthio)quinolin-5-yl)acetonitrile (3.97 g, 8.42 mmol) in DCM (120 mL) at −78° C. was added DIBAL-H (1 M in DCM, 12.6 mL, 12.6 mmol) and the mixture was stirred at −78° C. for 90 min., then quenched with ethyl formate (6.85 mL, 84 mmol) and MeOH (3.4 mL, 84 mmol). Sat. aq. Rochelle's salt was then added and the resulting mixture was stirred vigorously at r.t. for 1 h. The mixture was extracted with DCM. The organic layer was dried over MgSO₄ and concentrated. The crude product was used in the next step without further purification (4.0 g, >99%). LCMS calc. for C₁₂H₉BrClFIN₂S (M+H)⁺ m/z=472.8, 474.8; found 472.8, 474.8.

### Intermediate 7. 2-(7-Bromo-4-chloro-8-fluoro-6-iodo-2-methylquinolin-5-yl)acetaldehyde

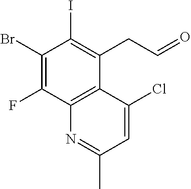 [SMILES: Cc1cc(Cl)c2c(CC=O)c(I)c(Br)c(F)c2n1]

### Step 1. 3-Bromo-2-fluoro-4-iodoaniline

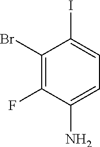

To a solution of 3-bromo-2-fluoroaniline (31 g, 163 mmol) in DMF (163 mL) was added NIS (38.5 g, 171 mmol) and the reaction mixture was stirred at r.t. for 3 h, then poured into water. The mixture was extracted with EtOAc. The organic layer was washed with water and brine, dried over Na₂SO₄ and concentrated. The crude product was dissolved in MeOH and HCl (4.0 M in 1,4-dioxane, 40 mL, 160 mmol) was added causing the precipitation of a solid. This solid was collected by filtration and washed with cold MeOH to provide a white solid, which was used in the next step without further purification (36.4 g, 63% yield, HCl salt assumed). LCMS calc. for C₆H₅BrFIN (M+H)⁺ m/z=315.9, 317.9; found 315.9, 317.9.

### Step 2. 7-Bromo-8-fluoro-6-iodo-2-methylquinolin-4-ol

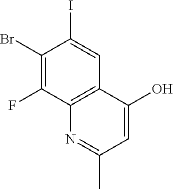

A mixture of 3-bromo-2-fluoro-4-iodoaniline, HCl salt (15 g, 42.6 mmol), ethyl acetoacetate (11.1 g, 85 mmol) and polyphosphoric acid (61 g, 255 mmol) was heated at 150° C. for 2 h, then poured into ice water. The mixture was stirred vigorously at r.t. and the resulting solid was collected by filtration. The off-white solid was triturated with THF/hexanes to provide the title compound as a white solid (10.6 g, 65%). LCMS calc. for C₁₀H₇BrFINO (M+H)⁺ m/z=381.9, 383.9; found 381.9, 383.9.

### Step 3. 7-Bromo-8-fluoro-6-iodo-2-methyl-5-((triisopropylsilyl)ethynyl)quinolin-4-ol

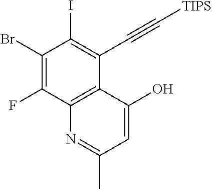

To a solution of 7-bromo-8-fluoro-6-iodo-2-methylquinolin-4-ol (5.2 g, 13.6 mmol), KOAc and [RuCl₂(p-cymene)]₂ (2.08 g, 0.25 mmol) in 1,4-dioxane (97 mL) was added (bromoethynyl)triisopropylsilane (6.0 mL, 24.5 mmol) and the reaction mixture was placed under air and heated at 110° C. overnight. Aq. HCl (1 M) was added and the mixture was extracted with EtOAc. The organic layer was dried over Na₂SO₄ and concentrated. The crude product was purified by FCC (0-100% EtOAc in hexanes) to provide the title compound as a white solid (4.38 g, 57%). LCMS calc. for C₂₁H₂₇BrFINOSi (M+H)⁺ m/z=562.0, 564.0; found 562.0, 564.0.

### Step 4. 7-Bromo-4-chloro-8-fluoro-6-iodo-2-methyl-5-((triisopropylsilyl)ethynyl)quinoline

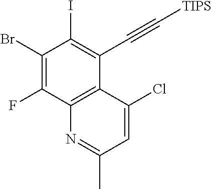

A solution of 7-bromo-8-fluoro-6-iodo-2-methyl-5-((triisopropylsilyl)ethynyl)quinolin-4-ol (4.38 g, 7.8 mmol) in POCl₃ (14.5 mL, 156 mmol) was heated at 100° C. for 1 h, then concentrated and azeotroped with toluene. The residue was quenched with a mixture of sat. aq. NaHCO₃ and DCM. The layers were separated and the aqueous layer was extracted with DCM. The combined organic phases were dried over Na₂SO₄ and concentrated. The crude product was purified by FCC (eluting with DCM) to provide the title compound (3.82 g, 84%). LCMS calc. for C₂₁H₂₆BrClFINSi (M+H)⁺ m/z=580.0, 582.0; found 580.0, 582.0.

### Step 5. 7-Bromo-4-chloro-5-ethynyl-8-fluoro-6-iodo-2-methylquinoline

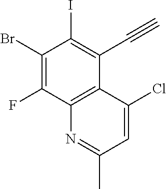 [SMILES: C#Cc1c(I)c(Br)c(F)c2nc(C)cc(Cl)c12]

To a solution of 7-bromo-4-chloro-8-fluoro-6-iodo-2-methyl-5-((triisopropylsilyl)ethynyl)quinoline (2.0 g, 3.44 mmol) in THF (11.5 mL) was added TBAF (1.0 M in THF, 4.1 mL, 4.1 mmol) and the reaction mixture was stirred at r.t. for 30 min., then quenched with water and extracted with EtOAc. The organic layer was washed with brine, dried over Na₂SO₄ and concentrated. The crude product was used in the next step without further purification (1.39 g, 95%). LCMS calc. for C₁₂H₆BrClFIN (M+H)⁺ m/z=423.8, 425.8; found 423.8, 425.8.

### Step 6. (E)-7-Bromo-4-chloro-8-fluoro-6-iodo-2-methyl-5-(2-(4,4,5,5-tetramethyl-1,3,2-dioxaborolan-2-yl)vinyl)quinoline

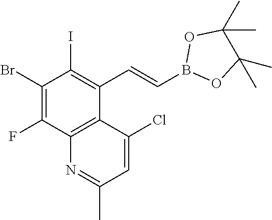 [SMILES: Cc1cc(Cl)c2c(/C=C/B3OC(C)(C)C(C)(C)O3)c(I)c(Br)c(F)c2n1]

To a solution of 7-bromo-4-chloro-5-ethynyl-8-fluoro-6-iodo-2-methylquinoline (1.53 g, 3.6 mmol) in toluene (18 mL) was added (PPh₃)₃RuCl(CO)H (0.68 g, 0.72 mmol) and the reaction flask was evacuated and back filled with N₂. HBpin (1.0 mL, 7.19 mmol) was then added and the reaction mixture was stirred at 60° C. for 2 h. The reaction was quenched with water and extracted with EtOAc. The organic layer was washed with brine, dried over Na₂SO₄ and concentrated. The crude product was purified by FCC (0-60% EtOAc in hexanes). The residue was triturated with hexanes to provide an orange solid (1.04 g, 52%). LCMS calc. for C₁₃H₁₉BBrClFINO₂ (M+H)⁺ m/z=551.9, 553.9; found 551.9, 553.9.

### Step 7. 2-(7-Bromo-4-chloro-8-fluoro-6-iodo-2-methylquinolin-5-yl)acetaldehyde

A solution of alanine methyl ester, HCl salt (0.27 g, 1.95 mmol) and DIPEA (0.34 mL, 1.95 mmol) was stirred in DCM at r.t. until the solution was clear. To this mixture were added PPTS (0.33 g, 1.3 mmol), (E)-7-bromo-4-chloro-8-fluoro-6-iodo-2-methyl-5-(2-(4,4,5,5-tetramethyl-1,3,2-dioxaborolan-2-yl)vinyl)quinoline (718 mg, 1.3 mmol), sodium borate tetrahydrate (0.40 g, 2.60 mmol) and water (1 V). The reaction mixture was stirred at r.t. for 2 h, then quenched with water and extracted with DCM. The organic layer was dried over Na₂SO₄ and concentrated. The crude product was purified by FCC (0-100% EtOAc in hexanes) to provide the title compound. LCMS calc. for C₁₂H₃BrClFINO (M+H)⁺ m/z=441.8, 443.8; found 441.8, 443.8.

### Intermediate 8. 2-(7-Bromo-6-chloro-8-fluoro-2-methyl-4-(methylthio)quinolin-5-yl)acetaldehyde

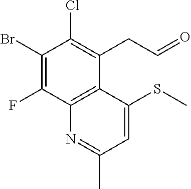 [SMILES: CSc1cc(C)nc2c(F)c(Br)c(Cl)c(CC=O)c12]

### Step 1. 2-(7-Bromo-6-chloro-8-fluoro-2-methyl-4-(methylthio)quinolin-5-yl)acetonitrile

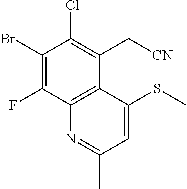 [SMILES: CSc1cc(C)nc2c(F)c(Br)c(Cl)c(CC#N)c12]

To a solution of 2-(7-bromo-6-chloro-8-fluoro-2-iodo-4-(methylthio)quinolin-5-yl)acetonitrile (Intermediate 6, Step 1, 10.1 g, 21.4 mmol) in 1,4-dioxane (214 mL) were added methylboronic acid (6.4 g, 107 mmol), Cs₂CO₃ (14 g, 43 mmol) and (dppf)PdCl₂ (1.75 g, 2.14 mmol). The reaction was placed under a N₂ atmosphere, then heated at 80° C. for 2 h. The mixture was diluted with water and EtOAc and the layers were separated. The aqueous layer was extracted with EtOAc and the organic layer were washed with water and brine, dried over Na₂SO₄ and concentrated. The crude product was purified by FCC (0-70% EtOAc in hexanes) to provide the title compound (4.05 g, 53%). LCMS calc. for C₁₃H₁₀BrClFN₂S (M+H)⁺ m/z=359.0, 361.0; found 359.0, 361.0.

### Step 2. 2-(7-Bromo-6-chloro-8-fluoro-2-methyl-4-(methylthio)quinolin-5-yl)acetaldehyde

To a solution of 2-(7-bromo-6-chloro-8-fluoro-2-methyl-4-(methylthio)quinolin-5-yl)acetonitrile (2.0 g, 5.56 mmol) in DCM (70 mL) at −78° C. was added DIBAL-H (1.0 M in DCM, 8.3 mL, 8.3 mmol) dropwise over 5 min. The reaction mixture was stirred at −78° C. for 1 h, then quenched with MeOH (2.25 mL, 55.6 mmol) dropwise. Rochelle's salt was then added and the mixture was warmed to r.t. and stirred vigorously for 1 h. The mixture was extracted with DCM. The organic layer was dried over Na₂SO₄ and concentrated. The crude product was used in the next step without further purification. LCMS calc. for C₁₃H₁₁BrClFNOS (M+H)⁺ m/z=362.0, 364.0; found 362.0, 364.0.

### Intermediate 9. tert-Butyl (4-(6-chloro-8-fluoro-2-methyl-4-(methylthio)-5-(2-oxoethyl)quinolin-7-yl)-3-cyano-7-fluorobenzo[b]thiophen-2-yl)carbamate

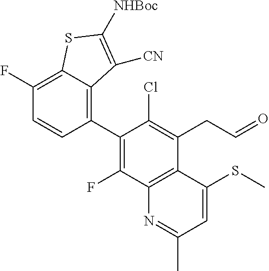 [SMILES: CSc1cc(C)nc2c(F)c(-c3ccc(F)c4sc(NC(=O)OC(C)(C)C)c(C#N)c34)c(Cl)c(CC=O)c12]

### Step 1. 7-Bromo-6-chloro-5-(2,2-dimethoxyethyl)-8-fluoro-2-methyl-4-(methylthio)quinoline

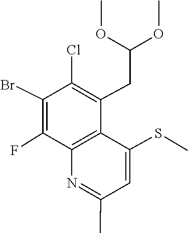 [SMILES: COC(Cc1c(Cl)c(Br)c(F)c2nc(C)cc(SC)c12)OC]

To a solution of Intermediate 8 (839 mg, 1.40 mmol) and TsOH (44 mg, 0.23 mmol) in MeOH (23 mL) was added trimethyl orthoformate (5.0 mL, 46 mmol) and the reaction mixture was heated at 60° C. and stirred overnight. After cooling to r.t., the reaction was quenched with sat. aq. NaHCO₃ then concentrated. The residue was extracted with EtOAc. The organic layer was washed with brine, dried over Na₂SO₄ and concentrated. The crude product was used in the next step without further purification (579 mg, 61%). LCMS calc. for C₁₅H₁₇BrClFNO₂S (M+H)⁺ m/z=408.0, 410.0; found 408.0, 410.0.

### Step 2. tert-Butyl (4-(6-chloro-5-(2,2-dimethoxyethyl)-8-fluoro-2-methyl-4-(methylthio)quinolin-7-yl)-3-cyano-7-fluorobenzo[b]thiophen-2-yl)carbamate

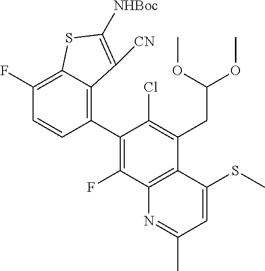 [SMILES: COC(Cc1c(Cl)c(-c2ccc(F)c3sc(NC(=O)OC(C)(C)C)c(C#N)c23)c(F)c2nc(C)cc(SC)c12)OC]

To a mixture of 7-bromo-6-chloro-5-(2,2-dimethoxyethyl)-8-fluoro-2-methyl-4-(methylthio)quinoline (580 mg, 1.42 mmol), Cs₂CO₃ (1.40 g, 4.26 mmol), tert-butyl (3-cyano-4-(5,5-dimethyl-1,3,2-dioxaborinan-2-yl)-7-fluorobenzo[b]thiophen-2-yl)carbamate (1.15 g, 2.84 mmol) and dichloro[bis(2-(diphenylphosphino)phenyl)ether]palladium(II) (254 mg, 0.36 mmol) was added 1,4-dioxane (12 mL) and the reaction flask was evacuated, back filled with N₂, then stirred at 100° C. for 1 h. The reaction was cooled to r.t., then diluted with EtOAc and filtered through a plug of diatomaceous earth. The filtrate was concentrated, then purified by FCC (0-100% EtOAc in hexanes) to provide the title compound (696 mg, 79%). LCMS calc. for C₂₉H₂₉ClF₂N₃O₄S₂(M+H)⁺ m/z=620.1; found 620.1.

### Step 3. tert-Butyl (4-(6-chloro-8-fluoro-2-methyl-4-(methylthio)-5-(2-oxoethyl)quinolin-7-yl)-3-cyano-7-fluorobenzo[b]thiophen-2-yl)carbamate

To a solution of tert-butyl (4-(6-chloro-5-(2,2-dimethoxyethyl)-8-fluoro-2-methyl-4-(methylthio)quinolin-7-yl)-3-cyano-7-fluorobenzo[b]thiophen-2-yl)carbamate (593 mg, 0.96 mmol) in DCM (12 mL)/acetone (3 mL) was added iron chloride hexahydrate (570 mg, 2.1 mmol) and the reaction mixture was stirred at r.t. 2 h, then quenched with sat. aq. NaHCO₃. The mixture was extracted with EtOAc and the organic layer was washed with brine, dried over Na₂SO₄ and concentrated. The crude product was purified by FCC (0-100% EtOAc in hexanes) to provide the title compound (410 mg, 75%). LCMS calc. for C₂₇H₂₃ClF₂N₃O₃S₂(M+H)⁺ m/z=574.1; found 574.1.

### Intermediate 10. (R)-3-(8-Bromo-7-chloro-9-fluoro-2-iodo-5,6-dihydro-4H-benzo[de][1,6]naphthyridin-4-yl)-1-methylpyrrolidin-2-one

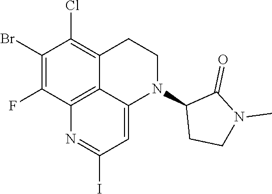 [SMILES: CN1CC[C@@H](N2CCc3c(Cl)c(Br)c(F)c4nc(I)cc2c34)C1=O]

(R)-3-Amino-1-methylpyrrolidin-2-one 4-methylbenzenesulfonate salt (2.85 g, 10 mmol) was dissolved in THF (40 mL), DCM (40 mL), and HFIP (13 mL). DIPEA (4.0 mL, 22.65 mmol) was then added and the mixture was stirred at r.t. for 5 min., followed by the addition of Intermediate 6 (4.3 g, 9.1 mmol), NaBH(OAc)₃ (3.84 g, 18.12 mmol) and AcOH (5.6 mL, 72.5 mmol). The resulting mixture was stirred at r.t. for 3 h. The reaction was quenched with aq. NaHCO₃ and the mixture was extracted with EtOAc. The organic layer was washed with water and brine, dried over Na₂SO₄ and concentrated. The crude was dissolved in DMSO (20 mL) and stirred at 90° C. for 1.5 h. The reaction was quenched with water and extracted with EtOAc. The organic layer was washed with water and brine, dried over Na₂SO₄ and concentrated. The crude product was purified by FCC (0-100% EtOAc in Hexane) to provide the title compound (3.3 g, 69%). LCMS calc. for C₁₆H₁₄BrClFIN₃O (M+H)⁺ m/z=523.9, 525.9; found 523.9, 525.9.

### Intermediate 11. (3R,5S)-3-amino-1-(methyl-d₃)-5-(trifluoromethyl)pyrrolidin-2-one

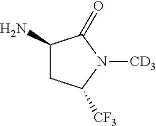 [SMILES: [2H]C([2H])([2H])N1C(=O)[C@H](N)C[C@H]1C(F)(F)F]

### Step 1. (S)-1-(Methyl-d₃)-5-(trifluoromethyl)pyrrolidin-2-one

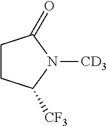 [SMILES: [2H]C([2H])([2H])N1C(=O)CC[C@H]1C(F)(F)F]

To a mixture of (S)-5-(trifluoromethyl)pyrrolidin-2-one (16.3 g, 106 mmol) and Cs₂CO₃ (69.4 g, 213 mmol) in MeCN (355 mL), was added iodomethane-d₃ (9.28 mL, 149 mmol) at r.t. and the reaction mixture was heated at 70° C. for 5.5 h. The mixture was diluted with DCM and filtered through a plug of diatomaceous earth. The frit was washed with additional DCM. The filtrate was filtered once again, then concentrated. The crude product was of sufficient purity to be used in the next step (16.7 g, 92%). LCMS calc. for C₆H₆D₃F₃NO (M+H)⁺ m/z=171.1; found 171.1.

### Step 2. (3R,5S)-3-Azido-1-(methyl-d3)-5-(trifluoromethyl)pyrrolidin-2-one

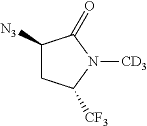 [SMILES: [2H]C([2H])([2H])N1C(=O)[C@H](N=[N+]=[N-])C[C@H]1C(F)(F)F]

To a solution of (S)-1-(methyl-d₃)-5-(trifluoromethyl)pyrrolidin-2-one (6.48 g, 38.1 mmol) in THF (190 mL) at −78° C. was added LDA (1.0 M in THF/hexanes, 40.0 mL, 40.0 mmol) and the mixture was stirred at −78° C. for 35 min. A solution of 2,4,6-triisopropylbenzenesulfonyl azide (12.37 g, 40.0 mmol) in toluene (100 mL) was then added dropwise over 30 min. while maintaining the reaction temperature at −78° C. The reaction mixture was then warmed to −60° C. and stirred at this temperature for 3 h. The reaction was quenched with AcOH (4.4 mL, 76 mmol) and allowed to warm to r.t. The mixture was diluted with sat. aq. ammonium chloride and extracted with EtOAc. The organic layer was washed with brine, dried over Na₂SO₄ and concentrated. The crude product was purified by FCC (0-3% EtOAc in DCM) to provide the title compound (9.0 g, 87% purity, 98%) as a single diastereomer. LCMS calc. for C₆H₅D₃F₃N₄O (M+H)⁺ m/z=212.1; found 212.1.

### Step 3. (3R,5S)-3-Amino-1-(methyl-d₃)-5-(trifluoromethyl)pyrrolidin-2-one

To a solution of (3R,5S)-3-azido-1-(methyl-d₃)-5-(trifluoromethyl)pyrrolidin-2-one (9.0 g, 87.5% purity, 37.2 mmol) in EtOAc (186 mL) was added palladium on carbon (10% by weight, 1.98 g, 1.86 mmol) and the reaction flask was evacuated, back filled with hydrogen gas from a balloon, then stirred under a hydrogen atmosphere overnight. The mixture was filtered through a plug of diatomaceous earth and the filtrate concentrated. The crude product was used in the next step without further purification (7.7 g, 85% purity, 96%). LCMS calc. for C₆H₇D₃F₃N₂O (M+H)⁺ m/z=186.1; found 186.1.

### Intermediate 12. (3R,5S)-3-(8-Bromo-7-chloro-9-fluoro-2-methyl-5,6-dihydro-4H-benzo[de][1,6]naphthyridin-4-yl)-1-(methyl-d₃)-5-(trifluoromethyl)pyrrolidin-2-one

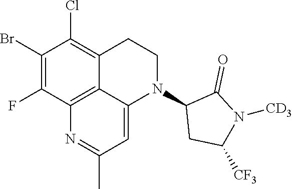 [SMILES: [2H]C([2H])([2H])N1C(=O)[C@H](N2CCc3c(Cl)c(Br)c(F)c4nc(C)cc2c34)C[C@H]1C(F)(F)F]

Intermediate 11 (0.312 g, 2.66 mmol) was dissolved in DCM/THF/HFIP (3:3:1, 10 mL total). Intermediate 8 (510 mg, 1.41 mmol), NaBH(OAc)₃ (1.12 g, 5.63 mmol) and AcOH (0.64 mL, 11.3 mmol) were then added, the resulting mixture was stirred at r.t. overnight. The reaction was quenched with aq. NaHCO₃ and extracted with EtOAc. The organic layer was washed with brine, dried over Na₂SO₄ and concentrated. The crude product was purified by FCC (0-10% MeOH in DCM) to provide the title compound (650 mg, 96%). LCMS calc. for C₁₃H₁₃D₃BrClF₄N₃O (M+H)⁺ m/z=483.0, 485.0; found 483.0, 485.0.

### Intermediate 13. (3R,5S)-3-(8-Bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one

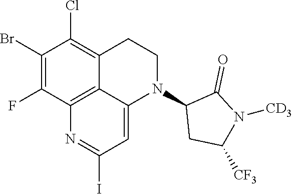 [SMILES: [2H]C([2H])([2H])N1C(=O)[C@H](N2CCc3c(Cl)c(Br)c(F)c4nc(I)cc2c34)C[C@H]1C(F)(F)F]

The title compound was prepared according to the general procedure described for Intermediate 10, using Intermediate 11 as the coupling partner. LCMS calc. for C₁₇H₁₀D₃BrClF₄IN₃O (M+H)⁺ m/z=594.9, 596.9; found 594.9, 596.9.

### Intermediate 14. (3R,5S)-3-(7-chloro-9-fluoro-2-methyl-8-(tributylstannyl)-5,6-dihydro-4H-benzo[de][1,6]naphthyridin-4-yl)-1-(methyl-d₃)-5-(trifluoromethyl)pyrrolidin-2-one

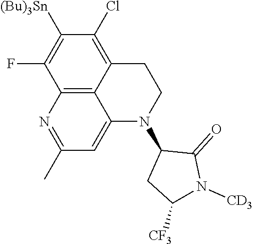

Under N₂, a solution of (3R,5S)-3-(8-bromo-7-chloro-9-fluoro-2-methyl-5,6-dihydro-4H-benzo[de][1,6]naphthyridin-4-yl)-1-(methyl-d₃)-5-(trifluoromethyl)pyrrolidin-2-one (Intermediate 12, 100 mg, 0.207 mmol), hexabutylditin (240 mg, 0.413 mmol), tris(dibenzylideneacetone)dipalladium (18.9 mg, 0.21 mmol), tricyclohexyl phosphine (11.6 mg, 0.041 mmol) and LiCl (26.3 mg, 0.620 mmol) in 1,4-dioxane (2.0 mL) was stirred at 110° C. for 5 h. After completion, the mixture was concentrated under vacuum. The residue was purified by FCC (0-70% EtOAc in hexanes) to provide the title compound (63 mg, 44%). LCMS calc. for C₃₀H₄₀D₃ClF₄N₃OSn (M+H)⁺ m/z=695.2; found 695.2.

### Intermediate 15. (3R,5S)-3-(8-bromo-7-chloro-9-fluoro-2-(hydroxymethyl)-5,6-dihydro-4H-benzo[de][1,6]naphthyridin-4-yl)-1-(methyl-d₃)-5-(trifluoromethyl)pyrrolidin-2-one

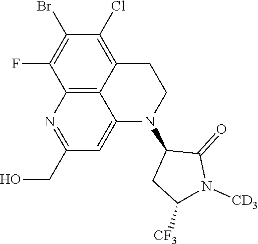 [SMILES: [2H]C([2H])([2H])N1C(=O)[C@H](N2CCc3c(Cl)c(Br)c(F)c4nc(CO)cc2c34)C[C@H]1C(F)(F)F]

### Step 1. (3R,5S)-3-(8-Bromo-7-chloro-9-fluoro-2-vinyl-5,6-dihydro-4H-benzo[de][1,6]naphthyridin-4-yl)-1-(methyl-d₃)-5-(trifluoromethyl)pyrrolidin-2-one

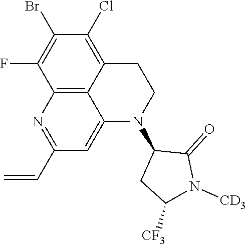 [SMILES: [2H]C([2H])([2H])N1C(=O)[C@H](N2CCc3c(Cl)c(Br)c(F)c4nc(C=C)cc2c34)C[C@H]1C(F)(F)F]

To the mixture of (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 500 mg, 0.840 mmol), [1,1′ bis(diphenylphosphino)ferrocene]dichloropalladium(II) (123 mg, 0.168 mmol), Na₂CO₃ (222 mg, 2.10 mmol), and 2-vinylboronic acid pinacolester (749 μL, 4.20 mmol) was added 1,4-dioxane (3498 μL)/water (700 μL) under N₂. The mixture heated at 60° C. for 1.5 h before cooling to r.t., then diluting with EtOAc and aq. NaHCO₃. The layers were separated, and the organic layer was washed with brine, dried over Na₂SO₄ and concentrated. The crude product was purified by FCC (10-80% EtOAc in hexanes) to provide the title compound (357 mg, 86%). LCMS calc. for C₁₉H₁₃D₃BrClF₄N₃O (M+H)⁺ m/z=495.0, 497.0; found 495.0, 497.0.

### Step 2. 8-Bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbaldehyde

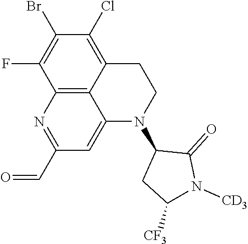 [SMILES: [2H]C([2H])([2H])N1C(=O)[C@H](N2CCc3c(Cl)c(Br)c(F)c4nc(C=O)cc2c34)C[C@H]1C(F)(F)F]

To a THF (3.5 mL)/water (0.5 mL) solution of (3R,5S)-3-(8-bromo-7-chloro-9-fluoro-2-vinyl-5,6-dihydro-4H-benzo[de][1,6]naphthyridin-4-yl)-1-(methyl-d₃)-5-(trifluoromethyl)pyrrolidin-2-one (247 mg, 0.498 mmol) was added OsO₄ solution (4 wt % in water, 391 μL, 0.062 mmol) at r.t. The mixture was stirred for 3 min. before NaIO₄ (266 mg, 1.25 mmol) was added, the resulting mixture was stirred at r.t. for 2 h. The mixture was then diluted with DCM, the layers were separated, and the organic layer was washed with brine, dried over MgSO₄ and concentrated. The crude product was purified by FCC (0-100% EtOAc in heptane, then 0-100% acetone in heptane) to provide the title compound (140.4 mg, 57%). LCMS calc. for C₁₃H₁₁D₃BrClF₄N₃O₂ (M+H)⁺ m/z=497.0, 499.0; found 497.0, 499.0.

### Step 3. (3R,5S)-3-(8-Bromo-7-chloro-9-fluoro-2-(hydroxymethyl)-5,6-dihydro-4H-benzo[de][1,6]naphthyridin-4-yl)-1-(methyl-d₃)-5-(trifluoromethyl)pyrrolidin-2-one

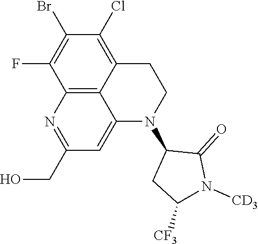 [SMILES: [2H]C([2H])([2H])N1C(=O)[C@H](N2CCc3c(Cl)c(Br)c(F)c4nc(CO)cc2c34)C[C@H]1C(F)(F)F]

To a 0° C. solution of 8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbaldehyde (140 mg, 0.281 mmol) in 2-Me-THF (1875 μL)/MeOH (938 μL) was added NaBH₄ (8.0 mg, 0.21 mmol). The mixture was stirred for 5 min before quenching with sat. aq. NH₄Cl, and diluting with EtOAc, and separating, and the organic layer was then washed with brine, dried over MgSO₄, and concentrated to provide the title compound (131.4 mg, 93%). LCMS calc. for C₁₃H₁₃D₃BrClF₄N₃O₂ (M+H)⁺ m/z=499.0, 501.0; found 499.0, 501.0.

### Example 1a and 1b. 2-Amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

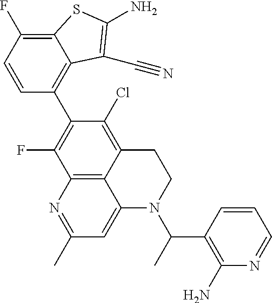 [SMILES: Cc1cc2c3c(c(Cl)c(-c4ccc(F)c5sc(N)c(C#N)c45)c(F)c3n1)CCN2C(C)c1cccnc1N]

### Step 1. 3-(1-(8-Bromo-7-chloro-9-fluoro-2-methyl-5,6-dihydro-4H-benzo[de][1,6]naphthyridin-4-yl)ethyl)-N,N-bis(4-methoxybenzyl)pyridin-2-amine

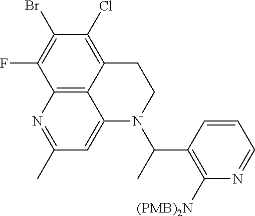

3-(1-Aminoethyl)-N,N-bis(4-methoxybenzyl)pyridin-2-amine (397 mg, 1.053 mmol) was added to a solution of 2-(7-bromo-4,6-dichloro-8-fluoro-2-methylquinolin-5-yl)acetaldehyde (336 mg, 0.957 mmol) in THF (4.8 mL). The mixture was stirred at r.t. for 30 min. Separately, a mixture of NaBH(OAc)₃ (812 mg, 3.83 mmol), DCM (4.8 mL) and TFA (295 μL, 3.83 mmol) was stirred for 30 min. The two solutions were then combined. The resulting mixture was stirred at r.t. for 3 h. The reaction mixture was diluted with sat. aq. NaHCO₃ and DCM. The aqueous layer was extracted with DCM. The combined organic layers were dried over Na₂SO₄ and concentrated. The crude product was used in the next step without further purification (300 mg, 46%). LCMS calc. for C₃₅H₃₄BrClFN₄O₂(M+H)⁺ m/z=675.1, 677.1; found 675.1, 677.1.

### Step 2. 3-(1-(8-Bromo-7-chloro-9-fluoro-2-methyl-5,6-dihydro-4H-benzo[de][1,6]naphthyridin-4-yl)ethyl)pyridin-2-amine

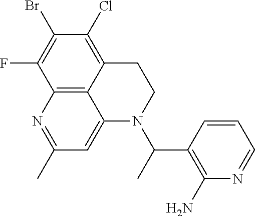 [SMILES: Cc1cc2c3c(c(Cl)c(Br)c(F)c3n1)CCN2C(C)c1cccnc1N]

To a vial containing 3-(1-(8-bromo-7-chloro-9-fluoro-2-methyl-5,6-dihydro-4H-benzo[de][1,6]naphthyridin-4-yl)ethyl)-N,N-bis(4-methoxybenzyl)pyridin-2-amine (300 mg, 0.444 mmol) was added TFA (1.4 mL, 17.75 mmol) and the reaction mixture was stirred at 90° C. for 1 h. The volatiles were removed in vacuo. The residue was dissolved in EtOAc and neutralized with sat. NaHCO₃ to pH 7. The resulting precipitate was collected via filtration and dried under vacuum. The filtrate was concentrated, combined with the solid and purified by FCC (eluting with a gradient of 0-100% EtOAc in hexanes) to give the title compound as a white solid (160 mg, 83%). LCMS calc. for C₁₉H₁₃BrClFN₄ (M+H)⁺ m/z=435.0, 437.0; found 435.0, 437.0.

### Step 3. 2-Amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

A mixture of 3-(1-(8-bromo-7-chloro-9-fluoro-2-methyl-5,6-dihydro-4H-benzo[de][1,6]naphthyridin-4-yl)ethyl)pyridin-2-amine (30 mg, 0.069 mmol), tert-butyl (3-cyano-4-(5,5-dimethyl-1,3,2-dioxaborinan-2-yl)-7-fluorobenzo[b]thiophen-2-yl)carbamate (55.7 mg, 0.138 mmol), Pd(dppf)Cl₂·CH₂Cl₂ (11.25 mg, 0.014 mmol) and K₂CO₃ (47.6 mg, 0.344 mmol) in 1,4-dioxane (1 mL) was stirred at 100° C. for 1 h 20 min. The reaction mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 1a. Diastereomer 1. Peak 1. LCMS calc. for C₂₈H₂₂ClF₂N₆S (M+H)⁺ m/z=547.1; found 547.1. This is the potent peak.

Example 1b. Diastereomer 2. Peak 2. LCMS calc. for C₂₈H₂₂ClF₂N₆S (M+H)⁺ m/z=547.1; found 547.1.

The compounds are understood to comprise mixtures of diastereoisomers, i.e., 2-amino-4-((Rₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4-((Sₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Sₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4-((Rₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile.

### Example 2a and 2b. 4-(4-(1-(2-Aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine

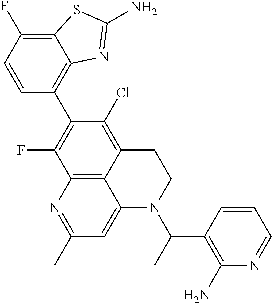 [SMILES: Cc1cc2c3c(c(Cl)c(-c4ccc(F)c5sc(N)nc45)c(F)c3n1)CCN2C(C)c1cccnc1N]

A mixture of 3-(1-(8-bromo-7-chloro-9-fluoro-2-methyl-5,6-dihydro-4H-benzo[de][1,6]naphthyridin-4-yl)ethyl)pyridin-2-amine (Example 1, Step 2, 22 mg, 0.050 mmol), (2-((tert-butoxycarbonyl)amino)-7-fluorobenzo[d]thiazol-4-yl)boronic acid (20.49 mg, 0.066 mmol), Pd(dppf)Cl₂·CH₂Cl₂ (4.12 mg, 5.05 μmol) and K₃PO₄ (32.2 mg, 0.151 mmol) in 1,4-dioxane (1 mL)/Water (0.200 mL) was stirred at 100° C. for 1.5 h. The reaction mixture was filtered and concentrated. The crude product was treated with 1:1 DCM/TFA (1 mL) at rt for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 2a. Diastereomer 1. Peak 1. LCMS calc. for C₂₆H₂₂ClF₂N₆S (M+H)⁺ m/z=523.1; found 523.1. This is the potent peak.

Example 2b. Diastereomer 2. Peak 2. LCMS calc. for C₂₆H₂₂ClF₂N₆S (M+H)⁺ m/z=523.1; found 523.1.

The compounds are understood to comprise mixtures of diastereoisomers, i.e., 4-((Rₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine, and 4-((Sₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine; and 4-((Rₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine, and 4-((Sₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine.

### Example 3a and 3b. 8-(4-(1-(2-Aminopyridin-3-yl)ethyl)-7-chloro-9-fluoro-2-methyl-5,6-dihydro-4H-benzo[de][1,6]naphthyridin-8-yl)-1-naphthonitrile

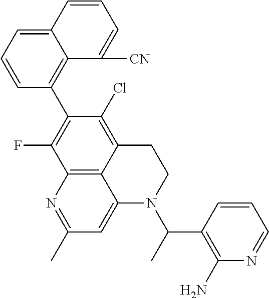 [SMILES: Cc1cc2c3c(c(Cl)c(-c4cccc5cccc(C#N)c45)c(F)c3n1)CCN2C(C)c1cccnc1N]

The title compound was prepared using an analogous procedure to Example 2, using the appropriate boronic acid coupling partner.

Example 3a. Diastereomer 1. Peak 1. LCMS calc. for C₃₀H₂₄ClFN₅ (M+H)⁺ m/z=508.1; found 508.1. This is the potent peak.

Example 3b. Diastereomer 2. Peak 2. LCMS calc. for C₃₀H₂₄ClFN₅ (M+H)⁺ m/z=508.1; found 508.1.

The compounds are understood to comprise mixtures of diastereoisomers, i.e., 8-((Rₐ)-4-((R)-1-(2-aminopyridin-3-yl)ethyl)-7-chloro-9-fluoro-2-methyl-5,6-dihydro-4H-benzo[de][1,6]naphthyridin-8-yl)-1-naphthonitrile, and 8-((Sₐ)-4-((S)-1-(2-aminopyridin-3-yl)ethyl)-7-chloro-9-fluoro-2-methyl-5,6-dihydro-4H-benzo[de][1,6]naphthyridin-8-yl)-1-naphthonitrile; and 8-((Rₐ)-4-((S)-1-(2-aminopyridin-3-yl)ethyl)-7-chloro-9-fluoro-2-methyl-5,6-dihydro-4H-benzo[de][1,6]naphthyridin-8-yl)-1-naphthonitrile, and 8-((Sₐ)-4-((R)-1-(2-aminopyridin-3-yl)ethyl)-7-chloro-9-fluoro-2-methyl-5,6-dihydro-4H-benzo[de][1,6]naphthyridin-8-yl)-1-naphthonitrile.

### Example 4a and 4b. 3-(1-(7-Chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine

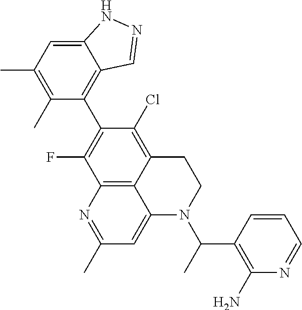 [SMILES: Cc1cc2c3c(c(Cl)c(-c4c(C)c(C)cc5[nH]ncc45)c(F)c3n1)CCN2C(C)c1cccnc1N]

The title compound was prepared using an analogous procedure to Example 2, using the appropriate boronic acid coupling partner.

Example 4a. Diastereomer 1. Peak 1. LCMS calc. for C₂₈H₂₇ClFN₆ (M+H)⁺ m/z=501.1; found 501.1. This is the potent peak.

Example 4b. Diastereomer 2. Peak 2. LCMS calc. for C₂₈H₂₇ClFN₆ (M+H)⁺ m/z=501.1; found 501.1.

The compounds are understood to comprise mixtures of diastereoisomers, i.e., 3-((Rₐ)-1-((R)-7-chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine, and 3-((Sₐ)-1-((S)-7-chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine; and 3-((Rₐ)-1-((S)-7-chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine, and 3-((Sₐ)-1-((R)-7-chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine.

### Example 5a and 5b. 2-Amino-4-(7-chloro-9-fluoro-2-methyl-4-((R)-1-(pyridin-3-yl)ethyl)-5,6-dihydro-4H-benzo[de][1,6]naphthyridin-8-yl)-7-fluorobenzo[b]thiophene-3-carbonitrile

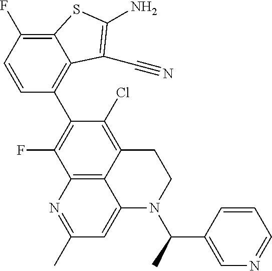 [SMILES: Cc1cc2c3c(c(Cl)c(-c4ccc(F)c5sc(N)c(C#N)c45)c(F)c3n1)CCN2[C@H](C)c1cccnc1]

### Step 1. (R)-8-Bromo-7-chloro-9-fluoro-2-methyl-4-(1-(pyridin-3-yl)ethyl)-5,6-dihydro-4H-benzo[de][1,6]naphthyridine

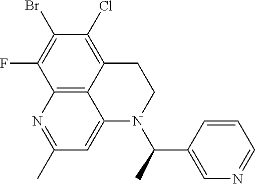 [SMILES: Cc1cc2c3c(c(Cl)c(Br)c(F)c3n1)CCN2[C@H](C)c1cccnc1]

(R)-1-(Pyridin-3-yl)ethan-1-amine hydrochloride (18 mg, 0.113 mmol) was added to a solution of 2-(7-bromo-4,6-dichloro-8-fluoro-2-methylquinolin-5-yl)acetaldehyde (Intermediate 2; 36 mg, 0.103 mmol) in THF (500 μL). The mixture was stirred at r.t. for 30 min. To another vial was added NaBH(OAc)₃ (87 mg, 0.410 mmol), DCM (500 μL) and TFA (32 μL, 0.41 mmol) and stirred for 30 min. The second solution was then added to the first slowly.

The resulting mixture was stirred at r.t. for 3 h. The reaction mixture was diluted with sat. aq. NaHCO₃ and DCM. The aqueous layer was extracted with DCM. The organic layer was dried over Na₂SO₄, filtered and concentrated. The residue was purified by FCC (0-70% EtOAc in hexanes) to give the title compound as a colorless foam (30 mg, 70%). LCMS calc. for C₁₉H₁₇BrClFN₃ (M+H)⁺ m/z=420.0, 422.0; found 420.0, 422.0.

### Step 2. 2-Amino-4-(7-chloro-9-fluoro-2-methyl-4-((R)-1-(pyridin-3-yl)ethyl)-5,6-dihydro-4H-benzo[de][1,6]naphthyridin-8-yl)-7-fluorobenzo[b]thiophene-3-carbonitrile

A mixture of (R)-8-bromo-7-chloro-9-fluoro-2-methyl-4-(1-(pyridin-3-yl)ethyl)-5,6-dihydro-4H-benzo[de][1,6]naphthyridine (38 mg, 0.090 mmol), tert-butyl (3-cyano-4-(5,5-dimethyl-1,3,2-dioxaborinan-2-yl)-7-fluorobenzo[b]thiophen-2-yl)carbamate (73.0 mg, 0.181 mmol), Pd(dppf)Cl₂·CH₂Cl₂ (14.75 mg, 0.018 mmol) and K₂CO₃ (62.4 mg, 0.452 mmol) in 1,4-dioxane (1 mL) was stirred at 100° C. for 1 h 20 min. The reaction mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 5a. Diastereomer 1. Peak 1. LCMS calc. for C₂₈H₂₁ClF₂N₅S (M+H)⁺ m/z=532.1; found 532.1. This is the potent peak.

Example 5b. Diastereomer 2. Peak 2. LCMS calc. for C₂₈H₂₁ClF₂N₅S (M+H)⁺ m/z=532.1; found 532.1.

The compounds are understood to be the atropisomers, i.e., 2-amino-(Rₐ)-4-(7-chloro-9-fluoro-2-methyl-4-((R)-1-(pyridin-3-yl)ethyl)-5,6-dihydro-4H-benzo[de][1,6]naphthyridin-8-yl)-7-fluorobenzo[b]thiophene-3-carbonitrile; and 2-amino-(Sₐ)-4-(7-chloro-9-fluoro-2-methyl-4-((R)-1-(pyridin-3-yl)ethyl)-5,6-dihydro-4H-benzo[de][1,6]naphthyridin-8-yl)-7-fluorobenzo[b]thiophene-3-carbonitrile.

The alternative stereoisomers, i.e., 2-amino-(Rₐ)-4-(7-chloro-9-fluoro-2-methyl-4-((S)-1-(pyridin-3-yl)ethyl)-5,6-dihydro-4H-benzo[de][1,6]naphthyridin-8-yl)-7-fluorobenzo[b]thiophene-3-carbonitrile; and 2-amino-(Sₐ)-4-(7-chloro-9-fluoro-2-methyl-4-((S)-1-(pyridin-3-yl)ethyl)-5,6-dihydro-4H-benzo[de][1,6]naphthyridin-8-yl)-7-fluorobenzo[b]thiophene-3-carbonitrile; are prepared by an analogous procedure using (S)-1-(pyridin-3-yl)ethan-1-amine hydrochloride as a starting material.

### Example 6a and 6b. 2-Amino-4-(4-((R)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

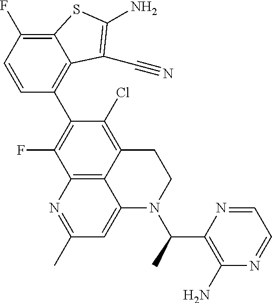 [SMILES: Cc1cc2c3c(c(Cl)c(-c4ccc(F)c5sc(N)c(C#N)c45)c(F)c3n1)CCN2[C@H](C)c1nccnc1N]

### Step 1. (R)-3-(1-Aminoethyl)-N,N-bis(4-methoxybenzyl)pyrazin-2-amine

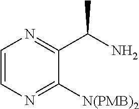

To a solution of 1-(3-(bis(4-methoxybenzyl)amino)pyrazin-2-yl)ethan-1-one (0.66 g, 1.749 mmol) and (R)-2-methylpropane-2-sulfinamide (0.318 g, 2.62 mmol) in THF (4.4 mL) was added titanium(IV) ethoxide (0.92 mL, 4.37 mmol). The resulting mixture was stirred at 80° C. for 16 h. The reaction mixture was diluted with ice water and stirred for 10 min. The reaction mixture was filtered through a pad of diatomaceous earth and washed with EtOAc. The filtrate was separated. The organic layer was dried over Na₂SO₄ and concentrated. The crude product was purified by FCC (0-60% EtOAc in hexanes). This intermediate was placed in a vial and dissolved in THF (6 mL), followed by addition of NaBH₄ (0.112 g, 2.97 mmol) in water (1 mL) at −78° C. dropwise. The resulting mixture was warmed to r.t. slowly and stirred for 3 h. EtOAc was added and the layers were separated. The organic layer was dried over Na₂SO₄ and concentrated and the crude product was dissolved in DCM (2 mL) and TFA (0.16 mL, 2.098 mmol) was added. The reaction mixture was stirred at 45° C. for 45 min. The reaction mixture was diluted with DCM and sat. aq. NaHCO₃. The organic layer was separated and concentrated. The crude product was purified by FCC (0-100% EtOAc in hexanes) to provide the title compound (100 mg, 15% over three steps). LCMS calc. for C₂₂H₂₇N₄O₂ (M+H)⁺ m/z=379.2; found 379.2.

### Step 2. (R)-3-(1-(8-Bromo-7-chloro-9-fluoro-2-methyl-5,6-dihydro-4H-benzo[de][1,6]naphthyridin-4-yl)ethyl)-N,N-bis(4-methoxybenzyl)pyrazin-2-amine

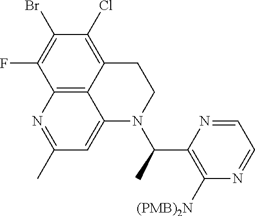

(R)-3-(1-Aminoethyl)-N,N-bis(4-methoxybenzyl)pyrazin-2-amine (42.7 mg, 0.113 mmol) was added to a solution of 2-(7-bromo-4,6-dichloro-8-fluoro-2-methylquinolin-5-yl)acetaldehyde (Intermediate 2, 36 mg, 0.103 mmol) in THF (500 μL). The mixture was stirred at r.t. for 30 min. To a separate vial were added NaBH(OAc)₃ (87 mg, 0.410 mmol), DCM (500 μL) and TFA (31.6 μL, 0.410 mmol) and the mixture was stirred for 30 min., then added to the first reaction mixture slowly. The resulting mixture was stirred at r.t. for 3 h, then diluted with sat. aq. NaHCO₃ and DCM. The aqueous layer was extracted with DCM and the combined organics were dried over Na₂SO₄ and concentrated. The residue was purified by FCC (0-70% EtOAc in hexanes) to give the title compound as a colorless foam (assumed quantitative for next step). LCMS calc. for C₃₄H₃₃BrClFN₅O₂(M+H)⁺ m/z=676.1, 678.1; found 676.1, 678.1.

### Step 3. (R)-3-(1-(8-Bromo-7-chloro-9-fluoro-2-methyl-5,6-dihydro-4H-benzo[de][1,6]naphthyridin-4-yl)ethyl)pyrazin-2-amine

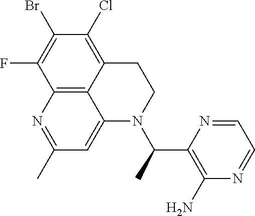 [SMILES: Cc1cc2c3c(c(Cl)c(Br)c(F)c3n1)CCN2[C@H](C)c1nccnc1N]

To a vial containing (R)-3-(1-(8-bromo-7-chloro-9-fluoro-2-methyl-5,6-dihydro-4H-benzo[de][1,6]naphthyridin-4-yl)ethyl)-N,N-bis(4-methoxybenzyl)pyrazin-2-amine (69 mg, 0.102 mmol) was added TFA (314 μL, 4.08 mmol) and the reaction mixture was stirred at 90° C. for 1 h. The volatiles was removed in vacuo. The residue was dissolved in EtOAc and neutralized with sat. NaHCO₃ to pH 7. The resulting precipitate was collected vial filtration and dried under vacuum, then purified by FCC (0-100% EtOAc in hexanes) to provide the title compound (40 mg, 90%). LCMS calc. for C₁₃H₁₇BrClFN₅ (M+H)⁺ m/z=436.0, 438.0; found 436.0, 438.0.

### Step 4. 2-amino-4-(4-((R)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

A mixture of the product from the previous step (40 mg, 0.092 mmol), tert-butyl (3-cyano-4-(5,5-dimethyl-1,3,2-dioxaborinan-2-yl)-7-fluorobenzo[b]thiophen-2-yl)carbamate (74.1 mg, 0.183 mmol), Pd(dppf)Cl₂·CH₂Cl₂ (14.96 mg, 0.018 mmol) and K₂CO₃ (63.3 mg, 0.458 mmol) in 1,4-dioxane (1 mL) was stirred at 100° C. for 1 h 20 min. The reaction mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 6a. Diastereomer 1. Peak 1. LCMS calc. for C₂₇H₂₁ClF₂N₇S (M+H)⁺ m/z=548.1; found 548.1. This is the potent peak. ¹H NMR (500 Hz, DMSO) b 8.02 (s, 2H), 7.95 (d, J=2.7 Hz, 1H), 7.80 (d, J=2.7 Hz, 1H), 7.21 (dd, J=8.4, 5.2 Hz, 1H), 7.13 (t, J=8.9 Hz, 1H), 6.79 (s, 1H), 6.12 (s, 2H), 5.43 (q, J=6.7 Hz, 1H), 3.54 (ddd, J=12.8, 8.2, 4.8 Hz, 1H), 3.45 (ddd, J=12.3, 7.2, 5.1 Hz, 1H), 3.10 (dt, J=16.8, 5.8 Hz, 1H), 3.01 (ddd, J=16.5, 8.4, 5.2 Hz, 1H), 2.50 (s, 3H), 1.61 (d, J=6.6 Hz, 3H).

Example 6b. Diastereomer 2. Peak 2. LCMS calc. for C₂₇H₂₁ClF₂N₇S (M+H)⁺ m/z=548.1; found 548.1.

The compounds are understood to be the atropisomers, i.e., 2-amino-(Rₐ)-4-(4-((R)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; and 2-amino-(Sₐ)-4-(4-((R)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile.

The alternative stereoisomers, i.e., 2-amino-(Rₐ)-4-(4-((S)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; and 2-amino-(Sₐ)-4-(4-((S)-1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; are prepared by an analogous procedure using (S)-2-methylpropane-2-sulfinamide as a starting material.

### Example 7a and 7b. 2-Amino-4-(4-(1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

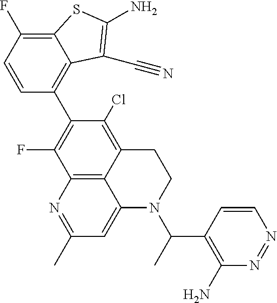 [SMILES: Cc1cc2c3c(c(Cl)c(-c4ccc(F)c5sc(N)c(C#N)c45)c(F)c3n1)CCN2C(C)c1ccnnc1N]

### Step 1. 4-(1-Aminoethyl)-N-(4-methoxybenzyl)pyridazin-3-amine

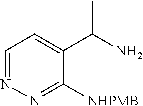

To a solution of 3-((4-methoxybenzyl)amino)pyridazine-4-carbonitrile (596 mg, 2.48 mmol) in THF (10 mL) was added methylmagnesium bromide (3.0 M in diethyl ether, 1.7 mL, 5.09 mmol) dropwise at 0° C. under an atmosphere of N₂. The resulting mixture was stirred at 0° C. for 5 min. before being warmed to r.t. The reaction mixture was stirred overnight. The reaction was quenched with MeOH (1 mL) then NaBH₄ (94 mg, 2.481 mmol) was added in a single portion. The mixture was stirred at r.t. for 4 h. The reaction was quenched with sat. aq. NaHCO₃ and extracted with EtOAc. The organic layer was dried over Na₂SO₄ and concentrated. The residue was purified by FCC (0-100% EtOAc in hexanes) to provide the title compound (350 mg, 55%). LCMS calc. for C₁₄H₁₉N₄O (M+H)⁺ m/z=259.1; found 259.1.

### Step 2. 2-Amino-4-(4-(1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

The title compound was prepared according to the procedure described in Example 5, Steps 1-2, using 4-(1-aminoethyl)-N-(4-methoxybenzyl)pyridazin-3-amine in the reductive amination step. The compound was isolated as two peaks

Example 7a. Diastereomer 1. Peak 1. LCMS calc. for C₂₇H₂₁ClF₂N₇S (M+H)⁺ m/z=548.1; found 548.1. This is the potent peak.

Example 7b. Diastereomer 2. Peak 2. LCMS calc. for C₂₇H₂₁ClF₂N₇S (M+H)⁺ m/z=548.1; found 548.1.

The compounds are understood mixtures of diastereoisomers, i.e., 2-amino-4-((Rₐ)-4-((R)-1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4-((Sₐ)-4-((S)-1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Sₐ)-4-((R)-1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4-((Rₐ)-4-((S)-1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile.

### Example 8a and 8b. 2-Amino-4-(4-(1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

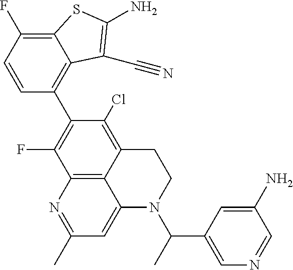 [SMILES: Cc1cc2c3c(c(Cl)c(-c4ccc(F)c5sc(N)c(C#N)c45)c(F)c3n1)CCN2C(C)c1cncc(N)c1]

### Step 1. 1-(5-Nitropyridin-3-yl)ethan-1-amine

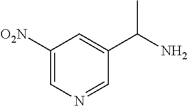

A solution of 1-(5-nitropyridin-3-yl)ethan-1-one (380 mg, 2.287 mmol) and ammonia (7.0 N in MeOH, 1.3 mL, 9.15 mmol) and titanium(IV) isopropoxide (1.0 mL, 3.43 mmol) in THF (11 mL) was stirred at 65° C. for 48 hf. Then NaBH₄ (108 mg, 2.86 mmol) was added and the mixture stirred at 0° C. for 1 h. After completion, the reaction mixture was diluted with water and extracted with EtOAc. The combined organic layers were dried over anhydrous Na₂SO₄ and concentrated under vacuum. The residue was purified by FCC (0-90% EtOAc in hexanes) to afford the title compound (302 mg, 1.807 mmol, 79%) as a yellow oil. LCMS calc. for C₇H₁₀N₃O₂ (M+H)⁺ m/z=168.1; found 168.1.

### Step 2. 3-(1-(8-Bromo-7-chloro-9-fluoro-2-methyl-5,6-dihydro-4H-benzo[de][1,6]naphthyridin-4-yl)ethyl)-5-nitropyridin-2-amine

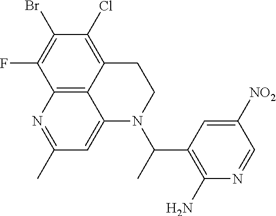 [SMILES: Cc1cc2c3c(c(Cl)c(Br)c(F)c3n1)CCN2C(C)c1cc([N+](=O)[O-])cnc1N]

1-(5-Nitropyridin-3-yl)ethan-1-amine (19.91 mg, 0.119 mmol) and 2-(7-bromo-4,6-dichloro-8-fluoro-2-methylquinolin-5-yl)acetaldehyde (Intermediate 2, 38 mg, 0.108 mmol) in THF (500 μL) were stirred at r.t. for 30 min. To another vial were added NaBH(OAc)₃ (92 mg, 0.433 mmol), DCM (500 μL) and TFA (33.4 μL, 0.433 mmol) and stirred for 30 min., then transferred to the first vial and stirred at r.t. for 3 h. The reaction mixture was diluted with sat. NaHCO₃ solution and DCM. The aqueous layer was extracted with DCM. The combined organics were dried over Na₂SO₄, filtered and concentrated. The crude product was dissolved in MeCN (4 mL), NEt₃ (30.2 μL, 0.217 mmol) was added to reaction vial. The reaction mixture was stirred at 70° C. for 30 min. After cooling to r.t., the volatiles was removed in vacuo. The residue was purified by FCC (0-70% EtOAc in hexanes) to give the title compound as a colorless foam (44 mg, 87%). LCMS calc. for C₁₉H₁₇BrClFN₅O₂(M+H)⁺ m/z=480.0, 482.0; found 480.0, 482.0.

### Step 3. 3-(1-(8-Bromo-7-chloro-9-fluoro-2-methyl-5,6-dihydro-4H-benzo[de][1,6]naphthyridin-4-yl)ethyl)pyridine-2,5-diamine

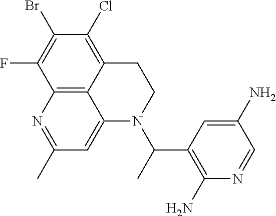 [SMILES: Cc1cc2c3c(c(Cl)c(Br)c(F)c3n1)CCN2C(C)c1cc(N)cnc1N]

In a 40 mL vial, 8-bromo-7-chloro-9-fluoro-2-methyl-4-(1-(5-nitropyridin-3-yl)ethyl)-5,6-dihydro-4H-benzo[de][1,6]naphthyridine (44 mg, 0.094 mmol), NH₄Cl (53.1 mg, 0.992 mmol), and iron (52.8 mg, 0.945 mmol) were dissolved in THF (0.5 mL) open to air. The mixture was stirred at 60° C. for 2 h, then filtered through a plug of diatomaceous earth. The filtrate was concentrated and the crude product was used in the next step without further purification. LCMS calc. for C₁₉H₁₉BrClFN₅ (M+H)⁺ m/z=450.0, 452.0; found 450.0, 452.0.

### Step 4. 2-Amino-4-(4-(1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile

A mixture of 5-(1-(8-bromo-7-chloro-9-fluoro-2-methyl-5,6-dihydro-4H-benzo[de][1,6]naphthyridin-4-yl)ethyl)pyridin-3-amine (31 mg, 0.071 mmol), tert-butyl (3-cyano-4-(5,5-dimethyl-1,3,2-dioxaborinan-2-yl)-7-fluorobenzo[b]thiophen-2-yl)carbamate (57.5 mg, 0.142 mmol), Pd(dppf)Cl₂·CH₂Cl₂ (11.62 mg, 0.014 mmol) and K₂CO₃ (49.2 mg, 0.356 mmol) in 1,4-dioxane (1 mL) was stirred at 100° C. for 2 h then 110° C. for 3 h. The reaction mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 8a. Diastereomer 1. Peak 1. LCMS calc. for C₂₈H₂₂ClF₂N₆S (M+H)⁺ m/z=547.1; found 547.1. This is the potent peak.

Example 8b. Diastereomer 2. Peak 2. LCMS calc. for C₂₈H₂₂ClF₂N₆S (M+H)⁺ m/z=547.1; found 547.1.

The compounds are understood to comprise mixtures of diastereoisomers, i.e., 2-amino-4-((Rₐ)-4-((R)-1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4—((Sₐ)-4-((S)-1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Sₐ)-4-((R)-1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile, and 2-amino-4-((Rₐ)-4-((S)-1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile.

### Example 9a and 9b. 2-Amino-4-(7-chloro-9-fluoro-2-methyl-4-(1-(1-methyl-1H-pyrazol-5-yl)ethyl)-5,6-dihydro-4H-benzo[de][1,6]naphthyridin-8-yl)-7-fluorobenzo[b]thiophene-3-carbonitrile

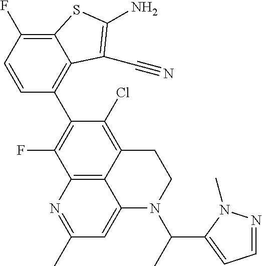 [SMILES: Cc1cc2c3c(c(Cl)c(-c4ccc(F)c5sc(N)c(C#N)c45)c(F)c3n1)CCN2C(C)c1ccnn1C]

### Step 1. (R)-1-(1-Methyl-1H-pyrazol-5-yl)ethan-1-amine

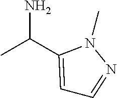

To a solution of 1-(1-methyl-1H-pyrazol-5-yl)ethan-1-one (574 mg, 4.62 mmol) and (R)-2-methylpropane-2-sulfinamide (841 mg, 6.94 mmol) in THF (11 mL) was added titanium(IV) ethoxide (2.4 mL, 11.56 mmol). The resulting mixture was stirred at 80° C. for 16 h. The reaction mixture was diluted with ice water and stirred for 10 min., then filtered through a pad of diatomaceous earth and washed with EtOAc. The filtrate was separated. The organic layer was dried over Na₂SO₄ and concentrated. To a vial charged with this intermediate in THF (6 mL) was added NaBH₄ (297 mg, 7.86 mmol) in water (1 mL) at −78° C. dropwise. The resulting mixture was warmed to r.t. slowly and stirred for 3 h. To the reaction vial were added water and EtOAc. The layers were separated and the organic layer was dried over Na₂SO₄ and concentrated. The crude product was dissolved in DCM (2 mL) and HCl (4.0 M in 1.4-dioxane, 4.6 mL, 18.49 mmol) was added. After stirring at r.t. for 2 h, ether was added and the resulting precipitate was collected by filtration to provide the title compound as the corresponding HCl salt, as a 1:1 mixture of diastereoisomers (300 mg, 52% over three steps).

### Step 2. 2-Amino-4-(7-chloro-9-fluoro-2-methyl-4-(1-(1-methyl-1H-pyrazol-5-yl)ethyl)-5,6-dihydro-4H-benzo[de][1,6]naphthyridin-8-yl)-7-fluorobenzo[b]thiophene-3-carbonitrile

The title compound was prepared in an analogous fashion to Example 5, Steps 1-2, using the amine from Step 1 in the reductive amination step.

Example 9a. Diastereomer 1. Peak 1. LCMS calc. for C₂₇H₂₂ClF₂N₆S (M+H)⁺ m/z=535.1; found 535.1. This is the potent peak.

Example 9b. Diastereomer 2. Peak 2. LCMS calc. for C₂₇H₂₂ClF₂N₆S (M+H)⁺ m/z=535.1; found 535.1.

The compounds are understood to comprise mixtures of diastereoisomers, i.e., 2-amino-4-((Rₐ)-7-chloro-9-fluoro-2-methyl-4-((R)-1-(1-methyl-1H-pyrazol-5-yl)ethyl)-5,6-dihydro-4H-benzo[de][1,6]naphthyridin-8-yl)-7-fluorobenzo[b]thiophene-3-carbonitrile, and 2-amino-4-((Sₐ)-7-chloro-9-fluoro-2-methyl-4-((S)-1-(1-methyl-1H-pyrazol-5-yl)ethyl)-5,6-dihydro-4H-benzo[de][1,6]naphthyridin-8-yl)-7-fluorobenzo[b]thiophene-3-carbonitrile; and 2-amino-4-((Sₐ)-7-chloro-9-fluoro-2-methyl-4-((R)-1-(1-methyl-1H-pyrazol-5-yl)ethyl)-5,6-dihydro-4H-benzo[de][1,6]naphthyridin-8-yl)-7-fluorobenzo[b]thiophene-3-carbonitrile, and 2-amino-4-((Rₐ)-7-chloro-9-fluoro-2-methyl-4-((S)-1-(1-methyl-1H-pyrazol-5-yl)ethyl)-5,6-dihydro-4H-benzo[de][1,6]naphthyridin-8-yl)-7-fluorobenzo[b]thiophene-3-carbonitrile.

### Example 10a and 10b. 2-Amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile

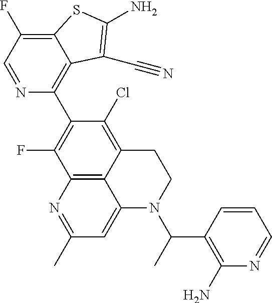 [SMILES: Cc1cc2c3c(c(Cl)c(-c4ncc(F)c5sc(N)c(C#N)c45)c(F)c3n1)CCN2C(C)c1cccnc1N]

### Step 1. tert-Butyl (4-chloro-3-cyano-7-fluorothieno[3,2-c]pyridin-2-yl)carbamate

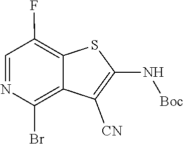 [SMILES: CC(C)(C)OC(=O)Nc1sc2c(F)cnc(Br)c2c1C#N]

2-Amino-4-chloro-7-fluorothieno[3,2-c]pyridine-3-carbonitrile (286 mg, 1.25 mmol), Boc₂O (0.438 mL, 1.885 mmol), DMAP (30.7 mg, 0.251 mmol), and DIPEA (0.329 mL, 1.885 mmol) were dissolved in THF (6 mL). The mixture was stirred at 60° C. for 3 h. Volatiles were removed and the mixture was purified by FCC (0-50% EtOAc in hexanes) to provide the title compound (412 mg, ˜100%). LCMS calc. for C₁₃H₁₂ClFN₃O₂S (M+H)⁺ m/z=358.1; found 358.1.

### Step 2. tert-Butyl (4-chloro-3-cyano-7-fluorothieno[3,2-c]pyridin-2-yl)((2-(trimethylsilyl)ethoxy)methyl)carbamate

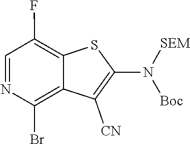 [SMILES: CC(C)(C)OC(=O)N(COCC[Si](C)(C)C)c1sc2c(F)cnc(Br)c2c1C#N]

To a vial containing tert-butyl (4-chloro-3-cyano-7-fluorothieno[3,2-c]pyridin-2-yl)carbamate (412 mg, 1.257 mmol) in DMF (8.4 mL) at 0° C. was added NaH (60% in mineral oil, 55.3 mg, 1.383 mmol) and stirred at r.t. for 30 min. The reaction vial was cooled to 0° C. 2-(Trimethylsilyl)ethoxymethyl chloride (297 μL, 1.508 mmol) was added and the reaction mixture was stirred at r.t. for 90 min. The reaction was quenched with water and the resulting mixture was extracted with EtOAc. The organic layer was dried over Na₂SO₄ and concentrated. The crude product was purified by FCC (0-30% EtOAc in hexanes) to provide the title compound (393 mg, 68%). LCMS calc. for C₁₉H₂₆ClFN₃O₃SSi (M+H)⁺ m/z=458.1; found 458.1.

### Step 3. tert-Butyl (4-(4-(1-(2-(bis(4-methoxybenzyl)amino)pyridin-3-yl)ethyl)-7-chloro-9-fluoro-2-methyl-5,6-dihydro-4H-benzo[de][1,6]naphthyridin-8-yl)-3-cyano-7-fluorothieno[3,2-c]pyridin-2-yl)((2-(trimethylsilyl)ethoxy)methyl)carbamate

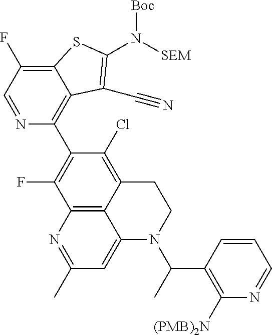

To a solution of 3-(1-(8-bromo-7-chloro-9-fluoro-2-methyl-5,6-dihydro-4H-benzo[de][1,6]naphthyridin-4-yl)ethyl)-N,N-bis(4-methoxybenzyl)pyridin-2-amine (prepared according to Example 1, Step 2, using racemic amine; 123 mg, 0.183 mmol) in THF (1 mL) under N₂ was added iPrMgCl·LiCl (1.3 M in THF, 153 μL, 0.199 mmol) at −78° C. and the mixture was stirred at −78° C. for 40 min. Then ZnCl₂ (1.9 M in THF, 105 μL, 0.199 mmol) was added at −78° C. The mixture was stirred at −78° C. for 5 min. and stirred at 25° C. for 30 min. The mixture was transferred into a solution of CPhos Pd G3 (26.8 mg, 0.033 mmol) and tert-butyl (4-chloro-3-cyano-7-fluorothieno[3,2-c]pyridin-2-yl)((2-(trimethylsilyl)ethoxy)methyl)carbamate (76 mg, 0.166 mmol) in THF (1 mL) and stirred at 60° C. for 1 h. After completion, the reaction mixture was diluted with EtOAc and washed with water. The organic layer was dried over anhydrous Na₂SO₄ and concentrated under vacuum. The residue was purified by FCC (0-100% EtOAc in hexanes) to provide the title compound. LCMS calc. for C₅₄H₅₉ClF₂N₇O₅SSi (M+H)⁺ m/z=1018.4; found 1018.4.

### Step 4. 2-Amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile

To a vial containing tert-butyl (4-(4-(1-(2-(bis(4-methoxybenzyl)amino)pyridin-3-yl)ethyl)-7-chloro-9-fluoro-2-methyl-5,6-dihydro-4H-benzo[de][1,6]naphthyridin-8-yl)-3-cyano-7-fluorothieno[3,2-c]pyridin-2-yl)((2-(trimethylsilyl)ethoxy)methyl)carbamate (8.5 mg, 8.34 μmol) was added HFIP (0.25 mL) and TFA (0.25 mL). The resulting mixture was heated at 70° C. for 45 min. then at 80° C. for an additional 45 min. After cooling to r.t., the solvent was evaporated. The residue was diluted with MeCN and purified using prep.-LCMS (XBRIDGE® C₁₈ column, eluting with a gradient of MeCN/water containing 0.1% TFA, at flow rate of 60 mL/min.). Additional purification was performed using prep.-LCMS (XBRIDGE® C₁₈ column, eluting with a gradient of MeCN/water containing 0.1% NH₄OH, at flow rate of 60 mL/min.) to afford the title compound as two peaks.

Example 10a. Diastereomer 1. Peak 1. LCMS calc. for C₂₇H₂₁ClF₂N₇S (M+H)⁺ m/z=548.1; found 548.1. This is the potent peak.

Example 10b. Diastereomer 2. Peak 2. LCMS calc. for C₂₇H₂₁ClF₂N₇S (M+H)⁺ m/z=548.1; found 548.1.

The compounds are understood to comprise mixtures of diastereoisomers, i.e., 2-amino-4-((Rₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile, and 2-amino-4-((Sₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile; and 2-amino-4-((Sₐ)-4-((R)-1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile, and 2-amino-4-((Rₐ)-4-((S)-1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile.

### Compound 11a, 11b and 11c. 2-Amino-4-(4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile

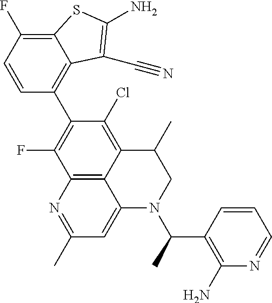 [SMILES: Cc1cc2c3c(c(Cl)c(-c4ccc(F)c5sc(N)c(C#N)c45)c(F)c3n1)C(C)CN2[C@H](C)c1cccnc1N]

### Step 1. 2-(7-Bromo-4,6-dichloro-8-fluoro-2-methylquinolin-5-yl)propanenitrile

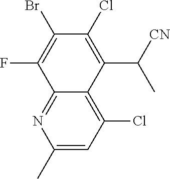 [SMILES: Cc1cc(Cl)c2c(C(C)C#N)c(Cl)c(Br)c(F)c2n1]

In a 40 mL vial, 2-(7-bromo-4,6-dichloro-8-fluoro-2-methylquinolin-5-yl)MeCN (230 mg, 0.661 mmol) was dissolved in THF (3.3 mL) under N₂ and then cooled to −78° C. LiHMDS (1.0 M in THF, 730 μL, 0.730 mmol) was added to the reaction mixture dropwise at −78° C. The mixture was stirred 5 min. at −78° C., then methyl iodide (43 μL, 0.694 mmol) was added to the reaction mixture in one portion. The mixture was stirred for 15 min. at −78° C., warmed to 0° C. for 15 min., then the reaction was quenched by the addition of 1 M aq. NH₄Cl (5 mL) and the resulting mixture was extracted with DCM (3×5 mL). The combined organic layers were washed with brine, dried over MgSO₄, and solvent was removed in vacuo. The crude product was purified by FCC (eluting with of 0-30% heptane/EtOAc) to provide the title compound (180 mg, 75%). LCMS calc. for C₁₃H₉BrCl₂FN₂ (M+H)⁺ m/z=360.9, 362.9; found 360.9, 362.9.

### Step 2. 2-(7-Bromo-4,6-dichloro-8-fluoro-2-methylquinolin-5-yl)propanal

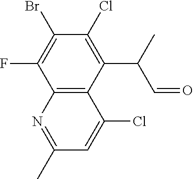 [SMILES: Cc1cc(Cl)c2c(C(C)C=O)c(Cl)c(Br)c(F)c2n1]

In a 40 mL vial, 2-(7-bromo-4,6-dichloro-8-fluoro-2-methylquinolin-5-yl)propanenitrile (89.5 mg, 0.247 mmol) was dissolved in toluene (2.5 mL) under N₂. The mixture was stirred until all substrate was dissolved and then cooled to −78° C. DIBAL-H (1.0 M in DCM, 400 μL, 0.400 mmol) was added slowly to the reaction mixture. The mixture was stirred for 1 h at −78° C. At this time, EtOAc (363 μL, 3.71 mmol) was dissolved in 1 mL of THF and the mixture was added slowly to the reaction mixture followed by a solution of MeOH (150 μL, 3.71 mmol) in THF (1 mL). The mixture was removed from the dry ice bath and then 5 mL of 3M citric acid solution was added over 2 min. The reaction mixture was diluted with water (3 mL) and EtOAc (8 mL) and stirred rapidly for 30 min. The layers were separated and the aqueous layer was extracted with EtOAc. The combined organics were washed with water and brine, dried over Na₂SO₄ and concentrated. The crude product was used in the next step without further purification (52 mg, 57%). LCMS calc. for C₁₃H₁₀BrCl₂FNO (M+H)⁺ m/z=363.9, 365.9; found 363.9, 365.9.

### Step 3. 3-((1R)-1-(8-Bromo-7-chloro-9-fluoro-2,6-dimethyl-5,6-dihydro-4H-benzo[de][1,6]naphthyridin-4-yl)ethyl)-N,N-bis(4-methoxybenzyl)pyridin-2-amine

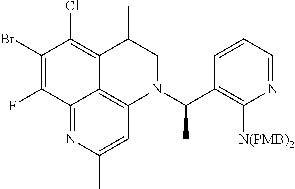

In a 40 mL vial, (R)-3-(1-aminoethyl)-N,N-bis(4-methoxybenzyl)pyridin-2-amine (55 mg, 0.145 mmol), 2-(7-bromo-4,6-dichloro-8-fluoro-2-methylquinolin-5-yl)propanal (53 mg, 0.145 mmol), NaBH₃CN (27 mg, 0.436 mmol), and AcOH (25 μL, 0.436 mmol) were dissolved in MeOH (2.2 mL) under N₂. The mixture was stirred at r.t. for 2.5 h. The reaction was quenched by the addition of water and brine (3 mL each) and the mixture was extracted with EtOAc (3×5 mL). The organic extract was washed with brine, dried over Na₂SO₄, and volatiles were removed in vacuo. The crude material was purified by FCC (eluting with 0-100% EtOAc/heptane) to provide the title compound (58 mg, 57%). LCMS calc. for C₃₆H₃₆BrClFN₄O₂(M+H)⁺ m/z=689.1, 691.1; found 689.1, 691.1.

### Step 4. 3-((1R)-1-(8-Bromo-7-chloro-9-fluoro-2,6-dimethyl-5,6-dihydro-4H-benzo[de][1,6]naphthyridin-4-yl)ethyl)pyridin-2-amine

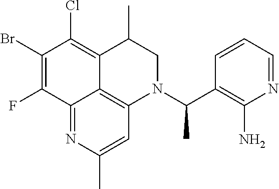 [SMILES: Cc1cc2c3c(c(Cl)c(Br)c(F)c3n1)C(C)CN2[C@H](C)c1cccnc1N]

In a 2 dram vial, 3-((1R)-1-(8-bromo-7-chloro-9-fluoro-2,6-dimethyl-5,6-dihydro-4H-benzo[de][1,6]naphthyridin-4-yl)ethyl)-N,N-bis(4-methoxybenzyl)pyridin-2-amine (9.5 mg, 0.014 mmol) was dissolved in TFA (0.25 mL) under N₂. The mixture was stirred at 90° C. for 1 h, then cooled to r.t. TFA was removed in vacuo. The crude product was converted to the free base by dissolving in DCM and washing with aq. NaHCO₃, extracting the aqueous layer with DCM drying with Na₂SO₄ and concentrating. The crude product was used directly in the next step. LCMS calc. for C₂₀H₂₀BrClFN₄ (M+H)⁺ m/z=449.0, 451.0 found 449.0, 451.0.

### Step 5. 2-Amino-4-(4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile

A mixture of 3-((1R)-1-(8-bromo-7-chloro-9-fluoro-2,6-dimethyl-5,6-dihydro-4H-benzo[de][1,6]naphthyridin-4-yl)ethyl)pyridin-2-amine (40 mg, 0.089 mmol), tert-butyl (3-cyano-4-(5,5-dimethyl-1,3,2-dioxaborinan-2-yl)-7-fluorobenzo[b]thiophen-2-yl)carbamate (72 mg, 0.178 mmol), Pd(dppf)Cl₂·CH₂Cl₂ (14 mg, 0.018 mmol) and K₂CO₃ (61.5 mg, 0.445 mmol) in 1,4-dioxane (900 μL) was stirred at 105° C. for 1 h 20 min. The reaction mixture was diluted with DCM (3 mL) and brine was added (2 mL). The mixture was extracted with further DCM (3×) and the combined organic phases were washed with brine, then filtered and concentrated. The residue was dissolved in 1:1 DCM/MeOH and was filtered through a thiol-silica cartridge to remove Pd. Volatiles were removed and the crude product was purified by FCC (eluting with 0-100% heptane/EtOAc with 3% IPA). The intermediate was treated with 1:1 DCM/TFA (2 mL) at r.t. for 45 min. The volatiles were removed in vacuo. The residue was diluted with MeCN and purified using prep.-LCMS (XBRIDGE® C₁₈ column, eluting with a gradient of MeCN/water containing 0.1% NH₄OH, at flow rate of 60 mL/min.) to afford the title compound as three peaks.

Example 11a. Diastereomer 1. Peak 1. LCMS calc. for C₂₉H₂₄ClF₂N₆S (M+H)⁺ m/z=561.1; found 561.1. This is the potent peak.

Example 11b. Diastereomer 2. Peak 2. LCMS calc. for C₂₉H₂₄ClF₂N₆S (M+H)⁺ m/z=561.1; found 561.1.

Example 11c. Diastereomer 3. Peak 3. LCMS calc. for C₂₉H₂₄ClF₂N₆S (M+H)⁺ m/z=561.1; found 561.1.

The compounds are understood to comprise mixtures of diastereoisomers and/or atropisomers, i.e., 2-amino-(Rₐ)-4-((S)-4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; 2-amino-(Sₐ)-4-((S)-4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; 2-amino-(Rₐ)-4-((R)-4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; and 2-amino-(Sₐ)-4-((R)-4-((R)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile.

The following additional isomers are prepared by an analogous procedure using (S)-3-(1-aminoethyl)-N,N-bis(4-methoxybenzyl)pyridin-2-amine as a starting material in step 3: 2-amino-(Rₐ)-4-((S)-4-((S)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; 2-amino-(Sₐ)-4-((S)-4-((S)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; 2-amino-(Rₐ)-4-((R)-4-((S)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile; and 2-amino-(Sₐ)-4-((R)-4-((S)-1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile.

### Example 12a and 12b. 2-Amino-4-(4-((R)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile

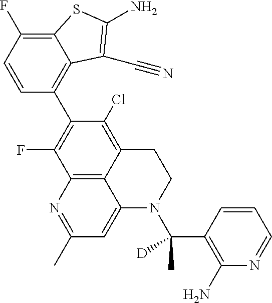 [SMILES: [2H][C@@](C)(c1cccnc1N)N1CCc2c(Cl)c(-c3ccc(F)c4sc(N)c(C#N)c34)c(F)c3nc(C)cc1c23]

### Step 1. (R)-3-(1-Aminoethyl-1-d)-N,N-bis(4-methoxybenzyl)pyridin-2-amine

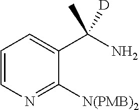

The title compound was prepared according to procedures described in Example 6, Step 1, using sodium borodeuteride as the reducing agent and 1-(2-bis(4-methoxybenzyl)amino)pyridin-3-yl)ethan-1-one as the starting material. LCMS calc. for C₂₃H₂₇DN₃O₂(M+H)⁺ m/z=379.2; found 379.2.

### Step 2. (R)-3-(1-(8-Bromo-7-chloro-9-fluoro-2-methyl-5,6-dihydro-4H-benzo[de][1,6]naphthyridin-4-yl)ethyl-1-d)-N,N-bis(4-methoxybenzyl)pyridin-2-amine

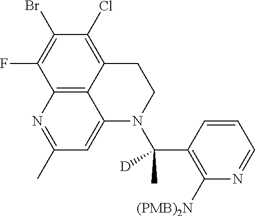

((R)-3-(1-Aminoethyl-1-d)-N-(4-methoxybenzyl)pyridin-2-amine hydrochloride (36.1 mg, 0.123 mmol) to a solution of Intermediate 2 (43 mg, 0.123 mmol) in MeOH (1.2 mL:) followed by AcOH (21 μL, 0.368 mmol), and NaBH₃CN (28.5 mg, 0.453 mmol). The reaction mixture was stirred at r.t. for 3 h. The reaction mixture was diluted with sat. aq. NaHCO₃ and DCM. The aqueous layer was extracted with DCM. The combined organics were dried over Na₂SO₄, filtered and concentrated. The residue was purified by FCC (0-100% EtOAc in hexanes) to provide the title compound as a colorless foam (45 mg, 66%). LCMS calc. for C₃₅H₃₃DBrClFN₄O (M+H)⁺ m/z=676.1, 678.1; found 676.1, 678.1.

### Step 3. (R)-3-(1-(8-Bromo-7-chloro-9-fluoro-2-methyl-5,6-dihydro-4H-benzo[de][1,6]naphthyridin-4-yl)ethyl-1-d)pyridin-2-amine

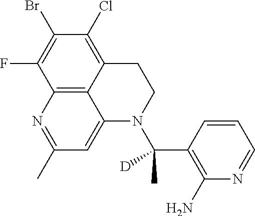 [SMILES: [2H][C@@](C)(c1cccnc1N)N1CCc2c(Cl)c(Br)c(F)c3nc(C)cc1c23]

To (R)-3-(1-(8-bromo-7-chloro-9-fluoro-2-methyl-5,6-dihydro-4H-benzo[de][1,6]naphthyridin-4-yl)ethyl-1-d)-N-(4-methoxybenzyl)pyridin-2-amine (45 mg, 0.081 mmol) was added TFA (250 μL, 3.23 mmol) and the mixture was stirred at 90° C. for 1 h. The volatiles were removed in vacuo. The residue was dissolved in EtOAc and neutralized with sat. NaHCO₃ to pH 7. The resulting precipitate was collected vial filtration and dried under vacuum. The filtrate was purified by FCC (eluting with a gradient of 0-100% EtOAc in hexanes) to provide an additional batch of product as a white solid (33 mg, 94% total yield). LCMS calc. for C₁₉H₁₇DBrClFN₄ (M+H)⁺ m/z=436.0, 438.0; found 436.0, 438.0.

### Step 4. 2-Amino-4-(4-((R)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile

A mixture of (R)-3-(1-(8-bromo-7-chloro-9-fluoro-2-methyl-5,6-dihydro-4H-benzo[de][1,6]naphthyridin-4-yl)ethyl-1-d)pyridin-2-amine (33 mg, 0.076 mmol), tert-butyl (3-cyano-4-(5,5-dimethyl-1,3,2-dioxaborinan-2-yl)-7-fluorobenzo[b]thiophen-2-yl)carbamate (61 mg, 0.151 mmol), Pd(dppf)Cl₂ (12.34 mg, 0.015 mmol) and K₂CO₃ (52.2 mg, 0.378 mmol) in 1,4-dioxane (1 mL) was stirred at 100° C. for 1 h 20 min. The reaction mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 12a. Diastereomer 1. Peak 1. LCMS calc. for C₂₈H₂₁DClF₂N₆S (M+H)⁺ m/z=547.1; found 548.1. This is the potent peak.

Example 12b. Diastereomer 2. Peak 2. LCMS calc. for C₂₈H₂₁DClF₂N₆S (M+H)⁺ m/z=547.1; found 548.1.

The compounds are understood to be atropisomers, i.e., 2-amino-4-((Rₐ)-4-((R)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4—((Sₐ)-4-((R)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile.

The alternative stereoisomers, i.e., 2-amino-4-((Sₐ)-4-((S)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Rₐ)-4-((S)-1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; are prepared by an analogous procedure using (S)-2-methylpropane-2-sulfinamide as a starting material.

### Example 13a and 13b. 2-Amino-4-(4-((R)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile

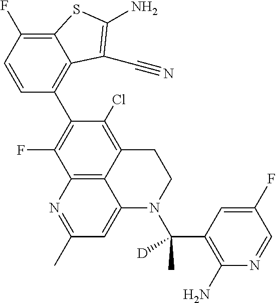 [SMILES: [2H][C@@](C)(c1cc(F)cnc1N)N1CCc2c(Cl)c(-c3ccc(F)c4sc(N)c(C#N)c34)c(F)c3nc(C)cc1c23]

### Step 1. 1-(5-Fluoro-2-((4-methoxybenzyl)amino)pyridin-3-yl)ethan-1-one

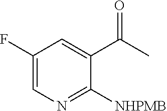

To a solution of 1-(2-chloro-5-fluoropyridin-3-yl)ethan-1-one (501 mg, 2.89 mmol) in 1,4-dioxane (1.5 mL) was added 4-methoxybenzylamine (792 μL, 6.06 mmol). The mixture was stirred at 100° C. overnight. The mixture was cooled to r.t. and DCM was added. The mixture was filtered and the filtrate concentrated. The residue was purified by FCC (0-100% EtOAc in hexanes, 5% DCM additive) to provide the title compound (460 mg, 58%). LCMS calc. for C₁₅H₁₆FN₂O₂(M+H)⁺ m/z=275.2; found 275.2.

### Step 2. 2-Amino-4-(4-((R)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile

The title compound was prepared according to the procedure detailed in Example 6, Steps 1-4, using the product from above as the starting material.

Example 13a. Diastereomer 1. Peak 1. LCMS calc. for C₂₈H₂₀DClF₃N₆S (M+H)⁺ m/z=566.1; found 566.1. This is the more potent diastereoisomer.

Example 13b. Diastereomer 2. Peak 2. LCMS calc. for C₂₈H₂₀DClF₃N₆S (M+H)⁺ m/z=566.1; found 566.1.

The compounds are understood to be atropisomers, i.e., 2-amino-4-((Rₐ)-4-((R)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; 2-amino-4-((Sₐ)-4-((R)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile.

The alternative stereoisomers, i.e., 2-amino-4-((Sₐ)-4-((S)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Rₐ)-4-((S)-1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile; are prepared by an analogous procedure using (S)-2-methylpropane-2-sulfinamide as a starting material.

### Example 14a and 14b. 2-Amino-4-(4-((R)-(2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile

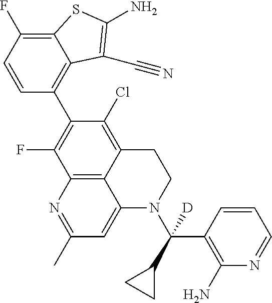

The title compound was prepared according to the general procedures described for Example 13, using (2-chloropyridin-3-yl)(cyclopropyl)methanone as the starting material.

Example 14a. Diastereomer 1. Peak 1. LCMS calc. for C₃₀H₂₃DClF₂N₆S (M+H)⁺ m/z=574.1; found 574.1. This is the more potent diastereoisomer.

Example 14b. Diastereomer 2. Peak 2. LCMS calc. for C₃₀H₂₃DClF₂N₆S (M+H)⁺ m/z=574.1; found 574.1.

The compounds are understood to comprise be atropisomers, i.e., 2-amino-4-((Rₐ)-4-((R)-(2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Sₐ)-4-((R)-(2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile.

The alternative stereoisomers, i.e., 2-amino-4-((Sₐ)-4-((S)-(2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile; and 2-amino-4-((Rₐ)-4-((S)-(2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile; are prepared by an analogous procedure using (S)-2-methylpropane-2-sulfinamide as a starting material.

### Example 15a and 15b. 2-Amino-4-(7-chloro-9-fluoro-2-methyl-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

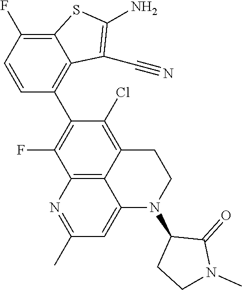 [SMILES: Cc1cc2c3c(c(Cl)c(-c4ccc(F)c5sc(N)c(C#N)c45)c(F)c3n1)CCN2[C@@H]1CCN(C)C1=O]

### Step 1. 2-(7-Bromo-4,6-dichloro-8-fluoro-2-methylquinolin-5-yl)acetaldehyde

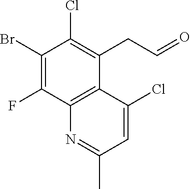 [SMILES: Cc1cc(Cl)c2c(CC=O)c(Cl)c(Br)c(F)c2n1]

To DL-Alanine methyl ester hydrochloride (0.836 g, 5.99 mmol) suspended in DCM (20 mL) was added DIPEA (1.1 mL, 5.99 mmol). The mixture was stirred for 0.5 h before PPTS (1.00 g, 3.99 mmol) and Intermediate 5 (1.84 g, 3.99 mmol) were added. After stirring for another 10 min., sodium perborate tetrahydrate (1.23 g, 7.98 mmol) was added in one portion followed by water (20 mL). The mixture was vigorously stirred for 2 h. Upon completion, the organic layer was separated. The aqueous layer was extracted with DCM (3×). The combined organic layers were dried over Na₂SO₄, filtered and concentrated. The crude product was purified by FCC (0-40% EtOAc/hexanes) to provide the title compound (0.953 g, 68%). LCMS calc. for C₁₂H₈BrCl₂FNO (M+H)⁺: m/z=349.9, 351.9; found 349.8, 351.9.

### Step 2. (R)-3-(8-Bromo-7-chloro-9-fluoro-2-methyl-5,6-dihydro-4H-benzo[de][1,6]naphthyridin-4-yl)-1-methylpyrrolidin-2-one

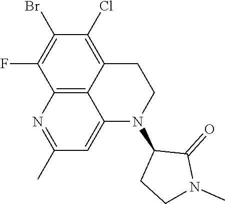 [SMILES: Cc1cc2c3c(c(Cl)c(Br)c(F)c3n1)CCN2[C@@H]1CCN(C)C1=O]

To a solution of 2-(7-bromo-4,6-dichloro-8-fluoro-2-methylquinolin-5-yl)acetaldehyde (903 mg, 2.57 mmol), (R)-3-amino-1-methylpyrrolidin-2-one, HCl salt (550 mg, 3.65 mmol) and PPTS (647 mg, 2.57 mmol) in DCM (12 mL) was added NaBH(OAc)₃ (2181 mg, 10.29 mmol) and the reaction mixture was stirred at r.t. for 1 h. The reaction was quenched with water, extracted with DCM, dried over Na₂SO₄ and concentrated to provide the title compound, which was used in the next step without further purification. LCMS calc. for C₁₇H₁₇BrClFN₃O (M+H)⁺ m/z=414.0, 416.0; found 414.0, 416.0.

### Step 3. tert-Butyl (4-(7-chloro-9-fluoro-2-methyl-4-((R)-1-methyl-2-oxopyrrolidin-3-yl)-5,6-dihydro-4H-benzo[de][1,6]naphthyridin-8-yl)-3-cyano-7-fluorobenzo[b]thiophen-2-yl)carbamate

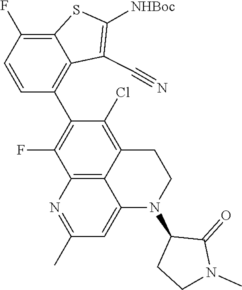 [SMILES: Cc1cc2c3c(c(Cl)c(-c4ccc(F)c5sc(NC(=O)OC(C)(C)C)c(C#N)c45)c(F)c3n1)CCN2[C@@H]1CCN(C)C1=O]

To a mixture of (R)-3-(8-bromo-7-chloro-9-fluoro-2-methyl-5,6-dihydro-4H-benzo[de][1,6]naphthyridin-4-yl)-1-methylpyrrolidin-2-one (500 mg, 1.212 mmol), K₂CO₃ (837 mg, 6.06 mmol), 2-amino-4-(5,5-dimethyl-1,3,2-dioxaborinan-2-yl)-7-fluorobenzo[b]thiophene-3-carbonitrile (737 mg, 2.423 mmol), Pd(dppf)Cl₂ (177 mg, 0.242 mmol) was added 1,4-dioxane (12 mL), and the reaction mixture was placed under a N₂ atmosphere and heated at 100° C. for 2 h. The reaction mixture was cooled to r.t. and diluted with EtOAc, then washed with brine, dried over Na₂SO₄ and concentrated. The crude product was purified by FCC (100% EtOAc) to provide the title compound. LCMS calc. for C₃₁H₂₉ClF₂N₅O₃S (M+H)⁺ m/z=624.2; found 624.2.

### Step 4. 2-Amino-4-(7-chloro-9-fluoro-2-methyl-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

tert-Butyl (4-(7-chloro-9-fluoro-2-methyl-4-((R)-1-methyl-2-oxopyrrolidin-3-yl)-5,6-dihydro-4H-benzo[de][1,6]naphthyridin-8-yl)-3-cyano-7-fluorobenzo[b]thiophen-2-yl)carbamate (1.5 g, 2.403 mmol) was treated with 1:1 DCM/TFA (3 mL) then stirred at r.t. for 0.5 h. The mixture was concentrated. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 15a. Diastereomer 1. Peak 1. LCMS calc. for C₂₆H₂₁ClF₂N₅OS (M+H)⁺ m/z=524.1; found 524.1. This is the potent peak.

Example 15b. Diastereomer 2. Peak 2. LCMS calc. for C₂₆H₂₁ClF₂N₅OS (M+H)⁺ m/z=524.1; found 524.1.

The compounds are understood to comprise atropisomers, i.e., 2-amino-4-(Rₐ)-(7-chloro-9-fluoro-2-methyl-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile.

The alternative stereoisomers, i.e., 2-amino-4-(Sₐ)-(7-chloro-9-fluoro-2-methyl-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; are prepared by an analogous procedure using (S)-3-amino-1-methylpyrrolidin-2-one as a starting material in Step 2.

### Example 16a and 16b. 2-Amino-4-(7-chloro-4-(1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile

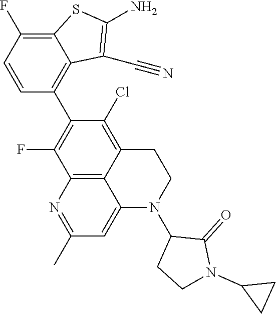 [SMILES: Cc1cc2c3c(c(Cl)c(-c4ccc(F)c5sc(N)c(C#N)c45)c(F)c3n1)CCN2C1CCN(C2CC2)C1=O]

### Step 1. 3-(8-Bromo-7-chloro-9-fluoro-2-methyl-5,6-dihydro-4H-benzo[de][1,6]naphthyridin-4-yl)-1-cyclopropylpyrrolidin-2-one

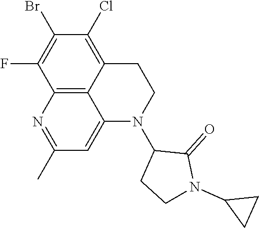 [SMILES: Cc1cc2c3c(c(Cl)c(Br)c(F)c3n1)CCN2C1CCN(C2CC2)C1=O]

To a mixture of 3-amino-1-cyclopropylpyrrolidin-2-one, HCl salt (20 mg, 0.116 mmol) in THF (250 μL) and DCM (250 μL) were added HFIP (83 μL) and DIPEA (24 μL, 0.139 mmol) and the mixture was stirred at r.t. until a homogenous solution was formed (˜20 min.). Intermediate 4 (20 mg, 0.058 mmol), NaBH(OAc)₃ (49 mg, 0.231 mmol) and AcOH (36 μL, 0.463 mmol) were then added, the resulting mixture was stirred at r.t. overnight. The mixture was diluted with EtOAc and aq. NaHCO₃ and the layers were separated. The organic layer was washed with brine, dried over Na₂SO₄ and concentrated. The crude product was purified by FCC (0-100% EtOAc in hexane) to provide the title compound (18 mg, 70%).

### Step 2. 2-Amino-4-(7-chloro-4-(1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile

To the mixture of K₂CO₃ (28.0 mg, 0.203 mmol), Pd(dppf)Cl₂ (6 mg, 8.11 μmol), tert-butyl (3-cyano-4-(5,5-dimethyl-1,3,2-dioxaborinan-2-yl)-7-fluorobenzo[b]thiophen-2-yl)carbamate (33 mg, 0.081 mmol), and 3-(8-bromo-7-chloro-9-fluoro-2-methyl-5,6-dihydro-4H-benzo[de][1,6]naphthyridin-4-yl)-1-cyclopropylpyrrolidin-2-one (18 mg, 0.041 mmol) was added 1,4-dioxane (400 μL) under a N₂ atmosphere. The mixture was heated at 90° C. overnight. The mixture was cooled to r.t., then diluted with EtOAc, filtered and concentrated. The crude product was dissolved in DCM/TFA (1:1, 1 mL), then stirred at r.t. for 30 min. The mixture was concentrated. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 15a. Diastereomer 1. Peak 1. LCMS calc. for C₂₈H₂₃ClF₂N₅OS (M+H)⁺ m/z=550.1; found 550.1. This is the potent peak.

Example 15b. Diastereomer 2. Peak 2. LCMS calc. for C₂₈H₂₃ClF₂N₅OS (M+H)⁺ m/z=550.1; found 550.1.

The compounds are understood to comprise mixtures of diastereoisomers and/or atropisomers, i.e., 2-amino-4-(Rₐ)-(7-chloro-4-((R)-1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and 2-amino-4-(Sₐ)-(7-chloro-4-((S)-1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and 2-amino-4-(Rₐ)-(7-chloro-4-((S)-1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and 2-amino-4-(Sₐ)-(7-chloro-4-((R)-1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Examples 17-24

Compounds in Table 4 were prepared according to the general procedure described for Example 16, using the appropriate amine coupling partner in the reductive amination/cyclization.

TABLE 4

| Ex. | Structure | Name | [M + H] |
|---|---|---|---|
| 17a/b | 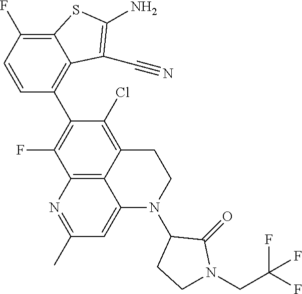 [SMILES: Cc1cc2c3c(c(Cl)c(-c4ccc(F)c5sc(N)c(C#N)c45)c(F)c3n1)CCN2C1CCN(CC(F)(F)F)C1=O] | 2-amino-4-(7-chloro-9-fluoro-2-methyl- 4-(2-oxo-1-(2,2,2-trifluoroethyl) pyrrolidin-3-yl)-5,6-dihydro-4H- benzo[de][1,6]naphthyridin-8-yl)-7- fluorobenzo[b]thiophene-3-carbonitrile | 592.1 |
| 18a/b | 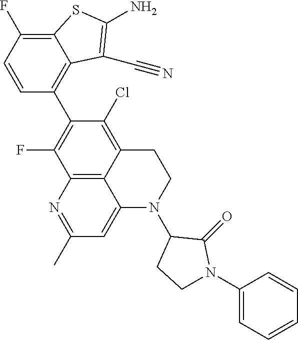 [SMILES: Cc1cc2c3c(c(Cl)c(-c4ccc(F)c5sc(N)c(C#N)c45)c(F)c3n1)CCN2C1CCN(c2ccccc2)C1=O] | 2-amino-4-(7-chloro-9-fluoro-2-methyl- 4-(2-oxo-1-phenylpyrrolidin-3-yl)-5,6- dihydro-4H-benzo[de][1,6]naphthyridin- 8-yl)-7-fluorobenzo[b]thiophene-3- carbonitrile | 586.1 |
| 19a/b | 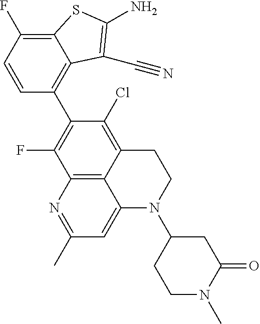 [SMILES: Cc1cc2c3c(c(Cl)c(-c4ccc(F)c5sc(N)c(C#N)c45)c(F)c3n1)CCN2C1CCN(C)C(=O)C1] | 2-amino-4-(7-chloro-9-fluoro-2-methyl- 4-(1-methyl-2-oxopiperidin-4-yl)-5,6- dihydro-4H-benzo[de][1,6]naphthyridin- 8-yl)-7-fluorobenzo[b]thiophene-3- carbonitrile | 538.1 |
| 20a/b/c/d | 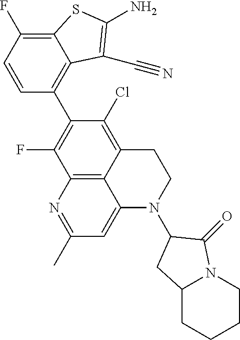 [SMILES: Cc1cc2c3c(c(Cl)c(-c4ccc(F)c5sc(N)c(C#N)c45)c(F)c3n1)CCN2C1CC2CCCCN2C1=O] | 2-amino-4-(7-chloro-9-fluoro-2-methyl- 4-(3-oxooctahydroindolizin-2-yl)-5,6- dihydro-4H-benzo[de][1,6]naphthyridin- 8-yl)-7-fluorobenzo[b]thiophene-3- carbonitrile | 564.1 |
| 21a/b | 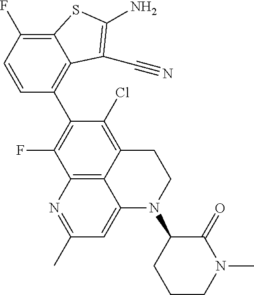 [SMILES: Cc1cc2c3c(c(Cl)c(-c4ccc(F)c5sc(N)c(C#N)c45)c(F)c3n1)CCN2[C@@H]1CCCN(C)C1=O] | 2-amino-4-(7-chloro-9-fluoro-2-methyl- 4-((R)-1-methyl-2-oxopiperidin-3-yl)- 5,6-dihydro-4H- benzo[de][1,6]naphthyridin-8-yl)-7- fluorobenzo[b]thiophene-3-carbonitrile | 538.1 |
| 22a/b | 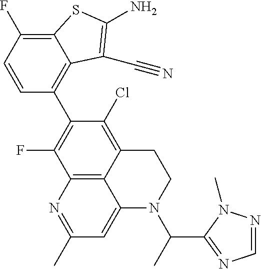 [SMILES: Cc1cc2c3c(c(Cl)c(-c4ccc(F)c5sc(N)c(C#N)c45)c(F)c3n1)CCN2C(C)c1ncnn1C] | 2-amino-4-(7-chloro-9-fluoro-2-methyl- 4-(1-(1-methyl-1H-1,2,4-triazol-5- yl)ethyl)-5,6-dihydro-4H- benzo[de][1,6]naphthyridin-8-yl)-7- fluorobenzo[b]thiophene-3-carbonitrile | 536.1 |
| 23a/b | 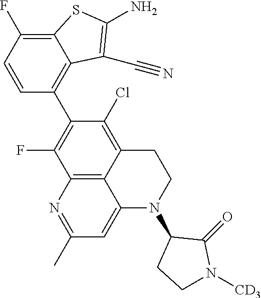 [SMILES: [2H]C([2H])([2H])N1CC[C@@H](N2CCc3c(Cl)c(-c4ccc(F)c5sc(N)c(C#N)c45)c(F)c4nc(C)cc2c34)C1=O] | 2-amino-4-(7-chloro-9-fluoro-2-methyl- 4-((R)-1-(methyl-d3)-2-oxopyrrolidin-3- yl)-5,6-dihydro-4H- benzo[de][1,6]naphthyridin-8-yl)-7- fluorobenzo[b]thiophene-3-carbonitrile | 527.1 |
| 24a/b | 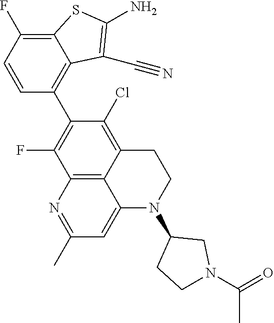 [SMILES: CC(=O)N1CC[C@@H](N2CCc3c(Cl)c(-c4ccc(F)c5sc(N)c(C#N)c45)c(F)c4nc(C)cc2c34)C1] | 4-(4-((R)-1-acetylpyrrolidin-3-yl)-7- chloro-9-fluoro-2-methyl-5,6-dihydro- 4H-benzo[de][1,6]naphthyridin-8-yl)-2- amino-7-fluorobenzo[b]thiophene-3- carbonitrile | 538.1 |

Compounds 17-19, 21 and 22 of Table 4 are understood to comprise mixtures of diastereoisomers and/or atropisomers, i.e.,

### Examples 17a and 17b

2-amino-4-(Rₐ)-(7-chloro-9-fluoro-2-methyl-4-((R)-2-oxo-1-(2,2,2-trifluoroethyl) pyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((S)-2-oxo-1-(2,2,2-trifluoroethyl) pyrrolidin-3-yl)-5,6-dihydro-4H-benzo[de][1,6]naphthyridin-8-yl)-7-fluorobenzo[b]thiophene-3-carbonitrile; and 2-amino-4-(Rₐ)-(7-chloro-9-fluoro-2-methyl-4-((S)-2-oxo-1-(2,2,2-trifluoroethyl) pyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-2-oxo-1-(2,2,2-trifluoroethyl) pyrrolidin-3-yl)-5,6-dihydro-4H-benzo[de][1,6]naphthyridin-8-yl)-7-fluorobenzo[b]thiophene-3-carbonitrile.

### Example 18a and 18b

2-amino-4-(Rₐ)-(7-chloro-9-fluoro-2-methyl-4-((R)-2-oxo-1-phenylpyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((S)-2-oxo-1-phenylpyrrolidin-3-yl)-5,6-dihydro-4H-benzo[de][1,6]naphthyridin-8-yl)-7-fluorobenzo[b]thiophene-3-carbonitrile; and 2-amino-4-(Rₐ)-(7-chloro-9-fluoro-2-methyl-4-((S)-2-oxo-1-phenylpyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-2-oxo-1-phenylpyrrolidin-3-yl)-5,6-dihydro-4H-benzo[de][1,6]naphthyridin-8-yl)-7-fluorobenzo[b]thiophene-3-carbonitrile.

### Example 19a and 19b

2-amino-4-(Rₐ)-(7-chloro-9-fluoro-2-methyl-4-((R)-1-methyl-2-oxopiperidin-4-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((S)-1-methyl-2-oxopiperidin-4-yl)-5,6-dihydro-4H-benzo[de][1,6]naphthyridin-8-yl)-7-fluorobenzo[b]thiophene-3-carbonitrile; and 2-amino-4-(Rₐ)-(7-chloro-9-fluoro-2-methyl-4-((S)-1-methyl-2-oxopiperidin-4-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-1-methyl-2-oxopiperidin-4-yl)-5,6-dihydro-4H-benzo[de][1,6]naphthyridin-8-yl)-7-fluorobenzo[b]thiophene-3-carbonitrile.

### Example 20a, 20b, 20c and 20d

2-amino-4-(Rₐ)-(7-chloro-9-fluoro-2-methyl-4-((2R,8aR)-3-oxooctahydroindolizin-2-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((2S,8aS)-3-oxooctahydroindolizin-2-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((2S,8aR)-3-oxooctahydroindolizin-2-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((2R,8aS)-3-oxooctahydroindolizin-2-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((2R,8aS)-3-oxooctahydroindolizin-2-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((2S,8aR)-3-oxooctahydroindolizin-2-yl)-5,6-dihydro-4H-benzo[de][1,6]naphthyridin-8-yl)-7-fluorobenzo[b]thiophene-3-carbonitrile; and 2-amino-4-(Rₐ)-(7-chloro-9-fluoro-2-methyl-4-((2S,8aS)-3-oxooctahydroindolizin-2-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((2R,8aR)-3-oxooctahydroindolizin-2-yl)-5,6-dihydro-4H-benzo[de][1,6]naphthyridin-8-yl)-7-fluorobenzo[b]thiophene-3-carbonitrile.

### Example 22a and 22b

2-amino-4-(Rₐ)-(7-chloro-9-fluoro-2-methyl-4-((R)-1-(1-methyl-1H-1,2,4-triazol-5-yl)ethyl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((S)-1-(1-methyl-1H-1,2,4-triazol-5-yl)ethyl)-5,6-dihydro-4H-benzo[de][1,6]naphthyridin-8-yl)-7-fluorobenzo[b]thiophene-3-carbonitrile; and 2-amino-4-(Rₐ)-(7-chloro-9-fluoro-2-methyl-4-((S)-1-(1-methyl-1H-1,2,4-triazol-5-yl)ethyl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-1-(1-methyl-1H-1,2,4-triazol-5-yl)ethyl)-5,6-dihydro-4H-benzo[de][1,6]naphthyridin-8-yl)-7-fluorobenzo[b]thiophene-3-carbonitrile.

Compounds 21 and 23-24 of Table 4 are understood to comprise atropisomers, i.e.,

### Example 21a and 21b

2-amino-4-(Rₐ)-(7-chloro-9-fluoro-2-methyl-4-((R)-1-methyl-2-oxopiperidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-1-methyl-2-oxopiperidin-3-yl)-5,6-dihydro-4H-benzo[de][1,6]naphthyridin-8-yl)-7-fluorobenzo[b]thiophene-3-carbonitrile. The alternative isomers, i.e., 2-amino-4-(Rₐ)-(7-chloro-9-fluoro-2-methyl-4-((S)-1-methyl-2-oxopiperidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((S)-1-methyl-2-oxopiperidin-3-yl)-5,6-dihydro-4H-benzo[de][1,6]naphthyridin-8-yl)-7-fluorobenzo[b]thiophene-3-carbonitrile; are prepared by an analogous procedure using (S)-3-amino-1-methylpiperidin-2-one as a starting material.

### Example 23a and 23b

2-amino-4-(Rₐ)-(7-chloro-9-fluoro-2-methyl-4-((R)-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((R)-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile.

The alternative isomers, i.e., 2-amino-4-(Sₐ)-(7-chloro-9-fluoro-2-methyl-4-((S)-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((S)-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; are prepared by an analogous procedure using (S)-3-amino-1-(methyl-d₃)-pyrrolidin-2-one as a starting material.

### Example 24a and 24b

4-(Rₐ)-(4-((R)-1-acetylpyrrolidin-3-yl)-7-chloro-9-fluoro-2-methyl-5,6-dihydro-4H-benzo[de][1,6]naphthyridin-8-yl)-2-amino-7-fluorobenzo[b]thiophene-3-carbonitrile; and 4-(Sₐ)-(4-((R)-1-acetylpyrrolidin-3-yl)-7-chloro-9-fluoro-2-methyl-5,6-dihydro-4H-benzo[de][1,6]naphthyridin-8-yl)-2-amino-7-fluorobenzo[b]thiophene-3-carbonitrile.

The alternative isomers, i.e., 4-(Sₐ)-(4-((S)-1-acetylpyrrolidin-3-yl)-7-chloro-9-fluoro-2-methyl-5,6-dihydro-4H-benzo[de][1,6]naphthyridin-8-yl)-2-amino-7-fluorobenzo[b]thiophene-3-carbonitrile; 4-(Rₐ)-(4-((S)-1-acetylpyrrolidin-3-yl)-7-chloro-9-fluoro-2-methyl-5,6-dihydro-4H-benzo[de][1,6]naphthyridin-8-yl)-2-amino-7-fluorobenzo[b]thiophene-3-carbonitrile; and are prepared by an analogous procedure using (S)-1-(3-aminopyrrolidin-1-yl)ethanone as a starting material.

### Example 25a, 25b, 25c and 25d. 2-Amino-4-(7-chloro-9-fluoro-2-methyl-4-(1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile

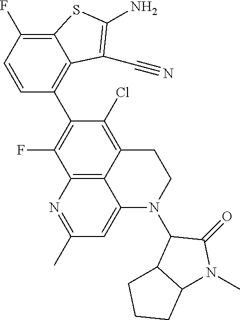 [SMILES: Cc1cc2c3c(c(Cl)c(-c4ccc(F)c5sc(N)c(C#N)c45)c(F)c3n1)CCN2C1C(=O)N(C)C2CCCC21]

### Step 1. 1-Methylhexahydrocyclopenta[b]pyrrol-2 (1H)-one

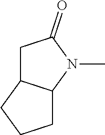

To a solution of hexahydrocyclopenta[b]pyrrol-2 (1H)-one (500 mg, 4.0 mmol) in THF (10 mL) was added NaH (60% in mineral oil, 192 mg, 4.8 mmol) and the mixture was stirred at r.t. for 5 min., then methyl iodide (300 μL, 4.8 mmol) was added and the mixture was stirred at r.t. for 2 h. The reaction was quenched with sat. aq. NaHCO₃ then extracted with EtOAc. The organic layer was washed with brine, dried over MgSO₄ and concentrated. The crude product was purified by FCC (0-100% EtOAc in hexanes) to provide the title compound. LCMS calc. for C₈H₁₄NO (M+H)⁺ m/z=140.1; found 140.1.

### Step 2. 3-Azido-1-methylhexahydrocyclopenta[b]pyrrol-2 (1H)-one

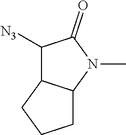

To a solution of 1-methylhexahydrocyclopenta[b]pyrrol-2 (1H)-one (120 mg, 0.86 mmol) in THF (4 mL) at −78° C. was added LDA (1 M in THF/hexanes, 900 μL, 900 mmol) and the reaction mixture was stirred at this temperature for 30 min. A solution of 2,4,6-triisopropylbenzenesulfonyl azide (10% in toluene, 3.23 mL, 0.902 mmol) was then added and the mixture was allowed to warm to r.t. over the course of 4 h. The reaction was quenched with sat. aq. NH₄Cl, then extracted with EtOAc. The organic layer was washed with brine, dried over MgSO₄ and concentrated. The crude product was purified by FCC (0-100% EtOAc in hexanes) to provide the title compound (147 mg, 95%). LCMS calc. for C₃H₁₃N₄O (M+H)⁺ m/z=181.1; found 181.1.

### Step 3. 3-Amino-1-methylhexahydrocyclopenta[b]pyrrol-2 (1H)-one

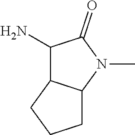

To a solution of 3-azido-1-methylhexahydrocyclopenta[b]pyrrol-2 (1H)-one (147 mg, 0.82 mmol) in EtOAc (5 mL) was added palladium on carbon (10% by weight, 43 mg, 0.04 mmol) and the reaction flask was placed under a hydrogen atmosphere from a balloon, then stirred at r.t. for 24 h. The reaction mixture was filtered through a pad of diatomaceous earth and concentrated. The crude product was used in the next step without further purification. LCMS calc. for C₈H₁₅N₂₀ (M+H)⁺ m/z=155.1; found 155.1.

### Step 4. 2-Amino-4-(7-chloro-9-fluoro-2-methyl-4-(1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile

The title compound was prepared according to the general procedures set forth in Example 16 using the amine from Step 3 in the reductive amination.

Example 25a. Diastereomer 1. Peak 1. LCMS calc. for C₂₉H₂₅ClF₂N₅OS (M+H)⁺ m/z=564.1; found 564.1. This is the potent peak.

Example 25b. Diastereomer 2. Peak 2 LCMS calc. for C₂₉H₂₅ClF₂N₅OS (M+H)⁺ m/z=564.1; found 564.1.

Example 25c. Diastereomer 3. Peak 3. LCMS calc. for C₂₉H₂₅ClF₂N₅OS (M+H)⁺ m/z=564.1; found 564.1.

Example 25d. Diastereomer 4. Peak 4. LCMS calc. for C₂₉H₂₅ClF₂N₅OS (M+H)⁺ m/z=564.1; found 564.1.

The compounds are understood to comprise mixtures of diastereoisomers and/or atropisomers, i.e., 2-amino-4-(Rₐ)-(7-chloro-9-fluoro-2-methyl-4-((3R,3aR,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3S,3aS,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3S,3aR,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3R,3aS,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3R,3aS,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3S,3aR,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3S,3aS,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3R,3aR,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile.

Alternative isomers are prepared using the appropriate isomer of hexahydrocyclopenta[b]pyrrol-2 (1H)-one as the starting material, i.e., 2-amino-4-(Rₐ)-(7-chloro-9-fluoro-2-methyl-4-((3R,3aR,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3S,3aS,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3S,3aR,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3R,3aS,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3R,3aR,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3S,3aS,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((3S,3aR,6aS)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((3R,3aS,6aR)-1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile.

### Examples 26-27

Compounds in Table 5 were prepared according to the general procedure outlined in Example 25 using the appropriate starting material.

TABLE 5

| Example | Structure | Name | [M + H]+ |
|---|---|---|---|
| 26a/b | 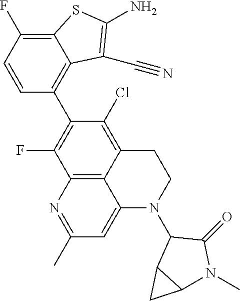 [SMILES: Cc1cc2c3c(c(Cl)c(-c4ccc(F)c5sc(N)c(C#N)c45)c(F)c3n1)CCN2C1C(=O)N(C)C2CC21] | 2-amino-4-(7-chloro-9-fluoro-2- methyl-4-(2-methyl-3-oxo-2- azabicyclo[3.1.0]hexan-4-yl)-5,6- dihydro-4H- benzo[de][1,6]naphthyridin-8-yl)-7- fluorobenzo[b]thiophene-3- carbonitrile | 536.1 |
| 27a/b/c/d | 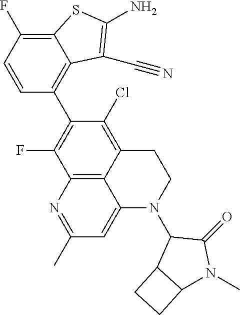 [SMILES: Cc1cc2c3c(c(Cl)c(-c4ccc(F)c5sc(N)c(C#N)c45)c(F)c3n1)CCN2C1C(=O)N(C)C2CCC21] | 2-amino-4-(7-chloro-9-fluoro-2- methyl-4-(2-methyl-3-oxo-2- azabicyclo[3.2.0]heptan-4-yl)-5,6- dihydro-4H- benzo[de][1,6]naphthyridin-8-yl)-7- fluorobenzo[b]thiophene-3- carbonitrile | 550.1 |

The compounds of Table 5 are understood to comprise mixtures of diastereoisomers and/or atropisomers, i.e.,

### Example 26a and 26b

2-amino-4-(Rₐ)-(7-chloro-9-fluoro-2-methyl-4-((1S,4R,5R)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1R,4S,5S)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1R,4R,5S)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1 S,4S,5R)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1 S,4S,5R)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1R,4R,5S)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1R,4S,5S)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1 S,4R,5R)-2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile.

### Examples 27a, 27b, 27c, and 27d

2-amino-4-(Rₐ)-(7-chloro-9-fluoro-2-methyl-4-((1S,4R,5R)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1R,4S,5S)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1R,4R,5S)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1 S,4R,5R)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1 S,4S,5R)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Sₐ)-(7-chloro-9-fluoro-2-methyl-4-((1R,4R,5S)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; 2-amino-4-(Rₐ)-(7-chloro-9-fluoro-2-methyl-4-((1R,4S,5S)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-9-fluoro-2-methyl-4-((1 S,4S,5S)-2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile.

### Example 28. 2-Amino-4-(7-chloro-2-(3-(dimethylamino)-3-methylazetidin-1-yl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

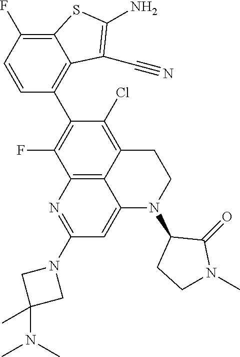

### Step 1. (R)-3-(8-Bromo-2,7-dichloro-9-fluoro-5,6-dihydro-4H-benzo[de][1,6]naphthyridin-4-yl)-1-methylpyrrolidin-2-one

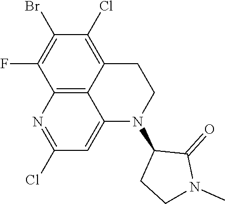 [SMILES: CN1CC[C@@H](N2CCc3c(Cl)c(Br)c(F)c4nc(Cl)cc2c34)C1=O]

To the mixture of (R)-3-amino-1-methylpyrrolidin-2-one, HCl salt (896 mg, 5.95 mmol) in THF (11 mL) and DCE (11 mL), HFIP (3.73 mL) and DIPEA (2.006 mL, 11.49 mmol) were added. The mixture was stirred at r.t. until a homogenous solution formed (˜20 min.), followed by the addition of Intermediate 4 (1100 mg, 2.87 mmol), NaBH(OAc)₃ (2434 mg, 11.5 mmol) and AcOH (1.77 mL, 22.97 mmol). The resulting mixture was stirred at r.t. overnight. The mixture was diluted with EtOAc and the layers were separated. The aqueous layer was extracted with additional EtOAc and the combined organics were washed with brine, dried over Na₂SO₄ and concentrated. The crude product was used in the next step without further purification. LCMS calc. for C₁₆H₁₄BrCl₂FN₃O (M+H)⁺ m/z=431.9, 433.9; found 431.9, 433.9.

### Step 2. (R)-3-(8-Bromo-7-chloro-2-(3-(dimethylamino)-3-methylazetidin-1-yl)-9-fluoro-5,6-dihydro-4H-benzo[de][1,6]naphthyridin-4-yl)-1-methylpyrrolidin-2-one

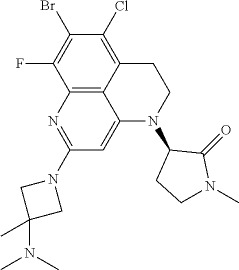

(R)-3-(8-bromo-2,7-dichloro-9-fluoro-5,6-dihydro-4H-benzo[de][1,6]naphthyridin-4-yl)-1-methylpyrrolidin-2-one (22 mg, 0.051 mmol), N,N,3-trimethylazetidin-3-amine dihydrochloride (47.5 mg, 0.254 mmol) and DIPEA (106 μL, 0.610 mmol) were dissolved in NMP (250 μL) under N₂. The mixture was heated at 120° C. overnight, then allowed to cool to r.t. The mixture was diluted with MeOH and purified by supercritical fluid chromatography (2-pic column, eluting with a gradient of CO₂/MeOH 12-22% over 10 min. at 140 mL/min.) to provide the title compound (24 mg, 93%). LCMS calc. for C₂₂H₂₇BrClFN₅O (M+H)⁺ m/z=510.1, 512.1; found 510.1, 512.1.

### Step 3. 2-Amino-4-(7-chloro-2-(3-(dimethylamino)-3-methylazetidin-1-yl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

To a mixture of (R)-3-(8-bromo-7-chloro-2-(3-(dimethylamino)-3-methylazetidin-1-yl)-9-fluoro-5,6-dihydro-4H-benzo[de][1,6]naphthyridin-4-yl)-1-methylpyrrolidin-2-one (22 mg, 0.043 mmol), tert-butyl (3-cyano-4-(5,5-dimethyl-1,3,2-dioxaborinan-2-yl)-7-fluorobenzo[b]thiophen-2-yl)carbamate (34.8 mg, 0.086 mmol), Pd(dppf)Cl₂ (7.7 mg, 10.77 μmol), and K₂CO₃ (18 mg, 0.129 mmol) was added 1,4-dioxane (500 μL) under N₂. The reaction mixture was heated at 100° C. for 2 h. The reaction was quenched with water and extracted with EtOAc. The organic layer was washed with brine, dried over Na₂SO₄ and concentrated. The residue was treated with 1:1 DCM/TFA (1 mL) at r.t. for 30 min., then concentrated. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound. LCMS calc. for C₃₁H₃₁ClF₂N₇OS (M+H)⁺ m/z=622.2; found 622.2.

The compound is is understood to comprise a mixture of atropisomers, i.e., 2-amino-4-(Rₐ)-(7-chloro-2-(3-(dimethylamino)-3-methylazetidin-1-yl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-2-(3-(dimethylamino)-3-methylazetidin-1-yl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile.

The alternative isomers are prepared by an analogous route using (S)-3-amino-1-methylpyrrolidin-2-one as a starting material: 2-amino-4-(Rₐ)-(7-chloro-2-(3-(dimethylamino)-3-methylazetidin-1-yl)-9-fluoro-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; and 2-amino-4-(Sₐ)-(7-chloro-2-(3-(dimethylamino)-3-methylazetidin-1-yl)-9-fluoro-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile.

### Example 29a and 29b. 8-(2-Amino-3-cyano-7-fluorobenzo[b]thiophen-4-yl)-4-(1-(2-aminopyridin-3-yl)ethyl)-7-chloro-9-fluoro-N-methyl-5,6-dihydro-4H-benzo[de][1,6]naphthyridine-2-carboxamide

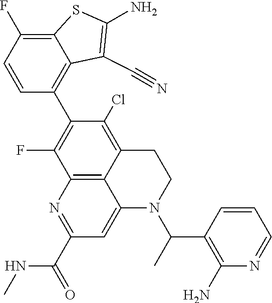 [SMILES: CNC(=O)c1cc2c3c(c(Cl)c(-c4ccc(F)c5sc(N)c(C#N)c45)c(F)c3n1)CCN2C(C)c1cccnc1N]

### Step 1. 2,4,7-Tribromo-5-(bromomethyl)-6-chloro-8-fluoroquinoline

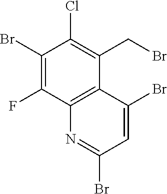 [SMILES: Fc1c(Br)c(Cl)c(CBr)c2c(Br)cc(Br)nc12]

A solution of 7-bromo-6-chloro-8-fluoro-5-(hydroxymethyl)quinoline-2,4-diol (Intermediate 2, Step 6, 2.4 g, 7.46 mmol) in phosphorus oxybromide (7.6 mL, 75 mmol) was heated at 120° C. for 24 h, cooled to 0° C., then quenched carefully with ice water. The resulting mixture was stirred at r.t. for 15 min., then DCM/EtOAc (2:1, ˜100 mL) was added and the layers separated. The organic layer was washed with brine, dried over MgSO₄ and concentrated. The crude product was triturated with EtOAc/hexanes (1:1) to provide the title compound, which was used in the next step without further purification (3.3 g, 88%). LCMS calc. for C₁₀H₄Br₄ClFN (M+H)⁺ m/z=509.7, 511.7, 513.7; found 509.7, 511.7, 513.7.

### Step 2. 2-(2,4,7-Tribromo-6-chloro-8-fluoroquinolin-5-yl)acetonitrile

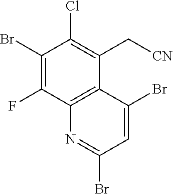 [SMILES: N#CCc1c(Cl)c(Br)c(F)c2nc(Br)cc(Br)c12]

To a solution of 2,4,7-tribromo-5-(bromomethyl)-6-chloro-8-fluoroquinoline (500 mg, 0.98 mmol) in MeCN (4 mL) was added NaCN (57 mg, 1.2 mmol) and the reaction mixture was stirred at r.t. for 3 h, at which point an additional portion of NaCN was added and heating was continued for 3 h at 90° C. The mixture was cooled to r.t. and concentrated. The residue was dissolved in EtOAc, then washed with brine, dried over MgSO₄ and concentrated. The crude product was purified by FCC (0-60% EtOAc in hexanes) to provide the title compound (302 mg, 68%). LCMS calc. for C₁₁H₄Br₃ClFN₂ (M+H)⁺ m/z=456.8, 458.8; found 456.8, 458.8.

### Step 3. 2-(2,4,7-Tribromo-6-chloro-8-fluoroquinolin-5-yl)acetaldehyde

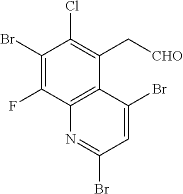 [SMILES: O=CCc1c(Cl)c(Br)c(F)c2nc(Br)cc(Br)c12]

To a solution of 2-(2,4,7-tribromo-6-chloro-8-fluoroquinolin-5-yl)acetonitrile (294 mg, 0.64 mmol) in DCM (4 mL) at −78° C. was added DIBAL-H (1.0 M in DCM, 710 μL, 0.71 mmol) dropwise and the mixture was stirred at this temperature for 3 h. The mixture was was then warmed up to 0° C. and quenched by the addition of isopropyl alcohol. The mixture was diluted with EtOAc and washed with brine. The organic layer was dried over MgSO₄ and concentrated. The crude product was purified by FCC (30-45% EtOAc in hexanes) to provide the title compound. LCMS calc. for C₁₁H₅Br₃ClFNO (M+H)⁺ m/z=459.8, 461.8; found 459.8, 461.8.

### Step 4. 3-(1-(2,8-Dibromo-7-chloro-9-fluoro-5,6-dihydro-4H-benzo[de][1,6]naphthyridin-4-yl)ethyl)-N,N-bis(4-methoxybenzyl)pyridin-2-amine

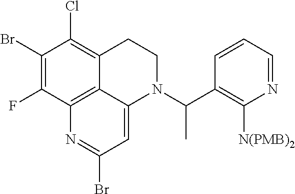

To a mixture of 2-(2,4,7-tribromo-6-chloro-8-fluoroquinolin-5-yl)acetaldehyde (35 mg, 0.08 mmol), 3-(1-aminoethyl)-N,N-bis(4-methoxybenzyl)pyridin-2-amine (36 mg, 0.094 mmol) in THF (400 μL) were added NaBH(OAc)₃ (24 mg, 0.11 mmol) and AcOH (11 μL, 0.15 mmol). The reaction mixture was stirred at r.t. for 30 min., then the reaction was quenched with sat. aq. NaHCO₃. The mixture was extracted with EtOAc and the organic layer was washed with brine, dried over MgSO₄ and concentrated. The crude product was purified by FCC (0-100% EtOAc in hexanes) to provide the title compound (26 mg, 46%). LCMS calc. for C₃₄H₃₁Br₂ClFN₄O₂(M+H)⁺ m/z=741.0, 743.0; found 741.0, 743.0.

### Step 5. 4-(1-(2-(Bis(4-methoxybenzyl)amino)pyridin-3-yl)ethyl)-8-bromo-7-chloro-9-fluoro-N-methyl-5,6-dihydro-4H-benzo[de][1,6]naphthyridine-2-carboxamide

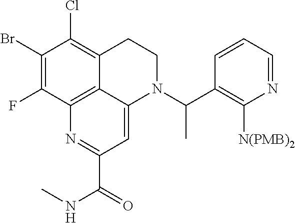

To a mixture of Pd(dppf)Cl₂ (3.6 mg, 4.9 μmol), methylamine hydrochloride (2.0 mg, 0.03 mmol) and 3-(1-(2,8-dibromo-7-chloro-9-fluoro-5,6-dihydro-4H-benzo[de][1,6]naphthyridin-4-yl)ethyl)-N,N-bis(4-methoxybenzyl)pyridin-2-amine (18 mg, 0.024 mmol) was added 1,4-dioxane (500 μL) and the reaction vessel was placed under an atmosphere of CO from a balloon, then stirred at 80° C. overnight. The reaction was quenched with sat. aq. NaHCO₃, then extracted with EtOAc. The organic layer was washed with brine, dried over MgSO₄ and concentrated. The crude product was purified by FCC (0-100% EtOAc in hexanes) to provide the title compound (9 mg, 51%). LCMS calc. for C₃₆H₃₅BrClFN₅O₃ (M+H)⁺ m/z=718.1, 720.1; found 718.1, 720.1

### Step 6. 4-(1-(2-Aminopyridin-3-yl)ethyl)-8-bromo-7-chloro-9-fluoro-N-methyl-5,6-dihydro-4H-benzo[de][1,6]naphthyridine-2-carboxamide

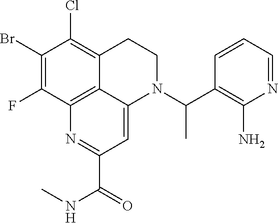 [SMILES: CNC(=O)c1cc2c3c(c(Cl)c(Br)c(F)c3n1)CCN2C(C)c1cccnc1N]

A solution of 4-(1-(2-(bis(4-methoxybenzyl)amino)pyridin-3-yl)ethyl)-8-bromo-7-chloro-9-fluoro-N-methyl-5,6-dihydro-4H-benzo[de][1,6]naphthyridine-2-carboxamide (9 mg, 0.012 mmol) in TFA (400 μL) was stirred at 90° C. 1 h, then concentrated. The residue was dissolved in a EtOAc/sat. aq. NaHCO₃ mixture and the layers separated. The organic layer was washed with brine, dried over MgSO₄ and concentrated. The crude product was used in the next step without further purification. LCMS calc. for C₂₀H₁₉BrClFN₅O (M+H)⁺ m/z=478.0, 480.0; found 478.0, 480.0.

### Step 7. 8-(2-Amino-3-cyano-7-fluorobenzo[b]thiophen-4-yl)-4-(1-(2-aminopyridin-3-yl)ethyl)-7-chloro-9-fluoro-N-methyl-5,6-dihydro-4H-benzo[de][1,6]naphthyridine-2-carboxamide

To a mixture of K₂CO₃ (15 mg, 0.11 mmol), Pd(dppf)Cl₂ (3 mg, 0.43 μmol), 4-(1-(2-aminopyridin-3-yl)ethyl)-8-bromo-7-chloro-9-fluoro-N-methyl-5,6-dihydro-4H-benzo[de][1,6]naphthyridine-2-carboxamide (10 mg, 0.022 mmol) and tert-butyl (3-cyano-4-(5,5-dimethyl-1,3,2-dioxaborinan-2-yl)-7-fluorobenzo[b]thiophen-2-yl)carbamate (17 mg, 0.043 mmol) was added 1,4-dioxane (400 μL). The reaction flask was put under a N₂ atmosphere, then stirred at 100° C. for 1 h. After cooling to r.t., the mixture was diluted with EtOAc and filtered. The filtrate was concentrated, then redissolved in 1:1 DCM/TFA (1 mL) and stirred at r.t. for 30 min. The mixture was concentrated. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 29a. Diastereomer 1. Peak 1. LCMS calc. for C₂₉H₂₃ClF₂N₇OS (M+H)⁺ m/z=590.1; found 590.1. This is the potent peak.

Example 29b. Diastereomer 2. Peak 2. LCMS calc. for C₂₉H₂₃ClF₂N₇OS (M+H)⁺ m/z=590.1; found 590.1.

The compounds are understood to comprise mixtures of stereoisomers, i.e., 8-(2-amino-3-cyano-7-fluorobenzo[b]thiophen-4-yl)-4-((R)-1-(2-aminopyridin-3-yl)ethyl)-7-chloro-9-fluoro-N-methyl-5,6-dihydro-4H—(Rₐ)-benzo[de][1,6]naphthyridine-2-carboxamide; and 8-(2-amino-3-cyano-7-fluorobenzo[b]thiophen-4-yl)-4-((S)-1-(2-aminopyridin-3-yl)ethyl)-7-chloro-9-fluoro-N-methyl-5,6-dihydro-4H—(Sₐ)-benzo[de][1,6]naphthyridine-2-carboxamide; and 8-(2-amino-3-cyano-7-fluorobenzo[b]thiophen-4-yl)-4-((S)-1-(2-aminopyridin-3-yl)ethyl)-7-chloro-9-fluoro-N-methyl-5,6-dihydro-4H—(Rₐ)-benzo[de][1,6]naphthyridine-2-carboxamide; and 8-(2-amino-3-cyano-7-fluorobenzo[b]thiophen-4-yl)-4-((R)-1-(2-aminopyridin-3-yl)ethyl)-7-chloro-9-fluoro-N-methyl-5,6-dihydro-4H—(Sₐ)-benzo[de][1,6]naphthyridine-2-carboxamide.

### Example 30a and 30b. 2-Amino-4-(4-((R)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile

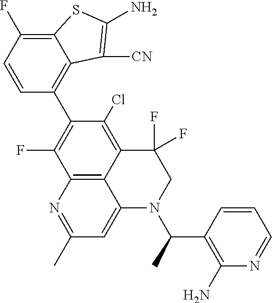 [SMILES: Cc1cc2c3c(c(Cl)c(-c4ccc(F)c5sc(N)c(C#N)c45)c(F)c3n1)C(F)(F)CN2[C@H](C)c1cccnc1N]

### Step 1. 2-(7-Bromo-4,6-dichloro-8-fluoro-2-methylquinolin-5-yl)-2,2-difluoroacetaldehyde

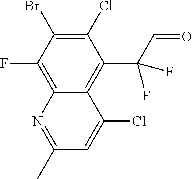 [SMILES: Cc1cc(Cl)c2c(C(F)(F)C=O)c(Cl)c(Br)c(F)c2n1]

To a vial containing Intermediate 2 (0.141 g, 0.40 mmol), N-fluorobenzenesulfonimide (0.507 g, 1.61 mmol) and PPTS (0.101 g, 0.40 mmol) were added anhydrous THF (1.4 mL) and (R)-2-(diphenyl(trimethylsilyl)oxy)methyl)pyrrolidine (0.026 g, 0.080 mmol). The mixture was stirred under N₂ for 16 h. Upon completion, sat. aq. NaHCO₃ was added. The mixture was stirred for 0.5 h and then extracted with EtOAc (3×). The combined organic layers were dried over Na₂SO₄, filtered and concentrated. The crude product was purified by FCC (0-40% EtOAc/hexanes) to provide the title compound. LCMS calc. for C₁₂H₅BrCl₂F3NO (M+H)⁺: m/z=385.9, 387.9; found 385.9, 387.9.

### Step 2. (R)-3-(1-(8-Bromo-7-chloro-6,6,9-trifluoro-2-methyl-5,6-dihydro-4H-benzo[de][1,6]naphthyridin-4-yl)ethyl)-N,N-bis(4-methoxybenzyl)pyridin-2-amine

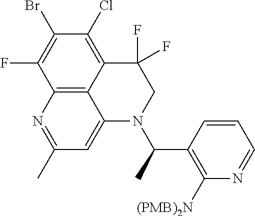

To a DCM solution (0.5 mL) containing 2-(7-bromo-4,6-dichloro-8-fluoro-2-methylquinolin-5-yl)-2,2-difluoroacetaldehyde (0.020 g, 0.052 mmol) was added PPTS (0.013 g, 0.052 mmol) and (R)-3-(1-aminoethyl)-N,N-bis(4-methoxybenzyl)pyridin-2-amine (0.020 g, 0.052 mmol). The mixture was stirred for 0.5 h at r.t. before NaBH₃CN (0.015 g, 0.25 mmol) was added. The mixture was stirred for 0.5 h before being quenched by addition of water. The mixture was extracted with DCM (3×). The combined organic layers were dried over Na₂SO₄, filtered and concentrated. The crude product was purified by FCC (0-50% EtOAc/hexanes) to provide the title compound. LCMS calc. for C₃₅H₃₂BrClF₃N₄O₂ (M+H)⁺: m/z=711.1, 713.1; found 711.0, 713.0.

### Step 3. (R)-3-(1-(8-Bromo-7-chloro-6,6,9-trifluoro-2-methyl-5,6-dihydro-4H-benzo[de][1,6]naphthyridin-4-yl)ethyl)pyridin-2-amine

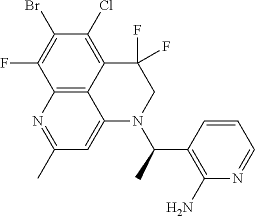 [SMILES: Cc1cc2c3c(c(Cl)c(Br)c(F)c3n1)C(F)(F)CN2[C@H](C)c1cccnc1N]

A TFA (0.5 mL) solution containing (R)-3-(1-(8-bromo-7-chloro-6,6,9-trifluoro-2-methyl-5,6-dihydro-4H-benzo[de][1,6]naphthyridin-4-yl)ethyl)-N,N-bis(4-methoxybenzyl)pyridin-2-amine (0.030 g, 0.042 mmol) was heated at 90° C. for 1 h. After cooling to r.t., the residue was dissolved in DCM and treated with sat. aq. NaHCO₃. The mixture was extracted with DCM (3×). The combined organic layers were dried over Na₂SO₄, filtered and concentrated to afford the title compound. LCMS calc. for C₁₉H₁₆BrClF₃N₄ (M+H)⁺: m/z=471.0, 473.0; found 471.0, 473.0.

### Step 4. 2-Amino-4-(4-((R)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile

To a vial containing (R)-3-(1-(8-bromo-7-chloro-6,6,9-trifluoro-2-methyl-5,6-dihydro-4H-benzo[de][1,6]naphthyridin-4-yl)ethyl)pyridin-2-amine (0.020 g, 0.042 mmol), tert-butyl (3-cyano-4-(5,5-dimethyl-1,3,2-dioxaborinan-2-yl)-7-fluorobenzo[b]thiophen-2-yl)carbamate (0.034 g, 0.085 mmol), Pd(dppf)Cl₂·CH₂Cl₂ (0.007 g, 0.0085 mmol) and K₂CO₃ (0.029 g, 0.21 mmol) was added 1,4-dioxane (0.660 mL). The reaction was heated at 95° C. under N₂ for 1 h. After cooling to r.t., the mixture was diluted with EtOAc and washed with brine. The organic phase was dried over Na₂SO₄, filtered and concentrated. To the residue dissolved in DCM (0.5 mL) was added TFA (0.5 mL). The mixture was stirred for 0.5 h. Upon completion, volatiles were removed under reduced pressure and the residue was dissolved in MeCN (4 mL) and water (1 mL) and purified by preparative LCMS (XBridge C18 column, eluting with a gradient of MeCN/water containing 0.1% TFA, at flow rate of 60 mL/min.) to afford the title compound.

Example 30a. Diastereomer 1. Peak 1. LCMS calc. for C₂₈H₂₀ClF₄N₆S (M+H)⁺: m/z=583.1; found 583.0 This is the potent peak.

Example 30b. Diastereomer 2. Peak 2 LCMS calc. for C₂₈H₂₀ClF₄N₆S (M+H)⁺: m/z=583.1; found 583.0.

The compounds are understood to comprise atropisomers, i.e., 2-amino-4-(Rₐ)-(4-((R)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile; and 2-amino-4-(Sₐ)-(4-((R)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile.

The alternative isomers, i.e., 2-amino-4-(Sₐ)-(4-((S)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile; and 2-amino-4-(Rₐ)-(4-((S)-1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile; are prepared by an analogous procedure using (S)-3-(1-aminoethyl)-N,N-bis(4-methoxybenzyl)pyridin-2-amine as a starting material in Step 2.

### Example 31a and 31b. (3R)-3-(8-(2-Amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one

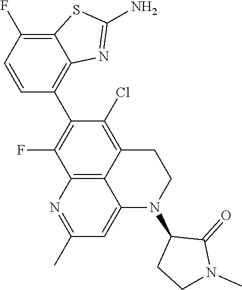 [SMILES: Cc1cc2c3c(c(Cl)c(-c4ccc(F)c5sc(N)nc45)c(F)c3n1)CCN2[C@@H]1CCN(C)C1=O]

A solution of (R)-3-(8-bromo-7-chloro-9-fluoro-2-methyl-5,6-dihydro-4H-benzo[de][1,6]naphthyridin-4-yl)-1-methylpyrrolidin-2-one (Example 15, Step 2, 15 mg, 0.036 mmol), (2-((tert-butoxycarbonyl)amino)-7-fluorobenzo[d]thiazol-4-yl)boronic acid (23 mg, 0.073 mmol), Pd(PPh₃)₄, (8 mg, 7.3 μmol), and K₃PO₄ (23 mg, 0.11 mmol) in 1,4-dioxane (500 μL) and water (100 μL) was sparged with N₂ and heated at 110° C. for 18 h. After cooling to r.t., the mixture was diluted with EtOAc, filtered, washed with brine, dried over Na₂SO₄, filtered and concentrated. The crude residue was dissolved in DCM (1 mL) and TFA (1 mL) was added. After stirring at r.t. for 1 h, the mixture was concentrated and purified by prep. HPLC (XBRIDGE® C₁₈ column, eluting with a gradient of MeCN/water containing 0.1% NH₄OH, at flow rate of 60 mL/min.) to afford the title compound as two peaks.

Example 31a. Diastereomer 1. Peak 1. LCMS calc. for C₂₄H₂₁ClF₂N₅OS (M+H)⁺ m/z=500.1; found 500.1. This is the potent peak.

Example 31b. Diastereomer 2. Peak 2. LCMS calc. for C₂₄H₂₁ClF₂N₅OS (M+H)⁺ m/z=500.1; found 500.1.

The compounds are understood to comprise atropisomers, i.e., (3R)-3-(8-(Rₐ)-(2-amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one; and (3R)-3-(8-(Sₐ)-(2-amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one.

The alternative isomers, i.e., (3S)-3-(8-(Rₐ)-(2-amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one; and (3S)-3-(8-(Sₐ)-(2-amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one; are prepared by an analogous procedure using (S)-3-(8-bromo-7-chloro-9-fluoro-2-methyl-5,6-dihydro-4H-benzo[de][1,6]naphthyridin-4-yl)-1-methylpyrrolidin-2-one as starting material.

### Example 32a and 32b. 8-(2-Amino-3-cyano-7-fluorobenzo[b]thiophen-4-yl)-7-chloro-9-fluoro-N-methyl-4-((R)-1-methyl-2-oxopyrrolidin-3-yl)-5,6-dihydro-4H-benzo[de][1,6]naphthyridine-2-carboxamide

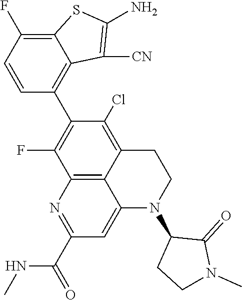 [SMILES: CNC(=O)c1cc2c3c(c(Cl)c(-c4ccc(F)c5sc(N)c(C#N)c45)c(F)c3n1)CCN2[C@@H]1CCN(C)C1=O]

### Step 1. (R)-3-(2,8-Dibromo-7-chloro-9-fluoro-5,6-dihydro-4H-benzo[de][1,6]naphthyridin-4-yl)-1-methylpyrrolidin-2-one

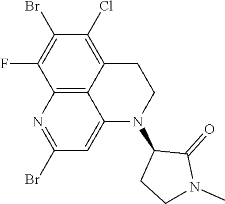 [SMILES: CN1CC[C@@H](N2CCc3c(Cl)c(Br)c(F)c4nc(Br)cc2c34)C1=O]

(R)-3-Amino-1-methylpyrrolidin-2-one 4-methylbenzenesulfonate (560 mg, 1.956 mmol) was dissolved in THF (4 mL), DCM (4 mL), and HFIP (1.3 mL) and DIPEA (388 μL, 2.22 mmol) was added, the mixture was stirred at r.t. for 5 min. 2-(2,7-dibromo-6-chloro-8-fluoro-4-(methylthio)quinolin-5-yl)acetaldehyde (Example 29, Step 3, 380 mg, 0.889 mmol), NaBH(OAc)₃ (754 mg, 3.56 mmol) and AcOH (548 μL, 7.11 mmol) were then added, the resulting mixture was stirred at r.t. overnight. The reaction was quenched with aq. NaHCO₃, and extracted with EtOAc (3×). The combined organic phases were washed with brine, dried over MgSO₄ and concentrated. The crude product was purified by FCC (0-100% EtOAc in hexanes, then 100% EtOAc) to provide the title compound. LCMS calc. for C₁₆H₁₄Br₂ClFN₃O (M+H)⁺ m/z=475.9, 477.9, 479.9; found 475.9, 477.9, 479.9.

### Step 2. (R)-8-Bromo-7-chloro-9-fluoro-N-methyl-4-(1-methyl-2-oxopyrrolidin-3-yl)-5,6-dihydro-4H-benzo[de][1,6]naphthyridine-2-carboxamide

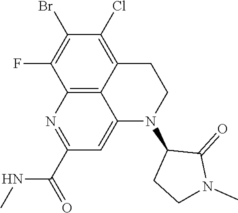

A solution of (R)-3-(2,8-dibromo-7-chloro-9-fluoro-5,6-dihydro-4H-benzo[de][1,6]naphthyridin-4-yl)-1-methylpyrrolidin-2-one (104 mg, 0.22 mmol), (dppf)PdCl₂ (18 mg, 0.022 mmol) and K₂CO₃ (150 mg, 1.09 mmol) in 1,4-dioxane (2.2 mL) was degassed with CO from a balloon for 10 min., then methylamine hydrochloride (44 mg, 0.65 mmol) was added and the mixture was placed under a CO atmosphere and stirred at 70° C. for 72 h. The reaction was quenched with sat. aq. NH₄Cl and extracted with EtOAc. The organic layer was washed with 1 N HCl, water and brine, dried over MgSO₄ and concentrated. The crude product was purified by FCC (20% EtOAc in hexanes) to provide the title compound. LCMS calc. for C₁₃H₁₃BrClFN₄O₂(M+H)⁺ m/z=455.0, 457.0; found 455.0, 457.0.

### Step 3. 8-(2-Amino-3-cyano-7-fluorobenzo[b]thiophen-4-yl)-7-chloro-9-fluoro-N-methyl-4-((R)-1-methyl-2-oxopyrrolidin-3-yl)-5,6-dihydro-4H-benzo[de][1,6]naphthyridine-2-carboxamide

A mixture of (R)-8-bromo-7-chloro-9-fluoro-N-methyl-4-(1-methyl-2-oxopyrrolidin-3-yl)-5,6-dihydro-4H-benzo[de][1,6]naphthyridine-2-carboxamide (23 mg, 0.050 mmol), tert-butyl (3-cyano-4-(5,5-dimethyl-1,3,2-dioxaborinan-2-yl)-7-fluorobenzo[b]thiophen-2-yl)carbamate (41 mg, 0.10 mmol), (dppf)PdCl₂ (8 mg, 10 μmol) and K₂CO₃ (35 mg, 0.25 mmol) in 1,4-dioxane (1 mL) was stirred at 90° C. for 1 h 20 min. The mixture was filtered and concentrated. The crude product was treated with 1:1 DCM/TFA (1 mL) at r.t. for 50 min. The volatiles were removed in vacuo. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 32a. Diastereomer 1. Peak 1. LCMS calc. for C₂₇H₂₂ClF₂N₆O₂S (M+H)⁺ m/z=567.1; found 567.1. This is the potent peak.

Example 32b. Diastereomer 2. Peak 2. LCMS calc. for C₂₇H₂₂ClF₂N₆O₂S (M+H)⁺ m/z=567.1; found 567.1.

The compounds are understood to comprise atropisomers, i.e., 8-(Rₐ)-(2-amino-3-cyano-7-fluorobenzo[b]thiophen-4-yl)-7-chloro-9-fluoro-N-methyl-4-((R)-1-methyl-2-oxopyrrolidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-N-methyl-4-((R)-1-methyl-2-oxopyrrolidin-3-yl)-5,6-dihydro-4H-benzo[de][1,6]naphthyridine-2-carboxamide.

The alternative isomers, i.e., 8-(Rₐ)-(2-amino-3-cyano-7-fluorobenzo[b]thiophen-4-yl)-7-chloro-9-fluoro-N-methyl-4-((S)-1-methyl-2-oxopyrrolidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-N-methyl-4-((S)-1-methyl-2-oxopyrrolidin-3-yl)-5,6-dihydro-4H-benzo[de][1,6]naphthyridine-2-carboxamide; are prepared by an analogous procedure using (S)-3-amino-1-methylpyrrolidin-2-one 4-methylbenzenesulfonate as starting material.

### Example 33a, 33b, 33c and 33d. 2-Amino-4-(7-chloro-9-fluoro-2-(1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

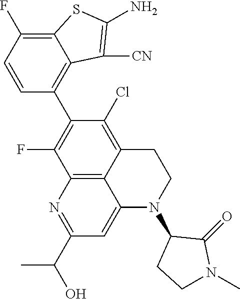 [SMILES: CC(O)c1cc2c3c(c(Cl)c(-c4ccc(F)c5sc(N)c(C#N)c45)c(F)c3n1)CCN2[C@@H]1CCN(C)C1=O]

### Step 1. (R)-3-(8-Bromo-7-chloro-9-fluoro-2-(prop-1-en-2-yl)-5,6-dihydro-4H-benzo[de][1,6]naphthyridin-4-yl)-1-methylpyrrolidin-2-one

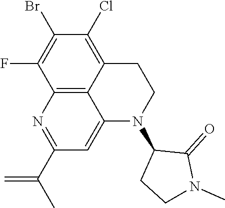 [SMILES: C=C(C)c1cc2c3c(c(Cl)c(Br)c(F)c3n1)CCN2[C@@H]1CCN(C)C1=O]

In a 2 dram vial, Intermediate 10 (255 mg, 0.49 mmol), 4,4,5,5-tetramethyl-2-(prop-1-en-2-yl)-1,3,2-dioxaborolane (137 μl, 0.729 mmol), Pd(Ph₃P)₄ (56 mg, 0.049 mmol), and Cs₂CO₃ (475 mg, 1.46 mmol) were dissolved in 1,4-dioxane/water (5:1, 2.4 mL total volume) under N₂. The mixture was heated at 90° C. for 30 min., then quenched with water and extracted with DCM. The organic layer was dried over MgSO₄ and concentrated. The crude product was purified by FCC (0-100% DCM in heptane) to provide the title compound (208 mg, 97%). LCMS calc. for C₁₉H₁₉BrClFN₃O (M+H)⁺ m/z=438.0, 440.0; found 438.0, 440.0.

### Step 2. (4-(7-Chloro-9-fluoro-4-((R)-1-methyl-2-oxopyrrolidin-3-yl)-2-(prop-1-en-2-yl)-5,6-dihydro-4H-benzo[de][1,6]naphthyridin-8-yl)-3-cyano-7-fluorobenzo[b]thiophen-2-yl)carbamate

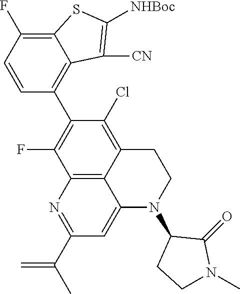 [SMILES: C=C(C)c1cc2c3c(c(Cl)c(-c4ccc(F)c5sc(NC(=O)OC(C)(C)C)c(C#N)c45)c(F)c3n1)CCN2[C@@H]1CCN(C)C1=O]

To a mixture of (R)-3-(8-bromo-7-chloro-9-fluoro-2-(prop-1-en-2-yl)-5,6-dihydro-4H-benzo[de][1,6]naphthyridin-4-yl)-1-methylpyrrolidin-2-one (40 mg, 0.091 mmol), tert-butyl (3-cyano-4-(5,5-dimethyl-1,3,2-dioxaborinan-2-yl)-7-fluorobenzo[b]thiophen-2-yl)carbamate (74 mg, 0.182 mmol), dichloro[bis(diphenylphosphinophenyl)ether]palladium(II) (16 mg, 0.023 mmol), and K₂CO₃ (38 mg, 0.274 mmol) was added 1,4-dioxane (500 μL) under N₂. The mixture was heated at 105° C. for 90 min. The mixture was then diluted with water/DCM and the phases were separated. The organic layer was dried over MgSO₄ and concentrated. The crude product was purified by FCC (0-20% MeOH in DCM) to provide the title compound. LCMS calc. for C₃₃H₃₁ClF₂N₅O₃S (M+H)⁺ m/z=650.2; found 650.2.

### Step 3. 2-Amino-4-(7-chloro-9-fluoro-2-(1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

tert-Butyl (4-(7-chloro-9-fluoro-4-((R)-1-methyl-2-oxopyrrolidin-3-yl)-2-(prop-1-en-2-yl)-5,6-dihydro-4H-benzo[de][1,6]naphthyridin-8-yl)-3-cyano-7-fluorobenzo[b]thiophen-2-yl)carbamate (50 mg, 0.077 mmol) was dissolved in THF (2.5 mL) open to air. Osmium tetroxide (4% w/v in water, 0.241 mL, 0.031 mmol) was added and the mixture was stirred at r.t. for 3 min. NaIO₄ (66 mg, 0.308 mmol) was added to the mixture at once as a solid powder. The mixture was stirred at r.t. for 45 min., then diluted with water. The mixture was extracted with DCM. The organic layer was dried over MgSO₄ and concentrated.

To the crude product were added a 1:1 mixture of THF/MeOH (2.5 mL total volume) and NaBH₄ (20 mg, 0.53 mmol) and the mixture was stirred at r.t. for 30 min. The reaction was quenched with water and extracted with DCM. The organic layer was dried over MgSO₄ and concentrated. The crude product was taken up in 1 mL TFA, stirred 5 min. and concentrated. The crude product was diluted with MeCN and purified by prep. HPLC (XBRIDGE® C₁₈ column, eluting with a gradient of MeCN/water containing 0.1% NH₄OH, at flow rate of 60 mL/min.) to afford the title compound as four peaks.

Example 33a. Diastereomer 1. Peak 1. LCMS calc. for C₂₇H₂₃ClF₂N₅O₂S (M+H)⁺ m/z=554.1; found 554.1.

Example 33b. Diastereomer 2. Peak 2. LCMS calc. for C₂₇H₂₃ClF₂N₅O₂S (M+H)⁺ m/z=554.1; found 554.1. This is the potent peak.

Example 33c. Diastereomer 3. Peak 3. LCMS calc. for C₂₇H₂₃ClF₂N₅O₂S (M+H)⁺ m/z=554.1; found 554.1. This is the potent peak.

Example 33d. Diastereomer 4. Peak 4. LCMS calc. for C₂₇H₂₃ClF₂N₅O₂S (M+H)⁺ m/z=554.1; found 554.1.

The compounds are understood to comprise diastereoisomers and atropisomers, i.e., (Rₐ)-2-amino-4-(7-chloro-9-fluoro-2-((R)-1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-((R)-1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-((S)-1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-((S)-1-hydroxyethyl)-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-((S)-1-hydroxyethyl)-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-((S)-1-hydroxyethyl)-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-((R)-1-hydroxyethyl)-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-((R)-1-hydroxyethyl)-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; are prepared by an analogous procedure using the (S)-enantiomer of Intermediate 10 as starting material.

### Example 34a and 34b. 2-Amino-4-(7-chloro-9-fluoro-2-methoxy-4-((R)-1-methyl-2-oxopyrrolidin-3-yl)-5,6-dihydro-4H-benzo[de][1,6]naphthyridin-8-yl)-7-fluorobenzo [b]thiophene-3-carbonitrile

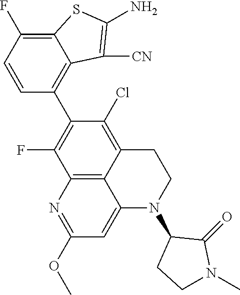 [SMILES: COc1cc2c3c(c(Cl)c(-c4ccc(F)c5sc(N)c(C#N)c45)c(F)c3n1)CCN2[C@@H]1CCN(C)C1=O]

### Step 1. (R)-3-(8-Bromo-7-chloro-9-fluoro-2-methoxy-5,6-dihydro-4H-benzo[de][1,6]naphthyridin-4-yl)-1-methylpyrrolidin-2-one

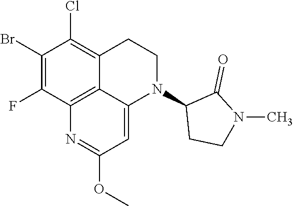 [SMILES: COc1cc2c3c(c(Cl)c(Br)c(F)c3n1)CCN2[C@@H]1CCN(C)C1=O]

In a 1 dram vial, Intermediate 10 (30 mg, 0.057 mmol), copper(I) iodide (3 mg, 0.014 mmol), Cs₂CO₃ (48 mg, 0.15 mmol), MeOH (4 μL) and 3,4,7,8-tetramethyl-1,10-phenanthroline (7 mg, 0.03 mmol) were dissolved in toluene (400 μL) under N₂. The mixture was heated at 80° C. with rapid stirring for 10 min., then cooled to r.t. The mixture was diluted with MeCN and filtered. The filtrate was concentrated. The residue was partitioned between water and EtOAc. The organic layer was separated, washed with water and brine, dried over MgSO₄ and concentrated. The crude product was used in the next step without further purification. LCMS calc. for C₁₇H₁₇BrClFN₃O₂(M+H)⁺ m/z=428.0, 430.0; found 428.0, 430.0.

### Step 2. 2-Amino-4-(7-chloro-9-fluoro-2-methoxy-4-((R)-1-methyl-2-oxopyrrolidin-3-yl)-5,6-dihydro-4H-benzo[de][1,6]naphthyridin-8-yl)-7-fluorobenzo [b]thiophene-3-carbonitrile

In a 1 dram vial, (R)-3-(8-bromo-7-chloro-9-fluoro-2-methoxy-5,6-dihydro-4H-benzo[de][1,6]naphthyridin-4-yl)-1-methylpyrrolidin-2-one (15 mg, 0.035 mmol), tert-butyl (3-cyano-4-(5,5-dimethyl-1,3,2-dioxaborinan-2-yl)-7-fluorobenzo[b]thiophen-2-yl)carbamate (28 mg, 0.070 mmol), dichloro[bis(diphenylphosphinophenyl)ether]palladium(II) (6. mg, 8.75 μmol), and K₂CO₃ (14 mg, 0.105 mmol) were dissolved in 1,4-dioxane (250 μL) under N₂. The mixture was heated at 100° C. for 1 h. The reaction was quenched with water and the mixture was extracted with DCM. The organic layer was washed with water and brine, dried over MgSO₄ and concentrated. The crude product was dissolved in DCM (1 mL) and TFA (1 mL) was added. After stirring at r.t. for 30 min., the mixture was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 34a. Diastereomer 1. Peak 1. LCMS calc. for C₂₆H₂₁ClF₂N₅O₂S (M+H)⁺ m/z=540.1; found 540.1. This is the potent peak.

Example 34b. Diastereomer 2. Peak 2. LCMS calc. for C₂₆H₂₁ClF₂N₅O₂S (M+H)⁺ m/z=540.1; found 540.1.

The compounds are understood to comprise atropisomers, i.e., (Rₐ)-2-amino-4-(7-chloro-9-fluoro-2-methoxy-4-((R)-1-methyl-2-oxopyrrolidin-3-yl)-5,6-dihydro-4H-benzo[de][1,6]naphthyridin-8-yl)-7-fluorobenzo [b]thiophene-3-carbonitrile; and (Sₐ)-2-amino-4-(7-chloro-9-fluoro-2-methoxy-4-((R)-1-methyl-2-oxopyrrolidin-3-yl)-5,6-dihydro-4H-benzo[de][1,6]naphthyridin-8-yl)-7-fluorobenzo [b]thiophene-3-carbonitrile.

The alternative isomers, i.e., (Rₐ)-2-amino-4-(7-chloro-9-fluoro-2-methoxy-4-((S)-1-methyl-2-oxopyrrolidin-3-yl)-5,6-dihydro-4H-benzo[de][1,6]naphthyridin-8-yl)-7-fluorobenzo [b]thiophene-3-carbonitrile; and (Sₐ)-2-amino-4-(7-chloro-9-fluoro-2-methoxy-4-((S)-1-methyl-2-oxopyrrolidin-3-yl)-5,6-dihydro-4H-benzo[de][1,6]naphthyridin-8-yl)-7-fluorobenzo [b]thiophene-3-carbonitrile are prepared by an analogous procedure using the (S)-enantiomer of Intermediate 10 as starting material.

### Example 35a and 35b. 8-(2-amino-3-cyano-7-fluorobenzo[b]thiophen-4-yl)-7-chloro-9-fluoro-4-((R)-1-methyl-2-oxopyrrolidin-3-yl)-N-(1-methylazetidin-3-yl)-5,6-dihydro-4H-benzo[de][1,6]naphthyridine-2-carboxamide

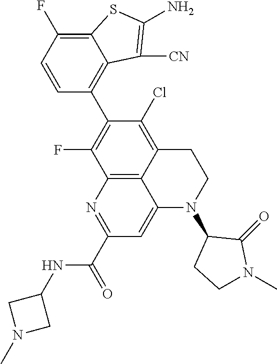

### Step 1. Methyl (R)-8-bromo-7-chloro-9-fluoro-4-(1-methyl-2-oxopyrrolidin-3-yl)-5,6-dihydro-4H-benzo[de][1,6]naphthyridine-2-carboxylate

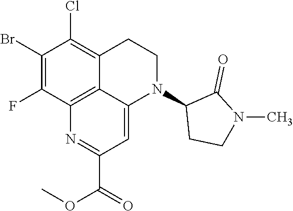 [SMILES: COC(=O)c1cc2c3c(c(Cl)c(Br)c(F)c3n1)CCN2[C@@H]1CCN(C)C1=O]

Under an atmosphere of argon, a solution of Intermediate 10 (550 mg, 1.05 mmol), and (dppf)PdCl₂ (86 mg, 0.11 mmol) in 1,4-dioxane (5.2 mL)/MeOH (5.2 mL) was degassed for 10 min. with CO gas. Then TEA (731 μL, 5.24 mmol) was added and the solution was stirred at 60° C. under a CO atmosphere for 18 h. The mixture was concentrated and the crude product was purified by FCC (20% EtOAc in hexanes) to afford the title compound (471 mg, 98%). LCMS calc. for C₁₃H₁₇BrClFN₃O₃(M+H)⁺ m/z=456.0, 458.0; found 456.0, 458.0.

### Step 2. (R)-8-Bromo-7-chloro-9-fluoro-4-(1-methyl-2-oxopyrrolidin-3-yl)-5,6-dihydro-4H-benzo[de][1,6]naphthyridine-2-carboxylic acid

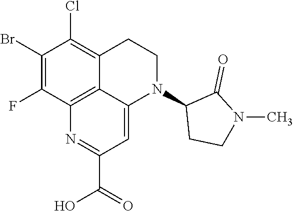 [SMILES: CN1CC[C@@H](N2CCc3c(Cl)c(Br)c(F)c4nc(C(=O)O)cc2c34)C1=O]

To solution of methyl (R)-8-bromo-7-chloro-9-fluoro-4-(1-methyl-2-oxopyrrolidin-3-yl)-5,6-dihydro-4H-benzo[de][1,6]naphthyridine-2-carboxylate (810 mg, 1.77 mmol) in THF (6 mL) was added NaOH (1.0 N in water, 3.5 mL, 3.5 mmol) at 50° C. After stirring for 1 h at this temperature, the mixture was diluted with water and EtOAc. To the mixture was added 1 M HCl to adjust the pH to 7. The mixture was diluted with EtOAc. The organic layer was separated, washed with brine, dried with Na₂SO₄ and concentrated. The crude product was used in the next step without further purification. LCMS calc. for C₂₁1H₂₃BrClFN₅O₂(M+H)⁺ m/z=442.0, 444.0; found 442.0, 444.0.

### Step 3. (R)-8-Bromo-7-chloro-9-fluoro-4-(1-methyl-2-oxopyrrolidin-3-yl)-N-(1-methylazetidin-3-yl)-5,6-dihydro-4H-benzo[de][1,6]naphthyridine-2-carboxamide

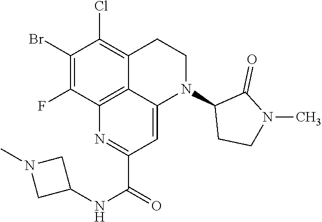

To a solution of (R)-8-bromo-7-chloro-9-fluoro-4-(1-methyl-2-oxopyrrolidin-3-yl)-5,6-dihydro-4H-benzo[de][1,6]naphthyridine-2-carboxylic acid (21 mg, 0.047 mmol) in DMF (160 μL) were added HATU (22 mg, 0.057 mmol), 1-methylazetidine-3-amine (5 mg, 0.062 mmol) and DIEA (30 μL, 0.171 mmol). The resulting mixture was stirred at r.t. for 1 h. The mixture was diluted with MeCN and purified using prep.-LCMS (XBRIDGE® C₁₈ column, eluting with a gradient of MeCN/water containing 0.1% TFA, at flow rate of 60 mL/min.) to afford the title compound as the corresponding TFA salt. LCMS calc. for C₁₇H₁₅BrClFN₃O₃ (M+H)⁺ m/z=510.1, 512.1; found 510.1, 512.1.

### Step 4. 8-(2-Amino-3-cyano-7-fluorobenzo[b]thiophen-4-yl)-7-chloro-9-fluoro-4-((R)-1-methyl-2-oxopyrrolidin-3-yl)-N-(1-methylazetidin-3-yl)-5,6-dihydro-4H-benzo[de][1,6]naphthyridine-2-carboxamide

A mixture of (R)-8-bromo-7-chloro-9-fluoro-4-(1-methyl-2-oxopyrrolidin-3-yl)-N-(1-methylazetidin-3-yl)-5,6-dihydro-4H-benzo[de][1,6]naphthyridine-2-carboxamide (23 mg, 0.045 mmol), tert-butyl (3-cyano-4-(5,5-dimethyl-1,3,2-dioxaborinan-2-yl)-7-fluorobenzo[b]thiophen-2-yl)carbamate (36 mg, 0.09 mmol), (dppf)PdCl₂ (7 mg, 9 μmol) and K₂CO₃ (31 mg, 0.225 mmol) in 1,4-dioxane (1 mL) was stirred at 100° C. for 80 min. The mixture was filtered and concentrated. The crude residue was treated with 1:1 DCM/TFA (1 mL) at r.t. for 50 min. The volatiles were removed in vacuo. The mixture was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 35a. Diastereomer 1. Peak 1. LCMS calc. for C₃₀H₂₇ClF₂N₇O₂S (M+H)⁺ m/z=622.1; found 622.1. This is the potent peak.

Example 35b. Diastereomer 2. Peak 2. LCMS calc. for C₃₀H₂₇ClF₂N₇O₂S (M+H)⁺ m/z=622.1; found 622.1.

The compounds are understood to comprise atropisomers, i.e., 8-(Rₐ)-(2-amino-3-cyano-7-fluorobenzo[b]thiophen-4-yl)-7-chloro-9-fluoro-4-((R)-1-methyl-2-oxopyrrolidin-3-yl)-N-(1-methylazetidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-4-((R)-1-methyl-2-oxopyrrolidin-3-yl)-N-(1-methylazetidin-3-yl)-5,6-dihydro-4H-benzo[de][1,6]naphthyridine-2-carboxamide.

The alternative isomers, i.e., 8-(Rₐ)-(2-amino-3-cyano-7-fluorobenzo[b]thiophen-4-yl)-7-chloro-9-fluoro-4-((S)-1-methyl-2-oxopyrrolidin-3-yl)-N-(1-methylazetidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-4-((S)-1-methyl-2-oxopyrrolidin-3-yl)-N-(1-methylazetidin-3-yl)-5,6-dihydro-4H-benzo[de][1,6]naphthyridine-2-carboxamide; are prepared by an analogous procedure using the (S)-enantiomer of Intermediate 10 as starting material.

### Example 36a and 36b. 8-(2-Amino-3-cyano-7-fluorobenzo[b]thiophen-4-yl)-7-chloro-9-fluoro-N-(2-hydroxy-2-methylpropyl)-4-((R)-1-methyl-2-oxopyrrolidin-3-yl)-5,6-dihydro-4H-benzo[de][1,6]naphthyridine-2-carboxamide

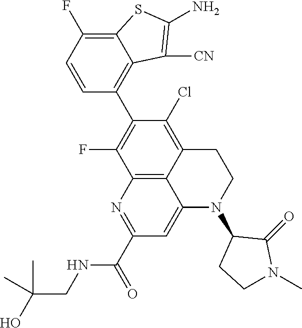 [SMILES: CN1CC[C@@H](N2CCc3c(Cl)c(-c4ccc(F)c5sc(N)c(C#N)c45)c(F)c4nc(C(=O)NCC(C)(C)O)cc2c34)C1=O]

The title compound was prepared according to the general procedure described in Example 35, utilizing 1-amino-2-methylpropan-2-ol as the coupling partner in Step 3. The final product was isolated as two peaks.

Example 36a. Diastereomer 1. Peak 1. LCMS calc. for C₃₀H₂₈ClF₂N₆O₃S (M+H)⁺ m/z=625.1; found 625.1. This is the potent peak.

Example 36b. Diastereomer 2. Peak 2. LCMS calc. for C₃₀H₂₈ClF₂N₆O₃S (M+H)⁺ m/z=625.1; found 625.1.

The compounds are understood to comprise atropisomers, i.e., 8-(Rₐ)-(2-amino-3-cyano-7-fluorobenzo[b]thiophen-4-yl)-7-chloro-9-fluoro-N-(2-hydroxy-2-methylpropyl)-4-((R)-1-methyl-2-oxopyrrolidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-N-(2-hydroxy-2-methylpropyl)-4-((R)-1-methyl-2-oxopyrrolidin-3-yl)-5,6-dihydro-4H-benzo[de][1,6]naphthyridine-2-carboxamide.

The alternative isomers, i.e., 8-(Rₐ)-(2-amino-3-cyano-7-fluorobenzo[b]thiophen-4-yl)-7-chloro-9-fluoro-N-(2-hydroxy-2-methylpropyl)-4-((S)-1-methyl-2-oxopyrrolidin-3-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-N-(2-hydroxy-2-methylpropyl)-4-((S)-1-methyl-2-oxopyrrolidin-3-yl)-5,6-dihydro-4H-benzo[de][1,6]naphthyridine-2-carboxamide; are prepared by an analogous procedure using the (S)-enantiomer of Intermediate 10 as starting material.

### Example 37. N-(8-(2-Amino-3-cyano-7-fluorobenzo[b]thiophen-4-yl)-7-chloro-9-fluoro-4-((R)-1-methyl-2-oxopyrrolidin-3-yl)-5,6-dihydro-4H-benzo[de][1,6]naphthyridin-2-yl)acetamide

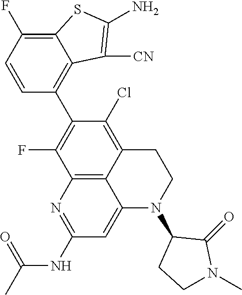 [SMILES: CC(=O)Nc1cc2c3c(c(Cl)c(-c4ccc(F)c5sc(N)c(C#N)c45)c(F)c3n1)CCN2[C@@H]1CCN(C)C1=O]

### Step 1. (R)-3-(8-Bromo-7-chloro-2-((diphenylmethylene)amino)-9-fluoro-5,6-dihydro-4H-benzo[de][1,6]naphthyridin-4-yl)-1-methylpyrrolidin-2-one

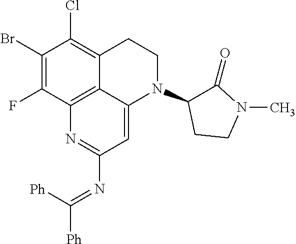 [SMILES: CN1CC[C@@H](N2CCc3c(Cl)c(Br)c(F)c4nc(N=C(c5ccccc5)c5ccccc5)cc2c34)C1=O]

To a vial containing Intermediate 10 (133 mg, 0.253 mmol), diphenylmethanimine (85 μL, 0.51 mmol), Pd₂(dba)₃ (62 mg, 0.060 mmol), Xantphos (70 mg, 0.12 mmol) and Cs₂CO₃ (165 mg, 0.51 mmol) was added 1,4-dioxane (1.3 mL). The mixture was sparged with N₂ and heated at 80° C. for 3 h. The mixture was allowed to cool to r.t. and filtered through a plug of diatomaceous earth. The filtrate was concentrated and purified by FCC (0-100% EtOAc in heptane) to provide the title compound (118 mg, 81%). LCMS calc. for C₂₉H₂₄BrClFN₄O (M+H)⁺ m/z=577.1, 579.1; found 577.1, 579.1.

### Step 2. (R)-3-(2-Amino-8-bromo-7-chloro-9-fluoro-5,6-dihydro-4H-benzo[de][1,6]naphthyridin-4-yl)-1-methylpyrrolidin-2-one

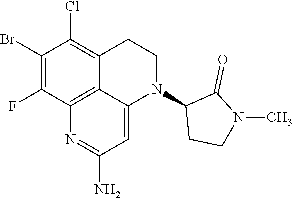 [SMILES: CN1CC[C@@H](N2CCc3c(Cl)c(Br)c(F)c4nc(N)cc2c34)C1=O]

To a solution of (R)-3-(8-bromo-7-chloro-2-((diphenylmethylene)amino)-9-fluoro-5,6-dihydro-4H-benzo[de][1,6]naphthyridin-4-yl)-1-methylpyrrolidin-2-one (66 mg, 0.11 mmol) in THF (2 mL) was added HCl (1 N, 341 μL, 341 mmol) and the mixture was stirred at 105° C. for 90 min., then concentrated and purified by FCC (0-100% acetone in heptane) to provide the title compound. LCMS calc. for C₁₆H₁₆BrClFN₄O (M+H)⁺ m/z=413.0, 415.0; found 413.0, 415.0.

### Step 3. (R)—N-(8-Bromo-7-chloro-9-fluoro-4-(1-methyl-2-oxopyrrolidin-3-yl)-5,6-dihydro-4H-benzo[de][1,6]naphthyridin-2-yl)acetamide

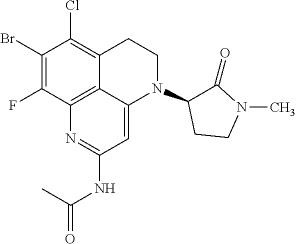 [SMILES: CC(=O)Nc1cc2c3c(c(Cl)c(Br)c(F)c3n1)CCN2[C@@H]1CCN(C)C1=O]

In a 2 dram vial, (R)-3-(2-amino-8-bromo-7-chloro-9-fluoro-5,6-dihydro-4H-benzo[de][1,6]naphthyridin-4-yl)-1-methylpyrrolidin-2-one (40 mg, 0.097 mmol), DIPEA (84 μL, 0.483 mmol) and AcCl (155 μL, 0.155 mmol) were dissolved in DCM (1 mL) under N₂. The mixture was stirred at r.t. for 30 min., then quenched with water and extracted with DCM. The organic layer was washed with brine, dried over MgSO₄ and concentrated. The crude product was used in the next step without further purification. LCMS calc. for C₁₃H₁₃BrClFN₄O₂(M+H)⁺ m/z=455.0, 457.0; found 455.0, 457.0.

### Step 4. N-(8-(2-Amino-3-cyano-7-fluorobenzo[b]thiophen-4-yl)-7-chloro-9-fluoro-4-((R)-1-methyl-2-oxopyrrolidin-3-yl)-5,6-dihydro-4H-benzo[de][1,6]naphthyridin-2-yl)acetamide

In a 1 dram vial, (R)—N-(8-bromo-7-chloro-9-fluoro-4-(1-methyl-2-oxopyrrolidin-3-yl)-5,6-dihydro-4H-benzo[de][1,6]naphthyridin-2-yl)acetamide (11 mg, 0.025 mmol), tert-butyl (3-cyano-4-(5,5-dimethyl-1,3,2-dioxaborinan-2-yl)-7-fluorobenzo[b]thiophen-2-yl)carbamate (20 mg, 0.05 mmol), dichloro[bis(diphenylphosphinophenyl)ether]palladium(II) (4 mg, 6.2 μmol), and K₂CO₃ (10 mg, 0.074 mmol) were dissolved in 1,4-dioxane (400 μL) under N₂. The mixture was heated at 105° C. for 90 min., then cooled to r.t., filtered through a plug of diatomaceous earth and concentrated. The crude product was dissolved in 1:1 DCM/TFA (1 mL) and the mixture was stirred at r.t. for 30 min., then diluted with MeCN and purified using prep.-LCMS (XBRIDGE® C₁₈ column, eluting with a gradient of MeCN/water containing 0.1% TFA, at flow rate of 60 mL/min.) to afford the title compound.

### Example 37. LCMS calc. for C₂₇H₂₂ClF₂N₆O₂S (M+H)⁺ m/z=567.1; found 567.1

The compound is understood to comprise a mixture of atropisomers, i.e., N-(8-(Rₐ)-(2-amino-3-cyano-7-fluorobenzo[b]thiophen-4-yl)-7-chloro-9-fluoro-4-((R)-1-methyl-2-oxopyrrolidin-3-yl)-5,6-dihydro-4H-benzo[de][1,6]naphthyridin-2-yl)acetamide; and N-(8-(Sₐ)-(2-amino-3-cyano-7-fluorobenzo[b]thiophen-4-yl)-7-chloro-9-fluoro-4-((R)-1-methyl-2-oxopyrrolidin-3-yl)-5,6-dihydro-4H-benzo[de][1,6]naphthyridin-2-yl)acetamide.

The alternative isomers, i.e., N-(8-(Rₐ)-(2-amino-3-cyano-7-fluorobenzo[b]thiophen-4-yl)-7-chloro-9-fluoro-4-((S)-1-methyl-2-oxopyrrolidin-3-yl)-5,6-dihydro-4H-benzo[de][1,6]naphthyridin-2-yl)acetamide; and N-(8-(Sₐ)-(2-amino-3-cyano-7-fluorobenzo[b]thiophen-4-yl)-7-chloro-9-fluoro-4-((S)-1-methyl-2-oxopyrrolidin-3-yl)-5,6-dihydro-4H-benzo[de][1,6]naphthyridin-2-yl)acetamide; are prepared by an analogous procedure using the (S)-enantiomer of Intermediate 10 as starting material.

### Example 38a and 38b. 2-Amino-4-(7-chloro-2-((difluoromethoxy)methyl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

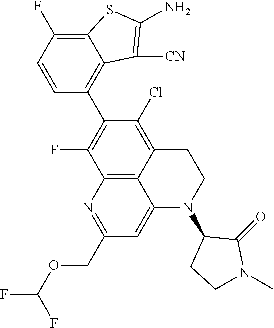 [SMILES: CN1CC[C@@H](N2CCc3c(Cl)c(-c4ccc(F)c5sc(N)c(C#N)c45)c(F)c4nc(COC(F)F)cc2c34)C1=O]

### Step 1. (R)-3-(8-Bromo-7-chloro-9-fluoro-2-vinyl-5,6-dihydro-4H-benzo[de][1,6]naphthyridin-4-yl)-1-methylpyrrolidin-2-one

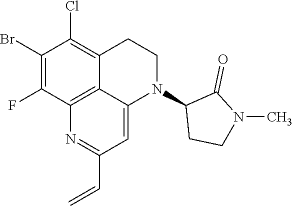 [SMILES: C=Cc1cc2c3c(c(Cl)c(Br)c(F)c3n1)CCN2[C@@H]1CCN(C)C1=O]

In a 2 dram vial, Intermediate 10 (600 mg, 1.14 mmol), 4,4,5,5-tetramethyl-2-vinyl-1,3,2-dioxaborolane (291 μL, 1.72 mmol), Pd(Ph₃P)₄ (132 mg, 0.114 mmol), and Cs₂CO₃ (1120 mg, 3.43 mmol) were dissolved in 1,4-dioxane (4.8 mL)/water (0.95 mL) under N₂. The mixture was heated at 95° C. for 20 min. The mixture was diluted with water and extracted with DCM. The organic layer was dried over MgSO₄ and concentrated. The crude product was purified by FCC (0-100% EtOAc in heptane) to provide the title compound (310 mg, 64%). LCMS calc. for C₁₃H₁₇BrClFN₃O (M+H)⁺ m/z=424.0, 426.0; found 424.0, 426.0.

### Step 2. (R)-8-Bromo-7-chloro-9-fluoro-4-(1-methyl-2-oxopyrrolidin-3-yl)-5,6-dihydro-4H-benzo[de][1,6]naphthyridine-2-carbaldehyde

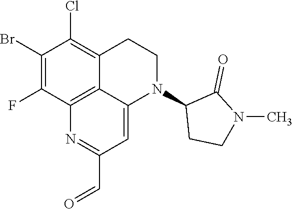 [SMILES: CN1CC[C@@H](N2CCc3c(Cl)c(Br)c(F)c4nc(C=O)cc2c34)C1=O]

In a 2 dram vial, (R)-3-(8-bromo-7-chloro-9-fluoro-2-vinyl-5,6-dihydro-4H-benzo[de][1,6]naphthyridin-4-yl)-1-methylpyrrolidin-2-one (228 mg, 0.538 mmol) was dissolved in THF (4.8 mL) open to air. Osmium tetroxide (4% in water, 422 μL, 0.054 mmol) was added and the mixture was stirred at r.t. for 3 min. NaIO₄ (288 mg, 1.34 mmol) was added to the mixture at once as a powder. The mixture was capped under N₂ and stirred for 2 h at r.t. The mixture was diluted with water and extracted with DCM. The organic layer was dried over MgSO₄ and concentrated. The crude product was purified by FCC (0-100% EtOAc in heptanes, followed by 0-100% heptane acetone) to provide the title compound (192 mg, 84%) as a light-yellow solid. LCMS calc. for C₁₇H₁₅BrClFN₃O₂(M+H)⁺ m/z=426.0, 428.0; found 426.0, 428.0.

### Step 3. (R)-3-(8-Bromo-7-chloro-9-fluoro-2-(hydroxymethyl)-5,6-dihydro-4H-benzo[de][1,6]naphthyridin-4-yl)-1-methylpyrrolidin-2-one

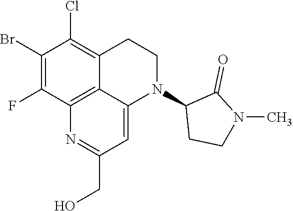 [SMILES: CN1CC[C@@H](N2CCc3c(Cl)c(Br)c(F)c4nc(CO)cc2c34)C1=O]

In a 40 mL vial, (R)-8-bromo-7-chloro-9-fluoro-4-(1-methyl-2-oxopyrrolidin-3-yl)-5,6-dihydro-4H-benzo[de][1,6]naphthyridine-2-carbaldehyde (192 mg, 0.450 mmol) was dissolved in THF (4.5 mL)/MeOH (2.2 mL) open to air and cooled to 0° C. NaBH₄ (18 mg, 0.476 mmol) was added to the mixture in one portion. The mixture was stirred at 0° C. for 30 min., then carefully quenched at with sat. aq. NaHCO₃. The mixture was extracted with EtOAc. The organic layer was washed with water and brine, dried over MgSO₄ and concentrated. The crude product was used in the next step without further purification (170 mg, 88%). LCMS calc. for C₁₇H₁₇BrClFN₃O₂(M+H)⁺ m/z=428.0, 430.0; found 428.0, 430.0.

### Step 4. (R)-3-(8-Bromo-7-chloro-2-((difluoromethoxy)methyl)-9-fluoro-5,6-dihydro-4H-benzo[de][1,6]naphthyridin-4-yl)-1-methylpyrrolidin-2-one

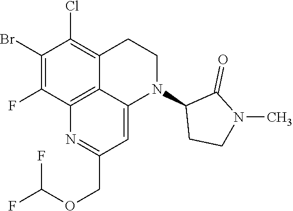 [SMILES: CN1CC[C@@H](N2CCc3c(Cl)c(Br)c(F)c4nc(COC(F)F)cc2c34)C1=O]

To a solution of (R)-3-(8-bromo-7-chloro-9-fluoro-2-(hydroxymethyl)-5,6-dihydro-4H-benzo[de][1,6]naphthyridin-4-yl)-1-methylpyrrolidin-2-one (11 mg, 0.025 mmol) in DCM (250 μL) were added NaOH (20% in water, 26 μL, 0.153 mmol) and trimethyl(bromodifluoromethyl)silane (12 μL, 0.076 mmol) and the mixture was stirred at r.t. for 2 h. The mixture was then concentrated and purified by prep.-SFC (5-25% CO₂ in MeOH, 2-Pic column, 140 mL/min. flow rate) to provide the title compound. LCMS calc. for C₁₃H₁₇BrClF₃N₃O₂ (M+H)⁺ m/z=478.0, 480.0; found 478.0, 480.0.

### Step 5. 2-Amino-4-(7-chloro-2-((difluoromethoxy)methyl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile

In a 1 dram vial, (R)-3-(8-bromo-7-chloro-2-((difluoromethoxy)methyl)-9-fluoro-5,6-dihydro-4H-benzo[de][1,6]naphthyridin-4-yl)-1-methylpyrrolidin-2-one (6.0 mg, 0.013 mmol), tert-butyl (3-cyano-4-(5,5-dimethyl-1,3,2-dioxaborinan-2-yl)-7-fluorobenzo[b]thiophen-2-yl)carbamate (10 mg, 0.025 mmol), dichloro[bis(diphenylphosphinophenyl)ether]palladium(II) (2.2 mg, 3.1 μmol), and K₂CO₃ (4 mg, 0.031 mmol) were dissolved in 1,4-dioxane (100 μL) under N₂. The mixture was stirred at 100° C. for 1 h. The mixture was diluted with MeCN and filtered. The filtrate was diluted with DCM and the layers were separated. The organic layer was washed with brine and concentrated. To the crude was added TFA (1 mL), and the resulting mixture was stirred at r.t. for 30 min., then concentrated. The residue was dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 38a. Diastereomer 1. Peak 1. LCMS calc. for C₂₇H₂₁ClF₄N₅O₂S (M+H)⁺ m/z=590.1; found 590.1. This is the potent peak.

Example 38b. Diastereomer 2. Peak 2. LCMS calc. for C₂₇H₂₁ClF₄N₅O₂S (M+H)⁺ m/z=590.1; found 590.1.

The compounds are understood to comprise atropisomers, i.e., (Rₐ)-2-amino-4-(7-chloro-2-((difluoromethoxy)methyl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((difluoromethoxy)methyl)-9-fluoro-4-((R)-1-methyl-2-oxopyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((difluoromethoxy)methyl)-9-fluoro-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((difluoromethoxy)methyl)-9-fluoro-4-((S)-1-methyl-2-oxopyrrolidin-3-yl)-5,6-dihydro-4H-benzo[de][1,6]naphthyridin-8-yl)-7-fluorobenzo[b]thiophene-3-carbonitrile; are prepared by an analogous procedure using the (S)-enantiomer of Intermediate 10 as starting material.

### Example 39a and 39b. 2-Amino-4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile

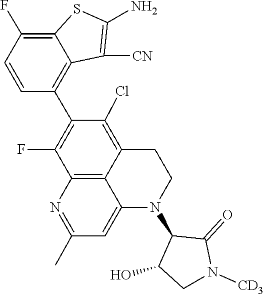 [SMILES: [2H]C([2H])([2H])N1C[C@H](O)[C@@H](N2CCc3c(Cl)c(-c4ccc(F)c5sc(N)c(C#N)c45)c(F)c4nc(C)cc2c34)C1=O]

### Step 1. Methyl (2R)-2-(((Benzyloxy)carbonyl)amino)-2-(oxiran-2-yl)acetate

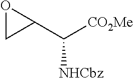 [SMILES: COC(=O)[C@H](NC(=O)OCc1ccccc1)C1CO1]

To a solution of methyl (R)-2-(((benzyloxy)carbonyl)amino)but-3-enoate (1.0 g, 4.0 mmol) in DCM (16 mL) was added m-CPBA (1.98 g, 8.02 mmol) and the mixture was heated at 40° C. for 5 h. The reaction was quenched with sat. aq. Na₂S₂O₃ and extracted with DCM. The organic layer was washed with sat. aq. NaHCO₃, dried over Na₂SO₄ and concentrated. The crude product was used in the next step without further purification. The product is a ˜5:1 mixture of diastereomers. LCMS calc. for C₁₃H₁₆NO₅ (M+H)⁺ m/z=266.1; found 266.1.

### Step 2. Methyl (2R)-4-azido-2-(((benzyloxy)carbonyl)amino)-3-hydroxybutanoate

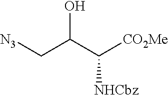 [SMILES: COC(=O)[C@H](NC(=O)OCc1ccccc1)C(O)CN=[N+]=[N-]]

To a solution of methyl (2R)-2-(((benzyloxy)carbonyl)amino)-2-(oxiran-2-yl)acetate (1.3 g, 4.9 mmol) in DMF (12 mL) were added ammonium chloride (0.26 g, 4.90 mmol) and sodium azide (0.96 g, 14.7 mmol) and the mixture was heated at 60° C. for 2 h, then quenched with water. The mixture was extracted with EtOAc and the organic layer was washed with water and brine, dried over Na₂SO₄ and concentrated. The crude product was used in the next step without further purification. LCMS calc. for C₁₃H₁₇N₄O₅ (M+H)⁺ m/z=309.1; found 309.1.

### Step 3. Benzyl ((3R,4S)-4-hydroxy-2-oxopyrrolidin-3-yl)carbamate

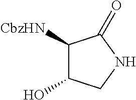 [SMILES: O=C(N[C@H]1C(=O)NC[C@@H]1O)OCc1ccccc1]

To a solution of methyl (2R)-4-azido-2-(((benzyloxy)carbonyl)amino)-3-hydroxybutanoate (1.5 g, 4.87 mmol) in THF (16 mL), MeOH (21.6 mL) and water (10.8 mL) was added triphenylphosphine (1.28 g, 4.87 mmol) and the mixture was heated at 90° C. for 3 h. The mixture was concentrated and the crude product was purified by prep.-LCMS (XBRIDGE® C₁₈ column, eluting with a gradient of MeCN/water containing 0.1% TFA, at flow rate of 60 mL/min.) to afford the title compound as a single isomer. LCMS calc. for C₁₂H₁₅N₂O₄ (M+H)⁺ m/z=251.1; found 251.1.

### Step 4. Benzyl ((3R,4S)-4-((tert-butyldiphenylsilyl)oxy)-2-oxopyrrolidin-3-yl)carbamate

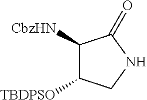

To a solution of benzyl ((3R,4S)-4-hydroxy-2-oxopyrrolidin-3-yl)carbamate (300 mg, 1.2 mmol) in 1-methylimidazole (1.1 mL, 14.4 mmol) was added tert-butyldiphenylchlorosilane (320 μL, 1.26 mmol) and the mixture was stirred at r.t. for 30 min., then partitioned between EtOAc and brine. The layers were separated and the organic layer was washed with 1N HCl, dried over Na₂SO₄ and concentrated. The crude product was used in the next step without further purification. LCMS calc. for C₂₃H₃₃N₂O₄Si (M+H)⁺ m/z=489.2; found 489.2.

### Step 5. (3R,4S)-3-Amino-4-((tert-butyldiphenylsilyl)oxy)pyrrolidin-2-one

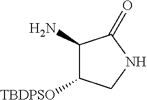

To a solution of benzyl ((3R,4S)-4-((tert-butyldiphenylsilyl)oxy)-2-oxopyrrolidin-3-yl)carbamate (586 mg, 1.2 mmol) in MeOH (12 mL) was added palladium hydroxide (20% w/w, 84 mg, 0.12 mmol) and the reaction flask was evacuated and backfilled with hydrogen gas from a balloon. The mixture was stirred at r.t. overnight, then filtered through diatomaceous earth. The filtrate was concentrated and the crude product was used in the next step without further purification. LCMS calc. for C₂₀H₂₇N₂O₂Si (M+H)⁺ m/z=355.2; found 355.2.

### Step 6. (3R,4S)-4-((tert-Butyldiphenylsilyl)oxy)-3-((diphenylmethylene)amino)pyrrolidin-2-one

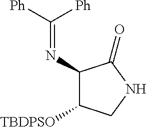

To a solution of (3R,4S)-3-amino-4-((tert-butyldiphenylsilyl)oxy)pyrrolidin-2-one (360 mg, 1.01 mmol) in DCM (4 mL) was added benzophenone imine (180 μL, 1.07 mmol) and the mixture was stirred at r.t. for 2 h. The mixture was filtered and concentrated, then purified by FCC (0-100% EtOAc in hexanes) to provide the title compound (200 mg, 38%). LCMS calc. for C₃₃H₃₅N₂O₂Si (M+H)⁺ m/z=519.2; found 519.2.

### Step 7. (3R,4S)-3-Amino-4-((tert-butyldiphenylsilyl)oxy)-1-(methyl-d₃)pyrrolidin-2-one

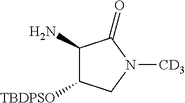

To a solution of (3R,4S)-4-((tert-butyldiphenylsilyl)oxy)-3-((diphenylmethylene)amino)pyrrolidin-2-one (194 mg, 0.37 mmol) in THF (1.9 mL) was added NaH (60% w/w in mineral oil, 45 mg, 1.12 mmol) at 0° C. and the mixture was stirred at this temperature for 30 min., then iodomethane-d₃ (116 μL, 1.87 mmol) was added and the mixture was warmed to r.t. and stirred for 1 h. HCl (1 N, 1.8 mL) was added and the mixture was stirred an additional 1 h at r.t. The mixture was then extracted with EtOAc. The aqueous layer was diluted with 1 N NaOH until the pH was ˜7, then the mixture was extracted with EtOAc. The combined organic layers were dried over Na₂SO₄ and concentrated. The crude product was purified by FCC (0-100% MeOH in DCM) to provide the title compound. LCMS calc. for C₂₁H₂₆D₃N₂O₂Si (M+H)⁺ m/z=372.2; found 372.2.

### Step 8. (3R,4S)-3-(8-Bromo-7-chloro-9-fluoro-2-methyl-5,6-dihydro-4H-benzo[de][1,6]naphthyridin-4-yl)-4-((tert-butyldiphenylsilyl)oxy)-1-(methyl-d₃)pyrrolidin-2-one

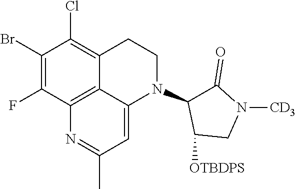

To a solution of Intermediate 4 (40 mg, 0.11 mmol) in DCM (570 μL) were added (3R,4S)-3-amino-4-((tert-butyldiphenylsilyl)oxy)-1-(methyl-d₃)pyrrolidin-2-one (51 mg, 0.14 mmol), PPTS (29 mg, 0.11 mmol) and NaBH(OAc)₃ (97 mg, 0.46 mmol) and the mixture was stirred at r.t. for 2 h. The reaction was quenched with sat. aq. NaHCO₃ and extracted with DCM. The organic layer was dried over Na₂SO₄ and concentrated. The crude product was purified by FCC (0-40% MeOH in DCM) to provide the title compound (68 mg, 89%). LCMS calc. for C₃₃H₃₂D₃BrClFN₃O₂Si (M+H)⁺ m/z=669.1, 671.1; found 669.1, 671.1.

### Step 9. tert-Butyl (4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-3-cyano-7-fluorobenzo[b]thiophen-2-yl)carbamate

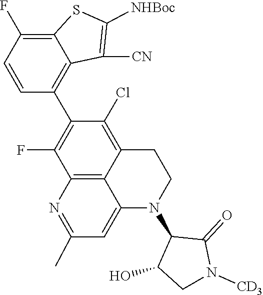 [SMILES: [2H]C([2H])([2H])N1C[C@H](O)[C@@H](N2CCc3c(Cl)c(-c4ccc(F)c5sc(NC(=O)OC(C)(C)C)c(C#N)c45)c(F)c4nc(C)cc2c34)C1=O]

To a mixture of tert-butyl (3-cyano-4-(5,5-dimethyl-1,3,2-dioxaborinan-2-yl)-7-fluorobenzo[b]thiophen-2-yl)carbamate (103 mg, 0.25 mmol), (3R,4S)-3-(8-bromo-7-chloro-9-fluoro-2-methyl-5,6-dihydro-4H-benzo[de][1,6]naphthyridin-4-yl)-4-((tert-butyldiphenylsilyl)oxy)-1-(methyl-d₃)pyrrolidin-2-one (68 mg, 0.10 mmol), K₂CO₃ (42 mg, 0.30 mmol) and dichloro[bis(diphenylphosphinophenyl)ether]palladium(II) (22 mg, 0.03 mmol) was added 1,4-dioxane (670 μL) and the reaction flask was evacuated, back filled with N₂, then stirred at 100° C. for 1 h. The reaction was quenched with water and extracted with EtOAc. The organic layer was washed with brine, dried over Na₂SO₄ and concentrated. The crude product was dissolved in THF (1 mL) and TBAF (1.0 M in THF, 510 μL, 0.51 mmol) was added. The mixture was stirred at r.t. for 30 min., then concentrated. The crude product was purified by FCC (10% MeOH in DCM) to provide the title compound. LCMS calc. for C₃₁H₂₆D₃ClF₂N₅O₄S (M+H)⁺ m/z=643.2; found 643.2.

### Step 10. 2-Amino-4-(7-chloro-9-fluoro-4-((3R,4S)-4-hydroxy-1-methyl-2-oxopyrrolidin-3-yl)-2-methyl-5,6-dihydro-4H-benzo[de][1,6]naphthyridin-8-yl)-7-fluorobenzo[b]thiophene-3-carbonitrile

A solution of tert-butyl (4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-3-cyano-7-fluorobenzo[b]thiophen-2-yl)carbamate (89 mg, 0.10 mmol) was dissolved in DCM (0.5 mL) and TFA (0.5 mL) was added. After stirring at r.t. for 30 min., then mixture was concentrated, then diluted with MeCN and purified by prep. HPLC (XBRIDGE® C₁₈ column, eluting with a gradient of MeOH/water containing 0.1% TFA, at flow rate of 60 mL/min.) to afford the title compound as two peaks.

Example 39a. Diastereomer 1. Peak 1. LCMS calc. for C₂₆H₁₈D₃ClF₂N₅O₂S (M+H)⁺ m/z=540.1; found 540.1. This is the potent peak.

Example 39b. Diastereomer 2. Peak 2. LCMS calc. for C₂₆H₁₈D₃ClF₂N₅O₂S (M+H)⁺ m/z=540.1; found 540.1.

The compounds are understood to comprise atropisomers, i.e., (Rₐ)-2-amino-4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,4R)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,4R)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; are prepared by an analogous procedure using methyl (S)-2-(((benzyloxy)carbonyl)amino)but-3-enoate as starting material.

Further isomers are prepared by modification of the foregoing procedures using appropriate starting materials, i.e., (Rₐ)-2-amino-4-(7-chloro-9-fluoro-4-((3R,4R)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,4R)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,4S)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,4S)-4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile.

### Example 40. 2-Amino-4-(7-chloro-4-((3R,4S)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile

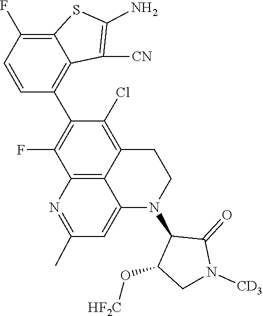

To a solution of tert-butyl (4-(7-chloro-9-fluoro-4-((3R,4S)-4-hydroxy-1-(methyl-d₃)-2-oxopyrrolidin-3-yl)-2-methyl-5,6-dihydro-4H-benzo[de][1,6]naphthyridin-8-yl)-3-cyano-7-fluorobenzo[b]thiophen-2-yl)carbamate (Example 39, Step 9, 29 mg, 0.045 mmol) in MeCN (250 μL) was added CuI (1.7 mg, 9 μmol) and the mixture was heated at 50° C. 2-(fluorosulfonyl)difluoroacetic acid (7 μL, 0.07 mmol) was added and the mixture was stirred at this temperature for 30 min. The mixture was then concentrated and redissolved in 1:1 DCM/TFA (1 mL). After stirring at r.t. for 30 min., the mixture was concentrated, diluted with MeOH and purified by prep. HPLC (XBRIDGE® C₁₈ column, eluting with a gradient of MeOH/water containing 0.1% TFA, at flow rate of 60 mL/min.) to afford the title compound. LCMS calc. for C₂₇H₁₈D₃ClF₄N₅O₂S (M+H)⁺ m/z=593.1; found 593.1.

The compound is understood to comprise a mixture of atropisomers, i.e., (Rₐ)-2-amino-4-(7-chloro-4-((3R,4S)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3R,4S)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-4-((3S,4R)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,4R)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; are prepared by an analogous procedure using tert-butyl (4-(7-chloro-9-fluoro-4-((3S,4R)-4-hydroxy-1-(methyl-d₃)-2-oxopyrrolidin-3-yl)-2-methyl-5,6-dihydro-4H-benzo[de][1,6]naphthyridin-8-yl)-3-cyano-7-fluorobenzo[b]thiophen-2-yl)carbamate as starting material.

Further isomers are prepared by appropriate modification of the above procedures, e.g., employing methods for the stereoselective synthesis or inversion of alcohols, i.e., (Rₐ)-2-amino-4-(7-chloro-4-((3S,4S)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,4S)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3R,4R)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3R,4R)-4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Example 41a and 41b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

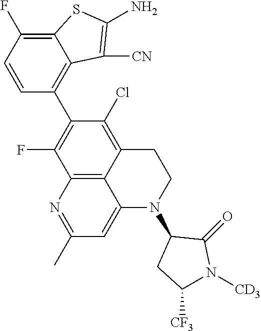 [SMILES: [2H]C([2H])([2H])N1C(=O)[C@H](N2CCc3c(Cl)c(-c4ccc(F)c5sc(N)c(C#N)c45)c(F)c4nc(C)cc2c34)C[C@H]1C(F)(F)F]

### Step 1. tert-Butyl (4-(7-chloro-9-fluoro-2-methyl-4-((3R,5S)-1-(methyl-d3)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate

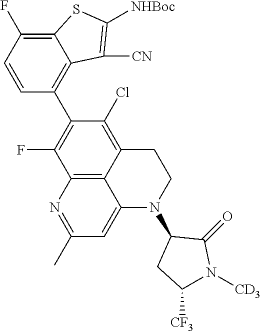 [SMILES: [2H]C([2H])([2H])N1C(=O)[C@H](N2CCc3c(Cl)c(-c4ccc(F)c5sc(NC(=O)OC(C)(C)C)c(C#N)c45)c(F)c4nc(C)cc2c34)C[C@H]1C(F)(F)F]

To a solution of Intermediate 9 (395 mg, 0.69 mmol) and Intermediate 11 (319 mg, 1.72 mmol) in DCM/THF/HFIP (3:3:1, 7 mL total volume) were added NaBH(OAc)₃ (584 mg, 2.76 mmol) and AcOH (425 μL, 5.51 mmol). The mixture was stirred at r.t. for 1 h. The reaction was quenched with sat. aq. NaHCO₃ and extracted with EtOAc. The organic layer was washed with brine, dried over Na₂SO₄ and concentrated. The crude product was purified by FCC (0-10% MeOH in DCM) to provide the title compound (367 mg, 73%). LCMS calc. for C₃₂H₂₅D₃ClF₅N₅O₃S (M+H)⁺ m/z=695.2; found 695.2.

### Step 2. 2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To a solution of tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (302 mg, 0.43 mmol) in DCM (3 mL) was added TFA (3 mL) and the mixture was stirred at r.t. for 30 min., then concentrated. The residue was partitioned between EtOAc and sat. aq. NaHCO₃ and the layers were separated. The aqueous phase was pH adjusted to >7 with solid Na₂CO₃, then the mixture was extracted with DCM. The combined organic phases were dried over Na₂SO₄ and concentrated. The crude product was purified by FCC (0-100% EtOAc in hexanes) then further purified by prep. HPLC (XBRIDGE® C₁₈ column, eluting with a gradient of MeCN/water containing 0.1% NH₄OH, at flow rate of 60 mL/min.) to afford the title compound as two peaks.

Example 41a. Diastereomer 1. Peak 1. LCMS calc. for C₂₇H₁₇D₃ClF₅N₅OS (M+H)⁺ m/z=595.1; found 595.1. This is the potent peak. ¹H NMR (500 MHz, DMSO) b 8.02 (s, 2H), 7.24 (dd, J=8.4, 5.2 Hz, 1H), 7.14 (t, J=8.9 Hz, 1H), 6.86 (s, 1H), 5.08 (t, J=9.3 Hz, 1H), 4.61 (p, J=8.1 Hz, 1H), 3.41 (ddd, J=12.5, 8.2, 4.8 Hz, 1H), 3.29 (s, 1H), 3.25 (dt, J=11.9, 5.7 Hz, 1H), 3.15 (tt, J=14.8, 6.7 Hz, 2H), 2.65 (dd, J=14.2, 9.1 Hz, 1H), 2.54 (s, 3H).

Example 41b. Diastereomer 2. Peak 2. LCMS calc. for C₂₇H₁₇D₃ClF₅N₅OS (M+H)⁺ m/z=595.1; found 595.1

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the (3S,5R)-diastereomer of Intermediate 11 as starting material.

Further isomers are prepared by modification of the foregoing procedures using appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Examples 42-46

Compounds in Table 6 were prepared according to the general procedure described for Example 41, using the appropriate lactam starting material.

TABLE 6

| # | Structure | Name | [M + H] |
|---|---|---|---|
| 42a/b | 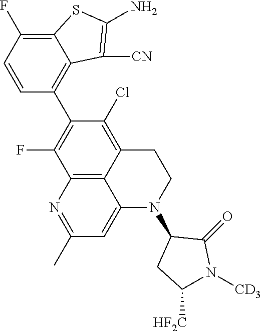 | 2-amino-4-(7-chloro-4-((3R,5S)-5- (difluoromethyl)-1-(methyl-d₃)-2- oxopyrrolidin-3-yl)-9-fluoro-2-methyl- 5,6-dihydro-4H- benzo[de][1,6]naphthyridin-8-yl)-7- fluorobenzo[b]thiophene-3-carbonitrile | 577.1 |
| 43a/b | 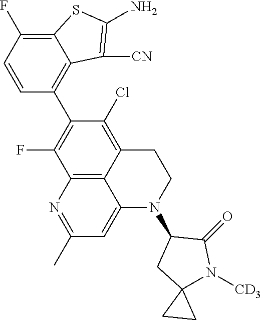 [SMILES: [2H]C([2H])([2H])N1C(=O)[C@H](N2CCc3c(Cl)c(-c4ccc(F)c5sc(N)c(C#N)c45)c(F)c4nc(C)cc2c34)CC12CC2] | 2-amino-4-(7-chloro-9-fluoro-2-methyl- 4-((R)-4-(methyl-d₃)-5-oxo-4- azaspiro[2.4]heptan-6-yl)-5,6-dihydro- 4H-benzo[de][1,6]naphthyridin-8-yl)-7- fluorobenzo[b]thiophene-3-carbonitrile | 553.1 |
| 44a/b/c/d | 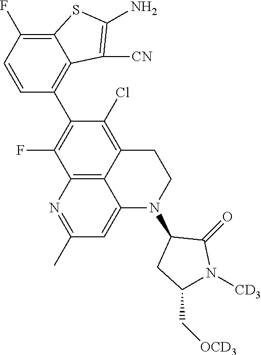 | 2-amino-4-(7-chloro-9-fluoro-4- ((3R,5S)-5-((methoxy-d₃)methyl)-1- (methyl-d₃)-2-oxopyrrolidin-3-yl)-2- methyl-5,6-dihydro-4H- benzo[de][1,6]naphthyridin-8-yl)-7- fluorobenzo[b]thiophene-3-carbonitrile | 574.1 |
| 45a/b | 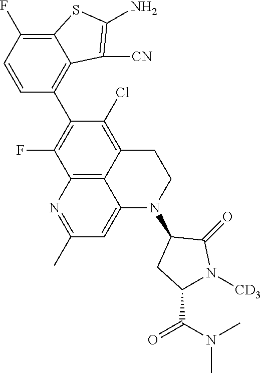 [SMILES: [2H]C([2H])([2H])N1C(=O)[C@H](N2CCc3c(Cl)c(-c4ccc(F)c5sc(N)c(C#N)c45)c(F)c4nc(C)cc2c34)C[C@H]1C(=O)N(C)C] | (2S,4R)-4-(8-(2-amino-3-cyano-7- fluorobenzo[b]thiophen-4-yl)-7-chloro-9- fluoro-2-methyl-5,6-dihydro-4H- benzo[de][1,6]naphthyridin-4-yl)-N,N- dimethyl-1-(methyl-d₃)-5-oxopyrrolidine- 2-carboxamide | 598.2 |
| 46a/b | 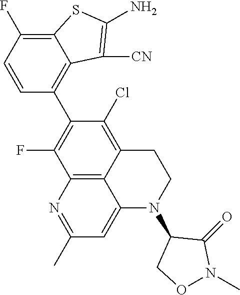 [SMILES: Cc1cc2c3c(c(Cl)c(-c4ccc(F)c5sc(N)c(C#N)c45)c(F)c3n1)CCN2[C@@H]1CON(C)C1=O] | 2-amino-4-(7-chloro-9-fluoro-2-methyl- 4-((R)-2-methyl-3-oxoisoxazolidin-4-yl)- 5,6-dihydro-4H- benzo[de][1,6]naphthyridin-8-yl)-7- fluorobenzo[b]thiophene-3-carbonitrile | 526.1 |

The compounds of Examples 42, 43, 45 and 46 of Table 6 are understood to comprise atropisomers, i.e.,

### Examples 42a and 42b

(Rₐ)-2-amino-4-(7-chloro-4-((3R,5S)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3R,5S)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-4-((3S,5R)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,5R)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; are prepared by an analogous procedure using the (3S,5R)-diastereomer of Intermediate 11 as starting material.

Further isomers are prepared by using modifications to the procedure and employing appropriate starting materials, i.e., (Rₐ)-2-amino-4-(7-chloro-4-((3R,5R)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3R,5R)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3S,5S)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,5S)-5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Examples 43a and 43b

(Rₐ)-2-amino-4-(7-chloro-9-fluoro-2-methyl-4-((R)-4-(methyl-d₃)-5-oxo-4-azaspiro[2.4]heptan-6-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((R)-4-(methyl-d₃)-5-oxo-4-azaspiro[2.4]heptan-6-yl)-5,6-dihydro-4H-benzo[de][1,6]naphthyridin-8-yl)-7-fluorobenzo[b]thiophene-3-carbonitrile.

The alternative isomers, i.e.,
(Rₐ)-2-amino-4-(7-chloro-9-fluoro-2-methyl-4-((S)-4-(methyl-d₃)-5-oxo-4-azaspiro[2.4]heptan-6-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((S)-4-(methyl-d₃)-5-oxo-4-azaspiro[2.4]heptan-6-yl)-5,6-dihydro-4H-benzo[de][1,6]naphthyridin-8-yl)-7-fluorobenzo[b]thiophene-3-carbonitrile; are prepared by an analogous procedure using the alternative enantiomer as starting material.

### Examples 45a and 45b

(2S,4R)-4-(8-(Rₐ)-(2-amino-3-cyano-7-fluorobenzo[b]thiophen-4-yl)-7-chloro-9-fluoro-2-methyl-5,6-dihydro-4H-benzo[de][1,6]naphthyridin-4-yl)-N,N-dimethyl-1-(methyl-d₃)-5-oxopyrrolidine-2-carboxamide; and (2S,4R)-4-(8-(Sₐ)-(2-amino-3-cyano-7-fluorobenzo[b]thiophen-4-yl)-7-chloro-9-fluoro-2-methyl-5,6-dihydro-4H-benzo[de][1,6]naphthyridin-4-yl)-N,N-dimethyl-1-(methyl-d₃)-5-oxopyrrolidine-2-carboxamide.

The alternative isomers, i.e., (2R,4S)-4-(8-(Rₐ)-(2-amino-3-cyano-7-fluorobenzo[b]thiophen-4-yl)-7-chloro-9-fluoro-2-methyl-5,6-dihydro-4H-benzo[de][1,6]naphthyridin-4-yl)-N,N-dimethyl-1-(methyl-d₃)-5-oxopyrrolidine-2-carboxamide; and (2R,4S)-4-(8-(S)-(2-amino-3-cyano-7-fluorobenzo[b]thiophen-4-yl)-7-chloro-9-fluoro-2-methyl-5,6-dihydro-4H-benzo[de][1,6]naphthyridin-4-yl)-N,N-dimethyl-1-(methyl-d₃)-5-oxopyrrolidine-2-carboxamide; are prepared by an analogous procedure using the alternative diastereomer as starting material.

### Examples 46a and 46b

(Rₐ)-2-amino-4-(7-chloro-9-fluoro-2-methyl-4-((R)-2-methyl-3-oxoisoxazolidin-4-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((R)-2-methyl-3-oxoisoxazolidin-4-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-methyl-4-((S)-2-methyl-3-oxoisoxazolidin-4-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((S)-2-methyl-3-oxoisoxazolidin-4-yl)-5,6-dihydro-4H-benzo[de][1,6]naphthyridin-8-yl)-7-fluorobenzo[b]thiophene-3-carbonitrile; are prepared by an analogous procedure using the alternative enantiomer as starting material.

The compounds of Example 44 of Table 6 are understood to comprise atropisomers and/or diastereoisomers, i.e.,

### Examples 44a, 44b, 44c and 44d

(Rₐ)-2-amino-4-(7-chloro-9-fluoro-4-((3R,5S)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile; and/or (Sₐ)-2-amino-4-(7-chloro-9-fluoro-4-((3S,5R)-5-((methoxy-d₃)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile.

Individual diastereoisomers are prepared by using appropriate diastereoisomers as starting material and/or using appropriate chromatographic or other separation methods.

### Example 47a and 47b. 2-Amino-4-(7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

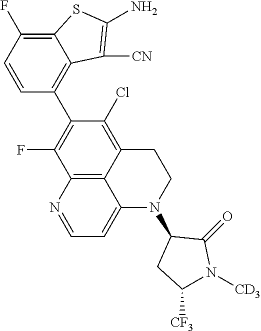 [SMILES: [2H]C([2H])([2H])N1C(=O)[C@H](N2CCc3c(Cl)c(-c4ccc(F)c5sc(N)c(C#N)c45)c(F)c4nccc2c34)C[C@H]1C(F)(F)F]

### Step 1. (3R,5S)-3-(8-Bromo-7-chloro-9-fluoro-5,6-dihydro-4H-benzo[de][1,6]naphthyridin-4-yl)-1-(methyl-d₃)-5-(trifluoromethyl)pyrrolidin-2-one

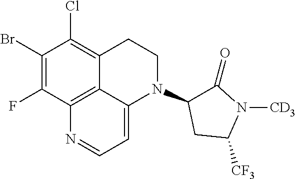 [SMILES: [2H]C([2H])([2H])N1C(=O)[C@H](N2CCc3c(Cl)c(Br)c(F)c4nccc2c34)C[C@H]1C(F)(F)F]

In a 2 dram vial, Intermediate 13 (42.3 mg, 0.071 mmol), (dppf)PdCl₂ (8 mg, 11 μmol), NaBH₄ (9 mg, 0.24 mmol), and TMEDA (36 μL, 0.24 mmol) were dissolved in THF (710 μL) under N₂. The mixture was heated at 45° C. for 2 h, then concentrated. The crude product was purified by prep. SFC (2-PIC column, 5-25% CO₂ in MeOH, 140 mL/min. flow rate) to provide the title compound. LCMS calc. for C₁₇H₁₁D₃BrClF₄N₃O (M+H)⁺ m/z=469.1, 471.1; found 469.1, 471.1.

### Step 2. 2-Amino-4-(7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-9-fluoro-5,6-dihydro-4H-benzo[de][1,6]naphthyridin-4-yl)-1-(methyl-d₃)-5-(trifluoromethyl)pyrrolidin-2-one (5 mg, 0.011 mmol), tert-butyl (3-cyano-4-(5,5-dimethyl-1,3,2-dioxaborinan-2-yl)-7-fluorobenzo[b]thiophen-2-yl)carbamate (9 mg, 0.023 mmol), dichloro[bis(diphenylphosphinophenyl)ether]palladium(II) (2 mg, 2.9 μmol), and K₂CO₃ (4 mg, 0.03 mmol) were dissolved in 1,4-dioxane (200 μL) under N₂. The mixture was then heated at 100° C. for 1 h, then diluted with MeCN and filtered. The filtrate was concentrated and the crude product dissolved in 1:1 DCM/TFA (1 mL). The mixture was stirred at r.t. for 30 min., then concentrated. The residue was diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 47a. Diastereomer 1. Peak 1. LCMS calc. for C₂₆H₁₅D₃ClF₅N₅OS (M+H)⁺ m/z=581.1; found 581.1. This is the potent peak.

Example 47b. Diastereomer 2. Peak 2. LCMS calc. for C₂₆H₁₅D₃ClF₅N₅OS (M+H)⁺ m/z=581.1; found 581.1.

The compounds are understood to comprise atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 48a and 48b. 2-Amino-4-(7-chloro-2-(3-(dimethylamino)azetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

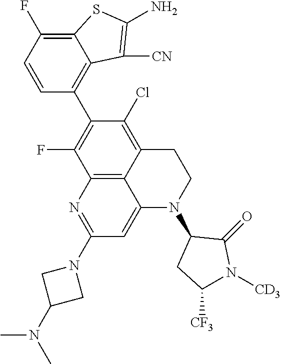 [SMILES: [2H]C([2H])([2H])N1C(=O)[C@H](N2CCc3c(Cl)c(-c4ccc(F)c5sc(N)c(C#N)c45)c(F)c4nc(N5CC(N(C)C)C5)cc2c34)C[C@H]1C(F)(F)F]

### Step 1. (3R,5S)-3-(8-Bromo-7-chloro-2-(3-(dimethylamino)azetidin-1-yl)-9-fluoro-5,6-dihydro-4H-benzo[de][1,6]naphthyridin-4-yl)-1-(methyl-d₃)-5-(trifluoromethyl)pyrrolidin-2-one

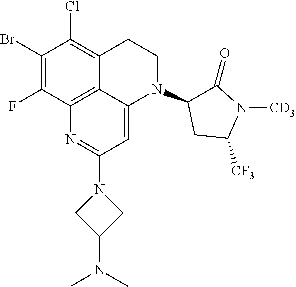 [SMILES: [2H]C([2H])([2H])N1C(=O)[C@H](N2CCc3c(Cl)c(Br)c(F)c4nc(N5CC(N(C)C)C5)cc2c34)C[C@H]1C(F)(F)F]

In a 1 dram vial, Intermediate 13 (38 mg, 0.064 mmol), N,N-dimethylazetidin-3-amine dihydrochloride (55 mg, 0.32 mmol), L-proline (12 mg, 0.11 mmol), CuI (12 mg, 0.064 mmol) and K₂CO₃ (88 mg, 0.64 mmol) were dissolved in DMSO (636 μL) under N₂. The mixture was heated at 85° C. for 15 min. The mixture was diluted with sat. aq. NH₄OH solution, then extracted with EtOAc. The organic layer was washed with 10% aq. LiCl, dried over MgSO₄ and concentrated. The crude product was used in the next step without further purification. LCMS calc. for C₂₂H₂₁D₃BrClF₄N₅O (M+H)⁺ m/z=567.1, 569.1; found 567.1, 569.1.

### Step 2. 2-Amino-4-(7-chloro-2-(3-(dimethylamino)azetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-2-(3-(dimethylamino)azetidin-1-yl)-9-fluoro-5,6-dihydro-4H-benzo[de][1,6]naphthyridin-4-yl)-1-(methyl-d₃)-5-(trifluoromethyl)pyrrolidin-2-one (20 mg, 0.035 mmol), tert-butyl (3-cyano-4-(5,5-dimethyl-1,3,2-dioxaborinan-2-yl)-7-fluorobenzo[b]thiophen-2-yl)carbamate (40 mg, 0.099 mmol), dichloro[bis(diphenylphosphinophenyl)ether]palladium(II) (10 mg, 0.014 mmol), and K₂CO₃ (39 mg, 0.282 mmol) were dissolved in 1,4-dioxane (400 μL) under N₂. The mixture was stirred at 100° C. for 1 h, then diluted with MeCN and filtered. The filtrate was concentrated and dissolved in 1:1 DCM/TFA (1 mL). After stirring at r.t. for 30 min., the mixture was concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 48a. Diastereomer 1. Peak 1. LCMS calc. for C₃₁H₂₅D₃ClF₅N₇OS (M+H)⁺ m/z=679.2; found 679.2. ¹H NMR (500 MHz, DMSO) b 8.03 (s, 2H), 7.21 (dd, J=8.4, 5.3 Hz, 1H), 7.14 (t, J=8.9 Hz, 1H), 5.94 (s, 1H), 5.09 (t, J=9.2 Hz, 1H), 4.63 (dd, J=11.2, 4.7 Hz, 1H), 4.38 (dd, J=17.4, 7.9 Hz, 2H), 4.31 (s, 1H), 4.24 (s, 2H), 3.44 (dt, J=12.5, 6.4 Hz, 1H), 3.25 (dt, J=12.9, 6.5 Hz, 1H), 3.08 (t, J=6.4 Hz, 2H), 2.83 (s, 6H), 2.64-2.51 (m, 2H). This is the potent peak.

Example 48b. Diastereomer 2. Peak 2. LCMS calc. for C₃₁H₂₅D₃ClF₅N₇OS (M+H)⁺ m/z=679.2; found 679.2.

The compounds are understood to comprise atropisomers, i.e., (Rₐ)-2-amino-4-(7-chloro-2-(3-(dimethylamino)azetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)azetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-(3-(dimethylamino)azetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)azetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-2-(3-(dimethylamino)azetidin-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(3-(dimethylamino)azetidin-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(3-(dimethylamino)azetidin-1-yl)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)azetidin-1-yl)-9-fluoro-4-((3S,5S)-1-(methyl-d₃)-2-oxo-5-(trifluoromethyl) pyrrolidin-3-yl)-5,6-dihydro-4H-benzo[de][1,6]naphthyridin-8-yl)-7-fluorobenzo[b]thiophene-3-carbonitrile;

### Examples 49-51

The compounds of Examples 49-51 in Table 7 were prepared according to the general procedure described for Example 48, using the appropriate alcohol starting material.

TABLE 7

| # | Structure | Name | [M + H] |
|---|---|---|---|
| 49a/b | 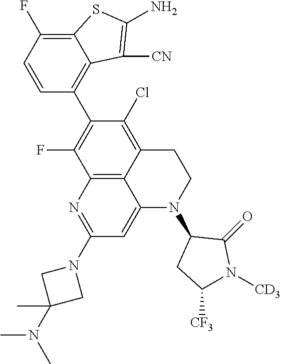 [SMILES: [2H]C([2H])([2H])N1C(=O)[C@H](N2CCc3c(Cl)c(-c4ccc(F)c5sc(N)c(C#N)c45)c(F)c4nc(N5CC(C)(N(C)C)C5)cc2c34)C[C@H]1C(F)(F)F] | 2-amino-4-(7-chloro-2-(3- (dimethylamino)-3-methylazetidin-1-yl)- 9-fluoro-4-((3R,5S)-1-(methyl-d₃)-2-oxo- 5-(trifluoromethyl)pyrrolidin-3-yl)-5,6- dihydro-4H-benzo[de][1,6]naphthyridin- 8-yl)-7-fluorobenzo[b]thiophene-3- carbonitrile | 693.2 |
| 50a/b | 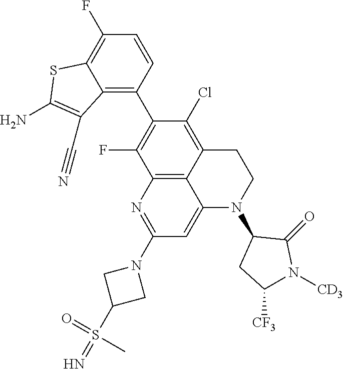 [SMILES: [2H]C([2H])([2H])N1C(=O)[C@H](N2CCc3c(Cl)c(-c4ccc(F)c5sc(N)c(C#N)c45)c(F)c4nc(N5CC(S(C)(=N)=O)C5)cc2c34)C[C@H]1C(F)(F)F] | 2-amino-4-(7-chloro-9-fluoro-4-((3R,5S)- 1-(methyl-d₃)-2-oxo-5- (trifluoromethyl)pyrrolidin-3-yl)-2-(3-(S- methylsulfonimidoyl)azetidin-1-yl)-5,6- dihydro-4H-benzo[de][1,6]naphthyridin- 8-yl)-7-fluorobenzo[b]thiophene-3- carbonitrile | 713.1 |
| 51 | 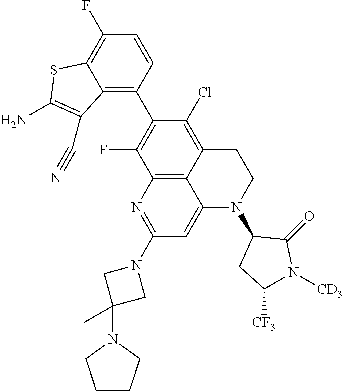 [SMILES: [2H]C([2H])([2H])N1C(=O)[C@H](N2CCc3c(Cl)c(-c4ccc(F)c5sc(N)c(C#N)c45)c(F)c4nc(N5CC(C)(N6CCCC6)C5)cc2c34)C[C@H]1C(F)(F)F] | 2-amino-4-(7-chloro-9-fluoro-2-(3- methyl-3-(pyrrolidin-1-yl)azetidin-1-yl)-4- ((3R,5S)-1-(methyl-d₃)-2-oxo-5- (trifluoromethyl)pyrrolidin-3-yl)-5,6- dihydro-4H-benzo[de] [1,6]naphthyridin- 8-yl)-7-fluorobenzo[b]thiophene-3- carbonitrile | 719.2 |

The compounds of Examples 49-51 of Table 7 are understood to comprise atropisomers, i.e.,

### Example 49a and 49b

¹H NMR (500 MHz, DMSO) b 8.03 (s, 2H), 7.20 (dd, J=8.4, 5.3 Hz, 1H), 7.13 (t, J=8.9 Hz, 1H), 5.93 (s, 1H), 5.08 (t, J=9.3 Hz, 1H), 4.63 (tt, J=10.2, 5.1 Hz, 1H), 4.34 (d, J=9.9 Hz, 1H), 4.28 (d, J=9.8 Hz, 1H), 4.12-4.03 (m, 2H), 3.44 (dt, J=12.5, 6.4 Hz, 1H), 3.25 (dt, J=12.8, 6.5 Hz, 1H), 3.08 (d, J=12.9 Hz, 1H), 2.78 (s, 6H), 2.64-2.53 (m, 2H), 1.64 (s, 3H). The compounds of Example 49 are understood to comprise atropisomers, i.e., (Rₐ)-2-amino-4-(7-chloro-2-(3-(dimethylamino)-3-methylazetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)-3-methylazetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-(3-(dimethylamino)-3-methylazetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)-3-methylazetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-2-(3-(dimethylamino)-3-methylazetidin-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(3-(dimethylamino)-3-methylazetidin-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile. (Rₐ)-2-amino-4-(7-chloro-2-(3-(dimethylamino)-3-methylazetidin-1-yl)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)-3-methylazetidin-1-yl)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 50a and 50b

(Rₐ)-2-amino-4-(7-chloro-9-fluoro-4-((3R,5S)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile. are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-4-((3R,5R)-1-(methyl-d₃)-2-oxo-5-(trifluoromethyl)pyrrolidin-3-yl)-2-(3-(S-methylsulfonimidoyl)azetidin-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5R)-1-(methyl-d₃)-2-oxo-5-(trifluoromethyl)pyrrolidin-3-yl)-2-(3-(S-methylsulfonimidoyl)azetidin-1-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5S)-1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile.

### Example 51

The compound of Example 51 is understood to comprise a mixture of atropisomers, i.e., (Rₐ)-2-amino-4-(7-chloro-9-fluoro-2-(3-methyl-3-(pyrrolidin-1-yl)azetidin-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-methyl-3-(pyrrolidin-1-yl)azetidin-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-(3-methyl-3-(pyrrolidin-1-yl)azetidin-1-yl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-methyl-3-(pyrrolidin-1-yl)azetidin-1-yl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-2-(3-methyl-3-(pyrrolidin-1-yl)azetidin-1-yl)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(3-methyl-3-(pyrrolidin-1-yl)azetidin-1-yl)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(3-methyl-3-(pyrrolidin-1-yl)azetidin-1-yl)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-methyl-3-(pyrrolidin-1-yl)azetidin-1-yl)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 52a and 52b. 2-Amino-4-(7-chloro-9-fluoro-2-(((2R,7aS)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

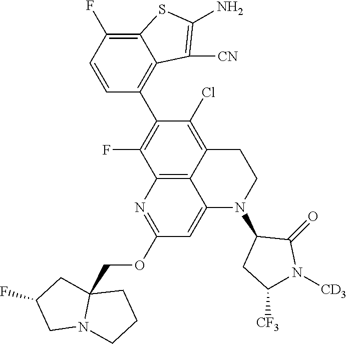 [SMILES: [2H]C([2H])([2H])N1C(=O)[C@H](N2CCc3c(Cl)c(-c4ccc(F)c5sc(N)c(C#N)c45)c(F)c4nc(OC[C@@]56CCCN5C[C@H](F)C6)cc2c34)C[C@H]1C(F)(F)F]

### Step 1. (3R,5S)-3-(8-Bromo-7-chloro-9-fluoro-2-(((2R,7aS)-2-fluorotetrahydro-1H-pyrrolizin-7a(5H)-yl)methoxy)-5,6-dihydro-4H-benzo[de][1,6]naphthyridin-4-yl)-1-(methyl-d₃)-5-(trifluoromethyl)pyrrolidin-2-one

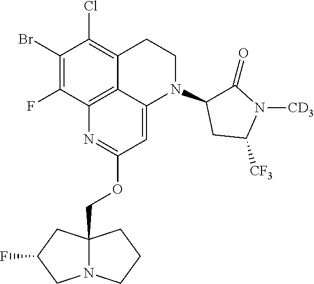 [SMILES: [2H]C([2H])([2H])N1C(=O)[C@H](N2CCc3c(Cl)c(Br)c(F)c4nc(OC[C@@]56CCCN5C[C@H](F)C6)cc2c34)C[C@H]1C(F)(F)F]

In a 1 dram vial, Intermediate 13 (40 mg, 0.067 mmol), CuI (4.5 mg, 0.024 mmol), Cs₂CO₃ (44 mg, 0.134 mmol), ((2R,7aS)-2-fluorotetrahydro-1H-pyrrolizin-7a(5H)-yl)methanol (53 mg, 0.34 mmol) and 3,4,7,8-Tetramethyl-1,10-phenanthroline (6 mg, 0.027 mmol) were dissolved in toluene (500 μL) under N₂. The mixture was stirred at 110° C. for 3 h, then diluted with MeCN, filtered and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₂₅H₂₃D₃BrClF₅N₄O₂(M+H)⁺ m/z=626.1, 628.1; found 626.1, 628.1.

### Step 2. 2-Amino-4-(7-chloro-9-fluoro-2-(((2R,7aS)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-9-fluoro-2-(((2R,7aS)-2-fluorotetrahydro-1H-pyrrolizin-7a(5H)-yl)methoxy)-5,6-dihydro-4H-benzo[de][1,6]naphthyridin-4-yl)-1-(methyl-d₃)-5-(trifluoromethyl)pyrrolidin-2-one (16 mg, 0.026 mmol), tert-butyl (3-cyano-4-(5,5-dimethyl-1,3,2-dioxaborinan-2-yl)-7-fluorobenzo[b]thiophen-2-yl)carbamate (26 mg, 0.064 mmol), dichloro [bis(diphenylphosphinophenyl)ether]palladium(II) (4 mg, 6.4 μmol), and K₂CO₃ (14 mg, 0.102 mmol) were dissolved in 1,4-dioxane (200 μL) under N₂. The mixture was stirred at 100° C. for 1 h, then diluted with MeCN and filtered. The filtrate was concentrated and dissolved in 1:1 DCM/TFA (1 mL). After stirring at r.t. for 30 min., the mixture was concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 52a. Diastereomer 1. Peak 1. LCMS calc. for C₃₄H₂₇D₃ClF₆N₆O₂S (M+H)⁺ m/z=738.2; found 738.2. This is the potent peak.

Example 52b. Diastereomer 2. Peak 2. LCMS calc. for C₃₄H₂₇D₃ClF₆N₆O₂S (M+H)⁺ m/z=738.2; found 738.2.

The compounds are understood to comprise atropisomers, i.e., (Rₐ)-2-amino-4-(7-chloro-9-fluoro-2-(((2R,7aS)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2R,7aS)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-(((2R,7aS)-2-fluorotetrahydro-1H-pyrrolizin-7a(5H)-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2R,7aS)-2-fluorotetrahydro-1H-pyrrolizin-7a(5H)-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-2-(((2R,7aS)-2-fluorotetrahydro-1H-pyrrolizin-7a(5H)-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,7aS)-2-fluorotetrahydro-1H-pyrrolizin-7a(5H)-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,7aS)-2-fluorotetrahydro-1H-pyrrolizin-7a(5H)-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2R,7aS)-2-fluorotetrahydro-1H-pyrrolizin-7a(5H)-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,7aR)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,7aR)-2-fluorotetrahydro-1H-pyrrolizin-7a(5H)-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,7aR)-2-fluorotetrahydro-1H-pyrrolizin-7a(5H)-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2S,7aR)-2-fluorotetrahydro-1H-pyrrolizin-7a(5H)-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridine-8-yl)-7-fluorobenzo[b]thiophene-3-carbonitrile; (Rₐ)-2-amino-4-(7-chloro-9-fluoro-2-(((2S,7aR)-2-fluorotetrahydro-1H-pyrrolizin-7a(5H)-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,7aR)-2-fluorotetrahydro-1H-pyrrolizin-7a(5H)-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,7aR)-2-fluorotetrahydro-1H-pyrrolizin-7a(5H)-yl)methoxy)-4-((3S,5S)-1-(methyl-d₃)-2-oxo-5-(trifluoromethyl)(pyrrolidin-3-yl)-5,6-dihydro-4H-benzo[de][1,6]naphthyridin-8-yl)-7-fluorobenzo[b]thiophene-3-carbonitrile; and (Sₐ)-2amino-4-(7-chloro-9-fluoro-2-(((2S,7aR)-2-fluorotetrahydro-1H-pyrrolizin-7a(5H)-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Examples 53-55

Compounds in Table 8 were prepared according to the general procedure described for Example 52, using the appropriate alcohol starting material.

TABLE 8

| # | Structure | Name | [M + H] |
|---|---|---|---|
| 53a/b | 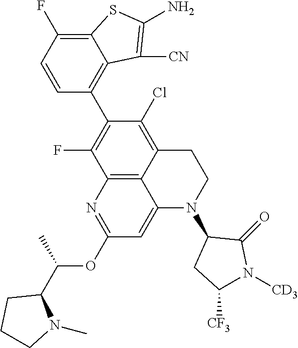 [SMILES: [2H]C([2H])([2H])N1C(=O)[C@H](N2CCc3c(Cl)c(-c4ccc(F)c5sc(N)c(C#N)c45)c(F)c4nc(O[C@@H](C)[C@@H]5CCCN5C)cc2c34)C[C@H]1C(F)(F)F] | 2-amino-4-(7-chloro-9-fluoro-4-((3R,5S)- 1-(methyl-d₃)-2-oxo-5- (trifluoromethyl)pyrrolidin-3-yl)-2-((S)-1- ((S)-1-methylpyrrolidin-2-yl)ethoxy)-5,6- dihydro-4H-benzo[de][1,6]naphthyridin- 8-yl)-7-fluorobenzo[b]thiophene-3- carbonitrile | 708.2 |
| 54a/b | 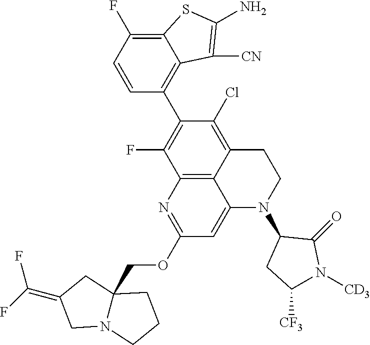 [SMILES: [2H]C([2H])([2H])N1C(=O)[C@H](N2CCc3c(Cl)c(-c4ccc(F)c5sc(N)c(C#N)c45)c(F)c4nc(OC[C@@]56CCCN5CC(=C(F)F)C6)cc2c34)C[C@H]1C(F)(F)F] | 2-amino-4-(7-chloro-2-(((S)-2- (difluoromethylene)tetrahydro-1H- pyrrolizin-7a(5H)-yl)methoxy)-9-fluoro-4- ((3R,5S)-1-(methyl-d₃)-2-oxo-5- (trifluoromethyl)pyrrolidin-3-yl)-5,6- dihydro-4H-benzo[de][1,6]naphthyridin- 8-yl)-7-fluorobenzo[b]thiophene-3- carbonitrile | 768.2 |
| 55a/b | 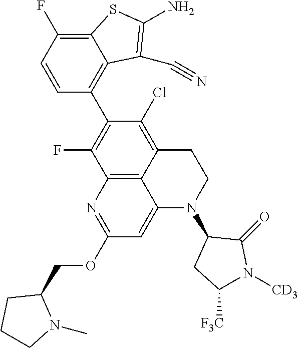 [SMILES: [2H]C([2H])([2H])N1C(=O)[C@H](N2CCc3c(Cl)c(-c4ccc(F)c5sc(N)c(C#N)c45)c(F)c4nc(OC[C@@H]5CCCN5C)cc2c34)C[C@H]1C(F)(F)F] | 2-amino-4-(7-chloro-9-fluoro-4-((3R,5S)- 1-(methyl-d₃)-2-oxo-5- (trifluoromethyl)pyrrolidin-3-yl)-2-(((S)-1- methylpyrrolidin-2-yl)methoxy)-5,6- dihydro-4H-benzo[de][1,6]naphthyridin- 8-yl)-7-fluorobenzo[b]thiophene-3- carbonitrile | 694.2 |
| 56a/b | 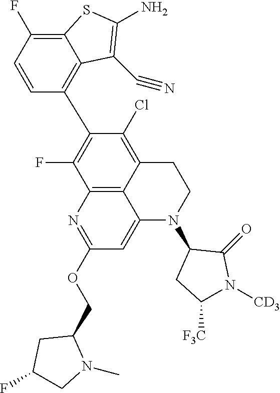 [SMILES: [2H]C([2H])([2H])N1C(=O)[C@H](N2CCc3c(Cl)c(-c4ccc(F)c5sc(N)c(C#N)c45)c(F)c4nc(OC[C@@H]5C[C@@H](F)CN5C)cc2c34)C[C@H]1C(F)(F)F] | 2-amino-4-(7-chloro-9-fluoro-2- (((2S,4R)-4-fluoro-1-methylpyrrolidin-2- yl)methoxy)-4-((3R,5S)-1-(methyl-d₃)-2- oxo-5-(trifluoromethyl)pyrrolidin-3-yl)- 5,6-dihydro-4H- benzo[de][1,6]naphthyridin-8-yl)-7- fluorobenzo[b]thiophene-3-carbonitrile | 712.2 |

The compounds of Examples 53-56 of Table 8 are understood to comprise atropisomers, i.e.,

### Examples 53a and 53b

The compounds of Example 53 are understood to comprise atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-4-((3R,5R)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile. (Rₐ)-2-amino-4-(7-chloro-9-fluoro-4-((3S,5S)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-1-(methyl-d₃)-2-oxo-5-(trifluoromethyl)pyrrolidin-3-yl)-2-((R)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-((S)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-1-(methyl-d₃)-2-oxo-5-(trifluoromethyl)pyrrolidin-3-yl)-2-((R)-1-((S)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5S)-1-(methyl-d₃)-2-oxo-5-(trifluoromethyl)pyrrolidin-3-yl)-2-((S)-1-((R)-1-methylpyrrolidin-2-yl)ethoxy)-5,6-dihydro-4H-benzo[de][1,6]naphthyridin-8-yl)-7-fluorobenzo[b]thiophene-3-carbonitrile.

### Examples 54a and 54b

(Rₐ)-2-amino-4-(7-chloro-2-(((S)-2-(difluoromethylene)tetrahydro-1H-pyrrolizin-7a(5H)-yl)methoxy)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and
(Sₐ)-2-amino-4-(7-chloro-2-(((S)-2-(difluoromethylene)tetrahydro-1H-pyrrolizin-7a(5H)-yl)methoxy)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e.,
(Rₐ)-2-amino-4-(7-chloro-2-(((S)-2-(difluoromethylene)tetrahydro-1H-pyrrolizin-7a(5H)-yl)methoxy)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(((S)-2-(difluoromethylene)tetrahydro-1H-pyrrolizin-7a(5H)-yl)methoxy)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing 15 appropriate starting materials, i.e., (Rₐ)-2-amino-4-(7-chloro-2-(((S)-2-(difluoromethylene)tetrahydro-1H-pyrrolizin-7a(5H)-yl)methoxy)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(((S)-2-(difluoromethylene)tetrahydro-1H-pyrrolizin-7a(5H)-yl)methoxy)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(((S)-2-(difluoromethylene)tetrahydro-1H-pyrrolizin-7a(5H)-yl)methoxy)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(((S)-2-(difluoromethylene)tetrahydro-1H-pyrrolizin-7a(5H)-yl)methoxy)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(((R)-2-(difluoromethylene)tetrahydro-1H-pyrrolizin-7a(5H)-yl)methoxy)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(((R)-2-(difluoromethylene)tetrahydro-1H-pyrrolizin-7a(5H)-yl)methoxy)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(((R)-2-(difluoromethylene)tetrahydro-1H-pyrrolizin-7a(5H)-yl)methoxy)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(((R)-2-(difluoromethylene)tetrahydro-1H-pyrrolizin-7a(5H)-yl)methoxy)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(((R)-2-(difluoromethylene)tetrahydro-1H-pyrrolizin-7a(5H)-yl)methoxy)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-(((R)-2-(difluoromethylene)tetrahydro-1H-pyrrolizin-7a(5H)-yl)methoxy)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(((R)-2-(difluoromethylene)tetrahydro-1H-pyrrolizin-7a(5H)-yl)methoxy)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(((R)-2-(difluoromethylene)tetrahydro-1H-pyrrolizin-7a(5H)-yl)methoxy)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Examples 55a and 55b

(Rₐ)-2-amino-4-(7-chloro-9-fluoro-4-((3R,5S)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-4-((3R,5R)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5R)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile. (Rₐ)-2-amino-4-(7-chloro-9-fluoro-4-((3S,5S)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-1-(methyl-d₃)-2-oxo-5-(trifluoromethyl)pyrrolidin-3-yl)-2-(((S)-1-methylpyrrolidin-2-yl)methoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5R)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile. (Rₐ)-2-amino-4-(7-chloro-9-fluoro-4-((3S,5S)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5S)-1-(methyl-d₃)-2-oxo-5-(trifluoromethyl)pyrrolidin-3-yl)-2-(((R)-1-methylpyrrolidin-2-yl)methoxy)-5,6-dihydro-4H-benzo[de][1,6]naphthyridin-8-yl)-7-fluorobenzo[b]thiophene-3-carbonitrile.

### Examples 56

The compound of Example 56 is understood to comprise a mixture of atropisomers, i.e.,
(Rₐ)-2-amino-4-(7-chloro-9-fluoro-2-(((2S,4R)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and
(Sₐ)-2-amino-4-(7-chloro-9-fluoro-2-(((2S,4R)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e.,
(Rₐ)-2-amino-4-(7-chloro-9-fluoro-2-(((2S,4R)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and
(Sₐ)-2-amino-4-(7-chloro-9-fluoro-2-(((2S,4R)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing 10 appropriate starting materials, i.e., (Rₐ)-2-amino-4-(7-chloro-9-fluoro-2-(((2S,4R)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2S,4R)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,4R)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,4R)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4R)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4R)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4R)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4R)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4R)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4R)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4R)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4R)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,4S)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,4S)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,4S)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,4S)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,4S)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,4S)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2S,4S)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2S,4S)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4S)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4S)-4-fluoro-1-methylpyrrolidin-2-yl)methoxy)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4S)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4S)-4-fluoro-1-methylpyrrolidin-2-yl)methoxy)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4S)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(((2R,4S)-4-fluoro-1-methylpyrrolidin-2-yl)methoxy)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(((2R,4S)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(((2R,4S)-4-fluoro-1-methylpyrrolidin-2-yl)methoxy)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 57a and 57b. 2-Amino-4-(7-chloro-9-fluoro-4-((3R,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile

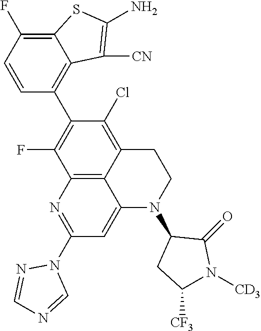 [SMILES: [2H]C([2H])([2H])N1C(=O)[C@H](N2CCc3c(Cl)c(-c4ccc(F)c5sc(N)c(C#N)c45)c(F)c4nc(-n5cncn5)cc2c34)C[C@H]1C(F)(F)F]

### Step 1. (3R,5S)-3-(8-Bromo-7-chloro-9-fluoro-2-(1H-1,2,4-triazol-1-yl)-5,6-dihydro-4H-benzo[de][1,6]naphthyridin-4-yl)-1-(methyl-d₃)-5-(trifluoromethyl)pyrrolidin-2-one

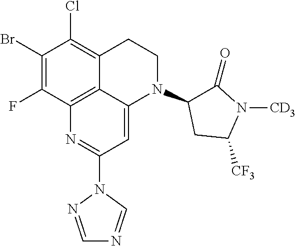 [SMILES: [2H]C([2H])([2H])N1C(=O)[C@H](N2CCc3c(Cl)c(Br)c(F)c4nc(-n5cncn5)cc2c34)C[C@H]1C(F)(F)F]

In a 1 dram vial, Intermediate 13 (50 mg, 0.084 mmol), 1H-1,2,4-triazole (116 mg, 1.68 mmol), L-proline (17 mg, 0.147 mmol), and CuI (16 mg, 0.084 mmol) and K₂CO₃ (116 mg, 0.840 mmol) were dissolved in DMSO (840 μL) under N₂. The mixture was heated at 85° C. for 15 min. The reaction was quenched with water and extracted with EtOAc. The organic layer was washed with water and brine, dried over MgSO₄ and concentrated. The crude product was purified by prep. SFC (4-24% MeOH in CO₂, 2-PIC column, 140 mL/min. flow rate) to provide the title compound. LCMS calc. for C₁₉H₁₂D₃BrClF₄N₆O (M+H)⁺ m/z=536.0, 538.0; found 536.0, 538.0.

### Step 2. 2-Amino-4-(7-chloro-9-fluoro-4-((3R,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-9-fluoro-2-(1H-1,2,4-triazol-1-yl)-5,6-dihydro-4H-benzo[de][1,6]naphthyridin-4-yl)-1-(methyl-d₃)-5-(trifluoromethyl)pyrrolidin-2-one (14 mg, 0.026 mmol), tert-butyl (3-cyano-4-(5,5-dimethyl-1,3,2-dioxaborinan-2-yl)-7-fluorobenzo[b]thiophen-2-yl)carbamate (26 mg, 0.065 mmol), dichloro[bis(diphenylphosphinophenyl)ether]palladium(II) (6 mg, 7.8 μmol), and K₂CO₃ (11 mg, 0.078 mmol) were dissolved in 1,4-dioxane (200 μL) under N₂. The mixture was heated at 100° C. for 1 h, then diluted with MeCN and filtered. The filtrate was concentrated and the residue was dissolved in 1:1 DCM/TFA (1 mL) and stirred at r.t. for 30 min., then concentrated. The residue was dissolved in MeCN and purified by prep. HPLC (XBRIDGE® Cl₈ column, eluting with a gradient of MeCN/water containing 0.1% NH₄OH, at flow rate of 60 mL/min.) to afford the title compound as two peaks.

Example 57a. Diastereomer 1. Peak 1. LCMS calc. for C₂₈H₁₆D₃ClF₅N₈₀S (M+H)⁺ m/z=648.1; found 648.1. This is the potent peak.

Example 57b. Diastereomer 2. Peak 2. LCMS calc. for C₂₈H₁₆D₃ClF₅N₈₀S (M+H)⁺ m/z=648.1; found 648.1.

The compounds are understood to comprise atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

Further isomers are prepared by using modifications to the procedure and employing appropriate starting materials, i.e., (Rₐ)-2-amino-4-(7-chloro-9-fluoro-4-((3R,5R)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5R)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5S)-1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile.

### Example 58a and 58b. 8-(2-Amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide

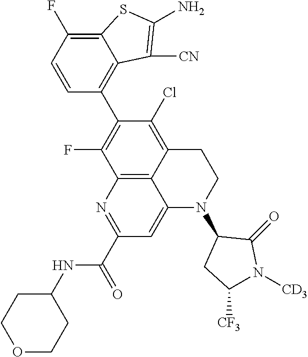 [SMILES: [2H]C([2H])([2H])N1C(=O)[C@H](N2CCc3c(Cl)c(-c4ccc(F)c5sc(N)c(C#N)c45)c(F)c4nc(C(=O)NC5CCOCC5)cc2c34)C[C@H]1C(F)(F)F]

### Step 1. Methyl 8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carboxylate

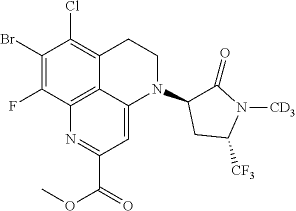 [SMILES: [2H]C([2H])([2H])N1C(=O)[C@H](N2CCc3c(Cl)c(Br)c(F)c4nc(C(=O)OC)cc2c34)C[C@H]1C(F)(F)F]

Under an atmosphere of argon, a solution of Intermediate 13 (1.09 g, 1.830 mmol), 5 and (dppf)PdCl₂ (0.149 g, 0.183 mmol) in 1,4-dioxane (9.15 mL)/MeOH (9.15 mL) was degassed for 10 min. with CO gas. Then TEA (1.28 mL, 9.15 mmol) was added and the solution was stirred at 60° C. under a CO atmosphere for 4 h. The mixture was filtered through a pad of diatomaceous earth. The filtrate was concentrated and the crude residue was purified by FCC (20% EtOAc in hexanes) to provide the title compound (780 mg, 81%). LCMS calc. for C₁₅H₁₃D₃BrClF₄N₃O₃ (M+H)⁺ m/z=527.0, 529.0; found 527.0, 529.0.

### Step 2. 8-Bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carboxylic acid

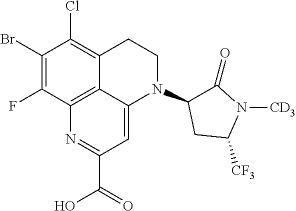 [SMILES: [2H]C([2H])([2H])N1C(=O)[C@H](N2CCc3c(Cl)c(Br)c(F)c4nc(C(=O)O)cc2c34)C[C@H]1C(F)(F)F]

To solution of methyl 8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carboxylate (780 mg, 1.478 mmol) in THF (5 mL) was added NaOH (1 N, 3.0 mL, 3.0 mmol) at 50° C. After stirring for 1 h at this temperature, the mixture was diluted with water and EtOAc. The pH was adjusted to 7 using 1 M HCl. The organic layer was separated, washed with brine, dried with Na₂SO₄ and concentrated. The crude product was used in the next step without 20 further purification. LCMS calc. for C₁₃H₁₁D₃BrClF₄N₃O₃ (M+H)⁺ m/z=513.0, 515.0; found 513.0, 515.0.

### Step 3. 8-Bromo-7-chloro-9-fluoro-4-((3R,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide

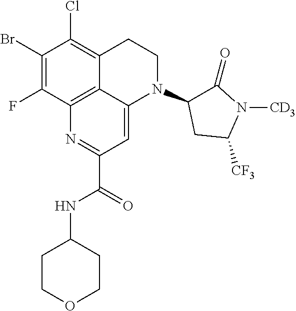 [SMILES: [2H]C([2H])([2H])N1C(=O)[C@H](N2CCc3c(Cl)c(Br)c(F)c4nc(C(=O)NC5CCOCC5)cc2c34)C[C@H]1C(F)(F)F]

To a solution of 8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carboxylic acid (21 mg, 0.041 mmol) in DMF (150 μL) were added HATU (19 mg, 0.049 mmol), tetrahydro-2H-pyran-4-amine (4 mg, 0.041 mmol) and DIPEA (26 μL, 0.147 mmol). The mixture was stirred at r.t. for 1 h, then diluted with MeCN and purified by prep. HPLC (XBRIDGE® C₁₈ column, eluting with a gradient of MeCN/water containing 0.1% TFA, at flow rate of 60 mL/min.) to afford the title compound. LCMS calc. for C₂₃H₂₀D₃BrClF₄N₄O₃ (M+H)⁺ m/z=596.1, 598.1; found 596.1, 598.1.

### Step 4. 8-(2-Amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide

A mixture of 8-bromo-7-chloro-9-fluoro-4-((3R,5S)-1-(methyl-d₃)-2-oxo-5-(trifluoromethyl) pyrrolidin-3-yl)-N-(tetrahydro-2H-pyran-4-yl)-5,6-dihydro-4H-benzo[de][1,6]naphthyridine-2-carboxamide (18 mg, 0.030 mmol), tert-butyl (3-cyano-4-(5,5-dimethyl-1,3,2-dioxaborinan-2-yl)-7-fluorobenzo[b]thiophen-2-yl)carbamate (24 mg, 0.060 mmol), (dppf)PdCl₂ (5 mg, 6.0 μmol) and K₂CO₃ (21 mg, 0.151 mmol) in 1,4-dioxane (1 mL) was stirred at 90° C. for 80 min. The mixture was filtered and concentrated. The crude was treated with 1:1 DCM/TFA (1 mL) at r.t. for 50 min. The volatiles were removed in vacuo. The residue was diluted with MeCN and purified by prep. HPLC (XBRIDGE® C₁₈ column, eluting with a gradient of MeCN/water containing 0.1% TFA, at flow rate of 60 mL/min.) to afford the title compound as two peaks.

Example 58a. Diastereomer 1. Peak 1. LCMS calc. for C₃₂H₂₄D₃ClF₅N₆O₃S (M+H)⁺ m/z=708.2; found 708.2. This is the potent peak.

Example 58b. Diastereomer 2. Peak 2. LCMS calc. for C₃₂H₂₄D₃ClF₅N₆O₃S (M+H)⁺ m/z=708.2; found 708.2.

The compounds are understood to comprise atropisomers, i.e., 8-(Rₐ)-(2-amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide.

The alternative isomers, i.e., 8-(Rₐ)-(2-amino-3-cyano-7-fluorobenzo[b]thiophen-4-yl)-7-chloro-9-fluoro-4-((3S,5R)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-4-((3S,5R)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; are prepared by an analogous procedure using the alternative diastereomer of Intermediate 13 as starting material.

Further isomers are prepared by using modifications to the procedure and employing appropriate starting materials, i.e., 8-(Rₐ)-(2-amino-3-cyano-7-fluorobenzo[b]thiophen-4-yl)-7-chloro-9-fluoro-4-((3R,5R)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; 8-(Sₐ)-(2-amino-3-cyano-7-fluorobenzo[b]thiophen-4-yl)-7-chloro-9-fluoro-4-((3R,5R)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; 8-(Rₐ)-(2-amino-3-cyano-7-fluorobenzo[b]thiophen-4-yl)-7-chloro-9-fluoro-4-((3S,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide; and 8-(Sₐ)-(2-amino-3-cyano-7-fluorobenzo[b]thiophen-4-yl)-7-chloro-9-fluoro-4-((3S,5S)-1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide.

### Example 59a and 59b. 8-(2-Amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile

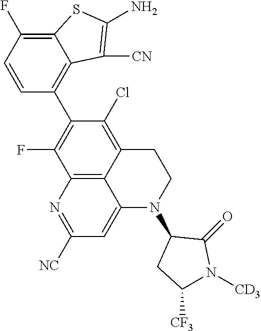 [SMILES: [2H]C([2H])([2H])N1C(=O)[C@H](N2CCc3c(Cl)c(-c4ccc(F)c5sc(N)c(C#N)c45)c(F)c4nc(C#N)cc2c34)C[C@H]1C(F)(F)F]

### Step 1. 8-Bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile

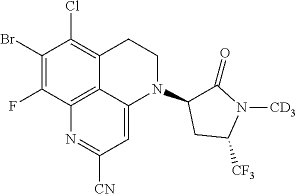 [SMILES: [2H]C([2H])([2H])N1C(=O)[C@H](N2CCc3c(Cl)c(Br)c(F)c4nc(C#N)cc2c34)C[C@H]1C(F)(F)F]

To a 8 mL vial, were added CuCN (12.03 mg, 0.134 mmol), Intermediate 13 (40 mg, 0.067 mmol) and NMP (400 μL) under N₂. The mixture was warmed to 80° C. and stirred for 4 h, then cooled to r.t. The reaction was quenched with sat. aq. NaHCO₃ and diluted with EtOAc. The mixture was filtered through diatomaceous earth and the filtrate was phase separated. The organic layer was washed with brine, dried over Na₂SO₄ and concentrated. The crude product was purified by FCC (0-100% EtOAc in hexanes) to provide the title compound (26 mg, 79%). LCMS calc. for C₁₈H₁₀D₃BrClF₄N₄O (M+H)⁺ m/z=494.0, 496.0; found 494.0, 496.0.

### Step 2. 8-(2-Amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile

To the mixture of dichloro[bis(2-(diphenylphosphino)phenyl)ether]palladium(II) (10 mg, 0.013 mmol), Cs₂CO₃ (34 mg, 0.106 mmol), tert-butyl (3-cyano-4-(5,5-dimethyl-1,3,2-dioxaborinan-2-yl)-7-fluorobenzo[b]thiophen-2-yl)carbamate (42.8 mg, 0.106 mmol), and 8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile (26 mg, 0.053 mmol) was added 1,4-dioxane (530 μL) under N₂. The mixture was heated at 95° C. for 2 h. The mixture was diluted with EtOAc and filtered. The filtrate was concentrated, then purified by FCC (0-100% EtOAc in hexanes). The intermediate was dissolved in 1:1 DCM/TFA (1 mL) and the mixture was stirred at r.t. for 30 min., then concentrated. The residue was dissolved in MeCN and purified by prep. HPLC (XBRIDGE© C₁₈ column, eluting with a gradient of MeCN/water containing 0.1% NH₄OH, at flow rate of 60 mL/min.) to afford the title compound as two peaks.

Example 59a. Diastereomer 1. Peak 1. LCMS calc. for C₂₇H₁₄D₃ClF₅N₆OS (M+H)⁺ m/z=606.1; found 606.1. This is the potent peak.

Example 59b. Diastereomer 2. Peak 2. LCMS calc. for C₂₇H₁₄D₃ClF₅N₆OS (M+H)⁺ m/z=606.1; found 606.1.

The compounds are understood to comprise atropisomers, i.e., 8-(Rₐ)-(2-amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile; and 8-(Sₐ)-(2-amino-3-cyano-7-fluorobenzo[b]thiophen-4-yl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbonitrile. The alternative isomers, i.e., 8-(Rₐ)-(2-amino-3-cyano-7-fluorobenzo[b]thiophen-4-yl)-7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridine-2-carbonitrile; and 8-(Sₐ)-(2-amino-3-cyano-7-fluorobenzo[b]thiophen-4-yl)-7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridine-2-carbonitrile; are prepared by an analogous procedure using the alternative diastereomer of Intermediate 13 as starting material.

Further isomers are prepared by using modifications to the procedure and employing appropriate starting materials, i.e., 8-(Rₐ)-(2-amino-3-cyano-7-fluorobenzo[b]thiophen-4-yl)-7-chloro-9-fluoro-4-((3R,5R)-1-(methyl-d₃)-2-oxo-5-(trifluoromethyl)pyrrolidin-3-yl)-5,6-dihydro-4H-benzo[de][1,6]naphthyridine-2-carbonitrile; 8-(Sₐ)-(2-amino-3-cyano-7-fluorobenzo[b]thiophen-4-yl)-7-chloro-9-fluoro-4-((3R,5R)-1-(methyl-d₃)-2-oxo-5-(trifluoromethyl)pyrrolidin-3-yl)-5,6-dihydro-4H-benzo[de][1,6]naphthyridine-2-carbonitrile. 8-(Rₐ)-(2-amino-3-cyano-7-fluorobenzo[b]thiophen-4-yl)-7-chloro-9-fluoro-4-((3S,5S)-1-(methyl-d₃)-2-oxo-5-(trifluoromethyl)pyrrolidin-3-yl)-5,6-dihydro-4H-benzo[de][1,6]naphthyridine-2-carbonitrile; and 8-(Sₐ)-(2-amino-3-cyano-7-fluorobenzo[b]thiophen-4-yl)-7-chloro-9-fluoro-4-((3S,5S)-1-(methyl-d₃)-2-oxo-5-(trifluoromethyl)pyrrolidin-3-yl)-5,6-dihydro-4H-benzo[de][1,6]naphthyridine-2-carbonitrile.

### Example 60a and 60b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile

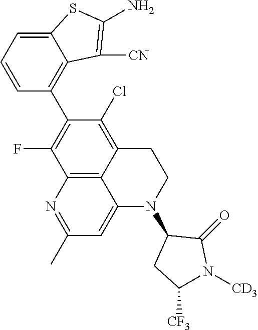 [SMILES: [2H]C([2H])([2H])N1C(=O)[C@H](N2CCc3c(Cl)c(-c4cccc5sc(N)c(C#N)c45)c(F)c4nc(C)cc2c34)C[C@H]1C(F)(F)F]

To a mixture of Intermediate 12 (20 mg, 0.041 mmol), tert-butyl (3-cyano-4-(5,5-dimethyl-1,3,2-dioxaborinan-2-yl)benzo[b]thiophen-2-yl)carbamate (48 mg, 0.12 mmol), K₂CO₃ (17 mg, 0.124 mmol) and Pd-117 (6 mg, 0.008 mmol) was added 1,4-dioxane (400 μL) and the reaction flask was evacuated, back filled with N₂, then stirred at 100° C. overnight. The mixture was diluted with MeCN and filtered. The filtrate was concentrated and the residue was dissolved in 1:1 TFA/DCM (1 mL) and stirred for 30 min. at r.t. The solution was concentrated and the crude product was purified by prep. HPLC (XBRIDGE® C₁₈ column, eluting with a gradient of MeCN/water containing 0.1% NH₄OH, at flow rate of 60 mL/min.) to afford the title compound as two peaks.

Example 60a. Diastereomer 1. Peak 1. LCMS calc. for C₂₇H₁₈D₃ClF₄N₅OS (M+H)⁺ m/z=577.1; found 577.1. This is the potent peak.

Example 60b. Diastereomer 2. Peak 2. LCMS calc. for C₂₇H₁₈D₃ClF₄N₅OS (M+H)⁺ m/z=577.1; found 577.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; are prepared by an analogous procedure using the alternative diastereomer of Intermediate 12 as starting material.

Further isomers are prepared by using modifications to the procedure and employing appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile;

### Example 61a and 61b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile

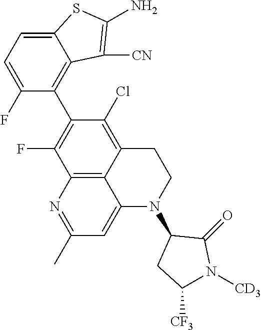 [SMILES: [2H]C([2H])([2H])N1C(=O)[C@H](N2CCc3c(Cl)c(-c4c(F)ccc5sc(N)c(C#N)c45)c(F)c4nc(C)cc2c34)C[C@H]1C(F)(F)F]

### Step 1. tert-Butyl (4-bromo-3-cyano-5-fluorobenzo[b]thiophen-2-yl)carbamate

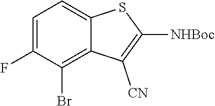 [SMILES: CC(C)(C)OC(=O)Nc1sc2ccc(F)c(Br)c2c1C#N]

To a suspension of tert-butyl (4-bromo-5-fluorobenzo[b]thiophen-2-yl)carbamate (330 mg, 0.953 mmol) in MeCN (6 mL) at 0° C. was added sulfurisocyanatidic chloride (249 μL, 2.86 mmol) dropwise. The solution was stirred at 0° C. for 20 min. After that, DMF (1.2 mL) was added dropwise. The mixture was stirred at r.t. for 1 h, then quenched with sat. ammonium chloride. The resulting solid was collected by filtration and used in the next step without further purification (260 mg, 73%). LCMS calc. for C₁₄H₁₃BrFN₂O₂S (M+H)⁺ m/z=371.0, 373.0; found 371.0, 373.0.

### Step 2. tert-Butyl (4-bromo-3-cyano-5-fluorobenzo[b]thiophen-2-yl)((2-(trimethylsilyl) ethoxy)methyl)carbamate

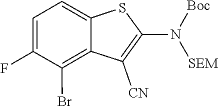 [SMILES: CC(C)(C)OC(=O)N(COCC[Si](C)(C)C)c1sc2ccc(F)c(Br)c2c1C#N]

To a solution of tert-butyl (4-bromo-3-cyano-5-fluorobenzo[b]thiophen-2-yl)carbamate (250 mg, 0.67 mmol) in DMF (3.4 mL) at 0° C. was added NaH (60% w/w mineral oil, 30 mg, 0.74 mmol) and the mixture was stirred at r.t. for 30 min., then recooled to 0° C. SEMCl (160 μL, 0.81 mmol) was added and the mixture was warmed to r.t., then quenched with sat. aq. NaHCO₃. The mixture was extracted with EtOAc. The organic layer was washed with water and brine, dried over Na₂SO₄ and concentrated. The crude product was purified by FCC (0-40% EtOAc in hexanes) to provide the title compound (220 mg, 65%). LCMS calc. for C₂₀H₂₇BrFN₂O₃SSi (M+H)⁺ m/z=501.1, 503.1; found 501.1, 503.1.

### Step 3. tert-Butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-5-fluorobenzo[b]thiophen-2-yl)((2-(trimethylsilyl)ethoxy)methyl)carbamate

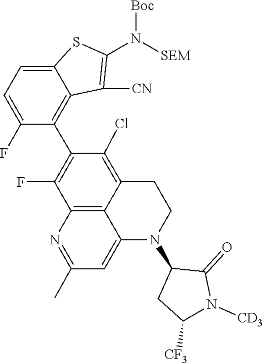 [SMILES: [2H]C([2H])([2H])N1C(=O)[C@H](N2CCc3c(Cl)c(-c4c(F)ccc5sc(N(COCC[Si](C)(C)C)C(=O)OC(C)(C)C)c(C#N)c45)c(F)c4nc(C)cc2c34)C[C@H]1C(F)(F)F]

To a solution of Intermediate 12 (60 mg, 0.124 mmol) in THF (766 μL) at −78° C. was added iPrMgCl·LiCl (1.3 M in THF, 200 μL, 0.260 mmol) dropwise. After stirring for 45 min. at −78° C., ZnCl₂ (1.9 M in 2-MeTHF, 137 μL, 0.260 mmol) was added dropwise. After stirring at −78° C. for an additional 10 min., the solution was warmed to r.t. for 30 min. The solution was transferred to a solution of tert-butyl (4-bromo-3-cyano-5-fluorobenzo[b]thiophen-2-yl)((2-(trimethylsilyl)ethoxy)methyl)carbamate (62 mg, 0.12 mmol) and RuPhos Pd G4 (20 mg, 0.025 mmol) in THF (613 μL) and the resulting mixture heated at 60° C. overnight. After cooling to r.t. the mixture was quenched with saturated NaHCO₃ and extracted with EtOAc. The organic layer was dried over Na₂SO₄ and concentrated. The crude product was purified by prep. HPLC (XBRIDGE® C₁₈ column, eluting with a gradient of MeCN/water containing 0.1% NH₄OH, at flow rate of 60 mL/min.) to afford the title compound (7 mg, 7%). LCMS calc. for C₃₃H₃₉D₃ClF₅N₅O₄SSi (M+H)⁺ m/z=825.2; found 825.2.

### Step 4. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile

To a solution of tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-5-fluorobenzo[b]thiophen-2-yl)((2-(trimethylsilyl)ethoxy)methyl)carbamate (7.0 mg, 8.48 μmol) in DCM (1 mL), was added TFA (1 mL) at r.t. After stirring for 2 h the mixture was concentrated, diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 61a. Diastereomer 1. Peak 1. LCMS calc. for C₂₇H₁₇D₃ClF₅N₅OS (M+H)⁺ m/z=595.1; found 595.1. This is the potent peak.

Example 61b. Diastereomer 2. Peak 2. LCMS calc. for C₂₇H₁₇D₃ClF₅N₅OS (M+H)⁺ m/z=595.1; found 595.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; are prepared by an analogous procedure using the alternative diastereomer of Intermediate 12 as starting material.

Further isomers are prepared by using modifications to the procedure and employing appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile.

### Example 62a and 62b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile

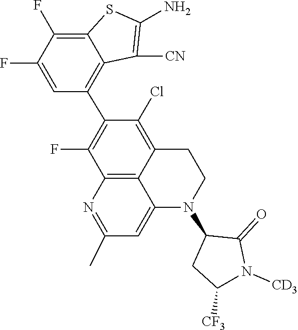 [SMILES: [2H]C([2H])([2H])N1C(=O)[C@H](N2CCc3c(Cl)c(-c4cc(F)c(F)c5sc(N)c(C#N)c45)c(F)c4nc(C)cc2c34)C[C@H]1C(F)(F)F]

### Step 1. 6-Bromo-2,3,4-trifluorobenzyl methanesulfonate

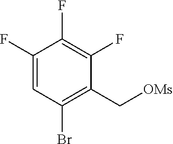 [SMILES: CS(=O)(=O)OCc1c(Br)cc(F)c(F)c1F]

To a solution of (6-bromo-2,3,4-trifluorophenyl)methanol (1.03 g, 4.27 mmol) in THF (11 mL) was added DIPEA (900 μL, 5.13 mmol) and the mixture was cooled to 0° C. Methanesulfonic anhydride (820 mg, 4.7 mmol) was then added and the mixture was allowed to warm to r.t. overnight. The mixture was diluted with EtOAc and washed with water. The organic layer was washed with water and brine, dried over Na₂SO₄ and concentrated. The crude product was used in the next step without further purification (1.36 g, >99%). LCMS calc. for C₃H₇BrF₃O₃S (M+H)⁺ m/z=319.0, 321.0; found 319.0, 321.0.

### Step 2. 2-(6-Bromo-2,3,4-trifluorophenyl)acetonitrile

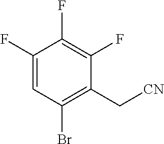 [SMILES: N#CCc1c(Br)cc(F)c(F)c1F]

To a solution of 6-bromo-2,3,4-trifluorobenzyl methanesulfonate (1.3 g, 4.1 mmol) in EtOH (11.3 mL)/water (2.3 mL) was added KCN (0.23 g, 4.48 mmol) and the mixture was heated at 70° C. for 1 h. After cooling to r.t., the mixture was concentrated, then diluted with DCM/water. The layers were separated and the organic layer was washed with sat. aq. NaHCO₃ and brine, dried over Na₂SO₄ and concentrated. The crude product was purified by FCC (0-20% EtOAc in hexanes) to provide the title compound (850 mg, 83%). LCMS calc. for C₃H₄BrF₃N (M+H)⁺ m/z=250.0, 252.0; found 250.0, 252.0.

### Step 3. Ethyl (4-bromo-3-cyano-6,7-difluorobenzo[b]thiophen-2-yl)carbamate

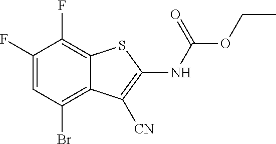 [SMILES: CCOC(=O)Nc1sc2c(F)c(F)cc(Br)c2c1C#N]

To a 0° C. solution of 2-(6-bromo-2,3,4-trifluorophenyl)acetonitrile (850 mg, 3.4 mmol) in DMF (8.5 mL) was added KOtBu (401 mg, 3.57 mmol). The mixture was stirred at 0° C. for 10 min., then ethoxy carbonyl isothiocyanate (412 μL, 3.57 mmol) was added and the mixture was warmed to r.t. After stirring for 1 h, the mixture was heated at 100° C. with stirring for 30 min. and was then allowed to cool to r.t. The reaction was quenched with water, and the resulting precipitate was collected by filtration, washed with cold water and hexanes, then air dried and used in the next step without further purification (901 mg, 73%). LCMS calc. for C₁₂H₃BrF₂N₂O₂S (M+H)⁺ m/z=361.0, 363.0; found 361.0, 363.0.

### Step 4. 2-Amino-4-bromo-6,7-difluorobenzo[b]thiophene-3-carbonitrile

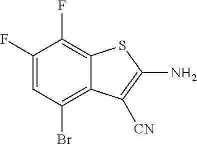 [SMILES: N#Cc1c(N)sc2c(F)c(F)cc(Br)c12]

To a solution of ethyl (4-bromo-3-cyano-6,7-difluorobenzo[b]thiophen-2-yl)carbamate (901 mg, 2.49 mmol) in DMSO (3.5 mL) was added NaOH (5.0 M, 2.7 mL, 13.7 mmol) and the mixture was stirred at 100° C. overnight. The mixture was cooled to r.t. and diluted with water. The resulting precipitate was collected by filtration, washed with water and hexanes, then dried and used in the next step without further purification (556 mg, 77%). LCMS calc. for C₉H₄BrF₂N₂S (M+H)⁺ m/z=289.0, 291.0; found 289.0, 291.0.

### Step 5. tert-Butyl (4-bromo-3-cyano-6,7-difluorobenzo[b]thiophen-2-yl)carbamate

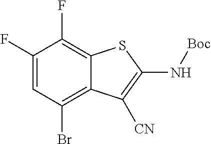 [SMILES: CC(C)(C)OC(=O)Nc1sc2c(F)c(F)cc(Br)c2c1C#N]

To a solution of 2-amino-4-bromo-6,7-difluorobenzo[b]thiophene-3-carbonitrile (556 mg, 1.92 mmol) in THF (10 mL) were added DMAP (9 mg, 0.08 mmol), DIPEA (400 μL, 2.31 mmol) and Boc₂O (420 mg, 1.92 mmol) and the mixture was stirred at r.t. overnight. The mixture was diluted with water and filtered. The filtrate was washed with water and air dried to provide the title compound, which was used in the next step without further purification. LCMS calc. for C₁₄H₁₂BrF₂N₂O₂S (M+H)⁺ m/z=389.0, 391.0; found 389.0, 391.0.

### Step 6. tert-Butyl (3-cyano-4-(5,5-dimethyl-1,3,2-dioxaborinan-2-yl)-6,7-difluorobenzo[b]thiophen-2-yl)carbamate

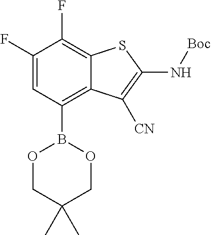 [SMILES: CC1(C)COB(c2cc(F)c(F)c3sc(NC(=O)OC(C)(C)C)c(C#N)c23)OC1]

To a mixture of tert-butyl (4-bromo-3-cyano-6,7-difluorobenzo[b]thiophen-2-yl)carbamate (200 mg, 0.51 mmol), 5,5,5′,5′-tetramethyl-2,2′-bi(1,3,2-dioxaborinane) (232 mg, 1.03 mmol), KOAc (151 mg, 1.54 mmol) and (dppf)PdCl₂ (42 mg, 0.05 mmol) was added 1,4-dioxane (5 mL) and the reaction flask was evacuated, back filled with N₂, then stirred at 100° C. for 2 h. The reaction mixture was then diluted with EtOAc and filtered. The filtrate was concentrated and the crude product purified by FCC (0-40% EtOAc in hexanes) to provide the title compound (145 mg, 67%). LCMS calc. for C₁₉H₂₂BF₂N₂O₄S (M+H)⁺ m/z=423.1; found 423.1.

### Step 7. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile

To a mixture of Intermediate 12 (50 mg, 0.10 mmol), tert-butyl (3-cyano-4-(5,5-dimethyl-1,3,2-dioxaborinan-2-yl)-6,7-difluorobenzo[b]thiophen-2-yl)carbamate (109 mg, 0.26 mmol), Pd-117 (15 mg, 0.021 mmol) and K₂CO₃ (43 mg, 0.31 mmol) was added 1,4-dioxane (1 mL) and the reaction flask was evacuated, back filled with N₂, then stirred at 90° C. overnight. The mixture was diluted with EtOAc and filtered through a plug of diatomaceous earth. The filtrate was washed with brine, dried over Na₂SO₄ and concentrated. The residue was purified by FCC (0-100% EtOAc in hexanes). The intermediate was dissolved in 1:1 DCM/TFA (1 mL) and stirred at r.t. for 30 min., then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 62a. Diastereomer 1. Peak 1. LCMS calc. for C₂₇H₁₆D₃ClF₆N₅OS (M+H)⁺ m/z=613.1; found 613.1. This is the potent peak.

Example 62b. Diastereomer 2. Peak 2. LCMS calc. for C₂₇H₁₆D₃ClF₆N₅OS (M+H)⁺ m/z=613.1; found 613.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; are prepared by an analogous procedure using the alternative diastereomer of Intermediate 12 as starting material.

Further isomers are prepared by using modifications to the procedure and employing appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile.

### Example 63a and 63b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile

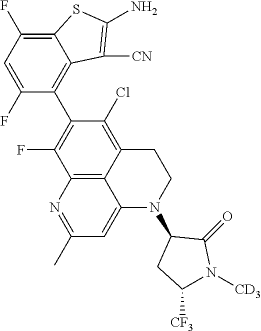 [SMILES: [2H]C([2H])([2H])N1C(=O)[C@H](N2CCc3c(Cl)c(-c4c(F)cc(F)c5sc(N)c(C#N)c45)c(F)c4nc(C)cc2c34)C[C@H]1C(F)(F)F]

The title compound was prepared according to the general procedures outlined for Example 62, Steps 2-7 utilizing 2-(2-bromo-3,5,6-trifluorophenyl)acetonitrile as the starting material in Step 2.

Example 63. LCMS calc. for C₂₇H₁₆D₃ClF₆N₅OS (M+H)⁺ m/z=613.1; found 613.1.

The compound is understood to comprise a mixture of atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; are prepared by an analogous procedure using the alternative diastereomer of Intermediate 12 as starting material.

Further isomers are prepared by using modifications to the procedure and employing appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile;

### Example 64a and 64b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile

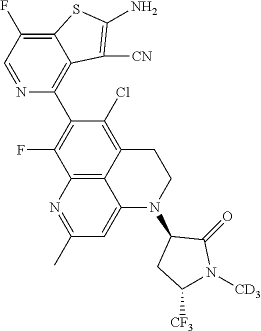 [SMILES: [2H]C([2H])([2H])N1C(=O)[C@H](N2CCc3c(Cl)c(-c4ncc(F)c5sc(N)c(C#N)c45)c(F)c4nc(C)cc2c34)C[C@H]1C(F)(F)F]

### Step 1. tert-Butyl (4-bromo-3-cyano-7-fluorothieno[3,2-c]pyridin-2-yl)carbamate

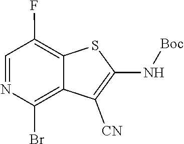 [SMILES: CC(C)(C)OC(=O)Nc1sc2c(F)cnc(Br)c2c1C#N]

To a 150 mL flask were added Boc₂O (9.64 g, 44.2 mmol), 2-amino-4-chloro-7-fluorothieno[3,2-c]pyridine-3-carbonitrile (5.03 g, 22.10 mmol), DMAP (0.054 g, 0.442 mmol), DIPEA (4.6 mL, 26.5 mmol) and THF (31 mL) under N₂. The resulting mixture was stirred at 70° C. for 4 h. The mixture was concentrated, then triturated with 1:1 hexanes/EtOAc (100 mL). The filtrate was concentrated and further purified by FCC (0-5% EtOAc in DCM) to provide the title compound (5.9 g, 81%). LCMS calc. for C₁₃H₁₂BrFN₃O₂S (M+H)⁺ m/z=372.0, 374.0; found 372.0, 374.0.

### Step 2. tert-Butyl (4-bromo-3-cyano-7-fluorothieno[3,2-c]pyridin-2-yl)((2-(trimethylsilyl)ethoxy)methyl)carbamate

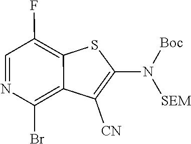 [SMILES: CC(C)(C)OC(=O)N(COCC[Si](C)(C)C)c1sc2c(F)cnc(Br)c2c1C#N]

To a vial containing tert-butyl (4-chloro-3-cyano-7-fluorothieno[3,2-c]pyridin-2-yl)carbamate (2.04 g, 6.21 mmol) in DMF (12 mL) at 0° C. was added NaH (60% w/w in mineral oil, 0.27 g, 6.83 mmol) and the mixture was stirred at r.t. for 30 min. The reaction vial was cooled to 0° C. SEMCl (1.47 mL, 7.46 mmol) was added and the reaction mixture was stirred at r.t. for 1 h. The reaction was quenched with aq.NaHCO₃ and extracted with EtOAc. The organic layer was washed with water and brine, dried over Na₂SO₄ and concentrated. The crude product was purified by FCC (0-20% EtOAc in hexane) to provide the title compound (2.46 g, 87%). LCMS calc. for C₁₉H₂₆BrFN₃O₃SSi (M+H)⁺ m/z=502.0, 504.0; found 502.0, 504.0.

### Step 3. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile

To a solution of Intermediate 12 (81 mg, 0.167 mmol) in THF (1.0 mL) at −78° C. was added iPrMgCl·LiCl (1.3 M in THF, 0.265 mL, 0.344 mmol), the mixture was stirred at −78° C. for 45 min. Then ZnCl₂ (1.9 M in 2-MeTHF, 0.69 mL, 0.34 mmol) was added dropwise and the mixture was warmed to r.t. The mixture was then transferred into a vial containing Ruphos-G4 (26 mg, 0.033 mmol), tert-butyl (4-chloro-3-cyano-7-fluorothieno[3,2-c]pyridin-2-yl)((2-(trimethylsilyl)ethoxy)methyl)carbamate (75 mg, 0.164 mmol) and THF (0.8 mL). The mixture was heated at 60° C. for 2 h, then cooled to r.t. The reaction was quenched with sat. aq. NaHCO₃ and extracted with EtOAc. The organic layer was washed with water and brine, dried over Na₂SO₄ and concentrated. The crude product was purified by FCC (0-10% MeOH in DCM). The intermediate was dissolved in 1:1 DCM/TFA (1 mL) and the mixture was stirred at r.t. for 30 min., then concentrated. The crude product was diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 64a. Diastereomer 1. Peak 1. LCMS calc. for C₂₆H₁₆D₃ClF₅N₆OS (M+H)⁺ m/z=596.1; found 596.1. This is the potent peak.

Example 64b. Diastereomer 2. Peak 2. LCMS calc. for C₂₆H₁₆D₃ClF₅N₆OS (M+H)⁺ m/z=596.1; found 596.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; are prepared by an analogous procedure using the alternative diastereomer of Intermediate 12 as starting material.

Further isomers are prepared by using modifications to the procedure and employing appropriate starting materials, i.e., (Rₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; (Sₐ)-2-amino-4-(7-chloro-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; (Rₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile; and (Sₐ)-2-amino-4-(7-chloro-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile.

### Example 65a and 65b. 2-Amino-7-fluoro-4-(9-fluoro-2-methyl-4-((3R,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile

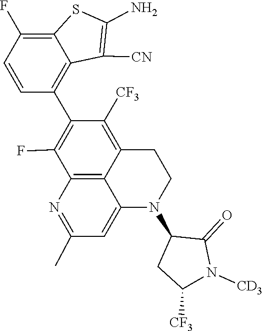 [SMILES: [2H]C([2H])([2H])N1C(=O)[C@H](N2CCc3c(C(F)(F)F)c(-c4ccc(F)c5sc(N)c(C#N)c45)c(F)c4nc(C)cc2c34)C[C@H]1C(F)(F)F]

### Step 1. (3R,5S)-3-(8-Bromo-9-fluoro-7-iodo-2-methyl-5,6-dihydro-4H-benzo[de][1,6]naphthyridin-4-yl)-1-(methyl-d₃)-5-(trifluoromethyl)pyrrolidin-2-one

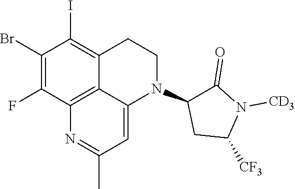 [SMILES: [2H]C([2H])([2H])N1C(=O)[C@H](N2CCc3c(I)c(Br)c(F)c4nc(C)cc2c34)C[C@H]1C(F)(F)F]

To the mixture of Intermediate 7 (237 mg, 0.54 mmol) and Intermediate 11 (199 mg, 1.07 mmol), in THF (2.3 mL), DCM (2.3 mL) and HFIP (767 μL) were added NaBH(OAc)₃ (455 mg, 2.15 mmol) and AcOH (331 μL, 4.29 mmol) at r.t., the resulting mixture was stirred at r.t. overnight. The reaction was quenched with aq. NaHCO₃ and diluted with EtOAc. The layers were separated and the organic layer was washed with water and brine, dried over Na₂SO₄ and concentrated. The crude product was purified by FCC (0-100% EtOAc in hexane) to provide the title compound (263 mg, 49%). LCMS calc. for C₁₃H₁₃D₃BrF₄IN₃O (M+H)⁺ m/z=575.0, 577.0; found 575.0, 577.0.

### Step 2. (3R,5S)-3-(8-Bromo-9-fluoro-2-methyl-7-(trifluoromethyl)-5,6-dihydro-4H-benzo[de][1,6]naphthyridin-4-yl)-1-(methyl-d₃)-5-(trifluoromethyl)pyrrolidin-2-one

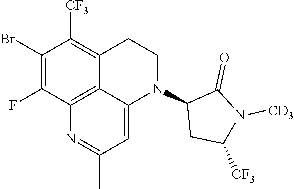 [SMILES: [2H]C([2H])([2H])N1C(=O)[C@H](N2CCc3c(C(F)(F)F)c(Br)c(F)c4nc(C)cc2c34)C[C@H]1C(F)(F)F]

To a 8 mL vial charged with stir a bar were added (3R,5S)-3-(8-bromo-9-fluoro-7-iodo-2-methyl-5,6-dihydro-4H-benzo[de][1,6]naphthyridin-4-yl)-1-(methyl-d₃)-5-(trifluoromethyl)pyrrolidin-2-one (103 mg, 0.18 mmol), CuI (10.2 mg, 0.054 mmol), NMP (895 μL), and methyl fluorosulfonyldifluoroacetate (56.5 μL, 0.447 mmol) under N₂ at r.t. The resulting mixture was warmed to 80° C. for 6 h. The mixture was cooled to r.t., diluted with EtOAc and washed with sat. aq. NaHCO₃ and brine, dried over Na₂SO₄ and concentrated. The crude product was purified by FCC (0-100% EtOAc in hexanes) to provide the title compound (54 mg, 58%). LCMS calc. for C₁₉H₁₃D₃BrF₇N₃O (M+H)⁺ m/z=517.0, 519.0; found 517.0, 519.0.

### Step 3. 2-Amino-7-fluoro-4-(9-fluoro-2-methyl-4-((3R,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile

To a mixture of dichloro[bis(2-(diphenylphosphino)phenyl)ether]palladium(II) (9 mg, 0.013 mmol), Cs₂CO₃ (51 mg, 0.156 mmol), tert-butyl (3-cyano-4-(5,5-dimethyl-1,3,2-dioxaborinan-2-yl)-7-fluorobenzo[b]thiophen-2-yl)carbamate (42 mg, 0.104 mmol), and (3R,5S)-3-(8-bromo-9-fluoro-2-methyl-7-(trifluoromethyl)-5,6-dihydro-4H-benzo[de][1,6]naphthyridin-4-yl)-1-(methyl-d₃)-5-(trifluoromethyl)pyrrolidin-2-one (27 mg, 0.052 mmol) was added 1,4-dioxane (520 μL) under N₂. The mixture was heated at 100° C. for 2 h, then cooled to r.t., diluted with EtOAc and filtered. The filtrate was concentrated and the residue was stirred in 1:1 DCM/TFA (1 mL) at r.t. for 30 min. The mixture was concentrated, the residue dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 65a. Diastereomer 1. Peak 1. LCMS calc. for C₂₈H₁₇D₃F₈N₅OS (M+H)⁺ m/z=629.1; found 629.1. This is the potent peak.

Example 65b. Diastereomer 2. Peak 2. LCMS calc. for C₂₈H₁₇D₃F₈N₅OS (M+H)⁺ m/z=629.1; found 629.1.

The compounds are understood to comprise atropisomers, i.e., (Rₐ)-2-amino-7-fluoro-4-(9-fluoro-2-methyl-4-((3R,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; and (Sₐ)-2-amino-7-fluoro-4-(9-fluoro-2-methyl-4-((3R,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile.

The alternative isomers, i.e., (Rₐ)-2-amino-7-fluoro-4-(9-fluoro-2-methyl-4-((3S,5R)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; and (Sₐ)-2-amino-7-fluoro-4-(9-fluoro-2-methyl-4-((3S,5R)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; are prepared by an analogous procedure using the alternative diastereomer of Intermediate 11 as starting material.

Further isomers are prepared by using modifications to the procedure and employing appropriate starting materials, i.e., (Rₐ)-2-amino-7-fluoro-4-(9-fluoro-2-methyl-4-((3R,5R)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; (Sₐ)-2-amino-7-fluoro-4-(9-fluoro-2-methyl-4-((3R,5R)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; (Rₐ)-2-amino-7-fluoro-4-(9-fluoro-2-methyl-4-((3S,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile; and (Sₐ)-2-amino-7-fluoro-4-(9-fluoro-2-methyl-4-((3S,5S)-1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile.

### Example 66a and 66b. 2-Amino-4-(7-(difluoromethyl)-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

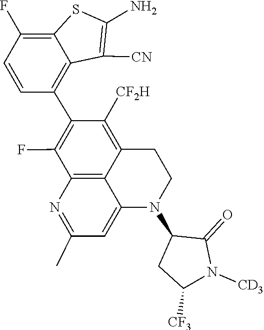 [SMILES: [2H]C([2H])([2H])N1C(=O)[C@H](N2CCc3c(C(F)F)c(-c4ccc(F)c5sc(N)c(C#N)c45)c(F)c4nc(C)cc2c34)C[C@H]1C(F)(F)F]

### Step 1. (3R,5S)-3-(8-Bromo-7-(difluoromethyl)-9-fluoro-2-methyl-5,6-dihydro-4H-benzo[de][1,6]naphthyridin-4-yl)-1-(methyl-d₃)-5-(trifluoromethyl)pyrrolidin-2-one

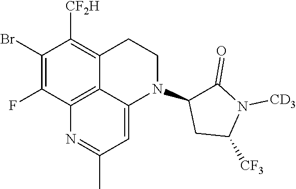 [SMILES: [2H]C([2H])([2H])N1C(=O)[C@H](N2CCc3c(C(F)F)c(Br)c(F)c4nc(C)cc2c34)C[C@H]1C(F)(F)F]

To a 8 mL vial charged with a stir bar were added KOtBu (45 mg, 0.40 mmol) followed by DMF (700 μL). The mixture was stirred at r.t. for 5 min. then a DMF (700 μL) solution of 1,10-phenanthroline (36 mg, 0.20 mmol) was added to the mixture and stirring was continued at r.t. for an additional 10 min. In a separate vial were added (3R,5S)-3-(8-bromo-9-fluoro-7-iodo-2-methyl-5,6-dihydro-4H-benzo[de][1,6]naphthyridin-4-yl)-1-(methyl-d₃)-5-(trifluoromethyl) pyrrolidin-2-one (Example 65, Step 1, 76 mg, 0.133 mmol), (difluoromethyl)trimethyl silane (56 μL, 0.40 mmol) and DMF (700 μL). The reaction flask was placed under a N₂ atmosphere, then the contents of the first vial were added and the mixture was stirred at r.t. for 5 h. The reaction was quenched with water and extracted with EtOAc. The organic layer was washed with water and brine, dried over Na₂SO₄ and concentrated. The crude product was purified by FCC (0-100% EtOAc in hexanes) to provide the title compound (24 mg, 36%). LCMS calc. for C₁₉H₁₄D₃BrF₆N₃O (M+H)⁺ m/z=499.0, 501.0; found 499.0, 501.0.

### Step 2. 2-Amino-4-(7-(difluoromethyl)-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of dichloro[bis(2-(diphenylphosphino)phenyl)ether]palladium(II) (8 mg, 0.01 mmol), Cs₂CO₃ (31 mg, 0.095 mmol), tert-butyl (3-cyano-4-(5,5-dimethyl-1,3,2-dioxaborinan-2-yl)-7-fluorobenzo[b]thiophen-2-yl)carbamate (39 mg, 0.095 mmol), and (3R,5S)-3-(8-bromo-7-(difluoromethyl)-9-fluoro-2-methyl-5,6-dihydro-4H-benzo[de][1,6]naphthyridin-4-yl)-1-(methyl-d₃)-5-(trifluoromethyl)pyrrolidin-2-one (24 mg, 0.048 mmol) was added 1,4-dioxane (500 μL) under N₂. The mixture was heated at 100° C. for 2 h. The mixture was cooled to r.t., diluted with EtOAc and filtered. The filtrate was concentrated and the residue was dissolved in 1:1 DCM/TFA (1 mL) and the mixture was stirred for 30 min. at r.t. The mixture was concentrated and the residue was dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 66a. Diastereomer 1. Peak 1. LCMS calc. for C₂₈H₁₈D₃F₇N₅OS (M+H)⁺ m/z=611.1; found 611.1. This is the potent peak.

Example 66b. Diastereomer 2. Peak 2. LCMS calc. for C₂₈H₁₈D₃F₇N₅OS (M+H)⁺ m/z=611.1; found 611.1.

The compounds are understood to comprise atropisomers, i.e., (Rₐ)-2-amino-4-(7-(difluoromethyl)-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-(difluoromethyl)-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-(difluoromethyl)-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-(difluoromethyl)-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using (3S,5R)-3-(8-bromo-9-fluoro-7-iodo-2-methyl-5,6-dihydro-4H-benzo[de][1,6]naphthyridin-4-yl)-1-(methyl-d₃)-5-(trifluoromethyl) pyrrolidin-2-one (diastereomer of Example 61, Step 1) as starting material.

Further isomers are prepared by using modifications to the procedure and employing appropriate starting materials, i.e., (Rₐ)-2-amino-4-(7-(difluoromethyl)-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-(difluoromethyl)-9-fluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-(difluoromethyl)-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-(difluoromethyl)-9-fluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

Example 67a and 67b. 2-Amino-4-(7-chloro-3,9-difluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

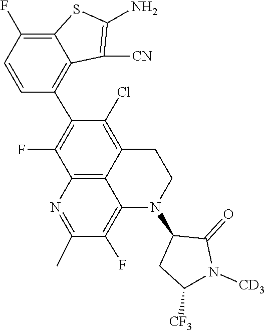 [SMILES: [2H]C([2H])([2H])N1C(=O)[C@H](N2CCc3c(Cl)c(-c4ccc(F)c5sc(N)c(C#N)c45)c(F)c4nc(C)c(F)c2c34)C[C@H]1C(F)(F)F]

In a 4 mL vial were added NFSI (26 mg, 0.084 mmol) and tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (Example 41, 23 mg, 0.034 mmol). The reaction flask was evacuated and back filled with N₂, then MeCN (500 μL) and HFIP (20 μL) were added, the resulting mixture was heated at 95° C. for 30 min. The mixture was then concentrated and purified by FCC (0-10% MeOH in DCM). The intermediate was dissolved in 1:1 DCM/TFA (1 mL) and the mixture was stirred at r.t. for 20 min., then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 67a. Diastereomer 1. Peak 1. LCMS calc. for C₂₇H₁₆D₃ClF₆N₅OS (M+H)⁺ m/z=613.1; found 613.1. This is the potent peak.

Example 67b. Diastereomer 2. Peak 2. LCMS calc. for C₂₇H₁₆D₃ClF₆N₅OS (M+H)⁺ m/z=613.1; found 613.1.

The compounds are understood to comprise atropisomers, i.e., (Rₐ)-2-amino-4-(7-chloro-3,9-difluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-3,9-difluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-3,9-difluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-3,9-difluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using tert-butyl (4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (diastereomer of Example 41) as starting material.

Further isomers are prepared by using modifications to the procedure and employing appropriate starting materials, i.e., (Rₐ)-2-amino-4-(7-chloro-3,9-difluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-3,9-difluoro-2-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-3,9-difluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-3,9-difluoro-2-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 68a and 68b. 2-Amino-7-fluoro-4-(9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile

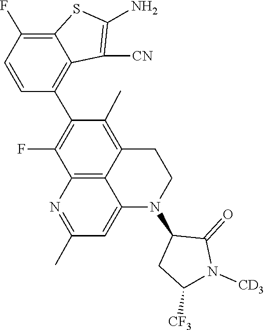 [SMILES: [2H]C([2H])([2H])N1C(=O)[C@H](N2CCc3c(C)c(-c4ccc(F)c5sc(N)c(C#N)c45)c(F)c4nc(C)cc2c34)C[C@H]1C(F)(F)F]

In a 4 mL vial were combined K₃PO₄ (62 mg, 0.29 mmol), SPhos Pd G4 (17 mg, 0.022 mmol), methylboronic acid (43 mg, 0.725 mmol), and tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (Example 41, 50 mg, 0.073 mmol). The reaction vessel was evacuated and back filled with N₂, then 1,4-dioxane (300 μL)/water (60 μL) was added and the resulting mixture was heated at 95° C. for 8 h. After cooling to r.t. the reaction was quenched with sat. aq. NaHCO₃ and extracted with EtOAc. The organic layer was washed with brine, dried over Na₂SO₄ and concentrated. The crude product was purified by FCC (0-10% MeOH in DCM). The intermediate was dissolved in 1:1 DCM/TFA (1 mL) and stirred at r.t. for 30 min., then concentrated, redissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 68a. Diastereomer 1. Peak 1. LCMS calc. for C₂₈H₂₀D₃F₅N₅OS (M+H)⁺ m/z=575.2; found 575.2. This is the potent peak.

Example 68b. Diastereomer 2. Peak 2. LCMS calc. for C₂₈H₂₀D₃F₅N₅OS (M+H)⁺ m/z=575.2; found 575.2.

The compounds are understood to comprise atropisomers, i.e., (Rₐ)-2-amino-7-fluoro-4-(9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-7-fluoro-4-(9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile.

The alternative isomers, i.e., (Rₐ)-2-amino-7-fluoro-4-(9-fluoro-2,7-dimethyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-7-fluoro-4-(9-fluoro-2,7-dimethyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; are prepared by an analogous procedure using tert-butyl (4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (diastereomer of Example 41) as starting material.

Further isomers are prepared by using modifications to the procedure and employing appropriate starting materials, i.e., (Rₐ)-2-amino-7-fluoro-4-(9-fluoro-2,7-dimethyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; (Sₐ)-2-amino-7-fluoro-4-(9-fluoro-2,7-dimethyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; (Rₐ)-2-amino-7-fluoro-4-(9-fluoro-2,7-dimethyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile; and (Sₐ)-2-amino-7-fluoro-4-(9-fluoro-2,7-dimethyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile.

### Example 69a and 69b. 2-Amino-4-(2-(difluoromethyl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

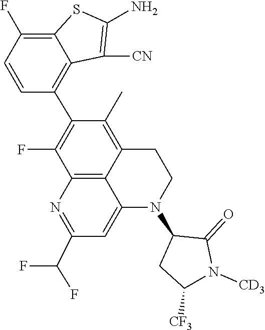 [SMILES: [2H]C([2H])([2H])N1C(=O)[C@H](N2CCc3c(C)c(-c4ccc(F)c5sc(N)c(C#N)c45)c(F)c4nc(C(F)F)cc2c34)C[C@H]1C(F)(F)F]

### Step 1. 7-Bromo-6-chloro-2-(difluoromethyl)-5-(2,2-dimethoxyethyl)-8-fluoro-4-(methylthio)quinoline

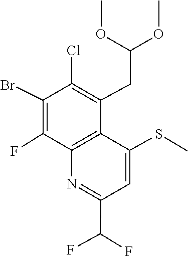 [SMILES: COC(Cc1c(Cl)c(Br)c(F)c2nc(C(F)F)cc(SC)c12)OC]

To a 8 mL via was added KOtBu (447 mg, 4.0 mmol) followed by DMF (4.2 mL) under N₂, the mixture was stir at r.t. for 5 min. before (difluoromethyl)trimethylsilane (564 μL, 3.98 mmol) was added. The mixture was stirred for an additional 10 min., then a DMF (4.2 mL) solution of 1,10-phenanthroline (359 mg, 1.992 mmol) was added and stirring was continued for another 10 min. The contents were then transferred to a separate vial containing 7-bromo-6-chloro-5-(2,2-dimethoxyethyl)-8-fluoro-2-iodo-4-(methylthio)quinoline (Intermediate 9, Step 1, 864 mg, 1.660 mmol a syringe, the resulting mixture was stirred at r.t. for 3 h. The mixture was diluted with EtOAc and sat. aq. NaHCO₃, then filtered through diatomaceous earth. The filtrate was phase separated and the organic layer was washed with brine, dried over Na₂SO₄ and concentrated. The crude product was purified by FCC (0-60% EtOAc in hexanes) to provide the title compound (511 mg, 69%). LCMS calc. for C₁₅H₁₅BrClF₃NO₂S (M+H)⁺ m/z=444.0, 446.0; found 444.0, 446.0.

### Step 2. 2-Amino-4-(6-chloro-2-(difluoromethyl)-5-(2,2-dimethoxyethyl)-8-fluoro-4-(methylthio)quinolin-7-yl)-7-fluorobenzo[b]thiophene-3-carbonitrile

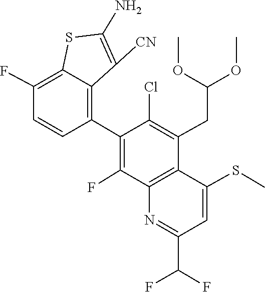 [SMILES: COC(Cc1c(Cl)c(-c2ccc(F)c3sc(N)c(C#N)c23)c(F)c2nc(C(F)F)cc(SC)c12)OC]

To the mixture of dichloro[bis(2-(diphenylphosphino)phenyl)ether]palladium(II) (206 mg, 0.287 mmol), Cs₂CO₃ (1.9 g, 5.75 mmol), tert-butyl (3-cyano-4-(5,5-dimethyl-1,3,2-dioxaborinan-2-yl)-7-fluorobenzo[b]thiophen-2-yl)carbamate (929 mg, 2.3 mmol), and 7-bromo-6-chloro-2-(difluoromethyl)-5-(2,2-dimethoxyethyl)-8-fluoro-4-(methylthio)quinoline (511 mg, 1.15 mmol) was added 1,4-dioxane (11.5 mL) under N₂. The mixture was heated at 100° C. for 2 h, then diluted with EtOAc and filtered through a plug of diatomaceous earth. The filtrate was concentrated and the residue was purified by FCC (0-100% EtOAc in hexanes) to provide the title compound (539 mg, 71%). LCMS calc. for C₂₄H₁₉ClF₄N₃O₂S₂ (M+H)⁺ m/z=556.1; found 556.1.

### Step 3. 2-Amino-4-(2-(difluoromethyl)-5-(2,2-dimethoxyethyl)-8-fluoro-6-methyl-4-(methylthio)quinolin-7-yl)-7-fluorobenzo[b]thiophene-3-carbonitrile

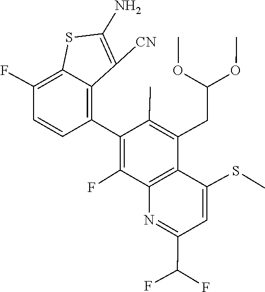 [SMILES: COC(Cc1c(C)c(-c2ccc(F)c3sc(N)c(C#N)c23)c(F)c2nc(C(F)F)cc(SC)c12)OC]

In a 4 mL vial were added K3PO₄ (121 mg, 0.572 mmol), SPhos Pd G4 (54 mg, 0.069 mmol), methylboronic acid (137 mg, 2.29 mmol), and tert-butyl (4-(6-chloro-2-(difluoromethyl)-5-(2,2-dimethoxyethyl)-8-fluoro-4-(methylthio)quinolin-7-yl)-3-cyano-7-fluorobenzo[b]thiophen-2-yl)carbamate (150 mg, 0.229 mmol). The reaction flask was evacuated and back filled with N₂, then 1,4-dioxane (1.0 mL) and water (200 μL) were added. The mixture was heated at 95° C. for 2 h. The reaction was quenched with sat. aq. NaHCO₃ and extracted with EtOAc. The organic layer was washed with brine, dried over Na₂SO₄ and concentrated. The crude product was purified by FCC (0-100% EtOAc in hexanes) to provide the title compound (88 mg, 60%). LCMS calc. for C₂₅H₂₂F₄N₃O₂S₂ (M+H)⁺ m/z=536.1; found 536.1.

### Step 4. 2-Amino-4-(2-(difluoromethyl)-8-fluoro-6-methyl-4-(methylthio)-5-(2-oxoethyl)quinolin-7-yl)-7-fluorobenzo[b]thiophene-3-carbonitrile

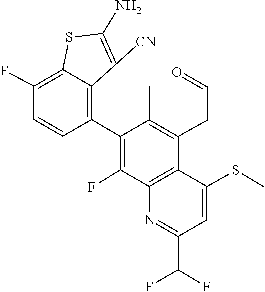 [SMILES: CSc1cc(C(F)F)nc2c(F)c(-c3ccc(F)c4sc(N)c(C#N)c34)c(C)c(CC=O)c12]

To a solution of tert-butyl (3-cyano-4-(2-(difluoromethyl)-5-(2,2-dimethoxyethyl)-8-fluoro-6-methyl-4-(methylthio)quinolin-7-yl)-7-fluorobenzo[b]thiophen-2-yl)carbamate (88 mg, 0.14 mmol) in DCM/acetone (4:1, 2.2 mL total volume) was added iron (Ill) chloride hexahydrate (82 mg, 0.30 mmol) and the mixture was stirred at r.t. for 30 min. The reaction was quenched with sat. aq. NaHCO₃ and extracted with EtOAc. The organic layer was washed with brine, dried over Na₂SO₄ and concentrated. The crude product was used in the next step without further purification. LCMS calc. for C₂₃H₁₆F₄N₃OS₂ (M+H)⁺ m/z=490.1; found 490.1.

### Step 5. 2-Amino-4-(2-(difluoromethyl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To a mixture of tert-butyl (3-cyano-4-(2-(difluoromethyl)-8-fluoro-6-methyl-4-(methylthio)-5-(2-oxoethyl)quinolin-7-yl)-7-fluorobenzo[b]thiophen-2-yl)carbamate (81 mg, 0.138 mmol) and Intermediate 11 (64 mg, 0.345 mmol), in THF (591 μL), DCM (591 μL) and HFIP (197 μL) were added NaBH(OAc)₃ (117 mg, 0.552 mmol) and AcOH (85 μL, 1.104 mmol) at r.t., the resulting mixture was stirred at r.t. overnight. The reaction was quenched with sat. aq. NaHCO₃ and extracted with EtOAc. The organic layer was washed with brine, dried over Na₂SO₄ and concentrated. The crude product was purified by FCC (0-100% EtOAc in hexanes). The intermediate was dissolved in 1:1 DCM/TFA (1 mL) and the mixture was stirred at r.t. for 20 min., then concentrated. The residue was dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 69a. Diastereomer 1. Peak 1. LCMS calc. for C₂₈H₁₈D₃F₇N₆OS (M+H)⁺ m/z=611.1; found 611.1. This is the potent peak. ¹H NMR (500 MHz, MeOD) δ 7.21 (dd, J=8.3, 5.1 Hz, 1H), 7.10-7.03 (m, 1H), 6.95 (s, 1H), 6.72 (t, J=55.9 Hz, 1H), 5.21 (t, J=9.4 Hz, 1H), 4.50 (p, J=7.6 Hz, 1H), 3.54 (ddd, J=11.6, 8.9, 5.5 Hz, 1H), 3.43 (dt, J=11.4, 5.4 Hz, 1H), 3.22 (q, J=5.3 Hz, 2H), 2.81-2.72 (m, 1H), 2.59 (dt, J=14.4, 9.6 Hz, 1H), 2.12 (s, 3H).

Example 69b. Diastereomer 2. Peak 2. LCMS calc. for C₂₈H₁₈D₃F₇N₅OS (M+H)⁺ m/z=611.1; found 611.1.

The compounds are understood to comprise atropisomers, i.e., (Rₐ)-2-amino-4-(2-(difluoromethyl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(difluoromethyl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(2-(difluoromethyl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(difluoromethyl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

Further isomers are prepared by using modifications to the procedure and employing appropriate starting materials, i.e., (Rₐ)-2-amino-4-(2-(difluoromethyl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(2-(difluoromethyl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(2-(difluoromethyl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(difluoromethyl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 70a and 70b. 2-Amino-4-(2-(3-(dimethylamino)-3-methylazetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

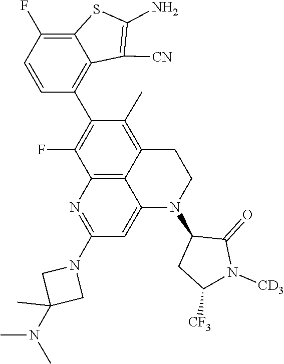 [SMILES: [2H]C([2H])([2H])N1C(=O)[C@H](N2CCc3c(C)c(-c4ccc(F)c5sc(N)c(C#N)c45)c(F)c4nc(N5CC(C)(N(C)C)C5)cc2c34)C[C@H]1C(F)(F)F]

The title compound was prepared according to the procedure for Example 64, utilizing Example 49 as the starting material. The compound was prepared as two diastereomers, which were separated during prep. HPLC purification.

Example 70a. Diastereomer 1. Peak 1. LCMS calc. for C₃₃H₃₀D₃F₅N₇OS (M+H)⁺ m/z=673.2; found 673.2. This is the potent peak.

Example 70b. Diastereomer 2. Peak 2. LCMS calc. for C₃₃H₃₀D₃F₅N₇OS (M+H)⁺ m/z=673.2; found 673.2.

The compounds are understood to comprise atropisomers, i.e., (Rₐ)-2-amino-4-(2-(3-(dimethylamino)-3-methylazetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)-3-methylazetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(2-(3-(dimethylamino)-3-methylazetidin-1-yl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)-3-methylazetidin-1-yl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Example 49 as starting material.

Further isomers are prepared by using modifications to the procedure and employing appropriate starting materials, i.e., (Rₐ)-2-amino-4-(2-(3-(dimethylamino)-3-methylazetidin-1-yl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)-3-methylazetidin-1-yl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(2-(3-(dimethylamino)-3-methylazetidin-1-yl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)-3-methylazetidin-1-yl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 71a and 71b. 2-Amino-4-(2-(3-(dimethylamino)azetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

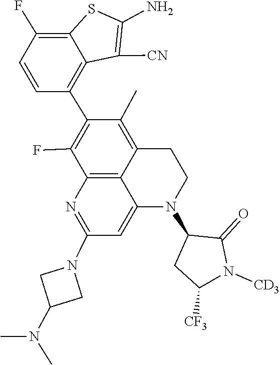 [SMILES: [2H]C([2H])([2H])N1C(=O)[C@H](N2CCc3c(C)c(-c4ccc(F)c5sc(N)c(C#N)c45)c(F)c4nc(N5CC(N(C)C)C5)cc2c34)C[C@H]1C(F)(F)F]

The title compound was prepared according to the procedure outline for Example 66, utilizing Example 48 as the starting material. The compound was prepared as a mixture of diastereomers which were separated during prep. HPLC purification.

Example 71a. Diastereomer 1. Peak 1. LCMS calc. for C₃₂H₂₈D₃F₅N₇OS (M+H)⁺ m/z=659.2; found 659.2. This is the potent peak.

Example 71b. Diastereomer 2. Peak 2. LCMS calc. for C₃₂H₂₈D₃F₅N₇OS (M+H)⁺ m/z=659.2; found 659.2.

The compounds are understood to comprise atropisomers, i.e., (Rₐ)-2-amino-4-(2-(3-(dimethylamino)azetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)azetidin-1-yl)-9-fluoro-7-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(2-(3-(dimethylamino)azetidin-1-yl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)azetidin-1-yl)-9-fluoro-7-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Example 48 as starting material.

Further isomers are prepared by using modifications to the procedure and employing appropriate starting materials, i.e., (Rₐ)-2-amino-4-(2-(3-(dimethylamino)azetidin-1-yl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(2-(3-(dimethylamino)azetidin-1-yl)-9-fluoro-7-methyl-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(2-(3-(dimethylamino)azetidin-1-yl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(3-(dimethylamino)azetidin-1-yl)-9-fluoro-7-methyl-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 72a and 72b. 2-Amino-4-(7-chloro-9-fluoro-4-((3S,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile

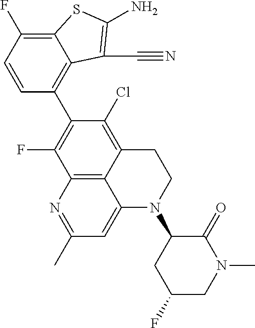 [SMILES: Cc1cc2c3c(c(Cl)c(-c4ccc(F)c5sc(N)c(C#N)c45)c(F)c3n1)CCN2[C@@H]1C[C@@H](F)CN(C)C1=O]

### Step 1. (3R,5R)-3-(8-Bromo-7-chloro-9-fluoro-2-methyl-5,6-dihydro-4H-benzo[de][1,6]naphthyridin-4-yl)-5-fluoropiperidin-2-one

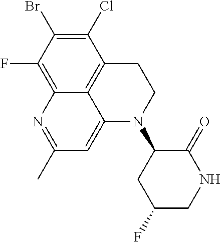 [SMILES: Cc1cc2c3c(c(Cl)c(Br)c(F)c3n1)CCN2[C@@H]1C[C@@H](F)CNC1=O]

To a mixture of Intermediate 8 (125 mg, 0.345 mmol) and (3R,5R)-3-amino-5-fluoropiperidin-2-one (68 mg, 0.52 mmol) in THF/MeOH/HFIP (3:3:1, 6.8 mL total volume) was added NaBH₃CN (125 mg, 0.345 mmol) and the mixture was stirred at r.t. for 30 min. The mixture was then concentrated, diluted with MeCN/MeOH/DMSO (2:2:1, 10 mL total volume), then filtered. The filtrate was purified by prep. HPLC (XBRIDGE® C₁₈ column, eluting with a gradient of MeCN/water containing 0.1% NH₄OH, at flow rate of 60 mL/min.) to afford the title compound. LCMS calc. for C₁₇H₁₆BrClF₂N₃O (M+H)⁺ m/z=430.0, 432.0; found 430.0, 432.0.

### Step 2. (3R,5R)-3-(8-Bromo-7-chloro-9-fluoro-2-methyl-5,6-dihydro-4H-benzo[de][1,6]naphthyridin-4-yl)-5-fluoro-1-methylpiperidin-2-one

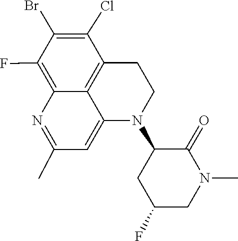 [SMILES: Cc1cc2c3c(c(Cl)c(Br)c(F)c3n1)CCN2[C@@H]1C[C@@H](F)CN(C)C1=O]

To a solution of (3R,5R)-3-(8-bromo-7-chloro-9-fluoro-2-methyl-5,6-dihydro-4H-benzo[de][1,6]naphthyridin-4-yl)-5-fluoropiperidin-2-one (80 mg, 0.19 mmol) in THF (1.8 mL) at 0° C. was added NaH (60% w/w in mineral oil, 15 mg, 0.37 mmol) and the mixture was stirred at 0° C. for 10 min., then Mel (23 μL, 0.37 mmol) was added and stirring was continued at r.t. for 30 min. The reaction was quenched with sat. ammonium chloride and extracted with EtOAc. The organic layer was washed with water and brine, dried over Na₂SO₄ and concentrated. The crude product was used in the next step without further purification. LCMS calc. for C₁₃H₁₃BrClF₂N₃O (M+H)⁺ m/z=444.0, 446.0; found 444.0, 446.0.

### Step 3. 2-Amino-4-(7-chloro-9-fluoro-4-((3S,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile

To a mixture of (3R,5R)-3-(8-bromo-7-chloro-9-fluoro-2-methyl-5,6-dihydro-4H-benzo[de][1,6]naphthyridin-4-yl)-5-fluoro-1-methylpiperidin-2-one (50 mg, 0.11 mmol), tert-butyl (3-cyano-4-(5,5-dimethyl-1,3,2-dioxaborinan-2-yl)-7-fluorobenzo[b]thiophen-2-yl)carbamate (91 mg, 0.22 mmol), dichloro[bis(diphenylphosphinophenyl)ether]palladium(II) (20 mg, 0.03 mmol) and Cs₂CO₃ (183 mg, 0.56 mmol) was added 1,4-dioxane (750 μL) and the reaction flask was evacuated, back filled with N₂, then stirred at 100° C. for 1 h. The mixture was concentrated, diluted with MeOH, filtered, then reconcentrated. The residue was dissolved in 1:1 TFA/MeCN (1 mL) and stirred at 60° C. for 1 h, then purified by prep. HPLC (XBRIDGE® C₁₈ column, eluting with a gradient of MeCN/water containing 0.1% TFA, at flow rate of 60 mL/min.) to afford the title compound as two peaks.

Example 72a. Diastereomer 1. Peak 1. LCMS calc. for C₂₇H₂₂ClF₃N₅OS (M+H)⁺ m/z=556.1; found 556.1. This is the potent peak.

Example 72b. Diastereomer 2. Peak 2. LCMS calc. for C₂₇H₂₂ClF₃N₅OS (M+H)⁺ m/z=556.1; found 556.1.

The compounds are understood to comprise atropisomers, i.e., (Rₐ)-2-amino-4-(7-chloro-9-fluoro-4-((3S,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3R,5S)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; are prepared by an analogous procedure using the alternative diastereomer of the reactant of step 1 as starting material.

Further isomers are prepared by using modifications to the procedure and employing appropriate starting materials, i.e., (Rₐ)-2-amino-4-(7-chloro-9-fluoro-4-((3S,5S)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5S)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5R)-5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile.

### Example 73. 2-Amino-4-(7-chloro-9-fluoro-2-(3-hydroxy-3-methylbut-1-yn-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

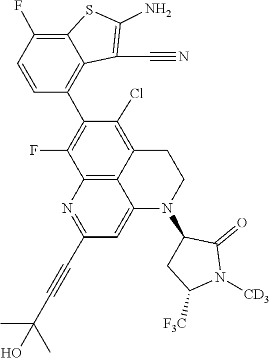 [SMILES: [2H]C([2H])([2H])N1C(=O)[C@H](N2CCc3c(Cl)c(-c4ccc(F)c5sc(N)c(C#N)c45)c(F)c4nc(C#CC(C)(C)O)cc2c34)C[C@H]1C(F)(F)F]

### Step 1. (3R,5S)-3-(8-Bromo-7-chloro-9-fluoro-2-(3-hydroxy-3-methylbut-1-yn-1-yl)-5,6-dihydro-4H-benzo[de][1,6]naphthyridin-4-yl)-1-(methyl-d₃)-5-(trifluoromethyl)pyrrolidin-2-one

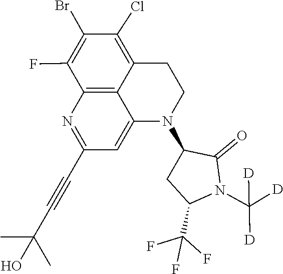 [SMILES: [2H]C([2H])([2H])N1C(=O)[C@H](N2CCc3c(Cl)c(Br)c(F)c4nc(C#CC(C)(C)O)cc2c34)C[C@H]1C(F)(F)F]

To a mixture of Intermediate 13 (52 mg, 0.087 mmol), CuI (5 mg, 0.03 mmol) and bis(triphenylphosphine)palladium dichloride (12 mg, 0.017 mmol) were added DIPEA (46 μL, 0.26 mmol), 2-methyl-3-butyn-2-ol (25 μL, 0.26 mmol) and DMF (875 μL). The reaction flask was evacuated, back filled with N₂, then stirred at r.t. for 2 h. The mixture was diluted with EtOAc and sat. aq. NaHCO₃. The layers were separated and the organic layer was washed with brine, dried over Na₂SO₄ and concentrated. The crude product was purified by FCC (0-100% EtOAc in hexanes) to provide the title compound (24 mg, 49%). LCMS calc. for C₂₂H₁₇D₃BrClF₄N₃O₂ (M+H)⁺ m/z=551.0, 553.0; found 551.0, 553.0.

### Step 2. 2-Amino-4-(7-chloro-9-fluoro-2-(3-hydroxy-3-methylbut-1-yn-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (18 mg, 0.086 mmol), tert-butyl (3-cyano-4-(5,5-dimethyl-1,3,2-dioxaborinan-2-yl)-7-fluorobenzo[b]thiophen-2-yl)carbamate (35 mg, 0.086 mmol), (3R,5S)-3-(8-bromo-7-chloro-9-fluoro-2-(3-hydroxy-3-methylbut-1-yn-1-yl)-5,6-dihydro-4H-benzo[de][1,6]naphthyridin-4-yl)-1-(methyl-d₃)-5-(trifluoromethyl)pyrrolidin-2-one (24 mg, 0.043 mmol), (2R,3R)-BaryPhos (4.5 mg, 8.63 μmol), and Pd₂(dba)₃ (5 mg, 7.76 μmol) was added DCE (400 μL) under N₂. The mixture was warmed to 50° C. for 1 h. The mixture was cooled to r.t., diluted with EtOAc and filtered. The filtrate was concentrated and the residue was purified by FCC (0-100% EtOAc in hexanes). The intermediate was dissolved in 1:1 TFA/DCM (1 mL) and the mixture was stirred at r.t. for 30 min., then diluted with MeCN and purified by prep. HPLC (XBRIDGE® C₁₈ column, eluting with a gradient of MeCN/water containing 0.1% TFA, at flow rate of 60 mL/min.) to afford the title compound. LCMS calc. for C₃₁H₂₁D₃ClF₅N₅O₂S (M+H)⁺ m/z=663.1; found 663.1.

The compounds are understood to comprise atropisomers, i.e., (Rₐ)-2-amino-4-(7-chloro-9-fluoro-2-(3-hydroxy-3-methylbut-1-yn-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-hydroxy-3-methylbut-1-yn-1-yl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-(3-hydroxy-3-methylbut-1-yn-1-yl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-hydroxy-3-methylbut-1-yn-1-yl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of the reactant of step 1 as starting material.

Further isomers are prepared by using modifications to the procedure and employing appropriate starting materials, i.e., (Rₐ)-2-amino-4-(7-chloro-9-fluoro-2-(3-hydroxy-3-methylbut-1-yn-1-yl)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-(3-hydroxy-3-methylbut-1-yn-1-yl)-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-(3-hydroxy-3-methylbut-1-yn-1-yl)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(3-hydroxy-3-methylbut-1-yn-1-yl)-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 74. 2-Amino-4-(7-chloro-2-(3-(dimethylamino)prop-1-yn-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

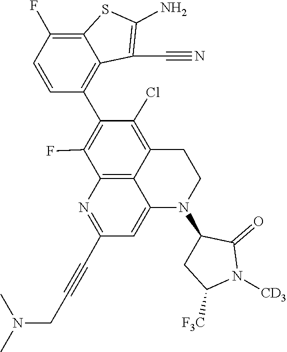 [SMILES: [2H]C([2H])([2H])N1C(=O)[C@H](N2CCc3c(Cl)c(-c4ccc(F)c5sc(N)c(C#N)c45)c(F)c4nc(C#CCN(C)C)cc2c34)C[C@H]1C(F)(F)F]

The title compound was prepared according to the general procedures outlined for Example 73, utilizing N,N-dimethylprop-2-yn-1-amine as the coupling partner in Step 1. The compound was isolated as two peaks after prep. LCMS. LCMS calc. for C₃₁H₂₂D₃ClF₅N₆OS (M+H)⁺ m/z=662.1; found 662.1.

The compounds are understood to comprise atropisomers, i.e., (Rₐ)-2-amino-4-(7-chloro-2-(3-(dimethylamino)prop-1-yn-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)prop-1-yn-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-(3-(dimethylamino)prop-1-yn-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)prop-1-yn-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of the reactant of step 1 as starting material.

Further isomers are prepared by using modifications to the procedure and employing appropriate starting materials, i.e., (Rₐ)-2-amino-4-(7-chloro-2-(3-(dimethylamino)prop-1-yn-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)prop-1-yn-1-yl)-9-fluoro-4-((3R,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-(3-(dimethylamino)prop-1-yn-1-yl)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-(dimethylamino)prop-1-yn-1-yl)-9-fluoro-4-((3S,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example 75a, 75b, 75c, and 75d

Compounds in Table 9 were prepared according to the general procedure described for Example 41, using the appropriate lactam starting material.

TABLE 9

| # | Structure | Name | [M + H] |
|---|---|---|---|
| 75a/b/c/d | 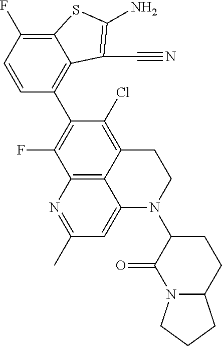 [SMILES: Cc1cc2c3c(c(Cl)c(-c4ccc(F)c5sc(N)c(C#N)c45)c(F)c3n1)CCN2C1CCC2CCCN2C1=O] | 2-amino-4-(7-chloro-9-fluoro-2-methyl- 4-(5-oxooctahydroindolizin-6-yl)-5,6- dihydro-4H-benzo[de][1,6]naphthyridin- 8-yl)-7-fluorobenzo[b]thiophene-3- carbonitrile | 564.2 |

The compounds of Example 75a, 75b, 75c and 75d are understood to comprise mixtures of diastereoisomers and/or atropisomers, i.e., (Rₐ)-2-amino-4-(7-chloro-9-fluoro-2-methyl-4-((6S,8aR)-5-oxooctahydroindolizin-6-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((6S,8aR)-5-oxooctahydroindolizin-6-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((6S,8aS)-5-oxooctahydroindolizin-6-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((6S,8aS)-5-oxooctahydroindolizin-6-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((6R,8aR)-5-oxooctahydroindolizin-6-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((6R,8aR)-5-oxooctahydroindolizin-6-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((6R,8aS)-5-oxooctahydroindolizin-6-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((6R,8aS)-5-oxooctahydroindolizin-6-yl)-5,6-dihydro-4H-benzo[de][1,6]naphthyridin-8-yl)-7-fluorobenzo[b]thiophene-3-carbonitrile.

### Example 76-78

Compounds in Table 10 were prepared according to the general procedure described for Example 48, using the appropriate amine starting material.

TABLE 10

| # | Structure | Name | [M + H] |
|---|---|---|---|
| 76 | 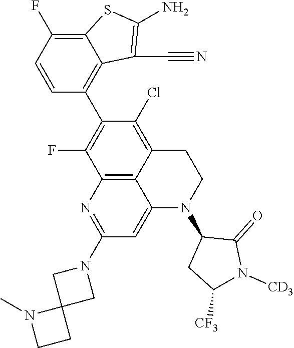 [SMILES: [2H]C([2H])([2H])N1C(=O)[C@H](N2CCc3c(Cl)c(-c4ccc(F)c5sc(N)c(C#N)c45)c(F)c4nc(N5CC6(CCN6C)C5)cc2c34)C[C@H]1C(F)(F)F] | 2-amino-4-(7-chloro-9-fluoro-4- ((3R,5S)-1-(methyl-d₃)-2-oxo-5- (trifluoromethyl)pyrrolidin-3-yl)-2-(1- methyl-1,6-diazaspiro[3.3]heptan-6- yl)-5,6-dihydro-4H- benzo[de][1,6]naphthyridin-8-yl)-7- fluorobenzo[b]thiophene-3- carbonitrile | 691.2 |
| 77a/b/c/d | 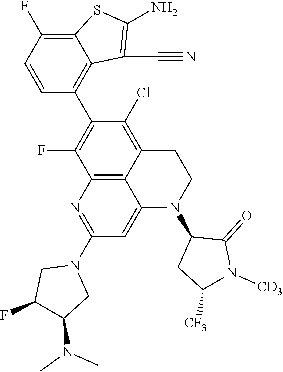 [SMILES: [2H]C([2H])([2H])N1C(=O)[C@H](N2CCc3c(Cl)c(-c4ccc(F)c5sc(N)c(C#N)c45)c(F)c4nc(N5C[C@@H](N(C)C)[C@@H](F)C5)cc2c34)C[C@H]1C(F)(F)F] | 2-amino-4-(7-chloro-2-(3- (dimethylamino)-4-fluoropyrrolidin-1- yl)-9-fluoro-4-((3R,5S)-1-(methyl-d₃)- 2-oxo-5-(trifluoromethyl)pyrrolidin-3- yl)-5,6-dihydro-4H- benzo[de][1,6]naphthyridin-8-yl)-7- fluorobenzo[b]thiophene-3- carbonitrile | 711.2 |
| 78a/b | 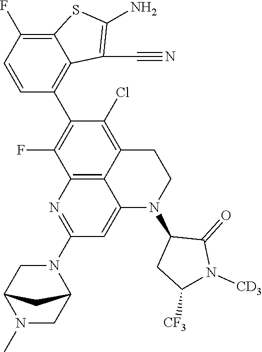 [SMILES: [2H]C([2H])([2H])N1C(=O)[C@H](N2CCc3c(Cl)c(-c4ccc(F)c5sc(N)c(C#N)c45)c(F)c4nc(N5C[C@@H]6C[C@H]5CN6C)cc2c34)C[C@H]1C(F)(F)F] | 2-amino-4-(7-chloro-9-fluoro-4- ((3R,5S)-1-(methyl-d₃)-2-oxo-5- (trifluoromethyl)pyrrolidin-3-yl)-2-(5- methyl-2,5-diazabicyclo[2.2.1]heptan- 2-yl)-5,6-dihydro-4H- benzo[de][1,6]naphthyridin-8-yl)-7- fluorobenzo[b]thiophene-3- carbonitrile | 691.2 |

### Example 76

The compound of Example 76 is understood to comprise a mixture of atropisomers, i.e., (Rₐ)-2-amino-4-(7-chloro-9-fluoro-4-((3R,5S)-1-(methyl-d₃)-2-oxo-5-(trifluoromethyl) pyrrolidin-3-yl)-2-(1-methyl-1,6-diazaspiro[3.3]heptan-6-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-(1-methyl-1,6-diazaspiro[3.3]heptan-6-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-(1-methyl-1,6-diazaspiro[3.3]heptan-6-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-(1-methyl-1,6-diazaspiro[3.3]heptan-6-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 13 as starting material.

### Examples 77a, 77b, 77c, and 77d

The compounds of Example 77a, 77b, 77c, and 77d are understood to comprise atropisomers, i.e., (Rₐ)-2-amino-4-(7-chloro-2-((3R,4S)-3-(dimethylamino)-4-fluoropyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((3R,4S)-3-(dimethylamino)-4-fluoropyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((3S,4R)-3-(dimethylamino)-4-fluoropyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3S,4R)-3-(dimethylamino)-4-fluoropyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((3R,4S)-3-(dimethylamino)-4-fluoropyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((3R,4S)-3-(dimethylamino)-4-fluoropyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((3S,4R)-3-(dimethylamino)-4-fluoropyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3S,4R)-3-(dimethylamino)-4-fluoropyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile are prepared by an analogous procedure using the alternative diastereomer of Intermediate 13 as starting material.

### Examples 78a and 78b

The compounds of Example 78a and 78b are understood to comprise mixtures of diasteromers and/or atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-2-((1 S,4S)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-((1 S,4S)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-((1R,4R)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-((1R,4R)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-((1S,4S)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-((1 S,4S)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-((1R,4R)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-((1R,4R)-5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile, are prepared by an analogous procedure using the 25 alternative diastereomer of Intermediate 13 as starting material.

### Example 79a and 79b. 2-Amino-4-(7-chloro-9-fluoro-2-methyl-4-((3R,5S)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile

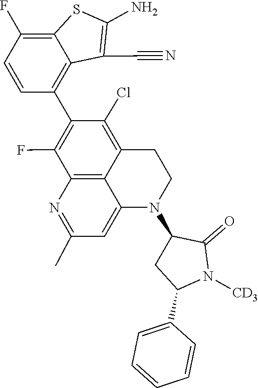 [SMILES: [2H]C([2H])([2H])N1C(=O)[C@H](N2CCc3c(Cl)c(-c4ccc(F)c5sc(N)c(C#N)c45)c(F)c4nc(C)cc2c34)C[C@H]1c1ccccc1]

### Step 1. (S)-1-(Methyl-d₃)-5-phenylpyrrolidin-2-one

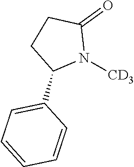 [SMILES: [2H]C([2H])([2H])N1C(=O)CC[C@H]1c1ccccc1]

To the mixture of (S)-5-phenylpyrrolidin-2-one (500 mg, 3.10 mmol) and Cs₂CO₃ (2.02 g, 6.20 mmol) in MeCN (12.4 mL) was added iodomethane-d₃ (386 μL, 6.20 mmol) at r.t. and the reaction mixture was heated at 70° C. until full conversion was indicated by LCMS. The mixture was diluted with DCM and filtered through a plug of diatomaceous earth. The frit was washed with additional DCM. The filtrate was concentrated, the crude product was purified by FCC (0-100% EtOAc in hexane) to provide the title compound. LCMS calc. for C₁₁H₁₁D₃NO (M+H)⁺ m/z=179.1; found 179.1.

### Step 2. (3R,5S)-3-Amino-1-(methyl-d₃)-5-phenylpyrrolidin-2-one

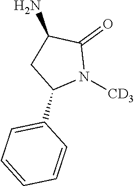 [SMILES: [2H]C([2H])([2H])N1C(=O)[C@H](N)C[C@H]1c1ccccc1]

To a solution of (S)-1-(methyl-d₃)-5-phenylpyrrolidin-2-one (200 mg, 1.12 mmol) in THF (5.6 mL) at −78° C. was added LDA (2.0 M in THF/heptane/ethylbenzene (617 μL, 1.23 mmol). The resulting mixture was stirred at −78° C. for 30 min. before 2,4,6-triisopropylbenzenesulfonyl azide (382 mg, 1.23 mmol) was added at −78° C. The reaction mixture was then allowed to warm slowly to r.t. The reaction was quenched with AcOH (128 μL, 2.24 mmol), and the mixture was diluted with EtOAc/brine and separated. The organic layer was dried over Na₂SO₄ and concentrated. The crude product was purified by FCC (0-100% EtOAc in hexane). The purified product was then dissolved in THF/water and stirred with triphenylphosphine (294 mg, 1.12 mmol) until full conversion. The mixture was then diluted with MeCN and purified by prep. HPLC (XBRIDGE® C₁₈ column, eluting with a gradient of MeCN/water containing 0.1% TFA, at flow rate of 60 mL/min.) to afford the title compound. LCMS calc. for C₁₁H₁₂D₃N₂O (M+H)⁺ m/z=194.1; found 194.1.

### Step 3. (3R,5S)-3-(8-bromo-7-chloro-9-fluoro-2-methyl-5,6-dihydro-4H-benzo[de][1,6]naphthyridin-4-yl)-1-(methyl-d₃)-5-phenylpyrrolidin-2-one

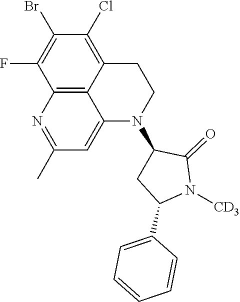 [SMILES: [2H]C([2H])([2H])N1C(=O)[C@H](N2CCc3c(Cl)c(Br)c(F)c4nc(C)cc2c34)C[C@H]1c1ccccc1]

To the solution of 2-(7-bromo-4,6-dichloro-8-fluoro-2-methylquinolin-5-yl)acetaldehyde (Intermediate 2, 30 mg, 0.085 mmol) in DCM (427 μL) were added (3R,5S)-3-amino-1-(methyl-d₃)-5-phenylpyrrolidin-2-one (24.8 mg, 0.128 mmol), PPTS (21.5 mg, 0.085 mmol), and NaBH(OAc)₃ (36.2 mg, 0.171 mmol), and the reaction mixture was stirred at r.t. for 2 h. The reaction was quenched with sat. aq. NaHCO₃ and the mixture was extracted with DCM. The organic layer was dried over Na₂SO₄ and concentrated. The crude product was purified by FCC (0-100% EtOAc in hexane) to provide the title compound (41 mg, 98%). LCMS calc. for C₂₃H₁₃D₃BrClFN₃O (M+H)⁺ m/z=491.1, 493.1; found 491.1, 493.1.

### Step 4. 2-amino-4-(7-chloro-9-fluoro-2-methyl-4-((3R,5S)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile

In a 1 dram vial was added tert-butyl (3-cyano-4-(5,5-dimethyl-1,3,2-dioxaborinan-2-yl)-7-fluorobenzo[b]thiophen-2-yl)carbamate (84 mg, 0.208 mmol), (3R,5S)-3-(8-bromo-7-chloro-9-fluoro-2-methyl-5,6-dihydro-4H-benzo[de][1,6]naphthyridin-4-yl)-1-(methyl-d₃)-5-phenylpyrrolidin-2-one (41 mg, 0.083 mmol), and dichloro[bis(diphenylphosphinophenyl)ether]palladium(II) (17.9 mg, 0.025 mmol). The reaction flask was evacuated, back filled with N₂ before 1,4-dioxane (834 μL) was added and then stirred at 100° C. for 1 h. The reaction was quenched with water and extracted with EtOAc. The organic layer was washed with brine, dried over Na₂SO₄ and concentrated. The crude product was dissolved in 1:1 DCM/TFA, and the mixture was stirred at r.t. for 30 min. The mixture was concentrated, then diluted with MeCN and purified by prep. HPLC (XBRIDGE® C₁₈ column, eluting with a gradient of MeOH/water containing 0.1% NH₄OH, at flow rate of 60 mL/min.) to afford the title compound as two peaks.

Example 79a. Diastereomer 1. Peak 1. LCMS calc. for C₃₂H₂₂D₃ClF₂N₅OS (M+H)⁺ m/z=603.2; found 603.2. This is the potent peak.

Example 79b. Diastereomer 2. Peak 2. LCMS calc. for C₃₂H₂₂D₃ClF₂N₅OS (M+H)⁺ m/z=603.2; found 603.2.

The compounds are understood to comprise atropisomers, i.e.:
(Rₐ)-2-amino-4-(7-chloro-9-fluoro-2-methyl-4-((3R,5S)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile; and
(Sₐ)-2-amino-4-(7-chloro-9-fluoro-2-methyl-4-((3R,5S)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile.

The alternative isomers, i.e.:
(Rₐ)-2-amino-4-(7-chloro-9-fluoro-2-methyl-4-((3S,5R)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile; and
(Sₐ)-2-amino-4-(7-chloro-9-fluoro-2-methyl-4-((3S,5R)-1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile;

are prepared by an analogous procedure using methyl (R)-5-phenylpyrrolidin-2-one as starting material.

### Example 80a and 80b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile

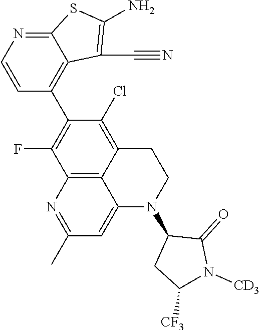 [SMILES: [2H]C([2H])([2H])N1C(=O)[C@H](N2CCc3c(Cl)c(-c4ccnc5sc(N)c(C#N)c45)c(F)c4nc(C)cc2c34)C[C@H]1C(F)(F)F]

### Step 1. (4-Bromo-2-fluoropyridin-3-yl)methanol

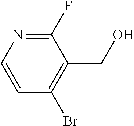

To solution of 4-bromo-2-fluoronicotinaldehyde (5.0 g, 24.5 mmol) in THF (61 mL) at 0° C. was added NaBH₄ (1.39 g, 36.8 mmol). The mixture was then stirred at r.t. for 30 min., then quenched with aq. NH₄Cl. The mixture was diluted with EtOAc and extract, organic layer was separated, washed with brine, dried with Na₂SO₄ and concentrated. The crude product was used directly without further purification. LCMS calc. for C₆H₆BrFNO (M+H)⁺ m/z=206.0, 208.0; found 206.0, 208.0.

### Step 2. (4-Bromo-2-fluoropyridin-3-yl)methyl methanesulfonate

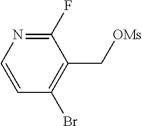 [SMILES: CS(=O)(=O)OCc1c(Br)ccnc1F]

To a solution of (4-bromo-2-fluoropyridin-3-yl)methanol (5.05 g, 24.5 mmol) in THF (61 mL) was added DIPEA (5.1 mL, 29.4 mmol) and the reaction mixture was cooled to 0° C. Methanesulfonic anhydride (4.70 g, 27.0 mmol) was then added and the reaction mixture was allowed to warm to r.t. overnight. The reaction mixture was diluted with EtOAc/tert-butyl ether (1:1) and washed with cold water. The layers were separated and the organic layer was washed with water and brine, dried over MgSO₄, filtered, and concentrated. The crude product was used in the next step without further purification. LCMS calc. for C₇H₃BrFNO₃S (M+H)⁺ m/z=283.9, 285.9; found 283.9, 285.9.

### Step 3. 2-(4-Bromo-2-fluoropyridin-3-yl)acetonitrile

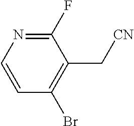 [SMILES: N#CCc1c(Br)ccnc1F]

To a solution of (4-bromo-2-fluoropyridin-3-yl)methyl methanesulfonate (5.6 g, 19.7 mmol) in EtOH (54.8 mL)/water (11.0 mL) was added KCN (1.41 g, 21.7 mmol) and the reaction mixture was heated at 60° C. for 10 min. After cooling to r.t., the mixture was concentrated, then diluted with DCM/water. The layers were separated, and the organic layer was washed with sat. aq. NaHCO₃ and brine, dried over Na₂SO₄ and concentrated. The crude product was purified by FCC (0-40% EtOAc in hexanes) to provide the title compound (1.6 g, 38%). LCMS calc. for C₇H₅BrFN₂ (M+H)⁺ m/z=215.0, 217.0; found 215.0, 217.0.

### Step 4. Ethyl (4-bromo-3-cyanothieno[2,3-b]pyridin-2-yl)carbamate

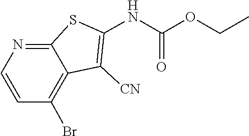 [SMILES: CCOC(=O)Nc1sc2nccc(Br)c2c1C#N]

To solution of 2-(4-bromo-2-fluoropyridin-3-yl)acetonitrile (1.60 g, 7.44 mmol) in DMF (18.6 mL) at 0° C. was added KOtBu (877 mg, 7.81 mmol). The mixture was stirred at 0° C. for 10 min. before ethoxy carbonyl isothiocyanate (912 μL, 7.81 mmol) was added and the mixture was allowed to warm to r.t. After stirring for 1 h, mixture was heated at 100° C. for 30 min. The mixture was allowed to cool to r.t. and water was added. The resulting precipitate was collected by filtration, washed with cold water and hexanes, then air dried and used in the next step without further purification. LCMS calc. for C₁₁H₅BrN₃O₂S (M+H)⁺ m/z=326.0, 328.0; found 326.0, 328.0.

### Step 5. 2-Amino-4-bromothieno[2,3-b]pyridine-3-carbonitrile

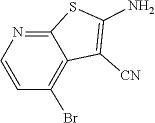 [SMILES: N#Cc1c(N)sc2nccc(Br)c12]

To a solution of ethyl (4-bromo-3-cyanothieno[2,3-b]pyridin-2-yl)carbamate (2.0 g, 6.13 mmol) in DMSO (8.8 mL) was added NaOH (5.0 M, 6.7 mL, 33.7 mmol) and the mixture was stirred at 100° C. for 3 h. The mixture was cooled to r.t. and diluted with water. The resulting precipitate was collected by filtration, washed with water and hexanes, then dried to give the title compound (676 mg, 43%), which was used in the next step without further purification. LCMS calc. for C₃H₅BrN₃S (M+H)⁺ m/z=254.0, 256.0; found 254.0, 256.0.

### Step 6. tert-Butyl (4-bromo-3-cyanothieno[2,3-b]pyridin-2-yl)carbamate

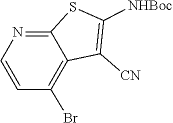 [SMILES: CC(C)(C)OC(=O)Nc1sc2nccc(Br)c2c1C#N]

To a solution of 2-amino-4-bromothieno[2,3-b]pyridine-3-carbonitrile (676 mg, 2.66 mmol) in THF (13.3 mL) were added DMAP (13.0 mg, 0.106 mmol), DIPEA (558 μL, 3.19 mmol) and Boc₂O (581 mg, 2.66 mmol). The reaction mixture was stirred at r.t. for 1 h then heated at 40° C. for 4 h. The mixture was diluted with water and filtered. The filtrate was washed with water and air dried to provide the title compound (700 mg, 74%), which was used in the next step without further purification. LCMS calc. for C₁₃H₁₃BrN₃O₂S (M+H)⁺ m/z=354.0, 356.0; found 354.0, 356.0.

### Step 7. tert-Butyl (4-bromo-3-cyanothieno[2,3-b]pyridin-2-yl)((2-(trimethylsilyl)ethoxy)methyl)carbamate

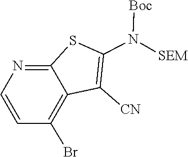 [SMILES: CC(C)(C)OC(=O)N(COCC[Si](C)(C)C)c1sc2nccc(Br)c2c1C#N]

To a solution of tert-butyl (4-bromo-3-cyanothieno[2,3-b]pyridin-2-yl)carbamate (700 mg, 1.98 mmol) in DMF (9.9 mL) at 0° C. was added NaH (60% w/w mineral oil, 87 mg, 2.17 mmol) and the mixture was then stirred at r.t. for 30 min. The mixture was cooled to 0° C. SEMCl (466 μL, 2.37 mmol) was added and the mixture was allowed to warm to r.t., then quenched with sat. NaHCO₃. The mixture was extracted with EtOAc. The organic layer was washed with water and brine, dried over Na₂SO₄ and concentrated. The crude product was purified by FCC (0-50% EtOAc in hexanes) to provide the title compound (700 mg, 73%). LCMS calc. for C₁₉H₂₇BrN₃O₃SSi (M+H)⁺ m/z=484.1, 486.1; found 484.1, 486.1.

### Step 8. tert-Butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyanothieno[2,3-b]pyridin-2-yl)((2-(trimethylsilyl)ethoxy)methyl)carbamate

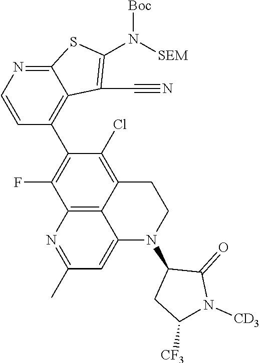 [SMILES: [2H]C([2H])([2H])N1C(=O)[C@H](N2CCc3c(Cl)c(-c4ccnc5sc(N(COCC[Si](C)(C)C)C(=O)OC(C)(C)C)c(C#N)c45)c(F)c4nc(C)cc2c34)C[C@H]1C(F)(F)F]

To a solution of (3R,5S)-3-(8-bromo-7-chloro-9-fluoro-2-methyl-5,6-dihydro-4H-benzo[de][1,6]naphthyridin-4-yl)-1-(methyl-d₃)-5-(trifluoromethyl)pyrrolidin-2-one (Intermediate 12, 100 mg, 0.207 mmol) in THF (1.3 mL) at −78° C. was added iPrMgCl·LiCl (1.3 M in THF, 334 μL, 0.434 mmol). The resulting solution was stirred for 45 min. at −78° C. before ZnCl₂ (1.9 M in 2-Me-THF, 228 μL, 0.434 mmol) was added dropwise. After stirring at −78° C. for an additional 10 min., the solution was allowed to warm to r.t. and stirred for 30 min. The solution was transferred to a solution of tert-butyl (4-bromo-3-cyano-5-fluorobenzo[b]thiophen-2-yl)((2-(trimethylsilyl)ethoxy)methyl)carbamate (110 mg, 0.227 mmol) and RuPhos-Pd G4 (33.3 mg, 0.041 mmol) in THF (1.0 mL) and the resulting mixture heated at 60° C. overnight. After cooling to r.t., the reaction mixture was quenched with sat. aq. NaHCO₃ and extracted with EtOAc. The organic layer was dried over Na₂SO₄ and concentrated. The crude product was purified by prep. HPLC (XBRIDGE® C₁₈ column, eluting with a gradient of MeCN/water containing 0.1% NH₄OH, at flow rate of 60 mL/min.) to afford the title compound (61 mg, 37%). LCMS calc. for C₃₇H₃₉D₃ClF₄N₆O₄SSi (M+H)⁺ m/z=808.2; found 808.2.

### Step 9. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile

A solution of tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyanothieno[2,3-b]pyridin-2-yl)((2-(trimethylsilyl)ethoxy)methyl)carbamate (61 mg, 0.075 mmol) in (1:1) DCM/TFA was stirred at r.t. for 1 h, then concentrated, diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 80a. Diastereomer 1. Peak 1. LCMS calc. for C₂₆H₁₇D₃ClF₄N₆OS (M+H)⁺ m/z=578.1; found 578.1. This is the potent peak.

Example 80b. Diastereomer 2. Peak 2. LCMS calc. for C₂₆H₁₇D₃ClF₄N₆OS (M+H)⁺ m/z=578.1; found 578.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile; are prepared by an analogous procedure using the alternative diastereomer of Intermediate 11 as starting material.

### Example 81a and 81b. 2-Amino-4-(7-chloro-2-((dimethyl(oxo)-λ⁶-sulfaneylidene)amino)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

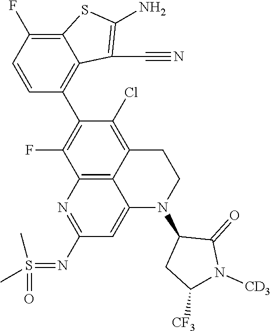 [SMILES: [2H]C([2H])([2H])N1C(=O)[C@H](N2CCc3c(Cl)c(-c4ccc(F)c5sc(N)c(C#N)c45)c(F)c4nc(N=S(C)(C)=O)cc2c34)C[C@H]1C(F)(F)F]

### Step 1. (3R,5S)-3-(8-Bromo-7-chloro-2-((dimethyl(oxo)-λ⁶-sulfaneylidene)amino)-9-fluoro-5,6-dihydro-4H-benzo[de][1,6]naphthyridin-4-yl)-1-(methyl-d₃)-5-(trifluoromethyl)pyrrolidin-2-one

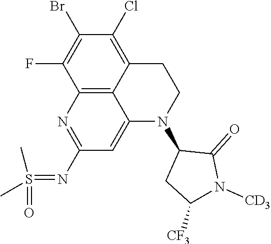 [SMILES: [2H]C([2H])([2H])N1C(=O)[C@H](N2CCc3c(Cl)c(Br)c(F)c4nc(N=S(C)(C)=O)cc2c34)C[C@H]1C(F)(F)F]

In a 1 dram vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 40 mg, 0.067 mmol), iminodimethyl-λ⁶-sulfanone (31.3 mg, 0.336 mmol), L-proline (6.8 mg, 0.059 mmol), CuI (6.4 mg, 0.034 mmol), and K₂CO₃ (46.4 mg, 0.336 mmol) were dissolved in DMSO (448 μL) under N₂. The reaction mixture was heated at 85° C. for 15 min, then diluted with 30% aq. NH₄OH, and extracted with DCM. The organic layer was washed with 10% aq. LiCl, dried over MgSO₄ and concentrated. The crude product was used for the next step without further purification. LCMS calc. for C₁₉H₁₆D₃BrClF₄N₄O₂S (M+H)⁺ m/z=560.0, 562.0; found 560.0, 562.0.

### Step 2. 2-Amino-4-(7-chloro-2-((dimethyl(oxo)-λ⁶-sulfaneylidene)amino)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-2-((dimethyl(oxo)-λ⁶-sulfaneylidene)amino)-9-fluoro-5,6-dihydro-4H-benzo[de][1,6]naphthyridin-4-yl)-1-(methyl-d₃)-5-(trifluoromethyl)pyrrolidin-2-one (37.7 mg, 0.067 mmol), tert-butyl (3-cyano-4-(5,5-dimethyl-1,3,2-dioxaborinan-2-yl)-7-fluorobenzo[b]thiophen-2-yl)carbamate (67.9 mg, 0.168 mmol), dichloro [bis(diphenylphosphinophenyl)ether]palladium(II) (12.0 mg, 17 μmol), and K₂CO₃ (37.2 mg, 0.269 mmol) were dissolved in 1,4-dioxane (672 μL) under N₂. The reaction mixture was stirred at 100° C. for 30 min., then diluted with MeCN and filtered. The filtrate was concentrated and dissolved in 1:1 DCM/TFA. After stirring at r.t. for 30 min., the mixture was concentrated, then diluted with MeCN and purified by prep. HPLC (XBRIDGE® Cl₈ column, eluting with a gradient of MeCN/water containing 0.1% NH₄OH, at flow rate of 60 mL/min.) to afford the title compound as two peaks.

Example 81a. Diastereomer 1. Peak 1. LCMS calc. for C₂₈H₂₀D₃ClF₅N₆O₂S₂(M+H)⁺ m/z=672.1; found 672.1. This is the potent peak.

Example 81b. Diastereomer 2. Peak 2. LCMS calc. for C₂₈H₂₀D₃ClF₅N₆O₂S₂(M+H)⁺ m/z=672.1; found 672.1.

The compounds are understood to comprise atropisomers, i.e., (Rₐ)-2-amino-4-(7-chloro-2-((dimethyl(oxo)-λ⁶-sulfaneylidene)amino)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((dimethyl(oxo)-λ⁶-sulfaneylidene)amino)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((dimethyl(oxo)-λ⁶-sulfaneylidene)amino)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((dimethyl(oxo)-λ⁶-sulfaneylidene)amino)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 82. 2-Amino-4-(7-chloro-2-((S)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

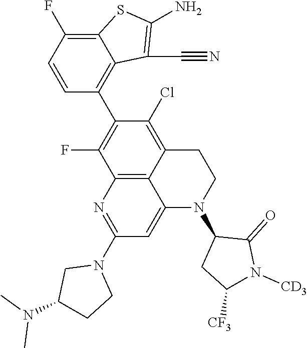 [SMILES: [2H]C([2H])([2H])N1C(=O)[C@H](N2CCc3c(Cl)c(-c4ccc(F)c5sc(N)c(C#N)c45)c(F)c4nc(N5CC[C@H](N(C)C)C5)cc2c34)C[C@H]1C(F)(F)F]

### Step 1. (3R,5S)-3-(8-Bromo-7-chloro-2-((S)-3-(dimethylamino)pyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one

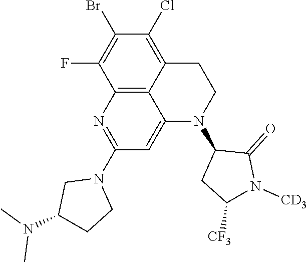 [SMILES: [2H]C([2H])([2H])N1C(=O)[C@H](N2CCc3c(Cl)c(Br)c(F)c4nc(N5CC[C@H](N(C)C)C5)cc2c34)C[C@H]1C(F)(F)F]

In a 40 mL vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 1.12 g, 1.88 mmol), (S)—N,N-dimethylpyrrolidin-3-amine (1.07 g, 9.41 mmol), L-proline (54 mg, 0.47 mmol), CuI (90 mg, 0.47 mmol) and K₂CO₃ (1.30 g, 9.41 mmol) were dissolved in DMSO (9.4 mL) under N₂. The reaction mixture was heated at 85° C. for 15 min., then diluted with DCM and 30%. aq. NH₄OH. The organic layer was dried over MgSO₄ and concentrated. The crude product was used in the next step without further purification. LCMS calc. for C₂₃H₂₃D₃BrClF₄N₅O (M+H)⁺ m/z=581.1, 583.1; found 581.1, 583.1.

### Step 2. 2-Amino-4-(7-chloro-2-((S)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 40 mL vial, (3R,5S)-3-(8-bromo-7-chloro-2-((S)-3-(dimethylamino)pyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one (997 mg, 1.71 mmol), dichloro[bis (diphenylphosphinophenyl)ether]palladium(II) (307 mg, 0.428 mmol), and K₂CO₃ (710 mg, 5.14 mmol) were dissolved in 1,4-dioxane (6.9 mL) under N₂. The reaction mixture was stirred at 100° C. for 1 h, then diluted with EtOAc and filtered over diatomaceous earth. The volatiles were removed under vacuum and the crude residue was purified by FCC (0-25% MeOH in DCM). The product was dissolved in TFA and the solution was stirred at r.t., then concentrated, diluted with MeCN, and purified by prep. HPLC (XBRIDGE® C₁₈ column, eluting with a gradient of MeCN/water containing 0.1% NH₄OH, at flow rate of 60 mL/min.) to afford the title compound as two peaks.

Example 82. Diastereomer 1. Peak 1. LCMS calc. for C₃₂H₂₇D₃ClF₅N₇OS (M+H)⁺ m/z=693.2; found 693.2. This is the potent peak. ¹H NMR (500 MHz, DMSO) b 8.01 (s, 2H), 7.21 (dd, J=8.4, 5.3 Hz, 1H), 7.13 (t, J=8.9 Hz, 1H), 5.98 (s, 1H), 5.10 (t, J=9.2 Hz, 1H), 4.65 (td, J=8.5, 3.6 Hz, 1H), 4.10-3.97 (m, 2H), 3.84 (td, J=9.9, 3.7 Hz, 1H), 3.67 (dd, J=11.2, 5.9 Hz, 1H), 3.52 (dd, J=10.2, 7.5 Hz, 1H), 3.42 (dt, J=12.6, 6.3 Hz, 1H), 3.25 (dt, J=12.1, 6.1 Hz, 1H), 3.13-2.99 (m, 2H), 2.88 (s, 6H), 2.65-2.54 (m, 2H), 2.49-2.43 (m, 1H), 2.24 (dq, J=12.6, 8.4, 1H).

The compounds are understood to comprise atropisomers, i.e., (Rₐ)-2-amino-4-(7-chloro-2-((S)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((S)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((R)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((R)-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((S)-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((S)-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((R)-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((R)-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 83a, 83b, and 83c. 2-Amino-4-(7-chloro-2-(3-(dimethylamino)-3-methylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

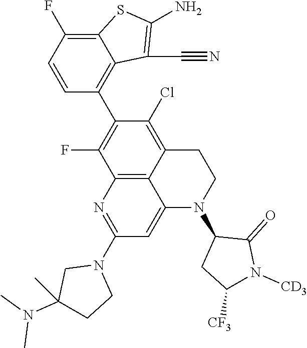 [SMILES: [2H]C([2H])([2H])N1C(=O)[C@H](N2CCc3c(Cl)c(-c4ccc(F)c5sc(N)c(C#N)c45)c(F)c4nc(N5CCC(C)(N(C)C)C5)cc2c34)C[C@H]1C(F)(F)F]

### Step 1. (3R,5S)-3-(8-Bromo-7-chloro-2-(3-(dimethylamino)-3-methylpyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one

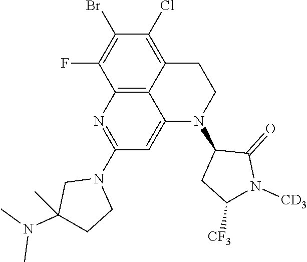 [SMILES: [2H]C([2H])([2H])N1C(=O)[C@H](N2CCc3c(Cl)c(Br)c(F)c4nc(N5CCC(C)(N(C)C)C5)cc2c34)C[C@H]1C(F)(F)F]

In a 40 mL vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 2.00 g, 3.36 mmol), N,N,3-trimethylpyrrolidin-3-amine dihydrochloride (2.60 g, 12.9 mmol), L-proline (38.7 mg, 0.336 mmol), CuI (64.0 mg, 0.336 mmol) and K₂CO₃ (2.32 g, 16.8 mmol) were dissolved in DMSO (13.4 mL) under N₂. The reaction mixture was heated at 80° C. for 30 min., then diluted with EtOAc and 30% aq. NH₄OH, and extracted with EtOAc. The organic layer was washed with 30% aq. NH₄OH/10% aq. LiCl, dried over MgSO₄ and concentrated. The crude product was redissolved in DCM, taken through a thiol cartridge, and washed with MeOH. The filtrate was concentrated and used in the next step without further purification. LCMS calc. for C₂₄H₂₅D₃BrClF₄N₅O (M+H)⁺ m/z=595.1, 597.1; found 595.1, 597.1.

### Step 2. 2-Amino-4-(7-chloro-2-(3-(dimethylamino)-3-methylpyrrolidin-1-yl)-9-fluoro-4-((3R,5S)-1-(methyl-d3)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 40 mL vial, (3R,5S)-3-(8-bromo-7-chloro-2-(3-(dimethylamino)-3-methylpyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one (658 mg, 1.10 mmol), dichloro[bis(diphenylphosphinophenyl)ether]palladium(II) (198 mg, 0.276 mmol), and K₂CO₃ (458 mg, 3.31 mmol) were dissolved in 1,4-dioxane (4.4 mL) under N₂. The reaction mixture was stirred at 100° C. for 1 h, then diluted with EtOAc and filtered through diatomaceous earth. The volatiles were removed under vacuum, and the crude residue was purified by FCC (0-25% MeOH in DCM). The product was dissolved in TFA and the solution was stirred at r.t. then concentrated, diluted with MeCN and purified by prep. HPLC (XBRIDGE® C₁₈ column, eluting with a gradient of MeCN/water containing 0.1% NH₄OH, at flow rate of 60 mL/min.) to afford the title compound as three peaks.

Example 83a. Diastereomer 1. Peak 1. LCMS calc. for C₃₃H₂₉D₃ClF₅N₇OS (M+H)⁺ m/z=707.2; found 707.2. This is the potent peak.

Example 83b. Diastereomer 2. Peak 2. LCMS calc. for C₃₃H₂₉D₃ClF₅N₇OS (M+H)⁺ m/z=707.2; found 707.2. This is the potent peak. ¹H NMR (600 MHz, DMSO) b 8.01 (s, 2H), 7.20 (dd, J=8.4, 5.3 Hz, 1H), 7.12 (t, J=8.9 Hz, 1H), 5.95 (s, 1H), 5.07 (t, J=9.2 Hz, 1H), 4.65 (t, J=8.8 Hz, 1H), 3.95-3.79 (m, 2H), 3.69-3.53 (m, 2H), 3.41-3.37 (m, 1H), 3.27-3.21 (m, 1H), 3.12-3.00 (m, 2H), 2.84 (s, 6H), 2.63-2.52 (m, 2H), 2.35-2.16 (m, 2H), 1.39 (s, 3H).

Example 83c. Diastereomer 2. Peak 2. LCMS calc. for C₃₃H₂₉D₃ClF₅N₇OS (M+H)⁺ m/z=707.2; found 707.2.

The compounds are understood to comprise atropisomers and diastereoisomers, i.e., (Rₐ)-2-amino-4-(7-chloro-2-((R)-3-(dimethylamino)-3-methylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((R)-3-(dimethylamino)-3-methylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((S)-3-(dimethylamino)-3-methylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((S)-3-(dimethylamino)-3-methylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((R)-3-(dimethylamino)-3-methylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((R)-3-(dimethylamino)-3-methylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((S)-3-(dimethylamino)-3-methylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((S)-3-(dimethylamino)-3-methylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 84. 2-Amino-4-(7-chloro-9-fluoro-4-((3R,5S)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile

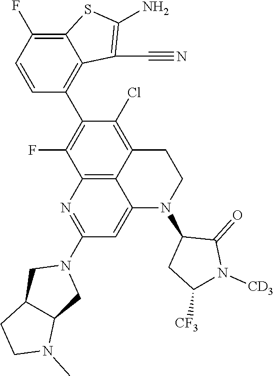 [SMILES: [2H]C([2H])([2H])N1C(=O)[C@H](N2CCc3c(Cl)c(-c4ccc(F)c5sc(N)c(C#N)c45)c(F)c4nc(N5C[C@@H]6CCN(C)[C@@H]6C5)cc2c34)C[C@H]1C(F)(F)F]

### Step 1. (3R,5S)-3-(8-Bromo-7-chloro-9-fluoro-2-((3aS,6aS)-1-methylhexahydropyrrolo[3,4-b]pyrrol-5 (1H)-yl)-5,6-dihydro-4H-benzo[de][1,6]naphthyridin-4-yl)-1-(methy-d₃)-5-(trifluoromethyl)pyrrolidin-2-one

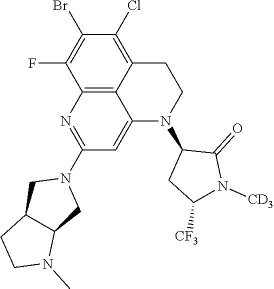 [SMILES: [2H]C([2H])([2H])N1C(=O)[C@H](N2CCc3c(Cl)c(Br)c(F)c4nc(N5C[C@@H]6CCN(C)[C@@H]6C5)cc2c34)C[C@H]1C(F)(F)F]

In a 40 mL vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 89.9 mg, 0.151 mmol), (3aS,6aS)-1-methyloctahydropyrrolo[3,4-b]pyrrole dihydrochloride (90.0 mg, 0.452 mmol), L-proline (13.9 mg, 0.121 mmol), CuI (11.5 mg, 0.060 mmol) and K₂CO₃ (192 mg, 0.905 mmol) were dissolved in DMSO (1.0 mL) under N₂. The reaction mixture was stirred at r.t. for 1 h, then diluted with 30% aq. NH₄OH, and extracted with EtOAc. The organic layer was washed with brine, dried over Na₂SO₄ and concentrated. The crude product was purified by FCC (0-100% EtOAc in hexanes) to provide the title compound (59.1 mg, 66%). LCMS calc. for C₂₄H₂₃D₃BrClF₄N₅O (M+H)⁺ m/z=593.1, 595.1; found 593.1, 595.1.

### Step 2. 2-Amino-4-(7-chloro-9-fluoro-4-((3R,5S)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile

To a mixture of K₃PO₄ (15.2 mg, 0.072 mmol), tert-butyl (3-cyano-4-(5,5-dimethyl-1,3,2-dioxaborinan-2-yl)-7-fluorobenzo[b]thiophen-2-yl)carbamate (29.0 mg, 0.072 mmol), (3R,5S)-3-(8-bromo-7-chloro-9-fluoro-2-((3aS,6aS)-1-methylhexahydropyrrolo[3,4-b]pyrrol-5 (1H)-yl)-5,6-dihydro-4H-benzo[de][1,6]naphthyridin-4-yl)-1-(methyl-d₃)-5-(trifluoromethyl)pyrrolidin-2-one (21.3 mg, 0.036 mmol), (2R,3R)-BaryPhos (3.8 mg, 7.2 μmol), and Pd(dba)₂ (3.7 mg, 6.5 μmol) was added DCE (359 μL) under N₂. The mixture was heated at 50° C. for 1 h. The mixture was cooled to r.t., diluted with EtOAc and filtered. The filtrate was concentrated, and the residue was purified by FCC (0-100% EtOAc in hexanes, then 0-10% MeOH in DCM). The intermediate was dissolved in 1:1 TFA/DCM (1 mL) and the reaction mixture was stirred at r.t. for 20 min., then diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₃₃H₂₇D₃ClF₅N₇OS (M+H)⁺ m/z=705.2; found 705.2. This is the potent peak.

The compounds are understood to comprise atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3R,5S)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3R,5S)-1-(methyl-d₃)-2-oxo-5-(trifluoromethyl)pyrrolidin-3-yl)-2-((3aR,6aR)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-((3aR,6aR)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((3S,5R)-1-(methyl-d₃)-2-oxo-5-(trifluoromethyl)pyrrolidin-3-yl)-2-((3aS,6aS)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((3S,5R)-1-(methyl-d₃)-2-oxo-5-(trifluoromethyl)pyrrolidin-3-yl)-2-((3aR,6aR)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-((3aR,6aR)-1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Examples 85-87

Compounds in Table 11 were prepared according to the general procedure described for Example 84, using the appropriate amine as starting material.

TABLE 11

| # | Structure | Name | [M + H] |
|---|---|---|---|
| 85a/b | 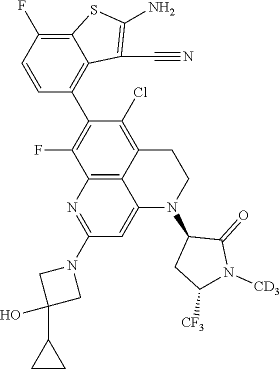 [SMILES: [2H]C([2H])([2H])N1C(=O)[C@H](N2CCc3c(Cl)c(-c4ccc(F)c5sc(N)c(C#N)c45)c(F)c4nc(N5CC(O)(C6CC6)C5)cc2c34)C[C@H]1C(F)(F)F] | 2-amino-4-(7-chloro-2-(3-cyclopropyl- 3-hydroxyazetidin-1-yl)-9-fluoro-4- ((3R,5S)-1-(methyl-d₃)-2-oxo-5- (trifluoromethyl)pyrrolidin-3-yl)-5,6- dihydro-4H- benzo[de][1,6]naphthyridin-8-yl)-7- fluorobenzo[b]thiophene-3-carbonitrile | 692.2 |
| 86a/b | 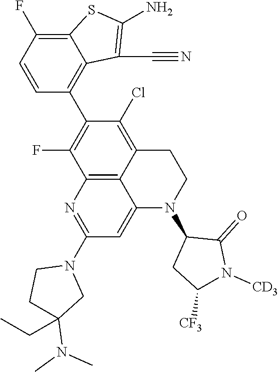 [SMILES: [2H]C([2H])([2H])N1C(=O)[C@H](N2CCc3c(Cl)c(-c4ccc(F)c5sc(N)c(C#N)c45)c(F)c4nc(N5CCC(CC)(N(C)C)C5)cc2c34)C[C@H]1C(F)(F)F] | 2-amino-4-(7-chloro-2-(3- (dimethylamino)-3-ethylpyrrolidin-1- yl)-9-fluoro-4-((3R,5S)-1-(methyl-d₃)- 2-oxo-5-(trifluoromethyl)pyrrolidin-3- yl)-5,6-dihydro-4H- benzo[de][1,6]naphthyridin-8-yl)-7- fluorobenzo[b]thiophene-3-carbonitrile | 721.2 |
| 87a/b | 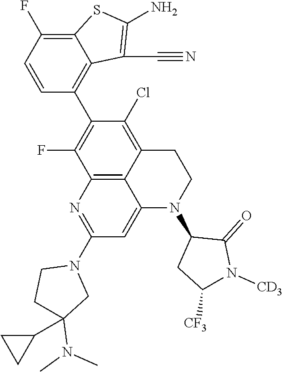 [SMILES: [2H]C([2H])([2H])N1C(=O)[C@H](N2CCc3c(Cl)c(-c4ccc(F)c5sc(N)c(C#N)c45)c(F)c4nc(N5CCC(C6CC6)(N(C)C)C5)cc2c34)C[C@H]1C(F)(F)F] | 2-amino-4-(7-chloro-2-(3-cyclopropyl- 3-(dimethylamino)pyrrolidin-1-yl)-9- fluoro-4-((3R,5S)-1-(methyl-d₃)-2-oxo- 5-(trifluoromethyl)pyrrolidin-3-yl)-5,6- dihydro-4H- benzo[de][1,6]naphthyridin-8-yl)-7- fluorobenzo[b]thiophene-3-carbonitrile | 733.2 |

The compounds of Table 11 are understood to comprise mixtures of diastereoisomers and/or atropisomers.

### Examples 85a and 85b

The compounds of Example 85a and 85b are understood to comprise atropisomers, i.e., (Rₐ)-2-amino-4-(7-chloro-2-(3-cyclopropyl-3-hydroxyazetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-cyclopropyl-3-hydroxyazetidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-(3-cyclopropyl-3-hydroxyazetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(3-cyclopropyl-3-hydroxyazetidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Examples 86a and 86b

The compounds of Example 86a and 86b are understood to comprise atropisomers and/or diastereoisomers, i.e., (Rₐ)-2-amino-4-(7-chloro-2-((R)-3-(dimethylamino)-3-ethylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((R)-3-(dimethylamino)-3-ethylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((S)-3-(dimethylamino)-3-ethylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((S)-3-(dimethylamino)-3-ethylpyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((R)-3-(dimethylamino)-3-ethylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((R)-3-(dimethylamino)-3-ethylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((S)-3-(dimethylamino)-3-ethylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((S)-3-(dimethylamino)-3-ethylpyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Examples 87a and 87b

The compounds of Example 87a and 87b are understood to comprise atropisomers 10 and/or diastereoisomers, i.e., (Rₐ)-2-amino-4-(7-chloro-2-((S)-3-cyclopropyl-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((S)-3-cyclopropyl-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((R)-3-cyclopropyl-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((R)-3-cyclopropyl-3-(dimethylamino)pyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((S)-3-cyclopropyl-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((S)-3-cyclopropyl-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((R)-3-cyclopropyl-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((R)-3-cyclopropyl-3-(dimethylamino)pyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 88a and 88b. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile

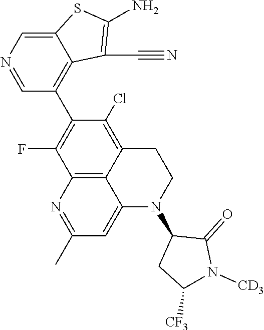 [SMILES: [2H]C([2H])([2H])N1C(=O)[C@H](N2CCc3c(Cl)c(-c4cncc5sc(N)c(C#N)c45)c(F)c4nc(C)cc2c34)C[C@H]1C(F)(F)F]

### Step 1. (3-Bromo-5-fluoropyridin-4-yl)methyl methanesulfonate

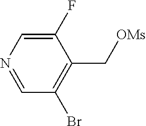 [SMILES: CS(=O)(=O)OCc1c(F)cncc1Br]

To a solution of (3-bromo-5-fluoropyridin-4-yl)methanol (5.00 g, 24.3 mmol) in THF (61 mL) was added DIPEA (5.1 mL, 29.1 mmol) and the reaction mixture was cooled to 0° C. Methanesulfonic anhydride (4.65 g, 26.7 mmol) was then added and the reaction mixture was allowed to warm to r.t. overnight. The reaction mixture was diluted with EtOAc and washed with water. The organic layer was washed with water and brine, dried over Na₂SO₄ and concentrated. The crude product was used in the next step without further purification (6.75 g, 98%). LCMS calc. for C₇H₃BrFNO₃S (M+H)⁺ m/z=283.9, 285.9; found 283.9, 285.9.

### Step 2. 2-(3-Bromo-5-fluoropyridin-4-yl)acetonitrile

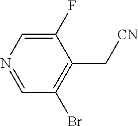 [SMILES: N#CCc1c(F)cncc1Br]

To a solution of ((3-bromo-5-fluoropyridin-4-yl)methyl methanesulfonate (6.75 g, 23.8 mmol) in EtOH (66 mL)/water (13 mL) was added KCN (1.70 g, 26.1 mmol) and the reaction mixture was heated to 50° C. for 10 min. After cooling to r.t., the mixture was concentrated, then diluted with DCM/water. The layers were separated, and the organic layer was washed with sat. NaHCO₃ and brine, dried over Na₂SO₄ and concentrated. The crude product was purified by FCC (0-40% EtOAc in hexanes) to provide the title compound (2.4 g, 47%). LCMS calc. for C₇H₅BrFN₂ (M+H)⁺ m/z=215.0, 217.0; found 215.0, 217.0.

### Step 3. Ethyl (4-bromo-3-cyanothieno[2,3-c]pyridin-2-yl)carbamate

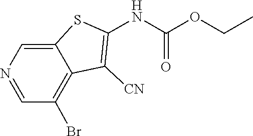 [SMILES: CCOC(=O)Nc1sc2cncc(Br)c2c1C#N]

To a 0° C. solution of 2-(3-bromo-5-fluoropyridin-4-yl)acetonitrile (2.4 g, 11.2 mmol) in DMF (27.9 mL) was added KOtBu (1.32 g, 11.7 mmol). The mixture was stirred at 0° C. for 10 min., then ethoxy carbonyl isothiocyanate (1.38 mL, 11.7 mmol) was added and the mixture was warmed to r.t., After stirring for 1 h, the reaction temperature was raised to 100° C. and stirring was continued at this temperature for 30 min. After cooling to r.t., the reaction was quenched with water. The resulting precipitate was collected by filtration, washed with cold water and hexanes, then air dried and used in the next step without further purification (2.61 g, 72%). LCMS calc. for C₁₁H₅BrN₃O₂S (M+H)⁺ m/z=326.0, 328.0; found 326.0, 328.0.

### Step 4. 2-Amino-4-bromothieno[2,3-c]pyridine-3-carbonitrile

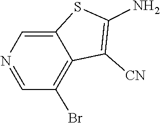 [SMILES: N#Cc1c(N)sc2cncc(Br)c12]

To a solution of ethyl (4-bromo-3-cyanothieno[2,3-c]pyridin-2-yl)carbamate (2.61 g, 8.00 mmol) in DMSO (11.4 mL) was added NaOH (5.0 M, 8.8 mL, 44 mmol) and the reaction mixture was stirred at 100° C. for 1 h. The mixture was cooled to 0° C. and diluted with water. The resulting precipitate was collected by filtration, washed with water and hexanes, then dried and used in the next step without further purification (1.85 g, 91%). LCMS calc. for C₃H₅BrN₃S (M+H)⁺ m/z=253.9, 255.9; found 253.9, 255.9.

### Step 5. tert-Butyl (4-bromo-3-cyanothieno[2,3-c]pyridin-2-yl)carbamate

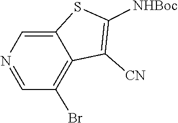 [SMILES: CC(C)(C)OC(=O)Nc1sc2cncc(Br)c2c1C#N]

To a solution of 2-amino-4-bromothieno[2,3-c]pyridine-3-carbonitrile (1.8 g, 7.08 mmol) in THF (35.4 mL) were added DMAP (35 mg, 0.283 mmol), DIPEA (1.49 mL, 8.50 mmol) and Boc₂O (1.55 g, 7.08 mmol) and the reaction mixture was stirred at r.t. for 1 h before heating to 40° C. for 2 h. The mixture was diluted with water and filtered. The filtrate was washed with water and air dried to provide the title compound, which was used in the next step without further purification. LCMS calc. for C₁₃H₁₃BrN₃O₂S (M+H)⁺ m/z=354.0, 356.0; found 354.0, 356.0.

### Step 6. tert-Butyl (4-bromo-3-cyanothieno[2,3-c]pyridin-2-yl)((2-(trimethylsilyl)ethoxy)methyl)carbamate

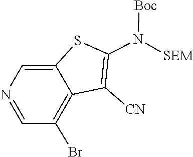 [SMILES: CC(C)(C)OC(=O)N(COCC[Si](C)(C)C)c1sc2cncc(Br)c2c1C#N]

To a solution of tert-butyl (4-bromo-3-cyanothieno[2,3-c]pyridin-2-yl)carbamate (2.0 g, 6.21 mmol) in DMF (28.2 mL) at 0° C. was added NaH (60% w/w mineral oil, 248 mg, 6.21 mmol) and the mixture was stirred at r.t. for 30 min., then re-cooled to 0° C. SEMCl (90% wt, 1.33 mL, 6.78 mmol) was added and the reaction mixture was warmed to r.t., then quenched with sat. NaHCO₃. The mixture was extracted with EtOAc. The organic layer was washed with water and brine, dried over Na₂SO₄ and concentrated. The crude product was purified by FCC (0-70% EtOAc in hexanes) to provide the title compound (950 mg, 35%). LCMS calc. for C₁₉H₂₇BrN₃O₃SSi (M+H)⁺ m/z=484.1, 486.1; found 484.1, 486.1.

### Step 7. 2-Amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile

A solution of (3R,5S)-3-(7-chloro-9-fluoro-2-methyl-8-(tributylstannyl)-5,6-dihydro-4H-benzo[de][1,6]naphthyridin-4-yl)-1-(methyl-d₃)-5-(trifluoromethyl)pyrrolidin-2-one (Intermediate 14, 63 mg, 0.091 mmol), tert-butyl (4-bromo-3-cyanothieno[2,3-c]pyridin-2-yl)((2 (trimethylsilyl)ethoxy)methyl)carbamate (88 mg, 0.182 mmol), Pd(PPh₃)₄ (21.0 mg, 0.018 mmol), CuI (3.5 mg, 0.018 mmol), and LiCl (11.6 mg, 0.272 mmol) in 1,4-dioxane (908 μL) was sparged with N₂ and then heated to 110° C. for 2 h. Upon completion, the reaction mixture was diluted with EtOAc, washed with brine, dried over Na₂SO₄, concentrated and the crude was purified by FCC (0-10% MeOH in DCM). The product was dissolved in TFA/DCM, the resulting mixture was warmed to 40° C. After stirring for 30 min. the reaction mixture was concentrated, diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 88a. Diastereomer 1. Peak 1. LCMS calc. for C₂₆H₁₇D₃ClF₄N₆OS (M+H)⁺ m/z=578.1; found 578.1. This is the potent peak.

Example 88b. Diastereomer 2. Peak 2. LCMS calc. for for C₂₆H₁₇D₃ClF₄N₆OS (M+H)⁺ m/z=578.1; found 578.1.

The compounds are understood to comprise atropisomers, i.e., (Rₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile; and (Sₐ)-2-amino-4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile.

The alternative isomers, i.e., (Rₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile; and (Sₐ)-2-amino-4-(7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile; are prepared by an analogous procedure using the alternative diastereomer of Intermediate 11 as starting material.

### Example 89. 8-(2-Amino-3-cyano-7-fluorobenzo[b]thiophen-4-yl)-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile

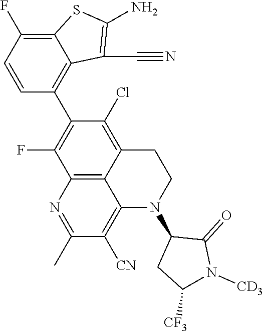 [SMILES: [2H]C([2H])([2H])N1C(=O)[C@H](N2CCc3c(Cl)c(-c4ccc(F)c5sc(N)c(C#N)c45)c(F)c4nc(C)c(C#N)c2c34)C[C@H]1C(F)(F)F]

### Step 1. tert-Butyl (4-(3-bromo-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate

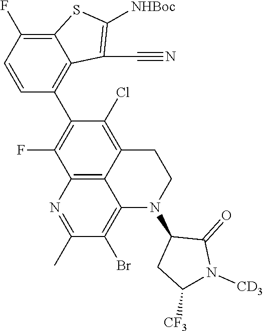 [SMILES: [2H]C([2H])([2H])N1C(=O)[C@H](N2CCc3c(Cl)c(-c4ccc(F)c5sc(NC(=O)OC(C)(C)C)c(C#N)c45)c(F)c4nc(C)c(Br)c2c34)C[C@H]1C(F)(F)F]

To a 40 mL vial, were added tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (Ex. 41, 750 mg, 1.08 mmol), MeCN (10 mL), HFIP (2.0 mL) and 1-bromopyrrolidine-2,5-dione (192 mg, 1.08 mmol). The resulting mixture was stirred at 0° C. for 40 min. before being quenched with aq. Na₂S₂O₃/NaHCO₃, diluted with EtOAc, and the organic phase was dried over Na₂SO₄ and concentrated. The crude product was purified by prep. SFC (7-27% MeOH in CO₂, 2-PIC column, 140 mL/min. flow rate) to provide the title compound. LCMS calc. for C₃₂H₂₄D₃BrClF₅N₅O₃S (M+H)⁺ m/z=773.1, 775.1; found 773.1, 775.1.

### Step 2. 8-(2-Amino-3-cyano-7-fluorobenzo[b]thiophen-4-yl)-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile

To a mixture of tert-butyl (4-(3-bromo-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (23 mg, 0.030 mmol), zinc cyanide (3.5 mg, 0.030 mmol), Pd(PPh₃)₄ (3.4 mg, 3.0 μmol) was added DMF (297 μL) and the reaction flask was evacuated, back filled with N₂, then stirred at 100° C. for 3 h. The reaction was quenched with water and 30%. aq. NH₄OH, then extracted with EtOAc. The organic layer was washed with water and brine, dried over Na₂SO₄ and concentrated. The crude product was treated with TFA (1 mL) at rt for 1 h. The volatile was removed, and the residue was diluted with MeCN and purified by prep. HPLC (XBRIDGE® Cl₈ column, eluting with a gradient of MeCN/water containing 0.1% NH₄OH, at flow rate of 60 mL/min.) to afford the title compound. LCMS calc. for C₂₈H₁₆D₃ClF₅N₆OS (M+H)⁺ m/z=620.1; found 620.1.

The compound is understood to comprise atropisomers, i.e., (Rₐ)-8-(2-amino-3-cyano-7-fluorobenzo[b]thiophen-4-yl)-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile; and (Sₐ)-8-(2-amino-3-cyano-7-fluorobenzo[b]thiophen-4-yl)-7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile.

The alternative isomers, i.e.: (Rₐ)-8-(2-amino-3-cyano-7-fluorobenzo[b]thiophen-4-yl)-7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridine-3-carbonitrile; and (Sₐ)-8-(2-amino-3-cyano-7-fluorobenzo[b]thiophen-4-yl)-7-chloro-9-fluoro-2-methyl-4-((3S,5R)-1-(methyl-d₃)-2-oxo-5-(trifluoromethyl)pyrrolidin-3-yl)-5,6-dihydro-4H-benzo[de][1,6]naphthyridine-3-carbonitrile; are prepared by an analogous procedure using the alternative diastereomer of Intermediate 11 as starting material.

### Example 90. 8-(2-Amino-3-cyano-7-fluorobenzo[b]thiophen-4-yl)-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile

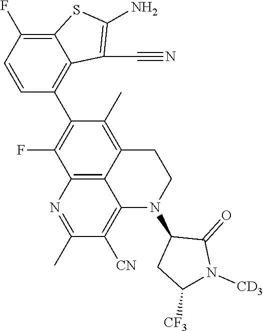 [SMILES: [2H]C([2H])([2H])N1C(=O)[C@H](N2CCc3c(C)c(-c4ccc(F)c5sc(N)c(C#N)c45)c(F)c4nc(C)c(C#N)c2c34)C[C@H]1C(F)(F)F]

### Step 1. tert-Butyl (3-cyano-7-fluoro-4-(9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophen-2-yl)carbamate

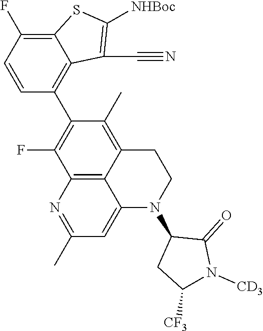 [SMILES: [2H]C([2H])([2H])N1C(=O)[C@H](N2CCc3c(C)c(-c4ccc(F)c5sc(NC(=O)OC(C)(C)C)c(C#N)c45)c(F)c4nc(C)cc2c34)C[C@H]1C(F)(F)F]

To a 1 dram vial, was added K3PO₄ (418 mg, 1.97 mmol), methanesulfonato(2-dicyclohexylphosphino-2′,6′-dimethoxy-1,1′-biphenyl)(2′-methylamino-1,1′-biphenyl-2-yl)palladium(II) dichloromethane adduct (117 mg, 0.148 mmol), methylboronic acid (295 mg, 4.92 mmol), and tert-butyl (4-(7-chloro-9-fluoro-2-methyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (Ex. 41, 342 mg, 0.492 mmol), and the vial was evacuated, back filled with N₂, before 1,4-dioxane (2050 μL) and water (410 μL) were added. The resulting mixture was heated to 95° C. for 4 h before cooling to r.t. The mixture was diluted with EtOAc, quenched with sat. aq. NaHCO₃, separated, and the organic phase was dried over Na₂SO₄ and concentrated. The crude product was purified by FCC (0-100% EtOAc in hexanes) to provide the title compound (128 mg. 39%). LCMS calc. for C₃₃H₂₈D₃F₅N₅O₃S (M+H)⁺ m/z=675.2; found 675.2.

### Step 2. tert-Butyl (4-(3-bromo-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate

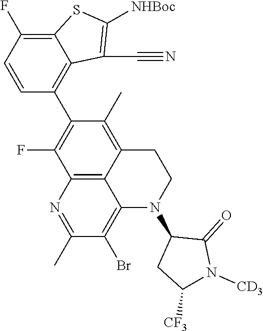 [SMILES: [2H]C([2H])([2H])N1C(=O)[C@H](N2CCc3c(C)c(-c4ccc(F)c5sc(NC(=O)OC(C)(C)C)c(C#N)c45)c(F)c4nc(C)c(Br)c2c34)C[C@H]1C(F)(F)F]

To a 0° C. solution of tert-butyl (3-cyano-7-fluoro-4-(9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)benzo[b]thiophen-2-yl)carbamate (75.3 mg, 0.112 mmol) in DCM (1116 μL), was added 1-bromopyrrolidine-2,5-dione (19.9 mg, 0.112 mmol) at 0° C., the result mixture was stirred at 0° C. for 20 min. before diluted with EtOAc and quenched with aq. NaHCO₃/Na₂S₂O₃, phased separated, the organic phase was dried over Na₂SO₄, and concentrated. The crude product was purified by FCC (0-100% EtOAc in hexanes) to provide the title compound (38.4 mg. 46%). LCMS calc. for C₃₃H₂₇D₃BrF₅N₅O₃S (M+H)⁺ m/z=753.1, 755.1; found 753.1, 755.1.

### Step 3. 8-(2-Amino-3-cyano-7-fluorobenzo[b]thiophen-4-yl)-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile

To a mixture of tert-butyl (4-(3-bromo-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-3-cyano-7-fluorobenzo[b]thiophen-2-yl)carbamate (38.4 mg, 0.051 mmol), zinc cyanide (6.0 mg, 0.051 mmol), Pd(PPh₃)₄ (11.8 mg, 10 μmol) was added DMF (510 μL) and the reaction flask was evacuated, back filled with N₂, then stirred at 100° C. for 2 h. The reaction was quenched with water and sat. aq. NH₄OH, then extracted with EtOAc. The organic layer was washed with water and brine, dried over Na₂SO₄ and concentrated. The crude product purified by FCC (0-100% EtOAc in hexanes) to provide the intermediate. The intermediate 25 was dissolved in dissolved in TFA/DCM (1:1) (1.0 mL), the resulting mixture stirred for 20 min. The volatile was removed, and the residue was diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₂₉H₁₉D₃F₅N₆OS (M+H)⁺ m/z=600.2; found 600.2.

The compound is understood to comprise atropisomers, i.e., (Rₐ)-8-(2-amino-3-cyano-7-fluorobenzo[b]thiophen-4-yl)-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile; and (Sₐ)-8-(2-amino-3-cyano-7-fluorobenzo[b]thiophen-4-yl)-9-fluoro-2,7-dimethyl-4-((3R,5S)-1-(methyl-d₃)-2-oxo-5-(trifluoromethyl)pyrrolidin-3-yl)-5,6-dihydro-4H-benzo[de][1,6]naphthyridine-3-carbonitrile.

The alternative isomers, i.e., (Rₐ)-8-(2-amino-3-cyano-7-fluorobenzo[b]thiophen-4-yl)-9-fluoro-2,7-dimethyl-4-((3S,5R)-1-(methyl-d₃)-2-oxo-5-(trifluoromethyl)pyrrolidin-3-yl)-5,6-dihydro-4H-benzo[de][1,6]naphthyridine-3-carbonitrile; and (Sₐ)-8-(2-amino-3-cyano-7-fluorobenzo[b]thiophen-4-yl)-9-fluoro-2,7-dimethyl-4-((3S,5R)-1-(methyl-d₃)-2-oxo-5-(trifluoromethyl)pyrrolidin-3-yl)-5,6-dihydro-4H-benzo[de][1,6]naphthyridine-3-carbonitrile; are prepared by an analogous procedure using the alternative diastereomer of Intermediate 11 as starting material.

### Example 91a and 91b. 2-Amino-4-(2-(azetidin-1-ylmethyl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

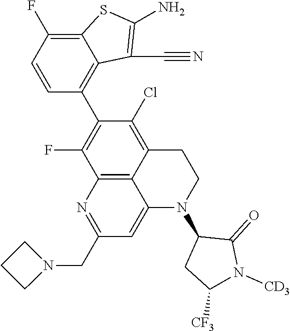 [SMILES: [2H]C([2H])([2H])N1C(=O)[C@H](N2CCc3c(Cl)c(-c4ccc(F)c5sc(N)c(C#N)c45)c(F)c4nc(CN5CCC5)cc2c34)C[C@H]1C(F)(F)F]

### Step 1. (3R,5S)-3-(2-(Azetidin-1-ylmethyl)-8-bromo-7-chloro-9-fluoro-5,6-dihydro-4H-benzo[de][1,6]naphthyridin-4-yl)-1-(methyl-d₃)-5-(trifluoromethyl)pyrrolidin-2-one

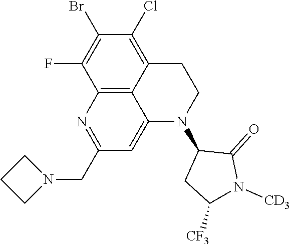 [SMILES: [2H]C([2H])([2H])N1C(=O)[C@H](N2CCc3c(Cl)c(Br)c(F)c4nc(CN5CCC5)cc2c34)C[C@H]1C(F)(F)F]

To the mixture of azetidine HCl (32.7 mg, 0.350 mmol) in THF (499 μL), DCE (499 μL), and HFIP (166 μL) was added DIPEA (81 μL, 0.47 mmol). The resulting mixture was stirred at r.t. followed by the addition of (8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridine-2-carbaldehyde (Intermediate 15, 58.0 mg, 0.117 mmol), NaBH(OAc)₃ (123 mg, 0.583 mmol) and AcOH (67 μL, 1.17 mmol). The resulting mixture was stirred at r.t. for another 10 min. before being diluted with EtOAc. The aqueous layer was extracted with additional EtOAc and the combined organics were washed with brine, dried over Na₂SO₄ and concentrated. The crude product was used in the next step without further purification. LCMS calc. for C₂₁H₁₃D₃BrClF₄N₄O (M+H)⁺ m/z=538.1, 540.1; found 538.1, 540.1.

### Step 2. 2-Amino-4-(2-(azetidin-1-ylmethyl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

A solution of (3R,5S)-3-(2-(azetidin-1-ylmethyl)-8-bromo-7-chloro-9-fluoro-5,6-dihydro-4H-benzo[de][1,6]naphthyridin-4-yl)-1-(methyl-d₃)-5-(trifluoromethyl)pyrrolidin-2-one (60 mg, 0.11 mmol), tert-butyl (3-cyano-4-(5,5-dimethyl-1,3,2-dioxaborinan-2-yl)-7-fluorobenzo[b]thiophen-2-yl)carbamate (113 mg, 0.278 mmol), Pd-117 (19.9 mg, 0.028 mmol), and K₂CO₃ (61.6 mg, 0.445 mmol) in 1,4-dioxane (1114 μL) was sparged with N₂ and heated to 100° C. for 30 min. The mixture was cooled to r.t, diluted with EtOAc, filtered, washed with brine, dried over Na₂SO₄, filtered and concentrated. The crude product was dissolved in 1:1 DCM/TFA. After stirring at r.t. for 30 min., the mixture was concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 91a. Diastereomer 1. Peak 1. LCMS calc. for C₃₀H₂₂D₃ClF₆N₆OS (M+H)⁺ m/z=650.2; found 650.2. This is the potent peak.

Example 91b. Diastereomer 2. Peak 2. LCMS calc. for C₃₀H₂₂D₃ClF₆N₆OS (M+H)⁺ m/z=650.2; found 650.2.

The compound is understood to comprise atropisomers, i.e., (Rₐ)-2-amino-4-(2-(azetidin-1-ylmethyl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(azetidin-1-ylmethyl)-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(2-(azetidin-1-ylmethyl)-7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(2-(azetidin-1-ylmethyl)-7-chloro-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 92. 2-Amino-4-(7-chloro-9-fluoro-4-((3R,5S)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile

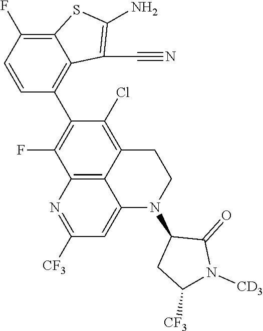 [SMILES: [2H]C([2H])([2H])N1C(=O)[C@H](N2CCc3c(Cl)c(-c4ccc(F)c5sc(N)c(C#N)c45)c(F)c4nc(C(F)(F)F)cc2c34)C[C@H]1C(F)(F)F]

### Step 1. (3R,5S)-3-(8-Bromo-7-chloro-9-fluoro-2-(trifluoromethyl)-5,6-dihydro-4H-benzo[de][1,6]naphthyridin-4-yl)-1-(methyl-d₃)-5-(trifluoromethyl)pyrrolidin-2-one

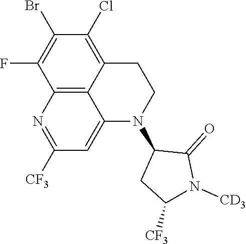 [SMILES: [2H]C([2H])([2H])N1C(=O)[C@H](N2CCc3c(Cl)c(Br)c(F)c4nc(C(F)(F)F)cc2c34)C[C@H]1C(F)(F)F]

To a 1 dram vial was added CuI (19.2 mg, 0.101 mmol), (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 200 mg, 0.336 mmol), the vial was evacuated and back filed with N₂, followed by NMP (1.7 mL), and methyl fluorosulfonyldifluoroacetate (106 μL, 0.841 mmol). The resulting mixture was heated to 80° C. for 6 h before cooling to r.t. The reaction was diluted with EtOAc, quenched with sat. aq. NaHCO₃, separated, the organic phase was dried over Na₂SO₄, and concentrated. The crude product was purified by FCC (10-70% EtOAc in hexanes) to provide the title compound (159.5 mg, 88%).

### Step 2. 2-Amino-4-(7-chloro-9-fluoro-4-((3R,5S)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile

To the mixture of K₃PO₄ (126 mg, 0.593 mmol), tert-butyl (3-cyano-4-(5,5-dimethyl-1,3,2-dioxaborinan-2-yl)-7-fluorobenzo[b]thiophen-2-yl)carbamate (240 mg, 0.593 mmol), (3R,5S)-3-(8-bromo-7-chloro-9-fluoro-2-(trifluoromethyl)-5,6-dihydro-4H-benzo[de][1,6]naphthyridin-4-yl)-1-(methyl-d₃)-5-(trifluoromethyl)pyrrolidin-2-one (159 mg, 0.297 mmol), (2R,3R)-BaryPhos (24.9 mg, 47 μmol), and Pd₂(dba)₃ (23.9 mg, 42 μmol) was added DCE (3.0 mL) under N₂. The mixture was warmed to 50° C. for 4 h. The mixture was cooled to r.t., diluted with EtOAc and filtered. The filtrate was concentrated, and the residue was purified by FCC (10-70% EtOAc in hexanes). The intermediate was dissolved in 1:1 TFA/DCM (1 mL) and the reaction mixture was stirred at r.t. for 30 mins, then diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₂₇H₁₄D₃ClF₈N₅OS (M+H)⁺ m/z=649.1; found 649.1.

The compounds are understood to comprise atropisomers, i.e., (Rₐ)-2-amino-4-(7-chloro-9-fluoro-4-((3R,5S)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile; are prepared by an analogous procedure using the alternative diastereomer of Intermediate 11 as starting material.

### Example 93a, 93b, 93c, and 93d. 2-Amino-4-(7-chloro-9-fluoro-2-methyl-4-(1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile

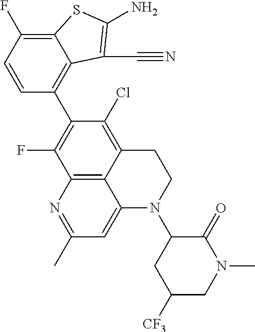 [SMILES: Cc1cc2c3c(c(Cl)c(-c4ccc(F)c5sc(N)c(C#N)c45)c(F)c3n1)CCN2C1CC(C(F)(F)F)CN(C)C1=O]

### Step 1. 1-Methyl-5-(trifluoromethyl)piperidin-2-one

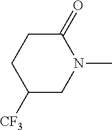

To a mixture of 5-(trifluoromethyl)piperidin-2-one (1.0 g, 5.98 mmol) and Cs₂CO₃ (3.90 g, 12.0 mmol) in MeCN (29.9 mL), was added iodomethane-d₃ (0.748 mL, 12.0 mmol) at r.t., and the reaction mixture was heated to 70° C. overnight. The mixture was diluted with DCM, filtered through a plug of diatomaceous earth, and concentrated. The crude product was purified by FCC (0-50% EtOAc in hexanes) to provide the title compound (750 mg, 69%). LCMS calc. for C₇H₁₁F₃NO (M+H)⁺ m/z=182.1; found 182.1.

### Step 2. 3-Amino-1-methyl-5-(trifluoromethyl)piperidin-2-one

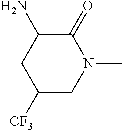

To a solution of 1-methyl-5-(trifluoromethyl)piperidin-2-one (200 mg, 1.10 mmol) in THF (5.5 mL) at −78° C. was added LDA (1.0 M in THF/hexanes, 1.66 mL, 1.66 mmol) and the mixture was stirred at −78° C. for 35 min. A solution of 2,4,6-triisopropylbenzenesulfonyl azide (512.4 mg, 1.67 mmol) in toluene (5.9 mL) was then added dropwise while maintaining the reaction temperature at −78° C. The reaction mixture was then slowly warmed to r.t. over 4 h. The reaction was quenched with AcOH (0.13 mL, 2.2 mmol), the mixture was diluted with 30% aq. NH₄Cl and extracted with EtOAc. The organic layer was washed with brine, dried over Na₂SO₄ and concentrated. The residue was suspended in EtOAc (2.0 mL) and Pd/C (10% by weight, 29.4 mg, 0.276 mmol) and the reaction flask was bubbled with H₂ and stirred under H₂ for 12 h. The mixture was filtered through a plug of diatomaceous earth and the filtrate was concentrated. The crude product was used in the next step without further purification. LCMS calc. for C₇H₁₂F₃N₂O (M+H)⁺ m/z=197.1; found 197.1.

### Step 3. 3-(8-Bromo-7-chloro-9-fluoro-2-methyl-5,6-dihydro-4H-benzo[de][1,6]naphthyridin-4-yl)-1-methyl-5-(trifluoromethyl)piperidin-2-one

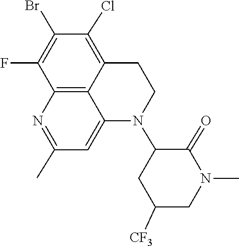 [SMILES: Cc1cc2c3c(c(Cl)c(Br)c(F)c3n1)CCN2C1CC(C(F)(F)F)CN(C)C1=O]

The mixture of 2-(7-bromo-6-chloro-8-fluoro-2-methyl-4-(methylthio)quinolin-5-yl)acetaldehyde (Intermediate 8, 200 mg, 0.552 mmol) and 3-amino-1-methyl-5-(trifluoromethyl)piperidin-2-one (108 mg, 0.552 mmol) in THF (5.5 mL) and MeOH (5.5 mL) was stirred at r.t. for 1 h before NaBH₃CN (34.7 mg, 0.552 mmol) was added. The resulting mixture was stirred at r.t. for another 1 h then concentrated. The residue was diluted with MeOH, filtered and purified by prep. HPLC (XBRIDGE® C₁₈ column, eluting with a gradient of MeCN/water containing 0.1% NH₄OH, at flow rate of 60 mL/min.) to afford the title compound (75 mg, 28%). LCMS calc. for C₁₉H₁₃BrClF₄N₃O (M+H)⁺ m/z=494.0, 496.0; found 494.0, 496.0.

### Step 4. 2-Amino-4-(7-chloro-9-fluoro-2-methyl-4-(1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile

To the mixture of K₃PO₄ (64.4 mg, 0.303 mmol), tert-butyl (3-cyano-4-(5,5-dimethyl-1,3,2-dioxaborinan-2-yl)-7-fluorobenzo[b]thiophen-2-yl)carbamate (123 mg, 0.303 mmol), 3-(8-bromo-7-chloro-9-fluoro-2-methyl-5,6-dihydro-4H-benzo[de][1,6]naphthyridin-4-yl)-1-methyl-5-(trifluoromethyl)piperidin-2-one (75 mg, 0.152 mmol)), (2R,3R)-BaryPhos (11.1 mg, 0.021 mmol), and Pd(dba)₂ (10.5 mg, 0.018 mmol) was added DCE (1516 μl) under N₂ at r.t. The mixture was stirred at r.t. for 5 min. before heated to 50° C. for 12 h. The mixture was diluted with EtOAc, filtered, concentrated and purified by FCC (10-100% EtOAc in hexanes) to give the product. The product was dissolved in 1:1 MeCN/TFA (4 mL), then the mixture was stirred at r.t. for 1 h, concentrated, diluted with MeCN and purified by prep. HPLC (XBRIDGE® C₁₈ column, eluting with a gradient of MeCN/water containing 0.1% NH₄OH, at flow rate of 60 mL/min.) to afford the title compound as four peaks.

Example 93a. Diastereomer 1. Peak 1. LCMS calc. for C₂₈H₂₂ClF₅N₅OS (M+H)⁺ m/z=606.1; found 606.1.

Example 93b. Diastereomer 2. Peak 2. LCMS calc. for C₂₈H₂₂ClF₅N₅OS (M+H)⁺ m/z=606.1; found 606.1. This is the potent peak.

Example 93c. Diastereomer 3. Peak 3. LCMS calc. for C₂₈H₂₂ClF₅N₅OS (M+H)⁺ m/z=606.1; found 606.1.

Example 93d. Diastereomer 4. Peak 4. LCMS calc. for C₂₈H₂₂ClF₅N₅OS (M+H)⁺ m/z=606.1; found 606.1.

The compounds are understood to comprise atropisomers and/or diastereoisomers, i.e., (Rₐ)-2-amino-4-(7-chloro-9-fluoro-2-methyl-4-((3R,5R)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3R,5R)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3S,5R)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3S,5R)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3R,5S)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3R,5S)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3S,5S)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((3S,5S)-1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile.

### Example 94a and 94b. 2-Amino-4-(7-chloro-2-((3S,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

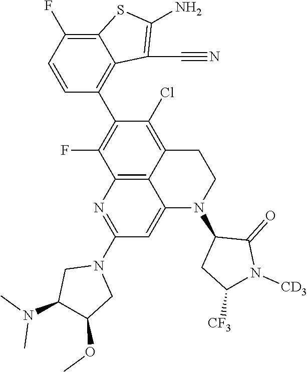 [SMILES: [2H]C([2H])([2H])N1C(=O)[C@H](N2CCc3c(Cl)c(-c4ccc(F)c5sc(N)c(C#N)c45)c(F)c4nc(N5C[C@@H](OC)[C@@H](N(C)C)C5)cc2c34)C[C@H]1C(F)(F)F]

### Step 1. (3R,5S)-3-(2-((3S,4R)-3-Amino-4-methoxypyrrolidin-1-yl)-8-bromo-7-chloro-9-fluoro-5,6-dihydro-4H-benzo[de][1,6]naphthyridin-4-yl)-1-(methyl-d₃)-5-(trifluoromethyl)pyrrolidin-2-one

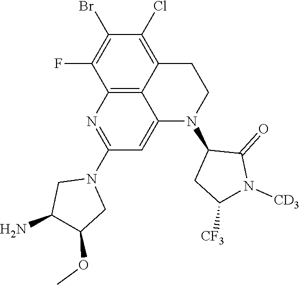 [SMILES: [2H]C([2H])([2H])N1C(=O)[C@H](N2CCc3c(Cl)c(Br)c(F)c4nc(N5C[C@H](N)[C@H](OC)C5)cc2c34)C[C@H]1C(F)(F)F]

In a 1 dram vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 180 mg, 0.302 mmol), tert-butyl ((3S,4R)-4-methoxypyrrolidin-3-yl)carbamate (55 mg, 0.32 mmol), L-proline (6.1 mg, 0.053 mmol), CuI (5.8 mg, 0.030 mmol) and K₂CO₃ (95 mg, 0.69 mmol) were dissolved in DMSO (1007 μL) under N₂. The reaction mixture was heated to 80° C. until full conversion indicated by LCMS. The reaction mixture was diluted with EtOAc and 30% aq. NH₄OH, then extracted with EtOAc. The organic layers were washed with aq. NH₄OH once followed by 10% aq. LiCl, dried over MgSO₄ and concentrated. The crude product dissolved in TFA (3.0 mL) and was stirred at r.t. for 30 min. before being concentrated. The product was diluted with DCM, washed with sat. aq. NaHCO₃, extracted with DCM, the combined organic phases were dried over MgSO₄ and concentrated. The crude product was used in the next step without further purification. LCMS calc. for C₂₂H₂₁D₃BrClF₄N₅O₂ (M+H)⁺ m/z=583.1, 585.1; found 583.1, 585.1.

### Step 2. (3R,5S)-3-(8-Bromo-7-chloro-2-((3S,4R)-3-(dimethylamino)-4-methoxypyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one

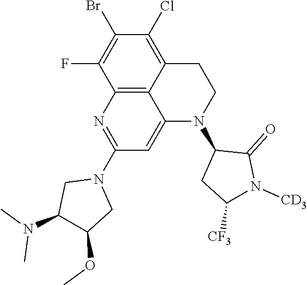 [SMILES: [2H]C([2H])([2H])N1C(=O)[C@H](N2CCc3c(Cl)c(Br)c(F)c4nc(N5C[C@@H](OC)[C@@H](N(C)C)C5)cc2c34)C[C@H]1C(F)(F)F]

In a 2 dram vial, (3R,5S)-3-(2-((3S,4R)-3-amino-4-methoxypyrrolidin-1-yl)-8-bromo-7-chloro-9-fluoro-5,6-dihydro-4H-benzo[de][1,6]naphthyridin-4-yl)-1-(methyl-d₃)-5-(trifluoromethyl)pyrrolidin-2-one (167 mg, 0.286 mmol), formaldehyde (596 μL, 8.01 mmol), and formic acid (598 μL, 15.6 mmol) were mixed and heated to 100° C. for 1 h. The reaction mixture was cooled to 0° C. and quenched with 30% aq. NH₄OH. The mixture was extracted with DCM, dried over MgSO₄, filtered, and volatiles were removed. The crude residue was purified by SFC (6-16% MeOH in CO₂, 2-PIC column, 140 mL/min. flow rate) to provide the title compound. LCMS calc. for C₂₄H₂₅D₃BrClF₄N₅O₂ (M+H)⁺ m/z=611.1, 613.1; found 611.1, 613.1.

### Step 3. 2-Amino-4-(7-chloro-2-((3S,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

In a 1 dram vial, (3R,5S)-3-(8-bromo-7-chloro-2-((3S,4R)-3-(dimethylamino)-4-methoxypyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one (36.5 mg, 0.060 mmol), tert-butyl (3-cyano-4-(5,5-dimethyl-1,3,2-dioxaborinan-2-yl)-7-fluorobenzo[b]thiophen-2-yl)carbamate (48.2 mg, 0.119 mmol), dichloro[bis(diphenylphosphinophenyl)ether]palladium(II) (12.8 mg, 0.018 mmol), and K₂CO₃ (24.7 mg, 0.179 mmol) were dissolved in 1,4-dioxane (298 μL) under N₂ and heated to 100° C. for 1 h. The mixture was cooled to r.t., diluted with water, extracted with DCM, dried over MgSO₄, filtered, and concentrated. The crude product was dissolved in TFA (1 mL) and stirred at r.t. for 30 min. before being concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 94a. Diastereomer 1. Peak 1. LCMS calc. for C₃₃H₂₉D₃ClF₅N₇O₂S (M+H)⁺ m/z=723.2; found 723.2. This is the potent peak.

Example 94b. Diastereomer 2. Peak 2. LCMS calc. for C₃₃H₂₉D₃ClF₅N₇O₂S (M+H)⁺ m/z=723.2; found 723.2.

The compounds are understood to comprise atropisomers and/or diasteroisomers, i.e., (Rₐ)-2-amino-4-(7-chloro-2-((3S,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3S,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Rₐ)-2-amino-4-(7-chloro-2-((3R,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3R,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((3S,4R)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((3S,4R)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((3R,4S)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3R,4S)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the (3S,5R)-diastereomer of Intermediate 11 as starting material.

### Example 95. 2-Amino-4-(7-chloro-2-(((1,1-dioxidothietan-3-yl)oxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

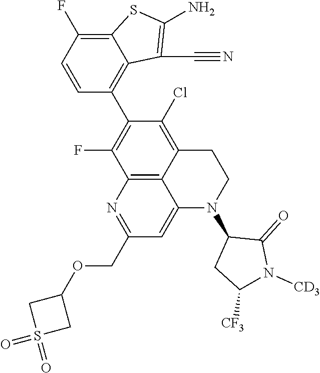 [SMILES: [2H]C([2H])([2H])N1C(=O)[C@H](N2CCc3c(Cl)c(-c4ccc(F)c5sc(N)c(C#N)c45)c(F)c4nc(COC5CS(=O)(=O)C5)cc2c34)C[C@H]1C(F)(F)F]

### Step 1. (3R,5S)-3-(8-Bromo-7-chloro-2-(((1,1-dioxidothietan-3-yl)oxy)methyl)-9-fluoro-5,6-dihydro-4H-benzo[de][1,6]naphthyridin-4-yl)-1-(methyl-d₃)-5-(trifluoromethyl)pyrrolidin-2-one

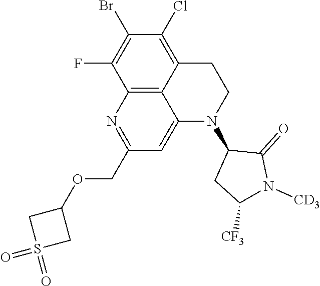 [SMILES: [2H]C([2H])([2H])N1C(=O)[C@H](N2CCc3c(Cl)c(Br)c(F)c4nc(COC5CS(=O)(=O)C5)cc2c34)C[C@H]1C(F)(F)F]

To the mixture of (3R,5S)-3-(8-bromo-7-chloro-9-fluoro-2-(hydroxymethyl)-5,6-dihydro-4H-benzo[de][1,6]naphthyridin-4-yl)-1-(methyl-d₃)-5-(trifluoromethyl)pyrrolidin-2-one (Intermediate 15, 21.3 mg, 0.043 mmol), 3-bromothietane 1,1-dioxide (15.8 mg, 0.085 mmol), and NaH (60% weight in mineral oil, 3.4 mg, 0.085 mmol) was added THF (426 μL) under N₂ at r.t. The resulting mixture was stirred at r.t. for 15 h before quenched with sat. aq. NaHCO₃, diluted with EtOAc, phases separated, the organic phase was washed with brine, dried over Na₂SO₄, filtered, and concentrated. The crude product was purified by FCC (10-85% EtOAc in hexanes) to provide the title compound (15.6 mg, 61%). LCMS calc. for C₂₁H₁₇D₃BrClF₄N₃O₄S (M+H)⁺ m/z=603.0, 605.0; found 603.0, 605.0.

### Step 2. 2-Amino-4-(7-chloro-2-(((1,1-dioxidothietan-3-yl)oxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (13.7 mg, 0.065 mmol), tert-butyl (3-cyano-4-(5,5-dimethyl-1,3,2-dioxaborinan-2-yl)-7-fluorobenzo[b]thiophen-2-yl)carbamate (26.1 mg, 0.065 mmol), (3R,5S)-3-(8-bromo-7-chloro-2-(((1,1-dioxidothietan-3-yl)oxy)methyl)-9-fluoro-5,6-dihydro-4H-benzo[de][1,6]naphthyridin-4-yl)-1-(methyl-d₃)-5-(trifluoromethyl)pyrrolidin-2-one (15.6 mg, 0.026 mmol), (2R,3R)-BaryPhos (2.7 mg, 5.2 μmol), and Pd(dba)₂ (2.7 mg, 4.7 μmol) was added DCE (258 μL) under N₂. The mixture was warmed to 50° C. for 4 h. The mixture was cooled to r.t., diluted with EtOAc and filtered. The filtrate was concentrated, and the residue was purified by FCC (10-100% EtOAc in hexanes). The intermediate was dissolved in TFA (1 mL) and the mixture was stirred at r.t. for 10 min. before being concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₃₀H₂₁D₃ClF₅N₅O₄S₂(M+H)⁺ m/z=715.1; found 715.1. This is the potent peak.

The compounds are understood to comprise atropisomers, i.e., (Rₐ)-2-amino-4-(7-chloro-2-(((1,1-dioxidothietan-3-yl)oxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(((1,1-dioxidothietan-3-yl)oxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-(((1,1-dioxidothietan-3-yl)oxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-(((1,1-dioxidothietan-3-yl)oxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using (3S,5R)-diastereomer of Intermediate 11 as starting material.

### Examples 96-97

Compounds in Table 12 were prepared according to the general procedure described for Example 95 using the appropriate starting material.

TABLE 12

| # | Structure | Name | [M + H] |
|---|---|---|---|
| 96 | 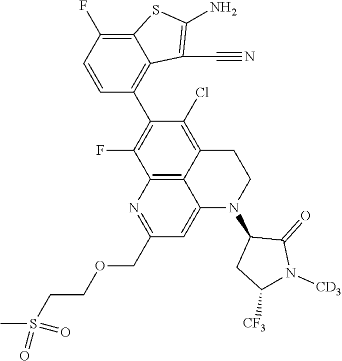 [SMILES: [2H]C([2H])([2H])N1C(=O)[C@H](N2CCc3c(Cl)c(-c4ccc(F)c5sc(N)c(C#N)c45)c(F)c4nc(COCCS(C)(=O)=O)cc2c34)C[C@H]1C(F)(F)F] | 2-amino-4-(7-chloro-9-fluoro-4- ((3R,5S)-1-(methyl-d₃)-2-oxo-5- (trifluoromethyl)pyrrolidin-3-yl)-2-((2- (methylsulfonyl)ethoxy)methyl)-5,6- dihydro-4H- benzo[de][1,6]naphthyridin-8-yl)-7- fluorobenzo[b]thiophene-3- carbonitrile | 717.1 |
| 97 | 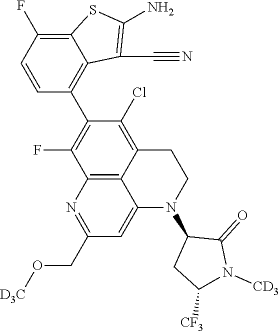 [SMILES: [2H]C([2H])([2H])OCc1cc2c3c(c(Cl)c(-c4ccc(F)c5sc(N)c(C#N)c45)c(F)c3n1)CCN2[C@@H]1C[C@@H](C(F)(F)F)N(C([2H])([2H])[2H])C1=O] | 2-amino-4-(7-chloro-9-fluoro-2- ((methoxy-d3)methyl)-4-((3R,5S)-1- (methyl-d₃)-2-oxo-5- (trifluoromethyl)pyrrolidin-3-yl)-5,6- dihydro-4H- benzo[de][1,6]naphthyridin-8-yl)-7- fluorobenzo[b]thiophene-3- carbonitrile | 628.1 |

The compounds of Table 12 are understood to comprise mixtures of diastereoisomers and/or atropisomers.

### Example 96

The compounds of Example 96 are understood to comprise atropisomers, i.e., (Rₐ)-2-amino-4-(7-chloro-9-fluoro-4-((3R,5S)-1-(methyl-d₃)-2-oxo-5-(trifluoromethyl) pyrrolidin-3-yl)-2-((2-(methyl sulfonyl)ethoxy)methyl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3R,5S)-1-(methyl-d₃)-2-oxo-5-(trifluoromethyl) pyrrolidin-3-yl)-2-((2-(methyl sulfonyl)ethoxy)methyl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-4-((3S,5R)-1-(methyl-d₃)-2-oxo-5-(trifluoromethyl) pyrrolidin-3-yl)-2-((2-(methylsuIfonyl)ethoxy) methyl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((3S,5R)-1-(methyl-d₃)-2-oxo-5-(trifluoromethyl) pyrrolidin-3-yl)-2-((2-(methylsulfonyl)ethoxy)methyl)-5,6-dihydro-4H-benzo[de][1,6]naphthyridin-8-yl)-7-fluorobenzo[b]thiophene-3-carbonitrile; are prepared by an analogous procedure using the (3S,5R)-diastereomer of Intermediate 11 as starting material.

### Example 97

The compounds of Example 97 are understood to comprise atropisomers, i.e., (Rₐ)-2-amino-4-(7-chloro-9-fluoro-2-((methoxy-d3)methyl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-((methoxy-d3)methyl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-9-fluoro-2-((methoxy-d3)methyl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-((methoxy-d3)methyl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the (3S,5R)-diastereomer of Intermediate 11 as starting material.

### Example 98. 2-Amino-4-(7-chloro-2-((difluoromethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

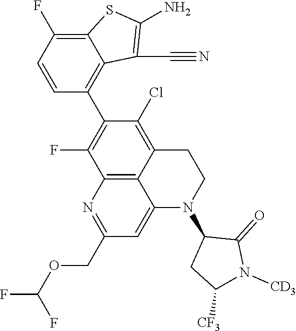 [SMILES: [2H]C([2H])([2H])N1C(=O)[C@H](N2CCc3c(Cl)c(-c4ccc(F)c5sc(N)c(C#N)c45)c(F)c4nc(COC(F)F)cc2c34)C[C@H]1C(F)(F)F]

### Step 1. (3R,5S)-3-(8-Bromo-7-chloro-2-((difluoromethoxy)methyl)-9-fluoro-5,6-dihydro-4H-benzo[de][1,6]naphthyridin-4-yl)-1-(methyl-d₃)-5-(trifluoromethyl)pyrrolidin-2-one

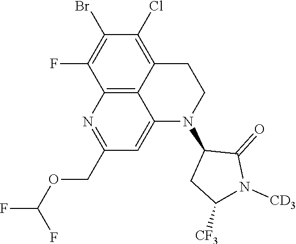 [SMILES: [2H]C([2H])([2H])N1C(=O)[C@H](N2CCc3c(Cl)c(Br)c(F)c4nc(COC(F)F)cc2c34)C[C@H]1C(F)(F)F]

In a 1 dram vial was added (3R,5S)-3-(8-bromo-7-chloro-9-fluoro-2-(hydroxymethyl)-5,6-dihydro-4H-benzo[de][1,6]naphthyridin-4-yl)-1-(methyl-d₃)-5-(trifluoromethyl)pyrrolidin-2-one (Intermediate 15, 21.7 mg, 0.043 mmol) and KOAc (25.6 mg, 0.261 mmol), the vial was capped, then evacuated and back filled with N₂. Then DCM (109 μL), water (109 μL), and (bromodifluoromethyl)trimethylsilane (27.1 μL, 0.174 mmol) were added. The resulting mixture was stirred at r.t. overnight. The reaction was quenched with sat. aq. NaHCO₃, diluted with EtOAc, phase separated, and the organic phase was washed with brine, dried over Na₂SO₄, filtered, and concentrated. The crude product was purified by FCC (10-100% EtOAc in hexanes) to provide the title compound (4.9 mg, 21%). LCMS calc. for C₁₉H₁₃D₃BrClF₆N₃O₂ (M+H)⁺ m/z=549.0, 551.0; found 549.0, 551.0.

### Step 2. 2-Amino-4-(7-chloro-2-((difluoromethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (9.5 mg, 0.065 mmol), tert-butyl (3-cyano-4-(5,5-dimethyl-1,3,2-dioxaborinan-2-yl)-7-fluorobenzo[b]thiophen-2-yl)carbamate (18.2 mg, 0.045 mmol), (3R,5S)-3-(8-bromo-7-chloro-2-(((1,1-dioxidothietan-3-yl)oxy)methyl)-9-fluoro-5,6-dihydro-4H-benzo[de][1,6]naphthyridin-4-yl)-1-(methyl-d₃)-5-(trifluoromethyl)pyrrolidin-2-one (4.9 mg, 0.009 mmol), (2R,3R)-BaryPhos (1.5 mg, 2.9 μmol), and Pd(dba)₂ (1.5 mg, 2.7 μmol) was added DCE (89 μL) under N₂. The mixture was heated to 50° C. for 4 h. The mixture was cooled to r.t., diluted with EtOAc and filtered. The filtrate was concentrated, and the residue was purified by FCC (10-100% EtOAc in hexanes). The intermediate was dissolved in TFA (1 mL) and the mixture was stirred at r.t. for 10 min. before being concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₂₈H₁₇D₃ClF₇N₅O₂S (M+H)⁺ m/z=661.1; found 661.1.

The compounds are understood to comprise atropisomers, i.e., (Rₐ)-2-amino-4-(7-chloro-2-((difluoromethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((difluoromethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((difluoromethoxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((difluoromethoxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using (3S,5R)-diastereomer of Intermediate 11 as starting material.

### Example 99. 2-Amino-4-(7-chloro-2-((2-cyanoethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

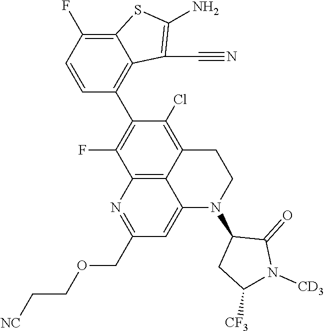 [SMILES: [2H]C([2H])([2H])N1C(=O)[C@H](N2CCc3c(Cl)c(-c4ccc(F)c5sc(N)c(C#N)c45)c(F)c4nc(COCCC#N)cc2c34)C[C@H]1C(F)(F)F]

### Step 1. 3-((8-Bromo-7-chloro-9-fluoro-4-((3R,5S)-1-(methyl-d3)-2-oxo-5-(trifluoromethyl)pyrrolidin-3-yl)-5,6-dihydro-4H-benzo[de][1,6]naphthyridin-2-yl)methoxy)propanenitrile

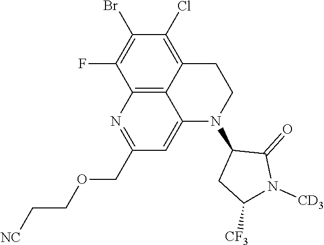 [SMILES: [2H]C([2H])([2H])N1C(=O)[C@H](N2CCc3c(Cl)c(Br)c(F)c4nc(COCCC#N)cc2c34)C[C@H]1C(F)(F)F]

To a solution of (3R,5S)-3-(8-bromo-7-chloro-9-fluoro-2-(hydroxymethyl)-5,6-dihydro-4H-benzo[de][1,6]naphthyridin-4-yl)-1-(methyl-d₃)-5-(trifluoromethyl)pyrrolidin-2-one (Intermediate 15, 50.2 mg, 0.100 mmol) in THF (502 μL) were added DBU (18 μL, 0.12 mmol) and acrylonitrile (67 μL, 1.0 mmol) at r.t. under N₂. The resulting mixture was warmed to 70° C. for 6 h before cool to r.t., diluted with EtOAc, wash with brine, dried over Na₂SO₄, filtered, and concentrated. The crude product was purified by FCC (10-80% EtOAc in hexanes) to provide the title compound (26.1 mg, 47%). LCMS calc. for C₂₁H₁₆D₃BrClF₄N₄O₂ (M+H)⁺ m/z=552.0, 554.0; found 552.0, 554.0.

### Step 2. 2-Amino-4-(7-chloro-2-((2-cyanoethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (25.1 mg, 0.118 mmol), tert-butyl (3-cyano-4-(5,5-dimethyl-1,3,2-dioxaborinan-2-yl)-7-fluorobenzo[b]thiophen-2-yl)carbamate (47.7 mg, 0.118 mmol), (3R,5S)-3-(8-bromo-7-chloro-2-(((1,1-dioxidothietan-3-yl)oxy)methyl)-9-fluoro-5,6-dihydro-4H-benzo[de][1,6]naphthyridin-4-yl)-1-(methyl-d₃)-5-(trifluoromethyl)pyrrolidin-2-one (26.1 mg, 0.047 mmol), (2R,3R)-BaryPhos (6.2 mg, 0.012 μmol), and Pd(dba)₂ (6.0 mg, 10.4 μmol) was added DCE (472 μL) under N₂. The mixture was warmed to 50° C. for 5 h. The mixture was cooled to r.t., diluted with EtOAc and filtered. The filtrate was concentrated, and the residue was purified by FCC (10-100% EtOAc in hexanes). The intermediate was dissolved in TFA (1 mL) and the mixture was stirred at r.t. for 10 min. before being concentrated, then diluted with MeCN and purified by prep. HPLC (XBRIDGE® C₁₈ column, eluting with a gradient of MeCN/water containing 0.1% NH₄OH, at flow rate of 60 mL/min.) to afford the title compound. LCMS calc. for C₃₀H₂₀D₃ClF₅N₆O₂S (M+H)⁺ m/z=664.1; found 664.1.

The compounds are understood to comprise atropisomers, i.e., (Rₐ)-2-amino-4-(7-chloro-2-((2-cyanoethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((2-cyanoethoxy)methyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((2-cyanoethoxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((2-cyanoethoxy)methyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using (3S,5R)-diastereomer of Intermediate 11 as starting material.

### Example 100a and 100b. 2-Amino-4-(7-chloro-9-fluoro-2-(hydroxymethyl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

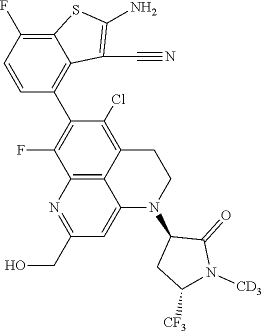 [SMILES: [2H]C([2H])([2H])N1C(=O)[C@H](N2CCc3c(Cl)c(-c4ccc(F)c5sc(N)c(C#N)c45)c(F)c4nc(CO)cc2c34)C[C@H]1C(F)(F)F]

In a 1 dram vial, (3R,5S)-3-(8-bromo-7-chloro-9-fluoro-2-(hydroxymethyl)-5,6-dihydro-4H-benzo[de][1,6]naphthyridin-4-yl)-1-(methyl-d3)-5-(trifluoromethyl)pyrrolidin-2-one (Intermediate 15, 20 mg, 0.040 mmol), tert-butyl (3-cyano-4-(5,5-dimethyl-1,3,2-dioxaborinan-2-yl)-7-fluorobenzo[b]thiophen-2-yl)carbamate (32.4 mg, 0.040 mmol), dichloro[bis(diphenylphosphinophenyl)ether]palladium(II) (8.6 mg, 12 μmol), and K₂CO₃ (16.6 mg, 0.120 mmol) were dissolved in 1,4-dioxane (200 μL) under N₂. The reaction mixture was stirred at 100° C. for 1 h. The reaction was diluted with MeCN and filtered. The filtrate was diluted with DCM and the layers were separated. The organic layer was washed with brine and concentrated. To the crude was added TFA (5 mL), and the resulting mixture was stirred at r.t. for 30 min., then concentrated. The residue was dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 100a. Diastereomer 1. Peak 1. LCMS calc. for C₂₇H₁₇D₃ClF₅N₅O₂S (M+H)⁺ m/z=611.1; found 611.1. This is the potent peak.

Example 100b. Diastereomer 2. Peak 2. LCMS calc. for C₂₇H₁₇D₃ClF₅N₅O₂S (M+H)⁺ m/z=611.1; found 611.1.

The compounds are understood to comprise atropisomers, i.e., (Rₐ)-2-amino-4-(7-chloro-9-fluoro-2-(hydroxymethyl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(hydroxymethyl)-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e.: (Rₐ)-2-amino-4-(7-chloro-9-fluoro-2-(hydroxymethyl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-(hydroxymethyl)-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using (3S,5R)-diastereomer of Intermediate 11 as starting material.

### Example 101a. 2-Amino-4-(7-chloro-2-((1r,3R)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

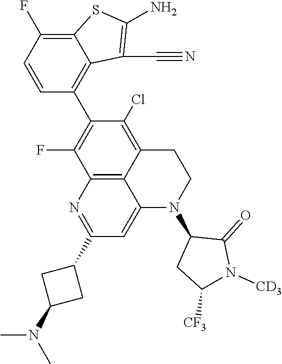 [SMILES: [2H]C([2H])([2H])N1C(=O)[C@H](N2CCc3c(Cl)c(-c4ccc(F)c5sc(N)c(C#N)c45)c(F)c4nc([C@H]5C[C@H](N(C)C)C5)cc2c34)C[C@H]1C(F)(F)F]

### Example 101b. 2-Amino-4-(7-chloro-2-((1s,3S)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

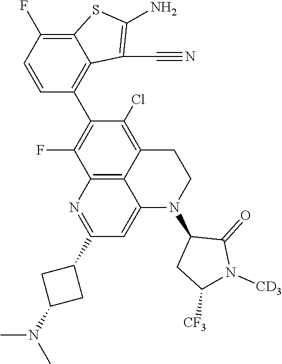 [SMILES: [2H]C([2H])([2H])N1C(=O)[C@H](N2CCc3c(Cl)c(-c4ccc(F)c5sc(N)c(C#N)c45)c(F)c4nc([C@H]5C[C@@H](N(C)C)C5)cc2c34)C[C@H]1C(F)(F)F]

### Step 1. tert-Butyl (3-(8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-2-yl)cyclobutyl)carbamate

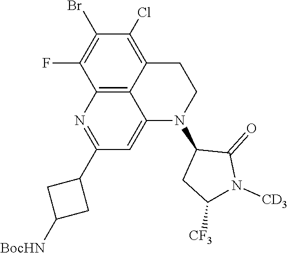 [SMILES: [2H]C([2H])([2H])N1C(=O)[C@H](N2CCc3c(Cl)c(Br)c(F)c4nc(C5CC(NC(=O)OC(C)(C)C)C5)cc2c34)C[C@H]1C(F)(F)F]

In a 40 mL vial, 2,2′-dipyridyl (14.9 mg, 0.095 mmol) and nickel (II) chloride, dimethoxyethane adduct (20.9 mg, 0.095 mmol) were dissolved in DMA (3173 μL). The mixture was stirred at r.t. for 30 min. and then (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (189 mg, 0.317 mmol), tert-butyl ((1r,3r)-3-iodocyclobutyl)carbamate (113 mg, 0.381 mmol), and zinc (31.1 mg, 0.476 mmol) were added sequentially as solids under an N₂ stream. The mixture was re-capped under N₂ and put on an N₂ line. The mixture was stirred at r.t. overnight. The mixture was extracted with EtOAc and the organic layer was washed with aq. LiCl (10% w/v), dried over MgSO₄, filtered, and concentrated. The crude residue was purified by FCC (0-60% EtOAc in hexanes) to give product (123 mg, 61%) as a white solid. LCMS calc. for C₂₆H₂₆D₃BrClF₄N₄O₃ (M+H)⁺ m/z=638.2, 640.2; found 638.2, 640.2.

### Step 2. (3R,5S)-3-(7-Chloro-2-(3-(dimethylamino)cyclobutyl)-9-fluoro-8-methyl-5,6-dihydro-4H-benzo[de][1,6]naphthyridin-4-yl)-1-(methyl-d₃)-5-(trifluoromethyl)pyrrolidin-2-one

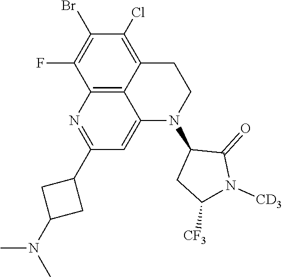 [SMILES: [2H]C([2H])([2H])N1C(=O)[C@H](N2CCc3c(Cl)c(Br)c(F)c4nc(C5CC(N(C)C)C5)cc2c34)C[C@H]1C(F)(F)F]

A solution of tert-butyl (3-(8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-2-yl)cyclobutyl)carbamate (123 mg, 0.193 mmol) in TFA (1 mL) was stirred at r.t. for 15 min. before being concentrated. The residue was mixed with formic acid (807 μL, 21.0 mmol) formaldehyde (805 μL, 10.8 mmol) and heated at 100° C. for 1 h. The mixture was cooled to 0° C. and quenched with aq. 30% NH₄OH and extracted with EtOAc. Combined organic layers were washed with brine, dried over MgSO₄, and the solvent was removed in vacuo. The crude product was used directly in the following reaction. LCMS calc. for C₂₄H₂₅D₃ClF₄N₄O (M+H)⁺ m/z=502.2, 504.2; found 502.2, 504.2.

### Step 3. 2-Amino-4-(7-chloro-2-((1r,3R)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (70.2 mg, 0.331 mmol), tert-butyl (3-cyano-4-(5,5-dimethyl-1,3,2-dioxaborinan-2-yl)-7-fluorobenzo[b]thiophen-2-yl)carbamate (134 mg, 0.331 mmol), (3R,5S)-3-(7-chloro-2-(3-(dimethylamino)cyclobutyl)-9-fluoro-8-methyl-5,6-dihydro-4H-benzo[de][1,6]naphthyridin-4-yl)-1-(methyl-d₃)-5-(trifluoromethyl)pyrrolidin-2-one (93.7 mg, 0.165 mmol), (2R,3R)-BaryPhos (17.4 mg, 0.033 μmol), and Pd₂(dba)₃CHCl₃ (30.8 mg, 30 μmol) was added DCE (1653 μL) under N₂. The mixture was warmed to 60° C. for 1 h. The mixture was cooled to r.t., diluted with EtOAc and filtered. The filtrate was concentrated, the residue was dissolved in 1:1 MeCN/MeOH (8 mL) and purified by prep. HPLC (XBRIDGE® C₁₈ column, eluting with a gradient of MeCN/water containing 0.1% TFA, at flow rate of 60 mL/min.) to provide the desired intermediate. The intermediate was dissolved in TFA, and the mixture was stirred at r.t., then concentrated, the residue was dissolved in MeCN and purified by prep. HPLC (XBRIDGE® C₁₈ column, eluting with a gradient of MeCN/water containing 0.1% NH₄OH, at flow rate of 60 mL/min.) to afford the title compound as two peaks.

Example 101a. Diastereomer 1. Peak 1. LCMS calc. for C₃₂H₂₆D₃ClF₅N₆OS (M+H)⁺ m/z=678.2; found 678.2.

Example 101b. Diastereomer 2. Peak 2. LCMS calc. for C₃₂H₂₆D₃ClF₅N₆OS (M+H)⁺ m/z=678.2; found 678.2.

The compounds are understood to comprise atropisomers, i.e., (Rₐ)-2-amino-4-(7-chloro-2-((1r,3R)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((1r,3R)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((1r,3S)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((1r,3S)-3-(dimethylamino)cyclobutyl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((1r,3R)-3-(dimethylamino)cyclobutyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-2-((1r,3R)-3-(dimethylamino)cyclobutyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-2-((1r,3S)-3-(dimethylamino)cyclobutyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((1r,3S)-3-(dimethylamino)cyclobutyl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the ((3S,5R)-diastereomer of Intermediate 11 as starting material.

### Example 102a and 102b. 2-Amino-4-(7-chloro-2-((3S,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

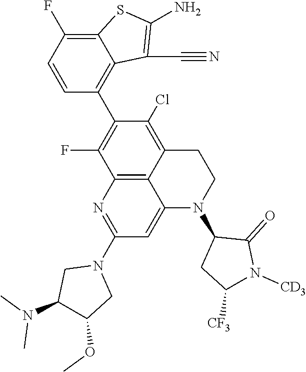 [SMILES: [2H]C([2H])([2H])N1C(=O)[C@H](N2CCc3c(Cl)c(-c4ccc(F)c5sc(N)c(C#N)c45)c(F)c4nc(N5C[C@H](OC)[C@@H](N(C)C)C5)cc2c34)C[C@H]1C(F)(F)F]

### Step 1. tert-Butyl ((3S,4S)-1-(8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-2-yl)-4-methoxypyrrolidin-3-yl)carbamate

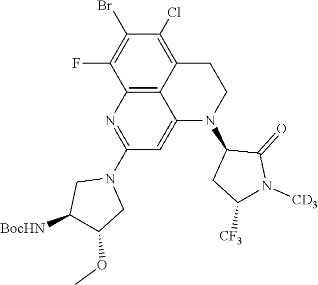 [SMILES: [2H]C([2H])([2H])N1C(=O)[C@H](N2CCc3c(Cl)c(Br)c(F)c4nc(N5C[C@H](NC(=O)OC(C)(C)C)[C@@H](OC)C5)cc2c34)C[C@H]1C(F)(F)F]

In a 1 dram vial, (3R,5S)-3-(8-bromo-7-chloro-9-fluoro-2-iodo-5,6-dihydro-4H-benzo[de][1,6]naphthyridin-4-yl)-1-(methyl-d₃)-5-(trifluoromethyl)pyrrolidin-2-one (Intermediate 13, 150 mg, 0.252 mmol), tert-butyl ((3R,4R)-4-methoxypyrrolidin-3-yl)carbamate (163 mg, 0.756 mmol), L-proline (5.4 mg, 0.047 mmol), CuI (9.0 mg, 0.047 mmol) and K₂CO₃ (104 mg, 0.756 mmol) were dissolved in DMSO (1259 μL) under N₂. The reaction mixture was heated to 80° C. When done, the reaction mixture was diluted with 30% aq. NH₄OH, then extracted with EtOAc. The organic layer was washed with 10% aq. LiCl, dried over MgSO₄ and concentrated. The crude product was used directly in the following reaction. LCMS calc. for C₂₇H₂₉D₃BrClF₄N₅O₄ (M+H)⁺ m/z=683.1, 685.1; found 683.1, 685.1.

### Step 2. (3R,5S)-3-(8-Bromo-7-chloro-2-((3S,4S)-3-(dimethylamino)-4-methoxypyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one

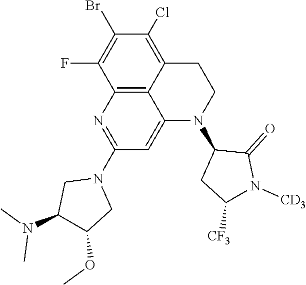 [SMILES: [2H]C([2H])([2H])N1C(=O)[C@H](N2CCc3c(Cl)c(Br)c(F)c4nc(N5C[C@H](OC)[C@@H](N(C)C)C5)cc2c34)C[C@H]1C(F)(F)F]

In a 40 mL vial, tert-butyl ((3R,4R)-1-(8-bromo-7-chloro-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-2-yl)-4-methoxypyrrolidin-3-yl)carbamate (172 mg, 0.251 mmol) was dissolved in TFA (2515 μl) under N₂ with stirring. The reaction mixture was immediately put on a heat block at 50° C. and N₂ was purged through headspace to evaporate the TFA. The residue was placed on high vacuum to remove residual TFA and the residue was treated with formaldehyde (1498 μL, 20.12 mmol) and formic acid (1495 μL, 39.0 mmol) and was heated at 100° C. for 2 h. The reaction mixture was cooled to 0° C. and quenched with 30% aq. NH₄OH and extracted with DCM. The organic layer was phase separated, concentrated, and the residue was purified by prep. SFC (4-14% MeOH in CO₂, 2-PIC column, 140 mL/min. flow rate) to provide the title compound (68.4 mg, 45%). LCMS calc. for C₂₄H₂₅D₃BrClF₄N₅O₂ (M+H)⁺ m/z=611.1, 613.1; found 611.1, 613.1.

### Step 3. 2-Amino-4-(7-chloro-2-((3S,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

To the mixture of K₃PO₄ (47.5 mg, 0.224 mmol), tert-butyl (3-cyano-4-(5,5-dimethyl-1,3,2-dioxaborinan-2-yl)-7-fluorobenzo[b]thiophen-2-yl)carbamate (90 mg, 0.224 mmol), (3R,5S)-3-(8-bromo-7-chloro-2-((3S,4S)-3-(dimethylamino)-4-methoxypyrrolidin-1-yl)-9-fluoro-5,6-dihydro-4H-benzo[de][1,6]naphthyridin-4-yl)-1-(methyl-d₃)-5-(trifluoromethyl)pyrrolidin-2-one (68.4 mg, 0.112 mmol), (2R,3R)-BaryPhos (11.7 mg, 0.022 μmol), and Pd₂(dba)₃CHCl₃ (23.1 mg, 22 μmol) was added DCE (1490 μL) under N₂. The mixture was warmed to 80° C. for 3 h. The mixture was cooled to r.t., diluted with water, extracted with DCM, phase separated, dried over MgSO4, filtered, and concentrated. The residue was dissolved in TFA, and the mixture was stirred at r.t. until full conversion, then concentrated. The residue was dissolved in MeCN and purified by prep. HPLC (XBRIDGE® Cl₈ column, eluting with a gradient of MeCN/water containing 0.1% NH₄OH, at flow rate of 60 mL/min.) to afford the title compound as two peaks.

Example 102a. Diastereomer 1. Peak 1. LCMS calc. for C₃₃H₂₉D₃ClF₅N₇O₂S (M+H)⁺ m/z=723.2; found 723.2.

Example 102b. Diastereomer 1. Peak 2. LCMS calc. for C₃₃H₂₉D₃ClF₅N₇O₂S (M+H)⁺ m/z=723.2; found 723.2.

The compounds are understood to comprise atropisomers, i.e.,
(Rₐ)-2-amino-4-(7-chloro-2-((3S,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3S,4S)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Rₐ)-2-amino-4-(7-chloro-2-((3R,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3R,4R)-3-(dimethylamino)-4-methoxypyrrolidin-1-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e.: (Rₐ)-2-amino-4-(7-chloro-2-((3S,4S)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3S,4S)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Rₐ)-2-amino-4-(7-chloro-2-((3R,4R)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((3R,4R)-3-(dimethylamino)-4-methoxypyrrolidin-1-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the (3S,5R)-enantiomer of Intermediate 11 as starting material.

### Example 103. 2-amino-4-(7-chloro-2-(4-(dimethylamino)-2-azabicyclo[2.1.1]hexan-2-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile

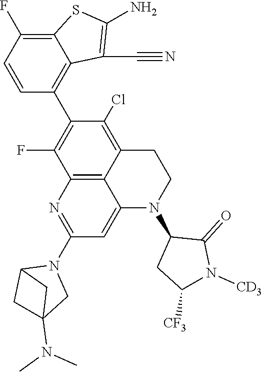 [SMILES: [2H]C([2H])([2H])N1C(=O)[C@H](N2CCc3c(Cl)c(-c4ccc(F)c5sc(N)c(C#N)c45)c(F)c4nc(N5CC6(N(C)C)CC5C6)cc2c34)C[C@H]1C(F)(F)F]

This compound was prepared according to the general procedures outlined for Example 102 using the appropriate starting material as the coupling partner in Step 1. LCMS calc. for C₃₃H₂₇D₃ClF₅N₇O₂S (M+H)⁺ m/z=705.2; found 705.2.

The compounds are understood to comprise atropisomers, i.e., (Rₐ)-2-amino-4-(7-chloro-2-((1R,4R)-4-(dimethylamino)-2-azabicyclo[2.1.1]hexan-2-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((1R,4R)-4-(dimethylamino)-2-azabicyclo[2.1.1]hexan-2-yl)-9-fluoro-4-((3R,5S)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile.

The alternative isomers, i.e., (Rₐ)-2-amino-4-(7-chloro-2-((1S,4S)-4-(dimethylamino)-2-azabicyclo[2.1.1]hexan-2-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-2-((1S,4S)-4-(dimethylamino)-2-azabicyclo[2.1.1]hexan-2-yl)-9-fluoro-4-((3S,5R)-1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile; are prepared by an analogous procedure using the (3S,5R)-enantiomer of Intermediate 11 as starting material.

### Examples 104-105

Compounds in Table 13 were prepared according to the general procedure described for Example 16, using the appropriate amine coupling partner in the reductive amination/cyclization.

TABLE 13

| Ex. | Structure | Name | [M + H] |
|---|---|---|---|
| 104a/b | 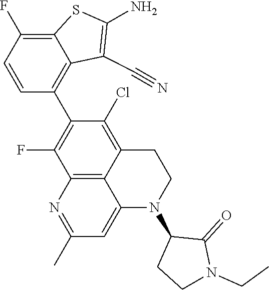 [SMILES: CCN1CC[C@@H](N2CCc3c(Cl)c(-c4ccc(F)c5sc(N)c(C#N)c45)c(F)c4nc(C)cc2c34)C1=O] | 2-amino-4-(7-chloro-4-((R)-1-ethyl- 2-oxopyrrolidin-3-yl)-9-fluoro-2- methyl-5,6-dihydro-4H- benzo[de][1,6]naphthyridin-8-yl)-7- fluorobenzo[b]thiophene-3- carbonitrile | 538.1 |
| 105a/b | 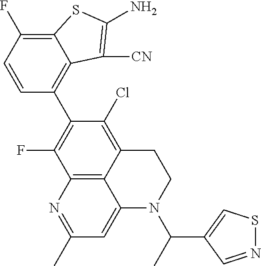 [SMILES: Cc1cc2c3c(c(Cl)c(-c4ccc(F)c5sc(N)c(C#N)c45)c(F)c3n1)CCN2C(C)c1cnsc1] | 2-amino-4-(7-chloro-9-fluoro-4-(1- (isothiazol-4-yl)ethyl)-2-methyl-5,6- dihydro-4H- benzo[de][1,6]naphthyridin-8-yl)-7- fluorobenzo[b]thiophene-3- carbonitrile | 538.1 |

### Example 104

The compounds of Example 104a and 104b are understood to comprise diastereoisomers and/or atropisomers, i.e., (Rₐ)-2-amino-4-(7-chloro-4-((R)-1-ethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((R)-1-ethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((S)-1-ethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((S)-1-ethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Example 105

The compounds of Example 105a and 105b are understood to comprise atropisomers and/or diastereoisomers, i.e., (Rₐ)-2-amino-4-(7-chloro-9-fluoro-4-((R)-1-(isothiazol-4-yl)ethyl)-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-4-((R)-1-(isothiazol-4-yl)ethyl)-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-4-((S)-1-(isothiazol-4-yl)ethyl)-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-4-((S)-1-(isothiazol-4-yl)ethyl)-2-methyl-5,6-dihydro-4H-benzo[de][1,6]naphthyridin-8-yl)-7-fluorobenzo[b]thiophene-3-carbonitrile.

### Examples 106-108

Compounds in Table 14 were prepared according to the general procedure outlined in Example 25 using the appropriate starting material.

TABLE 14

| Example | Structure | Name | [M + H]+ |
|---|---|---|---|
| 106a/b | 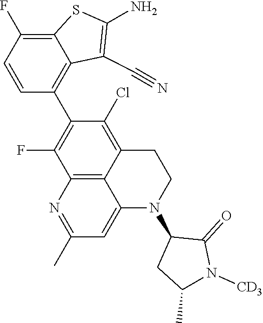 [SMILES: [2H]C([2H])([2H])N1C(=O)[C@H](N2CCc3c(Cl)c(-c4ccc(F)c5sc(N)c(C#N)c45)c(F)c4nc(C)cc2c34)C[C@H]1C] | 2-amino-4-(7-chloro-9-fluoro-2- methyl-4-((3R,5R)-5-methyl-1- (methyl-d₃)-2-oxopyrrolidin-3- yl)-5,6-dihydro-4H- benzo[de][1,6]naphthyridin-8- yl)-7-fluorobenzo[b]thiophene- 3-carbonitrile | 541.1 |
| 107a/b/c | 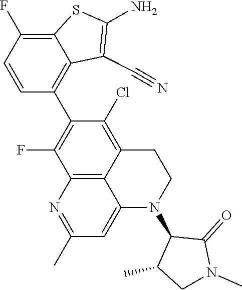 [SMILES: Cc1cc2c3c(c(Cl)c(-c4ccc(F)c5sc(N)c(C#N)c45)c(F)c3n1)CCN2[C@H]1C(=O)N(C)C[C@@H]1C] | 2-amino-4-(7-chloro-4- ((3R,4S)-1,4-dimethyl-2- oxopyrrolidin-3-yl)-9-fluoro-2- methyl-5,6-dihydro-4H- benzo[de][1,6]naphthyridin-8- yl)-7-fluorobenzo[b]thiophene- 3-carbonitrile | 538.1 |
| 108a/b/c | 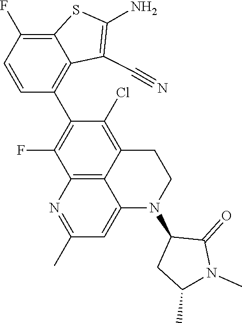 [SMILES: Cc1cc2c3c(c(Cl)c(-c4ccc(F)c5sc(N)c(C#N)c45)c(F)c3n1)CCN2[C@@H]1C[C@@H](C)N(C)C1=O] | 2-amino-4-(7-chloro-4- ((3R,5R)-1,5-dimethyl-2- oxopyrrolidin-3-yl)-9-fluoro-2- methyl-5,6-dihydro-4H- benzo[de][1,6]naphthyridin-8- yl)-7-fluorobenzo[b]thiophene- 3-carbonitrile | 538.1 |

The compounds of Table 14 are understood to comprise mixtures of diastereoisomers and/or atropisomers.

### Example 106a, 106b, and 106c

The compounds Examples 106a, 106b, and 106c are understood to comprise mixtures of diastereoisomers and/or atropisomers, i.e., (Rₐ)-2-amino-4-(7-chloro-9-fluoro-2-methyl-4-((3R,5R)-5-methyl-1-(methyl-d₃)-2-oxo pyrrol id in-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3R,5R)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3S,5S)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3S,5S)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3R,5S)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; (Sₐ)-2-amino-4-(7-chloro-9-fluoro-2-methyl-4-((3R,5S)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; (Rₐ)-2-amino-4-(7-chloro-9-fluoro-2-methyl-4-((3S,5R)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((3S,5R)-5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile.

### Examples 107a, 107b, and 107c

The compounds Examples 107a, 107b, and 107c are understood to comprise mixtures of diastereoisomers and/or atropisomers, i.e., (Rₐ)-2-amino-4-(7-chloro-4-((3R,4S)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3R,4S)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3S,4R)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3S,4R)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3R,4R)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3R,4R)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3S,4S)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,4S)-1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Examples 108a, 108b, and 108c

The compounds Examples 108a, 108b, and 108c are understood to comprise mixtures of diastereoisomers and/or atropisomers, i.e., (Rₐ)-2-amino-4-(7-chloro-4-((3R,5R)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3R,5R)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3S,5S)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3S,5S)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3R,5S)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Sₐ)-2-amino-4-(7-chloro-4-((3R,5S)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; (Rₐ)-2-amino-4-(7-chloro-4-((3S,5R)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and (Sₐ)-2-amino-4-(7-chloro-4-((3S,5R)-1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile.

### Example 109a and 109b. 2-Amino-4-(7-chloro-9-fluoro-2-methyl-4-((R)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile

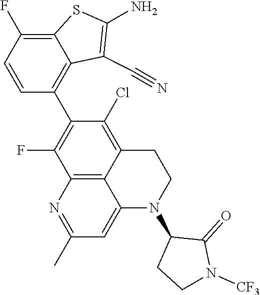 [SMILES: Cc1cc2c3c(c(Cl)c(-c4ccc(F)c5sc(N)c(C#N)c45)c(F)c3n1)CCN2[C@@H]1CCN(C(F)(F)F)C1=O]

### Step 1. tert-Butyl (R)-(2-oxo-1-(trifluoromethyl)pyrrolidin-3-yl)carbamate

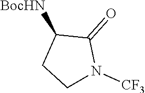 [SMILES: CC(C)(C)OC(=O)N[C@@H]1CCN(C(F)(F)F)C1=O]

To a 40 mL vial was added CsF (379 mg, 2.50 mmol), silver triflate (141 mg, 0.549 mmol), 1-chloromethyl-4-fluoro-1,4-diazoniabicyclo[2.2.2]octane bis(tetrafluoroborate) (708 mg, 2.00 mmol) and tert-butyl (R)-(2-oxopyrrolidin-3-yl)carbamate (100 mg, 0.499 mmol). The vial was evacuated and back filled with N₂ three times. Then DCM (9.4 mL), PhCl (3.1 mL), 2-fluoropyridine (47 μL, 0.55 mmol) and trimethyl(trifluoromethyl)silane (369 μL, 2.50 mmol) were added to the mixture. The resulting mixture was stirred at r.t. overnight before quenching with sat. aq. NaHCO₃. The mixture was then extracted with EtOAc, phase separated, the organic phase was washed with brine, dried over Na₂SO₄, filtered, and concentrated. The crude product was purified by FCC (0-15% MeOH in DCM) to provide the title compound (64.7 mg, 48%). LCMS calc. for C₁₀H₁₆F₃N₂O₃(M+H)⁺ m/z=269.1; found 269.1.

### Step 2. (R)-3-(8-Bromo-7-chloro-9-fluoro-2-methyl-5,6-dihydro-4H-benzo[de][1,6]naphthyridin-4-yl)-1-(trifluoromethyl)pyrrolidin-2-one

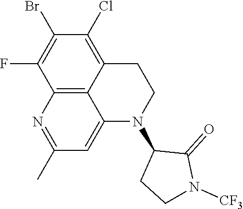 [SMILES: Cc1cc2c3c(c(Cl)c(Br)c(F)c3n1)CCN2[C@@H]1CCN(C(F)(F)F)C1=O]

To a 1 dram vial was added tert-butyl (R)-(2-oxo-1-(trifluoromethyl)pyrrolidin-3-yl)carbamate (64.7 mg, 0.241 mmol) and HCl (4.0 M in 1,4-dioxane, 482 μL, 1.93 mmol) at r.t., the resulting mixture was stirred at r.t. for 95 min. before being concentrated under vacuum to give the product which was used directly for the next step. To a mixture of (R)-3-amino-1-(trifluoromethyl)pyrrolidin-2-one HCl (43.7 mg, 0.214 mmol), THF (366 μL), DCM (366 μL), HFIP (122 μL) was added DIPEA (37 μL, 0.21 mmol). The mixture was stirred at r.t. for 20 min. before Intermediate 2 (30.0 mg, 0.085 mmol), NaBH(OAc)₃ (72.5 mg, 0.342 mmol), and HOAc (66 μL, 0.86 mmol) were added. The mixture was stirred at r.t. overnight. The mixture was quenched with aq. NaHCO₃, extracted with EtOAc, dried over Na₂SO₄, filtered, and concentrated. The crude product was purified by FCC (0-100% EtOAc in hexane) to provide the title compound (42.3 mg, 98%). LCMS calc. for C₁₇H₁₄BrClF₄N₃O (M+H)⁺ m/z=466.0, 468.0; found 466.0, 468.0.

### Step 3. 2-Amino-4-(7-chloro-9-fluoro-2-methyl-4-((R)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile

To a mixture of K₂CO₃ (62.6 mg, 0.453 mmol), [1,1′-bis(diphenylphosphino)ferrocene]dichloropalladium(II) (13.3 mg, 0.018 mmol), tert-butyl (3-cyano-4-(5,5-dimethyl-1,3,2-dioxaborinan-2-yl)-7-fluorobenzo[b]thiophen-2-yl)carbamate (73.3 mg, 0.181 mmol), and (R)-3-(8-bromo-7-chloro-9-fluoro-2-methyl-5,6-dihydro-4H-benzo[de][1,6]naphthyridin-4-yl)-1-(trifluoromethyl)pyrrolidin-2-one (42.3 mg, 0.091 mmol) was added 1,4-dioxane (906 μL) under N₂. The mixture was heated to 90° C. for 51 min. before cooling to r.t., then diluted with EtOAc, filtered and concentrated. The crude product was purified by FCC (0-15% MeOH in DCM). The product was dissolved in DCM/TFA (1:1), then stirred at r.t. for 30 min. The mixture was concentrated. The residue was diluted with MeCN and purified using prep.-LCMS (XBRIDGE® C₁₈ column, eluting with a gradient of MeCN/water containing 0.1% TFA, at flow rate of 60 mL/min.) to afford the title compound as two peaks.

Example 109a. Diastereomer 1. Peak 1. LCMS calc. for C₂₆H₁₈ClF₅N₅OS (M+H)⁺ m/z=578.1; found 578.1.

Example 109b. Diastereomer 2. Peak 2. LCMS calc. for C₂₆H₁₈ClF₅N₅OS (M+H)⁺ m/z=578.1; found 578.1. This is the potent peak.

The compounds are understood to comprise atropisomers and/or diastereoisomers, i.e.,
(Rₐ)-2-amino-4-(7-chloro-9-fluoro-2-methyl-4-((R)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((S)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile; and (Sₐ)-2-amino-4-(7-chloro-9-fluoro-2-methyl-4-((R)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile; and (Rₐ)-2-amino-4-(7-chloro-9-fluoro-2-methyl-4-((S)-2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile.

### Example A. GDP-GTP Exchange Assay

The inhibitor potency of the exemplified compounds was determined in a fluorescence based guanine nucleotide exchange assay, which measures the exchange of bodipy-GDP (fluorescently labeled GDP) for GTP to generate the active state of KRAS in the presence of SOS1 (guanine nucleotide exchange factor). Inhibitors were serially diluted in DMSO and a volume of 0.1 μL was transferred to the wells of a black low volume 384-well plate. 5 μL/well volume of bodipy-loaded KRAS G12V diluted to 2.5 nM in assay buffer (25 mM Hepes pH 7.5, 50 mM NaCl, 10 mM MgCl₂ and 0.01% Brij-35) was added to the plate and pre-incubated with inhibitor for 1 h at r.t. Appropriate controls (enzyme with no inhibitor or with a G12V inhibitor) were included on the plate. The exchange was initiated by the addition of a 5 μL/well volume containing 1 mM GTP and 300 nM SOS1 in assay buffer. The 10 μL/well reaction concentration of the bodipy-loaded KRAS G12V, GTP, and SOS1 were 2.5 nM, 500 μM, and 150 nM, respectively. The reaction plates were incubated at r.t. for 3 h, a time estimated for complete GDP-GTP exchange in the absence of inhibitor. For the KRAS WT, similar guanine nucleotide exchange assays were used with 2.5 nM as final concentration for the bodipy loaded KRAS proteins and 2 h incubation after adding GTP-SOS1 mixture. Internal compounds with confirmed binding were used as positive controls in the assay plates. Fluorescence intensities were measured on a PheraStar plate reader instrument (BMG Labtech) with excitation at 485 nm and emission at 520 nm.

Either GraphPad prism or Genedata Screener SmartFit was used to analyze the data. The IC₅₀ values were derived by fitting the data to a four parameter logistic equation producing a sigmoidal dose-response curve with a variable Hill coefficient.

The KRAS Wild Type (WT) and KRAS_G12V exchange assay IC₅₀ data are provided in Table A below. The symbol “†” indicates IC₅₀≤100 nM, “††” indicates IC₅₀>100 nM but ≤1 μM; and “†††” indicates IC₅₀ is >1 μM but ≤5 μM, “††††” indicates IC₅₀ is >5 μM but ≤10 μM. “NA” indicates IC₅₀ not available.

TABLE A

| Ex. No. | WT exchange | G12V exchange |
|---|---|---|
| 1a | † | † |
| 1b | ††† | †† |
| 2a | † | † |
| 2b | ††† | †† |
| 3a | †† | † |
| 3b | †††† | †† |
| 4a | ††† | †† |
| 4b | †††† | †††† |
| 5a | † | † |
| 5b | †† | †† |
| 6a | † | † |
| 6b | † | † |
| 7a | † | † |
| 7b | †† | † |
| 8a | † | † |
| 8b | †† | †† |
| 9a | † | † |
| 9b | †††† | †† |
| 10a | † | † |
| 10b | ††† | †† |
| 11a | † | † |
| 11b | † | † |
| 11c | ††† | †† |
| 12a | † | † |
| 12b | ††† | †† |
| 13a | † | † |
| 13b | ††† | †† |
| 14a | †† | † |
| 14b | †† | † |
| 15a | † | † |
| 15b | ††† | †† |
| 16a | † | † |
| 16b | ††† | †† |
| 17a | †† | †† |
| 17b | †††† | †††† |
| 18a | †† | †† |
| 18b | †† | ††† |
| 19a | † | † |
| 19b | ††† | †† |
| 20a | † | † |
| 20b | ††† | †† |
| 20c | †† | † |
| 20d | †††† | †† |
| 21a | † | † |
| 21b | ††† | †† |
| 22a | †† | † |
| 22b | ††† | †† |
| 23a | † | † |
| 23b | ††† | †† |
| 24a | ††† | †† |
| 24b | †††† | †††† |
| 25a | †† | † |
| 25b | †† | † |
| 25c | †††† | †† |
| 25d | †††† | ††† |
| 26a | †† | † |
| 26b | †† | †† |
| 27a | †† | † |
| 27b | ††† | †† |
| 27c | †††† | ††† |
| 27d | †††† | †††† |
| 28 | † | † |
| 29a | † | † |
| 29b | ††† | †† |
| 30a | † | † |
| 30b | † | † |
| 31a | ††† | † |
| 31b | †††† | ††† |
| 32a | † | † |
| 32b | †††† | ††† |
| 33a | † | † |
| 33b | † | † |
| 33c | †† | † |
| 33d | ††† | †† |
| 34a | †† | † |
| 34b | †††† | †††† |
| 35a | † | † |
| 35b | †† | †† |
| 36a | † | † |
| 36b | ††† | †† |
| 37 | †† | † |
| 38a | † | † |
| 38b | ††† | ††† |
| 39a | † | † |
| 39b | ††† | †† |
| 40 | †† | † |
| 41a | † | † |
| 41b | †††† | ††† |
| 42a | † | † |
| 42b | †††† | †† |
| 43a | †† | † |
| 43b | ††† | †† |
| 44a | †† | † |
| 44b | ††† | †† |
| 44c | ††† | †† |
| 44d | †† | †† |
| 45a | †††† | †† |
| 45b | †††† | ††† |
| 46a | †† | † |
| 46b | ††† | †† |
| 47a | † | † |
| 47b | ††† | †† |
| 48a | † | † |
| 48b | ††† | †† |
| 49a | † | † |
| 49b | ††† | †† |
| 50a | † | † |
| 50b | †††† | ††† |
| 51 | † | † |
| 52a | † | † |
| 52b | ††† | †† |
| 53a | † | † |
| 53b | ††† | †† |
| 54a | † | † |
| 54b | ††† | ††† |
| 55a | † | † |
| 55b | †† | †† |
| 56a | † | † |
| 56b | †††† | ††† |
| 57a | †† | † |
| 57b | †† | †† |
| 58a | † | † |
| 58b | †† | † |
| 59a | †† | † |
| 59b | ††† | †† |
| 60a | † | † |
| 60b | †††† | ††† |
| 61a | † | † |
| 61b | ††† | †† |
| 62a | † | † |
| 62b | †††† | †††† |
| 63 | †† | † |
| 64a | † | † |
| 64b | ††† | ††† |
| 65a | † | † |
| 65b | ††† | †† |
| 66a | † | † |
| 66b | †††† | †† |
| 67a | †† | † |
| 67b | ††† | †† |
| 68a | † | † |
| 68b | †††† | ††† |
| 69a | † | † |
| 69b | †††† | †† |
| 70a | † | † |
| 70b | ††† | †† |
| 71a | † | † |
| 71b | †† | † |
| 72a | † | † |
| 72b | ††† | ††† |
| 73 | † | † |
| 74 | † | † |
| 75a | †† | † |
| 75b | † | † |
| 75c | †††† | †††† |
| 75d | †††† | †††† |
| 76 | † | † |
| 77a | † | † |
| 77b | † | † |
| 77c | ††† | †† |
| 77d | †††† | ††† |
| 78a | † | † |
| 78b | †††† | †† |
| 79a | †† | † |
| 79b | †††† | †††† |
| 80a | †† | † |
| 80b | †††† | †††† |
| 81a | ††† | †† |
| 81b | †††† | †††† |
| 82 | † | † |
| 83a | † | † |
| 83b | † | † |
| 83c | ††† | †† |
| 84 | † | † |
| 85a | † | † |
| 85b | †††† | ††† |
| 86a | † | † |
| 86b | † | † |
| 87a | † | † |
| 87b | † | † |
| 88a | †††† | † |
| 88b | †††† | †††† |
| 89 | †† | † |
| 90 | † | † |
| 91a | † | † |
| 91b | †† | † |
| 92 | †† | † |
| 93a | †† | † |
| 93b | † | † |
| 93c | ††† | †† |
| 93d | †††† | †††† |
| 94a | † | † |
| 94b | ††† | †† |
| 95 | † | † |
| 96 | † | † |
| 97 | † | † |
| 98 | †† | † |
| 99 | † | † |
| 100a | † | † |
| 100b | †† | †† |
| 101a | † | † |
| 101b | † | † |
| 102a | † | † |
| 102b | † | † |
| 103 | †† | † |
| 104a | † | † |
| 104b | †† | †† |
| 105a | † | † |
| 105b | †† | †† |
| 106a | † | † |
| 106b | †† | † |
| 106c | ††† | †† |
| 107a | ††† | †† |
| 107b | †† | † |
| 107c | ††† | †††† |
| 108a | †††† | ††† |
| 108b | † | † |
| 108c | ††† | †† |
| 109a | †††† | †††† |
| 109b | †† | †† |

### Example B1: Luminescent Viability Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) NCI-H838 (KRAS WT; ATCC® CRL-5844)), BEN (KRAS WT; DSMZ ACC254), and SW620 (KRAS G12V; DSMZ ACC382) cells are cultured in media supplemented with 10% FBS. Eight hundred cells per well in media supplemented with 2% FBS are seeded into white, flat 384-well tissue culture microtiter plates containing 50 nL dots of test compounds (final concentration is a 1:500 dilution, with a final concentration in 0.2% DMSO). Plates are incubated for 3 days at 37° C., 5% CO₂. At the end of the assay, 25 μL/well of CellTiter-Glo reagent (Promega) is added. Luminescence is read after 10 min. with a PHERAstar (BMG). Data are analyzed in Genedata Screener using SmartFit for IC₅₀ values.

### Example B2: Luminescent Viability Assay

NCI-H838 (KRAS WT; ATCC CRL-5844), BEN (KRAS WT; DSMZ ACC254), and SW620 (KRAS G12V; DSMZ ACC382) cells are cultured in media supplemented with 10% FBS. Eight hundred cells per well in media supplemented with 2% FBS are seeded into white, flat 384-well tissue culture microtiter plates containing 50 nL dots of test compounds (final concentration is a 1:500 dilution, with a final concentration in 0.2% DMSO). Plates are incubated for 3 d at 37° C., 5% CO₂. At the end of the assay, 25 μL/well of CellTiter-Glo reagent (Promega) is added. Luminescence is read after 10 min. with a PHERAstar (BMG). Data are analyzed in Genedata Screener using SmartFit for IC₅₀ values.

### Example C1: Cellular pERK HTRF Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) NCI-H838 (KRAS WT; ATCC® CRL-5844) BEN (KRAS WT; DSMZ ACC254), and SW620 (KRAS G12V; DSMZ ACC382) cells are purchased maintained in media supplemented with 10% FBS. The cells are plated at 5000 cells per well (8 μL) into 384-well low volume, flat-bottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h at 37° C., 5% CO₂. 4 μL of 4× lysis buffer with blocking reagent are added to each well and plates are rotated gently (300 rpm) for 30 min. at r.t. 4 μL per well of anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example C2: Cellular pERK HTRF Assay

NCI-H838 (KRAS WT; ATCC® CRL-5844), BEN (KRAS WT; DSMZ ACC254), and SW620 (KRAS G12V; DSMZ ACC382) cells are maintained in media supplemented with 10% FBS. The cells are plated at 5000 cells per well (8 μL) into 384-well low volume, flat-bottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h at 37° C., 5% CO₂. 4 μL of 4× lysis buffer with blocking reagent Revvity are added to each well and plates are rotated gently 20 (300 rpm) for 30 min. at r.t. 4 μL per well of anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example D1: Whole Blood pERK1/2 HTRF Assay

MIA PaCa-2 cells (KRAS G12C; ATCC® CRL-1420), HPAF-II (KRAS G12D; ATCC® CRL-1997), YAPC (KRAS G12V; DSMZ ACC382) and BEN (KRAS WT; DSMZ ACC254) cells are maintained in media supplemented with 10% FBS (Gibco/Life Technologies). For MIA PaCa-2 assay, cells are seeded into 96 well tissue culture plates (Corning #3596) at 25000 cells per well in 100 μL media and cultured for 2 days at 37° C., 5% CO₂ before the assay. For HPAF-II and YAPC assay, cells are seeded in 96 well tissue culture plates at 50000 cells per well in 100 μL media and cultured for 1 day before the assay. Whole Blood is added to the 1 μL dots of compounds (prepared in DMSO) in 96 well plates and mixed gently by pipetting up and down so that the concentration of the compound in blood is 1× of desired concentration, in 0.5% DMSO. The media is aspirated from the cells and 50 μL per well of whole blood with test compound is added and incubated for 4 h at 37° C., 5% CO₂. After the 4 h incubation, the plates are washed once in PBS using the centrifugal Blue Washer (BlueCat Bio) with PBS. 50 μL/well of 1× lysis buffer (Revvity) with blocking reagent (Revvity) and Benzonase nuclease (1:10000 dilution, Sigma) is then added and incubated at r.t. for 30 min. with shaking (250 rpm). Following lysis, 16 μL of lysate is transferred into 384-well small volume white plate (Greiner) using an Assist Plus (Integra Biosciences, NH). 4 μL of 1:1 mixture of anti Phospho-ERK 1/2 d2 and anti Phospho-ERK 1/2 Cryptate (Revvity) is added to the wells using the Assist Plus and incubated at r.t. overnight in the dark. Plates are read on the Pherastar plate reader at 665 nm and 620 nm wavelengths. Data are analyzed in Genedata Screener using SmartFit for IC₅₀values.

### Example D2: Whole Blood pERK1/2 HTRF Assay

BEN (KRAS WT; DSMZ ACC254), and YAPC (KRAS G12V; DSMZ ACC382) are maintained in media supplemented with 10% FBS. Cells are seeded in 96 well tissue culture plates at 50000 cells per well in 100 μL media and cultured for 1 d before the assay. Whole blood is added to the 1 μL dots of compounds (prepared in DMSO) in 96 well plates and mixed gently by pipetting up and down so that the concentration of the compound in blood is 1× of desired concentration, in 0.6% DMSO. The media is aspirated from the cells and 50 μL per well of whole blood with test compound is added and incubated for 4 h at 37° C., 5% CO₂. After the 4 h incubation, the plates are washed once in PBS using the centrifugal Blue Washer (BlueCat Bio) with PBS. 50 μL/well of 1× lysis buffer (Revvity) with blocking reagent (Revvity) and Benzonase nuclease (1:10000 dilution Sigma) is then added and incubated at r,t, for 30 min. with shaking (250 rpm). Following lysis, 16 μL of lysate is transferred into 384-well small volume white plate (Greiner) using an Assist Plus (Integra Biosciences, NH). 4 μL of 1:1 mixture of anti Phospho-ERK 1/2 d2 and anti Phospho-ERK 1/2 Cryptate (Revvity) is added to the wells using the Assist Plus and incubated at r.t. overnight in the dark. Plates are read on the Pherastar plate reader at 665 nm and 620 nm wavelengths. Data are analyzed in Genedata Screener using SmartFit for IC₅₀values.

### Example E: Ras Activation Elisa

The 96-Well Ras Activation ELISA Kit (Cell Biolabs Inc; #STA441) uses the Raf1 RBD (Rho binding domain) bound to a 96-well plate to selectively pull down the active form of Ras from cell lysates. The captured GTP-Ras is then detected by a pan-Ras antibody and HRP-conjugated secondary antibody.

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are maintained in RPMI 1640 with 10% FBS (Gibco/Life Technologies). The cells are seeded into 96 well tissue culture plates (Corning #3596) at 25000 cells per well in 100 μL media and cultured for 2 days at 37° C., 5% CO₂ so that they are approximately 80% confluent at the start of the assay. The cells are treated with compounds for either 4 h or overnight at 37° C., 5% CO₂. At the time of harvesting, the cells are washed with PBS, drained well and then lysed with 50 μL of the 1× Lysis buffer (provided by the kit) plus added Halt Protease and Phosphatase inhibitors (1:100) for 1 h on ice.

The Raf-1 RBD is diluted 1:500 in Assay Diluent (provided in kit) and 100 μL of the diluted Raf-1 RBD is added to each well of the Raf-1 RBD Capture Plate. The plate is covered with a plate sealing film and incubated at r.t. for 1 h on an orbital shaker. The plate is washed 3 times with 250 μL 1× Wash Buffer per well with thorough aspiration between each wash. 50 μL of Ras lysate sample (10-100 μg) is added per well in duplicate. A “no cell lysate” control is added in a couple of wells for background determination. 50 μL of Assay Diluent is added to all wells immediately to each well and the plate is incubated at r.t. for 1 h on an orbital shaker. The plate is washed 5 times with 250 μL 1× Wash Buffer per well with thorough aspiration between each wash. 100 μL of the diluted Anti-pan-Ras Antibody is added to each well and the plate is incubated at r.t. for 1 h on an orbital shaker. The plate is washed 5 times as previously. 100 μL of the diluted Secondary Antibody, HRP Conjugate is added to each well and the plate is incubated at r.t. for 1 h on an orbital shaker. The plate is washed 5 times as previously and drained well. 100 L of Chemiluminescent Reagent (provided in the kit) is added to each well, including the blank wells. The plate is incubated at r.t. for 5 min. on an orbital shaker before the luminescence of each microwell is read on a plate luminometer. The % inhibition is calculated relative to the DMSO control wells after a background level of the “no lysate control” is subtracted from all the values. IC₅₀ determination is performed by fitting the curve of inhibitor percent inhibition versus the log of the inhibitor concentration using the GraphPad Prism 7 software.

### Example F: Inhibition of RAS-RAF and PI3K-AKT Pathways

The cellular potency of compounds is determined by measuring phosphorylation of KRAS downstream effectors extracellular-signal-regulated kinase (ERK), ribosomal S6 kinase (RSK), AKT (also known as protein kinase B, PKB) and downstream substrate S6 ribosomal protein.

To measure phosphorylated extracellular-signal-regulated kinase (ERK), ribosomal S6 kinase (RSK), AKT and S6 ribosomal protein, cells (details regarding the cell lines and types of data produced are further detailed in Table F) are seeded overnight in Corning 96-well tissue culture treated plates in RPMI medium with 10% FBS at 4×104 cells/well. The following day, cells are incubated in the presence or absence of a concentration range of test compounds for 4 h at 37° C., 5% CO₂. Cells are washed with PBS and lysed with 1× lysis buffer (Cisbio) with protease and phosphatase inhibitors (Thermo Fisher, 78446). 10 or 20 μg of total protein lysates is subjected to SDS-PAGE and immunoblot analysis using following antibodies: phospho-ERK1/2-Thr202/Tyr204 (#9101 L), total-ERK1/2 (#9102L), phosphor-AKT-Ser473 (#4060L), phospho-p90RSK-Ser380 (#11989S) and phospho-S6 ribosomal protein-Ser235/Ser236 (#2211S) are from Cell Signaling Technologies (Danvers, MA).

TABLE F

| | | KRAS | |
|---|---|---|---|
| Cell Line | Histology | alteration | Readout |
| H358 | Lung | G12C | pERK, pAKT, p-S6, p-p90RSK |
| MIA PaCa-2 | Pancreas | G12C | pERK, pAKT, p-S6, p-p90RSK |
| HPAF II | Pancreas | G12D | pERK, pAKT, p-S6, p-p90RSK |
| A427 | Lung | G12D | pERK, pAKT, p-S6, p-p90RSK |
| AGS | Stomach | G12D | pERK, pAKT, p-S6, p-p90RSK |
| PaTu 8988s | Pancreas | G12V | pERK, pAKT, p-S6, p-p90RSK |
| H441 | Lung | G12V | pERK, pAKT, p-S6, p-p90RSK |
| YAPC | Pancreas | G12V | pERK, pAKT, p-S6, p-p90RSK |
| SW480 | Colorectal | G12V | pERK, pAKT, p-S6, p-p90RSK |

### Example G1: In Vivo Efficacy Studies

MIA-PaCa-2 (KRAS G12C), H358 (KRAS G12C), HPAF-II (KRAS G12D), AGS (KRAS G12D), SW480 (KRAS G12V) or YAPC (KRAS G12V) human cancer cells are obtained from the American Type Culture Collection or Leibnitz Institute DSMZ and maintained in RPMI media supplemented with 10% FBS. For efficacy studies experiments, 5×106 cells are inoculated subcutaneously into the right hind flank of 6- to 8-week-old BALB/c nude mice (Charles River Laboratories, Wilmington, MA, USA). When tumor volumes are approximately 150-300 mm³, mice are randomized by tumor volume and compounds are orally administered. Tumor volume is calculated using the formula (L×W²)/2, where L and W refer to the length and width dimensions, respectively. Tumor growth inhibition is calculated using the formula (1−(V_{T}/V_{C}))×100, where V_{T} is the average tumor volume of the treatment group on the last day of treatment, and V_{C} is the average tumor volume of the control group on the last day of treatment. Two-way analysis of variance with Dunnett's multiple comparisons test is used to determine statistical differences between treatment groups (GraphPad Prism). Mice are housed at 10-12 animals per cage, and are provided enrichment and exposed to 12 h light/dark cycles. Mice whose tumor volumes exceeded limits (10% of body weight) for two consecutive measurements are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example G: In Vivo Efficacy Studies

MIA-PaCa-2 (KRAS G12C), H358 (KRAS G12C), HPAF-II (KRAS G12D), AGS (KRAS G12D), SW480 (KRAS G12V), SW620 (KRAS G12V) or YAPC (KRAS G12V) human cancer cells are obtained from the American Type Culture Collection and maintained in RPMI media supplemented with 10% FBS. For efficacy studies experiments, 5×10⁶ cells are inoculated subcutaneously into the right hind flank of 6- to 8-week-old NCr nude mice (Taconic Biosciences, Rensselear, NY, USA). When tumor volumes are approximately 150-300 mm3, mice are randomized by tumor volume and compounds are orally administered. Tumor volume is calculated using the formula (L×W²)/2, where L and W refer to the length and width dimensions, respectively. Tumor growth inhibition is calculated using the formula (1−(V_{T}/V_{C}))×100, where V_{T} is the average tumor volume of the treatment group on the last day of treatment, and V_{C} is the average tumor volume of the control group on the last day of treatment. Two-way analysis of variance with Dunnett's multiple comparisons test is used to determine statistical differences between treatment groups (GraphPad Prism). Mice are housed at 10-12 animals per cage, and are provided enrichment and exposed to 12 h light/dark cycles. Mice whose tumor volumes exceeded limits (10% of body weight) for two consecutive measurements are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example H: Caco2 Assay

Caco-2 cells are grown at 37° C. in an atmosphere of 5% CO₂ in DMEM growth medium supplemented with 10% (v/v) fetal bovine serum, 1% (v/v) nonessential amino acids, penicillin (100 U/mL), and streptomycin (100 μg/mL). Confluent cell monolayers are subcultured every 7 days or 4 days for Caco-2 by treatment with 0.05% trypsin containing 1 μM EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values≥300 Ω·cm² are used for transport experiments. To determine the Pₐₚₚ in the absorptive direction (A-B), solution of test compound (50 μM) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{app} ( cm / s ) = ( F * VD ) / ( SA * MD ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

### Example I: Human Whole Blood Stability

The whole blood stability of the exemplified compounds is determined by LCMS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. A 96-deep well analysis plate is prepared with the addition of 100 μL ultrapure water/well. 50 μL chilled ultrapure water/well is added to 96-deep well sample collection plate and covered with a sealing mat. 1 μL of 0.5 M compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4 h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LCMS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example J: In Vitro Intrinsic Clearance Protocol

For in vitro metabolic stability experiments, test compounds are incubated with human liver microsomes at 37° C. The incubation mixture contains test compounds (1 μM), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LCMS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard is achieved using a Hypersil Gold C₁₈ column (50×2.1 mm, 5 μM, 175 Å) from ThermoFisher Scientific (Waltham, MA). Mobile phase A consists of 0.1% formic acid in water, and mobile phase B consists of 0.1% formic acid in MeCN. The total LCMS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) is used. The in vivo hepatic clearance (CL_{H}) is then calculated by using CLᵢₙₜ and hepatic blood flow, Q (20 mL·min⁻¹·kg⁻¹ in humans) in the well-stirred liver model disregarding all binding from CL_{H}=(Q×CLᵢₙₜ)/(Q+CLᵢₙₜ). The hepatic extraction ratio was calculated as CL_{H} divided by Q.

### Example K: In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LCMS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example L: Time Dependent Inhibition (TDI) of CYP Protocol

This assay is designed to characterize an increase in CYP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8 min. in the presence of the substrate cocktail with the addition of a fresh aliquot of NADPH regenerating system. A calibration curve of metabolite standards can be used to quantitatively measure the enzyme activity using LCMS/MS. In addition, incubations with known time dependent inhibitors, tienilic aicd (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LCMS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The −slope is equal to the rate of enzyme loss, or the K_{obs}.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula I: 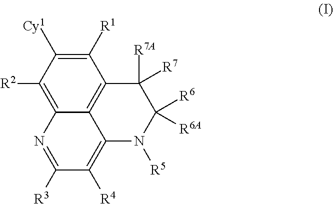
or a pharmaceutically acceptable salt thereof;
wherein
R¹ is selected from C₁₋₃ alkyl, C₁₋₃ haloalkyl, C₃₋₆ cycloalkyl, and halo;
Cy¹ is C₆₋₁₀ aryl or 6-10 membered heteroaryl, each optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{dCy1} is independently selected from H, C₁₋₃ alkyl, and C₁₋₃ haloalkyl;
R² is selected from C₁₋₃ alkyl and halo;
R³ is selected from H, C₁₋₃ alkyl, C₂₋₆ alkenyl, C₂₋₆ alkynyl, C₃₋₁₀ cycloalkyl, 4-10 membered heterocycloalkyl, C₆₋₁₀ aryl, 5-10 membered heteroaryl, halo, D, CN, OR^{a3}, NR^{b3}C(O)R^{a3}, C(O)NR^{b3}R^{c3}, N═S(O)R^{b3}R^{c3}, and NR^{b3}R^{c3}; wherein the C₃₋₁₀cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl, C₂₋₆ alkenyl, and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B}; or
R^{b3} and R^{c3} together with the nitrogen atom to which they are attached form a 4-10 membered heterocycloalkyl, optionally substituted with 1, 2, 3, or 4 substituents independently selected from R^{3A};
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, CN, OR^{d3}, C₃₋₁₀ cycloalkyl, 4-10 membered heterocycloalkyl, S(O)(═NH)C₁₋₆ alkyl, amino, C₁₋₃ alkylamino and di(C₁₋₃ alkyl)amino, wherein the 4-10 membered heterocycloalkyl forming R^{3B} is optionally substituted by 1, 2, 3 or 4 substituents independently selected from R^{3C};
each R^{3C} is independently selected from C₁₋₆ alkyl, C₂₋₆ alkenyl, C₂₋₆ alkynyl, halo and C₁₋₆ haloalkyl, wherein C₁₋₆ alkyl, C₂₋₆ alkenyl, and C₂₋₆ alkynyl are each optionally substituted by 1, 2, or 3 halo;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy⁵ is optionally substituted by Cy^{5A} or (C₃₋₆ alkylene)Cy^{5A} and also optionally substituted by 1, 2, 3, or 4 substituents each independently selected from R^{5C};
each Cy^{5A} is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy^{5A} is optionally substituted by 1, 2, 3, or 4 substituents each independently selected from R^{5C};
R^{5A} is selected from H, D, C₁₋₃ alkyl, C₁₋₃ haloalkyl, and C₃₋₆ cycloalkyl;
R^{5B} is selected from H, D, C₁₋₃ alkyl, and C₁₋₃ haloalkyl;
each R^{5C} is independently selected from C₁₋₆ alkyl and R^{5D}, wherein each C₁₋₆ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, OR^{a5}, SR^{a5}, S(O)R^{a5}, S(O)₂R^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a5}, R^{b5}, R^{c5}, and R^{d5} is independently selected from H and C₁₋₆ alkyl, wherein each C₁₋₆ alkyl forming each R^{a5}, R^{b5}, R^{c5}, and R^{d5} is optionally substituted by 1, 2, 3, or 4 substituents each independently selected from D and halo;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{e5} is independently selected from H, C₁₋₆ alkyl, OH and C₁₋₆ alkoxy, wherein each C₁₋₆ alkyl and C₁₋₆ alkoxy forming R^{e5} is optionally substituted by 1, 2, 3, or 4 substituents each independently selected from D and halo;
R⁶ is selected from H, D, C₁₋₃ alkyl, and C₁₋₃ haloalkyl;
R^{6A} is selected from H, D, C₁₋₃ alkyl, and C₁₋₃ haloalkyl;
R⁷ is selected from H, D, C₁₋₃ alkyl, C₁₋₃ haloalkyl, and halo; and
R^{7A} is selected from H, D, C₁₋₃ alkyl, C₁₋₃ haloalkyl, and halo; or
R⁷ and R^{7A} together with the carbon atom to which they are attached form a spiro fused C₃₋₇ cycloalkane ring;
wherein the ring-forming atoms of each heteroaryl and heterocycloalkyl consist of at least one ring-forming carbon atom and 1, 2, 3, or 4 heteroatoms independently selected from N, O, and S; wherein a ring-forming carbon atom of each heteroaryl and heterocycloalkyl is optionally substituted by oxo to form a carbonyl group.

2. The compound or pharmaceutically acceptable salt thereof of claim 1, wherein
R¹ is selected from C₁₋₃ alkyl and halo;
Cy¹ is C₆₋₁₀ aryl or 6-10 membered heteroaryl, each optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{d}Cy¹ is independently selected from H, C₁₋₃ alkyl, and C₁₋₃ haloalkyl;
R² is selected from C₁₋₃ alkyl and halo;
R³ is selected from H, C₁₋₃ alkyl, C₂₋₆ alkynyl, C₃₋₁₀ cycloalkyl, 4-10 membered heterocycloalkyl, C₆₋₁₀ aryl, 5-10 membered heteroaryl, D, CN, OR^{a3}, NR^{b3}C(O)R^{a3}, N═S(O)R^{b3}R^{c3}, and NR^{b3}R^{c3}; wherein the C₃₋₁₀ cycloalkyl, 4-10 membered heterocycloalkyl, C₆₋₁₀ aryl, and 5-10 membered heteroaryl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3 or 4 substituents each independently selected from R^{3B};
each R^{a3}, R^{b3}, and R^{c3} is independently selected from H, C₁₋₆ alkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{b3}, and R^{c3} are each optionally substituted with 1, 2, 3, or 4 substituents independently selected from R^{3A} and wherein the C₁₋₆ alkyl forming R^{a3}, R^{b3} and R^{c3} is optionally substituted with 1, 2, 3, or 4 substituents independently selected from R^{3B};
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B}, wherein the C₁₋₆ alkyl forming R^{3A} is optionally substituted by 1, 2, 3, or 4 substituents each independently selected from R^{3B};
each R^{3B} is independently selected from halo, D, CN, OR^{d3}, C₃₋₁₀cycloalkyl, S(O)(═NH)C₁₋₆ alkyl, 4-10 membered heterocycloalkyl, amino, C₁₋₃ alkylamino and di(C₁₋₃ alkyl)amino, wherein the 4-10 membered heterocycloalkyl forming R^{3B} is optionally substituted by 1, 2, 3 or 4 substituents independently selected from R^{3C};
each R^{3C} is independently selected from C₁₋₆ alkyl, C₂₋₆ alkenyl halo, and C₁₋₆ haloalkyl, wherein C₂₋₆ alkenyl is optionally substituted by 1, 2, or 3 halo;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the C₃₋₁₀ cycloalkyl, 4-10 membered heterocycloalkyl, C₆₋₁₀ aryl, 5-10 membered heteroaryl forming Cy⁵ is optionally substituted by 1, 2, 3, or 4 substituents each independently selected from R^{5C};
R^{5A} is selected from H, D, and C₁₋₃ alkyl;
R^{5B} is selected from H, D, and C₁₋₃ alkyl;
each R^{5C} is independently selected from C₁₋₃ alkyl and R^{5D}, wherein each C₁₋₃ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, C₁₋₃ alkoxy, amino, C₁₋₃ alkylamino, and di(C₁₋₃ alkyl)amino;
R⁶ is selected from H, D, C₁₋₃ alkyl and C₁₋₃ haloalkyl;
R^{6A} is selected from H, D, C₁₋₃ alkyl and C₁₋₃ haloalkyl;
R⁷ is selected from H, D, C₁₋₃ alkyl and C₁₋₃ haloalkyl; and
R^{7A} is selected from H, D, C₁₋₃ alkyl, and C₁₋₃ haloalkyl; or
R⁷ and R^{7A} together with the carbon atom to which they are attached form a spiro fused C₃₋₇ cycloalkane ring;
wherein the ring-forming atoms of each heteroaryl and heterocycloalkyl consist of at least one ring-forming carbon atom and 1, 2, 3, or 4 heteroatoms independently selected from N, O, and S; wherein a ring-forming carbon atom of each heteroaryl and heterocycloalkyl is optionally substituted by oxo to form a carbonyl group.

3. The compound or pharmaceutically acceptable salt thereof of claim 1, wherein
R¹ is selected from C₁₋₃ alkyl and halo;
Cy¹ is C₆₋₁₀ aryl or 6-10 membered heteroaryl, each optionally substituted with 1, 2, or 3 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, and NR^{cCy1}R^{dCy1};
each R^{aCy1}, R^{cCy1}, and R^{d}Cy¹ is independently selected from H, C₁₋₃ alkyl, and C₁₋₃ haloalkyl;
R² is selected from C₁₋₃ alkyl and halo;
R³ is selected from H, C₁₋₆ alkyl, C₂₋₆ alkynyl, C₃₋₆ cycloalkyl, 4-6 membered heterocycloalkyl, C₆₋₁₀ aryl, 5-10 membered heteroaryl, halo, D, CN, NR^{b3}C(O)R^{a3}, N═S(O)R^{b3}R^{c3} and C(O)NR^{b3}R^{c3}; wherein the 4-10 membered heterocycloalkyl forming R³ is optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3} and R^{c3} is independently selected from H and C₁₋₆ alkyl;
each R^{d3} is independently selected from H, C₁₋₆ alkyl, C₃₋₁₀ cycloalkyl, and 4-10 membered heterocycloalkyl; wherein the C₁₋₆ alkyl forming R^{d3} is optionally substituted with 1, 2, 3, or 4 substituents independently selected from D, halo, CN, S(O₂)C₁₋₆ alkyl;
each R^{3A} is independently selected from C₁₋₆ alkyl and R^{3B};
each R^{3B} is independently di(C₁₋₃ alkyl)amino, S(O)(═NH)C₁₋₆ alkyl, OR^{d3}, or C₃₋₁₀ cycloalkyl;
R⁴ is selected from H, D, C₁₋₃ alkyl, halo, and CN;
R⁵ is selected from Cy⁵ and CR^{5A}R^{5B}Cy⁵;
each Cy⁵ is independently selected from C₃₋₁₀ cycloalkyl, 4-10 membered heterocycloalkyl, C₆₋₁₀ aryl, 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C};
each Cy^{5A} is independently selected from C₃₋₁₀ cycloalkyl and C₆₋₁₀ aryl;
R^{5A} is selected from H, D, C₁₋₃ alkyl, and C₃₋₆ cycloalkyl;
R^{5B} is selected from H, D, and C₁₋₃ alkyl;
each R^{5C} is independently selected from C₁₋₃ alkyl and R^{5D}, wherein each C₁₋₃ alkyl forming R^{5C} is optionally substituted by 1, 2, 3, or 4 substituents each independently selected from R^{5D};
each R^{5D} is independently selected from C₂₋₆ alkenyl, C₂₋₆ alkynyl, halo, D, CN, OH, C₁₋₃ alkoxy, C(O)R^{b5}, and NR^{c5}R^{d5};
each R^{b5}, R^{c5}, and R^{d5} is independently selected from H and C₁₋₆ alkyl;
R⁶ is selected from H, D, and C₁₋₃ alkyl;
R^{6A} is selected from H, D, and C₁₋₃ alkyl;
R⁷ is selected from H, D, halo, and C₁₋₃ alkyl; and
R^{7A} is selected from H, D, halo, and C₁₋₃ alkyl; or
R⁷ and R^{7A} together with the carbon atom to which they are attached form a spiro fused C₃₋₇ cycloalkane ring.

4-5. (canceled)

6. The compound or pharmaceutically acceptable salt thereof of claim 1, wherein the compound of Formula I is a compound of any one of Formulae I-A to I-K: 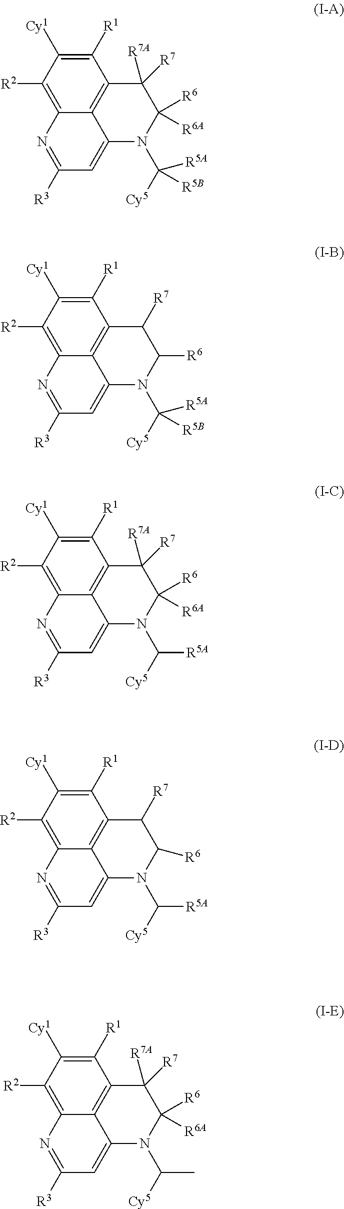 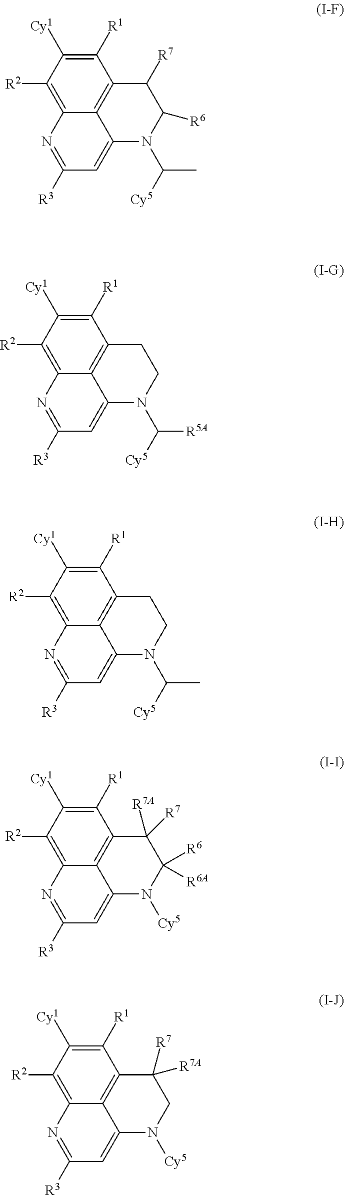 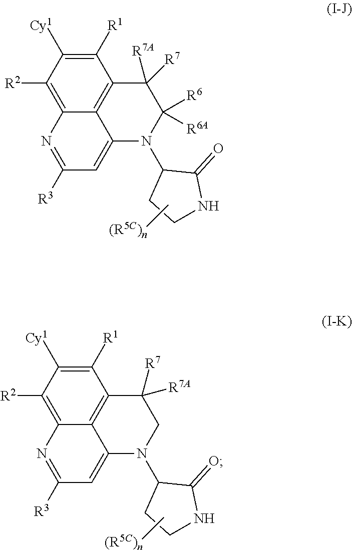
or a pharmaceutically acceptable salt thereof,
wherein n is 0, 1, or 2.

7-9. (canceled)

10. The compound or pharmaceutically acceptable salt thereof of claim 1, wherein R¹ is halo; and R² is halo.

11. (canceled)

12. The compound or pharmaceutically acceptable salt thereof of claim 1, wherein Cy¹ is C₆₋₁₀ aryl or 6-10 membered heteroaryl, each optionally substituted with 1, 2, 3, or 4 substituents independently selected from C₁₋₃ alkyl, C₂₋₆ alkynyl, halo, OH, and NH₂.

13. (canceled)

14. The compound or pharmaceutically acceptable salt thereof of claim 1, wherein each R^{Cy1} is independently selected from C₁₋₃ alkyl, C₂₋₃ alkynyl, C₁₋₃ haloalkyl, halo, CN, and NR^{cCy1}R^{dCy1}.

15-18. (canceled)

19. The compound or pharmaceutically acceptable salt thereof of claim 1, wherein
R³ is selected from H, C₁₋₃ alkyl, C₂₋₆ alkynyl, 4-10 membered heterocycloalkyl, halo, CN, OR^{a3}, NR^{b3}C(O)R^{a3}, and C(O)NR^{b3}R^{c3}; wherein the 4-10 membered heterocycloalkyl forming R³ are each optionally substituted with 1, 2, 3, or 4 substituents independently selected from R^{3A}; and wherein the C₁₋₆ alkyl and C₂₋₆ alkynyl forming R³ is optionally substituted by 1, 2, 3, or 4 substituents each independently selected from R^{3B};
each R^{a3}, R^{b3}, and R^{c3} is independently selected from H, C₁₋₆ alkyl, 4-10 membered heterocycloalkyl, and 5-10 membered heteroaryl; wherein the 4-10 membered heterocycloalkyl and 5-10 membered heteroaryl forming R^{a3}, R^{b3}, and R^{c3} are each optionally substituted with 1, 2, or 3 substituents independently selected from R^{3A} and wherein the C₁₋₆ alkyl forming R^{a3}, R^{b3} and R^{c3} is optionally substituted with 1, 2, or 3 substituents independently selected from R^{3B};
each R^{3A} is independently selected from C₁₋₆ alkyl and R^{3B}, wherein the C₁₋₆ alkyl forming R^{3A} is optionally substituted by 1, 2, or 3, substituents each independently selected from R^{3B};
each R^{3B} is independently selected from halo, OH, C₁₋₃ alkoxy, S(O)(═NH)C₁₋₆ alkyl, 4-10 membered heterocycloalkyl, amino, C₁₋₃ alkylamino and di(C₁₋₃ alkyl)amino, wherein the C₁₋₃ alkoxy and 4-10 membered heterocycloalkyl forming R^{3B} is optionally substituted by 1, 2, 3 or 4 substituents independently selected from R^{3C}; and
each R^{3C} is independently selected from C₁₋₆ alkyl, C₂₋₆ alkenyl, and halo, wherein C₂₋₆ alkenyl is optionally substituted by 1, 2, or 3 halo.

20-29. (canceled)

30. The compound or pharmaceutically acceptable salt thereof of claim 1, wherein R⁴ is H or D.

31-33. (canceled)

34. The compound or pharmaceutically acceptable salt thereof of claim 1, wherein R^{5A} is selected from H, D, C₁₋₃ alkyl, and C₃₋₆ cycloalkyl; and
R^{5B} is selected from H, D, and C₁₋₃ alkyl.

35-43. (canceled)

44. The compound or pharmaceutically acceptable salt thereof of claim 1, wherein Cy⁵ is 4-10 membered heterocycloalkyl and 5-10 membered heteroaryl, wherein the 4-10 membered heterocycloalkyl and 5-10 membered heteroaryl forming Cy⁵ is optionally substituted by Cy^{5A} and also optionally substituted by 1, 2, 3, or 4 substituents each independently selected from R^{5C}.

45-53. (canceled)

54. The compound or pharmaceutically acceptable salt thereof of claim 1, wherein
each R^{5D} is independently halo, D, C(O)R^{b5}, or NR^{c5}R^{d5}; and
each R^{b5}, R^{c5}, and R^{d5} is independently selected from H and C₁₋₆ alkyl.

55-58. (canceled)

59. The compound or pharmaceutically acceptable salt thereof of claim 1, wherein
R⁶ is H;
R^{6a} is H;
R⁷ is H; and
R^{7a} is H.

60-64. (canceled)

65. The compound or pharmaceutically acceptable salt thereof of claim 1, wherein the compound of Formula (I) is selected from 2-amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile;
4-(4-(1-(2-aminopyridin-3-yl)ethyl)-7-chloro-9-fluoro-2-methyl-5,6-dihydro-4H-benzo[de][1,6]naphthyridin-8-yl)-7-fluorobenzo[d]thiazol-2-amine;
8-(4-(1-(2-aminopyridin-3-yl)ethyl)-7-chloro-9-fluoro-2-methyl-5,6-dihydro-4H-benzo[de][1,6]naphthyridin-8-yl)-1-naphthonitrile;
3-(1-(7-chloro-8-(5,6-dimethyl-1H-indazol-4-yl)-9-fluoro-2-methyl-5,6-dihydro-4H-benzo[de][1,6]naphthyridin-4-yl)ethyl)pyridin-2-amine;
2-amino-4-(7-chloro-9-fluoro-2-methyl-4-(1-(pyridin-3-yl)ethyl)-5,6-dihydro-4H-benzo[de][1,6]naphthyridin-8-yl)-7-fluorobenzo[b]thiophene-3-carbonitrile;
2-amino-4-(4-(1-(3-aminopyrazin-2-yl)ethyl)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(4-(1-(3-aminopyridazin-4-yl)ethyl)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(4-(1-(5-aminopyridin-3-yl)ethyl)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(1-methyl-1H-pyrazol-5-yl)ethyl)-5,6-dihydro-4H-benzo[de][1,6]naphthyridin-8-yl)-7-fluorobenzo[b]thiophene-3-carbonitrile;
2-amino-4-(4-(1-(2-aminopyridin-3-yl)ethyl)-7-chloro-9-fluoro-2-methyl-5,6-dihydro-4H-benzo[de][1,6]naphthyridin-8-yl)-7-fluorothieno[3,2-c]pyridine-3-carbonitrile;
2-amino-4-(4-(1-(2-aminopyridin-3-yl)ethyl)-7-chloro-9-fluoro-2,6-dimethyl-5,6-dihydro-4H-benzo[de][1,6]naphthyridin-8-yl)-7-fluorobenzo[b]thiophene-3-carbonitrile;
2-amino-4-(4-(1-(2-aminopyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(4-(1-(2-amino-5-fluoropyridin-3-yl)ethyl-1-d)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(4-((2-aminopyridin-3-yl)(cyclopropyl)methyl-d₃)-7-chloro-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-methyl-2-oxopyrrolidin-3-yl)-5,6-dihydro-4H-benzo[de][1,6]naphthyridin-8-yl)-7-fluorobenzo[b]thiophene-3-carbonitrile;
2-amino-4-(7-chloro-4-(1-cyclopropyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(2-oxo-1-(2,2,2-trifluoroethyl) pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(2-oxo-1-phenylpyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-methyl-2-oxopiperidin-4-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(3-oxooctahydroindolizin-2-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-methyl-2-oxopiperidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(1-methyl-1H-1,2,4-triazol-5-yl)ethyl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile;
4-(4-(1-acetylpyrrolidin-3-yl)-7-chloro-9-fluoro-2-methyl-5,6-dihydro-4H-benzo[de][1,6]naphthyridin-8-yl)-2-amino-7-fluorobenzo[b]thiophene-3-carbonitrile;
2-amino-4-(7-chloro-9-fluoro-2-methyl-4-(1-methyl-2-oxooctahydrocyclopenta[b]pyrrol-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(2-methyl-3-oxo-2-azabicyclo[3.1.0]hexan-4-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(2-methyl-3-oxo-2-azabicyclo[3.2.0]heptan-4-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-3-methylazetidin-1-yl)-9-fluoro-4-(1-methyl-2-oxopyrrolidin-3-yl)-5,6-dihydro-4H-benzo[de][1,6]naphthyridin-8-yl)-7-fluorobenzo[b]thiophene-3-carbonitrile;
8-(2-amino-3-cyano-7-fluorobenzo[b]thiophen-4-yl)-4-(1-(2-aminopyridin-3-yl)ethyl)-7-chloro-9-fluoro-N-methyl-5,6-dihydro-4H-benzo[de][1,6]naphthyridine-2-carboxamide;
2-amino-4-(4-(1-(2-aminopyridin-3-yl)ethyl)-7-chloro-6,6,9-trifluoro-2-methyl-5,6-dihydro-4H-benzo[de][1,6]naphthyridin-8-yl)-7-fluorobenzo[b]thiophene-3-carbonitrile;
3-(8-(2-amino-7-fluorobenzo[d]thiazol-4-yl)-7-chloro-9-fluoro-2-methyl-5,6-dihydro-4H-benzo[de][1,6]naphthyridin-4-yl)-1-methylpyrrolidin-2-one;
8-(2-amino-3-cyano-7-fluorobenzo[b]thiophen-4-yl)-7-chloro-9-fluoro-N-methyl-4-(1-methyl-2-oxopyrrolidin-3-yl)-5,6-dihydro-4H-benzo[de][1,6]naphthyridine-2-carboxamide;
2-amino-4-(7-chloro-9-fluoro-2-(1-hydroxyethyl)-4-(1-methyl-2-oxopyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methoxy-4-(1-methyl-2-oxopyrrolidin-3-yl)-5,6-dihydro-4H-benzo[de][1,6]naphthyridin-8-yl)-7-fluorobenzo [b]thiophene-3-carbonitrile;
8-(2-amino-3-cyano-7-fluorobenzo[b]thiophen-4-yl)-7-chloro-9-fluoro-4-(1-methyl-2-oxopyrrolidin-3-yl)-N-(1-methylazetidin-3-yl)-5,6-dihydro-4H-benzo[de][1,6]naphthyridine-2-carboxamide;
8-(2-amino-3-cyano-7-fluorobenzo[b]thiophen-4-yl)-7-chloro-9-fluoro-N-(2-hydroxy-2-methylpropyl)-4-(1-methyl-2-oxopyrrolidin-3-yl)-5,6-dihydro-4H-benzo[de][1,6]naphthyridine-2-carboxamide;
N-(8-(2-amino-3-cyano-7-fluorobenzo[b]thiophen-4-yl)-7-chloro-9-fluoro-4-(1-methyl-2-oxopyrrolidin-3-yl)-5,6-dihydro-4H-benzo[de][1,6]naphthyridin-2-yl)acetamide;
2-amino-4-(7-chloro-2-((difluoromethoxy)methyl)-9-fluoro-4-(1-methyl-2-oxopyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(4-hydroxy-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile;
2-amino-4-(7-chloro-4-(4-(difluoromethoxy)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-4-(5-(difluoromethyl)-1-(methyl-d₃)-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(4-(methyl-d₃)-5-oxo-4-azaspiro[2.4]heptan-6-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(5-((methoxy-d3)methyl)-1-(methyl-d₃)-2-oxopyrrolidin-3-yl)-2-methyl-5,6-dihydro-4H-benzo[de][1,6]naphthyridin-8-yl)-7-fluorobenzo[b]thiophene-3-carbonitrile;
4-(8-(2-amino-3-cyano-7-fluorobenzo[b]thiophen-4-yl)-7-chloro-9-fluoro-2-methyl-5,6-dihydro-4H-benzo[de][1,6]naphthyridin-4-yl)-N,N-dimethyl-1-(methyl-d₃)-5-oxopyrrolidine-2-carboxamide;
2-amino-4-(7-chloro-9-fluoro-2-methyl-4-(2-methyl-3-oxoisoxazolidin-4-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)azetidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-3-methylazetidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(3-(S-methylsulfonimidoyl) azetidin-1-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-(3-methyl-3-(pyrrolidin-1-yl)azetidin-1-yl)-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-((2-fluorotetrahydro-1H-pyrrolizin-7a(5H)-yl)methoxy)-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-((1-(methyl-d₃)-2-oxo-5-(trifluoromethyl)pyrrolidin-3-yl)-2-(1-(1-methylpyrrolidin-2-yl)ethoxy)-5,6-dihydro-4H-benzo[de][1,6]naphthyridin-8-yl)-7-fluorobenzo[b]thiophene-3-carbonitrile;
2-amino-4-(7-chloro-2-((2-(difluoromethylene)tetrahydro-1H-pyrrolizin-7a(5H)-yl)methoxy)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-((1-methylpyrrolidin-2-yl)methoxy)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(1-(1-methylpyrrolidin-2-yl)ethoxy)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(1H-1,2,4-triazol-1-yl)-5,6-dihydro-4H-benzo[de][1,6]naphthyridin-8-yl)-7-fluorobenzo[b]thiophene-3-carbonitrile;
8-(2-amino-3-cyano-7-fluorobenzo[b]thiophen-4-yl)-7-chloro-9-fluoro-4-(1-(methyl-d₃)-2-oxo-5-(trifluoromethyl)pyrrolidin-3-yl)-N-(tetrahydro-2H-pyran-4-yl)-5,6-dihydro-4H-benzo[de][1,6]naphthyridine-2-carboxamide;
8-(2-amino-3-cyano-7-fluorobenzo[b]thiophen-4-yl)-7-chloro-9-fluoro-4-(1-(methyl-d₃)-2-oxo-5-(trifluoromethyl)pyrrolidin-3-yl)-5,6-dihydro-4H-benzo[de][1,6]naphthyridine-2-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-5-fluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-6,7-difluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-5,7-difluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorothieno[3,2-c]pyridine-3-carbonitrile;
2-amino-7-fluoro-4-(9-fluoro-2-methyl-4-(1-(methyl-d₃)-2-oxo-5-(trifluoromethyl)pyrrolidin-3-yl)-7-(trifluoromethyl)-5,6-dihydro-4H-benzo[de][1,6]naphthyridin-8-yl)benzo[b]thiophene-3-carbonitrile;
2-amino-4-(7-(difluoromethyl)-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-3,9-difluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-7-fluoro-4-(9-fluoro-2,7-dimethyl-4-(1-(methyl-d₃)-2-oxo-5-(trifluoromethyl)pyrrolidin-3-yl)-5,6-dihydro-4H-benzo[de][1,6]naphthyridin-8-yl)benzo[b]thiophene-3-carbonitrile;
2-amino-4-(2-(difluoromethyl)-9-fluoro-7-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(2-(3-(dimethylamino)-3-methylazetidin-1-yl)-9-fluoro-7-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(2-(3-(dimethylamino)azetidin-1-yl)-9-fluoro-7-methyl-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(5-fluoro-1-methyl-2-oxopiperidin-3-yl)-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-(3-hydroxy-3-methylbut-1-yn-1-yl)-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)prop-1-yn-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(5-oxooctahydroindolizin-6-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(1-methyl-1,6-diazaspiro[3.3]heptan-6-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-4-fluoropyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(5-methyl-2,5-diazabicyclo[2.2.1]heptan-2-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(methyl-d₃)-2-oxo-5-phenylpyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)thieno[2,3-b]pyridine-3-carbonitrile;
2-amino-4-(7-chloro-2-((dimethyl(oxo)-λ⁶-sulfaneylidene)amino)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)pyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-3-methylpyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(1-methylhexahydropyrrolo[3,4-b]pyrrol-5 (1H)-yl)-5,6-dihydro-4H-benzo[de][1,6]naphthyridin-8-yl)-7-fluorobenzo[b]thiophene-3-carbonitrile;
2-amino-4-(7-chloro-2-(3-cyclopropyl-3-hydroxyazetidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-3-ethylpyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-cyclopropyl-3-(dimethylamino)pyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridin-8-yl)thieno[2,3-c]pyridine-3-carbonitrile;
8-(2-amino-3-cyano-7-fluorobenzo[b]thiophen-4-yl)-7-chloro-9-fluoro-2-methyl-4-(1-(methyl-d₃)-2-oxo-5-(trifluoromethyl)pyrrolidin-3-yl)-5,6-dihydro-4H-benzo[de][1,6]naphthyridine-3-carbonitrile;
8-(2-amino-3-cyano-7-fluorobenzo[b]thiophen-4-yl)-9-fluoro-2,7-dimethyl-4-(1-(methyl-d₃)-2-oxo-5-(trifluoromethyl)pyrrolidin-3-yl)-5,6-dihydro-4H-benzo[de][1,6]naphthyridine-3-carbonitrile;
2-amino-4-(2-(azetidin-1-ylmethyl)-7-chloro-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-(trifluoromethyl)-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(1-methyl-2-oxo-5-(trifluoromethyl)piperidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-4-methoxypyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(((1,1-dioxidothietan-3-yl)oxy)methyl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(methyl-d₃)-2-oxo-5-(trifluoromethyl)pyrrolidin-3-yl)-2-((2-(methylsulfonyl)ethoxy)methyl)-5,6-dihydro-4H-benzo[de][1,6]naphthyridin-8-yl)-7-fluorobenzo[b]thiophene-3-carbonitrile;
2-amino-4-(7-chloro-9-fluoro-2-((methoxy-d3)methyl)-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-((difluoromethoxy)methyl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-((2-cyanoethoxy)methyl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-9-fluoro-2-(hydroxymethyl)-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)cyclobutyl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(3-(dimethylamino)-4-methoxypyrrolidin-1-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-2-(4-(dimethylamino)-2-azabicyclo[2.1.1]hexan-2-yl)-9-fluoro-4-(1-(methyl-d₃)-2-oxo-5-(trifluoromethyl)pyrrolidin-3-yl)-5,6-dihydro-4H-benzo[de][1,6]naphthyridin-8-yl)-7-fluorobenzo[b]thiophene-3-carbonitrile;
2-amino-4-(7-chloro-4-(1-ethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-4-(1-(isothiazol-4-yl)ethyl)-2-methyl-5,6-dihydro-4H-benzo[de][1,6]naphthyridin-8-yl)-7-fluorobenzo[b]thiophene-3-carbonitrile;
2-amino-4-(7-chloro-9-fluoro-2-methyl-4-(5-methyl-1-(methyl-d₃)-2-oxopyrrolidin-3-yl)-5,6-dihydro-4H-benzo[de][1,6]naphthyridin-8-yl)-7-fluorobenzo[b]thiophene-3-carbonitrile;
2-amino-4-(7-chloro-4-(1,4-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile;
2-amino-4-(7-chloro-4-(1,5-dimethyl-2-oxopyrrolidin-3-yl)-9-fluoro-2-methyl-5,6-dihydro-4H-benzo[de][1,6]naphthyridin-8-yl)-7-fluorobenzo[b]thiophene-3-carbonitrile; and
2-amino-4-(7-chloro-9-fluoro-2-methyl-4-(2-oxo-1-(trifluoromethyl)pyrrolidin-3-yl)-5,6-dihydro-4H-benzo[de][1,6]naphthyridin-8-yl)-7-fluorobenzo[b]thiophene-3-carbonitrile;
and pharmaceutically acceptable salts thereof.

66. A pharmaceutical composition comprising the compound or pharmaceutically acceptable salt thereof according to claim 1, and at least one pharmaceutically acceptable carrier or excipient.

67. A method of inhibiting KRAS activity, the method comprising contacting the compound or pharmaceutically acceptable salt thereof according claim 1, with KRAS.

68. (canceled)

69. The method of claim 67, wherein KRAS is characterized as having a somatic mutation of G12C, G12D, or G12V.

70-71. (canceled)

72. A method of treating a disease or disorder associated with activity of KRAS, the method comprising administering to a patient in need thereof a therapeutically effective amount of the compound or pharmaceutically acceptable salt thereof according to claim 1.

73. A method of treating a disease or disorder associated with activity of a KRAS protein harboring a G12C, G12D, or G12V mutation, the method comprising administering to a patient in need thereof a therapeutically effective amount of the compound or pharmaceutically acceptable salt thereof according to claim 1.

74-75. (canceled)

76. A method for treating a cancer in a patient, the method comprising administering to the patient a therapeutically effective amount of the compound or pharmaceutically acceptable salt thereof according to claim 1.

77. The method of claim 76, wherein the cancer is selected from a carcinoma, a hematological cancer, a sarcoma, and glioblastoma.

78. The method of claim 76, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma; or
the cancer is a carcinoma selected from pancreatic, colorectal, lung, bladder, gastric, esophageal, breast, head and neck, cervical, skin, and thyroid carcinomas.

79. (canceled)

80. The method of claim 76, wherein abnormally proliferating cells of the cancer comprise KRAS having a G12C, G12D, or G12V mutation.

81-82. (canceled)

83. The method of claim 76, wherein the cancer is colorectal cancer, pancreatic cancer, or lung cancer.

84. (canceled)

85. The method of claim 76, wherein the cancer is pancreatic ductal cancer or non-small cell lung cancer (NCSLC).

86-87. (canceled)

88. The method of claim 76, wherein the cancer is metastatic.

89. A method of treating an immunological or inflammatory disorder comprising administering to a patient in need thereof a therapeutically effective amount of the compound or pharmaceutically acceptable salt thereof according to claim 1.

90. The method of claim 89, wherein the immunological or inflammatory disorder is associated with activity of KRAS.

91. The method of claim 89, wherein the immunological or inflammatory disorder is associated with activity of KRAS having a G12C, G12D, or G12V mutation.

92-93. (canceled)

94. The method of claim 89, wherein the immunological or inflammatory disorder is Ras-associated lymphoproliferative disorder or juvenile myelomonocytic leukemia caused by a somatic mutation of KRAS.

95-97. (canceled)
